SEQUENCE LISTING (25 sequences)

SEQ ID NO: 1 - protein, 9 aa, Homo sapiens
IEVMYPPPY

SEQ ID NO: 2 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 288 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 4 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 5 - DNA, 18 bp, Artificial Sequence
  Synthetic Oligonucleotide
cggacctcagtggctttg

SEQ ID NO: 6 - DNA, 20 bp, Artificial Sequence
  Synthetic Oligonucleotide
ttcatccctgtcttctgcaa

SEQ ID NO: 7 - DNA, 24 bp, Artificial Sequence
  Synthetic Oligonucleotide
  misc_feature (1)..(1): 5 prime FAM tag
  misc_feature (9)..(10): ZEN internal quencher
  misc_feature (24)..(24): 3 prime Iowa black tag
cgccagctttgtgtgtgagtatgc

SEQ ID NO: 8 - DNA, 21 bp, Artificial Sequence
  Synthetic Oligonucleotide
cagagacttctgatcccagat

SEQ ID NO: 9 - DNA, 21 bp, Artificial Sequence
  Synthetic Oligonucleotide
tcctgagaagatgcaaccaat

SEQ ID NO: 10 - DNA, 22 bp, Artificial Sequence
  Synthetic Oligonucleotide
  misc_feature (1)..(1): 5 prime FAM tag
  misc_feature (9)..(10): internal ZEN quencher
  misc_feature (22)..(22): 3 prime Iowa Black tag
cccgcccctacatggcttatct

SEQ ID NO: 11 - DNA, 27 bp, Artificial Sequence
  Synthetic Oligonucleotide
gatattgttagtaggtagtctgaatgc

SEQ ID NO: 12 - DNA, 17 bp, Artificial Sequence
  Synthetic Oligonucleotide
ggagctcggtcagacag

SEQ ID NO: 13 - DNA, 28 bp, Artificial Sequence
  Synthetic Oligonucleotide
  misc_feature (1)..(1): 5 prime FAM tag
  misc_feature (9)..(10): internal ZEN quencher
  misc_feature (28)..(28): 3 prime Iowa Black tag
aaagaggcaccagaaggaactttcttga

SEQ ID NO: 14 - DNA, 22 bp, Artificial Sequence
  Synthetic Oligonucleotide
ggtacatgagcagtaggatcag

SEQ ID NO: 15 - DNA, 22 bp, Artificial Sequence
  Synthetic Oligonucleotide
gcagcaacatctttgagagaac

SEQ ID NO: 16 - DNA, 23 bp, Artificial Sequence
  Synthetic Oligonucleotide
  misc_feature (1)..(1): 5 prime FAM tag
  misc_feature (9)..(10): internal ZEN quencher
  misc_feature (23)..(23): 3 prime Iowa Black tag
ccacgatgccacccacaatcaga

SEQ ID NO: 17 - DNA, 19 bp, Artificial Sequence
  Synthetic Oligonucleotide
ttcaggtcctcagctgtca

SEQ ID NO: 18 - DNA, 19 bp, Artificial Sequence
  Synthetic Oligonucleotide
tggctgacacctacgagac

SEQ ID NO: 19 - DNA, 24 bp, Artificial Sequence
  Synthetic Oligonucleotide
  misc_feature (1)..(1): 5 prime FAM tag
  misc_feature (9)..(10): internal ZEN quencher
  misc_feature (24)..(24): 3 prime Iowa Black tag
ccgcctggatgtccttcacaatct

SEQ ID NO: 20 - DNA, 22 bp, Artificial Sequence
  Synthetic Oligonucleotide
gtccattgcctgattctttgtg

SEQ ID NO: 21 - DNA, 20 bp, Artificial Sequence
  Synthetic Oligonucleotide
gtcagtgcaaaccaggaact

SEQ ID NO: 22 - DNA, 24 bp, Artificial Sequence
  Synthetic Oligonucleotide
  misc_feature (1)..(1): 5 prime FAM tag
  misc_feature (9)..(10): internal ZEN quencher
  misc_feature (24)..(24): 3 prime Iowa Black tag
attctgctgagatgtgccggaagt

SEQ ID NO: 23 - DNA, 22 bp, Artificial Sequence
  Synthetic Oligonucleotide
gtagatgagggtaactggcttg

SEQ ID NO: 24 - DNA, 21 bp, Artificial Sequence
  Synthetic Oligonucleotide
ggtgttgggaatctggagatg

SEQ ID NO: 25 - DNA, 25 bp, Artificial Sequence
  Synthetic Oligonucleotide
  misc_feature (1)..(1): 5 prime FAM tag
  misc_feature (9)..(10): internal ZEN quencher
  misc_feature (25)..(25): 3 prime Iowa Black tag
ccttggaaaccacttggcatgttga

(19) United States
(12) Patent Application Publication
(10) Pub. No.: US 2022/0170097 A1
(43) Pub. Date: Jun. 2, 2022

(54) CAR T CELL TRANSCRIPTIONAL ATLAS

(71) Applicants: The Broad Institute, Inc., Cambridge, MA (US); Massachusetts Institute of Technology, Cambridge, MA (US); The General Hospital Corporation, Boston, MA (US)

(72) Inventors: Angela Boroughs, Boston, MA (US); Nemo Marjanovic, Cambridge, MA (US); Aviv Regev, Cambridge, MA (US); Marcela Maus, Boston, MA (US); Tamara Ouspenskaia, Cambridge, MA (US)

(21) Appl. No.: 17/289,844

(22) PCT Filed: Oct. 29, 2019
(86) PCT No.: PCT/US2019/058680

§ 371 Date: Apr. 29, 2021

Related U.S. Application Data
(60) Provisional application No. 62/752,189, filed on Oct. 29, 2018.

(51) Int. Cl. C12Q 1/6881; C12N 5/0783; A61K 35/17; G01N 33/50

(52) U.S. Cl. CPC C12Q 1/6881; C12N 5/0636; C12Q 2600/158; G01N 33/5023; G01N 33/505; A61K 35/17

(57) ABSTRACT

The invention relates to gene expression profiles and signatures of CAR T cells. The invention provides methods and compositions of CAR T cells and populations. The invention provides assays and methods of screening subjects to assess efficacy and safety of CAR T cell treatments and therapies. The invention provides assays and methods of engineering and/or administering CAR T cells to promote efficacy and safety.

### CROSS-REFERENCE TO RELATED APPLICATIONS

This application is a National Stage application of International Application No. PCT/US2019/058680, filed Oct. 29, 2019, which claims the benefit of U.S. Provisional Application No. 62/752,189, filed Oct. 29, 2018. The entire contents of the above-identified applications are hereby fully incorporated herein by reference.

### STATEMENT REGARDING FEDERALLY SPONSORED RESEARCH

This invention was made with government support under Grant No.(s) CA166039 and AI118692 awarded by the National Institutes of Health. The government has certain rights in the invention.

### REFERENCE TO AN ELECTRONIC SEQUENCE LISTING

The contents of the electronic sequence listing (BROD_4440_ST25.txt”; Size is 11,672 bytes and it was created on Sep. 19, 2019) is herein incorporated by reference in its entirety.

### TECHNICAL FIELD

The subject matter disclosed herein is generally directed to gene expression profiles and signatures of CAR T cells, compositions of CAR T cells and populations, methods and assays of CAR T cells, populations, treatments, and therapies, methods and assays of engineering and/or administering CAR T cells.

### BACKGROUND

Chimeric antigen receptors (CARs) are recombinant proteins useful redirect T lymphocytes and their functions. Tumor-targeted T cells can be rapidly generated and bypass immunization mechanisms. CARs with different strengths and signaling have the potential to modulate T-cell expansion and persistence as well as the strength of T-cell activation, characteristics that alter the efficacy and safety of tumor-targeted T cells.

T cells engineered to express chimeric antigen receptors (CARs) targeting CD19 have produced impressive outcomes for the treatment of B cell malignancies. CARs have been developed by independent groups and have incorporated different costimulatory domains, such as CD28 or 4-1BB. Both types of CAR constructs have demonstrated efficacy in B cell leukemias and lymphomas, but their engraftment kinetics, persistence and toxicity profiles are distinctive, with CD28-based CARs undergoing more rapid expansion with less persistence than 4-1BB-based CARs. Despite the tremendous momentum of CAR T cell development, an unbiased, broad and in-depth understanding of the functional state of different types of CAR-modified T cells is still limited.

CARs commonly contain 3 modules: an extracellular target binding module, a transmembrane domain (TM domain), and an intracellular signaling domain (ICD) that transmits activation signals (see Sadelain M. et al., Cancer Discov. 2013; 3(4):388-398). TM domains are primarily considered a structural requirement, anchoring the CAR in the cell membrane, and are most commonly derived from molecules regulating T cell function, such as CD8 and CD28. The intracellular module typically comprises a T cell receptor CD3ζ chain, often referred to as the “activator,” and one or more signaling domains such as from CD28, 4-1BB, OX40, CD27, or ICOS costimulatory proteins (see van der Stegen S J, et al., Nat Rev Drug Discov. 2015; 14(7):499-509). CARs containing either CD28 or 4-1BB costimulatory domains have been the most widely used, to date, and both of them have yielded dramatic responses in clinical trials (see Maude S L, et al. N Engl J Med. 2014; 371(16):1507-1517). Several studies suggest that the CD28 intracellular domain stimulates greater CAR T cell functionality, whereas the 4-1BB intracellular domain promotes greater CAR T cell persistence. See Guedan, S et al., JCI Insight. 2018; 3(1):e96976. However, the mechanisms by which different TM and intracellular domains influence T cell expansion, function, and persistence are not yet fully understood.

A molecular atlas of cells in the human body can (1) provide catalog cell types and subtypes (e.g., neurons, T-cells, etc.); (2) distinguish between cell states (e.g., a naïve immune cell compared to the same immune cell type after encountering a bacterium); (3) relate cell types to their position; (4) capture the salient characteristics of cells during transitions such as differentiation or activation; (5) chart interactions between cells and, when possible, (6) trace the history of cells through a lineage. Very recent advances in single-cell genomic analysis of cells and tissues have put within reach such a systematic, high-resolution effort to comprehensively characterize human cells. For example, massively parallel assays (e.g., Drop-Seq (Macosko et al., 2015, Cell 161, 1202-1214) and InDrop (Klein et al., 2015, Cell 161, 1187-1201) for RNA, (CyTOF for proteins) can now process tens and hundreds of thousands of cells at very low cost. Rapidly emerging experimental and computational techniques that couple molecular profiling of RNA or proteins with spatial information from sub-tissue to sub-cellular resolution (e.g., MERFISH (Chen et al., 2015, Science 348, aaa6090), FISSEQ (Lee et al., 2014, Science 343, 1360-1363), MIBI (Angelo et al., 2014, Nat Med 20, 436-442), Tomo-Seq (Junker et al., 2014, Cell 159, 662-675), Seurat (Satija et al., 2015, Nature biotechnology 33, 495-502) and more). Taken together, it is now possible to discover types, states, locations, transitions, interactions and lineage relations at an unprecedented resolution and scale. Building a Human Cell Atlas nevertheless poses substantial challenges that can be overcome only in the context of a joint concerted effort. Because cells of the same type may reside in multiple tissues (depending on how “type” is defined), we must be able to compare across tissues in consistent ways. Because of the large number and diversity of tissue and cell types, we must bring together biomedical expertise in tissue and cell types, genomic and engineering expertise in efficient, high-quality, scalable data production, and computational expertise in data analysis, engineering and visualization.

A Human Cell Atlas is akin to having the “zip code” of each cell type. It would provide foundational biological knowledge on the composition of multicellular organisms enabling us to understand how cell types weave together in three dimensions to form tissues, how the map is generated to connect all of the body's systems, and how changes in the map underlie health and disease. The atlas would also enable us to develop effective diagnostics and treatments. Specifically, it would: Serve as a reference map for discovery and characterization of cell types and states. An Atlas would identify new cell types, states, and transitions, as well as their molecular characteristics, thereby defining the functions of known and novel cell types. It would also help compare cells of similar types between contexts, such as tissues, health and disease state, different individuals, and between human and model organisms. Cell type expression profiles would also help determine the cell types in which disease-associated genes are acting. It can also help determine the cell types in which specific genes are expressed thus providing better guidance into the design of therapeutics (e.g. CAR-T) and better prediction of drug toxicities.

Citation or identification of any document in this application is not an admission that such document is available as prior art to the present invention.

### SUMMARY

In certain example embodiments, the invention provides a method of preparing a candidate CAR T cell or a population of CAR T cells or enhancing the presence of a candidate CAR T cell in a population of CAR T cells. In another aspect, the invention provides a method of evaluating a CART cell or population for the presence of a candidate CART cell. In another aspect, the invention provides a method of examining a patient or patient population to assess suitability for a CAR T cell treatment or therapy. In another aspect, the invention provides a method of matching or improving the suitability of a CAR T cell treatment or therapy with a patient or patient population. In certain embodiments, the CAR T cell comprises a CD4⁺ CAR T cell. In certain embodiments, the CAR T cell comprises a CD8⁺ CAR T cell. By CD4⁺ or CD8⁺ CAR T cell is meant a CAR T cell that expresses CD4 or CD8 respectively. In certain embodiments, the CAR T cell comprises a T-helper cell subset, for example without limitation, a T_{H}1 CAR T cell, or a T_{H}2 CAR T cell. Besides the classical T-helper 1 and T-helper 2, other subsets include T-helper 17, regulatory T cells (Treg), follicular helper T cells, and T-helper 9, each with a characteristic cytokine profile. For example, a T_{H}1 CAR T cell can be descripted as a CD4⁺ cell that promotes a T_{H}1 response and a T_{H}2 CAR T cell can be descripted as a CD4⁺ cell that promotes a T_{H}2 response.

In general, the method involves measuring expression of one or more signature genes of a CAR T cell. In an embodiment, the method involves measuring expression of one or more signature genes of a CAR T cell and identifying the CAR T cell as a candidate if the cell upregulates one or more signature genes that are upregulated in a CD3ζ CAR T cell and/or downregulates one or more signature genes that is downregulated in a CD3ζ CAR T cell. In certain embodiments, the CD3ζ CAR T cell comprises a BBζ CAR T cell, i.e., the CAR construct in the CAR T cell comprises both a CD3ζ activation domain and a BB1-44 stimulatory domain. In certain embodiments, the CD3ζ CAR T cell comprises a 28ζ CAR T cell, i.e. the CAR construct in the CAR T cell comprises both a CD3ζ activation domain and a CD28 stimulatory domain.

In certain example embodiments, provided herein are methods of identifying a candidate CAR T cell comprising: measuring expression of a gene signature of a CAR T cell and identifying the CAR T cell as the candidate CAR T cell if the CAR T cell a gene signature selected from:

a) a CD3ζCAR T gene signature,

b) a costimulatory molecule gene signature,

c) a T_{H}1 response gene signature,

d) a T_{H}2 response gene signature,

e) a T cell activation gene signature,

f) any combination thereof.

In certain example embodiments, the CD3ζ CAR T gene signature comprises one or more signature genes selected from the group consisting of: ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof. In certain example embodiments, one or more signature genes in the CD3ζ CAR T gene signature are up-regulated, down-regulated, or both. In certain example embodiments, the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises ZP3 or GGT1. In certain example embodiments, the CD3ζ CAR T gene signature comprises CCL3, CCL4, GZMB, XCL1, ZBED2, IFNG, or any combination thereof.

In certain example embodiments, the costimulatory molecule gene signature comprises one or more signature genes of Table 7, Table 8, or any combination thereof. In certain example embodiments, one or more signature genes in the costimulatory molecule gene signature are up-regulated, down-regulated, or both. In certain example embodiments, the costimulatory molecule gene signature comprises a gene signature selected from the group consisting of:

(a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6, ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
(b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
(c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, Clorf228, DNAJC5B, SLC25A22, BCL2L11, RPL21P28, TMOD1, CDKN2A, LRP8, MLLT4, ADAP1, JAK1, IFI44, MROH8, and any combination thereof;
(d) JUN, GPA33, KRT1, EGR1, CIITA, UBD, KLHL23, SCD, HLA-DOA, ALPK, CXCL10, and any combination thereof;
(e) JUN, EGR1, CIITA, GPA33, KRT1, and any combination thereof;
(f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
(g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
(h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
(i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
(j) CXCL10, JUNB, NTRK2, MSC, VNN2 and any combination thereof;
(k) JUNB, CXCL10, ENOX1, ENPP2, DDIT4, NTRK2, GCSAM, IL5, and any combination thereof;
(l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
(m) ENPP2, GJB2, C4orf26, MX1, NTRK2, JUNB, TNFRSF8, DGAT2, ELL2, IL4I1, ITPR1, HLA-DRB6, and any combination thereof; and
(n) CIITA, CD74, HLA-DMB, HLA-DPB1, HLA-DQA2, HLA-DRB1, HLA-DRB5, HLA-DOA, HLA-DRA, HLA-DRB6, and any combination thereof.

In certain example embodiments, the CAR T cell is CD4+. In certain example embodiments, the gene signature is any one of gene signatures (a)-(i).

In certain example embodiments, the CAR T cell is CD8+. In certain example embodiments, the gene signature is any one of gene signatures (a), (b), (c), (j), (k), (l), or (m).

In certain example embodiments, the CAR T cell is unstimulated. In certain example embodiments, the gene signature is any one of gene signatures (a), (d), (e), or (j).

In certain example embodiments, the CAR T cell is stimulated. In certain example embodiments, the gene signature is any one of gene signatures (b), (c), (f), (g), (h), (i), (k), (l), or (m).

In certain example embodiments, the CAR T cell expresses a CD28ζ co-stimulatory molecule. In certain example embodiments, one or more genes in any one of gene signatures (a)-(i) is up-regulated, down-regulated, or both as compared to a CAR T cell expressing a BBζ co-stimulatory molecule. In certain example embodiments, LGMN, PDCD1, GPA33, KRT1, VNN2, C17orf-PLSCR3, and any combination thereof is up-regulated in the CART cell as compared to a CAR T expressing a BBζ co-stimulatory molecule. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is down-regulated in the CART cell as compared to a CAR T expressing a BBζ co-stimulatory molecule.

In certain example embodiments, the CAR T cell expresses a BBζ co-stimulatory molecule. In certain example embodiments, one or more genes in any one of gene signatures (a)-(i) is up-regulated, down-regulated, or both as compared to a CAR T cell expressing a CD28ζ co-stimulatory molecule. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is up-regulated in the CAR T cell. In certain example embodiments, LGMN, PDCD1, GPA33, KRT1, VNN2, C17orf-PLSCR3, and any combination thereof is down-regulated in the CART cell.

In certain example embodiments, the T_{H}1 response gene signature comprises one or more signature genes selected from the group consisting of: ERG1, TBX21, RORC, IL12RB2, GLIL1, EPPN2, DMD, IFNG, and any combination thereof. In certain example embodiments, the CAR T cell expresses a BBζ co-stimulatory molecule. In certain example embodiments, the CAR T cell is CD4+.

In certain example embodiments, the T_{H}2 response gene signature comprises one or more signature genes selected from the group consisting of: IL4, IL5, IL2, and any combination thereof. In certain example embodiments, the CAR T cell expresses a CD28ζ co-stimulatory molecule. In certain example embodiments, the CAR T cell is CD4+.

In certain example embodiments, the T cell activation gene signature comprises one or more genes selected from Table 3, Table 4, or a combination thereof.

In certain example embodiments, the T cell activation gene signature comprises one or more genes selected from the group consisting of: IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof. In certain example embodiments, the stimulated CAR T cell was generated by stimulating the CAR T cell through a T cell receptor of the CAR T cell.

In certain example embodiments, IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are upregulated as compared to a CAR T cell stimulated through a CAR of the CAR T cell.

In certain example embodiments, the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1

TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

In certain example embodiments, the CAR T is a stimulated CAR T cell, wherein the stimulated CAR T cell was generated by stimulating a chimeric antigen receptor of CAR T cell.

In certain example embodiments, measuring expression of a gene signature comprises bulk RNA sequencing, single cell RNA sequencing (scRNA-seq), or both.

In certain example embodiments, the method further comprises isolating an identified candidate CAR T cell or a population thereof to obtain an isolated candidate CAR T cell or population thereof and optionally expanding the isolated candidate CAR T cell or population thereof to obtain an expanded candidate CAR T cell or population thereof.

In certain example embodiments, the method further comprises administering the isolated candidate CAR T cell or population thereof or the expanded candidate CAR T cell or population thereof to a subject in need thereof. In certain example embodiments, the subject in need thereof has a cancer.

In certain example embodiments, provided herein are methods of modulating a CAR T cell, comprising: administering a modulating agent to a CAR T cell, wherein the modulating agent is capable of modifying the expression of one or more genes in the CAR T cell such that the CAR T cell comprises a gene signature selected from:

a) a CD3ζ CAR T gene signature,

b) a costimulatory molecule gene signature,

c) a T_{H}1 response gene signature,

d) a T_{H}2 response gene signature,

e) a T cell activation gene signature,

f) any combination thereof.

In certain example embodiments, the CD3ζ CAR T gene signature comprises one or more signature genes selected from the group consisting of: ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof. In certain example embodiments, one or more signature genes in the CD3ζ CAR T gene signature are up-regulated, down-regulated, or both. In certain example embodiments, the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises ZP3 or GGT1. In certain example embodiments, the CD3ζ CAR T gene signature comprises CCL3, CCL4, GZMB, XCL1, ZBED2, IFNG, or any combination thereof.

In certain example embodiments, the costimulatory molecule gene signature comprises one or more signature genes of Table 7, Table 8, or any combination thereof. In certain example embodiments, one or more signature genes in the costimulatory molecule gene signature are up-regulated, down-regulated, or both. In certain example embodiments, the costimulatory molecule gene signature comprises a gene signature selected from the group consisting of:

(a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6, ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
(b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
(c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, Clorf228, DNAJC5B, SLC25A22, BCL2L11, RPL21P28, TMOD1, CDKN2A, LRP8, MLLT4, ADAP1, JAK1, IFI44, MROH8, and any combination thereof;
(d) JUN, GPA33, KRT1, EGR1, CIITA, UBD, KLHL23, SCD, HLA-DOA, ALPK, CXCL10, and any combination thereof;
(e) JUN, EGR1, CIITA, GPA33, KRT1, and any combination thereof;
(f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
(g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
(h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
(i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
(j) CXCL10, JUNB, NTRK2, MSC, VNN2 and any combination thereof;
(k) JUNB, CXCL10, ENOX1, ENPP2, DDIT4, NTRK2, GCSAM, IL5, and any combination thereof;
(l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
(m) ENPP2, GJB2, C4orf26, MX1, NTRK2, JUNB, TNFRSF8, DGAT2, ELL2, IL4I1, ITPR1, HLA-DRB6, and any combination thereof, and
(n) CIITA, CD74, HLA-DMB, HLA-DPB1, HLA-DQA2, HLA-DRB1, HLA-DRB5, HLA-DOA, HLA-DRA, HLA-DRB6, and any combination thereof.

In certain example embodiments, the gene signature is any one of gene signatures (a)-(i). In certain example embodiments, the gene signature is any one of gene signatures (a), (b), (c), (j), (k), (l), or (m). In certain example embodiments, the gene signature is any one of gene signatures (a), (d), (e), or (j). In certain example embodiments, the gene signature is any one of gene signatures (b), (c), (f), (g), (h), (i), (k), (l), (m). In certain example embodiments, one or more genes in any one of gene signatures (a)-(i) is overexperssed, underexpressed, or both as compared to an unmodified CAR T cell. In certain example embodiments, LGMN, PDCD1, GPA33, KRT1, VNN2, C17orf-PLSCR3, and any combination thereof is overexpressed in the CART cell. IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is underexpressed in the CART cell. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is overexpressed in the CAR T cell. In certain example embodiments, LGMN, PDCD1, GPA33, KRT1, VNN2, C17orf-PLSCR3, and any combination thereof is underexpressed in the CART cell.

In certain example embodiments, the T_{H}1 response gene signature comprises one or more signature genes selected from the group consisting of: ERG1, TBX21, RORC, IL12RB2, GLIL1, EPPN2, DMD, IFNG, and any combination thereof.

In certain example embodiments, the T_{H}2 response gene signature comprises one or more signature genes selected from the group consisting of: IL4, IL5, IL2, and any combination thereof.

In certain example embodiments, the T cell activation gene signature comprises one or more genes selected from Table 3, Table 4, or a combination thereof.

In certain example embodiments, the T cell activation gene signature comprises one or more genes selected from the group consisting of: IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof. In certain example embodiments, IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are overexpressed or underexpressed in the CAR T cell. In certain example embodiments, the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1 TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

In certain example embodiments, the modifying agent is a therapeutic antibody, antibody fragment, antibody-like protein scaffold, aptamer, polypeptide, protein, genetic modifying agent, small molecule, small molecule degrader, or combination thereof. In certain example embodiments, the genetic modifying agent is a CRISPR-Cas system, a TALEN, a Zn-finger nuclease, or a meganuclease.

In certain example embodiments, provided herein are isolated or engineered CAR T cells obtained according to any method described herein such as those in numbered aspects 1-72.

In certain example embodiments, provided herein are methods of treating a disease in a subject in need thereof comprising: administering an identified candidate cell obtained by the method as in any one of numbered aspects 1-44 or an isolated or engineered CAR T cell as in numbered aspect 73, or a cell population thereof to the subject. In certain example embodiments, the disease is a cancer. In certain example embodiments, the method can further comprise administering an additional agent, therapy, antineoplastic or antitumor agent or radiation and/or surgical therapy or an antigen or a neoantigen. In certain example embodiments, the additional agent, therapy, antineoplastic or antitumor agent or radiation and/or surgical therapy or an antigen or neoantigen is administered sequentially or concurrently. In certain example embodiments, the sequential administration comprises a time period of a day, two days, three days, four days, five days, six days, a week, two weeks, three weeks, four weeks, five weeks, six weeks, seven weeks, eight weeks, nine weeks, ten weeks, eleven weeks, twelve weeks, or more.

In certain example embodiments, provided herein are methods of screening for one or more agents capable of modifying a gene expression signature of a CAR T cell as in any one of numbered aspects 45-72, comprising: contacting an unmodified CAR T cell population with a test modulating agent or a library of modulating agents; identifying candidate CAR T cells present in the CART T cell population by the method of any one of numbered aspects 1-44; and selecting modulating agents that result in increasing the number of candidate CAR T cells present in the CAR T cell population.

In certain example embodiments, the CAR T cell or population thereof is obtained from or derived from a subject to be treated.

In certain embodiments, expression of two, three, four, five, six, seven, eight, nine, ten, or more signature genes are measured. In certain embodiments, the signature genes are those of Table 7—CAR T Gene Expression Data herein. In certain embodiments, the signature genes comprise one, two, three, four, five, six, seven, eight, nine, ten, or more signature genes of ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, OR HPCAL1.

In an embodiment, the method involves measuring expression of one or more signature genes of a CAR T cell and identifying the CAR T cell as a candidate CD4⁺ CAR T cell if the cell upregulates one or more signature genes that are upregulated in a BBζ CAR T and/or downregulates one or more signature genes that is downregulated in a BBζ CAR T cell.

In an embodiment, the method involves measuring expression of one or more signature genes of a CAR T cell and identifying the CAR T cell as a candidate CD8⁺ CAR T cell if the cell upregulates one or more signature genes that are upregulated in a 28ζ CAR T and/or downregulates one or more signature genes that is downregulated in a 28ζ CAR T cell.

In certain embodiments, expression of two, three, four, five, six, seven, eight, nine, ten, or more signature genes are measured. In certain embodiments, the signature genes are those of Table 1 of US Pat. App. Pub. 2019/0255107. In certain embodiments, the signature genes comprise one, two, three, four, five, six, seven, eight, nine, ten, or more signature genes of GJB2, UBD, NTRK2, THY1, HLA-DQA2, G0S2, CXCL10, DOHH, MSC, DMD, HLA-DOA, ANXA3, FILIP1L, EMC8, SH3B5, HLA-DRB1, JUNB, CDK6, ACSL1, HLA-DRB5, HLA-DRB6, ANK3, MPZL1, IFNG, NOD2, TMEM165, LGMN, or PDCD1.

In certain embodiments, the candidate is a candidate CD4⁺ CAR T cell, the one or more signature genes comprises ZP3 or GGT1. In certain embodiments, the candidate is a candidate CD8⁺ CAR T cell, the one or more signature genes comprises CCL3, CCL4, GZMB, XCL1, ZBED2, or IFNG.

In an embodiment, the method involves measuring expression of one or more signature genes of a CAR T cell and identifying the CAR T cell as a candidate CD4⁺ CAR T cell that promotes a T_{H}1 response if the cell upregulates one or more signature genes that are upregulated in a BBζ CAR T that promotes a T_{H}1 response and/or downregulates one or more signature genes that is downregulated in a BBζ CAR T cell that promotes a T_{H}1 response. In certain embodiments, the signature genes comprise those identified for CD4⁺ CAR T cells and further comprise one, two, three, four, five, six, seven, or eight of EGR1, TBX21, RORC, IL12RB2, GLUL1, EPPN2, DMD, or IFNG.

In an embodiment, the method involves measuring expression of one or more signature genes of a CAR T cell and identifying the CAR T cell as a candidate CD4⁺ CAR T cell that promotes a T_{H}2 response if the cell upregulates one or more signature genes that are upregulated in a BBζ CAR T that promotes a T_{H}2 response and/or downregulates one or more signature genes that is downregulated in a BBζ CAR T cell that promotes a T_{H}2 response. In certain embodiments, the signature genes comprise those identified for CD4⁺ CAR T cells and further comprise one, two, or three of IL2, IL4, or IL5.

In some instances, a candidate cell can be deficient for or overexpress a signature gene or be deficient in some other respect. For example, a candidate cell may be identified by one or more signature gene profiles and/or other cell characteristics but not upregulate or downregulate one or more other signature genes of the profile or be deficient for expression of a phenotype desired of an appropriate CAR T cell. Accordingly, the invention provides for modification of CAR T cells or their precursor cells used to make a CAR T cell. In certain embodiments, preparing a CAR T cell further comprises engineering the CAR T cell to modulate expression of a signature gene or gene product or other characteristic, or administering an agent with the CAR T cell that modulates expression of a signature gene or gene product or other characteristic.

In an aspect, the invention provides a method of identifying suitability of a CAR T cell or CAR T cell population for administration to a mammalian subject. In certain embodiments, the mammal is a human. In certain embodiments, the mammal is a non-human primate. In certain embodiments, the subject is a laboratory animal. In certain embodiments, the subject is a domesticated animal. In certain embodiments, the subject is a farm animal or livestock. Non-limiting examples include a rodent, e.g., mouse, rat, gerbil, hamster, Leporidae, e.g., rabbit, a feline, e.g., cat, tiger, lion, canine, e.g., dog, porcine, e.g., pig, piglet, sow, boar, gilt, bovine, e.g., cattle, bison, Equidae, e.g., horse, donkey, primate, e.g., chimpanzee, gorilla, ape, orangutan, baboon, macaque, or human.

The invention provides for an assessment of expansion or administration. In addition to genetic makeup, suitability for administration to a subject can depend, e.g., on age and/or sex of the subject. Suitability for expansion of an immune cell or precursor can also depend on age. For example, it is understood that immune systems and components, including immune cells and cell populations change over time and with age, for example that immune responses in humans differ among neonates, children, young adults, middle age adults, seniors, and geriatric adults. Accordingly, in certain embodiments, suitability is assessed for a male, a female, an adult, e.g., post puberty or having secondary sexual characteristics or at least age 13, 14, 15, 16, 17, 18, 19, 20 or 21, or an older middle age or senior or geriatric adult (e.g., at least age 50, 55, 60, 65, 70, 75, 80, 85) or young adult at least age 13, 14, 15, 16, 17, 18, 19, 20 or 21 to about age 30 or 35, or middle age adult e.g., an adult older than a young adult and younger than an older middle age or senior or geriatric adult, or child, e.g., pre puberty or pre secondary sexual characteristics or pediatric individual or less than age 13, 14, 15, 16, 17, 18, 19, 20 or 21.

In certain embodiments, additional aspects relating to immune cells and system function are determined and/or modified. For example, it will be advantageous to measure and/or correct expression of signatures genes relating to immune cell or system function, for example, that can vary from subject to subject or be defective in a subset of subsets, and which may be consequences of mutation, subject age, treatment history and the like. The invention includes compositions to measure or confirm or correct gene regulation and/or gene product activity of a CAR T cell or a population of CAR T cells or patient cells used to make CAR T cells.

An aspect of the invention provides a method of detecting dysfunctional immune cells comprising detection of a gene expression signature of dysfunction selected from the group consisting of: a) a signature comprising or consisting of one or more markers selected from the group consisting of CD83, CCR8, TNFRSF4, CD74, CCR7, TNFSF11, CD81, TBC1D4, REL, PLK2, XCL1, TNFSF4, SLC2A6, AI836003, LAD1, 1700019D03RIK, BCL6, MNDA, RAMP3, GPM6B, BHLHE40, AXL, ECE1, FILIP1L, KIT, ITGB1, CCL1, NFKB2, PLXDC2, ARC, DUSP4, CD200, TRAF1, ZHX2, NCF1, CCDC28B, PTPRS, ST6GALNAC3, TUSC3, PDCD1LG2, SDHAF1, ARAP2, KLF4, E130308A19RIK, FAM46A, TNFRSF18, SYNJ2, CYTH3, TNFSF8, CD160, RPL10, CRTAM, RAB6B, PTGER2, NFKB1, ANKRD46, ST6GALNAC6, ITPR1, ITM2C, BTLA, TSPAN32, CD82, NFKBIA, MS4A4C, RARG, NRGN, TRIB1, ZC3H12D, BMYC, IFI27L2A, GADD45B, NAPSA, KLRB1F, RASGEF1A, FOSB, MAP3K8, HIVEP1, SSH1, RABGAP1L, ZFP36L1, ARL4D, CACNA1S, NFAT5, DNAJC12, SOWAHC, SDF4, TMEM120B, DUSP1, ELK3, JUNB, GRAMD1B, LIMK1, ZFC3H1, OSTF1, LTA, DNMT3A, BCL7C, TSPAN13, ASNSD1, TGIF1, NRN1, SYNGR2, MSI2, UAP1, UNC93B1, JAK2, KDM2B, ANXA5, PRDX2, TMEM173, PHACTR2, CCDC104, CEP85L, IRF5, INF2, ITGB3, MPC1, BCL2A1D, PARP3, ASAP1, MRPS6, RELB, FAM110A, GPR68, NRP1, CAPG, SCYL2, SAMD3, H2-AB1, HSF2, CD44, STX6, POLG2, TESPA1, ALCAM, NSMF, LRRC8D, HIF1A, PACSIN1, PKP4, ASS1, NR4A3, ENO3, GYPC, KIF3B, IL2RA, RAB37, SGMS1, HLCS, SEMA6D, NMRK1, SLC17A6, SLC39A1, RPS4X, CDON, ZFP445, LAG3, RPS26, PHTF2, CST3, CD9, STAT5A, ABCA3, CSF2RA, DTX3, RSPH3A, NRIP1, SDHA, PNKD, FLNB, MGRN1, SLC26A2, HMOX2, PEX16, INPP4A, TNFRSF25, IRF8, RGCC, IFITM2, TNFSF14, NSUN6, STAT3, PFKFB3, TYROBP, HTRA2, KLRI2, CTSS, ARL5C, KLHL24, SESN3, GM5424, FAS, NCOA3, FAM53B, CALCOCO1, ERGIC3, 4930523C07RIK, PCGF5, ANXA4, and HERPUD1; b) a signature comprising or consisting of one or more markers selected from the group consisting of CD74, CCR7, TBC1D4, SLC2A6, BCL6, JAK2, PARP3, ASAP1, RELB, H2-AB1, CD44, ABCA3, PFKFB3, SESN3, FAS, 4930523C07RIK, PCGF5, TNIP1, SPRY1, NCOA7, RPLP0, SMIM8, ANTXR2, NSMCE1, DEDD, B3GNT2, CABLES1, SLAMF6, UBL3, NR4A1, ATG7, and KDM5B; c) a signature comprising or consisting of one or more markers selected from the group consisting of CD83, CCR8, TNFRSF4, CD74, CCR7, TNFSF11, CD81, XCL1, TNFSF4, AXL, ECE1, KIT, ITGB1, CCL1, CD200, TNFRSF18, TNFSF8, CD160, PTGER2, BTLA, TSPAN32, CD82, KLRB1F, LTA, ANXA5, ITGB3, NRP1, H2-AB1, CD44, ALCAM, GYPC, IL2RA, CDON, LAG3, CD9, TNFSF14, FAS, GDI2, TNIP1, IL21R, IL18R1, H2-AA, NR4A2, IL18RAP, CD97, TNFSF9, IRAK1BP1, GABARAPL1, TRPV2, EBAG9, GRN, RAMP1, AIMP1P, BSG, IFNAR1, PRKCA, TRAF3, CD96, TNFRSF9, and NR3C1; d) a signature comprising or consisting of one or more markers selected from the group consisting of CD83, TNFRSF4, CD74, CCR7, CD81, TNFSF4, KIT, ITGB1, CD200, TNFSF8, CD160, CD82, ITGB3, CD44, ALCAM, GYPC, IL2RA, CDON, LAG3, CD9, CSF2RA, FAS, CD97, TNFSF9, BSG, IFNAR1, TRAF3, CD96, and TNFRSF9;

e) a signature comprising or consisting of one or more markers selected from the group consisting of CD83, CD81, TNFRSF4, CXCL16, IL21R, and IL18R1; f) a signature comprising or consisting of one or more markers selected from the group consisting of REL, BCL6, MNDA, BHLHE40, NFKB2, ZHX2, KLF4, NFKB1, NFKBIA, RARG, FOSB, HIVEP1, ZFP36L1, NFAT5, ELK3, JUNB, LIMK1, TGIF1, KDM2B, IRF5, RELB, HSF2, HIF1A, NR4A3, PHTF2, STAT5A, DTX3, NRIP1, IRF8, STAT3, NCOA3, CALCOCO1, PCGF5, NFKBIE, ETV6, RNF19A, STAT4, NR4A2, NFKBIB, PER1, GTF2A1, SPRY1, TFE3, TGIF2, RORA, RPL6, EGR2, FOXP4, TBL1X, KDM4A, COPS2, FOS, DEDD, SQSTM1, NT5C, PIAS4, ZMYM2, DMTF1, AEBP2, TRPS1, SP3, HBP1, NR4A1, TLE3, RPL7, MED21, DRAP1, TCF7, CREB3L2, ZFHX2, KDM5B, and NR3C1; or g) a signature comprising or consisting of two or more markers each independently selected from any one of the groups as defined in any one of a) to f).

In certain embodiments, the signature further comprises one or more additional markers of dysfunction. In an embodiment, the one or more additional markers of dysfunction is a co-inhibitory receptor selected from the group consisting of PD1, CTLA4, TIGIT, TIM3, LAG3, KLRC1, BTLA, NRP1, CD160, CD274, IDO, CD200, CD244, KLRD1, LAIR1, CEACAM1, KLRA7, FAS, GPR132, CD74, SLAMF6, CD5, GPR35, CD28, CD44, and PTGER4.

Another aspect of the invention involves IL-27 signaling that drives the expression of a gene module that includes not only Tim-3, but also Lag-3, TIGIT, and IL-10, all molecules that are associated with T cell dysfunction. The IL-27-induced transcriptional module significantly overlaps with the gene signatures that define dysfunctional T cells in chronic viral infection and cancer, as well as with gene signatures associated with other suppressed or tolerant T cell states.

Another aspect of the invention involves altering FAS-STAT1 binding. In certain embodiments, the T cell is modified to express a recombinant polypeptide capable of antagonizing FAS-STAT1 interaction. In certain embodiments, the polypeptide does not affect the binding of FAS to FAS-L. In certain embodiments, the polypeptide does not affect the binding of FAS to FADD.

In certain embodiments, the T cell is modified to express a recombinant polypeptide that is capable of adopting a FAS ligand bound conformation, is inactivated for apoptotic signaling, and is able to bind to STAT1. Thus, the recombinant polypeptide is only able to antagonize FAS-STAT1 binding. In certain embodiments, the polypeptide does not affect the binding of FAS to FAS-L. In certain embodiments, the polypeptide does not affect the binding of FAS to FADD.

In certain embodiments, the T cell is modified to over-express STAT1. Thus, in certain embodiments, increased expression of STAT1 can saturate binding to FAS and shift T cell balance towards a Th1 phenotype.

In certain embodiments, the T cell is modified to abolish or knockdown expression or activity of STAT1 and is differentiated under Th17 conditions. The Th17 conditions may comprise cultures supplemented with IL-6 and TGF-β1 or supplemented with IL-1β, IL-6 and IL-23. The T cell may comprise a genetic modifying agent targeting STAT1. The genetic modifying agent may comprise a CRISPR system, a zinc finger nuclease system, a TALEN, or a meganuclease. The CRISPR system may comprise Cas9 or Cpf1 and target the STAT1 gene. The CRISPR system may comprise a Cas13 system and target STAT1 mRNA. The Cas13 system may comprise Cas13-ADAR.

In certain embodiments, the T cell is modified to comprise a non-silent mutation in FAS and/or STAT1, wherein the mutation inhibits FAS-STAT1 binding. The mutation may alter a post-translational modification site in FAS and/or STAT1 that alters FAS-STAT1 binding. The mutation may not inhibit FAS apoptotic signaling. The T cell may comprise a genetic modifying agent targeting FAS and/or STAT1. The genetic modifying agent may comprise a CRISPR system, a zinc finger nuclease system, a TALEN, or a meganuclease. The CRISPR system may comprise a Cas13 system and target FAS and/or STAT1 mRNA. The Cas13 system may comprise Cas13-ADAR.

In certain embodiments, the T cell is modified to decrease, but not eliminate expression or activity of FAS. The T cell may be differentiated under Th17 conditions. The Th17 conditions may comprise cultures supplemented with IL-6 and TGF-β1 or supplemented with IL-1β, IL-6 and IL-23. The T cell may comprise a genetic modifying agent targeting FAS. The genetic modifying agent may comprise a CRISPR system, a zinc finger nuclease system, a TALEN, or a meganuclease. The CRISPR system may comprise a Cas13 system and target FAS mRNA. The Cas13 system may comprise Cas13-ADAR.

In certain embodiments, the isolated T cell of any embodiment is a Th17 cell. In certain embodiments, the T cell is a naïve Th0 cell. In certain embodiments, the T cell is a tumor infiltrating lymphocyte (TIL). In certain embodiments, the T cell expresses an endogenous T cell receptor (TCR) or chimeric antigen receptor (CAR) specific for a tumor antigen. In certain embodiments, the T cell is expanded. In certain embodiments, the T cell is modified to express a suicide gene, wherein the modified T cell can be eliminated upon administration of a drug.

The invention further includes ILT-3 and novel ILT-3 ligands CD 166, angiopoetins, and angiopoetin-like proteins as important co-stimulatory and co-inhibitory receptors of T cells. A method of modulating T cell dysfunction is provided, the method comprising contacting a dysfunctional T cell with a modulating agent or agents that modulate the expression, activity and/or function of ILT-3. In another embodiment of this aspect, the modulating agent promotes the expression, activity and/or function of the ILT-3 gene or gene product or combination thereof. In another embodiment of this aspect the modulating agent inhibits the expression, activity and/or function of the ILT-3 gene or gene product or combination thereof.

In another embodiment of this aspect, the modulating agent inhibits binding of ILT-3 to one or more ILT-3 ligands. In another embodiment, the one or more ILT-3 ligands is selected from integrin αvβ3, CD 166, ANGPT1, ANGPT2, ANGPT3, ANGPT4, ANGPTL1, ANGPTL2, ANGPTL3, ANGPTL4, ANGPTL5, ANGPTL6, ANGPTL7, and ANGPTL8. In another embodiment, the modulating agent promotes or inhibits the expression, activity and/or function of one or more genes selected from ANGPT1, ANGPT2, ANGPT3, ANGPT4, ANGPTL1, ANGPTL2, ANGPTL3, ANGPTL4, ANGPTL5, ANGPTL6, ANGPTL7, and ANGPTL8 or gene products thereof or combinations thereof.

Another aspect of the invention relates to checkpoint blockade therapy signature genes TCF7, LEF1, S1PR1, PLAC8, LTB, CCR7, IGHD, PAX5, FCRL1, FCER2, CD19, CD22, BANK1, MS4A1, BLK, RALGPS2 and FAM129C; or TCF7, PLAC8, LTB, LY9, SELL, IGKC and CCR7 and further checkpoint blockade (CPB) therapy non-responder signature genes identified herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1I. Antigen stimulation of CAR T cells through their CAR yields a weaker but similar T cell activation signal compared to stimulation via anti-CD3-TCR. FIG. 1A) Vector maps of CD19 CAR constructs. TM, hinge and transmembrane domain. L, leader sequence. FIG. 1B) Experimental design of bulk RNA sequencing. T cells were isolated from a leukopak and sorted on CD3⁺, CD8⁺ or CD4⁺ then mixed at 1:1 CD4-to-CD8 ratio, activated with anti-CD3/CD28 beads and transduced with one of four constructs or left untransduced (UT). Cells were expanded for 7 days then beads were removed, and cells were rested for 7 days prior to reactivation with anti-CD3/anti-CD28 beads or irradiated Nalm6 cells for 4 or 24 hours. Samples were then sorted on CAR⁺ CD4⁺ or CD8⁺ T cells and sequenced separately as bulk populations. Data were collected for T cells from three human donors with technical duplicates. FIG. 1C) Experimental design of single cell RNA sequencing. T cells were isolated, activated with anti-CD3/CD28 beads and transduced with one of three functional CAR constructs or left UT. Cells were expanded for 7 days then beads were removed. Cells were rested for 7 days prior to reactivation with irradiated Nalm6 cells for 24 hours. In addition, the UT T cell sample was also stimulated with irradiated K562 cells expressing an scFv against CD3. Stimulated and unstimulated samples were then sorted as live mCherry⁺ and then sequenced using 10× technology. More than 3,500 cells were sequenced per sample from two human donors. FIGS. 1D-1F) Principal component analysis of the expression profiles from the bulk RNAseq samples corrected for donor variation. Shown left to right by FIG. 1D) stimulation condition, FIG. 1E) CD4⁺ vs CD8⁺, and FIG. 1F) CAR construct. FIG. 1G) t-Distributed stochastic neighbor embedding (tSNE) of 83,123 single cell expression profiles (dots) as in plot of all single cells sequenced from two donors after donor-specific batch correction. Cells are labeled by their CAR (color hue) and the type of stimulation (light tones for unstimulated). FIG. 1H) tSNE expression profiles as in FIG. 1G, shown by the degree of CD8A and CD4 expression. FIG. 1I) Violin plots showing the distribution of T cell activation signature scores across each condition in CD4⁺ or CD8⁺ T cells. T cell activation gene signature defined as the 16 most upregulated genes CAR activated CAR T cells and anti-CD3 activated UT cells compared to resting T cells (gene signature in Table 3). *p<2.2*10⁻¹⁶, Wilcoxon test.

FIGS. 2A-2G. Transcriptional signatures of resting CAR T cells indicate tonic signaling through both CD3ζ and the co-stimulatory domain. FIG. 2A) Heat map of normalized expression from bulk RNA-Seq of both the up and downregulated genes (rows) that were differentially expressed (DE) in all three functional CARs: 28ζ, BBζ and ζ compared to Δζ at rest. The signature of CD3ζ chain tonic signaling was defined as the overlapping genes DE in all functional CARs vs. Δζ. FIG. 2B) tSNE of all CAR T cultures at rest, shown by the cluster assigned using a graph-based clustering approach. Assigned clusters were then defined based on the gene expression of several key genes. CD62L (SELL) levels were defined using average gene expression log fold change (log FC) with respect to the cluster with the highest CD62L expression (cluster 3). CD62L^{hi} (log FC<0.25), CD62L^{mid} (0.25<log FC<0.5 and adj-p<1e-50-Wilcox test) and CD62L^{low} (log FC>0.5, adj-p<1e-50-Wilcox test) FIG. 2C) The same tSNE plots as in FIG. 2B, depicting degree of CD4 and CD8A expression. FIG. 2D) Violin plots of cells showing the degree of expression of SELL and CCR7 used to describe the clusters shown in FIG. 2B. FIG. 2E) Violin plots of cells as clustered in B, showing degree of S phase and G2M phase signatures. FIG. 2F) The percent contribution of each CAR to a cluster. FIG. 2G) Graphical display showing the correlation matrix of the chi-square test residual values (difference between observed and expected values).

FIGS. 3A-3I. CAR T cells with the 4-1BB co-stimulation domain have persistent upregulation of markers associated with activation, particularly MHC Class II genes but not PD1. FIG. 3A) Volcano plots of differentially expressed genes between BBζ (positive x axis) and 28ζ CARs at 24 hours post-CAR activation in CD4⁺ (left) and CD8⁺ (right) T cells. Genes with FDR<0.05 are shown. FIG. 3B) Heat map showing normalized HLA II gene expression of CD4⁺ T cells from all three donors in 28ζ and BBζ CARs 24 hours post-CAR stimulation. FIGS. 3C-3E) Mean fluorescence intensity (MFI) of HLA-DR surface expression measured by flow cytometry on CD19-CAR T cells FIG. 3C) at rest or FIG. 3D) 24 hours after activation of CD19-specific CARs, or FIG. 3E) EGFR (U87)-mediated activation of EGFR-specific CAR T cells. FIG. 3F) IL21R expression in bulk RNA-seq BBζ and 28ζ CAR T samples with CAR stimulation. Individual TPM values shown with mean and SEM, adj-p values were calculated by DEseq2 using Holm-Bonferroni correction. FIG. 3G) IL-21 cytokine levels measured in the supernatants of bulk CD4⁺/CD8⁺ CD19-CAR T cells stimulated for 24 hours with Nalm6 cells or FIG. 311) EGFR-CAR T stimulated with U87 cells. FIG. 3I) MFI of PD1 expression in CD19-CAR T cells with CAR stimulation (via Nalm6 cells). N=3 normal donors, mean and SEM plotted. p-values were determined using a paired student t-test between BBζ and 28ζ, *p<0.05 **p<0.01 (FIGS. 3G-3I) or when required, adjusted for multiple comparisons using Holm-Bonferroni method adjustment (FIGS. 3C-3E). *adj-p<0.05**adj-p<0.01.

FIGS. 4A-4D. Antigen stimulation of CARs bearing the 4-1BB co-stimulation domain results in marked Th1 polarization and upregulation of the IL-21/IL-21R axis. FIG. 4A) Gene set enrichment analysis of an early polarizing T_{H}1 signature. Position of signature genes depicted in a rank fold-change list of the DE genes between bulk RNA-seq BBζ and 28ζ profiles 24 hours post-CAR activation with Nalm6 cells. FIG. 4B) Heat map of known T_{H}1 helper cell polarizing genes in CD4⁺ T cells from three donors in 28ζ vs. BBζ CARs 24 hours post-Nalm6 stimulation. FIG. 4C) IL-4 soluble cytokine detected in the supernatants of CD19-CAR T cells after 24 hours of Nalm6 stimulation and in FIG. 4D) EGFR-CAR T cells stimulated with U87 cells measured by luminex. N=3 normal donors. Mean and SEM plotted. p-values were determined using a paired student t-test between BBζ and 28ζ. *p<0.05

FIGS. 5A-5F. Antigen specific activation of 4-1BB CAR T cells induces a distanced program with additional genes networks than 4-1BB-ligand-mediated triggering of 4-1BB. Single cell expression profiles of CAR T cells after 24 hours of stimulation with Nalm6 cells were normalized and aligned across two donors. FIG. 5A) tSNE plot of single cell expression profiles (dots) shown by CAR T cell construct. FIG. 5B) Results of latent Dirichlet allocation on T cells with 16 topics and a tolerance parameter of 0.1 (Methods). For each topic shown there is a bar plot of top scoring genes (y axis), ranked by a uniqueness score. The genes in topic 11 which were also found as DE and upregulated (adj p<0.05) in BBζ vs 28ζ CAR T cells at any time point from bulk RNAseq data are shown. Each cell (dot) of the tSNE is shown by the weight of the topic. FIG. 5C) Gene regulators discovered using network analysis for each topic studied plotted by significance (−log(pval)). FIG. 5D) T cell activation expression level and FIG. 5E) degree of CD4 and CD8A expression in cells plotted in the tSNE as in FIG. 5A. FIG. 5F) Network of predicted transcription factors identified and the genes they regulate in the 4-1BB program (topic 11). FIG. 5G) Heatmap of endogenous 4-1BB stimulation relative to the 4-1BB signature defined as genes induced by BBζ in both bulk and scRNA-seq data. Untransduced T cells were stimulated for 24 hours with irradiated K562 expressing αCD3 with or without 4-1BBL to activate endogenous 4-1BB. N=3 normal donors.

FIGS. 6A-6D. Generation, quality and batch correction of CAR T cell profiles. FIG. 6A) Representative transduction efficiency of CAR constructs determined by mCherry expression and CD3 surface expression on day 13. FIG. 6B) Number of individual UMIs and the percent mitochondrial genes sequenced per sample loaded on the 10×. FIG. 6C) tSNE of scRNA-seq T cell profiles shown by donor pre and post CCA alignment (batch correction). FIG. 6D) Principal component analysis of bulk (donor 1-3) and summed single cell data (donor 4 and 5) after LIMMA correction for donor/sequencing method batch effects. Samples (data points) shown by donor and condition (top panel) or CD4 vs. CD8 (bottom panel).

FIGS. 7A-7I. Tonic Signaling signature in EGFR and CD19 CAR T cells. EGFR CARs were sorted on CD8⁺mCherry⁺ after 7 days of bead expansion and 7 days of rest. After sorting, RNA was isolated followed by reverse transcription to cDNA. Digital droplet PCR of genes upregulated (FIGS. 7A to 7E) and downregulated (FIGS. 7F and 7G) in our signature for tonic signaling from CD3ζ was performed. N=4 normal donors, mean and SEM plotted. Significance was determined with a paired-ratio student t-test comparing BBζ and ζ to Δζ and correcting for two comparisons with Holm-Bonferroni method adjustment. Genes are expressed relative to internal reference gene TBP. *adj-p<0.05**adj-p<0.01. FIG. 7H) tSNE of scRNA-seq T cell profiles from no stimulation conditions shown by donor after CCA batch correction. FIG. 7I) Number of individual UMIs and the percent mitochondrial genes sequenced per cluster from the unstimulated samples described in FIG. 2D.

FIGS. 8A-8B. DE genes from bulk RNA-seq between BBζ and 28ζ CAR T cells. FIG. 8A) Volcano plot of log fold-change genes expression on the x axis and −log 10(p value) on the y axis between CD19 BBζ and 28ζ CAR T cells at 0 and 4 hours post-Nalm6 activation in CD4⁺ and CD8⁺ cells. Genes with FDR<0.05 are plotted shown. Positive x-axis is up in BBζ vs. 28ζ. FIG. 8B) Classification of types of significantly differentially expressed genes at 24 hours after Nalm6 stimulation detected by bulk RNA-seq with an FDR<0.1 between BBζ and 28ζ CARs using GO annotation.

FIG. 9. BBζ CARs have increase fatty acid metabolism before activation. GSEA of hallmark fatty acid metabolism genes in rank fold-change list of DE genes between bulk RNA-seq profiles of CD19 BBζ and 28ζ CAR T cells at 0 hours and 4 hours post CAR activation with irradiated Nalm6 cells.

FIGS. 10A-10B. Bulk RNAseq profiles indicate cytokine and cytokine receptor differences in BBζ and 28ζ CAR T cells. Normalized gene expression in CD4⁺ and CD8⁺ in BBζ and 28ζ CD19 CAR T cells activated with irradiated Nalm6 of FIG. 10A) IL23R-IL-23 receptor, FIG. 10B) IL12RB2-IL-12 receptor and FIG. 10C) ENPP2-autotaxin. N=3 normal donors. Mean and SEM plotted with points showing individual donor values. Adj-p values were calculated by DEseq2 using Holm-Bonferroni correction. FIG. 10D) Bulk gene expression of PDCD1 (encoding the PD1 protein) in CD8⁺ CD19 CAR T cells with Nalm6 stimulation over time. Dots represent the individual donor samples. Mean and SEM plotted, *adj-p<0.05. FIG. 10E) GSEA of early polarizing T_{H}1 signature genes scored across the rank fold-change list of DE genes between BBζ (+) and 28ζ CAR T cells at the 0 hour time point. FIG. 10F) IL-5 measured in the supernatants of bulk CD4⁺/CD8⁺ EGFR CAR T cells stimulated for 24 hours with irradiated U87. N=3 normal donors, mean and SEM plotted. *p<0.05.

FIG. 11. Topics analysis of Nalm6 stimulated CAR T cells. Topics discovered by LDA (setting K parameter to 16) of single cell data from CD19-CAR BBζ, ζ, and 28ζ T cells 24 hours after Nalm6 stimulation. The tSNEs are shown by the weight of the given topic in each cell.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of, “consists” and “consists of”, as well as the terms “consisting essentially of, “consists essentially” and “consists essentially of”. It is noted that in this disclosure and particularly in the claims and/or paragraphs, terms such as “comprises”, “comprised”, “comprising” and the like can have the meaning attributed to it in U. S. Patent law; e.g., they can mean “includes”, “included”, “including”, and the like; and that terms such as “consisting essentially of” and “consists essentially of” have the meaning ascribed to them in U. S. Patent law, e.g., they allow for elements not explicitly recited, but exclude elements that are found in the prior art or that affect a basic or novel characteristic of the invention.

All publications, published patent documents, and patent applications cited herein are hereby incorporated by reference to the same extent as though each individual publication, published patent document, or patent application was specifically and individually indicated as being incorporated by reference.

### Overview

Described herein are embodiments of a cell atlas and immediate, tangible, and transformative benefits. Embodiments herein can provide a reference map for comparing related cells, identifying new cell types, helping interpret genetic variants, and identifying what distinguishes pathological cells from healthy ones. Knowing which genes are expressed in each tissue further helps to design better and safer therapeutics such as engineered CAR-Ts and address toxicity in drug development. Embodiments herein can define sets of markers and signatures, facilitating the development of relevant reagents (e.g., antibodies, probes) for molecular pathology, targeted cell sorting, and diverse additional assays. Embodiments described herein can provide a direct view of human biology in vivo because it is derived directly from human tissue, removing the distorting aspects of cell culture and allowing us to develop better models for basic biology and drug discovery. Embodiments described herein can also allow the effective deconvolution of a massive body of legacy data from individual studies to catalogs such as TCGA—to resolve the true content of current profiles, greatly enhancing their impact. Embodiments described herein can help identify the regulatory code that controls cell differentiation, maintains cell state, and underlies cell-cell interactions, all key targets for fundamental understanding and therapeutic intervention. Embodiments described herein can generate “hardened,” scaled, and broadly accepted methods for sample preparation, lab protocols, informatics infrastructure, and data analysis.

RNA sequencing of first-generation and second-generation human CAR T cells with 4-1BB (BBζ) or CD28 (28ζ) costimulatory domains at rest and following stimulation through their CAR or endogenous TCR was performed, mimicking encounter with tumor in patients. Described and demonstrated herein are variations in cytokine profiles, cytokine receptors, and metabolic pathways using differential gene expression analysis among CARs bearing CD3ζ activator vs. CARs bearing CD3ζ and a costimulatory domain of 1-4BB (“BBζ”) or CD28 (“28ζ”) intracellular signaling domains. A transcriptional signature present in resting CAR-modified T cells is also provided, indicating ligand-independent transcriptional activity of CARs. These transcriptional profiles can define antigen-dependent and antigen-independent CAR signaling pathways that ultimately determine CAR T cell fate.

Described herein are T cells that can be engineered to express a chimeric antigen receptor (CAR) and one or more costimulatory domains. In some aspects, the T cells described herein can have a specific gene signature or program. In some aspects the signature is that of a non-stimulated CAR T cells bearing CD3ζ and a costimulatory domain of 1-4BB (“BBζ”) or CD28 (“28ζ”) intracellular signaling domains. Other specific signatures are described herein. Also described herein are methods of generating such CAR T cells and methods of identifying and isolating candidate cells that have a desired gene signature or program. Also described herein are methods of administering isolated candidate cells to a subject in need thereof as and adoptive cell therapy.

### CAR T Cells

Described herein are T cells that can be engineered to express a chimeric antigen receptor (CAR) and one or more costimulatory domains. In some aspects, the T cells described herein can have a specific gene signature or biological program. In some aspects the signature is that of a non-stimulated CAR T cells bearing CD3ζ and a costimulatory domain of 1-4BB (“BBζ”) or CD28 (“28ζ”) intracellular signaling domains. Other specific signatures are described herein.

In some aspects, the T cell can be a metabolically enhanced T cell. In certain embodiments, a metabolically enhanced T cell can have a chimeric intracellular signaling molecule comprising an intracellular domain of a co-stimulatory molecule, and substantially lacks an extracellular ligand-binding domain. The metabolically enhanced T cell expresses the chimeric intracellular signaling molecule. In certain embodiments, expression of the chimeric intracellular signaling molecule metabolically enhances the T cell. In some embodiments, expression of the chimeric intracellular signaling molecule improves cytotoxicity and resistance to immunosuppression when in a tumor microenvironment.

### Signature Genes and Biological Programs

As used herein, a signature may encompass any gene or genes, or protein or proteins, whose expression profile or whose occurrence is associated with a specific cell type, subtype, or cell state of a specific cell type or subtype within a population of cells. Increased or decreased expression or activity or prevalence may be compared between different cells in order to characterize or identify for instance specific cell (sub)populations. A gene signature, as used herein, may thus refer to any set of up- and down-regulated genes between different cells or cell (sub)populations derived from a gene-expression profile. For example, a gene signature may comprise a list of genes differentially expressed in a distinction of interest. It is to be understood that also when referring to proteins (e.g. differentially expressed proteins), such may fall within the definition of “gene” signature.

The signatures as defined herein (being it a gene signature, protein signature or other genetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of immune cells that are linked to particular pathological condition (e.g. cancer), or linked to a particular outcome or progression of the disease, or linked to a particular response to treatment of the disease.

The signature according to certain embodiments of the present invention may comprise or consist of one or more genes and/or proteins, such as for instance 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of two or more genes and/or proteins, such as for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of three or more genes and/or proteins, such as for instance 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of four or more genes and/or proteins, such as for instance 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of five or more genes and/or proteins, such as for instance 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of six or more genes and/or proteins, such as for instance 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of seven or more genes and/or proteins, such as for instance 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of eight or more genes and/or proteins, such as for instance 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of nine or more genes and/or proteins, such as for instance 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more. In certain embodiments, the signature may comprise or consist of ten or more genes and/or proteins, such as for instance 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more.

In certain example embodiments, a CAR T cell can have a gene signature selected from:
a) a CD3ζ CAR T gene signature,
b) a costimulatory molecule gene signature,
c) a T_{H}1 response gene signature,
d) a T_{H}2 response gene signature,
e) a T cell activation gene signature,
f) any combination thereof.

A CD3ζ CAR T gene signature is a gene signature unique to CAR T cells expressing a CD3 ζ molecule. In some embodiments, the CD3ζ CAR T gene signature is present in CAR T cells that have not been stimulated (e.g. via their TCR and/or CAR). In some embodiments, the CD3ζ CAR T gene signature is present in CAR T cells that have been stimulated (e.g. via their TCR and/or CAR). In certain example embodiments, the CD3ζ CAR T gene signature comprises one or more signature genes selected from the group consisting of: ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof. In certain example embodiments, one or more signature genes in the CD3ζ CAR T gene signature are up-regulated, down-regulated, or both. In certain example embodiments, the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises ZP3 or GGT1. In certain example embodiments, the CD3ζ CAR T gene signature comprises CCL3, CCL4, GZMB, XCL1, ZBED2, IFNG, or any combination thereof.

A costimulatory molecule gene signature is a gene signature specific to a costimulatory molecule that can be present in a CAR T. Such costimulatory molecules can be CD28 zeta or BB1 zeta. Such specific costimulatory molecule gene signatures can also be referred to as, for example, a CD28 zeta gene signature or a BB1 zeta gene signature. In some aspects the costimulatory molecule signature can be unique to CD28 zeta (i.e. isa. CD28 zeta gene signature) or BB1 zeta (i.e. is a BB-1 gene signature). In certain example embodiments, the costimulatory molecule gene signature comprises one or more signature genes of Table 7, Table 8, or any combination thereof. In certain example embodiments, one or more signature genes in the costimulatory molecule gene signature are up-regulated, down-regulated, or both. In certain example embodiments, the costimulatory molecule gene signature comprises a gene signature selected from the group consisting of:

(a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6, ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
(b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
(c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, and any combination thereof;
(d) JUN, GPA33, KRT1, EGR1, CIITA, UBD, KLHL23, SCD, HLA-DOA, ALPK, CXCL10, and any combination thereof;
(e) JUN, EGR1, CIITA, GPA33, KRT1, and any combination thereof;
(f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
(g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
(h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
(i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
(j) CXCL10, JUNB, NTRK2, MSC, VNN2 and any combination thereof;
(k) JUNB, CXCL10, ENOX1, ENPP2, DDIT4, NTRK2, GCSAM, IL5, and any combination thereof;
(l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
(m) ENPP2, GJB2, C4orf26, MX1, NTRK2, JUNB, TNFRSF8, DGAT2, ELL2, IL4I1, ITPR1, HLA-DRB6, and any combination thereof, and
(n) CIITA, CD74, HLA-DMB, HLA-DPB1, HLA-DQA2, HLA-DRB1, HLA-DRB5, HLA-DOA, HLA-DRA, HLA-DRB6, and any combination thereof.

In certain example embodiments, the CAR T cell is CD4+. In certain example embodiments, the gene signature is any one of gene signatures (a)-(i).

In certain example embodiments, the CAR T cell is CD8+. In certain example embodiments, the gene signature is any one of gene signatures (a), (b), (c), (j), (k), (l), or (m).

In certain example embodiments, the CAR T cell is unstimulated. In certain example embodiments, the gene signature is any one of gene signatures (a), (d), (e), or (j).

In certain example embodiments, the CAR T cell is stimulated. In certain example embodiments, the gene signature is any one of gene signatures (b), (c), (f), (g), (h), (i), (k), (l), or (m).

In certain example embodiments, the CAR T cell expresses a CD28ζ co-stimulatory molecule. In certain example embodiments, one or more genes in any one of gene signatures (a)-(i) is up-regulated, down-regulated, or both as compared to a CAR T cell expressing a BBζ co-stimulatory molecule. In certain example embodiments, LGMN, PDCD1, GPA33, KRT1, VNN2, C17orf-PLSCR3, and any combination thereof is up-regulated in the CART cell as compared to a CAR T expressing a BBζ co-stimulatory molecule. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is down-regulated in the CART cell as compared to a CAR T expressing a BBζ co-stimulatory molecule.

In certain example embodiments, the CAR T cell expresses a BBζ co-stimulatory molecule. In certain example embodiments, one or more genes in any one of gene signatures (a)-(i) is up-regulated, down-regulated, or both as compared to a CAR T cell expressing a CD28ζ co-stimulatory molecule. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is up-regulated in the CAR T cell. In certain example embodiments, LGMN, PDCD1, GPA33, KRT1, VNN2, C17orf-PLSCR3, and any combination thereof is down-regulated in the CART cell.

A T_{H}1 response gene signature refers to a gene signature that is present in and/or identifies a cell that may promote a T_{H}1 response. In certain example embodiments, the T_{H}1 response gene signature comprises one or more signature genes selected from the group consisting of: ERG1, TBX21, RORC, IL12RB2, GLIL1, EPPN2, DMD, IFNG, and any combination thereof. In certain example embodiments, the CAR T cell expresses a BBζ co-stimulatory molecule. In certain example embodiments, the CAR T cell is CD4+.

A T_{H}2 response gene signature refers to a gene signature that is present in and/or identifies a cell that may promote a T_{H}2 response. In certain example embodiments, the T_{H}2 response gene signature comprises one or more signature genes selected from the group consisting of: IL4, IL5, IL2, and any combination thereof. In certain example embodiments, the CAR T cell expresses a CD28ζ co-stimulatory molecule. In certain example embodiments, the CAR T cell is CD4+.

In certain example embodiments, the T cell activation gene signature comprises one or more genes selected from Table 3, Table 4, or a combination thereof.

In certain example embodiments, the T cell activation gene signature comprises one or more genes selected from the group consisting of: IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof. In certain example embodiments, the stimulated CAR T cell was generated by stimulating the CAR T cell through a T cell receptor of the CAR T cell.

In certain example embodiments, IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are upregulated as compared to a CAR T cell stimulated through a CAR of the CAR T cell.

As is discussed elsewhere herein, activation of CAR T cells via the TCR can result in activation of different genes, pathways, and/or functions in the CAR T cells as compared to activation via a CAR present in the CAR T cell. The T cell activation gene signature reflects described herein reflects these differences resulting from different activation mechanisms. In some aspects, the T cell activation gene signature can be a CAR activation gene signature. In some aspects, the T cell activation gene signature can be a TCR activation gene signature. In certain example embodiments, the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1

TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

In certain example embodiments, the CAR T is a stimulated CAR T cell, wherein the stimulated CAR T cell was generated by stimulating a chimeric antigen receptor of CAR T cell.

In certain example embodiments, measuring expression of a gene signature comprises bulk RNA sequencing, single cell RNA sequencing (scRNA-seq), or both.

In certain example embodiments, the method further comprises isolating an identified candidate CAR T cell or a population thereof to obtain an isolated candidate CAR T cell or population thereof and optionally expanding the isolated candidate CAR T cell or population thereof to obtain an expanded candidate CAR T cell or population thereof.

In certain example embodiments, the method further comprises administering the isolated candidate CAR T cell or population thereof or the expanded candidate CAR T cell or population thereof to a subject in need thereof. In certain example embodiments, the subject in need thereof has a cancer.

For example, a signature for use in the disclosed detection methods can include a combination of genes either Table 1 of US Pat. App. Pub. 2019/0255107, Table 5 of US Pat. App. Pub. 2019/0255107, Table 6 of US Pat. App. Pub. 2019/0255107, Table 7 of US Pat. App. Pub. 2019/0255107, Table 8 of US Pat. App. Pub. 2019/0255107, Table 9 of US Pat. App. Pub. 2019/0255107 Table 10 of US Pat. App. Pub. 2019/0255107, Table 12 of US Pat. App. Pub. 2019/0255107, Table 13 of US Pat. App. Pub. 2019/0255107, Table 1 herein, or Table 2 herein. It is to be understood that a signature according to the invention may for instance also include a combination of genes or proteins.

TABLE 1
Pairs of Target Genes

| | Bst2 | Btla | Ccl9 | Ccr4 | Cd40lg | Cxcr4 | Gpr65 |
|---|---|---|---|---|---|---|---|
| Bst2 | | • | • | • | • | • | • |
| Btla | • | | • | • | • | • | • |
| Ccl9 | • | • | | • | • | • | • |
| Ccr4 | • | • | • | | • | • | • |
| Cd40lg | • | • | • | • | | • | • |
| Cxcr4 | • | • | • | • | • | | • |
| Gpr65 | • | • | • | • | • | • | |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Il33 | Klrc2 | Klrd1 | Klre1 | Lif | Lpar3 | Olfm1 |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | | • | • | • | • | • | • |
| Klrc2 | • | | • | • | • | • | • |
| Klrd1 | • | • | • | • | • | • | |
| Klre1 | • | • | • | | • | • | • |
| Lif | • | • | • | • | | • | • |
| Lpar3 | • | • | • | • | • | | • |
| Olfm1 | • | • | • | • | • | • | |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Il13ra1 | Il6st | Inhba | Lamp2 | Lap3 | Ly75 | Nampt |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | | • | • | • | • | • | • |
| I16st | • | | • | • | • | • | • |
| Inhba | • | • | | • | • | • | • |
| Lamp2 | • | • | • | | • | • | • |
| Lap3 | • | • | • | • | | • | • |
| Ly75 | • | • | • | • | • | | • |
| Nampt | • | • | • | • | • | • | |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Ccl5 | Cd83 | Klrk1 | Sema7a | Serpinc1 | Ccr2 | Ifitm1 |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | | • | • | • | • | • | • |
| Cd83 | • | | • | • | • | • | • |
| Klrk1 | • | • | | • | • | • | • |
| Sema7a | • | • | • | | • | • | • |
| Serpinc1 | • | • | • | • | | • | • |
| Ccr2 | • | • | • | • | • | | • |
| Ifitm1 | • | • | • | • | • | • | |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Il12rb1 | Il1r1 | Sdc4 | Slamf7 | Tgfb3 | Adam9 | Cd68 |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | | • | • | • | • | • | • |
| Il1r1 | • | | • | • | • | • | • |
| Sdc4 | • | • | | • | • | • | • |
| Slamf7 | • | • | • | | • | • | • |
| Tgfb3 | • | • | • | • | | • | • |
| Adam9 | • | • | • | • | • | | • |
| Cd68 | • | • | • | • | • | • | |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Tigit | Ccr5 | Adam8 | Cd68 | Isg20 | Il10 | Il10ra |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | | • | • | • | • | • | • |
| Ccr5 | • | | • | • | • | • | • |
| Adam8 | • | • | | • | • | • | • |
| Cd68 | • | • | • | | • | • | • |
| Isg20 | • | • | • | • | | • | • |
| Il10 | • | • | • | • | • | | • |
| Il10ra | • | • | • | • | • | • | |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | | | | | | | Havcr2 |
| | Il21 | Il2rb | Abca1 | Alcam | Cysltr2 | Gcnt1 | (Tim-3) |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | | • | • | • | • | • | • |
| Il2rb | • | | • | • | • | • | • |
| Abca1 | • | • | | • | • | • | • |
| Alcam | • | • | • | | • | • | • |
| Cysltr2 | • | • | • | • | | • | • |
| Gcnt1 | • | • | • | • | • | | • |
| Havcr2 (Tim-3) | • | • | • | • | • | • | |
| Gabarapl1 | • | • | • | • | • | • | • |
| Il2ra | • | • | • | • | • | • | • |
| Spp1 | • | • | • | • | • | • | • |
| Cxcl10 | • | • | • | • | • | • | • |
| Ifitm 3 | • | • | • | • | • | • | • |
| Il1r2 | • | • | • | • | • | • | • |
| Lag3 | • | • | • | • | • | • | • |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | Gabarapl1 | Il2ra | Spp1 | Cxcl10 | Ifitm3 | Il1r2 | Lag3 |
| Bst2 | • | • | • | • | • | • | • |
| Btla | • | • | • | • | • | • | • |
| Ccl9 | • | • | • | • | • | • | • |
| Ccr4 | • | • | • | • | • | • | • |
| Cd40lg | • | • | • | • | • | • | • |
| Cxcr4 | • | • | • | • | • | • | • |
| Gpr65 | • | • | • | • | • | • | • |
| Il33 | • | • | • | • | • | • | • |
| Klrc2 | • | • | • | • | • | • | • |
| Klrd1 | • | • | • | • | • | • | • |
| Klre1 | • | • | • | • | • | • | • |
| Lif | • | • | • | • | • | • | • |
| Lpar3 | • | • | • | • | • | • | • |
| Olfm1 | • | • | • | • | • | • | • |
| Pdpn | • | • | • | • | • | • | • |
| Ptpn3 | • | • | • | • | • | • | • |
| Sdc1 | • | • | • | • | • | • | • |
| Timp2 | • | • | • | • | • | • | • |
| Tnfsf9 (4-1BB) | • | • | • | • | • | • | • |
| Vldlr | • | • | • | • | • | • | • |
| Entpd1 | • | • | • | • | • | • | • |
| Il13ra1 | • | • | • | • | • | • | • |
| I16st | • | • | • | • | • | • | • |
| Inhba | • | • | • | • | • | • | • |
| Lamp2 | • | • | • | • | • | • | • |
| Lap3 | • | • | • | • | • | • | • |
| Ly75 | • | • | • | • | • | • | • |
| Nampt | • | • | • | • | • | • | • |
| Ccl5 | • | • | • | • | • | • | • |
| Cd83 | • | • | • | • | • | • | • |
| Klrk1 | • | • | • | • | • | • | • |
| Sema7a | • | • | • | • | • | • | • |
| Serpinc1 | • | • | • | • | • | • | • |
| Ccr2 | • | • | • | • | • | • | • |
| Ifitm1 | • | • | • | • | • | • | • |
| Il12rb1 | • | • | • | • | • | • | • |
| Il1r1 | • | • | • | • | • | • | • |
| Sdc4 | • | • | • | • | • | • | • |
| Slamf7 | • | • | • | • | • | • | • |
| Tgfb3 | • | • | • | • | • | • | • |
| Adam9 | • | • | • | • | • | • | • |
| Cd68 | • | • | • | • | • | • | • |
| Tigit | • | • | • | • | • | • | • |
| Ccr5 | • | • | • | • | • | • | • |
| Adam8 | • | • | • | • | • | • | • |
| Cd68 | • | • | • | • | • | • | • |
| Isg20 | • | • | • | • | • | • | • |
| Il10 | • | • | • | • | • | • | • |
| Il10ra | • | • | • | • | • | • | • |
| Il21 | • | • | • | • | • | • | • |
| Il2rb | • | • | • | • | • | • | • |
| Abca1 | • | • | • | • | • | • | • |
| Alcam | • | • | • | • | • | • | • |
| Cysltr2 | • | • | • | • | • | • | • |
| Gcnt1 | • | • | • | • | • | • | • |
| Havcr2 (Tim-3) | • | • | • | • | • | • | • |
| Gabarapl1 | | • | • | • | • | • | • |
| Il2ra | • | | • | • | • | • | • |
| Spp1 | • | • | | • | • | • | • |
| Cxcl10 | • | • | • | | • | • | • |
| Ifitm 3 | • | • | • | • | | • | • |
| Il1r2 | • | • | • | • | • | | • |
| Lag3 | • | • | • | • | • | • | |
| Pglyrp1 | • | • | • | • | • | • | • |
| Lilrb4 | • | • | • | • | • | • | • |
| Klrc1 | • | • | • | • | • | • | • |
| Procr | • | • | • | • | • | • | • |
| | | | | Pglyrp1 | Lilrb4 | Klrc1 | Procr |

| | | | | |
|---|---|---|---|---|
| Bst2 | • | • | • | • |
| Btla | • | • | • | • |
| Ccl9 | • | • | • | • |
| Ccr4 | • | • | • | • |
| Cd40lg | • | • | • | • |
| Cxcr4 | • | • | • | • |
| Gpr65 | • | • | • | • |
| Il33 | • | • | • | • |
| Klrc2 | • | • | • | • |
| Klrd1 | • | • | • | • |
| Klre1 | • | • | • | • |
| Lif | • | • | • | • |
| Lpar3 | • | • | • | • |
| Olfm1 | • | • | • | • |
| Pdpn | • | • | • | • |
| Ptpn3 | • | • | • | • |
| Sdc1 | • | • | • | • |
| Timp2 | • | • | • | • |
| Tnfsf9 (4-1BB) | • | • | • | • |
| Vldlr | • | • | • | • |
| Entpd1 | • | • | • | • |
| Il13ra1 | • | • | • | • |
| I16st | • | • | • | • |
| Inhba | • | • | • | • |
| Lamp2 | • | • | • | • |
| Lap3 | • | • | • | • |
| Ly75 | • | • | • | • |
| Nampt | • | • | • | • |
| Ccl5 | • | • | • | • |
| Cd83 | • | • | • | • |
| Klrk1 | • | • | • | • |
| Sema7a | • | • | • | • |
| Serpinc1 | • | • | • | • |
| Ccr2 | • | • | • | • |
| Ifitm1 | • | • | • | • |
| Il12rb1 | • | • | • | • |
| Il1r1 | • | • | • | • |
| Sdc4 | • | • | • | • |
| Slamf7 | • | • | • | • |
| Tgfb3 | • | • | • | • |
| Adam9 | • | • | • | • |
| Cd68 | • | • | • | • |
| Tigit | • | • | • | • |
| Ccr5 | • | • | • | • |
| Adam8 | • | • | • | • |
| Cd68 | • | • | • | • |
| Isg20 | • | • | • | • |
| Il10 | • | • | • | • |
| Il10ra | • | • | • | • |
| Il21 | • | • | • | • |
| Il2rb | • | • | • | • |
| Abca1 | • | • | • | • |
| Alcam | • | • | • | • |
| Cysltr2 | • | • | • | • |
| Gcnt1 | • | • | • | • |
| Havcr2 (Tim-3) | • | • | • | • |
| Gabarapl1 | • | • | • | • |
| Il2ra | • | • | • | • |
| Spp1 | • | • | • | • |
| Cxcl10 | • | • | • | • |
| Ifitm 3 | • | • | • | • |
| Il1r2 | • | • | • | • |
| Lag3 | • | • | • | • |
| Pglyrp1 | | • | • | • |
| Lilrb4 | • | | • | • |
| Klrc1 | • | • | | • |
| Procr | • | • | • | |

TABLE 2 (A-D)
Table 2A. IL-27-signature of up-regulated mouse genes expressed in several different
dysfunctional or tolerant T cell states.

| | | | | | | | |
|---|---|---|---|---|---|---|---|
| 1700012B09Rik | Cdh17 | Ets1 | Havcr2 | Klrc2 | Nfia | Rab31 | Sqrdl |
| AA467197 | Cdk6 | Etv6 | Hhat | Klrc2 | Nfil3 | Ramp3 | Srgap3 |
| Abca1 | Adkn2d | F2rl1 | Hhex | Klrd1 | Nkg7 | Rbp1 | Stat1 |
| Abcb9 | Cds2 | Fam129b | Hif1a | Klre1 | Oas2 | Rfk | Stat3 |
| Acadl | Cebpd | Fam20a | Hlx | Klrk1 | Ociad2 | Rgs1 | Stom |
| Adam19 | Cela1 | Rbxw7 | Hopx | Ksr1 | Oit3 | Rhoc | Styk1 |
| Adam8 | Cercam | Ffar2 | Hpse | Lag3 | Olfm1 | Rhoq | Syt11 |
| Adam9 | Chac1 | Fgl2 | Id2 | Lama5 | Ormdl3 | Ripk3 | Tbx21 |
| Agpat3 | Chit1 | Fhit | Ier3 | Lamp2 | Osr2 | Rnf125 | Tcp11l2 |
| Ahnak | Chm | Filipl | Ifih1 | Lat2 | Ovol2 | Rnh1 | Tgfb3 |
| Ahr | Chst11 | Flot1 | Ifitm1 | Lgals3 | Padi2 | Rorc | Tigit |
| Ahr | Chst2 | Fndc3a | Ifitm3 | Lgals3bp | Parp14 | Runx2 | Timp1 |
| Ak1 | Clip3 | Frmd4b | Igf2pb2 | Lilrb4 | Pdpn | S100a4 | Tmcc3 |
| Akrlb8 | Clybl | Gabarapl1 | Il10 | Litaf | Pfkp | S100a6 | Tnfrsf8 |
| Akrlb8 | Cni2 | Galc | Il10ra | Lpar3 | Pglyrp1 | Sccpdh | Tnfsf9 |
| Akt2 | Copz2 | Gatm | Il12rb1 | lpxn | Phactr2 | Sdc1 | Tor2a |
| Alcam | Creb312 | Gbe1 | Il13ra1 | Lrrk1 | Pik3ap1 | Sdc4 | Tpbg |
| Aldoc | Ctla2a | Gbp3 | Il1rl | Ltbp3 | Piwil2 | Sdcbp2 | Tpd52 |
| Anxa2 | Cxcl10 | Gbp3 | Il1r2 | Ly75 | Pkp2 | Sec24d | Trin3 |
| Anxa3 | Cysltr1 | Gpb6 | Il21 | Ly75 | Plac8 | Selenbp1 | Tspan4 |
| Aplp1 | Cysltr2 | Gcnt1 | Il2ra | Maf | Plekhf1 | Seim | Tspan5 |
| Aqp9 | Dapk2 | Gem | Il12rb | Map3k5 | Plekho2 | Selp | Ttc39b |
| Arfgap3 | Dclk1 | Gemin8 | Il33 | Med12l | P1ekho2 | Sema7a | Ttc39c |
| Arhgap18 | Ddr1 | Gfra1 | Il6st | Mettl7a1 | Plod2 | Serpinb1a | Tubb6 |
| Arl5a | Dhx58 | Gimap7 | Impa2 | Mmp15 | Ppme1 | Serpinb6b | Tulp4 |
| Armcx3 | Dock9 | Gja1 | Inhba | Ms4a6d | Ppplr3b | Serpinb9 | Ubac2 |
| Asb2 | Dst | Glg1 | Irf1 | Ms4a6d | Pqlc3 | Serpinf1 | Upp1 |
| Atf6 | E330009J07Rik | Glrx | Irf4 | Mt1 | Prdm1 | Sigirr | Usp18 |
| Atp6v0d2 | Eaf2 | Gmfg | Irf8 | Mt1 | Prex1 | Skap2 | Usp18 |
| Auh | Ecm1 | Gmppa | Irf9 | Mt1 | Prf1 | Slamf7 | Vldlr |
| Bcl2115 | Egln3 | Gnb5 | Isg15 | Mt1 | Procr | Slc2a3 | Wdr54 |
| Bnip3 | Elmo2 | Gnpda2 | Isg20 | Mt1 | Prss2 | Scl2a3 | Wdr81 |
| C3 | Emilin2 | Golga7 | Jun | Mt1 | Prss2 | Slc39a14 | Zbp1 |
| Ccl5 | Emp1 | Gpm6b | Junb | Mt2 | Prss2 | Slc41a2 | Zeb2 |
| Ccl9 | Enpp2 | Gpr65 | Kctd | Mxd1 | Psmb9 | Slc4a11 | Zfp36 |
| Ccl9 | Entpd1 | Gpt2 | Klf10 | Mxi1 | Pstpip1 | Slc7a3 | |
| Ccl9 | Entpd1 | Gsn | Klhl24 | Nampt | Ptpnl | Sord | |
| Ccr2 | Epcam | Gsn | Klrc2 | Ndrg1 | Ptpn3 | Sox5 | |
| Ccr5 | Ern1 | Gsn | Klrc2 | Neb | Pygl | Spats2 | |
| Cd68 | Erol1 | Gzmb | Klrc2 | Nedd4 | Rab11fip5 | Spp1 | |
| Cd93 | Enfi1 | Gzmc | Klrc2 | Nek6 | Rab27a | sqrdl | |

Table 2B. IK-27-signature of down-regulated mouse gene expressed in several
different dysfunctional or tolerant T cell states

| | | | | | | | |
|---|---|---|---|---|---|---|---|
| Aalf | Cd40lg | Dph5 | Gucy1b3 | Lrig1 | Phb | Rrs1 | Taf1d |
| Adi1 | Cd83 | Dus4l | Hells | Marcksl1 | Phlda1 | Rtp4 | Timm9 |
| Agpat5 | Cd8a | Egr3 | Hist2h3c1 | Mettl1 | Pkp4 | Sema4b | Timp2 |
| Akr1c18 | Cdk5r1 | Eomes | Id3 | Mmachc | Pmepa1 | Sema4c | Tm4sf5 |
| Akr1c18 | Chd9 | Fam26f | Idi2 | Mpeg1 | Prkcdbp | Serpinb6b | Tmem97 |
| Akr1c18 | Cnksr3 | Fhit | Ifih1 | Mtap | Prmt1 | Serpinb9 | Tnfaip8 |
| Akr1c18 | Cnn3 | Ftsj3 | Ifitn3 | Myb | Prmt3 | Serpinc1 | Tnfsf11 |
| Atp2a3 | Cpd | Galnt6 | Ipcef1 | Ndufa4 | Pter | Sh3bp5 | Top1mt |
| Bst2 | Crtam | Gch1 | Irf6 | Ndufaf4 | Ptger4 | Shmt1 | Trat1 |
| Btla | Csel1 | Gemin4 | Irgm1 | Nhp2 | Pus7l | S1amf6 | Trip13 |
| Cacnala | Csf2 | Gfi1 | Isg20 | Noc4l | Rcl1 | S1amf9 | Trpm1 |
| Cadm1 | Cxcl13 | Gnaq | Kbtbd8 | Nolc1 | Rcsd1 | Slc19a1 | Tsr2 |
| Camkk2 | Cxcr4 | Gnl3 | Klf10 | Nop16 | Rfc4 | Shg7 | Ttc27 |
| Capn3 | D930015E06Rik | Gpatch4 | Kti12 | Nop2 | Rnmtl1 | Snhg7 | Umps |
| Ccdc86 | Dapl1 | Gpd1l | Lad1 | Nop56 | Rpp14 | Snhg7 | Utp20 |
| Ccl1 | Ddit4 | Gramd1b | Lap3 | Nr4a3 | Rpp40 | St6gal | Wdr77 |
| Ccr4 | Ddx18 | Grwd1 | Lgals3bp | Pde7a | Rragd | St8sia4 | Zbtb10 |
| Cd226 | Dennd5a | Gucy1a3 | Lif | Pde8a | Rrp15 | Stc2 | Zfp608 |

Table 2C. IL27-signature of up-regulated human genes expressed in several different
dysfumctional or tolerant T cell states.

| | | | | | | | |
|---|---|---|---|---|---|---|---|
| ABCA1 | CD93 | ETS1 | BAVCR2 | KLRC2 | NEDD4 | PYGL | SPATS2 |
| ABCB9 | CDH17 | ETV6 | HHAT | KLRC3 | NEK6 | RAB11FIL5 | SPP1 |
| ACADL | CDK6 | F2RL1 | HHEX | KLRC4 | NFIA | RAB27A | SQRDL |
| ADAM19 | CDKN2D | FAM129B | HIF1A | KLRC4-KLRK1 | NFIL3 | RAB31 | SGRAP3 |
| ADAM8 | CDS2 | FAM20A | HLX | KLRD1 | NKG7 | RAMP3 | STAT1 |
| ADAM9 | CEBPD | FBXW7 | HOPX | KLRK1 | OA52 | RBP1 | STAT3 |
| AGPAT3 | CELA1 | FFAR2 | HPSE | KSR1 | OCIAD2 | RFK | STOM |
| AHNAK | CERCAM | FGL2 | ID2 | LAG3 | OIT3 | RGS1 | STYK1 |
| AHR | CHAC1 | FHIT | IER3 | LAMA5 | OLFM1 | RHOQ | SYT11 |
| AK1 | CHIT1 | FILIP1 | IFIH1 | LAMP2 | ORMDL3 | RIPK3 | TBX21 |
| AKR1B10 | CHM | FLOT1 | IFITM1 | LAT2 | OSR2 | RNF125 | TCP11L2 |
| AKR1B15 | CHST11 | FNDC3A | IFITM1 | LGALS3 | OVOL2 | RNH1 | TGFB3 |
| AKT2 | CHST2 | FRMD4B | IGF2BP2 | LGALS3BP | PADI2 | RORC | TIGIT |
| ALCAM | CLIP3 | GABARAPL1 | IL10 | LITAF | PARP14 | RUNX2 | TIMP1 |
| ALDOC | CLYBL | GALC | IL10RA | LPAR3 | PDPN | S100A4 | TMCC3 |
| ANXA2 | CNIH2 | GATM | IL12RB1 | LPXN | RFKP | S100A6 | TNFRSF8 |
| ANXA3 | COPZ2 | GBE1 | IL13RA1 | LRRK1 | PGLYRP1 | SCCPDH | TNFSF9 |
| APLP1 | CREB3L2 | GBP4 | IL1R1 | LTBP3 | PHACTR2 | SDC1 | TOR2A |
| AQP9 | CXCL10 | GBP6 | IL1R2 | LY75 | P1K3AP1 | SDC4 | TPBG |
| ARFGAP3 | CYSLTR1 | GBP7 | IL21 | LY75-CD302 | PIWIL2 | SDCBP2 | TPD52 |
| ARHGAP18 | CYSLTR2 | GCNT1 | IL2RA | MAF | PKP2 | SEC24D | TRIB3 |
| ARL5A | DAPK2 | GEM | IL2RB | MAP3K5 | PLAC8 | SELEMNP1 | TSPAN4 |
| ARMCX3 | DCLK1 | GEMIN8 | IL33 | MED12L | PLEKHF1 | SELP | TSPAN5 |
| ASB2 | DDR1 | GFRA1 | IL6ST | METTL7A | PLEKHO2 | SEMA7A | TTC39B |
| ATF6 | DHX58 | GIMAP7 | IMPA2 | MIMP15 | PLOD2 | SERPINB1 | TTC39C |
| ATP6VOD | DOXK9 | GJA1 | INHBA | MS4A6A | PPME1 | SERPINB6 | TUBB6 |
| AUH | DST | GLG1 | IRF1 | MS4A6E | PPP1R3B | SERPINB9 | TULP4 |
| BCL2L15 | EAF2 | GLRX | IRF4 | MT1B | PQLC3 | SERPINF1 | UBAC2 |
| BNIP | ECM1 | GMFG | IRF8 | MT1E | PRDM1 | SIGIRR | UPPI |
| C11ORF97 | EGLN3 | GMPPA | IRF9 | MT1F | PREX1 | SKAP2 | USP18 |
| C15ORF48 | ELMO2 | GNB5 | ISG15 | MT1G | PRF1 | SLAMF7 | USP41 |
| C3 | EMILIN2 | GNPDA2 | ISG20 | MT1M | PROCR | SLC2A14 | VLDLR |
| CCL15 | EMP1 | GOLGA7 | JUN | MT1X | PRSS1 | SLC2A3 | WDR54 |
| CCL15-CCL14 | ENPP2 | GPM6B | JUNB | MT2A | PRSS2 | SLC39A14 | WDR81 |
| CCL23 | ENTPD1 | GPR65 | KCTD11 | MXD1 | PRSS3 | SLC41A2 | ZBP1 |
| CCL5 | EPCAM | GPT2 | KIAA1147 | MXL1 | PSMB9 | SLC4A11 | ZEB2 |
| CCR2 | ERN1 | GSN | KLF10 | NAMPT | PSTPIP1 | SLC7A3 | ZEPF36 |
| CCR2 | ERO1A | GZMB | KLHL24 | NDRG1 | PTPN1 | SORD | |
| CD68 | ERRFL1 | GZMB | KLRC1 | NEB | PTPN3 | SOX5 | |

Table 2D. IL-27-signature of down-regulated human genes expressed in several
different dysfunctional or tolerant T cell states.

| | | | | | | | |
|---|---|---|---|---|---|---|---|
| AATF | CD40LG | ERG3 | HIST2H3C | LRIG1 | PDE8A | RRS1 | TIMP2 |
| ADL1 | CD83 | EOMES | ID3 | MARCKSL1 | PHB | RTP4 | TM4SF5 |
| AGPAT5 | CD8A | FAM26F | ODO2 | METTL1 | PHLDA1 | SEMA4B | TMEM97 |
| AKR1C1 | CDK5R1 | IFIH1 | IFIH1 | MMACHC | PKP4 | SEMA4C | TNFA1P8 |
| AKR1C2 | CHD9 | FTSJ3 | IFITM1 | MPEG1 | PMEPA1 | SERPINB6 | TNFSF11 |
| AKR1C3 | CNN3 | GALNT6 | IPCEF1 | MRM3 | PRKCDBP | SERPINB9 | TOP1MT |
| AKR1C4 | CPD | GCH1 | IPCEF1 | MTAP | PRMT1 | SERPINC1 | TRAT1 |
| ATP2A3 | CRTAM | GEMIN4 | IRF6 | MYB | PRMT3 | SH3BP5 | TRIP13 |
| BST2 | CSE1L | GFI1 | IRGM | MDIFA4 | PTER | SHMT1 | TRPM1 |
| BTLA | CSF2 | GNAQ | ISG20 | NDUFAF4 | PTGER4 | SLAMF6 | TSR2 |
| CACNA1A | CXCL13 | GNL3 | KBTBD8 | NHP2 | PUS7L | FLAMF9 | TTC27 |
| CADM1 | CXCR4 | GPATCH4 | KIAA0922 | NOC4L | RCL1 | SLC19A1 | UMPS |
| CAMK2 | DAPL1 | GPD1L | KLF10 | NOLC1 | RCSD1 | SNORA17B | UTP20 |
| CAPN3 | DDIT4 | DRAMD1B | LTO12 | NOP16 | RFC4 | ST6GAL1 | WDR77 |
| CCDC86 | DDX18 | GRWD1 | LAD1 | NOP2 | RPP14 | ST8SIA4 | ZBTB10 |
| CCL1 | DENND5A | GUCY1A3 | LAP3 | NOP56 | RPP40 | STC2 | ZNF608 |
| CCR4 | DPH5 | GUCY1B3 | LGALS3BP | NR4A3 | RRAGD | TAF1D | |
| CD226 | DUS4L | HELLS | LIF | PDE7A | RRP15 | TIMM9 | |

It is to be understood that “differentially expressed” genes/proteins include genes/proteins which are up- or down-regulated as well as genes/proteins which are turned on or off. When referring to up- or down-regulation, in certain embodiments, such up- or downregulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins may be differentially expressed on a single cell level, or may be differentially expressed on a cell population level. Preferably, the differentially expressed genes/proteins as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein of the signature, such as for instance at least two, at least three, at least four, at least five, at least six, or all genes/proteins of the signature.

Signatures may be functionally validated as being uniquely associated with a particular immune phenotype. Induction or suppression of a particular signature may consequentially be associated with or causally drive a particular immune phenotype.

Various aspects and embodiments of the invention may involve analyzing gene signatures, protein signatures, and/or other genetic signatures based on single cell analyses (e.g. single cell RNA sequencing) or alternatively based on cell population analyses, as is defined herein elsewhere.

In further aspects, the invention relates to gene signatures, protein signatures, and/or other genetic signatures of particular immune cell subpopulations, as defined herein. The invention hereto also further relates to particular immune cell subpopulations, which may be identified based on the methods according to the invention as discussed herein; as well as methods to obtain such cell (sub)populations and screening methods to identify agents capable of inducing or suppressing particular immune cell (sub)populations.

The invention further relates to various uses of the gene signatures, protein signatures, and/or other genetic signatures as defined herein, as well as various uses of the immune cells or immune cell (sub)populations as defined herein. Particular advantageous uses include methods for identifying agents capable of inducing or suppressing particular immune cell (sub)populations based on the gene signatures, protein signatures, and/or other genetic signatures as defined herein. The invention further relates to agents capable of inducing or suppressing particular immune cell (sub)populations based on the gene signatures, protein signatures, and/or other genetic signatures as defined herein, as well as their use for modulating, such as inducing or repressing, a particular gene signature, protein signature, and/or other genetic signature. In related aspects, modulating, such as inducing or repressing, a particular gene signature, protein signature, and/or other genetic signature may modify overall immune cell composition, such as activated or dysfunctional immune cell composition, or distribution, or functionality.

As used herein the term “signature gene” means any gene or genes whose expression profile is associated with a specific cell type, subtype, or cell state of a specific cell type or subtype within a population of cells. The signature gene can be used to indicate the presence of a cell type, a subtype of the cell type, the state of the microenvironment of a population of cells, and/or the overall status of the entire cell population. Furthermore, the signature genes may be indicative of cells within a population of cells in vivo. Not being bound by a theory, the signature genes can be used to deconvolute the cells present in a tumor based on comparing them to data from bulk analysis of a tumor sample. The signature gene may indicate the presence of one particular cell type. In one embodiment, the signature genes may indicate that dysfunctional or activated tumor infiltrating T-cells are present. The presence of cell types within a tumor may indicate that the tumor will be resistant to a treatment. In one embodiment, the signature genes of the present invention are applied to bulk sequencing data from a tumor sample to transform the data into information relating to disease outcome and personalized treatments. In one embodiment, the novel signature genes are used to detect multiple cell states that occur in a subpopulation of tumor cells that are linked to resistance to targeted therapies and progressive tumor growth. In preferred embodiments, immune cell states of tumor infiltrating lymphocytes are detected.

In one embodiment, the signature genes are detected by immunofluorescence, mass cytometry (CyTOF), FACS, drop-seq, RNA-seq, single cell qPCR, MERFISH (multiplex (in situ) RNA FISH), microarray and/or by in situ hybridization. Other methods, including absorbance assays and colorimetric assays, are known in the art and may be used herein. In some aspects, measuring expression of signature genes comprises measuring protein expression levels. Protein expression levels may be measured, for example, by performing a Western blot, an ELISA or binding to an antibody array. In another aspect, measuring expression of said genes comprises measuring RNA expression levels. RNA expression levels may be measured by performing RT-PCR, Northern blot, an array hybridization, or RNA sequencing methods.

### Biological Programs

As used herein the term “biological program” can be used interchangeably with “expression program” or “transcriptional program” and may refer to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program. The biological program can be modeled as a distribution over expressed genes. One method to identify cell programs is non-negative matrix factorization (NMF) (see, e.g., Lee D D and Seung H S, Learning the parts of objects by non-negative matrix factorization, Nature. 1999 Oct. 21; 401(6755):788-91). Other approaches are topic models (Bielecki, Riesenfeld, Kowalczyk, et al., 2018 Skin inflammation driven by differentiation of quiescent tissue-resident ILCs into a spectrum of pathogenic effectors. bioRxiv 461228) and word embeddings. Identifying cell programs can recover cell states and bridge differences between cells. Single cell types may span a range of continuous cell states (see, e.g., Shekhar et al., Comprehensive Classification of Retinal Bipolar Neurons by Single-Cell Transcriptomics Cell. 2016 Aug. 25; 166(5):1308-1323.e30; and Bielecki, Riesenfeld, Kowalczyk, et al., 2018 Skin inflammation driven by differentiation of quiescent tissue-resident ILCs into a spectrum of pathogenic effectors. bioRxiv 461228).

To identify more nuanced changes in expression programs in response to e.g. stimulus, costimulatory molecule expression, etc., reliance on topic modeling using Latent Dirichlet Allocations (Blei et al., 2003), has recently been applied to scRNA-seq data (Bielecki et al., 2018; duVerle et al., 2016). Originally developed to discover key semantic topics reflected by the words used in a corpus of documents (Dumais et al., 1990), topic modeling can be used to explore gene programs (“topics”) in each cell (“document”) based on the distribution of genes (“words”) expressed in the cell. A gene can belong to multiple programs, and its relative relevance in the topic is reflected by a weight. A cell is then represented as a weighted mixture of topics, where the weights reflect the importance of the corresponding gene program in the cell. Additional details are provided elsewhere herein. See e.g. at least the working examples herein.

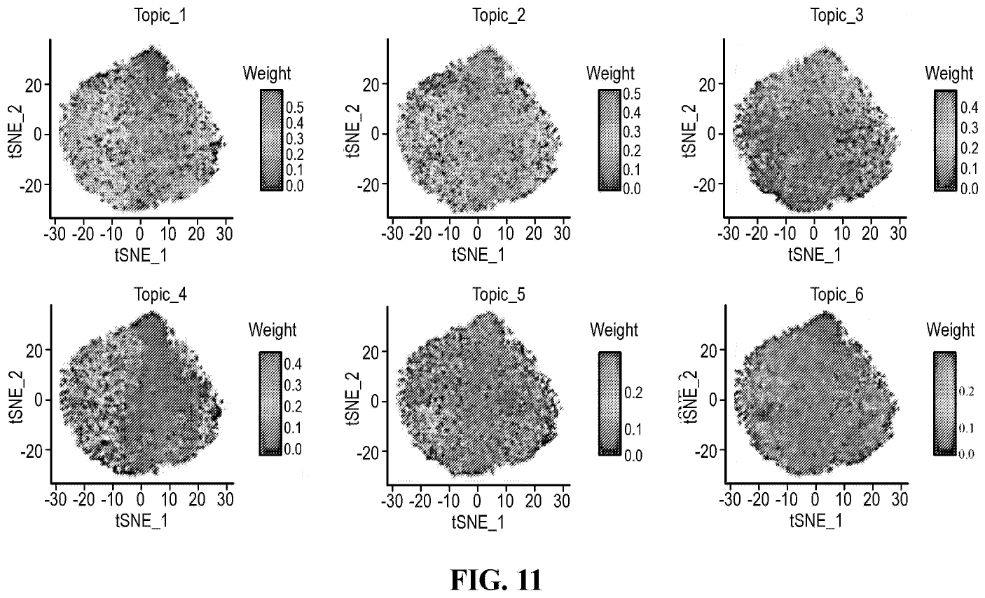

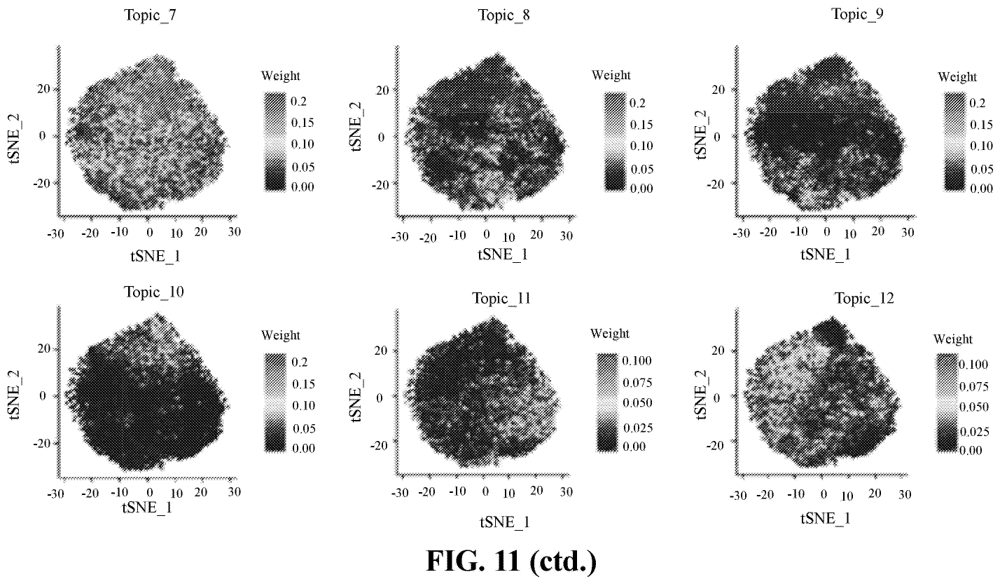

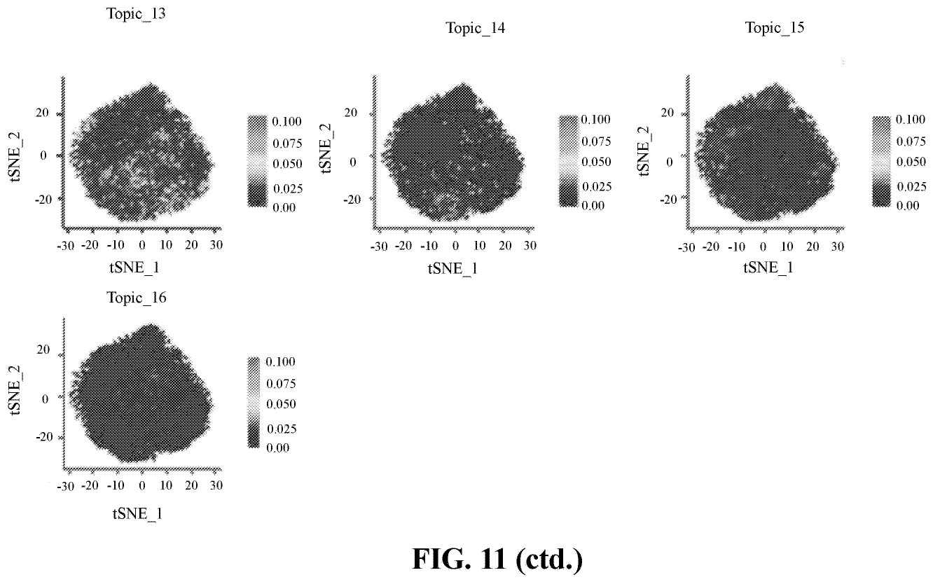

The biological programs described herein can include any of the signature genes described herein. In some embodiments, the biological program can include one or more of Topics 1-15 as described in the working examples herein (see also e.g. FIG. 11).

### Methods and Compositions for Cells Expressing a Chimeric Intracellular Signaling Molecule

In yet another aspect, the invention includes a metabolically enhanced T cell comprising a chimeric intracellular signaling molecule comprising an intracellular domain of a co-stimulatory molecule, and substantially lacks an extracellular ligand-binding domain. The metabolically enhanced T cell expresses the chimeric intracellular signaling molecule. In certain embodiments, expression of the chimeric intracellular signaling molecule metabolically enhances the T cell. In some embodiments, expression of the chimeric intracellular signaling molecule improves cytotoxicity and resistance to immunosuppression when in a tumor microenvironment.

The invention also includes a combination approach for adoptive cell therapy by arming the metabolically enhanced T cells with bispecific antibodies (BiAb). The presence of T regulatory cells (CD4+/CD25^{hi}/CD127^{lo}), granulocytic (CD14⁻/HLA-DR⁻/CD11b⁺/CD33⁺) and monocytic (CD14⁺/HLA-DR⁻/CD11b⁺/CD33⁺) myeloid derived suppressor cell (MDSC) populations modify the tumor microenvironment to sabotage the ability of incoming immune effector cells. In vitro models have shown that T cells armed with bispecific antibodies inhibit MDSC differentiation and attenuate T regulatory and MDSC suppressor activity (Thakur A, et al., J Transl Med., 11:35, 2013). Arming T cells with bispecific antibodies also induced the cells to secrete Th1 cytokines, kill target cells, and expand after tumor engagement to shift the tumor microenvironment to a Th1 environment (Grabert, R. C., et al., Clin. Canc. Res., 12:569-576, 2006) and vaccinate the patient with their own tumor antigens.

Based on observations of these modified T cells, a new modality of T cell therapy has been developed and is described herein using metabolically enhanced T cells alone or metabolically enhanced T cells that also encode a BiAb to improve treatment efficacy.

### Chimeric Intracellular Signaling Molecule

The present invention includes a chimeric intracellular signaling molecule within a T cell described herein. In one aspect, the invention includes a modified T cell comprising an isolated nucleic acid sequence encoding a chimeric intracellular signaling molecule, wherein the isolated nucleic acid sequence comprises a nucleic acid sequence of an intracellular domain of a co-stimulatory molecule and substantially lacks an extracellular ligand-binding domain, wherein the T cell expresses the chimeric intracellular signaling molecule.

In another aspect, the invention includes a modified T cell comprising a chimeric intracellular signaling molecule, wherein the chimeric intracellular signaling molecule comprises an intracellular domain of a co-stimulatory molecule and substantially lacks an extracellular ligand-binding domain.

In yet another aspect, the invention includes a population of cells comprising a nucleic acid encoding a chimeric intracellular signaling molecule comprising an intracellular domain and substantially lacks an extracellular ligand-binding domain, wherein the population of cells express the chimeric intracellular signaling molecule.

In one embodiment, the chimeric intracellular signaling molecule lacks any functional ligand-binding domain in the extracellular domain, such as lacking an antigen binding domain. In another embodiment, the chimeric intracellular signaling molecule includes an extracellular domain, but lacks the capacity to specifically bind to a ligand or molecule. In yet another embodiment, the chimeric intracellular signaling molecule substantially lacks an extracellular domain.

In another aspect, the invention includes a metabolically enhanced T cell comprising a chimeric intracellular signaling molecule comprising an intracellular domain of a co-stimulatory molecule and an extracellular domain comprising a non-antigen binding domain of an antibody, such as a single chain fragment comprising the non-antigen binding portion and lacking the variable region or a Fc portion of an antibody, i.e., IgD or IgA.

In yet another aspect, the invention includes a metabolically enhanced T cell comprising a chimeric intracellular signaling molecule comprising an intracellular domain of a co-stimulatory molecule, and substantially lacks an extracellular ligand-binding domain. The metabolically enhanced T cell expresses the chimeric intracellular signaling molecule. In certain embodiments, expression of the chimeric intracellular signaling molecule metabolically enhances the T cell. In some embodiments, expression of the chimeric intracellular signaling molecule improves cytotoxicity and resistance to immunosuppression when in a tumor microenvironment.

### Intracellular Domain

The intracellular domain or otherwise the cytoplasmic domain of the chimeric intracellular signaling molecule of the invention, is responsible for activation of the cell in which the chimeric intracellular signaling molecule is expressed. The term “intracellular domain” is thus meant to include any truncated portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines.

In one embodiment, the intracellular domain includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

In one embodiment, a cell comprising a chimeric intracellular signaling molecule is metabolically enhanced. In another embodiment, a cell comprising a chimeric intracellular signaling molecule has improved cytotoxicity and resistance to immunosuppression, such as when the cell is in a tumor microenvironment.

Examples of an intracellular domain for use in the invention include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability.

Examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In one embodiment, the intracellular domain of the chimeric intracellular signaling molecule includes any portion of a co-stimulatory molecule, such as at least one signaling domain from CD3, CD27, CD28, ICOS, 4-1BB, PD-1, T cell receptor (TCR), co-stimulatory molecules, any derivative or variant of these sequences, any synthetic sequence that has the same functional capability, and any combination thereof.

### Other Domains of the Chimeric Intracellular Signaling Molecule

The chimeric intracellular signaling molecule may include a detectable tag. As used herein, the term “protein tag” or “detectable tag” or “tag” generally means any oligo- or polypeptide that is connected to the chimeric intracellular signaling molecule to detect the chimeric intracellular signaling molecule. The tag may be attached to the intracellular domain, a hinge domain, a transmembrane domain or other domain of the chimeric intracellular signaling molecule. The tag may include up to 100 amino acids, between 10 to 50 amino acids, or between 5 to 25 amino acids. The tag may be removed or cleaved from the chimeric intracellular signaling molecule by a chemical agent or enzyme, such as protease, intein splicing, peptidase, etc. The tag may also be used for affinity purification of the chimeric intracellular signaling molecule. Examples of a tag includes, but is not limited to, chitin binding protein, maltose binding protein, thioredoxin-tag, fluorescent tag, glutathione-S-transferase (GST), poly(His) tag, V5-tag, Myc-tag, HA-tag, biotin or biotin-like molecules, streptavidin binding molecules, FLAG tag, or other tags known in the art.

The chimeric intracellular signaling molecule may include a spacer domain. As used herein, the term “spacer domain” generally means any oligo- or polypeptide that functions to link any domains, such as linking the transmembrane domain to, either the extracellular domain or, the cytoplasmic domain in the polypeptide chain. A spacer domain may be on one or both ends of the chimeric intracellular signaling molecule. A spacer domain may comprise up to 300 amino acids, preferably 10 to 100 amino acids and most preferably 25 to 50 amino acids.

In some embodiments, the chimeric intracellular signaling molecule further comprises a hinge and/or transmembrane domain. In one embodiment, the chimeric intracellular signaling molecule further comprises a hinge and/or transmembrane domain, such as a CD28 transmembrane domain and a CD8-alpha hinge domain. Examples of the hinge and/or transmembrane domain include, but are not limited to, a hinge and/or transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIR, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, NKG2C, and any combination thereof.

### Costimulatory Signaling Domains

The T cell can be engineered to include a costimulatory molecule or signaling domain. The costimulatory signaling molecule or domain can be integerated with or coupled to a CAR or chimeric signaling molecule described elsewhere herein. In certain embodiments, the one or more costimulatory signaling domains comprise a functional signaling domain of a protein selected, each independently, from the group consisting of: CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain preferred embodiments, the one or more costimulatory signaling domains comprise a functional signaling domain of a protein selected, each independently, from the group consisting of: 4-1BB, CD27, and CD28. In certain preferred embodiments, the costimulatory signaling domain comprises a functional signaling domain of CD28. In certain embodiments, the CAR comprises an anti-CD19 scFv, an intracellular domain of a CD3ζ chain, and a signaling domain of CD28. In certain preferred embodiments, the CD28 sequence is as set forth in Genbank identifier NM_006139 (sequence version 1, 2 or 3) starting with the amino acid sequence IEVMYPPPY and continuing all the way to the carboxy-terminus of the protein.

### Bispecific Antibody Armed T Cells

The invention also includes a combination approach for adoptive cell therapy by arming the T cells described herein, such as a candidate cell, with bispecific antibodies (BiAb). The presence of T regulatory cells (CD4+/CD25^{hi}/CD127^{lo}), granulocytic (CD14⁻/HLA-DR⁻/CD11b⁺/CD33⁺) and monocytic (CD14⁺/HLA-DR⁻/CD11b⁺/CD33⁺) myeloid derived suppressor cell (MDSC) populations modify the tumor microenvironment to sabotage the ability of incoming immune effector cells. In vitro models have shown that T cells armed with bispecific antibodies inhibit MDSC differentiation and attenuate T regulatory and MDSC suppressor activity (Thakur A, et al., J Transl Med., 11:35, 2013). Arming T cells with bispecific antibodies also induced the cells to secrete Th1 cytokines, kill target cells, and expand after tumor engagement to shift the tumor microenvironment to a Th1 environment (Grabert, R. C., et al., Clin. Canc. Res., 12:569-576, 2006) and vaccinate the patient with their own tumor antigens.

Based on observations of these modified T cells, a new modality of T cell therapy has been developed and is described herein using T cells described herein alone or T cells described herein that also encode a BiAb to improve treatment efficacy. The metabolically enhanced T cell described elsewhere herein may be armed with the bispecific antibody. In some embodiments, when a T cell described herein is armed with the bispecific antibody, the cell is contacted with bispecific antibody and the bispecific antibody specifically binds to an antigen on the surface of the cell through one antigen binding domain of the bispecific antibody. In other embodiments, the T cell can be armed with two or more bispecific antibodies and the T cell displays the two or more bispecific antibodies. In such an embodiment, the T cell specifically binds to at least two of the bispecific antibodies.

### Bispecific Antibodies

Described herein are bispecific antibodies that can be used to arm a T cell as elsewhere described herein. The bispecific antibody comprises two different binding specificities and thus binds to two different antigens. In such an embodiment, the bispecific antibody comprises a first antigen binding domain that binds to a first antigen and a second antigen binding domain that binds to a second antigen. The bispecific antibody may specifically bind to more than one epitope on the same target, such as a cell or receptor, or to more than one epitope on different targets. In another embodiment, the bispecific antibody comprises a bispecific antigen binding domain.

The present invention should not be construed to be limited to any particular bispecific antibody. Rather, any bispecific antibody is useful in the present invention. The bispecific antibody may be constructed from a synthetic antibody, a human antibody, a humanized antibody, a single chain variable fragment (scFv), a single domain antibody, an antigen binding fragment thereof, and any combination thereof. In one embodiment, the bispecific antibody is constructed by linking two different antibodies, or portions thereof, such as Fab, F(ab′)₂, Fab′, scFv, and sdAb from two different antibodies. Techniques for making human and humanized antibodies and antibody fragments, such as a scFv, are also described elsewhere herein. In another embodiment, the bispecific antibody comprises an antigen binding domain comprising a first and a second single chain variable fragment (scFv) molecule.

Techniques for engineering and expressing bispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science 229:81 (1985)); using leucine zippers to produce bispecific antibodies (see, e.g., Kostelny et al., J. Immunol. 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991). Engineered antibodies with three or more functional antigen binding sites, including “Octopus antibodies,” are also included herein (see, e.g. US 2006/0025576A1).

The present invention includes a bispecific antibody having an antigen binding domain that binds to a target cell. In one embodiment, the bispecific antibody has specificity for a target cell antigen. In another embodiment, the bispecific antibody comprises an antibody or fragment thereof that specifically binds to the target cell antigen. The target cell antigen may include the same target cell antigen that the T cell receptor binds to or may include a different target cell antigen. The target cell antigen may include any type of ligand found on the surface of a target cell including ligands on T cells (e.g. CD3, CD2, or other antigens expressed on T cell blasts). For example, the target cell antigen may be chosen because it recognizes a ligand that acts as a cell marker on a target cell that is associated with a particular disease state. Thus, examples of cell markers that may act as ligands for the antigen moiety domain in a bispecific antibody include those associated with viral, bacterial and parasitic infections, autoimmune disease and cancer cells. In one embodiment, the target cell antigen includes any tumor associated antigen (TAA), any viral antigen, or any fragment thereof.

In another embodiment, the bispecific antibody has specificity for at least one antigen on a T cell, such as the metabolically enhanced T cell as described elsewhere herein. The T cell antigen includes an antigen found on the surface of a T cell. The T cell antigen may include a co-stimulatory molecule as described elsewhere herein. In one embodiment, the T cell antigen is CD3, CD4, CD8, T cell receptor (TCR), or any fragment thereof. In this embodiment, the bispecific antibody comprises an antibody that specifically binds to the T cell antigen. Examples of the bispecific antibody may include anti-CD3, anti-CD4, anti-CD8, anti-TCR, anti-IgD Fc, anti-IgA Fc, any fragment thereof, and any combination thereof. The other target antigen of the bispecific antibody could also be a T cell antigen such as CD3, CD4, CD8, TCR, or any fragment thereof (e.g. anti-CD3×anti-CD3 construct). In another embodiment, the bispecific antibody is chemically heterconjugated to a polyclonal antibody specific for a tumor-associated antigen (TAA), and the T cell specifically binds the TAA polyclonal antibody.

Another embodiment of the invention includes the metabolically enhanced T cell and/or other T cell described herein wherein the first antigen binding domain binds to a target cell and a second antigen binding domain binds to an activated T cell.

The metabolically enhanced T cell and/or other T cell described elsewhere herein may be armed with the bispecific antibody. When a cell is armed with the bispecific antibody, the cell is contacted with bispecific antibody and the bispecific antibody specifically binds to an antigen on the surface of the cell through one antigen binding domain of the bispecific antibody. In another embodiment, the T cell is armed with two or more bispecific antibodies and the T cell displays the two or more bispecific antibodies. In such an embodiment, the T cell specifically binds to at least two of the bispecific antibodies.

Alternatively, the bispecific antibody may be expressed and secreted by the cell. When the cell expresses the bispecific antibody, a nucleic acid sequence encoding the bispecific antibody may be introduced into the cell. The nucleic acid sequence may be introduced by any method described elsewhere herein or other methods known in the art. In one embodiment, the cell is electroporated with a nucleic acid sequence encoding the bispecific antibody.

### Chimeric Antigen Receptor (CAR)

In some aspects, the T cells described herein, including metabolically enhanced T cells, are engineered to express a CAR. In some aspects, the T cell is generated by expressing a CAR therein. Thus, the present invention encompasses a CAR and a nucleic acid construct encoding a CAR, wherein the CAR includes an antigen binding domain, a transmembrane domain and an intracellular domain.

One or more domains or a fragment of a domain of the CAR may be human. In one embodiment, the present invention includes a fully human CAR. The nucleic acid sequences coding for the desired domains can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than as a cloned molecule.

In one aspect, the invention includes a metabolically enhanced, tumor specific T cell comprising a chimeric antigen receptor (CAR) and a bispecific antibody, wherein the CAR comprises an antigen binding domain, a transmembrane domain and an intracellular domain, and the bispecific antibody binds to a target on a tumor cell and the T cell, and wherein the T cell has improved cytotoxicity and resistance to immunosuppression at a solid tumor site. In one embodiment, the T cell comprises a nucleic acid sequence encoding the CAR and, optionally, a nucleic acid sequence encoding the bispecific antibody. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

### Antigen Binding Domain

In one embodiment, the CAR of the invention comprises an antigen binding domain that binds to an antigen on a target cell. Examples of cell surface markers that may act as an antigen that binds to the antigen binding domain of the CAR include those associated with viral, bacterial and parasitic infections, autoimmune disease, and cancer cells.

The choice of antigen binding domain depends upon the type and number of antigens that are present on the surface of a target cell. For example, the antigen binding domain may be chosen to recognize an antigen that acts as a cell surface marker on a target cell associated with a particular disease state.

In one embodiment, the antigen binding domain binds to a tumor antigen, such as an antigen that is specific for a tumor or cancer of interest. In one embodiment, the tumor antigen of the present invention comprises one or more antigenic cancer epitopes.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof.

In some instances, it is beneficial for the antigen binding domain to be derived from the same species in which the CAR will ultimately be used in. For example, for use in humans, it may be beneficial for the antigen binding domain of the CAR to comprise a human antibody, humanized antibody as described elsewhere herein, or a fragment thereof.

It is also beneficial that the antigen binding domain is operably linked to another domain of the CAR, such as the transmembrane domain or the intracellular domain, both described elsewhere herein, for expression in the cell. In one embodiment, a nucleic acid encoding the antigen binding domain is operably linked to a nucleic acid encoding a transmembrane domain and a nucleic acid encoding an intracellular domain.

In some embodiments, the antigen binding domain can be specific (i.e. specifically bind) to an antigen on a target cell. In some aspects, the target cell is a cancer cell. In some aspects the antigen binding domain can be specific for a cancer associated antigen. Cancer associated antigens are described elsewhere herein. A CAR described herein can comprise an antigen binding domain (e.g., antibody or antibody fragment, TCR or TCR fragment) that binds to a tumor-supporting antigen (e.g., a tumor-supporting antigen as described herein). Tumor supporting antigens are described in greater detail elsewhere herein.

### Transmembrane Domain

With respect to the transmembrane domain, the CAR can be designed to comprise a transmembrane domain that connects the antigen binding domain of the CAR to the intracellular domain. In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well, including the human Ig (immunoglobulin) hinge.

In one embodiment, the transmembrane domain may be synthetic, in which case it will comprise predominantly hydrophobic residues such as leucine and valine. Preferably, a triplet of phenylalanine, tryptophan and valine will be found at each end of a synthetic transmembrane domain.

### Intracellular Domain

The intracellular domain or otherwise the cytoplasmic domain of the CAR includes a similar or the same intracellular domain as the chimeric intracellular signaling molecule described elsewhere herein, and is responsible for activation of the cell in which the CAR is expressed.

In one embodiment, the intracellular domain of the CAR includes a domain responsible for signal activation and/or transduction.

Examples of an intracellular domain for use in the invention include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability.

Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In one embodiment, the intracellular domain of the CAR includes any portion of a co-stimulatory molecule, such as at least one signaling domain from CD3, CD27, CD28, ICOS, 4-1BB, PD-1, T cell receptor (TCR), any derivative or variant thereof, any synthetic sequence thereof that has the same functional capability, and any combination thereof.

Between the antigen binding domain and the transmembrane domain of the CAR, or between the intracellular domain and the transmembrane domain of the CAR, a spacer domain may be incorporated. As used herein, the term “spacer domain” generally means any oligo- or polypeptide that functions to link any domain, such as linking the transmembrane domain to, either the antigen binding domain or, the intracellular domain in the polypeptide chain. The spacer domain may be on one or both ends of the CAR. In one embodiment, the spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between the transmembrane domain and the intracellular domain of the CAR. An example of a linker includes a glycine-serine doublet.

### Human Antibodies

It may be preferable to use human antibodies or fragments thereof when using bispecific antibodies or the antigen binding domains of a CAR. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The bispecific antibody can also include an antibody wherein the heavy and light chains are encoded by a nucleotide sequence derived from one or more sources of human DNA.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Humanized Antibodies

Alternatively, in some embodiments, a non-human antibody can be humanized, where specific sequences or regions of the antibody are modified to increase similarity to an antibody naturally produced in a human. For instance, in the present invention, the antibody or fragment thereof may comprise a non-human mammalian scFv. In one embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody. However, using certain methods of humanization, the affinity and/or specificity of binding of the antibody to the target antigen may be increased using methods of “directed evolution,” as described by Wu et al., J. Mol. Biol., 294:151 (1999), the contents of which are incorporated by reference herein in their entirety.

### Generating Engineered T Cells

Described herein are methods of engineering the T cells including one or more CAR T cells described herein. In some embodiments, the method can include one or more steps that generate a T cell or population thereof that expresses or is capable of expressing a CAR. In some embodiments, the method can include one or more steps that generate a T cell or population that expresses or is capable of expressing a CAR and a chimeric intracellular signaling molecule, a co-stimulatory molecule, bispecific antibody, antibody or fragment thereof, or any combination thereof. In some embodiments, the method can include one or more steps that generate a T cell or population thereof that expresses or is capable of expressing a CAR and a co-stimulatory molecule (e.g. CD28 or 4-11BB). In some aspects, the CAR comprises a CD3 domain, such as a CD3 zeta domain. In one aspect, the invention includes a method for generating a metabolically enhanced T cell. In some of these embodiments, the method can include one or more steps that generate a T cell that expresses or is capable of expressing a chimeric intracellular signaling molecule.

### Methods of Modifying T Cells

The method can include introducing a nucleic acid sequence encoding a chimeric intracellular signaling molecule, CAR, co-stimulatory molecule, antibody, bispecific antibody, or any combination thereof into a T cell, wherein the nucleic acid sequence comprises a nucleic acid sequence of an intracellular domain of a co-stimulatory molecule and substantially lacks an extracellular ligand-binding domain. At least one co-stimulatory molecule on the T cell is stimulated, which activates the chimeric intracellular signaling molecule, thereby metabolically enhancing the T cell. Methods and techniques of modifying cells, such as recombinant engineering and gene modification techniques, are generally known in the art and will be appreciated by those of ordinary skill in the art in view of this description. Exemplary methods and techniques are also described herein.

In one embodiment, the nucleic acid sequence is selected from the group consisting of a DNA and an mRNA. In another embodiment, the nucleic acid sequence is electroporated into the T cell. The nucleic acid sequence can be a vector. Examples of vectors include but are not limited to plasmid vectors, viral vectors, retrotransposons, site directed insertion vectors, and a suicide expression vector.

In some embodiments, the method includes introducing a CAR into a T cell, wherein the CAR comprises an antigen binding domain, a transmembrane domain and an intracellular domain of a co-stimulatory molecule, arming the CAR T cell with a bispecific antibody, wherein the bispecific antibody binds to a target on a tumor cell and the CAR T cell, and stimulating at least one co-stimulatory molecule on the armed CAR T cell, wherein the stimulation activates the intracellular domain of the co-stimulatory molecule thereby metabolically enhancing the armed T cell. In one embodiment, introducing the CAR into the T cell comprises introducing a nucleic acid sequence encoding the CAR, such as by electroporating a mRNA encoding the CAR. In another embodiment, arming the CAR T cell comprises contacting the CAR T cell with the bispecific antibody. In yet another embodiment, arming the CAR T cell comprises introducing a nucleic acid sequence encoding the bispecific antibody, such as by electroporating a mRNA encoding the bispecific antibody. In still another embodiment, stimulating the armed CAR T cell improves cytotoxicity and resistance to immunosuppression of the armed CAR T cell when in a tumor microenvironment. In yet another embodiment, the method further comprises irradiating the CAR T cell with up to 2500 rad to inhibit proliferation of the CAR T cell without inhibiting cytokine secretion or inducing cytotoxicity.

In certain example embodiments, provided herein are methods of modulating a CAR T cell, comprising: administering a modulating agent to a CAR T cell, wherein the modulating agent is capable of modifying the expression of one or more genes in the CAR T cell such that the CAR T cell comprises a gene signature selected from:

g) a CD3ζ CAR T gene signature,

h) a costimulatory molecule gene signature,

i) a T_{H}1 response gene signature,

j) a T_{H}2 response gene signature,

k) a T cell activation gene signature,

l) any combination thereof.

In certain example embodiments, the CD3ζ CAR T gene signature comprises one or more signature genes selected from the group consisting of: ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof. In certain example embodiments, one or more signature genes in the CD3ζ CAR T gene signature are up-regulated, down-regulated, or both. In certain example embodiments, the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof. In certain example embodiments, the CD3ζ CAR T gene signature comprises ZP3 or GGT1. In certain example embodiments, the CD3ζ CAR T gene signature comprises CCL3, CCL4, GZMB, XCL1, ZBED2, IFNG, or any combination thereof.

In certain example embodiments, the costimulatory molecule gene signature comprises one or more signature genes of Table 7, Table 8, or any combination thereof. In certain example embodiments, one or more signature genes in the costimulatory molecule gene signature are up-regulated, down-regulated, or both. In certain example embodiments, the costimulatory molecule gene signature comprises a gene signature selected from the group consisting of:

(a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6, ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
(b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
(c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, and any combination thereof;
(d) JUN, GPA33, KRT1, EGR1, CIITA, UBD, KLHL23, SCD, HLA-DOA, ALPK, CXCL10, and any combination thereof;
(e) JUN, EGR1, CIITA, GPA33, KRT1, and any combination thereof;
(f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
(g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
(h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
(i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
(j) CXCL10, JUNB, NTRK2, MSC, VNN2 and any combination thereof;
(k) JUNB, CXCL10, ENOX1, ENPP2, DDIT4, NTRK2, GCSAM, IL5, and any combination thereof;

(l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;

(m) ENPP2, GJB2, C4orf26, MX1, NTRK2, JUNB, TNFRSF8, DGAT2, ELL2, IL4I1, ITPR1, HLA-DRB6, and any combination thereof, and
(n) CIITA, CD74, HLA-DMB, HLA-DPB1, HLA-DQA2, HLA-DRB1, HLA-DRB5, HLA-DOA, HLA-DRA, HLA-DRB6, and any combination thereof.

In certain example embodiments, the gene signature is any one of gene signatures (a)-(i). In certain example embodiments, the gene signature is any one of gene signatures (a), (b), (c), (j), (k), (l), or (m). In certain example embodiments, the gene signature is any one of gene signatures (a), (d), (e), or (j). In certain example embodiments, the gene signature is any one of gene signatures (b), (c), (f), (g), (h), (i), (k), (l), (m). In certain example embodiments, one or more genes in any one of gene signatures (a)-(i) is overexpressed, underexpressed, or both as compared to an unmodified CAR T cell. In certain example embodiments, LGMN, PDCD1, GPA33, KRT1, VNN2, C17orf-PLSCR3, and any combination thereof is overexpressed in the CART cell. IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is underexpressed in the CART cell. In certain example embodiments, IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is overexpressed in the CAR T cell. In certain example embodiments, LGMN, PDCD1, GPA33, KRT1, VNN2, C17orf-PLSCR3, and any combination thereof is underexpressed in the CART cell.

In certain example embodiments, the T_{H}1 response gene signature comprises one or more signature genes selected from the group consisting of: ERG1, TBX21, RORC, IL12RB2, GLIL1, EPPN2, DMD, IFNG, and any combination thereof.

In certain example embodiments, the T_{H}2 response gene signature comprises one or more signature genes selected from the group consisting of: IL4, IL5, IL2, and any combination thereof.

In certain example embodiments, the T cell activation gene signature comprises one or more genes selected from Table 3, Table 4, or a combination thereof.

In certain example embodiments, the T cell activation gene signature comprises one or more genes selected from the group consisting of: IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof. In certain example embodiments, IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are overexpressed or underexpressed in the CAR T cell. In certain example embodiments, the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1 TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

In certain example embodiments, the modifying agent is a therapeutic antibody, antibody fragment, antibody-like protein scaffold, aptamer, polypeptide, protein, genetic modifying agent, small molecule, small molecule degrader, or combination thereof. In certain example embodiments, the genetic modifying agent is a CRISPR-Cas system, a TALEN, a Zn-finger nuclease, or a meganuclease.

In certain example embodiments, provided herein are isolated or engineered CAR T cells obtained according to any method described herein such as those in numbered aspects 1-72.

### Vectors

A vector may be used to introduce the chimeric intracellular signaling molecule, CAR, or other molecule or gene as desired into a T cell as described elsewhere herein. In one aspect, the invention includes a vector comprising a nucleic acid sequence encoding a chimeric intracellular signaling molecule and, optionally, a nucleic acid sequence encoding a bispecific antibody as described herein. In another aspect, the invention includes a vector comprising a nucleic acid sequence encoding a CAR and, optionally, a nucleic acid sequence encoding a bispecific antibody as described herein. In one embodiment, the vector comprises a plasmid vector, viral vector, retrotransposon (e.g. piggyback, sleeping beauty), site directed insertion vector (e.g. CRISPR, zn finger nucleases, TALEN), or suicide expression vector, or other known vector in the art.

All constructs mentioned above are capable of use with 3rd generation lentiviral vector plasmids, other viral vectors, or RNA approved for use in human cells. In one embodiment, the vector is a viral vector, such as a lentiviral vector. In another embodiment, the vector is a RNA vector.

The production of any of the molecules described herein can be verified by sequencing. Expression of the full-length proteins may be verified using immunoblot, immunohistochemistry, flow cytometry or other technology well known and available in the art.

The present invention also provides a vector in which DNA of the present invention is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids is typically achieved by operably linking a nucleic acid or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

The nucleic acid can be cloned into any number of different types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY, and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-la promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to, a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

Methods of introducing and expressing genes, such as the chimeric intracellular signaling molecule or the CAR, into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY. Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001)).

Biological methods for introducing a polynucleotide of interest into a host cell include the use of DNA and RNA vectors. RNA vectors include vectors having a RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, one or more of the nucleic acid sequences described elsewhere herein are introduced by a method selected from the group consisting of transducing the population of cells, transfecting the population of cells, and electroporating the population of cells. In one embodiment, a population of cells comprises one or more of the nucleic acid sequences described herein.

In one embodiment, the nucleic acids introduced into the cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a chimeric intracellular signaling molecule and/or a bispecific antibody.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5′, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

Some in vitro-transcribed RNA (IVT-RNA) vectors are known in the literature which are utilized in a standardized manner as template for in vitro transcription and which have been genetically modified in such a way that stabilized RNA transcripts are produced. Currently, protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In one aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Generating Metabolically Enhanced T Cells

In one aspect, the invention includes a method for generating a metabolically enhanced T cell. The method comprises introducing a nucleic acid sequence encoding a chimeric intracellular signaling molecule into a T cell, wherein the nucleic acid sequence comprises a nucleic acid sequence of an intracellular domain of a co-stimulatory molecule and substantially lacks an extracellular ligand-binding domain. At least one co-stimulatory molecule on the T cell is stimulated, which activates the chimeric intracellular signaling molecule, thereby metabolically enhancing the T cell.

In one embodiment, the nucleic acid sequence is selected from the group consisting of a DNA and an mRNA. In another embodiment, the nucleic acid sequence is electroporated into the T cell. The nucleic acid sequence can be a vector. Examples of vectors include, but are not limited to, plasmid vectors, viral vectors, retrotransposons, site directed insertion vectors, and suicide expression vectors.

In certain embodiments, the method for generating a metabolically enhanced T cell further comprises arming the T cell with a bispecific antibody, wherein the bispecific antibody is displayed on the T cell surface. In one embodiment, arming the T cell comprises contacting the T cell with the bispecific antibody. In another embodiment, the bispecific antibody specifically binds the T cell. In yet another embodiment, the T cell is armed with two or more bispecific antibodies, and the T cell displays the two or more bispecific antibodies. In still another embodiment, the two or more bispecific antibodies specifically bind the T cell. The bispecific antibodies can comprise a combination of antibodies selected from the group consisting of anti-CD3, anti-IgD Fc, and anti-IgA Fc. In certain embodiments, the bispecific antibody is chemically heterconjugated to a polyclonal antibody specific for a tumor-associated antigen (TAA), and the T cell specifically binds the TAA polyclonal antibody.

In certain embodiments, arming the cell comprises electroporating a nucleic acid sequence encoding a bispecific antibody. The bispecific antibody comprises a first antigen binding domain that binds to a first antigen and a second antigen binding domain that binds to a second antigen. In one embodiment, the bispecific antibody comprises a first antigen binding domain that binds to a target cell and a second antigen binding domain that binds to an activated T cell. In another embodiment, the bispecific antibody comprises an antigen binding domain comprising a first and a second single chain variable fragment (scFv) molecule. In yet another embodiment, the bispecific antibody comprises an antigen binding domain comprising a first whole immunoglobulin molecule and a second whole IgG immunoglobulin molecule. At least one of the first or second whole immunoglobulin molecules can be IgG, IgA, or IgD.

Alternatively, in another aspect, the invention includes a method for generating a modified T cell comprising electroporating a population of T cells with a nucleic acid sequence encoding a chimeric intracellular signaling molecule, wherein the nucleic acid sequence comprises a nucleic acid sequence of an intracellular domain of a co-stimulatory molecule and substantially lacks an extracellular ligand-binding domain. In one embodiment, the nucleic acid sequence encoding a chimeric intracellular signaling molecule is electroporated into a cell. In another embodiment, a nucleic acid sequence encoding a bispecific antibody is further electroporated into the cell. In yet another embodiment, a nucleic acid sequence encoding a CAR is further electroporated into the cell.

Alternatively, the invention includes a method of metabolically enhancing a tumor specific T cell, comprising introducing a CAR into a T cell, wherein the CAR comprises an antigen binding domain, a transmembrane domain and an intracellular domain of a co-stimulatory molecule, arming the CAR T cell with a bispecific antibody, wherein the bispecific antibody binds to a target on a tumor cell and the CAR T cell, and stimulating at least one co-stimulatory molecule on the armed CAR T cell, wherein the stimulation activates the intracellular domain of the co-stimulatory molecule thereby metabolically enhancing the armed T cell. In one embodiment, introducing the CAR into the T cell comprises introducing a nucleic acid sequence encoding the CAR, such as by electroporating a mRNA encoding the CAR. In another embodiment, arming the CAR T cell comprises contacting the CAR T cell with the bispecific antibody. In yet another embodiment, arming the CAR T cell comprises introducing a nucleic acid sequence encoding the bispecific antibody, such as by electroporating a mRNA encoding the bispecific antibody. In still another embodiment, stimulating the armed CAR T cell improves cytotoxicity and resistance to immunosuppression of the armed CAR T cell when in a tumor microenvironment. In yet another embodiment, the method further comprises irradiating the CAR T cell with up to 2500 rad to inhibit proliferation of the CAR T cell without inhibiting cytokine secretion or inducing cytotoxicity.

### Genetic Modifications

The immune cells, such as T cells, can be modified using any suitable recombinant engineering method or technique that will be appreciated by those of ordinary skill in the art. Exemplary techniques are described herein and can be applied to modifying any of the cells described elsewhere herein. Suitable genetic modifying agents for modifying one or more immune cells (e.g. T cells) to generate the engineered T cells described herein include, but are not limited to, CRISPR-Cas systems, TALENs, meganucleases, zinc finger nuclease systems, and the like.

In certain preferred embodiments, the cell may be modified edited using any CRISPR-based system generally known in the art and those specifically described herein. In certain preferred embodiments, cells are edited or otherwise modified ex vivo and transferred to a subject in need thereof. This is also referred to as adoptive cell therapy, which is described in greater detail elsewhere herein.

Further genetically modifying, such as gene editing, of the cell may be performed for example (1) to insert or knock-in an exogenous gene, such as an exogenous gene encoding a CAR or a TCR, antibody (including bispecific antibodies), co-stimulatory molecule, chimeric intracellular molecule, at a preselected locus in the cell; (2) to knock-out or knock-down expression of an endogenous TCR in the cell; (3) to disrupt the target of a chemotherapeutic agent in the cell; (4) to knock-out or knock-down expression of an immune checkpoint protein or receptor in the cell; (5) to knock-out or knock-down expression of other gene or genes in the cell, the reduced expression or lack of expression of which can enhance the efficacy of adoptive therapies using the cell; (6) to knock-out or knock-down expression of an endogenous gene in a cell, said endogenous gene encoding an antigen targeted by an exogenous CAR or TCR; (7) to knock-out or knock-down expression of one or more MHC constituent proteins in the cell; (8) to activate a T cell, and/or increase the differentiation and/or proliferation of functionally exhausted or dysfunctional CD8⁺ T cells; and/or (9) to modulate CD8⁺ T cells, such that CD8⁺ T cells have increased resistance to exhaustion or dysfunction. In certain preferred embodiments, the cell may be edited to produce any one of the following combinations of the modifications set forth above: (1) and (2); (1) and (4); (2) and (4); (1), (2) and (4); (1) and (7); (2) and (7); (4) and (7); (1), (2) and (7); (1), (4) and (7); (1), (2), (4) and (7); optionally adding modification (8) or (9) to any one of the preceding combinations. In certain preferred embodiments, the targeted immune checkpoint protein or receptor is PD-1, PD-L1 and/or CTLA-4. In certain preferred embodiments, the targeted endogenous TCR gene or sequence may be TRBC1, TRBC2 and/or TRAC. In certain preferred embodiments, the targeted MHC constituent protein may be HLA-A, B and/or C, and/or B2M. In certain embodiments, the cell may thus be multiply edited (multiplex genome editing) to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Arming T Cells with Bispecific Antibodies

In certain embodiments, the method for generating a T cell described herein further comprises arming the T cell with a bispecific antibody, wherein the bispecific antibody is displayed on the T cell surface. In one embodiment, arming the T cell comprises contacting the T cell with the bispecific antibody. In another embodiment, the bispecific antibody specifically binds the T cell. In yet another embodiment, the T cell is armed with two or more bispecific antibodies, and the T cell displays the two or more bispecific antibodies. In still another embodiment, the two or more bispecific antibodies specifically bind the T cell. The bispecific antibodies can comprise a combination of antibodies selected from the group consisting of anti-CD3, anti-IgD Fc, and anti-IgA Fc. In certain embodiments, the bispecific antibody is chemically heterconjugated to a polyclonal antibody specific for a tumor-associated antigen (TAA), and the T cell specifically binds the TAA polyclonal antibody.

In certain embodiments, arming the cell comprises electroporating a nucleic acid sequence encoding a bispecific antibody. The bispecific antibody comprises a first antigen binding domain that binds to a first antigen and a second antigen binding domain that binds to a second antigen. In one embodiment, the bispecific antibody comprises a first antigen binding domain that binds to a target cell and a second antigen binding domain that binds to an activated T cell. In another embodiment, the bispecific antibody comprises an antigen binding domain comprising a first and a second single chain variable fragment (scFv) molecule. In yet another embodiment, the bispecific antibody comprises an antigen binding domain comprising a first whole immunoglobulin molecule and a second whole IgG immunoglobulin molecule. At least one of the first or second whole immunoglobulin molecules can be IgG, IgA, or IgD.

### Sources of T Cells

The metabolically enhanced T cells may be generated from any source of T cells. In one embodiment, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface particles (e.g., such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, a population of cells comprise the T cells of the present invention. Examples of a population of cells include, but are not limited to, peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of T Cells

T cells generated by any method described herein may be expanded ex vivo. In one embodiment, T cells or a population of cells comprising T cells are cultured for expansion. Generally, T cells are expanded by contact with a surface having attached thereto an agent that stimulates a CD3/TCR complex associated signal and a ligand that stimulates a co-stimulatory molecule on the surface of the T cells with or without IL-2.

Methods for expanding T cells are described herein. For example, the T cells can be expanded by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial intergers therebetween. In one embodiment, the T cells expand in the range of about 20 fold to about 50 fold.

The T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing one or more of the molecules described elsewhere herein into the T cells.

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

In one embodiment, the T cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The T cell culturing medium may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

### Therapy

The metabolically enhanced T cells and other T cells described herein are useful in a variety of treatment modalities for treatment of a number of disease states whether the T cell is metabolically enhanced by virture of expression of either a chimeric intracellular signaling molecule or a CAR. Thus, irrespective of whether the T cell expresses a chimeric intracellular signaling molecule or a CAR, the T cell is referred to herein as a metabolically enhanced T cell. A composition comprising a metabolically enhanced T cell can be generated according to the methods described elsewhere herein. This metabolically enhanced T cell may be included in a composition for therapy as now described.

In certain example embodiments, provided herein are methods of treating a disease in a subject in need thereof comprising: administering an identified candidate cell obtained by the method as in any one of numbered aspects 1-44 or an isolated or engineered CAR T cell as in numbered aspect 73, or a cell population thereof to the subject. In certain example embodiments, the disease is a cancer. In certain example embodiments, the method can further comprise administering an additional agent, therapy, antineoplastic or antitumor agent or radiation and/or surgical therapy or an antigen or a neoantigen. In certain example embodiments, the additional agent, therapy, antineoplastic or antitumor agent or radiation and/or surgical therapy or an antigen or neoantigen is administered sequentially or concurrently. In certain example embodiments, the sequential administration comprises a time period of a day, two days, three days, four days, five days, six days, a week, two weeks, three weeks, four weeks, five weeks, six weeks, seven weeks, eight weeks, nine weeks, ten weeks, eleven weeks, twelve weeks, or more.

In certain example embodiments, provided herein are methods of screening for one or more agents capable of modifying a gene expression signature of a CAR T cell as in any one of numbered aspects 45-72, comprising: contacting an unmodified CAR T cell population with a test modulating agent or a library of modulating agents; identifying candidate CAR T cells present in the CART T cell population by the method of any one of numbered aspects 1-44; and selecting modulating agents that result in increasing the number of candidate CAR T cells present in the CAR T cell population.

In certain example embodiments, the CAR T cell or population thereof is obtained from or derived from a subject to be treated.

In one aspect, the composition comprises the metabolically enhanced T cell comprising the chimeric intracellular signaling molecule described herein. In another aspect, the composition comprises the metabolically enhanced cell further comprising the bispecific antibody described herein. The composition may include a pharmaceutical composition and further include a pharmaceutically acceptable carrier. A therapeutically effective amount of the pharmaceutical composition comprising the modified cells may be administered.

In one aspect, the invention includes a method of treating a disease or condition associated with enhanced immunity in a subject comprising administering to the subject a therapeutically effective amount of a pharmaceutical composition comprising the metabolically enhanced T cell described herein. In another aspect, the invention includes a method of treating a condition in a subject, comprising administering to the subject a therapeutically effective amount of a pharmaceutical composition comprising the metabolically enhanced T cell described herein. In another aspect, the invention includes a method for stimulating a T cell-mediated immune response to a target cell or tissue in a subject comprising administering to a subject a therapeutically effective amount of a pharmaceutical composition comprising the metabolically enhanced T cell described herein. In yet another aspect, the invention includes use of the metabolically enhanced T cell described herein in the manufacture of a medicament for the treatment of an immune response in a subject in need thereof. In these embodiments, the T cell comprises a chimeric intracellular signaling molecule, wherein the chimeric intracellular signaling molecule comprises an intracellular domain of a co-stimulatory molecule and substantially lacks an extracellular ligand-binding domain. In another embodiment, the T cell further comprises a bispecific antibody. In yet another embodiment, the T cell further comprises a CAR.

In one aspect, the invention includes a method of treating a disease or condition associated with a tumor or cancer in a subject comprising administering to the subject a therapeutically effective amount of a pharmaceutical composition comprising the metabolically enhanced T cell described herein. In another aspect, the invention includes a method of treating a solid tumor in a subject, comprising administering to the subject a therapeutically effective amount of a pharmaceutical composition comprising the metabolically enhanced T cell described herein. In another aspect, the invention includes a method for stimulating a T cell-mediated immune response to a target tumor cell or tumor tissue in a subject comprising administering to a subject a therapeutically effective amount of a pharmaceutical composition comprising the metabolically enhanced T cell described herein. In yet another aspect, the invention includes use of the metabolically enhanced T cell described herein in the manufacture of a medicament for the treatment of a tumor or cancer in a subject in need thereof. In these embodiments, the T cell comprises a CAR and a bispecific antibody, wherein the CAR comprises an antigen binding domain, a transmembrane domain and an intracellular domain of a co-stimulatory molecule, and the bispecific antibody binds to a target on a tumor cell and the T cell.

The metabolically enhanced T cells as described herein can be administered to an animal, preferably a mammal, even more preferably a human, to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the metabolically enhanced T cells of the present invention can be used for the treatment of any condition in which a diminished or otherwise inhibited immune response, especially a cell-mediated immune response, is desirable to treat or alleviate the disease. In one aspect, the invention includes treating a condition, such as an autoimmune disease, in a subject, comprising administering to the subject a therapeutically effective amount of a pharmaceutical composition comprising a population of the cells described herein.

Examples of autoimmune disease include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis, chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The metabolically enhanced T cells described herein may also be used to treat inflammatory disorders. Examples of inflammatory disorders include, but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

The metabolically enhanced T cells of the present invention can be used to treat cancers. Cancers include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The metabolically enhanced T cells of the invention can be administered in dosages and routes and at times to be determined in appropriate pre-clinical and clinical experimentation and trials. Cell compositions may be administered multiple times at dosages within these ranges. Administration of the metabolically enhanced T cells of the invention may be combined with other methods useful to treat the desired disease or condition as determined by those of skill in the art.

The metabolically enhanced T cells of the invention may be autologous, allogeneic or xenogeneic with respect to the subject administered to therein that is undergoing therapy.

The administration of the metabolically enhanced T cells of the invention may be carried out in any convenient manner known to those of skill in the art. The metabolically enhanced T cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the metabolically enhanced T cells of the invention are injected directly into a site of inflammation in the subject, a local disease site in the subject, a lymph node, an organ, a tumor, and the like.

### Pharmaceutical Compositions

Pharmaceutical compositions of the present invention may comprise the metabolically enhanced T cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer the metabolically enhanced T cells to a subject and then subsequently redraw blood (or have an apheresis performed), metabolically enhance T cells therefrom according to the present invention, and reinfuse the patient with these metabolically enhanced T cells. This process can be carried out multiple times every few weeks. In certain embodiments, metabolically enhanced T cells can be obtained from blood draws from about 10 ml to about 400 ml. In certain embodiments, metabolically enhanced T cells are obtained from blood draws of about 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of T cells.

In certain embodiments of the present invention, T cells that are metabolically enhanced using the methods described herein, and stimulated, activated or expanded using the methods described herein or other methods known in the art where T cells are expanded to therapeutic levels, are administered to a patient in conjunction with (e.g., before, simultaneously or following) any number of relevant treatment modalities including, but not limited to, treatment with agents such as antiviral therapy, cidofovir and interleukin-2, Cytarabine (also known as ARA-C) or natalizumab treatment for MS patients or treatments for PML patients. In further embodiments, the metabolically enhanced T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the metabolically enhanced T cell compositions of the present invention are administered to a patient in conjunction with (e.g., before, simultaneously or following) bone marrow transplantation, T cell ablative therapy using either chemotherapy agents such as fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day, although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the claimed invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook et al., (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel, et al., Current Protocols; 5 edition, Nov. 5, 2002); “Polymerase Chain Reaction: Principles, Applications and Troubleshooting”, (Babar, M., VDM Verlag Dr. Müller, Aug. 17, 2011); “Current Protocols in Immunology” (Coligan, John Wiley & Sons, Inc., Nov. 1, 2002). These techniques are applicable to the production of the polynucleotides and polypeptides of the invention, and, as such, may be considered in making and practicing the invention. Particularly useful techniques for particular embodiments will be discussed in the sections that follow.

### Cancer Associated Antigens

The present invention provides immune effector cells (e.g., T cells, NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs of the instant invention: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

Accordingly, the present invention provides CARs that target the following cancer associated antigens (tumor antigens): CD19, CD123, CD22, CD30, CD171, CS-1, CLL-1 (CLECL1), CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-ab1, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

### Tumor Supporting Antigens

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

Chimeric antigen receptor (CAR) T cells have emerged as a novel form of treatment of patients with B-cell malignancies. In particular, anti-CD19 CAR T-cell therapy has effected impressive clinical responses in B-cell acute lymphoblastic leukemia and diffuse large B-cell lymphoma. However, not all patients respond, and relapse with antigen loss has been observed in all patient subsets. In Blood 2018 Oct. 4; 132(14):1495-1506, the authors report on the design and optimization of a novel CAR directed to the surface antigen CD37, which is expressed in B-cell non-Hodgkin lymphomas, in chronic lymphocytic leukemia, and in some cases of cutaneous and peripheral T-cell lymphomas. The authors found that CAR-37 T cells demonstrated antigen-specific activation, cytokine production, and cytotoxic activity in models of B- and T-cell lymphomas in vitro and in vivo, including patient-derived xenografts. Taken together, these results are the first showing that T cells expressing anti-CD37 CAR have substantial activity against 2 different lymphoid lineages, without evidence of significant T-cell fratricide. Furthermore, anti-CD37 CARs were readily combined with anti-CD19 CARs to generate dual-specific CAR T cells capable of recognizing CD19 and CD37 alone or in combination. The findings indicate that CD37-CAR T cells represent a novel therapeutic agent for the treatment of patients with CD37-expressing lymphoid malignancies.

Adoptive cell therapy (ACT) with antigen-specific T cells has shown remarkable clinical success; however, approaches to safely and effectively augment T cell function, especially in solid tumors, remain of great interest. In Nat Biotechnol. 2018 September; 36(8):707-716, the authors describe a strategy to ‘backpack’ large quantities of supporting protein drugs on T cells by using protein nanogels (NGs) that selectively release these cargos in response to T cell receptor activation. The authors designed cell surface-conjugated NGs that responded to an increase in T cell surface reduction potential after antigen recognition and limited drug release to sites of antigen encounter, such as the tumor microenvironment. By using NGs that carried an interleukin-15 super-agonist complex, it has been demonstrated that, relative to systemic administration of free cytokines, NG delivery selectively expanded T cells 16-fold in tumors and allowed at least eightfold higher doses of cytokine to be administered without toxicity. The improved therapeutic window enabled substantially increased tumor clearance by mouse T cell and human chimeric antigen receptor (CAR)-T cell therapy in vivo.

Cancer has an impressive ability to evolve multiple processes to evade therapies. While immunotherapies and vaccines have shown great promise, particularly in certain solid tumors such as prostate cancer, they have been met with resistance from tumors that use a multitude of mechanisms of immunosuppression to limit effectiveness. Prostate cancer, in particular, secretes transforming growth factor β (TGF-β) as a means to inhibit immunity while allowing for cancer progression. Blocking TGF-βsignaling in T cells increases their ability to infiltrate, proliferate, and mediate antitumor responses in prostate cancer models. In Mol Ther. 2018 Jul. 5; 26(7):1855-1866, the authors tested whether the potency of chimeric antigen receptor (CAR) T cells directed to prostate-specific membrane antigen (PSMA) could be enhanced by the co-expression of a dominant-negative TGF-βRII (dnTGF-βRII). Upon expression of the dominant-negative TGF-βRII in CAR T cells, the authors observed increased proliferation of these lymphocytes, enhanced cytokine secretion, resistance to exhaustion, long-term in vivo persistence, and the induction of tumor eradication in aggressive human prostate cancer mouse models. Based on the observations, the authors initiated a phase I clinical trial to assess these CAR T cells as a novel approach for patients with relapsed and refractory metastatic prostate cancer.

In Sci Transl Med. 2017 Jul. 19; 9(399), the authors conducted a first-in-human study of intravenous delivery of a single dose of autologous T cells redirected to the epidermal growth factor receptor variant III (EGFRvIII) mutation by a chimeric antigen receptor (CAR). The authors found that manufacturing and infusion of CAR-modified T cell (CART)-EGFRvIII cells are feasible and safe, without evidence of off-tumor toxicity or cytokine release syndrome. One patient has had residual stable disease for over 18 months of follow-up. All patients demonstrated detectable transient expansion of CART-EGFRvIII cells in peripheral blood. Seven patients had post-CART-EGFRvIII surgical intervention, which allowed for tissue-specific analysis of CART-EGFRvIII trafficking to the tumor, phenotyping of tumor-infiltrating T cells and the tumor microenvironment in situ, and analysis of post-therapy EGFRvIII target antigen expression. Imaging findings after CART immunotherapy were complex to interpret, further reinforcing the need for pathologic sampling in infused patients. The authors found trafficking of CART-EGFRvIII cells to regions of active GBM, with antigen decrease in five of these seven patients. In situ evaluation of the tumor environment demonstrated increased and robust expression of inhibitory molecules and infiltration by regulatory T cells after CART-EGFRvIII infusion, compared to pre-CART-EGFRvIII infusion tumor specimens. The initial experience with CAR T cells in recurrent GBM suggests that although intravenous infusion results in on-target activity in the brain, overcoming the adaptive changes in the local tumor microenvironment and addressing the antigen heterogeneity may improve the efficacy of EGFRvIII-directed strategies in GBM.

Cytokine release syndrome (CRS) is a potentially severe systemic toxicity seen after adoptive T-cell therapy and caused by T-cell activation and proliferation and is associated with elevated circulating levels of cytokines such as C-reactive protein, interleukin-6 (IL-6), and interferon-γ and has previously been described as a systemic response in hematologic malignancies. As described in J Immunother. 2017 April; 40(3):104-107, a 52-year-old woman with BRCA 1 mutation positive heavily pretreated advanced recurrent serous ovarian cancer was treated under a compassionate use protocol with autologous mesothelin-redirected chimeric antigen receptor T cells (CART-meso). Autologous T cells were transduced to express a receptor composed of an extracellular antimesothelin single-chain variable fragment fused to 4-1BB and TCR-zeta signaling domain. This patient was infused with 3×10 CART-meso T cells/m without lymphodepletion and developed compartmental CRS confined to the pleural cavities. The compartmental CRS was evidenced by an increase in IL-6 and accumulation of CART-meso T cells in pleural fluid compared with peripheral blood and was successfully treated the anti-IL6 receptor antagonist tocilizumab on D21 after the T-cell infusion. This is the first description of a compartmental CRS in a patient with solid malignancy. This response could be due to malignant pleural fluid creating an environment where T cells could interact with tumor cells and suggests localized on-target CAR-T-cell activation.

Chimeric antigen receptors (CARs) are synthetic receptors that usually redirect T cells to surface antigens independent of human leukocyte antigen (HLA). In Mol Ther Oncolytics. 2017 Jan. 11; 3:1-9, the authors investigated a T cell receptor-like CAR based on an antibody that recognizes HLA-A*0201 presenting a peptide epitope derived from the cancer-testis antigen NY-ESO-1. The authors hypothesized that this CAR would efficiently redirect transduced T cells in an HLA-restricted, antigen-specific manner. However, the authors found that despite the specificity of the soluble Fab, the same antibody in the form of a CAR caused moderate lysis of HLA-A2 expressing targets independent of antigen owing to T cell avidity. The authors hypothesized that lowering the affinity of the CAR for HLA-A2 would improve its specificity. The authors undertook a rational approach of mutating residues that, in the crystal structure, were predicted to stabilize binding to HLA-A2. The authors found that one mutation (DN) lowered the affinity of the Fab to T cell receptor-range and restored the epitope specificity of the CAR. DN CAR T cells lysed native tumor targets in vitro, and, in a xenogeneic mouse model implanted with two human melanoma lines (A2+/NYESO+ and A2+/NYESO−), DN CAR T cells specifically migrated to, and delayed progression of, only the HLA-A2+/NY-ESO-1+ melanoma. Thus, although maintaining MHC-restricted antigen specificity required T cell receptor-like affinity that decreased potency, there is exciting potential for CARs to expand their repertoire to include a broad range of intracellular antigens.

Reference is made to International Patent Publication WO2018191748 which provides chimeric antigen receptor (CAR) T cells including a heterologous nucleic acid molecule, wherein the heterologous nucleic acid molecule includes: (a) a first polynucleotide encoding a CAR including an antigen-binding domain, a transmembrane domain, and an intracellular signaling domain; and (b) a second polynucleotide encoding a therapeutic agent. Optionally, the first and second polynucleotides are included within a single polynucleotide molecule. Furthermore, in some embodiments, the CAR further includes one or more co-stimulatory domains. In various embodiments, the therapeutic agent is or includes an antibody reagent (e.g., a single chain antibody, a single domain antibody (e.g., a camelid antibody), or a bispecific antibody reagent (e.g., a bispecific T cell engager (BiTE)). In other embodiments, the therapeutic agent is or includes a cytokine. In various embodiments, the CAR and the therapeutic agent are produced in the form of a polyprotein (and thus may be encoded within a single nucleic acid molecule), which is cleaved to generate separate CAR and therapeutic agent molecules. In some embodiments, the polyprotein includes a cleavable moiety (e.g., a 2A peptide, such as P2A or T2A) between the CAR and the therapeutic agent. In some embodiments, the CAR and the therapeutic agent are each constitutively expressed. In some embodiments, expression of the CAR and the therapeutic agent is driven by an elongation factor-1 alpha (EF1 α) promoter. In some embodiments, the therapeutic agent is expressed under the control of an inducible promoter (e.g., the NFAT promoter), which is optionally inducible by T cell receptor or CAR signaling. In some embodiments, the CAR is expressed under the control of a constitutive promoter and the therapeutic agent is expressed under the control of an inducible promoter (e.g., the NFAT promoter), which is optionally inducible by T cell receptor or CAR signaling. In various embodiments, the antigen-binding domain of the CAR is or includes an antibody, a single chain antibody, a single domain antibody (e.g., a camelid antibody), or a ligand. In various embodiments, the CAR antigen-binding domain or the therapeutic agent, when the therapeutic agent is or includes an antibody reagent, bind to a tumor-associated antigen. In various embodiments, the tumor-associated antigen to which the CAR antigen-binding domain or the therapeutic agent binds is a solid tumor-associated antigen. In various embodiments, the tumor-associated antigen to which the CAR antigen-binding domain or the therapeutic agent binds includes epidermal growth factor receptor variant III (EGFRvlll), EGFR, CD19, prostate-specific membrane antigen (PSMA), or IL-13 receptor alpha 2 (IL-13Rα2), and optionally the CAR antigen-binding domain. In various embodiments, the CAR antigen-binding domain or the therapeutic agent, when the therapeutic agent is or includes an antibody reagent, binds to a Treg-associated antigen. In various embodiments, the Treg-associated antigen to which the CAR antigen-binding domain or the therapeutic agent binds is selected from the group consisting of glycoprotein A repetitions predominant (GARP), latency-associated peptide (LAP), CD25, and cytotoxic T lymphocyte-associated antigen-4 (CTLA-4), and optionally the CAR antigen-binding domain. The invention further provides CAR T cells including a polynucleotide encoding a CAR, wherein the CAR includes an antigen-binding domain, a transmembrane domain (e.g., CD8 hinge/TM), and an intracellular signaling domain (e.g., CD3z); and the antigen-binding domain binds to a Treg-associated antigen. In various embodiments, the Treg-associated antigen is selected from the group consisting of GARP, LAP, CD25, and CTLA-4. In various embodiments, the CAR further includes one or more co-stimulatory domains (e.g., 4-1 BB). In various examples, the antigen-binding domain of the CAR includes a scFv or a single domain antibody.

Reference is made to US Patent Publication 20180140602 which features, at least in part, compositions and methods of treating disorders as cancer (e.g., hematological cancers or other B-cell malignancies) using immune effector cells (e.g., T cells or NK cells) that express a Chimeric Antigen Receptor (CAR) molecule, e.g., a CAR that binds to a B-cell antigen, e.g., Cluster of Differentiation 19 protein (CD19) (e.g., OMIM Acc. No. 107265, Swiss Prot. Acc No. P15391). The compositions include, and the methods include administering, immune effector cells (e.g., T cells or NK cells) expressing a B cell targeting CAR, in combination with a BTK inhibitor (e.g., a compound of formula (I) or a pharmaceutically acceptable salt thereof). In some embodiments, the combination maintains or has better clinical effectiveness as compared to either therapy alone. The invention further pertains to the use of engineered cells, e.g., immune effector cells (e.g., T cells or NK cells), to express a CAR molecule that binds to a B-cell antigen, e.g., CD19, in combination with a BTK inhibitor (e.g., a BTK inhibitor described herein) to treat a disorder associated with expression of a B-cell antigen, e.g., CD19 (e.g., a cancer, e.g., a hematological cancer).

Reference is made to US Patent Publication 20170008963 which provides compositions and methods for controlling an immune response in patients by providing optimized and/or humanized antibodies or antibody fragments (e.g., scFv) that bind Epidermal Growth Factor Receptor III (EGFRvIII) integrated into a Chimeric Antigen Receptor (CAR) construct. In some embodiments, the invention pertains to the use of T cells engineered to express an antibody or antibody fragment that bind EGFRvIII, e.g., a humanized antibody or antibody fragment that binds EGFRvIII, integrated into a CAR to treat a cancer associated with expression of EGFRvIII. In some aspects, the invention pertains to adoptive cell transfer that may be particularly suitable for patients with glioma because the specificity, number, and functional phenotype of cells prepared ex vivo can be manipulated and controlled far better than native T-cells induced by in vivo immunization.

### Dysfunctional T Cells and Methods of Modulating Said Dysfunctional T Cells

T cells can be or become dysfunctional. T cell exhaustion is a type of T cell dysfunction. CAR T cells, like other T cells, can become exhausted and/or dysfunctional. Generally, dysfunctional T cells, including CAR T cells, are characterized by reduced proliferative capacity, decreased effector function, and overexpression of multiple inhibitory receptors. Described herein are methods of identifying dysfunctional T cells, including CAR T cells. Dysfunction or exhaustion of CAR T cells can contribute to reduced efficacy of the CAR T cells when used as a therapy. In some embodiments, the method includes identifying T cells, such as CAR T cells, that have a dysfunctional gene signature and/or activated state gene signature.

Identification of Gene Modules Associated with Dysfunctional T Cell State and Activated T Cell State

The present analysis identifies gene modules that are uniquely associated with the dysfunctional T cell state and activated T cell state, and key molecular nodes that control them. The present markers, marker signatures and molecular targets thus provide for new ways to evaluate and modulate immune responses, such as to specifically evaluate and target the dysfunctional T cell state while leaving T cell activation programs intact.

### Dysfunctional T Cell State

Described herein are genes and gene products differentially upregulated in T cells expressing the dysfunction module, which thus provide useful markers, marker signatures and molecular targets specifically for dysfunction in T cells. Described herein are genes and gene products differentially upregulated in T cells expressing the activation module, which thus provide useful markers, marker signatures and molecular targets specifically for activation in T cells.

Accordingly, an aspect of the invention provides a method of detecting dysfunctional immune cells comprising detection of a gene expression signature of dysfunction selected from the group consisting of:

a) a signature comprising or consisting of one or more markers selected from the group consisting of CD83, CCR8, TNFRSF4, CD74, CCR7, TNFSF11, CD81, TBC1D4, REL, PLK2, XCL1, TNFSF4, SLC2A6, AI836003, LAD1, 1700019D03RIK, BCL6, MNDA, RAMP3, GPM6B, BHLHE40, AXL, ECE1, FILIP1L, KIT, ITGB1, CCL1, NFKB2, PLXDC2, ARC, DUSP4, CD200, TRAF1, ZHX2, NCF1, CCDC28B, PTPRS, ST6GALNAC3, TUSC3, PDCD1LG2, SDHAF1, ARAP2, KLF4, E130308A19RIK, FAM46A, TNFRSF18, SYNJ2, CYTH3, TNFSF8, CD160, RPL10, CRTAM, RAB6B, PTGER2, NFKB1, ANKRD46, ST6GALNAC6, ITPR1, ITM2C, BTLA, TSPAN32, CD82, NFKBIA, MS4A4C, RARG, NRGN, TRIB1, ZC3H12D, BMYC, IFI27L2A, GADD45B, NAPSA, KLRB1F, RASGEF1A, FOSB, MAP3K8, HIVEP1, SSH1, RABGAP1L, ZFP36L1, ARL4D, CACNA1S, NFAT5, DNAJC12, SOWAHC, SDF4, TMEM120B, DUSP1, ELK3, JUNB, GRAMD1B, LIMK1, ZFC3H1, OSTF1, LTA, DNMT3A, BCL7C, TSPAN13, ASNSD1, TGIF1, NRN1, SYNGR2, MSI2, UAP1, UNC93B1, JAK2, KDM2B, ANXA5, PRDX2, TMEM173, PHACTR2, CCDC104, CEP85L, IRF5, INF2, ITGB3, MPC1, BCL2A1D, PARP3, ASAP1, MRPS6, RELB, FAM110A, GPR68, NRP1, CAPG, SCYL2, SAMD3, H2-AB1, HSF2, CD44, STX6, POLG2, TESPA1, ALCAM, NSMF, LRRC8D, HIF1A, PACSIN1, PKP4, ASS1, NR4A3, ENO3, GYPC, KIF3B, IL2RA, RAB37, SGMS1, HLCS, SEMA6D, NMRK1, SLC17A6, SLC39A1, RPS4X, CDON, ZFP445, LAG3, RPS26, PHTF2, CST3, CD9, STAT5A, ABCA3, CSF2RA, DTX3, RSPH3A, NRIP1, SDHA, PNKD, FLNB, MGRN1, SLC26A2, HMOX2, PEX16, INPP4A, TNFRSF25, IRF8, RGCC, IFITM2, TNFSF14, NSUN6, STAT3, PFKFB3, TYROBP, HTRA2, KLRI2, CTSS, ARL5C, KLHL24, SESN3, GM5424, FAS, NCOA3, FAM53B, CALCOCO1, ERGIC3, 4930523C07RIK, PCGF5, ANXA4, and HERPUD1;
b) a signature comprising or consisting of one or more markers selected from the group consisting of CD74, CCR7, TBC1D4, SLC2A6, BCL6, JAK2, PARP3, ASAP1, RELB, H2-AB1, CD44, ABCA3, PFKFB3, SESN3, FAS, 4930523C07RIK, PCGF5, TNIP1, SPRY1, NCOA7, RPLP0, SMIM8, ANTXR2, NSMCE1, DEDD, B3GNT2, CABLES1, SLAMF6, UBL3, NR4A1, ATG7, and KDM5B;
c) a signature comprising or consisting of one or more markers selected from the group consisting of CD83, CCR8, TNFRSF4, CD74, CCR7, TNFSF11, CD81, XCL1, TNFSF4, AXL, ECE1, KIT, ITGB1, CCL1, CD200, TNFRSF18, TNFSF8, CD160, PTGER2, BTLA, TSPAN32, CD82, KLRB1F, LTA, ANXA5, ITGB3, NRP1, H2-AB1, CD44, ALCAM, GYPC, IL2RA, CDON, LAG3, CD9, TNFSF14, FAS, GDI2, TNIP1, IL21R, IL18R1, H2-AA, NR4A2, IL18RAP, CD97, TNFSF9, IRAK1BP1, GABARAPL1, TRPV2, EBAG9, GRN, RAMP1, AIMP1, BSG, IFNAR1, PRKCA, TRAF3, CD96, TNFRSF9, and NR3C1;
d) a signature comprising or consisting of one or more markers selected from the group consisting of CD83, TNFRSF4, CD74, CCR7, CD81, TNFSF4, KIT, ITGB1, CD200, TNFSF8, CD160, CD82, ITGB3, CD44, ALCAM, GYPC, IL2RA, CDON, LAG3, CD9, CSF2RA, FAS, CD97, TNFSF9, BSG, IFNAR1, TRAF3, CD96, and TNFRSF9;
e) a signature comprising or consisting of one or more markers selected from the group consisting of CD83, CD81, TNFRSF4, CXCL16, IL21R, and IL18R1;
f) a signature comprising or consisting of one or more markers selected from the group consisting of REL, BCL6, MNDA, BHLHE40, NFKB2, ZHX2, KLF4, NFKB1, NFKBIA, RARG, FOSB, HIVEP1, ZFP36L1, NFAT5, ELK3, JUNB, LIMK1, TGIF1, KDM2B, IRF5, RELB, HSF2, HIF1A, NR4A3, PHTF2, STAT5A, DTX3, NRIP1, IRF8, STAT3, NCOA3, CALCOCO1, PCGF5, NFKBIE, ETV6, RNF19A, STAT4, NR4A2, NFKBIB, PER1, GTF2A1, SPRY1, TFE3, TGIF2, RORA, RPL6, EGR2, FOXP4, TBL1X, KDM4A, COPS2, FOS, DEDD, SQSTM1, NT5C, PIAS4, ZMYM2, DMTF1, AEBP2, TRPS1, SP3, HBP1, NR4A1, TLE3, RPL7, MED21, DRAP1, TCF7, CREB3L2, ZFHX2, KDM5B, and NR3C1; or
g) a signature comprising or consisting of two or more markers each independently selected from any one of the groups as defined in any one of a) to f).

In certain embodiments, the signature further comprises one or more additional markers of dysfunction. In an embodiment, the one or more additional markers of dysfunction is a co-inhibitory receptor selected from the group consisting of PD1, CTLA4, TIGIT, TIM3, LAG3, KLRC1, BTLA, NRP1, CD160, CD274, IDO, CD200, CD244, KLRD1, LAIR1, CEACAM1, KLRA7, FAS, GPR132, CD74, SLAMF6, CD5, GPR35, CD28, CD44, and PTGER4.

Another aspect of the invention provides a method for determining whether or not an immune cell has a dysfunctional immune phenotype, said method comprising determining in said immune cell the expression of the signature of dysfunction as defined above, whereby expression of the signature indicates that the immune cell has a dysfunctional immune phenotype.

A further aspect of the invention relates to a method for determining whether or not a patient would benefit from a therapy aimed at reducing dysfunction of immune cells or a therapy aimed at upregulating of an immune response, the method comprising determining, in immune cells from said patient the expression of the signature of dysfunction as defined above, whereby expression of the signature indicates the patient will benefit from the therapy; or for determining whether or not a patient would benefit from a therapy aimed at increasing dysfunction of immune cells or a therapy aimed at downregulating of an immune response, the method comprising determining, in immune cells from said patient the expression of the signature of dysfunction as defined above, whereby expression of the signature indicates the patient will likely not benefit from the therapy.

Another aspect of the invention provides a method for determining the efficacy of a treatment of a patient with a therapy, particularly immune therapy, said method comprising determining in immune cells from said patient the expression of the signature of dysfunction as defined above before and after said treatment and determining the efficacy of said therapy based thereon.

A further aspect of the invention relates to a method for determining the suitability of a compound for modulating a dysfunctional immune phenotype and/or modulating an immune response, said method comprising contacting an immune cell expressing the signature of dysfunction as defined above with said compound and determining whether or not said compound can affect the expression of the signature by said cell.

Further aspects of the invention relate to an isolated immune cell characterised in that the immune cell comprises the signature of dysfunction as defined above; to a population of said immune cells; to a composition or pharmaceutical composition comprising said immune cell or said immune cell population; and to a method for eliciting immune tolerance in a subject comprising administering to the subject said immune cell or said immune cell population or said pharmaceutical composition.

### Activated T Cell State

Another aspect of the invention provides a method of detecting activated immune cells comprising detection of a gene expression signature of activation selected from the group consisting of:

a) a signature comprising or consisting of one or more markers selected from the group consisting of NUSAP1, CCNA2, NEIL3, SPC25, HIST1H2AB, CKAP2L, PLK1, HIST2H3C2, MXD3, FAM64A, BUB1, FOXM1, HIST2H3B, KIF22, CENPE, SKA1, CCNF, CDCA3, ESPL1, CASC5, PBK, KIF2C, SGOL1, CDK1, SHCBP1, ASPM, FBXO5, MIS18BP1, SPAG5, KIF4, ASF1B, BUB1B, AURKB, NCAPG, DEPDC1A, ESCO2, CDCA2, BC030867, KIF20A, HIST1H2AK, SMC2, ECT2, RRM2, MKI67, 2810417H13RIK, CIT, GTSE1, NCAPG2, NCAPH, CDCA8, SAPCD2, NEK2, CEP55, CDCA5, TOP2A, CCNB2, MASTL, ARHGAP19, AURKA, KIF23, CCNB1, RAD51, TACC3, MELK, STMN1, HIST1H2AE, HIST1H4D, CENPH, HIST1H1B, CDC25C, CCDC34, CDC20, KIF11, ARHGAP11A, 4930427A07RIK, FAM83D, INCENP, MAD2L1, HIST1H2AO, CKAP2, NDC80, RAD51AP1, NUF2, E2F7, CKS1B, NCAPD2, GEN1, ANLN, TICRR, POLQ, PRC1, TTK, RAD54B, E2F8, STIL, KIF18A, PARPBP, CDC45, BIRC5, KIF15, SKA2, KIF20B, TK1, PLK4, FANCD2, CENPM, C330027C09RIK, HIST2H4, SKA3, RRM1, TROAP, RAD54L, KNTC1, ZWILCH, CLSPN, TPX2, CMC2, TCF19, MCM10, HMGB2, HIST1H3C, HIST1H1E, UBE2T, CHTF18, TUBA1B, TUBB5, H2AFX, GPSM2, SPC24, UHRF1, TRIP13, PMF1, ZFP367, RACGAP1, CDC25B, UBE2C, CDKN3, CENPI, HIST1H3B, KPNA2, HJURP, BRCA1, WDR62, CENPN, GMNN, POC1A, TMPO, KNSTRN, FANCI, CENPF, 6430706D22RIK, CKS2, DIAP3, WDR67, FIGNL1, BRCA2, HMGB3, MYBL2, PKMYT1, TRAIP, RFC5, CEP128, POLA1, ANKLE1, HMGB1, FEN1, H2AFZ, TUBB4B, CTC1, CKAP5, CDC6, LIG1, POLE, MCM7, RFWD3, HMGN2, TYMS, CENPP, NCAPD3, SUV39H1, A730008H23RIK, TUBA1C, EME1, EXO1, PTMA, BLM, ULBP1, 1190002F15RIK, CDC7, DLGAP5, TUBG1, PRIM2, TMEM48, NRM, CENPA, BARD1, HAUS4, RCC1, LMNB1, and HIST1H2AG;
b) a signature comprising or consisting of one or more markers selected from the group consisting of HIST1H1E, HMGB1, HAUS4, RCC1, HAUS5, REEP4, SLBP, FKBP2, ARSB, HIST1H3E, RAD18, RAD50, TAF6, ANAPC5, FANCG, CTCF, TONSL, LMNB2, SEPHS1, HNRNPA2B1, ANAPC15, STARD3NL, 2700029M09RIK, DPY30, SDF2L1, RDM1, CDCA7L, MEAF6, MYEF2, DNAAF2, POLR3K, IPP, TARDBP, GPAA1, KPNB1, FTSJD2, RPRD1B, HIST1H1C, DPYSL2, TAF12, ARPP19, TMCO1, EXOC4, ASRGL1, CPSF6, EIF2D, CCNH, MYG1, VMA21, TFIP11, NDUFAB1, NUP35, GRPEL1, C1D, GBP3, CYB5B, PPM1G, SRSF10, PIGF, KLRE1, COG4, PDIA4, CDT1, DUSP19, ACAT2, COMMD3, HCFC2, LMAN1, ABHD10, TIMM50, CALU, AP1M1, GGH, GCDH, MRPS33, TAX1BP3, DYNLT1C, ERP44, TMEM129, COG2, TMEM167, RBX1, 0610009O20RIK, PSMB9, PUF60, LSM4, PEX19, NTAN1, ELP2, AKR1B3, PHF11B, PQLC3, ZFP142, PRADC1, TMEM209, DYNC1LI1, FARSB, MTHFSD, 1700052N19RIK, SARS2, LAMTOR1, ALDH3A2, EHBP1L1, D16ERTD472E, CCDC127, COMMD10, DNAJC14, MRPL16, SSBP1, EXOSC4, UPF1, DOHH, PTPN23, ZADH2, ARFIP1, STX18, VMP1, TCEA1, ERGIC1, PIAS3, RAD17, EXOSC3, ACOT8, MINA, LRRK1, MOGS, METTL10, CERS4, ATPBD4, ZDHHC6, MAVS, PARL, GNG2, CD200R4, USF1, TYK2, SNAPC1, RBM18, VPS53, ACTR10, and DPCD;
c) a signature comprising, consisting essentially of, or consisting of one or more markers selected from the group consisting of HMGB1, ULBP1, REEP4, HMMR, CMTM7, SIVA1, PAQR4, ATPIF1, NUP85, HSPA2, ENTPD1, HNRNPU, CLIC4, FLOT2, ENOX2, ENPP1, TFRC, HAVCR2, CD2BP2, LGALS1, PGRMC1, USP14, ENTPD5, ATP6AP2, CCL3, IL10RA, ARNT2, KLRE1, CLPTM1, ITGAV, KLRC1, PDIA4, SP1, CCR5, ADAM17, CXCL10, IGSF8, ADAM10, IFNG, TNFRSF9, CD244, CTLA4, ERP44, PSEN1, LY6A, CCRL2, NCOR2, HSP90AA1, IGF2R, PGP, KLRC3, CKLF, TNFRSF1A, TMX3, KLRC2, PDE4D, SMPD1, IDE, SERPINE2, LRPAP1, CSF1, CYSLTR2, LSM1, GRN, IL1F9, LDLR, CD80, GPR174, MIF, MYO9B, ROCK1, GPR56, GPR160, RAC1, PTPN11, CMTM6, ADA, NOTCH2, GPI1, GDI2, P4HB, NRP1, F2R, AGTRAP, PGLYRP1, STX4A, ADAM8, LYST, ITGA1, XPOT, KLRK1, CX3CR1, SEPT2, CCL4, CAST, RPS6KB1, H2-M3, LAG3, CD99L2, PDCD1, ECE1, EZR, NR4A2, SEMA4D, NAMPT, PEAR1, IL12RB1, CD200R4, CD48, LAMP2, IRAK2, CXCR6, GPR65, and GCNT1;
d) a signature comprising, consisting essentially of, or consisting of one or more markers selected from the group consisting of HMMR, SIVA1, ENTPD1, ENPP1, TFRC, CD2BP2, ENTPD5, ITGAV, KLRC1, CCR5, ADAM17, TNFRSF9, CD244, CTLA4, IL3RA, IGF2R, TNFRSF1A, CD80, ADAM8, LAG3, MGL2, CD99L2, SEMA4D, IL12RB1, CD200R4, CD48, and LAMP2;
e) a signature comprising, consisting essentially of, or consisting of one or more markers selected from the group consisting of MXD3, FOXM1, E2F7, RAD54B, E2F8, TCF19, HMGB2, UHRF1, TRIP13, PMF1, BRCA1, BRCA2, HMGB3, MYBL2, HMGB1, PTMA, WHSC1, CHAF1A, RBL1, DNMT1, CCNE1, DEK, E2F2, CHAF1B, EZH2, G2E3, WDHD1, SUZ12, TFDP1, RBBP4, CASP8AP2, RFC1, CDCA4, RBBP8, SSRP1, ANAPC11, TERF1, POLE3, CBFB, CBX3, CTCF, MED30, PBRM1, TFDP2, ITGB3BP, CBX6, NRF1, BAZ1B, E2F3, PIAS1, ILF2, HDAC6, TIMELESS, SMARCA5, MYEF2, TARDBP, EED, HMGXB4, METTL14, E2F4, LIN9, MTF2, MAZ, ATF1, TAF1, TOX, NFYB, HNRNPD, SMARCB1, UHRF2, ASXL1, MED14, NAB1, BRD8, ILF3, MED7, RB1, HDAC3, ERH, TSG101, RNPS1, CCNH, NONO, DEAF1, ZFP91, PKNOX1, L3MBTL2, CDC5L, SP4, KLF11, SMARCC1, HDAC1, GTF2H5, SMARCC2, RUVBL2, ZBTB45, NMRAL1, WIZ, ING1, FOSB, C1D, DICER1, E2F1, THRAP3, RNF4, TSHZ1, SF1, GABPA, GABPB1, SMYD4, CBY1, ARNT2, GABPB2, RFX1, MORF4L2, ZFYVE19, SUB1, HCFC1, TARBP2, GTF3C2, POU2F1, ZNHIT3, TBP, CAND1, PCM1, BAZ1A, ATF6, SREBF2, YAF2, TCERG1, BRPF1, LITAF, SMYD3, RNF5, DPF2, SMARCD2, E2F5, PML, GMEB1, SP1, PSIP1, SP2, CNOT6, OVCA2, PFDN1, COMMD3, ING2, MYNN, HCFC2, AES, LRRFIP2, GTF2E2, YEATS4, CNOT2, MYCBP, PA2G4, TOE1, SMARCD1, NFYC, GMEB2, CEBPB, IKZF5, TFAM, NFIL3, CNOT4, COPS5, GTF2A2, CNOT1, SIN3A, GTF2F1, TSC22D2, ZBTB2, MED4, RUVBL1, AIP, TRIM28, GTF2B, DEDD, CREB1, PRDM1, CTBP1, GTF3C5, TAF10, PSMC3, MED31, RBX1, FUS, PQBP1, ELF2, ATF2, CNOT8, NCOR2, SMARCA4, GNPTAB, TAF6L, CRAMP1L, TCF3, CEBPG, GTF3C3, TAF3, ID2, YBX1, TAF11, YY2, MEN1, PHF6, PHF17, SMARCAD1, RBL2, HTT, HDAC5, ING5, CXXC1, NFKBIL1, CSDA, GTF3A, PRDM10, MECP2, SUDS3, CUX1, ZBTB22, PLRG1, MED24, ETV5, SFMBT1, HLTF, MEF2A, JAZF1, GATAD1, ZBTB11, ZNRD1, RBPJ, XRCC6, GTF2A1, CTNNB1, PURB, CNOT7, ZZEF1, TAF15, TSC22D4, HIRA, ELF4, MED13L, MMS19, MBD1, VHL, VPS72, FOXJ3, UBE2K, SNW1, RASSF7, KEAP1, CAMTA1, MED15, MED8, ING3, CREBZF, TMF1, BOLA2, IKZF2, ARID5B, SND1, PHF20, ZBTB24, SMARCE1, ARID1A, MTA2, KLF10, CCNC, IRF8, ASH2L, MIER3, UIMC1, ELK3, AEBP2, BRF1, SPRY2, RLF, IRF3, NFYA, FLII, BCLAF1, FIZ1, SP3, PARD6A, MTA1, CCDC71, PBX4, PHF5A, NACC1, ATF7IP, HDAC2, GATAD2B, MGA, TRIM27, TCF20, RUFY2, GOLGB1, PYGO2, ZFPL1, HIF1AN, BUD31, TERF2, NOTCH2, UBN1, DNM2, PPIE, HIVEP2, TBC1D2B, COPS2, TAF2, PNRC2, ESRRA, IRF9, SAP30, MIER1, EYA3, NCOA3, LMO4, PREB, NMI, ZBTB40, PCGF1, HMGA1, SARNP, HSBP1, CREM, PTTG1, AGGF1, BMI1, NCOA2, CBX4, TFEB, XAB2, ERF, SAP18, RYBP, MED17, GTF2H3, ZMYND19, SKIL, LZTR1, FOXK2, HTATSF1, TBL1X, PLEKHF2, CIC, TCEA1, CIZ1, TAF9B, TULP4, HMG20B, PIAS3, MED21, TBL1XR1, UTP6, KDM3A, TBX21, ZBTB5, EGR1, ZFYVE27, PHTF1, THOC2, TRAK1, GTF2H1, RNF14, LCORL, IKZF3, HEXIM1, RNF19A, FLI1, MED27, THAP7, MAFF, GATA3, PNN, CBX8, ADNP, NAB2, MED13, NR4A2, PHRF1, SREBF1, STAT2, CHD2, MEF2D, TRIP12, MLX, RLIM, ABT1, KAT5, ETS2, SCAP, RELA, BATF, MED26, KDM2B, KDM6A, CBFA2T2, PIAS4, USF1, ZBTB7A, RNF25, ZBTB1, MED23, ZBTB41, PHF2, KAT2B, KDM2A, CIR1, ZC3H15, ZEB2, REXO4, ZSCAN21, BLZF1, and CENPB; or
f) a signature comprising, consisting essentially of, or consisting of two or more markers each independently selected from any one of the groups as defined in any one of a) to e).

In certain embodiments, the signature further comprises one or more additional markers of activation. In certain embodiments, the one or more additional markers of activation is a co-stimulatory receptor selected from the group consisting of TNFRSF9, TNFRSF4, TNFSF4, TNFRSF18, TNFSF11, TNFRSF13C, CD27, CD28, CD86, ICOS, and TNFSF14.

In certain embodiments, the signature comprises, consists essentially of, or consists of at least one, at least two, or at least three markers selected from the group consisting of ULBP1, HMMR^{high} and REEP4; or the signature comprises, consists essentially of, or consists of ULBP1 and one or both of HMMR^{high} or REEP4.

A further aspect of the invention provides a method for determining whether or not an immune cell has an activated immune phenotype, said method comprising determining in said immune cell the expression of the signature of activation as defined above, whereby expression of the signature indicates that the immune cell has an activated immune phenotype.

Another aspect of the invention relates to a method for determining whether or not a patient would benefit from a therapy aimed at reducing activation of immune cells or a therapy aimed at downregulating of an immune response, the method comprising determining in immune cells from said patient the expression of the signature of activation as defined above, whereby expression of the signature indicates the patient will benefit from the therapy; or for determining whether or not a patient would benefit from a therapy aimed at increasing activation of immune cells or a therapy aimed at upregulating of an immune response, the method comprising determining in immune cells from said patient the expression of the signature of activation as defined above, whereby expression of the signature indicates the patient will likely not benefit from the therapy.

A further aspect of the invention relates to a method for determining the efficacy of a treatment of a patient with a therapy, particularly immune therapy, said method comprising determining in immune cells from said patient the expression of the signature of activation as defined above before and after said treatment and determining the efficacy of said therapy based thereon.

Another aspect of the invention relates to a method for determining the suitability of a compound for modulating an activated immune phenotype and/or modulating an immune response, said method comprising contacting an immune cell expressing the signature of activation as defined above with said compound and determining whether or not said compound can affect the expression of the signature by said cell.

### Modulating T Cell Dysfunction

A systems biology approach was used to show that IL-27 signaling drives the expression of a gene module that includes not only Tim-3, but also Lag-3, TIGIT, and IL-10, all molecules that are associated with T cell dysfunction. The IL-27-induced transcriptional module significantly overlaps with the gene signatures that define dysfunctional T cells in chronic viral infection and cancer, as well as with gene signatures associated with other suppressed or tolerant T cell states. A number of molecules within the IL-27-induced gene module that mediate T cell dysfunction and can be modulated to improve anti-tumor T cell responses in vivo. Finally, using network-based approaches Prdml and c-Maf can be important transcriptional regulators that cooperatively drive the inhibitory gene module. Prdml and c-Maf are also important transcriptional regulators that cooperatively drive the inhibitory gene module. ILT-3 and ILT-3 ligands, such as CD 166, angiopoetins, and angiopoetin-like proteins as important co-stimulatory and co-inhibitory receptors of T cells.

Accordingly, provided herein are methods and compositions for modulating T cell dysfunction by modulating the expression, activity and/or function of at least one target gene or gene product, for example, the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 2 herein, or the pairs of target genes listed herein in Table 1, or any combination thereof.

In one aspect, provided herein is a method of modulating T-cell dysfunction, the method comprising contacting a dysfunctional T-cell with a modulating agent or agents that modulate the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

In one embodiment of this aspect and all other aspects provided herein, the T-cell dysfunction is T-cell exhaustion. In another embodiment of this aspect and all other aspects provided herein, the modulation of T-cell exhaustion comprises a decrease in the exhausted T-cell phenotype, such that functional T-cell activity is increased.

In another embodiment of this aspect and all other aspects provided herein, the modulation of T-cell exhaustion comprises an increase in the exhausted T-cell phenotype, such that functional T-cell activity is decreased.

In another embodiment of this aspect and all other aspects provided herein, the selected target gene or gene product or a combination thereof is/are identified as participating in the inhibition of functional T-cell activity.

In another embodiment of this aspect and all other aspects provided herein, the modulating agent inhibits the expression, activity and/or function of the selected target gene or gene product or combination thereof.

In another embodiment of this aspect and all other aspects provided herein, the selected target gene or combination of target genes is/are identified as participating in the promotion of functional T-cell activity.

In another embodiment of this aspect and all other aspects provided herein, the modulating agent promotes or activates the expression, activity and/or function of the selected target gene or gene product or combination thereof.

In another embodiment of this aspect and all other aspects provided herein, the method further comprises contacting the dysfunctional T-cell with modulating agents that modulate the expression, activity and/or function of at least two target genes or gene products selected from the target genes listed in Table 1 of US Pat. App. Pub. 2019/0255107, Table 1 herein, or any combination thereof.

In another embodiment of this aspect and all other aspects provided herein, the modulating agent comprises a peptide agent, polypeptide agent, a soluble variant of a membrane-associated polypeptide, antibody or antigen-binding fragment thereof agent, a nucleic acid agent, a nucleic acid ligand, or a small molecule agent.

In another embodiment of this aspect and all other aspects provided herein, the methods can further comprise contacting the dysfunctional T-cell with an agent or treatment selected from the group consisting of a PD-1 inhibitor, CTLA4 inhibitor, chemotherapy, radiation therapy, a Braf inhibitor, a MEK inhibitor, a Sting agonist, a TLR agonist, an IDO inhibitor, and an activator or agonist for OX-40, 4-1BB, GITR, CD226, KLRC2, KLRE1, KLRK1, IL12RB1, IL1R1, and/or SLAMF7.

Another aspect provided herein relates to a method of treating a condition involving or characterized by the presence of T cells exhibiting an exhausted or dysfunctional phenotype, the method comprising administering an amount of a modulating agent effective to modulate the expression, activity and/or function of one or more target genes or gene products thereof selected from the target genes listed in Table 1 of US Pat. App. Pub. 2019/0255107, Table 1 herein, or any combination thereof.

In one embodiment of this aspect and all other aspects provided herein, the condition is cancer or a persistent infection.

Provided herein in another aspect is a pharmaceutical composition for modulating T cell dysfunction, the composition comprising a first modulating agent and a second modulating agent that modulate the expression, activity and/or function of two or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein or any combination thereof.

Another aspect provided herein relates to a pharmaceutical composition for modulating T cell dysfunction, the composition comprising a first modulating agent that inhibits the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof and a second modulating agent that promotes the expression, activity and/or function of one or more target genes or gene products thereof.

Also provided herein, in another aspect, is a pharmaceutical composition for modulating T cell dysfunction, the composition comprising a modulating agent that modulates the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof and an agent selected from the group consisting of a PD-1 inhibitor, a CTLA4 inhibitor, chemotherapy, a Braf inhibitor, a MEK inhibitor, a Sting agonist, a TLR agonist, an IDO inhibitor, and an agonist for OX-40, 4-IBB, GITR, CD226, KLRC2, KLRE1, KLRK1 IL12RB 1, IL1R, and SLAMF7.

Also provided herein, in another aspect, are pharmaceutical compositions for modulating T cell dysfunction, the composition comprising at least one modulating agent that modulates the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof. In another aspect, the pharmaceutical compositions comprise at least two modulating agents that modulate the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

Also provided herein, in another aspect, are pharmaceutical compositions for modulating T cell dysfunction, the composition comprising at least one modulating agent that modulates the expression, activity and/or function of one or more target genes or gene products thereof selected from the target genes listed in Table 5 of US Pat. App. Pub. 2019/0255107, Table 6 of US Pat. App. Pub. 2019/0255107, Table 7 of US Pat. App. Pub. 2019/0255107, Table 8 of US Pat. App. Pub. 2019/0255107, Table 9 of US Pat. App. Pub. 2019/0255107, or any combination thereof. In another aspect, the pharmaceutical compositions comprise at least two modulating agents that modulate the expression, activity and/or function of one or more target genes or gene products thereof selected from the target genes listed in Table 5 of US Pat. App. Pub. 2019/0255107, Table 6 of US Pat. App. Pub. 2019/0255107, Table 7 of US Pat. App. Pub. 2019/0255107, Table 8 of US Pat. App. Pub. 2019/0255107, Table 9 of US Pat. App. Pub. 2019/0255107, or any combination thereof.

Another aspect provided herein relates to a pharmaceutical composition for modulating T cell dysfunction, the composition comprising an inhibitor of the expression and/or activity of PDPN, an inhibitor of the expression and/or activity of PROCR, or a combination thereof.

Also provided herein in another aspect is a pharmaceutical composition for modulating T cell dysfunction comprising: (a) an inhibitor of the expression and/or activity of PDPN and an inhibitor of the expression and/or activity of PROCR; and (b) an inhibitor of the expression and/or activity of at least one of the molecules selected from the group consisting of TIGIT, LAG3, LILRB4, and KLRC1; and/or an activator of the expression and/or activity of at least one of the molecules selected from the group consisting of CD226, OX-40, GITR, TNFSF9 (4-1BB), KLRC2, KLRE1, KLRK1, IL12RB1, IL1R, and SLAMF7.

Provided herein in another aspect is a pharmaceutical composition for modulating an IL-27-regulated co-inhibitory module comprising: (a) an inhibitor of the expression and/or activity of at least one of the molecules selected from the group consisting of PDPN, PROCR, TIGIT, LAG3, LILRB4, ALCAM, and KLRC1; and (b) an activator of the expression and/or activity of at least one of the molecules selected from the group consisting of CD226, OX-40, GITR, TNFSF9 (4-1BB), KLRC2, KLRE1, KLRK1, IL12RB1, IL1R1, and SLAMF7.

In one embodiment of this aspect and all other aspects provided herein, the composition further comprises an inhibitor of the expression and/or activity of TIM-3, of PD-1, of CTLA4, or any combinations thereof (TIM-3 and PD-1; PD-1 and CTLA4; TIM-3 and CTLA4; or TIM-3, PD-1, and CTLA4).

In another embodiment of this aspect and all other aspects provided herein, the inhibitors and activators are selected from an antibody or antigen binding fragment thereof, a small molecule compound, a protein or peptide molecule, a DNA or RNA aptamer, an antisense or siRNA molecule, and a structural analog.

In another embodiment of this aspect and all other aspects provided herein, the inhibitors and activators are selected from an antibody or antigen binding fragment thereof, a small molecule compound, a protein or peptide molecule, a DNA or RNA aptamer, an antisense or siRNA molecule, and a structural analog.

In another embodiment of this aspect and all other aspects provided herein, the antibody or antigen binding fragment thereof, a small molecule compound, a protein or peptide molecule, a DNA or RNA aptamer, an antisense or siRNA molecule, and a structural analog is selected from: an anti-CTLA4 antibody, an anti-PD-1 antibody, or a PDL-1 antagonist. In certain embodiments, the antibody or antigen binding fragment thereof is selected from the group consisting of: nivolumab, pembrolizumab, lambrolizumab, ipilimumab, and atezolizumab.

Another aspect provided herein relates to a method of modulating an IL-27-regulated co-inhibitory module in a subject in need thereof, the method comprising administering a pharmaceutical composition comprising an inhibitor of the expression and/or activity of PDPN, an inhibitor of the expression and/or activity of PROCR, or a combination thereof.

An additional aspect provided herein relates to a method of modulating an IL-27-regulated co-inhibitory module in a subject in need thereof, the method comprising: (a) administering a pharmaceutical composition comprising an inhibitor of the expression and/or activity of PDPN, and an inhibitor of the expression and/or activity of PROCR; and (b) administering a pharmaceutical composition comprising an inhibitor of the expression and/or activity of at least one of the molecules selected from the group consisting of an inhibitor of the expression and/or activity of TIGIT, LAG3, LILRB4, and KLRC1; and/or an activator of the expression and/or activity of at least one of the molecules selected from the group consisting of CD226, OX-40, GITR, TNFSF9 (4-1BB), KLRC2, KLRE1, KLRK1, IL12RB1, IL1R1, and SLAMF7.

Also provided herein in another aspect is a method of modulating an IL-27-regulated co-inhibitory module in a subject in need thereof, the method comprising: (a) administering a pharmaceutical composition comprising an inhibitor of the expression and/or activity of at least one of the molecules selected from the group consisting of PDPN, PROCR, TIGIT, LAG3, LILRB4, ALCAM and KLRC1; and (b) administering a pharmaceutical composition comprising an activator the expression and/or activity of at least one of the molecules selected from the group consisting of CD226, OX-40, GITR, TNFSF9 (4-1BB), KLRC2, KLRE1, KLRK1, IL12RB1, IL1R1, and SLAMF7.

Also provided herein in another aspect is a method of treating a condition involving or characterized by the presence of T cells exhibiting an exhausted phenotype, the method comprising administering an amount of a modulating agent effective to modulate the expression, activity and/or function of one or more target genes or gene products thereof selected from the group consisting of: the subset of genes listed in Table 5 of US Pat. App. Pub. 2019/0255107, the subset of genes listed in Table 6 of US Pat. App. Pub. 2019/0255107, the subset of genes listed in Table 7 of US Pat. App. Pub. 2019/0255107, the subset of genes listed in Table 8 of US Pat. App. Pub. 2019/0255107, and the subset of genes listed in Table 9 of US Pat. App. Pub. 2019/0255107.

In some aspects, provided herein are methods of treating a disease or disorder characterized by aberrant or unwanted T-cell functional activity in a subject in need thereof, the method comprising administering a therapeutically effective amount of a modulating agent effective to modulate the expression, activity and/or function of one or more target genes or gene products thereof selected from the target genes listed in Table 1 of US Pat. App. Pub. 2019/0255107, Table 1 herein, or any combination thereof.

In one aspect, provided herein is a method of modulating T cell dysfunction, the method comprising contacting a dysfunctional T cell with a modulating agent or agents that modulate the expression, activity and/or function of ILT-3.

In another embodiment of this aspect the modulating agent promotes the expression, activity and/or function of the ILT-3 gene or gene product or combination thereof.

In another embodiment of this aspect the modulating agent inhibits the expression, activity and/or function of the ILT-3 gene or gene product or combination thereof.

In another embodiment of this aspect the modulating agent inhibits binding of ILT-3 to one or more ILT-3 ligands.

In another embodiment of this aspect the one or more ILT-3 ligands is selected from integrin αvβ3, CD 166, ANGPT1, ANGPT2, ANGPT3, ANGPT4, ANGPTL1, ANGPTL2, ANGPTL3, ANGPTL4, ANGPTL5, ANGPTL6, ANGPTL7, and ANGPTL8.

In another embodiment of this aspect the modulating agent comprises a peptide agent, polypeptide agent, a soluble variant of a membrane-associated polypeptide, antibody agent, a nucleic acid agent, a nucleic acid ligand, a nuclease agent, or a small molecule agent.

In one aspect, provided herein is a method of treating a condition involving or characterized by the presence of T cells exhibiting an exhausted phenotype, the method comprising administering an amount of a modulating agent effective to modulate the expression, activity and/or function of ILT-3 to a subject in need thereof.

In one aspect, provided herein is a method of determining the presence of T cells exhibiting an exhausted phenotype, the method comprising detecting, in a sample comprising T cells, a level of expression, activity and/or function of ILT-3, and comparing the detected level to a reference, wherein a difference in the detected level relative to the reference indicates the presence of T cells exhibiting an exhausted phenotype.

In one aspect, provided herein is a method of modulating T cell dysfunction, the method comprising contacting a dysfunctional T cell with a modulating agent or agents that modulate the expression, activity and/or function of an angiopoetin or angiopoietin-like protein.

In another embodiment of this aspect the modulating agent promotes or inhibits the expression, activity and/or function of one or more genes selected from ANGPT1, ANGPT2, ANGPT3, ANGPT4, ANGPTL1, ANGPTL2, ANGPTL3, ANGPTL4, ANGPTL5, ANGPTL6, ANGPTL7, and ANGPTL8 or gene products thereof or combinations thereof.

In one aspect, provided herein is a method of treating a condition involving or characterized by the presence of T cells exhibiting an exhausted phenotype, the method comprising administering an amount of a modulating agent effective to modulate the expression, activity and/or function of an angiopoetin or angiopoietin-like protein to a subject in need thereof.

In one aspect, provided herein is a method of determining the presence of T cells exhibiting an exhausted phenotype, the method comprising detecting, in a sample comprising T cells, a level of expression, activity and/or function of an angiopoetin or angiopoietin-like protein, and comparing the detected level to a reference, wherein a difference in the detected level relative to the reference indicates the presence of T cells exhibiting an exhausted phenotype.

In one aspect, provided herein is a method of modulating T cell dysfunction, the method comprising contacting a dysfunctional T cell with a modulating agent or agents that modulate the expression, activity and/or function of CD 166. In another embodiment of this aspect the modulating agent promotes or inhibits the expression, activity and/or function of the CD 166 gene or gene product or combination thereof.

In one aspect, provided herein is a method of treating a condition involving or characterized by the presence of T cells exhibiting an exhausted phenotype, the method comprising administering an amount of a modulating agent effective to modulate the expression, activity and/or function CD 166 to a subject in need thereof.

In one aspect, provided herein is a method of determining the presence of T cells exhibiting an exhausted phenotype, the method comprising detecting, in a sample comprising T cells, a level of expression, activity and/or function of CD 166, and comparing the detected level to a reference, wherein a difference in the detected level relative to the reference indicates the presence of T cells exhibiting an exhausted phenotype.

In one aspect, provided herein is a method of modulating T-cell dysfunction, the method comprising contacting a dysfunctional T-cell with a modulating agent or agents that modulate the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

Another aspect provided herein relates to a pharmaceutical composition for modulating T cell dysfunction, the composition comprising a first modulating agent that inhibits the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

or any combination thereof and a second modulating agent that promotes the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

Another aspect provided herein relates to a pharmaceutical composition for modulating T cell dysfunction, the composition comprising a first modulating agent that inhibits the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof and a second modulating agent that promotes the expression, activity and/or function of one or more target genes or gene products thereof selected from the target genes listed in Table 1 of US Pat. App. Pub. 2019/0255107, Table 10 of US Pat. App. Pub. 2019/0255107, Table 12 of US Pat. App. Pub. 2019/0255107, Table 13 of US Pat. App. Pub. 2019/0255107, Table 1 herein, Table 2 herein, or any combination thereof.

In another embodiment of this aspect and all other aspects provided herein, the composition further comprises an inhibitor of the expression and/or activity of TIM-3 and an inhibitor of the expression and/or activity of PD-1. In another embodiment of this aspect and all other aspects provided herein, the composition further comprises an inhibitor of the expression and/or activity of TIM-3 and an inhibitor of the expression and/or activity of CTLA4. In another embodiment of this aspect and all other aspects provided herein, the composition further comprises an inhibitor of the expression and/or activity of CTLA4 and an inhibitor of the expression and/or activity of PD-1. In another embodiment of this aspect and all other aspects provided herein, the composition further comprises an inhibitor of the expression and/or activity of CTLA4, and an inhibitor of the expression and/or activity of PD-1 and an inhibitor of the expression and/or activity of TEVI-3.

In another aspect, the present invention provides for a method for generating the modified immune cell of any embodiment described herein, the method comprising (i) providing an isolated immune cell, and (ii) modifying said isolated immune cell such as to comprise an agent capable of inducibly altering expression or activity of PDPN, PROCR, or PRDM1 and c-MAF.

### T-Cells Having Altered FAS-STAT1 Binding

Fas can promote the generation and stability of Th17 cells and prevented their differentiation into Th1 cells. In one aspect, the present invention provides for an isolated T cell modified to comprise altered FAS-STAT1 binding.

In certain embodiments, the T cell is modified to express a recombinant polypeptide capable of antagonizing FAS-STAT1 interaction. In certain embodiments, the polypeptide does not affect the binding of FAS to FAS-L. In certain embodiments, the polypeptide does not affect the binding of FAS to FADD.

In certain embodiments, the T cell is modified to express a recombinant polypeptide that is capable of adopting a FAS ligand bound conformation, is inactivated for apoptotic signaling, and is able to bind to STAT1. Thus, the recombinant polypeptide is only able to antagonize FAS-STAT1 binding. In certain embodiments, the polypeptide does not affect the binding of FAS to FAS-L. In certain embodiments, the polypeptide does not affect the binding of FAS to FADD.

In certain embodiments, the T cell is modified to over-express STAT1. Thus, in certain embodiments, increased expression of STAT1 can saturate binding to FAS and shift T cell balance towards a Th1 phenotype.

In certain embodiments, the T cell is modified to abolish or knockdown expression or activity of STAT1 and is differentiated under Th17 conditions. The Th17 conditions may comprise cultures supplemented with IL-6 and TGF-β1 or supplemented with IL-1β, IL-6 and IL-23. The T cell may comprise a genetic modifying agent targeting STAT1. The genetic modifying agent may comprise a CRISPR system, a zinc finger nuclease system, a TALEN, or a meganuclease. The CRISPR system may comprise Cas9 or Cpf1 and target the STAT1 gene. The CRISPR system may comprise a Cas13 system and target STAT1 mRNA. The Cas13 system may comprise Cas13-ADAR.

In certain embodiments, the T cell is modified to comprise a non-silent mutation in FAS and/or STAT1, wherein the mutation inhibits FAS-STAT1 binding. The mutation may alter a post-translational modification site in FAS and/or STAT1 that alters FAS-STAT1 binding. The mutation may not inhibit FAS apoptotic signaling. The T cell may comprise a genetic modifying agent targeting FAS and/or STAT1. The genetic modifying agent may comprise a CRISPR system, a zinc finger nuclease system, a TALEN, or a meganuclease. The CRISPR system may comprise a Cas13 system and target FAS and/or STAT1 mRNA. The Cas13 system may comprise Cas13-ADAR.

In certain embodiments, the T cell is modified to decrease, but not eliminate expression or activity of FAS. The T cell may be differentiated under Th17 conditions. The Th17 conditions may comprise cultures supplemented with IL-6 and TGF-β1 or supplemented with IL-1β, IL-6 and IL-23. The T cell may comprise a genetic modifying agent targeting FAS. The genetic modifying agent may comprise a CRISPR system, a zinc finger nuclease system, a TALEN, or a meganuclease. The CRISPR system may comprise a Cas13 system and target FAS mRNA. The Cas13 system may comprise Cas13-ADAR.

In certain embodiments, the isolated T cell of any embodiment is a Th17 cell. In certain embodiments, the T cell is a naïve Th0 cell. In certain embodiments, the T cell is a tumor infiltrating lymphocyte (TIL). In certain embodiments, the T cell expresses an endogenous T cell receptor (TCR) or chimeric antigen receptor (CAR) specific for a tumor antigen. In certain embodiments, the T cell is expanded. In certain embodiments, the T cell is modified to express a suicide gene, wherein the modified T cell can be eliminated upon administration of a drug.

In another aspect, the present invention provides for a pharmaceutical composition comprising the isolated T cell as described in any of the paragraphs above.

In another aspect, the present invention provides for a method of treating cancer comprising administering the pharmaceutical composition as described in the above paragraphs to a subject in need thereof, whereby a Th17 response is enhanced.

In another aspect, the present invention provides for a method of treating cancer comprising administering the pharmaceutical composition as described in the above paragraphs to a subject in need thereof, whereby a Th1 response is enhanced.

In another aspect, the present invention provides for a method of treating an inflammatory or autoimmune disease comprising administering the pharmaceutical composition as described in the above paragraphs to a subject in need thereof.

In another aspect, the present invention provides for a method of modulating T cell balance, the method comprising perturbing FAS-STAT1 binding in a T cell or a population of T cells. In certain embodiments, perturbing comprises introducing a genetic modifying agent targeting FAS and/or STAT1 to the T cell or population of T cells. The genetic modifying agent may comprise a CRISPR system, a zinc finger nuclease system, a TALEN, or a meganuclease. The CRISPR system may comprise a Cas13 system and target FAS and/or STAT1 mRNA. The Cas13 system may comprise Cas13-ADAR.

In certain embodiments, the T cell or population of T cells may be modified to comprise a non-silent mutation in FAS and/or STAT1, wherein the mutation inhibits FAS-STAT1 binding. The mutation may alter a post-translational modification site in FAS and/or STAT1. In certain embodiments, T cell differentiation is shifted towards Th1 cells and/or is shifted away from Th17 cells.

In certain embodiments, the T cell or population of T cells is modified to comprise a decrease or knockout in expression of STAT1. In certain embodiments, T cell differentiation is shifted towards Th17 cells and/or is shifted away from Th1 cells.

In certain embodiments, the T cell or population of T cells is modified to comprise a decrease in expression of FAS. In certain embodiments, FAS mRNA is targeted and the decrease is temporary. In certain embodiments, T cell differentiation is shifted towards Th1 cells and/or is shifted away from Th17 cells.

In certain embodiments, the CRISPR system is administered as a ribonucleoprotein (RNP) complex.

In certain embodiments, modulating T cell balance comprises contacting the T cell or population of T cells with an inhibitor of FAS-STAT1 binding. In certain embodiments, modulating T cell balance comprises increasing expression of STAT1 in the T cell or population of T cells. In certain embodiments, T cell differentiation is shifted towards Th1 cells and/or is shifted away from Th17 cells.

In certain embodiments, the T cell or population of T cells comprise naïve Th0 T cells. The cells may be cultured under Th1 or Th17 conditions. The Th17 conditions may comprise cultures supplemented with IL-6 and TGF-β1 or supplemented with IL-1β, IL-6 and IL-23. In certain embodiments, FAS is bound by FAS ligand.

In another aspect, the present invention provides for a method of modulating an immune response in a subject in need thereof, the method comprising administering to the subject a therapeutically effective amount of an inhibitor of FAS-STAT1 binding. The method may be for treating an aberrant immune response in said subject. The method may be for treating an autoimmune disease. The autoimmune disease may be selected from Multiple Sclerosis (MS), Irritable Bowel Disease (IBD), Crohn's disease, spondyloarthritides, Systemic Lupus Erythematosus (SLE), Vitiligo, rheumatoid arthritis, psoriasis, Sjögren's syndrome, and diabetes. The method may be for treating an inflammatory disorder. The inflammatory disorder may be selected from psoriasis, inflammatory bowel diseases (IBD), allergic asthma, food allergies and rheumatoid arthritis.

In certain embodiments, the inhibitor of FAS-STAT1 binding does not affect the binding of FAS to FAS-L. In certain embodiments, the inhibitor of FAS-STAT1 binding does not affect the binding of FAS to FADD. In certain embodiments, the inhibitor binds to the cytoplasmic domain of FAS. In certain embodiments, the inhibitor does not bind to the extracellular domain of FAS.

In certain embodiments, the inhibitor is an antibody, antibody fragment, intrabody, antibody-like protein scaffold, polypeptide, genetic modifying agent, or small molecule. The genetic modifying agent may comprise a CRISPR system, a zinc finger nuclease system, a TALEN, or a meganuclease.

In another aspect, the present invention provides for a pharmaceutical composition comprising an inhibitor of FAS-STAT1 binding. The inhibitor of FAS-STAT1 binding may not affect the binding of FAS to FAS-L. The inhibitor of FAS-STAT1 binding may not affect the binding of FAS to FADD. The inhibitor may bind to the cytoplasmic domain of FAS. The inhibitor may not bind to the extracellular domain of FAS. In certain embodiments, the inhibitor is an antibody, antibody fragment, intrabody, antibody-like protein scaffold, polypeptide, genetic modifying agent, or small molecule.

In certain embodiments, modulating T cell balance comprises providing the T cell or population of T cells with a FAS polypeptide, wherein said polypeptide is able to bind to STAT1. The polypeptide may adopt a FAS ligand bound conformation and may be inactivated for apoptotic signaling. In certain embodiments, T cell differentiation is shifted towards Th17 cells and/or is shifted away from Th1 cells. In certain embodiments, providing a FAS polypeptide comprises providing a nucleic acid encoding the polypeptide. The nucleic acid may be provided as a vector. The polypeptide may be a membrane bound polypeptide. The polypeptide may not bind to FAS-L. The polypeptide may not comprise the extracellular domain of FAS. Binding of the polypeptide to STAT1 may not lead to phosphorylation of STAT1. Binding of the polypeptide to STAT1 may prevent or reduce nuclear translocation of STAT1.

In another aspect, the present invention provides for a method of treating cancer or an infectious disease in a subject in need thereof comprising: isolating Th17 cells from the blood of the subject; transforming the isolated Th17 cells with one or more vectors encoding: (i) a CAR or endogenous TCR directed against a tumor antigen or an infectious disease antigen, and (ii) a CRISPR system targeting STAT1; and administering the transformed Th17 cells to the subject.

In another aspect, the present invention provides for a method of treating autoimmunity in a subject in need thereof comprising: isolating Th17 cells from the blood of the subject; transforming the isolated Th17 cells with one or more vectors encoding a CRISPR system targeting FAS; and administering the Th17 FAS mutant cells to the subject.

### CPB Therapy Responder and Non-Responder Gene Signatures

In some embodiments, the T cells (e.g. CAR T cells described herein) can be a checkpoint blockade (CPB) therapy responder or a non-responder cell. Responder and on-responder cells can be identified by detecting a CBP therapy responder gene signature or a CBP therapy non-responder gene signature in a T cell (e.g. CAR T cell) described herein. In some embodiments, responder cells can have improved efficacy over non-responder cells when administered with a checkpoint blockade therapy. In some embodiments, responder cells can have reduced dysfunction and/or exhaustion when administered with a checkpoint blockade therapy.

In one aspect, the present invention provides for a method of detecting a checkpoint blockade (CPB) therapy responder gene signature comprising, detecting in CD45+ cells obtained from a biological sample the expression of a gene signature comprising one or more genes or polypeptides selected from the group consisting of: TCF7; or TCF7, PLAC8, LTB, and CCR7; or TCF7, LEF1, S1PR1, PLAC8, LTB, CCR7, IGHD, PAX5, FCRL1, FCER2, CD19, CD22, BANK1, MS4A1, BLK, RALGPS2 and FAM129C; or TCF7, PLAC8, LTB, LY9, SELL, IGKC and CCR7.

In another aspect, the present invention provides for a method of detecting a checkpoint blockade (CPB) therapy responder gene signature comprising, detecting in CD8+ T cells obtained from a biological sample the expression of a gene signature comprising one or more genes or polypeptides selected from the group consisting of: TCF7; or TCF7 and IL7R; or TCF7, IL7R, FOSL2, REL, FOXP1, and STAT4; or TCF7, PLAC8, LTB, and CCR7; or TCF7, LEF1, S1PR1, PLAC8, LTB, and CCR7; or TCF7, IL7R, GPR183, and MGAT4A; or TCF7, IL7R, GPR183, LMNA, NR4A3, CD55, AIM1, MGAT4A, PER1, FOSL2, TSPYL2, REL, FAM177A1, YPEL5, TC2N and CSRNP1; or TCF7, IL7R, GPR183, LMNA, NR4A3, CD55, AIM1, MGAT4A, PER1, FOSL2, TSPYL2, REL, FAM177A1, YPEL5, TC2N, CSRNP1, FAM65B, PIK3R1, RGPD6, SKIL, TSC22D2, USP36, FOXP1, EGR1, MYADM, ZFP36L2, FAM102A, RGCC, PDE4B, PFKFB3, FOSB, DCTN6 and BTG2; or CD8_G genes listed in Table 6 of US Pat. App. Pub. 2019/0255107.

In certain embodiments, the CD8 T cells having a responder signature does not express ENTPD1 (CD39) and HAVCR2.

In another aspect, the present invention provides for a method of detecting a checkpoint blockade (CPB) therapy non-responder gene signature comprising, detecting in CD45+ cells obtained from a biological sample the expression of a gene signature comprising one or more genes or polypeptides selected from the group consisting of: ENTPD1 and HAVCR2; or CCL3, CD38 and HAVCR2; or CD38, PDCD1, CCL3, SNAP47, VCAM1, HAVCR2, FASLG, ENTPD1, SIRPG, MYO7A, FABP5, NDUFB3, UBE2F, CLTA and SNRPD1; or FASLG, VCAM1, CCL3, LAG3, CXCR6, IFNG, PDCD1, KLRD1, HAVCR2, SIRPG, SNAP47, DTHD1, PRF1, GZMH, F2R, CD38, CXCL13, TNFRSF4, TNFRSF18, MAF, ETV7, CD4, CTLA4, FCRL6, SPON2, KLRG1, TRGC1, A2M, FCGR3A, GZMA, HOPX, NKG7, PXN, TNFRSF9, GEM, NAB1, DFNB31, CADM1, CRTAM, GPR56, MYO7A, DUSP4, METRNL and PHLDA1; or LAYN, GEM, VCAM1, RDH10, TNFRSF18, FAM3C, AFAP1L2, KIR2DL4, MTSS1, ETV1, CTLA4, MYO7A, ENTPD1, TNFRSF9, CADM1, DFNB31, CXCL13, HAVCR2, GPR56, GOLIM4, NAB1, PHLDA1, TGIF1, SEC14L1, IGFLR1, NAMPTL, PAM, HSPB1, TNIP3, BPGM, TP53INP1, TRPS1, UBE2F, NDFIP2, PON2, PELI1, METRNL, SNAP47 and APLP2; or CCL3, LGALS1, CD38, EPSTI1, WARS, PLEK, HAVCR2, LGALS3, FABP5, MT2A, GBP1, PLSCR1, CCR5, GSTO1, ANXA5, GLUL, PYCARD, TYMP, IFI6, VAMP5, OASL, GZMB, TXN, SQRDL, RHOC, AP2S1, GZMH, CCL4L2, SNAP47, LAP3, ATP6V1B2, CCL4L1, LAMP2, PSMA4, SERPINB1, HIGD1A, UBE2F, TALDO1, CD63, CLTA, S100A11, PHPT1, GBP4, PRDX3, PSMB2, BST2, GBP5, CTSC, NDUFB3, NPC2, GALM, GLIPR2, CCL4, PRF1, IFNG, IFI30, CHST12, ISG15, MYD88, IDH2, MTHFD2, CHMP2A, NDUFA9, CHMP5, CALM3, ANXA2, PPT1, GTF3C6, NDUFAB1, CXCR6, RNF181, LGALS9, COX5A, OAS2, PDCD1, SNRPC, BHLHE40, TWF2, SLAMF7, TXN2, CARD16, ANAPC11, MRPL51, LIMS1, NDUFA12, RANBP1, GBP2, PSMC1, ACTR1A, CD2BP2, VDAC1, EMC7, MX1, GPS1, ATP5J2, USMG5, SHFM1, ATP5I, FAM96A, CASP1, PARP9, NOP10, GNG5, CYC1, RAB11A, PGAM1, ENTPD1, PDIA6, PSMC3, TMBIM1, UBE2L6, PSMA6, EIF6, DCTN3, SEC11A, CSTB, ETFB, DBI, GRN, ELOVL1, UBE2L3, PSMB3, NDUFB7, DOK2, SEC61G, IGFLR1, ATP5H, COPZ1, ATP6V1F, BNIP3L, NUTF2, AKR1A1, MDH2, VAMP8, ROMO1, CXCR3, SAMHD1, NUCB1, ACTN4, ZYX, FLOT1, BLOC1S1, STAT1, VIMP, PAM, NUDT21, MYO1G, C17orf49, GTF2A2, HIST2H2AA4, C19orf10, ABI3, TRAPPC5, PSMC4, NDUFC2, HN1, SNRPD3, CMC1, RAB27A, NDUFA6, POMP, PFKP, ATP5G3, TMEM179B, PSMD9, IRF7, CNIH1, DYNLRB1, APOL2, TKT, DCTN2, GSDMD, STOM, CTSD, KDELR2, ATP5J, RPS27L, PSME2, DRAP1, NDUFB10, DECR1, GSTP1, TMED9, MGAT1, HSPB1, COX8A, ZEB2, ILK, PSMB6, HK1, CD58, TMX1, GZMA, SRI, PSMG2, ARL8B, NKG7, GPX1, ACP5, CHP1, GPR171, ATP6V0B, KLRD1, H2AFY, PPM1G, PRDX5, PSMA5, FBXW5, ATP6AP1, CD4, SNRPD1, XAF1, LY6E, DYNLT1, AK2, PSMA2, YIPF3, S100A10, SCP2, MRPS34, PSMD4, CDC123, BTG3, TMEM258, TSPO, SDHB, TCEB1, WDR83OS, HCST, NAA10, CTSB, YARS, GLRX, RBCK1, RBX1, LAMTOR1, UQCRFS1, NDUFB4, CAPZA2, BRK1, ADRM1, NDUFB2, ETFA, VDAC3, NUDT5, IFITM3, BANF1, ZNHIT1, CAPG, NHP2, LASP1, TOMM5, MVP, CTSW, AURKAIP1, RARRES3, PSMB10, TMEM173, SLX1A, APOBEC3G, GIMAP4, EIF4E, CTLA4, NDUFS8, CYB5B, PIK3R5, HEXB, STXBP2, PSMD8, SEC61B, RGS10, PHB, ATP5C1, ARF5, SUMO3, PRDX6, RNH1, ATP5F1, UQCRC1, SARNP, PLIN2, PIN1, SDHC, SF3B14, CAPRIN1, POLR2G, COX7B, UQCR10, FBXO7, NDUFB6, S100A4, PRELID1, TRPV2, SF3B5, MYO1F, SCAMP2, RNF7, CXCL13, RAB1B, SHKBP1, PET100, HM13, VTI1B, S100A6, ARPC5, FDPS, MINOS1, RAB10, NEDD8, BATF, PHB2, ERH, NCOA4, PDIA4, PSMB9, C11 orf48, TMEM50A, TIGIT, NDUFA11, NELFE, COX6C, SLA2, PSMB8, NDUFS7, RER1, RAB8A, CAPN1, MRPL20, COX5B, SEC13, FKBP1A, PRDM1, RAB1A, RHOG, CYB5R3, AIP, ABRACL, PSMB7, COX6B1, PSMD7, PPA1, PCMT1, SURF4, ENY2, TCEB2, MAP2K3, AL353354.2, AKIRIN2, MAPRE1, GRSF1, DUSP4, ATG3, SRGAP2, ATP6V0D1, NELFCD, LRPAP1, C14orf166, SNRPB2, CHMP4A, SFT2D1, CASP4, NME1-NME2, FAM96B, FDFT1, SLC25A39, LMAN2, MDH1, RHBDD2, ARPC5L, TBCA, EBP, SEC14L1, EIF2S2, CST7, STARD7, SOD2, SPN, FAM32A, SEC11C, TNFRSF1B, POLR2E, NDUFA13, OSTC, UFC1, C18orf32, SRP19, C14orf2, UQCR11, PDCD6, AP2M1, PPP1CA, ATP6AP2, SSR3, UNC13D, FERMT3, ARHGAP1, EIF3I, CECR1, MRPS6, DNPH1, DCXR, PSMF1, SNRPG, CNDP2, ANXA11, SLMO2, C16orf13, CAPN2, BSG, LAMTOR5, SIVA1, TRAPPC1, TMCO1, PSMD13, PSMB1, RSU1, NDUFA1, TUBB, DCTN1, SH3GLB1, BCAP31, RTFDC1, UFD1L, GPI, DNAJB11, SNX17, SH2D2A, Clorf43, BUD31, PSTPIP1, CTSA, TPST2, MPV17, APMAP, CMC2, UQCRQ, TBCB, C9orf16, PARK7, ATP5EP2, SHISA5, SMC4, TAP1, SCAND1, SIRPG, HDLBP, EMC4, FIS1, TPI1, GOLGA7, POLR2J, EIF2S1, UBA3, P4HB, UQCRH, CSNK2B, SZRD1, NDUFA3, ATP5O, DERL2, COPS6, COPE, SNX6, FLII and ERGIC3.

In another aspect, the present invention provides for a method of detecting a checkpoint blockade (CPB) therapy non-responder gene signature comprising, detecting in CD8+ T cells obtained from a biological sample the expression of a gene signature comprising one or more genes or polypeptides selected from the group consisting of: ENTPD1 and HAVCR2; or CCL3, CD38 and HAVCR2; or CD38, CCL3, VCAM1, GOLIM4, HAVCR2, PRDX3, ENTPD1, PTTG1, CCR5, TRAFD1, PDCD1, CXCR6, BATF, PTPN6, LAG3 and CTLA4; or LAYN, GEM, VCAM1, RDH10, TNFRSF18, FAM3C, AFAP1L2, KIR2DL4, MTSS1, ETV1, CTLA4, MYO7A, ENTPD1, TNFRSF9, CADM1, DFNB31, CXCL13, HAVCR2, GPR56, GOLIM4, NAB1, PHLDA1, TGIF1, SEC14L1, IGFLR1, NAMPTL, PAM, HSPB1, TNIP3, BPGM, TP53INP1, TRPS1, UBE2F, NDFIP2, PON2, PELI1, METRNL, SNAP47 and APLP2; or CD38, EPSTI1, GOLIM4, WARS, PDCD1, CCL3, SNAP47, VCAM1, SKA2, HAVCR2, LGALS9, PRDX3, FASLG, ENTPD1, FABP5, SIRPG, LSM2, NDUFB3, TRAFD1, UBE2F, NMI, IFI35, CLTA, MTHFD1, MYO7A, IFI27L2, MCM5, STMN1, ID3, RGS3, SNRPD1, PTTG1 and FIBP; or CD8_B genes listed in Table 6 of US Pat. App. Pub. 2019/0255107.

In certain embodiments, the biological sample is a tumor sample obtained from a subject. In certain embodiments, the gene signature is detected in tumor infiltrating lymphocytes (TILs). In certain embodiments, the biological sample comprises ex vivo or in vitro immune cells, preferably CD8+ T cells. In certain embodiments, the gene signature is detected by deconvolution of bulk expression data such that gene expression in immune cells is detected.

In certain embodiments, detecting a higher proportion immune cells expressing a responder signature as compared to a non-responder signature indicates sensitivity to checkpoint blockade (CPB) therapy and an increased overall survival, and wherein detecting a higher proportion immune cells expressing a non-responder signature indicates resistance to checkpoint blockade (CPB) therapy and a decreased overall survival. In certain embodiments, detecting a higher proportion of TCF7+CD8+ as compared to TCF7-CD8+ T cells indicates sensitivity to checkpoint blockade (CPB) therapy and an increased overall survival, and wherein detecting a higher proportion TCF7-CD8+ as compared to TCF7+CD8+ T cells indicates resistance to checkpoint blockade (CPB) therapy and a decreased overall survival. In certain embodiments, TCF7+CD8+ and TCF7-CD8+ T cells are detected by immunofluorescence. In certain embodiments, the checkpoint blockade (CPB) therapy comprises anti-CTLA4, anti-PD-L1, anti-PD1 therapy or combinations thereof.

In another aspect, the present invention provides for a method of predicting cancer clinical outcome in a subject in need thereof comprising detecting in a sample obtained from the subject the ratio of immune cells enriched for expression of a responder gene signature as compared to immune cells enriched for expression of a non-responder gene signature, wherein a ratio greater than one indicates sensitivity to an immunotherapy and an increased overall survival, and wherein a ratio less than one indicates resistance to an immunotherapy and a decreased overall survival.

In another aspect, the present invention provides for a method of predicting cancer clinical outcome in a subject in need thereof comprising detecting in a sample obtained from the subject the ratio of TCF7+CD8+ to TCF7-CD8+ T cells, wherein a ratio greater than one indicates sensitivity to an immunotherapy and an increased overall survival and wherein a ratio less than one indicates resistance to an immunotherapy and a decreased overall survival. In certain embodiments, TCF7+CD8+ and TCF7-CD8+ T cells are detected by immunofluorescence.

In certain embodiments, the method further comprises detecting mutations associated with loss of antigen presentation in tumor cells obtained from the subject, wherein detecting a mutation associated with loss of antigen presentation indicates resistance to an immunotherapy and a decreased overall survival. In certain embodiments, the mutations result in the loss of one or more genes or polypeptides selected from the group consisting of B2M, HLA-A, HLA-B, and HLA-C. In certain embodiments, predicting cancer clinical outcome is performed before, after or during treatment with a checkpoint blockade (CPB) therapy.

In another aspect, the present invention provides for a method of enriching for memory/effector CD8+ T cells comprising sorting for CD8+ T cells lacking expression of ENTPD1 and HAVCR2 and/or lacking expression of CD38.

In another aspect, the present invention provides for a method of enriching for exhausted CD8+ T cells comprising sorting for CD8+ T cells that express ENTPD1 and HAVCR2 and/or express CD38.

In certain embodiments, the cells are sorted using antibodies specific to ENTPD1 and HAVCR2 and/or CD38.

In another aspect, the present invention provides for a population of CD8+ T cells, wherein the population of cells comprises CD8+ T cells that lack expression of ENTPD1 and HAVCR2 and/or CD38. The population of cells may be depleted for CD8+ T cells that express ENTPD1 and HAVCR2 and/or CD38. The population of cells may be enriched for CD8+ T cells that lack expression of ENTPD1 and HAVCR2 and/or CD38.

In certain embodiments, the population of CD8+ T cells are modulated to decrease activity or expression of one or more genes or polypeptides selected from the group consisting of: ENTPD1 and HAVCR2; or CCL3, CD38 and HAVCR2; or CD38, CCL3, VCAM1, GOLIM4, HAVCR2, PRDX3, ENTPD1, PTTG1, CCR5, TRAFD1, PDCD1, CXCR6, BATF, PTPN6, LAG3 and CTLA4; or CD38, EPSTI1, GOLIM4, WARS, PDCD1, CCL3, SNAP47, VCAM1, SKA2, HAVCR2, LGALS9, PRDX3, FASLG, ENTPD1, FABP5, SIRPG, LSM2, NDUFB3, TRAFD1, UBE2F, NMI, IFI35, CLTA, MTHFD1, MYO7A, IFI27L2, MCM5, STMN1, ID3, RGS3, SNRPD1, PTTG1 and FIBP; or CD8_B genes listed in Table 6 of US Pat. App. Pub. 2019/0255107.

In certain embodiments, the population of CD8+ T cells are modulated to increase activity or expression one or more genes or polypeptides selected from the group consisting of: TCF7; or TCF7 and IL7R; or TCF7, IL7R, FOSL2, REL, FOXP1, and STAT4; or TCF7, PLAC8, LTB, and CCR7; or TCF7, LEF1, S1PR1, PLAC8, LTB, and CCR7; or TCF7, IL7R, GPR183, and MGAT4A; or TCF7, IL7R, GPR183, LMNA, NR4A3, CD55, AIM1, MGAT4A, PER1, FOSL2, TSPYL2, REL, FAM177A1, YPEL5, TC2N and CSRNP1; or TCF7, IL7R, GPR183, LMNA, NR4A3, CD55, AIM1, MGAT4A, PER1, FOSL2, TSPYL2, REL, FAM177A1, YPEL5, TC2N, CSRNP1, FAM65B, PIK3R1, RGPD6, SKIL, TSC22D2, USP36, FOXP1, EGR1, MYADM, ZFP36L2, FAM102A, RGCC, PDE4B, PFKFB3, FOSB, DCTN6 and BTG2; or CD8_G genes listed in Table 6 of US Pat. App. Pub. 2019/0255107.

In certain embodiments, the one or more genes are modulated with a genetic modifying agent. In certain embodiments, the population of cells comprises activated T cells. In certain embodiments, the population of cells comprises T cells activated with tumor specific antigens. In certain embodiments, the tumor specific antigens are subject specific antigens.

In another aspect, the present invention provides for a pharmaceutical composition comprising the population of cells according to any embodiment herein.

In another aspect, the present invention provides for a method of treating cancer in a subject in need thereof comprising administering an inhibitor of CD39 and an inhibitor of TIM3 or an inhibitor of CD39 and an inhibitor of PD1. The inhibitor of TIM3 may comprise anti-TIM3 antibodies or the inhibitor of PD1 may comprise anti-PD1 antibodies. The inhibitor of CD39 may comprise POM-1.

In another aspect, the present invention provides for a method of treating cancer in a subject in need thereof comprising: predicting cancer clinical outcome based on the ratio of immune cells enriched for expression of responder and non-responder gene signatures; and treating the subject, wherein responders are treated with an immunotherapy comprising checkpoint blockade (CPB) therapy, wherein non-responders are treated with: adoptive cell transfer and optionally checkpoint blockade (CPB) therapy; or an inhibitor of CD39 and an inhibitor of TIM3; or an inhibitor of CD39 and an inhibitor of PD1; or an agent capable of targeting, inhibiting or depleting CD8+ TILs having said non-responder signature and optionally checkpoint blockade (CPB) therapy; or an agent capable of activating, maintaining or increasing CD8+ TILs having said responder signature and optionally checkpoint blockade (CPB) therapy, or wherein non-responders comprising tumors not capable of presenting antigens are treated with a therapy other than checkpoint blockade (CPB) therapy.

In certain embodiments, the adoptive cell transfer comprises: autologous T cells having the responder signature; or autologous T cells specific against tumor antigens, having the responder signature; or autologous T cells transduced with T cell receptors targeting tumor antigens, having the responder signature; or autologous CAR T cells having the responder gene signature; or allogenic T cells having the responder signature; or allogenic T cells specific against tumor antigens, having the responder signature; or allogenic T cells transduced with T cell receptors targeting tumor antigens, having the responder signature; or allogenic CAR T cells having the responder gene signature. In certain embodiments, the autologous T cells are obtained from the subject and cells having the non-responder signature are depleted and/or cells having the responder signature are expanded. In certain embodiments, CAR T cells are enriched for cells having a responder signature or depleted for cells having a non-responder signature. In certain embodiments, the agent capable of targeting, inhibiting or depleting CD8+ TILs having a non-responder signature comprises: an agent capable of binding to a cell surface or secreted CD8+ T cell non-responder signature gene; or an agent capable of reducing the expression or activity of the non-responder signature. In certain embodiments, the agent capable of activating, maintaining or increasing CD8+ TILs having a responder signature comprises an agent capable of increasing or activating the expression of the responder signature. In certain embodiments, checkpoint blockade (CPB) therapy comprises anti-CTLA4, anti-PD-L1, anti-PD1 therapy or combinations thereof.

In another aspect, the present invention provides for a method of treating cancer in a subject in need thereof comprising administering an agent capable of increasing the expression or activity of one or more genes or polypeptides selected from the group consisting of TCF7, IL7R, GPR183, LMNA, NR4A3, CD55, AIM1, MGAT4A, PER1, FOSL2, TSPYL2, REL, FAM177A1, YPEL5, TC2N, CSRNP1, FAM65B, PIK3R1, RGPD6, SKIL, TSC22D2, USP36, FOXP1, STAT4, PLAC8, LTB LEF1, S1PR1, EGR1, MYADM, ZFP36L2, FAM102A, RGCC, PDE4B, PFKFB3, FOSB, DCTN6 and BTG2 in combination with checkpoint blockade therapy.

In another aspect, the present invention provides for a method of treating cancer in a subject in need thereof comprising administering an agent capable of reducing the expression or activity of one or more genes or polypeptides selected from the group consisting of CD38, CCL3, VCAM1, GOLIM4, HAVCR2, PRDX3, ENTPD1, PTTG1, CCR5, TRAFD1, PDCD1, CXCR6, BATF, PTPN6, LAG3 and CTLA4 in combination with checkpoint blockade therapy.

In another aspect, the present invention provides for a method of treating cancer in a subject in need thereof comprising administering CD8+ T cells expressing a gene signature comprising of one or more genes selected from the group consisting of TCF7, IL7R, GPR183, LMNA, NR4A3, CD55, AIM1, MGAT4A, PER1, FOSL2, TSPYL2, REL, FAM177A1, YPEL5, TC2N, CSRNP1, FAM65B, PIK3R1, RGPD6, SKIL, TSC22D2, USP36, FOXP1, STAT4, PLAC8, LTB LEF1, S1PR1, EGR1, MYADM, ZFP36L2, FAM102A, RGCC, PDE4B, PFKFB3, FOSB, DCTN6 and BTG2 in combination with checkpoint blockade therapy.

In certain embodiments, agent comprises a therapeutic antibody, antibody fragment, antibody-like protein scaffold, aptamer, protein, genetic modifying agent or small molecule.

In another aspect, the present invention provides for a method of monitoring a subject in need thereof undergoing treatment with checkpoint blockade (CPB) therapy, said method comprising detecting in a tumor sample obtained from the subject the expression or activity of a gene signature comprising one or more genes or polypeptides selected from the group consisting of: ENTPD1 and HAVCR2; or CCL3, CD38 and HAVCR2; or CD38, CCL3, VCAM1, GOLIM4, HAVCR2, PRDX3, ENTPD1, PTTG1, CCR5, TRAFD1, PDCD1, CXCR6, BATF, PTPN6, LAG3 and CTLA4; or CD38, EPSTI1, GOLIM4, WARS, PDCD1, CCL3, SNAP47, VCAM1, SKA2, HAVCR2, LGALS9, PRDX3, FASLG, ENTPD1, FABP5, SIRPG, LSM2, NDUFB3, TRAFD1, UBE2F, NMI, IFI35, CLTA, MTHFD1, MYO7A, IFI27L2, MCM5, STMN1, ID3, RGS3, SNRPD1, PTTG1 and FIBP; or CD8_B genes listed in Table 6 of US Pat. App. Pub. 2019/0255107, wherein the treatment is adjusted if the signature is increased in CD8+ TILs after treatment.

In another aspect, the present invention provides for a method of monitoring a subject in need thereof undergoing treatment with checkpoint blockade (CPB) therapy, said method comprising detecting in a tumor sample obtained from the subject the expression or activity of a gene signature comprising one or more genes or polypeptides selected from the group consisting of: TCF7; or TCF7 and IL7R; or TCF7, IL7R, FOSL2, REL, FOXP1, and STAT4; or TCF7, PLAC8, LTB, and CCR7; or TCF7, LEF1, S1PR1, PLAC8, LTB, and CCR7; or TCF7, IL7R, GPR183, and MGAT4A; or TCF7, IL7R, GPR183, LMNA, NR4A3, CD55, AIM1, MGAT4A, PER1, FOSL2, TSPYL2, REL, FAM177A1, YPEL5, TC2N and CSRNP1; or TCF7, IL7R, GPR183, LMNA, NR4A3, CD55, AIM1, MGAT4A, PER1, FOSL2, TSPYL2, REL, FAM177A1, YPEL5, TC2N, CSRNP1, FAM65B, PIK3R1, RGPD6, SKIL, TSC22D2, USP36, FOXP1, EGR1, MYADM, ZFP36L2, FAM102A, RGCC, PDE4B, PFKFB3, FOSB, DCTN6 and BTG2; or CD8_G genes listed in Table 6 of US Pat. App. Pub. 2019/0255107, wherein the treatment is adjusted if the signature is decreased in CD8+ TILs after treatment.

In another aspect, the present invention provides for a method of manufacturing cells for use in adoptive cell transfer comprising: obtaining CD8+ T cells; and depleting cells having a CPB therapy non-responder signature as described herein or selecting for cells having a CPB therapy responder signature as described herein. The method may further comprise expanding cells having a responder signature. The method may further comprise activating the cells. The method may further comprise expressing a chimeric antigen receptor (CAR) or an endogenous T cell receptor (TCR) in the cells.

In another aspect, the present invention provides for a kit comprising reagents to detect at least one gene or polypeptide according to a gene signature as described herein. The kit may comprise at least one antibody, antibody fragment, or aptamer. The kit may comprise primers and/or probes or fluorescently bar-coded oligonucleotide probes for hybridization to RNA.

### Identifying T Cell Subtypes Responsive to CPB Therapy

RNA profiles from populations and single-cell CD8⁺ tumor-infiltrating lymphocytes (TILs) can change after Tim-3/PD-1 blockade. There were greater transcriptional changes in Tim-3-PD-1⁻ compared to Tim-3⁺PD-1⁺CD8⁺ TILs, identifying three novel subsets of Tim-3⁻PD-1⁻CD8⁺ TILs that have features of naïve, effector, or memory-precursor T cells. Following Tim-3/PD-1 blockade, the proportion of memory-precursor-like and effector-like TIL subsets increases relative to the naïve-like subset. Tcf7 can be a regulator of the memory-precursor-like subset and show that different immunotherapies fail in its absence. The memory-precursor-like subset shares features with CD8⁺ T cells that are predictive of better prognosis and of response to checkpoint blockade in patients. The findings provide critical insight into development of the effector CD8⁺ T cell response after immunotherapy.

It is an objective of the present invention to identify CD8⁺ TIL subtypes responsive to checkpoint blockade therapy. It is another objective of the present invention to detect gene signatures and biomarkers specific to the CD8⁺ TIL subtypes, whereby cells may be detected and isolated. It is another objective of the present invention to provide for adoptive cell transfer methods for treatment of a cancer by transferring more functional CD8⁺ TILs. It is another objective of the present invention to provide for treatment of a cancer by modulating CD8⁺ T cells to be more functional. It is another objective of the present invention to improve immunotherapy treatment.

In one aspect, the present invention provides for an isolated CD8+ T cell characterized in that the CD8+ T cell comprises: expression of SLAMF7 and does not express CD62L, CX3CR1, TIM3 and PD1. The isolated CD8+ T cell may be further characterized in that the CD8+ T cell does not express KLRG1.

In another aspect, the present invention provides for an isolated CD8+ T cell characterized in that the CD8+ T cell comprises: expression of SLAMF7 and CX3CR1 and does not express CD62L, TIM3 and PD1. The isolated CD8+ T cell may be further characterized in that the CD8+ T cell expresses KLRG1. The isolated CD8+ T cell may be further characterized in that the CD8+ T cell does not express KLRG1. Not being bound by a theory the CD62L− Slamf7+CX3CR1+CD8+ T cell may be further characterized as a KLRG1+ or KLRG1− cell.

The isolated CD62L− Slamf7+CX3CR1− CD8+ T cell and isolated CD62L-Slamf7+CX3CR1+ CD8+ T cell may be further characterized by a gene signature comprising one or more genes or polypeptides in Table 5 of US Pat. App. Pub. 2019/0255107. The isolated CD62L− Slamf7+ CX3CR1− CD8+ T cell may be further characterized in that the CD8+ T cell also expresses or does not express one or more genes or polypeptides selected from Table 5 of US Pat. App. Pub. 2019/0255107. The isolated CD62L− Slamf7+CX3CR1+CD8+ T cell may be further characterized in that the CD8+ T cell also expresses or does not express one or more genes or polypeptides selected from Table 5 of US Pat. App. Pub. 2019/0255107. Table 5 of US Pat. App. Pub. 2019/0255107 list genes differentially expressed between the two CD62L− Slamf7+ subtypes described herein. Thus, the signature of genes up and down regulated in Table 5 of US Pat. App. Pub. 2019/0255107 may be used to further distinguish between each subtype. In certain embodiments, the overall signatures or subset of signature genes listed in Table 65 of US Pat. App. Pub. 2019/0255107 may be used to identify each subtype.

The gene signature in Table 5 of US Pat. App. Pub. 2019/0255107 comprises one or more transcription factors that may be key regulators or drivers of the phenotype of the two CD62L− Slamf7+ subtypes. Transcription factors may indicate key pathways for modulating activity of the cells and may be therapeutic targets. The CD62L− Slamf7+CX3CR1− CD8+ T cell may comprise higher expression of one or more transcription factors selected from the group consisting of Tcf7, Egr2, Zfp827, Satb1, Zfp512, Irf8, Re1b, Sp140, Myb, Id3, Hes6, Fos, Ikzf2 and Myc relative to the CD62L− Slamf7+CX3CR1+CD8+ T cell. The CD62L-Slamf7+CX3CR1+CD8+ T cell may comprise higher expression of one or more transcription factors selected from the group consisting of Bhlhe40, Klf2, Zeb2, Prdm1, Arntl, Ets1, Junb, Id2, Hivep2, Rora, Nr1d2, Meis2, Arnt, Nr4a1, Meis3, Zmiz1, Vezf1, Nfe2l1, Mxi1, Rxra and Creb5 relative to the CD62L− Slamf7+CX3CR1− CD8+ T cell.

In another aspect, the present invention provides for an isolated CD8+ T cell characterized in that the CD8+ T cell comprises: expression of CD62L and does not express SLAMF7, CX3CR1, KLRG1, TIM3 and PD1.

The isolated CD62L− Slamf7+ CX3CR1− CD8+ T cell, isolated CD62L− Slamf7+CX3CR1+ CD8+ T cell, and isolated CD62Lhi Slamf7−CD8+ T cell may be further characterized by a gene signature comprising one or more genes or polypeptides in Table 3 of US Pat. App. Pub. 2019/0100801. Table 3 of US Pat. App. Pub. 2019/0100801 lists genes differentially expressed in one or more of the CD8+ T cell subtypes described herein relative to one or more of another subtype (i.e. genes differentially expressed relative to all three subtypes). Thus, genes up and down regulated in one subtype relative to the other subtypes listed in Table 3 of US Pat. App. Pub. 2019/0100801 may be used to further distinguish between each subtype. In certain embodiments, the overall signatures or subset of signature genes listed in Table 3 of US Pat. App. Pub. 2019/0100801 may be used to identify each subtype.

The isolated CD8+ T cell according to any embodiment herein, may be a human cell. The isolated CD8+ T cell may be a CAR T cell. The CAR T cell may be autologous or allogenic. In preferred embodiments, the isolated CD8+ T cell may be autologous for a subject suffering from cancer. The isolated CD8+ T cell may express an exogenous CAR or TCR. The isolated CD8+ T cell may display tumor specificity.

In another aspect, the present invention provides for a method for detecting or quantifying CD8+ T cells in a biological sample of a subject, or for isolating CD8+ T cells from a biological sample of a subject, the method comprising detecting or quantifying in a biological sample of the subject CD8+ T cells as defined in any embodiment herein, or isolating from the biological sample CD8+ T cells as defined in any embodiment herein. The CD8+ T cells may be detected, quantified or isolated using a set of markers comprising: SLAMF7, CD62L, CX3CR1, and PD1; or SLAMF7, CD62L, CX3CR1, and TIM3; or SLAMF7, CD62L, CX3CR1, KLRG1 and PD1; or SLAMF7, CD62L, CX3CR1, KLRG1 and TIM3; or any of the above markers and one or more genes or polypeptides selected from the group consisting of Table 3 of US Pat. App. Pub. 2019/0100801 or any of the above markers and one or more genes or polypeptides selected from the group consisting of Table 5 of US Pat. App. Pub. 2019/0255107.

The CD8+ T cells may be detected, quantified or isolated using a technique selected from the group consisting of flow cytometry, mass cytometry, fluorescence activated cell sorting, fluorescence microscopy, affinity separation, magnetic cell separation, microfluidic separation, and combinations thereof. The technique may employ one or more agents capable of specifically binding to one or more gene products expressed or not expressed by the CD8+ T cells, preferably on the cell surface of the CD8+ T cells. The one or more agents may be one or more antibodies.

The biological sample may be a tumor sample obtained from a subject in need thereof and the CD8+ T cells may be CD8+ tumor infiltrating lymphocytes (TIL). The biological sample may comprise ex vivo or in vitro CD8+ T cells. The biological sample may be treated with an antigen. The biological sample may be treated with a differentiation agent. The differentiating agent may be a cytokine. The cytokine may be an agent known to effect T cell differentiation. The biological sample may be treated with an agent capable of increasing the proportion of Slamf7+CX3CR1−CD62L− cells as defined herein. The agent may be any agent predicted to affect the function or gene expression of any of the cells described herein. The agent may affect the ratio of cells in a population of cells. The agent may be a drug candidate. The agent may be a drug predicted to induce a gene signature described herein. The agent may be a drug predicted to reduce a gene signature described herein. Agents may be those predicted in silico (e.g., CMAP) or screened from a known compound library to affect a gene signature. The agent may also include drugs targeting a specific subtype for reducing said subtype. Not being bound by a theory, targeting a subtype for removal can increase the proportion of another subtype. Drugs targeting a specific subtype may include antibody drug conjugates specific for a subtype specific surface marker. The agent may also maintain a specific subtype, thus increasing the proportion of that subtype in a biological sample. The agent may be selected to activate or express a transcription factor. In other embodiments, the agent may be selected to repress a transcription factor. In certain example embodiments, the agent may include an agent selected to activate TCF7. In certain example embodiments, the agent may include an agent selected to downregulate expression of Bhlhe40, also known as DEC1, to maintain a basal level.

In another aspect, the present invention provides for a population of CD8+ T cells comprising CD8+ T cells as defined in any embodiment herein or isolated according to a method of any embodiment herein. The population may comprise greater than 5%, 10%, 20%, 30%, 40%, 50%, 60%, 70%, 80%, 90% or 95% of CD8+ T cells as defined in any embodiment herein. In certain embodiments, the population of cells is less than 5% of any one cell type, such as when cells are directly isolated from a patient. Not being bound by a theory, a population of cells isolated from a patient will include a heterogeneous population of cells, such that specific cell subtypes make up less than a majority of the total cells (e.g., less than 30%, 20%, 10%, 5%). In certain embodiments, a subtype of cells is expanded or enriched ex vivo to obtain a non-naturally occurring cell population enriched for certain cell types. The population of cells may comprise CD8⁺ T cells as defined in any embodiment herein. In preferred embodiments, the population of cells are characterized in that the population comprises CD8⁺ T cells that express SLAMF7 and that do not express CD62L, CX3CR1, TIM3 and PD1 (CD62L⁻ Slamf7⁺ CX3CR1⁻). In other preferred embodiments, the population of cells are characterized in that the population comprises CD8⁺ T cells that express CD62L and that do not express SLAMF7, CX3CR1, KLRG1, TIM3 and PD1 (CD62L^{hi} Slamf7⁻). Not being bound by a theory, the CD62L^{hi} Slamf7⁻ CD8⁺ T cells may be the progenitor population that gives rise to the CD62L⁻ Slamf7⁺ CX3CR1⁻ CD8⁺ T cells. Not being bound by a theory, a population of progenitor cells may provide for a population of cells capable of differentiating into polyfunctional cells capable of controlling or eliminating cancer in vivo (e.g., for use in adoptive cell transfer).

The population of cells may be enriched for the CD8⁺ T cells that express SLAMF7 and that do not express CD62L, CX3CR1, TIM3 and PD1 or for the CD8⁺ T cells that express CD62L and that do not express SLAMF7, CX3CR1, KLRG1, TIM3 and PD1. The enriched population of cells may comprise CAR T cells. The population of enriched cells may comprise CD8⁺ T cells autologous for a subject suffering from cancer. The population of cells may express an exogenous CAR or TCR. Not being bound by a theory, the enriched cell types may be more effective in targeting a tumor expressing antigens specific for the CAR or TCR than a population of unenriched T cells. Not being bound by a theory, unenriched T cells may include suppressive cell types.

The population of cells may display tumor specificity. The population of cells may comprise expanded cells. The population of cells may comprise activated CD8⁺ T cells. The population of cells may comprise T cells activated with tumor specific antigens. The tumor specific antigens are subject specific antigens.

The population of CD8⁺ T cells may comprise cells modified to knockout or downregulate expression of one or more genes selected from the group consisting of Bhlhe40 (DEC1), Klf2, Zeb2, Prdm1, Arntl, Ets1, Junb, Id2, Hivep2, Rora, Nr1d2, Meis2, Arnt, Nr4a1, Meis3, Zmiz1, Vezf1, Nfe2l1, Mxi1, Rxra and Creb5. The population of cells may comprise cells modified to downregulate expression of Bhlhe40, such that the population of cells maintain at least a basal level of Bhlhe40 expression. As used herein, the term “basal” refers to the minimum expression level of a gene in a cell (e.g., T cell). Not being bound by a theory, at least basal expression of Bhlhe40 is required for proper function of the CD62L− Slamf7+CX3CR1⁻ cells. The population of CD8⁺ T cells may comprise cells modified to increase expression of one or more genes selected from the group consisting of Tcf7, Egr2, Zfp827, Satb1, Zfp512, Irf8, Re1b, Sp140, Myb, Id3, Hes6, Fos, Ikzf2 and Myc. The population of cells may comprise cells modified to increase expression of Tcf7. The cells may be modified by any method known in the art. In preferred embodiments, the cells are modified with a CRISPR system. Not being bound by a theory, modifying the ability of the CD8⁺ T cells to express one or more genes selected from the group consisting of Bhlhe40 (DEC1), Klf2, Zeb2, Prdm1, Arntl, Ets1, Junb, Id2, Hivep2, Rora, Nr1d2, Meis2, Arnt, Nr4a1, Meis3, Zmiz1, Vezf1, Nfe2l1, Mxi1, Rxra and Creb5 may prevent the cells from differentiating to nonfunctional cells and/or suppressive cells or from differentiating to CD8⁺ T cells characterized by expression of SLAMF7 and CX3CR1 and lack of expression of CD62L, TIM3 and PD1 (CD62L⁻ Slamf7⁺ CX3CR1⁺).

In another aspect, the present invention provides for a pharmaceutical composition comprising the CD8⁺ T cell as defined in any embodiment herein or the CD8⁺ T cell population as defined in any embodiment herein.

In another aspect, the present invention provides for a method for treating or preventing cancer comprising administering to a subject in need thereof the pharmaceutical composition as described herein. The method may comprise: isolating from a biological sample of the subject a CD8⁺ T cell or CD8⁺ T cell population; in vitro expanding the CD8⁺ T cell or CD8⁺ T cell population; and administering the in vitro expanded CD8⁺ T cell or CD8⁺ T cell population to the subject. The method may further comprise enriching the expanded cells for CD8⁺ T cells that express SLAMF7 and that do not express CD62L, CX3CR1, TIM3 and PD1 (CD62L⁻ Slamf7⁺ CX3CR1⁻). The method may further comprise enriching the expanded cells for CD8⁺ T cells that express CD62L and that do not express SLAMF7, CX3CR1, KLRG1, TIM3 and PD1 (CD62L^{hi} Slamf7⁻). The pharmaceutical composition may be administered after ablation therapy or before surgery. Not being bound by a theory, providing the pharmaceutical composition before surgery may shrink the tumor before it is removed. Not being bound by a theory, providing the pharmaceutical composition after ablation therapy or lymphodepletion may eliminate suppressor cells that can attenuated the activity of the transferred cells.

The method of treatment according to any embodiment, may further comprise administering a checkpoint blockade therapy. The checkpoint blockade therapy may comprise anti-TIM3, anti-CTLA4, anti-PD-L1, anti-PD1, anti-TIGIT, anti-LAG3, or combinations thereof. Not being bound by a theory, a treatment that increases the number or activity of cells that express SLAMF7 and that do not express CD62L, CX3CR1, TIM3 and PD1 (CD62L⁻ Slamf7⁺ CX3CR1⁻) may have an improved response to checkpoint blockade therapy.

In another aspect, the present invention provides for a method for identifying an immunomodulant capable of modulating one or more phenotypic aspects of the CD8⁺ T cell as defined in any embodiment herein or the CD8⁺ T cell population as defined in any embodiment herein, comprising: applying a candidate immunomodulant to the CD8⁺ T cell or CD8⁺ T cell population; and detecting modulation of one or more phenotypic aspects of the CD8⁺ T cell or CD8⁺ T cell population by the candidate immunomodulant, thereby identifying the immunomodulant.

In another aspect, the present invention provides for an immunomodulant capable of modulating one or more phenotypic aspects of the CD8⁺ T cell as defined in any embodiment herein or the CD8⁺ T cell population as defined in any embodiment herein, such as an immunomodulant identified using the method as defined above. The immunomodulant may be capable of modulating the proliferation, differentiation, maturation, migration, cytokine expression, cytotoxicity and/or viability of the CD8⁺ T cell or CD8⁺ T cell population. The immunomodulant may be capable of inducing or repressing the proliferation, differentiation, maturation, migration, cytokine expression, cytotoxicity and/or viability of the CD8⁺ T cell or CD8⁺ T cell population. The immunomodulant may comprise a therapeutic antibody, antibody fragment, antibody-like protein scaffold, aptamer, protein, CRISPR system or small molecule.

In another aspect, the present invention provides for a pharmaceutical composition comprising the immunomodulant as defined in any embodiment herein.

In another aspect, the present invention provides for a method for determining the CD8⁺ T cell status of a subject, or for diagnosing, prognosing or monitoring a disease comprising an immune component in a subject, the method comprising detecting or quantifying in a biological sample of the subject CD8⁺ T cells as defined in any embodiment herein. In certain embodiments, detecting or quantifying the CD8⁺ T cells in a biological sample of the subject may comprise detecting Tcf7. The disease may be cancer, an autoimmune disease or a chronic infection (e.g., viral infection). The CD8⁺ T cell status of the subject may be determined before and after therapy, whereby the efficacy of the therapy is determined or monitored. The therapy may be, but is not limited to, an immunotherapy, innate immune agonists, vaccines, chemotherapies, and small molecules. Not being bound by a theory, determining the CD8⁺ T cell status by detection of the subtypes described herein after a treatment may indicate that the patient requires an increase in a specific subtype (e.g., adoptive cell transfer). The immunotherapy may comprise checkpoint blockade therapy. Not being bound by a theory, determining the CD8+ T cell status of a subject may indicate that the subject will respond to a checkpoint blockade therapy. In certain embodiments, detecting CD62L− Slamf7+CX3CR1− CD8+ T cells indicates an improved prognosis. In certain embodiments, the proportion of CD8+ subtypes is determined and subjects having a higher proportion of CD62L− Slamf7+CX3CR1− CD8+ T cells as compared to other subjects have an improved prognosis. In certain embodiments, detecting CD62L− Slamf7+CX3CR1− CD8+ T cells indicates that a subject can respond to an immunotherapy. In certain embodiments, the proportion of CD8+ subtypes is determined and subjects having a higher proportion of CD62L− Slamf7+ CX3CR1− CD8+ T cells as compared to other subjects will respond better to an immunotherapy. In certain embodiments, detecting CD62L− Slamf7+ CX3CR1− CD8+ T cells may comprise detecting cells positive for Tcf7.

In another aspect, the present invention provides for a method of identifying T cell receptors (TCR) specific for an antigen comprising isolating CD8⁺ T cells that express SLAMF7 and that do not express CD62L, CX3CR1, TIM3 and PD1 (CD62L− Slamf7⁺ CX3CR1⁻) and identifying TCRs expressed by the isolated cells. The cells may be isolated from a tumor. The antigen may be a tumor specific antigen. Not being bound by a theory, the CD62L⁻ Slamf7⁺ CX3CR1⁻ CD8⁺ cells isolated from a tumor express tumor specific TCRs. Not being bound by a theory, the antigen determining regions of these TCRs may be used to generate tumor specific CARs.

In another aspect, the present invention provides for a method of preparing a CAR T cell specific for a tumor antigen comprising identifying TCRs according to any embodiment herein and generating a CAR T cell comprising the antigen-binding portion of the TCR identified.

In another aspect, the present invention provides for a method of preparing cells for use in adoptive cell transfer comprising: obtaining CD8⁺ T cells; and enriching for CD8⁺ T cells that express SLAMF7 and that do not express CD62L, CX3CR1, TIM3 and PD1 (CD62L⁻ Slamf7⁺ CX3CR1⁻) or for CD8⁺ T cells that express CD62L and that do not express SLAMF7, CX3CR1, KLRG1, TIM3 and PD1 (CD62L^{hi} Slamf7⁻). The method may further comprise expanding the cells. The method may further comprise activating the cells. The CD8⁺ T cells may further comprise a CAR. The CD8⁺ T cells may be autologous TILs. The method may further comprise treating the CD8⁺ T cells with an agonist of a transcription factor selected from the group consisting of Tcf7, Egr2, Zfp827, Satb1, Zfp512, Irf8, Re1b, Sp140, Myb, Id3, Hes6, Fos, Ikzf2 and Myc. In preferred embodiments, the transcription factor is Tcf7. The Tcf7 agonist may comprise an agonist of Wnt/beta-catenin signaling.

In another aspect, the present invention provides for a method of preparing cells for use in adoptive cell transfer comprising: obtaining CD8⁺ T cells; and treating the CD8⁺ T cells with an agonist of a transcription factor selected from the group consisting of Tcf7, Egr2, Zfp827, Satb1, Zfp512, Irf8, Re1b, Sp140, Myb, Id3, Hes6, Fos, Ikzf2 and Myc. In preferred embodiments, the transcription factor is Tcf7. The Tcf7 agonist may comprise an agonist of Wnt/beta-catenin signaling. The method may further comprise expanding the cells. The method may further comprise activating the cells. The CD8⁺ T cells may further comprise a CAR. The CD8⁺ T cells may be autologous TILs.

In another aspect, the present invention provides for a method of detecting a CD8⁺ T cell checkpoint blockade (CPB) therapy gene signature in a tumor comprising detecting in CD8⁺ T cells obtained from a subject in need thereof the expression or activity of a signature comprising one or more genes selected from Table 1 of US Pat. App. Pub. 2019/0255107 or Table 1 herein.

In another aspect, the present invention provides for a method for determining the CD8⁺ T cell status of a subject suffering from cancer, said method comprising detecting in Tim-3+PD-1 CD8⁺ TILs from the subject a Tim-3⁺PD-1⁺ CPB gene signature and/or detecting in Tim-3-PD-1⁻ CD8⁺ TILs from the subject a Tim-3⁻PD-1⁻ CPB gene signature, said gene signatures comprising one or more genes selected from Table 1 of US Pat. App. Pub. 2019/0255107. In certain embodiments, the subject is undergoing or has received CPB treatment and an increase in the Tim-3⁺PD-1⁺ and/or Tim-3⁻PD-1⁻ CPB gene signature as compared to a reference level before treatment indicates an enhanced CD8⁺ T cell immune response.

In another aspect, the present invention provides for a method for determining the CD8⁺ T cell status of a subject suffering from cancer, said method comprising detecting in CD8⁺ TILs from the subject a gene signature comprising one or more genes selected from Table 1 herein. In certain embodiments, the subject is undergoing or has received CPB treatment and upregulation of the one or more genes as compared to a reference level before treatment indicates an enhanced CD8⁺ T cell immune response.

In certain embodiments, the CPB treatment comprises anti-PD1, anti-TIM3, anti-CTLA4, anti-PD-L1, anti-TIGIT, anti-LAG3, or combinations thereof.

In another aspect, the present invention provides for a method of preparing cells for use in adoptive cell transfer comprising: increasing expression or activity of one or more genes selected from Table 1 herein in CD8⁺ T cells; or modulating expression or activity of one or more genes selected from Table 1 of US Pat. App. Pub. 2019/0255107 in CD8⁺ T cells, wherein the genes are modulated in Tim-3⁺PD-1⁺ CD8⁺ and/or Tim-3⁻PD-1⁻ CD8⁺ T cells according to Table 1 of US Pat. App. Pub. 2019/0255107. In certain embodiments, the method further comprises expanding the cells. In certain embodiments, the method further comprises activating the cells. In certain embodiments, the method further comprises the CD8⁺ T cells are CAR T cells. In certain embodiments, the method further comprises the CD8⁺ T cells are autologous TILs.

In certain embodiments, the expression or activity of the one or more genes is modulated by treating the CD8⁺ T cells with an agent, said agent comprising a small molecule, genetic modifying agent, therapeutic antibody, antibody fragment, antibody-like protein scaffold, aptamer or protein. The genetic modifying agent may comprise a CRISPR system, a zinc finger nuclease system, a TALEN, or a meganuclease.

In another aspect, the present invention provides for a method of treating cancer in a subject in need thereof comprising administering to the subject cells prepared according to any embodiment herein.

In another aspect, the present invention provides for a method of identifying an immunomodulant capable of enhancing a CD8⁺ T cell immune response, comprising: applying a candidate immunomodulant to a population of CD8⁺ T cells; and (a) detecting increased expression or activity of one or more genes selected from Table 2 herein in the CD8⁺ T cells; and/or (b) detecting differential expression or activity of one or more genes selected from Table 1 of US Pat. App. Pub. 2019/0255107 in the CD8⁺ T cells, wherein the genes are differentially expressed in Tim-3⁺PD-1⁺ CD8⁺ and/or Tim-3⁻PD-1⁻ CD8⁺ T cells according to Table 1 of US Pat. App. Pub. 2019/0255107, thereby identifying an immunomodulant.

In another aspect, the present invention provides for a kit comprising reagents to detect at least one gene or polypeptide as defined in any embodiment herein.

An aspect of the invention provides the immune cell or immune cell population as taught herein for use in immunotherapy, such as adoptive immunotherapy, such as adoptive cell transfer. Also provided is a method of treating a subject in need thereof, particularly in need of immunotherapy, such as adoptive immunotherapy, such as adoptive cell transfer, comprising administering to said subject the immune cell or immune cell population as taught herein. Further provided is use of the immune cell or immune cell population as taught herein for the manufacture of a medicament for immunotherapy, such as adoptive immunotherapy, such as adoptive cell transfer. In certain embodiments, the immune cell is a T-cell, such as a CD8⁺ T-cell. In certain embodiments, the immunotherapy, adoptive immunotherapy or adoptive cell transfer may be for treating a proliferative disease, such as tumor or cancer, or a chronic infection, such as chronic viral infection.

In certain embodiments, an immune cell suitable for immunotherapy, such as a CD8⁺ T-cell, displays tumor specificity, more particularly displays specificity to a tumor antigen. In certain embodiments, an immune cell suitable for immunotherapy, such as a CD8⁺ T-cell, displays specificity to an antigen of an infectious agent, for example displays viral antigen specificity. In certain embodiments, an immune cell suitable for immunotherapy, such as a CD8⁺ T-cell, has been isolated from a tumor of a subject, preferably the cell is a tumor infiltrating lymphocyte (TIL). In certain embodiments, an immune cell suitable for immunotherapy, such as a CD8⁺ T-cell, comprises a chimeric antigen receptor (CAR). Such cell can also be suitably denoted as having been engineered to comprise or to express the CAR. In certain embodiments, the CAR comprises an extracellular antigen-binding element (or portion or domain) configured to specifically bind to a target antigen, a transmembrane domain, and an intracellular signaling domain. In certain embodiments, the intracellular signaling domain comprises a primary signaling domain and/or a costimulatory signaling domain. In certain embodiments, the CAR comprises the antigen-binding element, costimulatory signaling domain and primary signaling domain (such as CD3 zeta portion) in that order. In certain embodiments, the antigen-binding element comprises, consists of or is derived from an antibody, for example, the antigen-binding element is an antibody fragment. In certain embodiments, the antigen-binding element is derived from, for example is a fragment of, a monoclonal antibody, such as a human monoclonal antibody or a humanized monoclonal antibody. In certain embodiments, the antigen-binding element is a single-chain variable fragment (scFv). In certain preferred embodiments, the target antigen is selected from a group consisting of: CD19, BCMA, CLL-1, MAGE A3, MAGE A6, HPV E6, HPV E7, WT1, CD22, CD171, ROR1, MUC16, and SSX2. In certain preferred embodiments, the target antigen is CD19. In certain embodiments, the transmembrane domain is derived from the most membrane proximal component of the endodomain. In certain embodiments, the transmembrane domain is not CD3 zeta transmembrane domain. In certain embodiments, the transmembrane domain is a CD8α transmembrane domain or a CD28 transmembrane domain, preferably CD28 transmembrane domain. In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain preferred embodiments, the primary signaling domain comprises a functional signaling domain of CD3ζ. In certain embodiments, the one or more costimulatory signaling domains comprise a functional signaling domain of a protein selected, each independently, from the group consisting of: CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD 11a, LFA-1, ITGAM, CD11b, ITGAX, CD 11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain preferred embodiments, the one or more costimulatory signaling domains comprise a functional signaling domain of a protein selected, each independently, from the group consisting of: 4-1BB, CD27, and CD28. In certain preferred embodiments, the costimulatory signaling domain comprises a functional signaling domain of CD28. In certain embodiments, the CAR comprises an anti-CD19 scFv, an intracellular domain of a CD3ζ chain, and a signaling domain of CD28. In certain preferred embodiments, the CD28 sequence is as set forth in Genbank identifier NM_006139 (sequence version 1, 2 or 3) starting with the amino acid sequence IEVMYPPPY and continuing all the way to the carboxy-terminus of the protein. In certain preferred embodiments, the CAR is as included in KTE-C19 (axicabtagene ciloleucel) anti-CD19 CAR-T therapy product in development by Kite Pharma, Inc. In certain embodiments, an immune cell suitable for immunotherapy, such as a CD8⁺ T-cell, comprises an exogenous T-cell receptor (TCR). Such cell can also be suitably denoted as having been engineered to comprise or to express the TCR.

In certain embodiments, an immune cell suitable for immunotherapy, such as a CD8⁺ T-cell, may be further genetically modified, such as gene edited, i.e., a target locus of interest in the cell may be modified by a suitable gene editing tool or technique, such as without limitation CRISPR, TALEN or ZFN. An aspect relates to an immune cell obtainable by or obtained by said gene editing method, or progeny thereof, wherein the cell comprises a modification of the target locus not present in a cell not subjected to the method. Another aspect relates to a cell product from said cell or progeny thereof, wherein the product is modified in nature or quantity with respect to a cell product from a cell not subjected to the gene editing method. A further aspect provides an immune cell comprising a gene editing system, such as a CRISPR-Cas system, configured to carry out the modification of the target locus.

In certain preferred embodiments, the cell may be edited using any CRISPR system and method of use thereof as described herein. In certain preferred embodiments, cells are edited ex vivo and transferred to a subject in need thereof.

Further genetically modifying, such as gene editing, of the cell may be performed for example (1) to insert or knock-in an exogenous gene, such as an exogenous gene encoding a CAR or a TCR, at a preselected locus in the cell; (2) to knock-out or knock-down expression of an endogenous TCR in the cell; (3) to disrupt the target of a chemotherapeutic agent in the cell; (4) to knock-out or knock-down expression of an immune checkpoint protein or receptor in the cell; (5) to knock-out or knock-down expression of other gene or genes in the cell, the reduced expression or lack of expression of which can enhance the efficacy of adoptive therapies using the cell; (6) to knock-out or knock-down expression of an endogenous gene in a cell, said endogenous gene encoding an antigen targeted by an exogenous CAR or TCR; (7) to knock-out or knock-down expression of one or more MHC constituent proteins in the cell; (8) to activate a T cell, and/or increase the differentiation and/or proliferation of functionally exhausted or dysfunctional CD8⁺ T cells; and/or (9) to modulate CD8⁺ T cells, such that CD8⁺ T cells have increased resistance to exhaustion or dysfunction. In certain preferred embodiments, the cell may be edited to produce any one of the following combinations of the modifications set forth above: (1) and (2); (1) and (4); (2) and (4); (1), (2) and (4); (1) and (7); (2) and (7); (4) and (7); (1), (2) and (7); (1), (4) and (7); (1), (2), (4) and (7); optionally adding modification (8) or (9) to any one of the preceding combinations. In certain preferred embodiments, the targeted immune checkpoint protein or receptor is PD-1, PD-L1 and/or CTLA-4. In certain preferred embodiments, the targeted endogenous TCR gene or sequence may be TRBC1, TRBC2 and/or TRAC. In certain preferred embodiments, the targeted MHC constituent protein may be HLA-A, B and/or C, and/or B2M. In certain embodiments, the cell may thus be multiply edited (multiplex genome editing) to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

### CD5L Antagonists and Uses Thereof

In some embodiments, the T cell described herein can express or be capable of expressing a CD5L molecule. CD5L antagonists can reduce or eliminate CD5L expression, singaling, binding its ligand, or any other functionality. In one aspect, the present invention provides for an antagonist against the function or signaling of one or more of a CD5L monomer, a CD5L:CD5L homodimer, and a CD5L:p40 heterodimer. In certain embodiments, the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer. In certain embodiments, the antagonist is an antibody, or an antigen binding fragment or equivalent thereof, that interacts with (e.g., specifically binds with) Il12rb1. In certain embodiments, the antibody is a polyclonal antibody, a monoclonal antibody, a chimeric antibody, a human antibody, a veneered antibody, a diabody, a humanized antibody, an antibody derivative, a recombinant humanized antibody. In certain embodiments, the equivalent is an aptamer, affimer, non-immunoglobulin scaffold, small molecule, or fragment or derivative thereof.

In certain embodiments, the antibody specifically binds the CD5L monomer. In certain embodiments, the antibody specifically binds the CD5L:CD5L homodimer. In certain embodiments, the antibody specifically binds a CD5L:p40 heterodimer. The antibody can be produced in a suitable cell line.

In certain embodiments, the antagonist is an antibody, an antigen binding fragment or equivalent thereof, small molecule, or genetic modifying agent, said antagonist targeting a downstream target of a CD5L:p40 heterodimer, a CD5L monomer, or a CD5L:CD5L homodimer. The downstream target may be selected from the group consisting of Dusp2, Tmem121, Ppp4c, Vapa, Nubp1, Plk3, Anp32b, Fance, Hccs, Tusc2, Cyth2, Pithd1, Prkca, Nop9, Thap11, Atad3a, Utp18, Marcksl1, Tnfsf11, Nol9, Itsn2, Sumf1, Snx20, Lamp1, Faf1, Gpatch3, Dapk3, 1110065P20Rik, Vaultrc5, Il17f, Il17a, Ildr1, Il1r1, Lgr4, Ptpn14, Paqr8, Timp1, Il1rn, Smim3, Gap43, Tigit, Mmp10, Il22, Enpp2, Iltifb, Ido1, Il23r, Stom, Bcl2l11, 5031414D18Rik, Il24, Itga7, Il6, Epha2, Mt2, Upp1, Snord104, 5730577I03Rik, Slc18b1, Ptprj, Clip3, Mir5104, Ppifos, Rab13, Hist1h2bn, Ass1, Cd200r1, E130112N10Rik, Mxd4, Casp6, Gatm, Tnfrsf8, Gp49a, Gadd45g, Ccr5, Tgm2, Lilrb4, Ecm1, Arhgap18, Serpinb5, Cysltr1, Enpp1, Selp, Slc38a4, Gm14005, Epb4.1l4b, Moxd1, Klra7, Igfbp4, Tnip3, Gstt1, Pglyrp2, Il12rb2, Ctla2a, Plac8, Ly6c1, Sell, Ncf1, Trp53i11, B3gnt3, Kremen2, Matk, Ltb4r1, Ets1, Tnfrsf26, Cd28, Rybp, Ppp1r3c, Thy1, Trib2, Sema3b, Pros1, Il33, Gm5483, Myh11, Cntd1, Ms4a4b, Treml2, 3110009E18Rik, Pglyrp1, Amd1, Slc24a5, Snhg9, Ifi27l1, Irf7, Mx1, Snhg10, Il4, Snora43, H2-L, Myl4, Insl3, Tgoln2, BC022687, C230035Il6Rik, Hvcn1, Myh10, Dhrs3, Acsl6, Rgs2, Ccl20, Ccl3, Dlg2, Ccr6, Ccl4, Duspl4, Apol9b, Cd72, Ispd, Cd70, S100a1, Lgals3, Slc15a3, Nkg7, Serpinc1, Olfr175-ps1, Il9, Pdlim4, Il3, Insl6, Perp, Cd5l, Serpine2, Galntl4, Tff1, Ppfibp2, Bdh2, Mlf1, Il1a, Osr2, Gm5779, Ebf1, Spink2, Egfr and Ccdc155.

In another aspect, the present invention provides for a composition comprising the CD5L antagonist as described herein and a pharmaceutically acceptable carrier. The composition may further comprise an additional active agent used to treat a cancer. The cancer may not be inflammation related. The additional active agent may be one or more checkpoint inhibitors, anti-PD-1, anti-PDL-1, anti-CTLA4, anti-cancer vaccines, adoptive T cell therapy, and/or inhibitory nucleic acids that target CD5L and/or p40. The inhibitory nucleic acids may be genetic modifying agents, small interfering RNAs (e.g., shRNA), antisense oligonucleotides, and/or CRISPR system.

In another aspect, the present invention provides for a method of treating a cancer in a subject comprising administering to the subject a therapeutically effective amount of a CD5L antagonist as described herein or a composition thereof. The method may further comprise sequentially or simultaneously administering an additional active agent used to treat the cancer. The additional active agent may be a standard treatment for the cancer. The cancer treatment may be an immunotherapy treatment. The immunotherapy treatment may be checkpoint blockade therapy. The checkpoint blockade therapy may comprise anti-CTLA4, anti-PD1, anti-PDL1 or combination thereof. The cancer may be adenoid cystic carcinoma (ACC), bladder cancer, breast cancer, cervical cancer, colorectal cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.

In another aspect, the present invention provides for a method for enhancing an immune response in a subject, comprising administering to the subject a therapeutically effective amount of a CD5L antagonist of described herein or a composition thereof. The subject may have an immune deficiency. The immune deficiency may be a primary or secondary immune deficiency. The subject may have an infection with a pathogen. The pathogen may be a viral, bacterial, or fungal pathogen.

In another aspect, the present invention provides for a method of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.

In another aspect, the present invention provides for an antagonistic antibody that associates with an epitope of one or more of a CD5L monomer, a CD5L:CD5L homodimer, and a CD5L:p40 heterodimer.

In another aspect, the present invention provides for a method of screening for an antagonist of one or more of a CD5L monomer, a CD5L:CD5L homodimer, and a CD5L:p40 heterodimer, the method comprising: exposing a cell or a population of cells to an agent that interacts with one or more of a CD5L monomer, a CD5L:CD5L homodimer, and a CD5L:p40 heterodimer; determining expression of a gene or set of genes up and/or down-regulated upon exposure to one or more of a CD5L monomer, a CD5L:CD5L homodimer, a CD5L:p40 heterodimer or antagonist thereof in the cell or population of cells; and determining that the agent is an antagonist based on the gene or set of genes up and/or down-regulated in the cell or population of cells. The antagonist may be an antibody.

In another aspect, the present invention provides for a method of screening for an antagonistic agent comprising: identifying an epitope on one or more of a CD5L monomer, a CD5L:CD5L homodimer, and a CD5L:p40 heterodimer that interacts with an antagonist of one or more of a CD5L monomer, a CD5L:CD5L homodimer, and a CD5L:p40 heterodimer; and screening against a library of candidate antagonistic agents for an antagonistic agent that interacts with the epitope. The antagonist may be an antibody. The antagonistic agent may be an antibody, a small molecule, a peptide, an aptamer, an affimer, a non-immunoglobulin scaffold, or fragment or derivative thereof. The library may comprise a computer database and the screening may comprise a virtual screening. The screening may comprise evaluating the three-dimensional structure of one or more of the CD5L monomer, the CD5L:CD5L homodimer, and the CD5L:p40 heterodimer.

In another aspect, the present invention provides for a method of identifying an agent for treating a cancer that is not inflammation related in a subject, comprising contacting the agent with a T cell, wherein decreased expression of CD5L monomer, CD5L:CD5L homodimer, and/or CD5L:p40 heterodimer indicates that the agent is effective for treating the cancer that is not inflammation related in the subject.

In another aspect, the present invention provides for a method of identifying an agent for enhancing an immune response in a subject, comprising contacting a myeloid cell with the agent, wherein decreased expression of CD5L monomer, CD5L:CD5L homodimer, and/or CD5L:p40 heterodimer indicates that the agent is effective for enhancing the immune response in the subject. The subject may have an immune deficiency. The immune deficiency may be a primary or secondary immune deficiency. The subject may have an infection with a pathogen. The pathogen may be a viral, bacterial, or fungal pathogen.

In another aspect, the present invention provides for a method of treating cancer in a subject, comprising administering to the subject a therapeutically effective amount of a CD5L antagonist as described herein or a composition thereof, wherein the cancer is promoted by complement. The antagonist may be an antibody. The antibody may specifically bind CD5L monomer. The antibody may specifically bind CD5L:CD5L homodimer. The antibody may specifically bind CD5L:p40 heterodimer.

In certain embodiments, the antagonist according to any embodiment herein is an antibody that binds to the fibronectin domain 2 of p40.

Aspects of the disclosure relate to a CD5L monomer, CD5L:CD5L homodimer, and/or CD5L:p40 heterodimer antagonist or and/one or more nucleic acids encoding the same. In some embodiments, the antagonist is an antibody or an antigen binding fragment thereof. In some embodiments, the antagonist is an aptamer, affimer, non-immunoglobulin scaffold, small molecule, or fragment or derivative thereof.

Further aspects of the disclosure relate to methods for enhancing an immune response in a subject, the method comprising administering to the subject a therapeutically effective amount of an antagonist and/or one or more nucleic acids encoding the same. In some embodiments, the subject has cancer, such as a non-inflammation related/non-inflammatory cancer.

Some embodiments comprise administering an anti-cancer immunotherapy to the subject, such as checkpoint inhibitors, PD-1/PDL-1, anti-cancer vaccines, adoptive T cell therapy, and/or combination of two or more thereof.

In some embodiments, the subject has an immune deficiency, e.g., a primary or secondary immune deficiency. In some embodiments, the subject has an infection with a pathogen, e.g., viral, bacterial, or fungal pathogen.

In embodiments that comprise administering inhibitory nucleic acids, the nucleic acids can include small interfering RNAs (e.g., shRNA), antisense oligonucleotides (e.g. antisense RNAs), and/or CRISPR-Cas.

In some embodiments, the subject has an immune deficiency, e.g., a primary or secondary immune deficiency. In some embodiments, the subject has an infection with a pathogen, e.g., viral, bacterial, or fungal pathogen.

Some aspects related to an antagonist against one or more of a CD5L monomer, a CD5L:CD5L homodimer, and a CD5L:p40 heterodimer. In some embodiments, the antagonist is an antibody, or an antigen binding fragment or equivalent thereof, that interacts with (e.g., specifically binds with) one or more of the CD5L monomer, the CD5L:CD5L homodimer, and the CD5L:p40 heterodimer. In some embodiments, the antibody is a polyclonal antibody, a monoclonal antibody, a chimeric antibody, a human antibody, a veneered antibody, a diabody, a humanized antibody, an antibody derivative, a recombinant humanized antibody. In some embodiments, the equivalent is an aptamer, affimer, non-immunoglobulin scaffold, small molecule, or fragment or derivative thereof.

In some embodiments, the antibody specifically binds the CD5L monomer. In some embodiments, the antibody specifically binds the CD5L:CD5L homodimer. In some embodiments, the antibody specifically binds a CD5L:p40 heterodimer. The antibody can be produced in a suitable cell line.

Some aspects relate to compositions comprising an antagonist against one or more of a CD5L monomer, a CD5L:CD5L homodimer, and a CD5L:p40 heterodimer and a pharmaceutically acceptable carrier. Some embodiments further comprise an additional active agent used to treat a cancer that is not inflammation related. In some embodiments, the additional active agent is one or more checkpoint inhibitors, PD-1/PDL-1, anti-cancer vaccines, adoptive T cell therapy, and/or inhibitory nucleic acids that target CD5L and/or p40. In some embodiments, the inhibitory nucleic acids are small interfering RNAs (e.g., shRNA), antisense oligonucleotides, and/or CRISPR-Cas.

Some aspects relate to methods of treating a cancer that is not inflammation related in a subject comprising administering to the subject a therapeutically effective amount of an antagonist against one or more of a CD5L monomer, a CD5L:CD5L homodimer, and a CD5L:p40 heterodimer. Some embodiments further comprise sequentially or simultaneously administering an additional active agent used to treat the cancer. In some embodiments, the additional active agent is a standard treatment for the cancer.

In some embodiments, the cancer is adenoid cystic carcinoma (ACC), bladder cancer, breast cancer, cervical cancer, colorectal cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer.

Some aspects relate to methods for enhancing an immune response in a subject, comprising administering to the subject a therapeutically effective amount of an antagonist against one or more of a CD5L monomer, a CD5L:CD5L homodimer, and a CD5L:p40 heterodimer, or a composition comprising the antagonist. In some embodiments, the subject has an immune deficiency. In some embodiments, the immune deficiency is a primary or secondary immune deficiency.

In some embodiments, the subject has an infection with a pathogen. In some embodiments, the pathogen is a viral, bacterial, or fungal pathogen.

Some aspects relate to methods of modulating CD8⁺ T cell exhaustion in a subject in need thereof, the method comprising administering to the subject a therapeutically effective amount of an antagonist antibody to one or more of a CD5L monomer, a CD5L:CD5L homodimer, and a CD5L:p40 heterodimer.

Some aspects relate to antagonistic antibodies that associate with an epitope of one or more of a CD5L monomer, a CD5L:CD5L homodimer, and a CD5L:p40 heterodimer.

Some aspects relate to methods of identifying a gene or a set of genes up and/or downregulated in response to an agonistic antibody, the method comprising: exposing a cell or population of cells to the antagonist of one or more of a CD5L monomer, a CD5L:CD5L homodimer, and a CD5L:p40 heterodimer, and introducing one or more guide RNAs that target one or more endogenous genes into the cell or population of cells, wherein the cell or population of cells express a CRISPR-Cas9 protein or a CRISPR-Cas9 protein or a nucleic acid encoding the CRISPR-Cas9 protein has been introduced into the cell or population of cells simultaneously or sequentially with the guide RNAs, assaying for a phenotype indicative of enhanced or suppressed immune response, and identifying a gene or set of genes up and/or down regulated in the cell or population of cells with the enhanced or suppressed immune response. In some embodiments, the cell or population of cells are cancer cell(s). In some embodiments, the cancer is adenoid cystic carcinoma (ACC), bladder cancer, breast cancer, cervical cancer, colorectal cancer, ovarian cancer, pheochromocytoma and paraganglioma (PCPG), prostate cancer, uterine Cowden syndrome (CS), uveal melanoma, uterine cancer, head and neck cancer, pancreatic cancer, thyroid cancer, mesothelioma, lung squamous cell (sq) carcinoma, sarcoma, chromophome renal cell carcinoma (chRCC), lung adenocarcinoma, testicular germ cell cancer, cholangiocarcinoma, glioma, papillary renal cell carcinoma (pRCC), glioblastoma (GBM), acute myeloid leukemia (AML), melanoma, clear cell renal cell carcinoma (ccRCC), thymoma, diffuse large B-cell lymphoma (DLBC), or liver cancer. In some embodiments, the cancer cell(s) are human cells. In some embodiments, the human cancer cell(s) have been transplanted into a mouse.

Some aspects relate to methods of treating a cancer that is not inflammation related comprising administering to a subject in need thereof (i) an antagonist of one or more of a CD5L monomer, a CD5L:CD5L homodimer, and a CD5L:p40 heterodimer, and (ii) an agent that targets a gene or set of genes identified as provided herein.

Some aspects relate to methods of screening for an antagonist of one or more of a CD5L monomer, a CD5L:CD5L homodimer, and a CD5L:p40 heterodimer, the method comprising: exposing a cell or a population of cells to an agent that interacts with one or more of a CD5L monomer, a CD5L:CD5L homodimer, and a CD5L:p40 heterodimer; identifying a gene or set of genes up and/or down-regulated in the cell or population of cells; and determining that the agent is an antagonist based on the gene or set of genes up and/or down-regulated in the cell or population of cells. In some embodiments, the antagonist is an antibody. Some embodiments further comprise comparing the identified gene or set of genes to a previously-identified gene or set of genes up and/or down-regulated upon exposure to an antagonist of one or more of a CD5L monomer, a CD5L:CD5L homodimer, and a CD5L:p40 heterodimer.

Some aspects relate to methods of screening for an antagonistic agent comprising: identifying an epitope on one or more of a CD5L monomer, a CD5L:CD5L homodimer, and a CD5L:p40 heterodimer that interacts with an antagonist of one or more of a CD5L monomer, a CD5L:CD5L homodimer, and a CD5L:p40 heterodimer; and screening against a library of candidate antagonistic agents for an antagonistic agent that interacts with the epitope. In some embodiments, the antagonist is an antibody. In some embodiments, the antagonistic agent is an antibody, a small molecule, a peptide, an aptamer, an affimer, a non-immunoglobulin scaffold, or fragment or derivative thereof. In some embodiments, the library comprises a computer database and the screening comprises a virtual screening. In some embodiments, the screening comprises evaluating the three-dimensional structure of one or more of the CD5L monomer, the CD5L:CD5L homodimer, and the CD5L:p40 heterodimer.

Some aspects relate to methods of treating cancer in a subject, comprising administering to the subject a therapeutically effective amount of any of the antagonists described herein or any of the compositions described herein, wherein cancer is promoted by complement.

The invention relates to an antagonist of a CD5L:p40 heterodimer, a CD5L:CD5L homodimer, or a CD5L monomer, wherein the antagonist is capable of inhibiting growth of a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and wherein tumor size is measured up to 14 days or more post-injection.

The antagonist may be capable of increasing the amount of tumor infiltrating CD8⁺ T cells in a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and CD8⁺ tumor infiltrating lymphocytes (TILs) measured up to 14 days or more post-injection.

The antagonist may be capable of increasing the amount of tumor infiltrating CD8⁺ T cells which are positive for interleukin-2 (IL-2) in a MC38 colon carcinoma tumor xenograft in a mouse, e.g. compared to control. The antagonist may be capable of increasing the amount of tumor infiltrating CD8⁺ T cells which are positive for interferon gamma (IFNγ) in a MC38 colon carcinoma tumor xenograft in a mouse, e.g. compared to control. The antagonist may be capable of increasing the amount of tumor infiltrating CD8⁺ T cells which are positive for tumor necrosis factor alpha (TNFα) in a MC38 colon carcinoma tumor xenograft in a mouse, e.g. compared to control. The amount of tumor infiltrating CD8⁺ T cells which are positive for IL-2, IFNγ or TNFα may be assessed following isolation of T cells from the tumor at day 14 and following treatment of T cells with PMA/ionomycin for about 6 hours.

The antagonist may be capable of increasing the amount of tumor infiltrating CD4⁺ T cells in a MC38 colon carcinoma tumor xenograft in a mouse, e.g. compared to control. The MC38 colon carcinoma tumor xenograft may comprise about 1×10⁶ MC38 colon carcinoma cells injected subcutaneously in mice at day 0, and CD8⁺ TILs measured up to 14 days or more post-injection.

The antagonist may be capable of increasing the amount of tumor infiltrating CD4⁺ T cells which are positive for IL-2 in a MC38 colon carcinoma tumor xenograft in a mouse, e.g. compared to control. The antagonist may be capable of increasing the amount of tumor infiltrating CD4⁺ T cells which are positive for TNFα in a MC38 colon carcinoma tumor xenograft in a mouse, e.g. compared to control. The amount of tumor infiltrating CD4⁺ T cells which are positive for IL-2 or TNFα may be assessed following isolation of T cells from the tumor at day 14 and following treatment of T cells with PMA/ionomycin for about 6 hours.

The antagonist may be capable of reducing the amount of myeloid-derived suppressor cells (MDSCs) infiltrating into a MC38 colon carcinoma tumor xenograft in a mouse, e.g. compared to control, including the number of MDSCs which are positive for TNFα. The MC38 colon carcinoma tumor xenograft may comprise about 1×10⁶ MC38 colon carcinoma cells injected subcutaneously in mice at day 0, and MDSCs measured up to 14 days or more post-injection. The amount of infiltrating MDSCs, including the number of MDSCs which are positive for TNFα, may be assessed following isolation of MDSCs from the tumor at day 14 and following treatment of MDSCs with LPS for about 24 hours, with golgi stop/plug added in about the last four hours.

The antagonist may be capable of inhibiting the suppression of the production of IL-17 from pathogenic Th17 (Th17p) cells in vitro mediated by a CD5L:p40 heterodimer or agonist thereof, a CD5L: CD5L homodimer or agonist thereof, or a CD5L monomer or agonist thereof, e.g. compared to control.

The Th17p cells may be differentiated in vitro from naïve T cells under pathogenic Th17 conditions, e.g. using IL-1b, IL-6 and IL-23, and wherein IL-23 may be provided at 0.8 ng/ml or more, 4 ng/ml or more, or 20 ng/ml or more, optionally wherein IL-17 expression is measured in cell supernatant after 3 days of culture. The naïve T cells may be CD44^{low}CD62L⁺CD25-CD4⁺.

The antagonist may be capable of inhibiting the suppression of the production of IFN-γ from Th1 cells in vitro mediated by a CD5L:p40 heterodimer or agonist thereof, a CD5L: CD5L homodimer or agonist thereof, or a CD5L monomer or agonist thereof, e.g. compared to control.

The Th1 cells may be differentiated in vitro from naïve T cells under Th1 conditions, e.g. using IL-12, and wherein IL-12 may be provided at 0.16 ng/ml or more, 0.8 ng/ml or more, 4 ng/ml or more, or 20 ng/ml or more, optionally wherein IFN-γ expression is measured in cell supernatant after 3 days of culture. The naïve T cells may be CD44^{low}CD62L⁺CD25-CD4⁺.

The antagonist may be capable of promoting one or more of IFNγ production from CD8 T cells. The antagonist may be capable of promoting suppression on IL-12 from BMDC-T cells, and/or suppression on IL-23 from BMDC-T cells. The antagonist may be capable of promoting induction of Tim-3, PD-1 or TIGIT expression on T cells from BMDC-T cells coculture. The antagonist may be capable of promoting the induction of MCP-1 from DSS-colitis mouse. The antagonist may inhibit the induction of one or more of Dusp2, Anp32b, 1110065P20Rik, Atad3a, BC022687, Cyth2, Dapk2, Faf1, Fance, Gpatch3, Hccs, Il4, Itsn2, Lamp1, Marcksl1, Nol9, Nop9, Nubp1, Pithd1, Plk3, Ppp4c, Prkca, Snx20, Smnf1, Thap11, Tusc2, and Utp18.

The antagonist may be capable of inhibiting the reduction of neuroinflammation mediated by a CD5L:p40 heterodimer or agonist thereof, a CD5L: CD5L homodimer or agonist thereof, or a CD5L monomer or agonist thereof in a mouse model of experimental autoimmune encephalomyelitis (EAE), e.g. compared to control.

The antagonist may be capable of inhibiting the reduction of the EAE score mediated by a CD5L:p40 heterodimer or agonist thereof, a CD5L: CD5L homodimer or agonist thereof, or a CD5L monomer or agonist thereof in a mouse model of EAE, e.g. compared to control.

Inhibition of the reduction of neuroinflammation and/or EAE score may be observed from 20 days or more following induction of EAE.

The antagonist may be capable of inhibiting the reduction of the amount of CD4 T cells expressing interleukin-17 (IL-17) in CNS mediated by a CD5L:p40 heterodimer or agonist thereof, a CD5L:CD5L homodimer or agonist thereof, or a CD5L monomer or agonist thereof in a mouse model of EAE, e.g. compared to control. Inhibition may be observed from 20 days or more following induction of EAE.

The antagonist may be capable of inhibiting the reduction of the amount of CD4 T cells expressing interferon gamma (IFN-γ) mediated by a CD5L:p40 heterodimer or agonist thereof, a CD5L:CD5L homodimer or agonist thereof, or a CD5L monomer or agonist thereof in a mouse model of EAE, e.g. compared to control. Inhibition may be observed from 20 days or more following induction of EAE.

The mouse model of EAE may comprise immunization of mice with myelin oligodendrocyte glycoprotein (MOG) followed by injection with pertussis toxin (PT) prior to intraperitoneal administration of heterodimer, homodimer, monomer or agonist thereof.

The antagonist may be capable of inhibiting the reduction in colitis mediated by a CD5L:p40 heterodimer or agonist thereof, a CD5L:CD5L homodimer or agonist thereof, or a CD5L monomer or agonist thereof in a mouse model of colitis, e.g. compared to control.

The antagonist may be capable of inhibiting the reduction of weight loss mediated by a CD5L:p40 heterodimer or agonist thereof, a CD5L:CD5L homodimer or agonist thereof in a mouse model of colitis, e.g. compared to control.

Body weight may be measured over a period of 8 days or more following induction of colitis.

The antagonist may be capable of inhibiting the reduction of the amount of CD4 T cells expressing interleukin-17 (IL-17) mediated by a CD5L:p40 heterodimer or agonist thereof, a CD5L:CD5L homodimer or agonist thereof in a mouse model of colitis, e.g. compared to control.

The antagonist may be capable of inhibiting the reduction of the amount of CD4 T cells expressing interferon gamma (IFN-γ) mediated by a CD5L:p40 heterodimer or agonist thereof, a CD5L:CD5L homodimer or agonist thereof in a mouse model of colitis, e.g. compared to control.

The antagonist may be capable of inhibiting the reduction of the amount of group 3 innate lymphoid cells (ILC3s) in colon mediated by a CD5L:p40 heterodimer or agonist thereof, a CD5L:CD5L homodimer or agonist thereof in a mouse model of colitis, e.g. compared to control.

The mouse model of colitis may comprise induction of colitis by administration of 2% dextran sulfate sodium (DSS) in drinking water prior to administration of heterodimer, homodimer, monomer or agonist thereof.

The invention relates to any antagonist of a CD5L:p40 heterodimer, a CD5L:CD5L homodimer, or a CD5L monomer described above and herein for use as a medicament.

The invention relates to the use of any antagonist of a CD5L:p40 heterodimer, a CD5L:CD5L homodimer, or a CD5L monomer described above and herein in the manufacture of a medicament.

The invention relates to a pharmaceutical composition comprising any antagonist of a CD5L:p40 heterodimer, a CD5L:CD5L homodimer, or a CD5L monomer described above and herein and a pharmaceutically acceptable carrier or excipient.

With regard to any of the medical uses, medicaments or pharmaceutical uses, the associated medical treatment may be a method of treating a disease by enhancing the immune response, as described herein. The associated medical treatment may be a method of treating cancer as described herein, such as a non-inflammatory cancer as described herein. The cancer may be any cancer as described herein. The associated medical treatment may be a method of treating a subject that has an immune deficiency, e.g. a primary or secondary immune deficiency as described herein. The associated medical treatment may be a method of treating a subject that has an infection with a pathogen as described herein, e.g. a viral, bacterial or fungal pathogen as described herein. The associated medical treatment may be a method of treating any of the diseases described herein by modulating T cells as described herein.

Any of the CD5L:p40 heterodimer antagonists, CD5L:CD5L homodimer antagonists, CD5L monomer antagonists as described above and herein may be isolated antagonists.

Any of the antagonistic agents described above and herein may be an aptamer, affimer, non-immunoglobulin scaffold, small molecule, or binding portion or fragment or derivative thereof.

Any of the antagonistic agents described above and herein may be an antagonistic antibody or an antagonistic antigen-binding portion, fragment or equivalent thereof as described herein.

An antagonistic agent, such as an antibody or an antagonistic antigen-binding portion, fragment or equivalent thereof, may bind to and antagonize any function of a CD5L:p40 heterodimer, a CD5L:CD5L homodimer and/or a CD5L monomer e.g. as described herein, and wherein the antagonistic agent may possess any of the functional characteristics described above and herein. An antagonistic agent may bind to CD5L, p40, or both CD5L and p40 and antagonize any function of a CD5L:p40 heterodimer. An antagonistic agent may bind to CD5L and antagonize any function of a CD5L:CD5L homodimer or a CD5L monomer. An antagonistic agent may bind to an endogenous CD5L:p40 heterodimer, CD5L:CD5L homodimer and/or CD5L monomer. The antagonistic agent may bind to a recombinant soluble CD5L:p40 heterodimer, CD5L:CD5L homodimer and/or CD5L monomer.

The invention also relates to a cell line producing an antagonistic antibody or an antagonistic antigen-binding portion, fragment or equivalent thereof as described herein. The cell line may be a hybridoma. The cell line may be a transfectoma.

The invention also relates to a nucleic acid molecule encoding an antagonistic antibody or an antagonistic antigen-binding portion, fragment or equivalent thereof as described above and herein.

The invention also relates to any of the methods of screening for an antagonistic agent as described herein, such as an agonistic antibody or an antagonistic antigen-binding portion, fragment or equivalent thereof, wherein the agonistic agent may possess any of the functional characteristics described above and herein.

Any of the antagonistic agents described herein may possess any of the functional characteristics as described above.

With regard to any of the functional characteristics described above, a “control” may be the absence of the heterodimer, homodimer, monomer or agonist as appropriate.

Modified T Cells Having Modulated Function, Activity and/or Expression of Complement Receptor

The invention relates to gene expression signatures and networks of tumors and tissues, as well as multicellular ecosystems of tumors and tissues and the cells and cell type which they comprise. Tumors are multicellular assemblies that encompass many distinct genotypic and phenotypic states. The invention provides for the characterization of components, functions and interactions of tumors and tissues and the cells which they comprise. Single-cell RNA-seq was applied to thousands of malignant and non-malignant cells derived from melanomas, gliomas, head and neck cancer, brain metastases of breast cancer, and tumors in The Cancer Genome Atlas (TCGA) to examine tumor ecosystems. Components of the complement system were found to be correlated to immune cell abundance across different cancer types, however the function of complement in cancer was not elucidated (see e.g., International patent applications PCT/US2016/40015, filed Jun. 29, 2016 and PCT/US2017/014995, filed Jan. 25, 2017; Tirosh et al., Dissecting the multicellular ecosystem of metastatic melanoma by single-cell RNA-seq, Science. 2016 Apr. 8; 352(6282):189-96); Tirosh et al., Single-cell RNA-seq supports a developmental hierarchy in human oligodendroglioma, Nature. (2016), vol. 539, pp. 309-313; and Venteicher et al., Decoupling genetics, lineages, and microenvironment in IDH-mutant gliomas by single-cell RNA-seq, Science. (2017), March 31; 355(6332)), each herein incorporated by reference in its entirety. The present application is based on the discovery of a role for C3 in a tumor control phenotype in vivo. The present invention also provides novel compositions and therapeutic strategies based on modulation of C3 function in the treatment of cancer.

The invention provides signature genes, gene products, and expression profiles of signature genes, gene networks, and gene products of tumors and component cells. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. In one embodiment, the patient is suffering from melanoma. The signature genes, gene products, and expression profiles are useful to identify components of tumors and tissues and states of such components, such as, without limitation, neoplastic cells, malignant cells, stem cells, immune cells, and malignant, microenvironmental, or immunologic states of such component cells.

In one aspect, the present invention provides for an isolated T cell modified to decrease the function, activity and/or expression of complement receptor. The T cell may be a CD8+ T cell, CD4+ T cell, or CD4+ Treg. The complement receptor may be CR1/2 (CD35/CD21). The T cell may express an endogenous T cell receptor (TCR) or chimeric antigen receptor (CAR). The T cell may be a tumor infiltrating lymphocyte (TIL).

In another aspect, the present invention provides for a pharmaceutical composition comprising one or more modified T cells according to any embodiment herein. In certain embodiments, the present invention provides for a method of treating cancer in a subject in need thereof, comprising administering the pharmaceutical composition to the subject. Thus, the present invention provides for obtaining TILs from a subject or for generating a T cell that expresses an endogenous TCR or expresses a CAR and transferring the cells to a subject in need thereof.

In another aspect, the present invention provides for a method of increasing tumor-infiltrating lymphocytes (TILs) in a tumor comprising administering an agent that decreases the activity and/or expression of C3 or a complement receptor.

In another aspect, the present invention provides for a method of treating or enhancing treatment of cancer comprising administering an agent to a subject in need thereof that decreases the activity and/or expression of C3 or a complement receptor.

In certain embodiments, administering of the agent increases an immune response. In certain embodiments, the complement receptor is CR1/2 (CD35/CD21). In certain embodiments, the agent comprises a small molecule, peptide, therapeutic antibody, antibody fragment or antibody-like protein scaffold. In certain embodiments, the agent comprises a CRISPR system, TALE, TALEN, or Zinc Finger protein. In certain embodiments, the agent is an isolated natural product capable of inhibiting C3. In certain embodiments, the agent comprises a metalloproteinase inhibitor, whereby C3 is not cleaved. In certain embodiments, the agent comprises a serine protease inhibitor, whereby C3 is not cleaved. In certain embodiments, administering of the agent decreases lymphangiogenesis. In certain embodiments, administering of the agent decreases PDPN expression in cancer associated fibroblasts (CAFs).

In certain embodiments, the agent is administered in combination with an immunotherapy. The agent may further enhance an immune response. The immunotherapy may be immune checkpoint blockade or adoptive cell therapy. The immune checkpoint blockade may comprise anti-TIM3, anti-CTLA4, anti-PD-L1, anti-PD1, anti-TIGIT, anti-LAG3, or combinations thereof. The adoptive cell therapy may comprise CAR T therapy.

In certain embodiments, the cancer comprises a cancer of the blood, kidney, skin, bone, bladder, colon, brain, breast, head and neck, endometrium, lung, testes, ovary, pancreas or prostate.

In certain embodiments, the method further comprises monitoring efficacy of the treatment comprising detecting PDPN expression in CAFs. In certain embodiments, PDPN expression is decreased if the treatment is effective. Detecting PDPN may be by immunohistochemistry.

### TIL Gene Signatures

In some aspects the T cell has a TIL gene signature. It is an objective of the present invention to identify CD8+ TIL subtypes present in tumor infiltrating lymphocytes (TIL) during tumor growth. It is another objective of the present invention to detect gene signatures and biomarkers specific to the CD8+ TIL subtypes, whereby cells may be detected and isolated. It is another objective of the present invention to provide for adoptive cell transfer methods for treatment of a cancer by transferring more functional CD8+ TIL populations. It is another objective of the present invention to provide for treatment of a cancer by modulating CD8+ T cell populations to be more functional. It is another objective of the present invention to improve immunotherapy treatment.

In one aspect, the present invention provides for an isolated T cell characterized in that the T cell comprises expression of one or more genes selected from the group consisting of: TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2 (Helios), MT1, KIT, SERPINE2, CCRL2, CSF1, EPAS1, RUNX2, SPRY2 and XCR1; or genes in Table 6 of US Pat. App. Pub. 2019/0255107. In another example embodiment, the isolated T cell is characterized in that the T cell does not comprise expression of HMMR and comprises expression of one or more genes selected from TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, MT1, KIT, SERPINE2, CCRL2, CSF1, EPAS1, RUNX2, SPRY2 and XCR1. In another example embodiment, the isolated T cell is characterized by expression of one or more CD8, TIM3, PD1, MT1, and IKZF2, as well as expression of one or more genes selected from the group consisting of TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, KIT, SERPINE2, CCRL2, CSF1, EPAS1, RUNX2, SPRY2 and XCR1. In another example embodiment, the isolated T cell may be characterized by expression of one or more CD8, TIM3, PD1, MT1, and IKZF2, as well as expression of one or more genes selected from the group consisting of TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, KIT, SERPINE2, CCRL2, CSF1, EPAS1, RUNX2, SPRY2 and XCR1, and does not comprise expression of HMMR. The isolated T cell may be further characterized in that the T cell comprises upregulation of one or more genes selected from the group consisting of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2, SPRY2 and XCR1 as compared to all CD8+ TIM3+PD1+ T cells. The isolated T cell may be further characterized in that the T cell comprises downregulation of a cell cycle signature as compared to all CD8+ TIM3+PD1+ T cells. The T cell may be further characterized in that the T cell suppresses T cell proliferation. The isolated T cell may be further characterized by a gene signature comprising one or more genes or polypeptides selected from, preferably, Table 1 of US Pat. App. Pub. 2019/0255107, Table 1 herein, Table 3 of US Pat. App. Pub. 2019/0100801, Table 5 of US Pat. App. Pub. 2019/0255107, and Table 6 of US Pat. App. Pub. 2019/0255107, the genes in ranked order (i.e., most specific to the cells described herein). In certain embodiments, the signature may comprise the top 10, 20, 50, 100, 200, 300, 400, or 500 top genes. In preferred embodiments, the signature comprises genes selected from the top 100, 50, 20, or top 10 genes in each ranked list. In other preferred embodiments, T cells are detected, isolated or targeted using cell surface or cytokines (e.g., Table 3 of US Pat. App. Pub. 2019/0100801). The T cell may be a human cell. The T cell may be autologous for a subject suffering from cancer.

In another aspect, the present invention provides for a method for detecting or quantifying T cells in a biological sample of a subject, the method comprising detecting or quantifying in a biological sample of the subject T cells as defined in any embodiment herein. The T cells may be detected or quantified using a set of markers comprising: a) TIM3, SERPINE2 and HMMR; or b) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2, SPRY2 and XCR1. The T cells may be detected or quantified using a technique selected from the group consisting of RT-PCR, RNA-seq, single cell RNA-seq, flow cytometry, mass cytometry, fluorescence activated cell sorting, fluorescence microscopy, affinity separation, magnetic cell separation, microfluidic separation, and combinations thereof.

In one embodiment, intact T cells may be detected or quantified using a set of surface markers comprising: a) TIM3, SERPINE2 and HMMR; orb) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, IL1R2, SLC2A3, TNFRSF4, KLRC1, IL18R1, TNFRSF18, LAT2, ADAM8, KIT, SERPINE2 and XCR1. The intact T cells may be detected or quantified using a technique selected from the group consisting of flow cytometry, fluorescence activated cell sorting, affinity separation, magnetic cell separation, microfluidic separation, and combinations thereof.

In another aspect, the present invention provides for a method for isolating T cells from a biological sample of a subject, the method comprising isolating from the biological sample T cells as defined in any embodiment herein. The T cells may be isolated using a set of surface markers comprising: a) TIM3, SERPINE2 and HMMR; orb) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, IL1R2, SLC2A3, TNFRSF4, KLRC1, IL18R1, TNFRSF18, LAT2, ADAM8, KIT, SERPINE2 and XCR1. The T cells may be isolated, using a technique selected from the group consisting of flow cytometry, fluorescence activated cell sorting, affinity separation, magnetic cell separation, microfluidic separation, and combinations thereof.

In certain embodiments, the technique for detecting, quantitating, or isolating T cells according to any embodiment herein may employ one or more agents capable of specifically binding to one or more gene products expressed or not expressed by the T cells, preferably on the cell surface of the T cells. The one or more agents may be one or more antibodies.

In certain embodiments, the biological sample may be a tumor sample obtained from a subject. In certain embodiments, the biological sample may be a sample obtained from a subject suffering from an autoimmune disease. In certain embodiments, the biological sample may be a sample obtained from a subject suffering from a chronic infection. Not being bound by a theory, detecting suppressive T cells in a biological sample may provide information as to the immune state of a subject (e.g., for prognosis, treatment selection). In certain embodiments, the biological sample may comprise ex vivo or in vitro T cells. Not being bound by a theory, it may be advantageous to detect or quantitate the presence of suppressive T cells in an ex vivo sample of T cells. For example, after the ex vivo T cells are treated with a differentiating agent or immunomodulatory. Not being bound by a theory, it may be advantageous to deplete suppressive T cells from an ex vivo population of T cells.

In another aspect, the present invention provides for a population of T cells comprising T cells as defined in any embodiment herein. The population of T cells may be depleted for T cells as defined in any embodiment herein by a method of isolation according to any embodiment herein. The population of T cells may comprise chimeric antigen receptor (CAR) T cells or T cells expressing an exogenous T-cell receptor (TCR). The population of T cells may comprise T cells autologous for a subject suffering from cancer. The population of T cells may comprise T cells displaying tumor specificity. Not being bound by a theory, the population of T cells may comprise a heterogeneous population of cells including effector and suppressor T cells. In certain embodiments, it is advantageous to remove the suppressive T cells (e.g., when an enhanced immune response is desired). The population of T cells may be expanded.

In certain embodiments, the population of T cells may comprise activated T cells. The population of T cells may comprise T cells activated with tumor specific antigens. The tumor specific antigens may be subject specific antigens.

In another aspect, the present invention provides for a pharmaceutical composition comprising the depleted T cell population as defined in any embodiment herein.

In another aspect, the present invention provides for a method of treating cancer comprising administering to a subject in need thereof the pharmaceutical composition according to any embodiment herein.

In another aspect, the present invention provides for a method of treating cancer in a subject in need thereof comprising: depleting T cells as defined in any embodiment herein from a population of T cells obtained from the subject; in vitro expanding the population of T cells; and administering the in vitro expanded population of T cells to the subject. The T cell population may be administered after ablation therapy or lymphodepletion therapy. Not being bound by a theory, ablation therapy or lymphodepletion therapy will eliminate any endogenous suppressive cells in a subject, whereby the subject and the cells administered may be depleted for suppressive T cells, thus the adoptive cell therapy may result in an enhanced anti-tumor response.

In another aspect, the present invention provides for a method of treating cancer or chronic infection in a subject in need thereof comprising administering to the subject a therapeutically effective amount of an agent: capable of reducing the activity of a T cell as defined in any embodiment herein; or capable of reducing the activity or expression of one or more genes or polypeptides selected from the group consisting of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2, SPRY2 and XCR1; or capable of targeting or binding to one or more cell surface exposed genes or polypeptides on a T cell as defined in any embodiment herein; or capable of targeting or binding to one or more receptors or ligands specific for a cell surface exposed gene or polypeptide on a T cell as defined in any embodiment herein; or capable of targeting or binding to one or more genes or polypeptides secreted from a T cell as defined in any embodiment herein; or capable of targeting or binding to one or more receptors specific for a gene or polypeptide secreted from a T cell as defined in any embodiment herein. The agent may comprise a therapeutic antibody, antibody fragment, antibody-like protein scaffold, aptamer, protein, CRISPR system or small molecule. The therapeutic antibody may be an antibody drug conjugate. The agent capable of targeting or binding to a cell surface exposed gene or polypeptide may comprise a CAR T cell capable of targeting or binding to the cell surface exposed gene or polypeptide.

In another aspect, the present invention provides for a method of treating an autoimmune disease in a subject in need thereof comprising administering to the subject a therapeutically effective amount of an agent capable of inducing the activity of a T cell as defined in any embodiment herein.

In another aspect, the present invention provides for a method of treating an autoimmune disease comprising administering T cells as defined in any embodiment herein to a subject in need thereof. Not being bound by a theory, administering suppressive T cells may reduce an autoimmune response in a subject.

In another aspect, the present invention provides for a method for identifying an immunomodulant capable of modulating one or more phenotypic aspects of the T cell as defined in any embodiment herein, comprising: applying a candidate immunomodulant to the T cell or T cell population; and detecting modulation of one or more phenotypic aspects of the T cell or T cell population by the candidate immunomodulant, thereby identifying the immunomodulant. The immunomodulant may be capable of modulating suppression of T cell proliferation by the T cell. Thus, in certain embodiments, detecting modulation of one or more phenotypic aspects comprises detecting modulation of a suppressive phenotype. The immunomodulant may comprise a therapeutic antibody, antibody fragment, antibody-like protein scaffold, aptamer, protein or small molecule.

In another aspect, the present invention provides for a pharmaceutical composition comprising the immunomodulant as defined in any embodiment herein.

In another aspect, the present invention provides for a method for determining the T cell status of a subject, or for diagnosing, prognosing or monitoring a disease comprising an immune component in a subject, the method comprising detecting or quantifying in a biological sample of the subject T cells as defined in any embodiment herein, wherein an increase as compared to a reference level indicates a suppressed immune response. The disease may be cancer, an autoimmune disease, or chronic infection.

In another aspect, the present invention provides for a method of preparing cells for use in adoptive cell transfer comprising: obtaining a population of T cells; and depleting suppressive T cells as defined in any embodiment herein from the population of T cells. The method may further comprise expanding the depleted cells. The method may further comprise activating the depleted cells. The population of T cells may comprise CAR T cells. The population of T cells may comprise autologous TILs.

In another aspect, the present invention provides for a method of screening for genes required for suppression of effector T cells by suppressive CD8+ T cells comprising: introducing a library of sgRNAs specific to a set of target genes to a population of T cells expressing a CRISPR system; culturing the cells in proliferating conditions in the presence of suppressive CD8 T cells according to any embodiment herein; determining sgRNAs that are enriched in proliferating T cells.

In another aspect, the present invention provides for a method of treating cancer or chronic infection in a subject in need thereof comprising administering to the subject CD8+ T cells modified to be resistant to suppressive CD8+ T cells, wherein the modified CD8+ T cells may be specific for the cancer or chronic infection. In certain embodiments, the CD8+ T cells modified to be resistant to suppressive CD8+ T cells comprise an inducible suicide gene. Not being bound by a theory, the cells may be killed to prevent a pathogenic autoimmune response.

In another aspect, the present invention provides for a method of treating cancer or chronic infection in a subject in need thereof comprising administering to the subject a therapeutically effective amount of an agent capable of blocking glucocorticoid signaling. The agent may be an antagonist of NR3C1. The antagonist may be a blocking antibody.

In another aspect, the present invention provides for a kit comprising reagents to detect at least one gene or polypeptide as defined in any embodiment herein.

In another aspect, the present invention provides for a method of treating cancer or chronic infection comprising reducing or eliminating the presence of an immune cell or changing a phenotype of the immune cell, at least at a disease or infection loci, wherein the immune cell is characterized by expression of CD8, TIM3, PD1, MT1, and IKZF2, and comprises expression of one or more genes selected from the group consisting of: TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, KIT, SERPINE2, CCRL2, CSF1, EPAS1, RUNX2, SPRY2 and XCR1; or genes in Table 6 of US Pat. App. Pub. 2019/0255107. In certain embodiments, the immune cell does not comprise expression of HMMR. In certain embodiments, the presence of the immune cell is reduced or eliminated, or wherein a phenotype of the immune cell is changed by modulating expression of MT1 and/or MT2. In certain embodiments, the presence of the immune cell is reduced or eliminated, or where a phenotype of the immune cell is changed by modulating expression of HELIOS (IKZF2). In certain embodiments, the presence of the immune cell is reduced or eliminated, or where a phenotype of the immune cell is changed by modulating expression or function of KIT. In certain embodiments, the presence of the immune cell is reduced or eliminated, or where a phenotype of the immune cell is changed by modulating expression or function of SERPINE2. In certain embodiments, the presence of the immune cell is reduced or eliminated, or where a phenotype of the immune cell is changed by modulating expression or function of TNFRSF4. In certain embodiments, the presence of the immune cell is reduced or eliminated, or where a phenotype of the immune cell is changed by modulating expression or function of ILR2. In certain embodiments, the presence of the immune cell is reduced or eliminated, or where a phenotype of the immune cell is changed by modulating expression or function of CSF1. In certain embodiments, the presence of the immune cell is reduced or eliminated, or where a phenotype of the immune cell is changed by modulating expression or function of CCRL2. In certain embodiments, the presence of the immune cell is reduced or eliminated, or where a phenotype of the immune cell is changed by modulating expression or function of IRF8. In certain embodiments, the presence of the immune cell is reduced or eliminated, or where a phenotype of the immune cell is changed, by modulating expression or function of RBPJ. In certain embodiments, the presence of the immune cell is reduced or eliminated, or where a phenotype of the immune cell is changed, by modulating expression or function of EPAS1. In certain embodiments, the presence of the immune cell is reduced or eliminated, or where a phenotype of the immune cell is changed, by modulating expression or function of RUNX2. In certain embodiments, the presence of the immune cell is reduced or eliminated, or where a phenotype of the immune cell is changed, by modulating expression or function of SPRY2. In certain embodiments, the presence of the immune cell is reduced or eliminated, or where a phenotype of the immune cell is changed, by modulating expression or function of STAT3. In certain embodiments, the presence of the immune cell is reduced or eliminated, or wherein a phenotype of the immune cell is changed by modulating expression of XCR1. In certain embodiments, the presence of the immune cell is reduced or eliminated, or where a phenotype of the immune cell is changed, by reducing a sensitivity of the immune cell to glucocorticoid signaling. In certain embodiments, modulating expression or function comprises inhibiting expression or function.

In another aspect, the present invention provides for a kit comprising reagents to detect at least one gene or polypeptide as defined in any of embodiment herein.

In another aspect, the present invention provides for an isolated T cell or population of T cells according to any of the embodiments described herein for use in the manufacture of a medicament for treating cancer, an autoimmune disease or chronic infection.

In another aspect, the present invention provides for a use of a T cell or population of T cells according to any of the embodiments described herein for treating cancer, an autoimmune disease or chronic infection.

In another aspect, the present invention provides for a method of treating cancer or chronic infection in a subject in need thereof comprising administering to the subject a therapeutically effective amount of an agent capable of inhibiting the interaction between XCL1 and XCR1.

An aspect of the invention provides the immune cell or immune cell population as taught herein for use in immunotherapy, such as adoptive immunotherapy, such as adoptive cell transfer. Also provided is a method of treating a subject in need thereof, particularly in need of immunotherapy, such as adoptive immunotherapy, such as adoptive cell transfer, comprising administering to said subject the immune cell or immune cell population as taught herein. Further provided is use of the immune cell or immune cell population as taught herein for the manufacture of a medicament for immunotherapy, such as adoptive immunotherapy, such as adoptive cell transfer. In certain embodiments, the immune cell is a T-cell, such as a CD8+ T-cell. In certain embodiments, the immunotherapy, adoptive immunotherapy or adoptive cell transfer may be for treating a proliferative disease, such as tumor or cancer, or a chronic infection, such as chronic viral infection.

In certain embodiments, an immune cell suitable for immunotherapy, such as a CD8+ T-cell, displays tumor specificity, more particularly displays specificity to a tumor antigen. In certain embodiments, an immune cell suitable for immunotherapy, such as a CD8+ T-cell, displays specificity to an antigen of an infectious agent, for example displays viral antigen specificity. In certain embodiments, an immune cell suitable for immunotherapy, such as a CD8+ T-cell, has been isolated from a tumor of a subject, preferably the cell is a tumor infiltrating lymphocyte (TIL). In certain embodiments, an immune cell suitable for immunotherapy, such as a CD8+ T-cell, comprises a chimeric antigen receptor (CAR). Such cell can also be suitably denoted as having been engineered to comprise or to express the CAR. In certain embodiments, the CAR comprises an extracellular antigen-binding element (or portion or domain) configured to specifically bind to a target antigen, a transmembrane domain, and an intracellular signaling domain. In certain embodiments, the intracellular signaling domain comprises a primary signaling domain and/or a costimulatory signaling domain. In certain embodiments, the CAR comprises the antigen-binding element, costimulatory signaling domain and primary signaling domain (such as CD3 zeta portion) in that order. In certain embodiments, the antigen-binding element comprises, consists of or is derived from an antibody, for example, the antigen-binding element is an antibody fragment. In certain embodiments, the antigen-binding element is derived from, for example is a fragment of, a monoclonal antibody, such as a human monoclonal antibody or a humanized monoclonal antibody. In certain embodiments, the antigen-binding element is a single-chain variable fragment (scFv). In certain preferred embodiments, the target antigen is selected from a group consisting of: CD19, BCMA, CLL-1, MAGE A3, MAGE A6, HPV E6, HPV E7, WT1, CD22, CD171, ROR1, MUC16, and SSX2. In certain preferred embodiments, the target antigen is CD19. In certain embodiments, the transmembrane domain is derived from the most membrane proximal component of the endodomain. In certain embodiments, the transmembrane domain is not CD3 zeta transmembrane domain. In certain embodiments, the transmembrane domain is a CD8α transmembrane domain or a CD28 transmembrane domain, preferably CD28 transmembrane domain. In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain preferred embodiments, the primary signaling domain comprises a functional signaling domain of CD3ζ. In certain embodiments, the one or more costimulatory signaling domains comprise a functional signaling domain of a protein selected, each independently, from the group consisting of: CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain preferred embodiments, the one or more costimulatory signaling domains comprise a functional signaling domain of a protein selected, each independently, from the group consisting of: 4-1BB, CD27, and CD28. In certain preferred embodiments, the costimulatory signaling domain comprises a functional signaling domain of CD28. In certain embodiments, the CAR comprises an anti-CD19 scFv, an intracellular domain of a CD3ζ chain, and a signaling domain of CD28. In certain preferred embodiments, the CD28 sequence is as set forth in Genbank identifier NM_006139 (sequence version 1, 2 or 3) starting with the amino acid sequence IEVMYPPPY (SEQ ID NO:1) and continuing all the way to the carboxy-terminus of the protein. In certain preferred embodiments, the CAR is as included in KTE-C19 (axicabtagene ciloleucel) anti-CD19 CAR-T therapy product in development by Kite Pharma, Inc. In certain embodiments, an immune cell suitable for immunotherapy, such as a CD8+ T-cell, comprises an exogenous T-cell receptor (TCR). Such cell can also be suitably denoted as having been engineered to comprise or to express the TCR.

In certain embodiments, an immune cell suitable for immunotherapy, such as a CD8+ T-cell, may be further genetically modified, such as gene edited, i.e., a target locus of interest in the cell may be modified by a suitable gene editing tool or technique, such as without limitation CRISPR, TALEN or ZFN. An aspect relates to an immune cell obtainable by or obtained by said gene editing method, or progeny thereof, wherein the cell comprises a modification of the target locus not present in a cell not subjected to the method. Another aspect relates to a cell product from said cell or progeny thereof, wherein the product is modified in nature or quantity with respect to a cell product from a cell not subjected to the gene editing method. A further aspect provides an immune cell comprising a gene editing system, such as a CRISPR-Cas system, configured to carry out the modification of the target locus.

In certain preferred embodiments, the cell may be edited using any CRISPR system and method of use thereof as described herein. In certain preferred embodiments, cells are edited ex vivo and transferred to a subject in need thereof.

Further genetically modifying, such as gene editing, of the cell may be performed for example (1) to insert or knock-in an exogenous gene, such as an exogenous gene encoding a CAR or a TCR, at a preselected locus in the cell; (2) to knock-out or knock-down expression of an endogenous TCR in the cell; (3) to disrupt the target of a chemotherapeutic agent in the cell; (4) to knock-out or knock-down expression of an immune checkpoint protein or receptor in the cell; (5) to knock-out or knock-down expression of other gene or genes in the cell, the reduced expression or lack of expression of which can enhance the efficacy of adoptive therapies using the cell; (6) to knock-out or knock-down expression of an endogenous gene in a cell, said endogenous gene encoding an antigen targeted by an exogenous CAR or TCR; (7) to knock-out or knock-down expression of one or more MHC constituent proteins in the cell; (8) to activate a T cell, and/or increase the differentiation and/or proliferation of functionally exhausted or dysfunctional CD8+ T cells; and/or (9) to modulate CD8+ T cells, such that CD8+T cells have increased resistance to exhaustion or dysfunction. In certain preferred embodiments, the cell may be edited to produce any one of the following combinations of the modifications set forth above: (1) and (2); (1) and (4); (2) and (4); (1), (2) and (4); (1) and (7); (2) and (7); (4) and (7); (1), (2) and (7); (1), (4) and (7); (1), (2), (4) and (7); optionally adding modification (8) or (9) to any one of the preceding combinations. In certain preferred embodiments, the targeted immune checkpoint protein or receptor is PD-1, PD-L1 and/or CTLA-4. In certain preferred embodiments, the targeted endogenous TCR gene or sequence may be TRBC1, TRBC2 and/or TRAC. In certain preferred embodiments, the targeted MHC constituent protein may be HLA-A, B and/or C, and/or B2M. In certain embodiments, the cell may thus be multiply edited (multiplex genome editing) to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

It is an objective of the present invention to identify CD8+ TIL subtypes present in tumor infiltrating lymphocytes (TIL) during tumor growth. It is another objective of the present invention to detect gene signatures and biomarkers specific to the CD8+ and/or CD4+ TIL subtypes, whereby cells may be detected and isolated. It is another objective of the present invention to provide for adoptive cell transfer methods for treatment of a cancer by transferring more functional CD8+ and/or CD4+ TIL populations. It is another objective of the present invention to provide for treatment of a cancer by modulating CD8+ and/or CD4+ T cell populations to be more functional. It is another objective of the present invention to improve immunotherapy treatment.

In one aspect, the present invention provides for an isolated CD8⁺ T cell characterized in that the CD8⁺ T cell comprises expression of a gene signature according to any of the following: Table 1 of US Pat. App. Pub. 2019/0255107, Table 1 herein, Table 3 of US Pat. App. Pub. 2019/0100801, Table 5 of US Pat. App. Pub. 2019/0255107, and Table 6 of US Pat. App. Pub. 2019/0255107, The isolated CD8⁺ T cell may be further characterized in that the CD8⁺ expresses HMMR. The isolated CD8⁺ T cell may be further characterized in that the CD8⁺ expresses PD-1 and TIM3. The isolated CD8⁺ T cell may be further characterized in that the CD8⁺ expresses PD-1 and does not express TIM3. The isolated CD8⁺ T cell may be further characterized in that the CD8⁺ expresses PD-1, TIM3, and KI67 and does not express Helios.

In certain embodiments, the CD8⁺ T cell is a human cell. In certain embodiments, the cell is a CAR T cell. In certain embodiments, the cell is a CD8⁺ T cell autologous for a subject suffering from cancer. In certain embodiments, the cell expresses an exogenous CAR or TCR. In certain embodiments, the CD8⁺ T cell displays tumor specificity.

In another aspect, the present invention provides for a method for detecting or quantifying CD8⁺ T cells in a biological sample of a subject, or for isolating CD8⁺ T cells from a biological sample of a subject, the method comprising detecting or quantifying in a biological sample of the subject CD8⁺ T cells as defined herein, or isolating from the biological sample CD8⁺ T cells as defined herein.

In certain embodiments, CD8⁺ T cells are detected, quantified or isolated using one or markers selected from the group consisting of HMMR, PD-1, TIM3, KI67 and Helios.

In certain embodiments, the CD8⁺ T cells are detected, quantified or isolated using a technique selected from the group consisting of flow cytometry, mass cytometry, fluorescence activated cell sorting, fluorescence microscopy, affinity separation, magnetic cell separation, microfluidic separation, and combinations thereof. The technique may employ one or more agents capable of specifically binding to one or more gene products expressed or not expressed by the CD8⁺ T cells, preferably on the cell surface of the CD8⁺ T cells. The one or more agents may be one or more antibodies.

In certain embodiments, the biological sample is a tumor sample obtained from a subject in need thereof and the CD8⁺ T cells are CD8⁺ tumor infiltrating lymphocytes (TIL). The biological sample may comprise ex vivo or in vitro CD8⁺ T cells.

In another aspect, the present invention provides for a population of CD8⁺ T cells comprising CD8⁺ T cells as defined in any embodiment herein or isolated according to any embodiment herein.

In another aspect, the present invention provides for a pharmaceutical composition comprising the CD8⁺ T cell population as defined herein.

In another aspect, the present invention provides for a method for treating or preventing cancer comprising administering to a subject in need thereof the pharmaceutical composition according to any embodiment herein.

In another aspect, the present invention provides for a kit comprising reagents to detect at least one gene or polypeptide as defined herein.

In another aspect, the present invention provides for an isolated T cell characterized in that the T cell comprises expression of one or more genes selected from the group consisting of TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2 (Helios), MT1, KIT, SERPINE2, CCRL2, CSF1, EPAS1, RUNX2 and SPRY2. In another example embodiment, the isolated T cell is characterized in that the T cell does not comprise expression of HMMR and comprises expression of one or more genes selected from TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, MT1, KIT, SERPINE2, CCRL2, CSF1, EPAS1, RUNX2 and SPRY2. In another example embodiment, the isolated T cell is characterized by expression of one or more CD8, TIM3, PD1, MT1, and IKZF2, as well as expression of one or more genes selected from the group consisting of TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, KIT, SERPINE2, CCRL2, CSF1, EPAS1, RUNX2 and SPRY2. In another example embodiment, the isolated T cell may be characterized by expression of one or more CD8, TIM3, PD1, MT1, and IKZF2, as well as expression of one or more genes selected from the group consisting of TNFRSF9, PRF1, BHLHE40 (DEC1), IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, KIT, SERPINE2, CCRL2, CSF1, EPAS1, RUNX2 and SPRY2, and does not comprise expression of HMMR. The isolated T cell may be further characterized in that the T cell comprises upregulation of one or more genes selected from the group consisting of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2 and SPRY2 as compared to all CD8+ TIM3+PD1+ T cells. The isolated T cell may be further characterized in that the T cell comprises downregulation of a cell cycle signature as compared to all CD8+ TIM3+PD1+ T cells. The T cell may be further characterized in that the T cell suppresses T cell proliferation. The isolated T cell may be further characterized by a gene signature comprising one or more genes or polypeptides selected from Table 1 of US Pat. App. Pub. 2019/0255107, Table 1 herein, Table 3 of US Pat. App. Pub. 2019/0100801, and Table 5 of US Pat. App. Pub. 2019/0255107, list the genes in ranked order (i.e., most specific to the cells described herein). In certain embodiments, the signature may comprise the top 10, 20, 50, 100, 200, 300, 400, or 500 top genes. In preferred embodiments, the signature comprises genes selected from the top 100, 50, 20, or top 10 genes in each ranked list. In other preferred embodiments, T cells are detected, isolated or targeted using cell surface or cytokines. (The T cell may be a human cell. The T cell may be autologous for a subject suffering from cancer.

In another aspect, the present invention provides for a method for detecting or quantifying T cells in a biological sample of a subject, the method comprising detecting or quantifying in a biological sample of the subject T cells as defined in any embodiment herein. The T cells may be detected or quantified using a set of markers comprising: a) TIM3, SERPINE2 and HMMR; or b) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2 and SPRY2. The T cells may be detected or quantified using a technique selected from the group consisting of RT-PCR, RNA-seq, single cell RNA-seq, flow cytometry, mass cytometry, fluorescence activated cell sorting, fluorescence microscopy, affinity separation, magnetic cell separation, microfluidic separation, and combinations thereof.

In one embodiment, intact T cells may be detected or quantified using a set of surface markers comprising: a) TIM3, SERPINE2 and HMMR; orb) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, IL1R2, SLC2A3, TNFRSF4, KLRC1, IL18R1, TNFRSF18, LAT2, ADAM8, KIT and SERPINE2. The intact T cells may be detected or quantified using a technique selected from the group consisting of flow cytometry, fluorescence activated cell sorting, affinity separation, magnetic cell separation, microfluidic separation, and combinations thereof.

In another aspect, the present invention provides for a method for isolating T cells from a biological sample of a subject, the method comprising isolating from the biological sample T cells as defined in any embodiment herein. The T cells may be isolated using a set of surface markers comprising: a) TIM3, SERPINE2 and HMMR; orb) SERPINE2 and HMMR; or c) TIM3, KIT and HMMR; or d) TIM3, TNFRSF4 and HMMR; or e) any of (a), (b), (c) or (d) and one or more of CD8, CD45 and PD1; or any of (a), (b), (c), (d) or (e) and one or more of TNFRSF9, IL1R2, SLC2A3, TNFRSF4, KLRC1, IL18R1, TNFRSF18, LAT2, ADAM8, KIT and SERPINE2. The T cells may be isolated, using a technique selected from the group consisting of flow cytometry, fluorescence activated cell sorting, affinity separation, magnetic cell separation, microfluidic separation, and combinations thereof.

In certain embodiments, the technique for detecting, quantitating, or isolating T cells according to any embodiment herein may employ one or more agents capable of specifically binding to one or more gene products expressed or not expressed by the T cells, preferably on the cell surface of the T cells. The one or more agents may be one or more antibodies.

In certain embodiments, the biological sample may be a tumor sample obtained from a subject. In certain embodiments, the biological sample may be a sample obtained from a subject suffering from an autoimmune disease. In certain embodiments, the biological sample may be a sample obtained from a subject suffering from a chronic infection. Not being bound by a theory detecting suppressive T cells in a biological sample may provide information as to the immune state of a subject (e.g., for prognosis, treatment selection). In certain embodiments, the biological sample may comprise ex vivo or in vitro T cells. Not being bound by a theory, it may be advantageous to detect or quantitate the presence of suppressive T cells in an ex vivo sample of T cells. For example, after the ex vivo T cells are treated with a differentiating agent or immunomodulatory. Not being bound by a theory, it may be advantageous to deplete suppressive T cells from an ex vivo population of T cells.

In another aspect, the present invention provides for a population of T cells comprising T cells as defined in any embodiment herein. The population of T cells may be depleted for T cells as defined in any embodiment herein by a method of isolation according to any embodiment herein. The population of T cells may comprise chimeric antigen receptor (CAR) T cells or T cells expressing an exogenous T-cell receptor (TCR). The population of T cells may comprise T cells autologous for a subject suffering from cancer. The population of T cells may comprise T cells displaying tumor specificity. Not being bound by a theory, the population of T cells may comprise a heterogeneous population of cells including effector and suppressor T cells. In certain embodiments, it is advantageous to remove the suppressive T cells (e.g., when an enhanced immune response is desired). The population of T cells may be expanded.

In certain embodiments, the population of T cells may comprise activated T cells. The population of T cells may comprise T cells activated with tumor specific antigens. The tumor specific antigens may be subject specific antigens.

In another aspect, the present invention provides for a pharmaceutical composition comprising the depleted T cell population as defined in any embodiment herein.

In another aspect, the present invention provides for a method of treating cancer comprising administering to a subject in need thereof the pharmaceutical composition according to any embodiment herein.

In another aspect, the present invention provides for a method of treating cancer in a subject in need thereof comprising: depleting T cells as defined in any embodiment herein from a population of T cells obtained from the subject; in vitro expanding the population of T cells; and administering the in vitro expanded population of T cells to the subject. The T cell population may be administered after ablation therapy or lymphodepletion therapy. Not being bound by a theory, ablation therapy or lymphodepletion therapy will eliminate any endogenous suppressive cells in a subject, whereby the subject and the cells administered may be depleted for suppressive T cells, thus the adoptive cell therapy may result in an enhanced anti-tumor response.

In another aspect, the present invention provides for a method of treating cancer or chronic infection in a subject in need thereof comprising administering to the subject a therapeutically effective amount of an agent: capable of reducing the activity of a T cell as defined in any embodiment herein; or capable of reducing the activity or expression of one or more genes or polypeptides selected from the group consisting of TNFRSF9, PRF1, BHLHE40, IRF8, GLDC, STAT3, CST7, IL1R2, EEF2, SLC2A3, SQSTM1, RBPJ, NABP1, ACTN1, TNFRSF4, SERPINB9, FOSL2, CAPG, KLRC1, IL18R1, JUNB, EEF1A1, TNFRSF18, RGS2, NFKB2, RPL5, PEX16, LAT2, KDM5B, HILPDA, GEM, DENND4A, BCL2L11, ADAM8, PGLYRP1, IKZF2, KIT, SERPINE2, CCRL2, CSF1, EPAS1, RUNX2 and SPRY2; or capable of targeting or binding to one or more cell surface exposed genes or polypeptides on a T cell as defined in any embodiment herein; or capable of targeting or binding to one or more receptors or ligands specific for a cell surface exposed gene or polypeptide on a T cell as defined in any embodiment herein; or capable of targeting or binding to one or more genes or polypeptides secreted from a T cell as defined in any embodiment herein; or capable of targeting or binding to one or more receptors specific for a gene or polypeptide secreted from a T cell as defined in any embodiment herein. The agent may comprise a therapeutic antibody, antibody fragment, antibody-like protein scaffold, aptamer, protein, CRISPR system or small molecule. The therapeutic antibody may be an antibody drug conjugate. The agent capable of targeting or binding to a cell surface exposed gene or polypeptide may comprise a CAR T cell capable of targeting or binding to the cell surface exposed gene or polypeptide.

In another aspect, the present invention provides for a method of treating an autoimmune disease in a subject in need thereof comprising administering to the subject a therapeutically effective amount of an agent capable of inducing the activity of a T cell as defined in any embodiment herein.

In another aspect, the present invention provides for a method of treating an autoimmune disease comprising administering T cells as defined in any embodiment herein to a subject in need thereof. Not being bound by a theory, administering suppressive T cells may reduce an autoimmune response in a subject.

In another aspect, the present invention provides for a method for identifying an immunomodulant capable of modulating one or more phenotypic aspects of the T cell as defined in any embodiment herein, comprising: applying a candidate immunomodulant to the T cell or T cell population; and detecting modulation of one or more phenotypic aspects of the T cell or T cell population by the candidate immunomodulant, thereby identifying the immunomodulant. The immunomodulant may be capable of modulating suppression of T cell proliferation by the T cell. Thus, in certain embodiments, detecting modulation of one or more phenotypic aspects comprises detecting modulation of a suppressive phenotype. The immunomodulant may comprise a therapeutic antibody, antibody fragment, antibody-like protein scaffold, aptamer, protein or small molecule.

In another aspect, the present invention provides for a pharmaceutical composition comprising the immunomodulant as defined in any embodiment herein.

In another aspect, the present invention provides for a method for determining the T cell status of a subject, or for diagnosing, prognosing or monitoring a disease comprising an immune component in a subject, the method comprising detecting or quantifying in a biological sample of the subject T cells as defined in any embodiment herein, wherein an increase as compared to a reference level indicates a suppressed immune response. The disease may be cancer, an autoimmune disease, or chronic infection.

In another aspect, the present invention provides for a method of preparing cells for use in adoptive cell transfer comprising: obtaining a population of T cells; and depleting suppressive T cells as defined in any embodiment herein from the population of T cells. The method may further comprise expanding the depleted cells. The method may further comprise activating the depleted cells. The population of T cells may comprise CAR T cells. The population of T cells may comprise autologous TILs.

In another aspect, the present invention provides for a method of screening for genes required for suppression of effector T cells by suppressive CD8+ T cells comprising: introducing a library of sgRNAs specific to a set of target genes to a population of T cells expressing a CRISPR system; culturing the cells in proliferating conditions in the presence of suppressive CD8 T cells according to any embodiment herein; determining sgRNAs that are enriched in proliferating T cells.

In another aspect, the present invention provides for a method of treating cancer or chronic infection in a subject in need thereof comprising administering to the subject CD8+ T cells modified to be resistant to suppressive CD8+ T cells, wherein the modified CD8+ T cells may be specific for the cancer or chronic infection. In certain embodiments, the CD8+ T cells modified to be resistant to suppressive CD8+ T cells comprise an inducible suicide gene. Not being bound by a theory, the cells may be killed to prevent a pathogenic autoimmune response.

In another aspect, the present invention provides for a method of treating cancer or chronic infection in a subject in need thereof comprising administering to the subject a therapeutically effective amount of an agent capable of blocking glucocorticoid signaling. The agent may be an antagonist of NR3C1. The antagonist may be a blocking antibody.

In another aspect, the present invention provides for a kit comprising reagents to detect at least one gene or polypeptide as defined in any embodiment herein.

An aspect of the invention provides the immune cell or immune cell population as taught herein for use in immunotherapy, such as adoptive immunotherapy, such as adoptive cell transfer. Also provided is a method of treating a subject in need thereof, particularly in need of immunotherapy, such as adoptive immunotherapy, such as adoptive cell transfer, comprising administering to said subject the immune cell or immune cell population as taught herein. Further provided is use of the immune cell or immune cell population as taught herein for the manufacture of a medicament for immunotherapy, such as adoptive immunotherapy, such as adoptive cell transfer. In certain embodiments, the immune cell is a T-cell, such as a CD8+ T-cell. In certain embodiments, the immunotherapy, adoptive immunotherapy or adoptive cell transfer may be for treating a proliferative disease, such as tumor or cancer, or a chronic infection, such as chronic viral infection.

In certain embodiments, an immune cell suitable for immunotherapy, such as a CD8+ T-cell, displays tumor specificity, more particularly displays specificity to a tumor antigen. In certain embodiments, an immune cell suitable for immunotherapy, such as a CD8+T-cell, displays specificity to an antigen of an infectious agent, for example displays viral antigen specificity. In certain embodiments, an immune cell suitable for immunotherapy, such as a CD8+ T-cell, has been isolated from a tumor of a subject, preferably the cell is a tumor infiltrating lymphocyte (TIL). In certain embodiments, an immune cell suitable for immunotherapy, such as a CD8+ T-cell, comprises a chimeric antigen receptor (CAR). Such cell can also be suitably denoted as having been engineered to comprise or to express the CAR. In certain embodiments, the CAR comprises an extracellular antigen-binding element (or portion or domain) configured to specifically bind to a target antigen, a transmembrane domain, and an intracellular signaling domain. In certain embodiments, the intracellular signaling domain comprises a primary signaling domain and/or a costimulatory signaling domain. In certain embodiments, the CAR comprises the antigen-binding element, costimulatory signaling domain and primary signaling domain (such as CD3 zeta portion) in that order. In certain embodiments, the antigen-binding element comprises, consists of or is derived from an antibody, for example, the antigen-binding element is an antibody fragment. In certain embodiments, the antigen-binding element is derived from, for example is a fragment of, a monoclonal antibody, such as a human monoclonal antibody or a humanized monoclonal antibody. In certain embodiments, the antigen-binding element is a single-chain variable fragment (scFv). In certain preferred embodiments, the target antigen is selected from a group consisting of: CD19, BCMA, CLL-1, MAGE A3, MAGE A6, HPV E6, HPV E7, WT1, CD22, CD171, ROR1, MUC16, and SSX2. In certain preferred embodiments, the target antigen is CD19. In certain embodiments, the transmembrane domain is derived from the most membrane proximal component of the endodomain. In certain embodiments, the transmembrane domain is not CD3 zeta transmembrane domain. In certain embodiments, the transmembrane domain is a CD8α transmembrane domain or a CD28 transmembrane domain, preferably CD28 transmembrane domain. In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain preferred embodiments, the primary signaling domain comprises a functional signaling domain of CD3ζ. In certain embodiments, the one or more costimulatory signaling domains comprise a functional signaling domain of a protein selected, each independently, from the group consisting of: CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain preferred embodiments, the one or more costimulatory signaling domains comprise a functional signaling domain of a protein selected, each independently, from the group consisting of: 4-1BB, CD27, and CD28. In certain preferred embodiments, the costimulatory signaling domain comprises a functional signaling domain of CD28. In certain embodiments, the CAR comprises an anti-CD19 scFv, an intracellular domain of a CD3ζ chain, and a signaling domain of CD28. In certain preferred embodiments, the CD28 sequence is as set forth in Genbank identifier NM_006139 (sequence version 1, 2 or 3) starting with the amino acid sequence IEVMYPPPY and continuing all the way to the carboxy-terminus of the protein. In certain preferred embodiments, the CAR is as included in KTE-C19 (axicabtagene ciloleucel) anti-CD19 CAR-T therapy product in development by Kite Pharma, Inc. In certain embodiments, an immune cell suitable for immunotherapy, such as a CD8+ T-cell, comprises an exogenous T-cell receptor (TCR). Such cell can also be suitably denoted as having been engineered to comprise or to express the TCR.

In certain embodiments, an immune cell suitable for immunotherapy, such as a CD8+ T-cell, may be further genetically modified, such as gene edited, i.e., a target locus of interest in the cell may be modified by a suitable gene editing tool or technique, such as without limitation CRISPR, TALEN or ZFN. An aspect relates to an immune cell obtainable by or obtained by said gene editing method, or progeny thereof, wherein the cell comprises a modification of the target locus not present in a cell not subjected to the method. Another aspect relates to a cell product from said cell or progeny thereof, wherein the product is modified in nature or quantity with respect to a cell product from a cell not subjected to the gene editing method. A further aspect provides an immune cell comprising a gene editing system, such as a CRISPR-Cas system, configured to carry out the modification of the target locus.

In certain preferred embodiments, the cell may be edited using any CRISPR system and method of use thereof as described herein. In certain preferred embodiments, cells are edited ex vivo and transferred to a subject in need thereof.

Further genetically modifying, such as gene editing, of the cell may be performed for example (1) to insert or knock-in an exogenous gene, such as an exogenous gene encoding a CAR or a TCR, at a preselected locus in the cell; (2) to knock-out or knock-down expression of an endogenous TCR in the cell; (3) to disrupt the target of a chemotherapeutic agent in the cell; (4) to knock-out or knock-down expression of an immune checkpoint protein or receptor in the cell; (5) to knock-out or knock-down expression of other gene or genes in the cell, the reduced expression or lack of expression of which can enhance the efficacy of adoptive therapies using the cell; (6) to knock-out or knock-down expression of an endogenous gene in a cell, said endogenous gene encoding an antigen targeted by an exogenous CAR or TCR; (7) to knock-out or knock-down expression of one or more MHC constituent proteins in the cell; (8) to activate a T cell, and/or increase the differentiation and/or proliferation of functionally exhausted or dysfunctional CD8+ T cells; and/or (9) to modulate CD8+ T cells, such that CD8+ T cells have increased resistance to exhaustion or dysfunction. In certain preferred embodiments, the cell may be edited to produce any one of the following combinations of the modifications set forth above: (1) and (2); (1) and (4); (2) and (4); (1), (2) and (4); (1) and (7); (2) and (7); (4) and (7); (1), (2) and (7); (1), (4) and (7); (1), (2), (4) and (7); optionally adding modification (8) or (9) to any one of the preceding combinations. In certain preferred embodiments, the targeted immune checkpoint protein or receptor is PD-1, PD-L1 and/or CTLA-4. In certain preferred embodiments, the targeted endogenous TCR gene or sequence may be TRBC1, TRBC2 and/or TRAC. In certain preferred embodiments, the targeted MHC constituent protein may be HLA-A, B and/or C, and/or B2M. In certain embodiments, the cell may thus be multiply edited (multiplex genome editing) to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

In one aspect, the invention provides a method of treating cancer in a subject in need thereof comprising administering an agent capable of blocking the interaction of KLRB1 with its ligand. In an exemplary aspect, the KLRB1 ligand is CLEC2D. In some embodiments, the agent is a soluble KLRB1 protein or fragment thereof. The agent may comprise an antibody or fragment thereof. The antibody may be a humanized or chimeric antibody. In some embodiments, the antibody binds KLRB1. In alternative embodiments, the antibody binds CLEC2D. The agent may be a programmable nucleic acid modifying agent. The programmable nucleic acid modifying agent may be a CRISPR-Cas system, a zinc finger system, a TALE system, or a meganuclease. In specific embodiments, the CRISPR-Cas system may be a CRISPR-Cas9 system, a CRISPR-Cpf1 system, or a CRISPR-Cas13 system. The agent may be administered in a combination treatment regimen such as checkpoint blockade therapy and/or adoptive cell therapy (ACT). In certain embodiments, the checkpoint blockade therapy includes, but is not limited to, anti-PD-1, anti-CTLA4, anti-PDL1, anti-TIM-3 and/or anti-LAG3. Alternatively, the agent may be administered in a combination treatment regimen that includes a neoantigen vaccine. In some embodiments, the cancer may express CLEC2D. In some embodiments, immune cells in the tumor microenvironment express KLRB1. In some embodiments, the immune cells are tumor infiltrating lymphocytes. In example embodiments, the cancer may include, but is not limited to, glioblastoma (GMB), renal cancer, lung adenocarcinoma, or colonadenocarcinoma.

In another aspect, the invention provides an isolated T cell or a population or T cells that is modified to have decreased expression or activity of, or is modified to have an agent capable of decreasing expression or activity of KLRB1. In some embodiments, the T cell or population thereof is CD8+. Alternatively, the T cell or population thereof may be CD4+. The T cell or population thereof may be obtained from peripheral blood mononuclear cells (PBMCs). The T cell or population thereof may be autologous T cell(s) from a subject in need thereof. The T cell or population thereof may be tumor infiltrating leukocyte(s) (TIL) obtained from a subject in need thereof. The T cell or population thereof may have a chimeric antigen receptor (CAR) or an exogenous T-cell receptor (TCR). In some embodiments, the exogenous TCR may be clonally expanded in a tumor. In certain embodiments, the CAR or TCR may be specific for a tumor antigen. In some embodiments, the tumor antigen may be EGFRvIII. In other exemplary embodiments, the tumor antigen may be selected from the group consisting of: B cell maturation antigen (BCMA); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); and any combination thereof.

In certain embodiments, the T cell or population thereof is further modified to have decreased expression or activity of, or is modified to have an agent capable of decreasing expression or activity of a gene or polypeptide selected from the group consisting of TOB1, RGS 1, TARP, NKG7, CCL4 and any combination thereof. In some embodiments, the T cell or population thereof may be activated. In some embodiments, the T cell or population thereof is modified using a CRISPR system having guide sequences specific to the target. The CRISPR system may include, but is not limited to, Cas9, Cpf1 or Cas13.

In yet another aspect, the invention provides a pharmaceutical composition comprising the population of isolated T cells described herein.

In yet another aspect, the invention provides a method of treating cancer in a subject in need thereof by administering the pharmaceutical composition described herein. In some embodiments, the population of cells is administered by infusion into the cerebral spinal fluid (CSF). In specific embodiments, the population of cells is administered by injection into the CSF through the lateral ventricle. In other embodiments, the population of cells is administered in a combination treatment regimen comprising checkpoint blockade therapy. In specific exemplary embodiments, the checkpoint blockade therapy may include anti-PD-1, anti-CTLA4, anti-PDL1, anti-TIM-3 and/or anti-LAG3. In some embodiments, the cancer may express CLEC2D. Tumor infiltrating lymphocytes (TILs) in the cancer may express KLRB1. In some forms, the cancer may be glioblastoma (GBM).

In yet another aspect, the invention provides a method of generating a population of T cells for adoptive cell transfer. The method may include the steps of a) obtaining a population of T cells; b) delivering to the population of T cells a CRISPR system having one or more guide sequences targeting KLRB1; and c) activating the population of cells. In some embodiments, the CRISPR system may include Cas9, Cpf1 or Cas13. In some embodiments, the CRISPR system is delivered as a ribonucleoprotein (RNP) complex by electroporation. In some embodiments, activating involves culturing the populations of cells with αCD3 and αCD28 beads and IL-2. The method may further involve transducing the population of cells with a vector encoding a chimeric antigen receptor (CAR) or an exogenous T-cell receptor (TCR). Optionally, the vector further encodes a detectable marker and the T cells expressing a CAR or TCR are purified by sorting cells positive for the detectable marker. In some embodiments, the T cells may be obtained from TILs obtained from a subject in need of treatment. The T cells may be obtained from PBMCs, and the PBMCs may be obtained from a subject in need of treatment.

In yet another aspect, the invention provides a soluble KLRB1 protein or fragment thereof for use in the treatment of cancer.

In yet another aspect, the invention provides a KLRB1 antibody for use in the treatment of cancer.

In yet another aspect, the invention provides a CLEC2D antibody for use in the treatment of cancer.

### Modulating T Cell Balance

The invention provides compositions and methods for modulating T cell balance, e.g., Th17 cell differentiation, maintenance and function, and means for exploiting this network in a variety of therapeutic and diagnostic methods. As used herein, the term “modulating” includes up-regulation of, or otherwise increasing, the expression of one or more genes, down-regulation of, or otherwise decreasing, the expression of one or more genes, inhibiting or otherwise decreasing the expression, activity and/or function of one or more gene products, and/or enhancing or otherwise increasing the expression, activity and/or function of one or more gene products.

As used herein, the term “modulating T cell balance” includes the modulation of any of a variety of T cell-related functions and/or activities, including by way of non-limiting example, controlling or otherwise influencing the networks that regulate T cell differentiation; controlling or otherwise influencing the networks that regulate T cell maintenance, for example, over the lifespan of a T cell; controlling or otherwise influencing the networks that regulate T cell function; controlling or otherwise influencing the networks that regulate helper T cell (Th cell) differentiation; controlling or otherwise influencing the networks that regulate Th cell maintenance, for example, over the lifespan of a Th cell; controlling or otherwise influencing the networks that regulate Th cell function; controlling or otherwise influencing the networks that regulate Th17 cell differentiation; controlling or otherwise influencing the networks that regulate Th17 cell maintenance, for example, over the lifespan of a Th17 cell; controlling or otherwise influencing the networks that regulate Th17 cell function; controlling or otherwise influencing the networks that regulate regulatory T cell (Treg) differentiation; controlling or otherwise influencing the networks that regulate Treg cell maintenance, for example, over the lifespan of a Treg cell; controlling or otherwise influencing the networks that regulate Treg cell function; controlling or otherwise influencing the networks that regulate other CD4+ T cell differentiation; controlling or otherwise influencing the networks that regulate other CD4+ T cell maintenance; controlling or otherwise influencing the networks that regulate other CD4+ T cell function; manipulating or otherwise influencing the ratio of T cells such as, for example, manipulating or otherwise influencing the ratio of Th17 cells to other T cell types such as Tregs or other CD4+ T cells; manipulating or otherwise influencing the ratio of different types of Th17 cells such as, for example, pathogenic Th17 cells and non-pathogenic Th17 cells; manipulating or otherwise influencing at least one function or biological activity of a T cell; manipulating or otherwise influencing at least one function or biological activity of Th cell; manipulating or otherwise influencing at least one function or biological activity of a Treg cell; manipulating or otherwise influencing at least one function or biological activity of a Th17 cell; and/or manipulating or otherwise influencing at least one function or biological activity of another CD4+ T cell.

The invention provides T cell modulating agents that modulate T cell balance. For example, in some embodiments, the invention provides T cell modulating agents and methods of using these T cell modulating agents to regulate, influence or otherwise impact the level(s) of and/or balance between T cell types, e.g., between Th17 and other T cell types, for example, regulatory T cells (Tregs), and/or Th17 activity and inflammatory potential. As used herein, terms such as “Th17 cell” and/or “Th17 phenotype” and all grammatical variations thereof refer to a differentiated T helper cell that expresses one or more cytokines selected from the group consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

For example, in some embodiments, the invention provides T cell modulating agents and methods of using these T cell modulating agents to regulate, influence or otherwise impact the level of and/or balance between Th17 phenotypes, and/or Th17 activity and inflammatory potential. Suitable T cell modulating agents include an antibody, a soluble polypeptide, a polypeptide agent, a peptide agent, a nucleic acid agent, a nucleic acid ligand, or a small molecule agent.

For example, in some embodiments, the invention provides T cell modulating agents and methods of using these T cell modulating agents to regulate, influence or otherwise impact the level of and/or balance between Th17 cell types, e.g., between pathogenic and non-pathogenic Th17 cells. For example, in some embodiments, the invention provides T cell modulating agents and methods of using these T cell modulating agents to regulate, influence or otherwise impact the level of and/or balance between pathogenic and non-pathogenic Th17 activity.

For example, in some embodiments, the invention provides T cell modulating agents and methods of using these T cell modulating agents to influence or otherwise impact the differentiation of a population of T cells, for example toward Th17 cells, with or without a specific pathogenic distinction, or away from Th17 cells, with or without a specific pathogenic distinction.

For example, in some embodiments, the invention provides T cell modulating agents and methods of using these T cell modulating agents to influence or otherwise impact the differentiation of a population of T cells, for example toward a non-Th17 T cell subset or away from a non-Th17 cell subset. For example, in some embodiments, the invention provides T cell modulating agents and methods of using these T cell modulating agents to induce T-cell plasticity, i.e., converting Th17 cells into a different subtype, or into a new state.

For example, in some embodiments, the invention provides T cell modulating agents and methods of using these T cell modulating agents to induce T cell plasticity, e.g., converting Th17 cells into a different subtype, or into a new state.

For example, in some embodiments, the invention provides T cell modulating agents and methods of using these T cell modulating agents to achieve any combination of the above.

In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

The T cell modulating agents are used to modulate the expression of one or more target genes or one or more products of one or more target genes that have been identified as genes responsive to Th17-related perturbations. These target genes are identified, for example, contacting a T cell, e.g., naïve T cells, partially differentiated T cells, differentiated T cells and/or combinations thereof, with a T cell modulating agent and monitoring the effect, if any, on the expression of one or more signature genes or one or more products of one or more signature genes. In some embodiments, the one or more signature genes are selected from those listed in Table 1 of US Pat. App. Pub. 2019/0255107 or Table 2 herein.

In some embodiments, the target gene is one or more Th17-associated cytokine(s) or receptor molecule(s).

In some embodiments, the target gene is one or more Th17-associated transcription regulator(s) selected from those shown in Table 5 of US Pat. App. Pub. 2019/0255107 of the specification. In some embodiments, the target gene is one or more Th17-associated receptor molecule(s) selected from those listed in Table 6 of US Pat. App. Pub. 2019/0255107 of the specification. In some embodiments, the target gene is one or more Th17-associated kinase(s) selected from those listed in Table 7 of US Pat. App. Pub. 2019/0255107 of the specification. In some embodiments, the target gene is one or more Th17-associated signaling molecule(s) selected from those listed in Table 8 of US Pat. App. Pub. 2019/0255107 of the specification. In some embodiments, the target gene is one or more Th17-associated receptor molecule(s) selected from those listed in Table 9 of US Pat. App. Pub. 2019/0255107 of the specification.

In some embodiments, the target gene is one or more target genes involved in induction of Th17 differentiation such as, for example, IRF1, IRF8, IRF9, STAT2, STAT3, IRF7, STAT1, ZFP281, IFI35, REL, TBX21, FLI1, BATF, IRF4, one or more of the target genes listed in Table 5 of US Pat. App. Pub. 2019/0255107 as being associated with the early stage of Th17 differentiation, maintenance and/or function, e.g., AES, AHR, ARID5A, BATF, BCL11B, BCL3, CBFB, CBX4, CHD7, CITED2, CREB1, E2F4, EGR1, EGR2, ELL2, ETS1, ETS2, ETV6, EZH1, FLI1, FOXO1, GATA3, GATAD2B, HIF1A, ID2, IFI35, IKZF4, IRF1, IRF2, IRF3, IRF4, IRF7, IRF9, JMJD1C, JUN, LEF1, LRRFIP1, MAX, NCOA3, NFE2L2, NFIL3, NFKB1, NMI, NOTCH1, NR3C1, PHF21A, PML, PRDM1, REL, RELA, RUNX1, SAP18, SATB1, SMAD2, SMARCA4, SP100, SP4, STAT1, STAT2, STAT3, STAT4, STAT5B, STAT6, TFEB, TP53, TRIM24, and/or ZFP161, or any combination thereof.

In some embodiments, the target gene is one or more target genes involved in onset of Th17 phenotype and amplification of Th17 T cells such as, for example, IRF8, STAT2, STAT3, IRF7, JUN, STAT5B, ZPF2981, CHD7, TBX21, FLI1, SATB1, RUNX1, BATF, RORC, SP4, one or more of the target genes listed in Table 5 of US Pat. App. Pub. 2019/0255107 as being associated with the intermediate stage of Th17 differentiation, maintenance and/or function, e.g., AES, AHR, ARID3A, ARID5A, ARNTL, ASXL1, BATF, BCL11B, BCL3, BCL6, CBFB, CBX4, CDC5L, CEBPB, CHD7, CREB1, CREB3L2, CREM, E2F4, E2F8, EGR1, EGR2, ELK3, ELL2, ETS1, ETS2, ETV6, EZH1, FLI1, FOSL2, FOXJ2, FOXO1, FUS, HIF1A, HMGB2, ID1, ID2, IFI35, IKZF4, IRF3, IRF4, IRF7, IRF8, IRF9, JUN, JUNB, KAT2B, KLF10, KLF6, KLF9, LEF1, LRRFIP1, MAFF, MAX, MAZ, MINA, MTA3, MYC, MYST4, NCOA1, NCOA3, NFE2L2, NFIL3, NFKB1, NMI, NOTCH1, NR3C1, PHF21A, PML, POU2AF1, POU2F2, PRDM1, RARA, RBPJ, RELA, RORA, RUNX1, SAP18, SATB1, SKI, SKIL, SMAD2, SMAD7, SMARCA4, SMOX, SP1, SP4, SS18, STAT1, STAT2, STAT3, STAT5A, STAT5B, STAT6, SUZ12, TBX21, TFEB, TLE1, TP53, TRIM24, TRIM28, TRPS1, VAV1, ZEB1, ZEB2, ZFP161, ZFP62, ZNF238, ZNF281, and/or ZNF703, or any combination thereof.

In some embodiments, the target gene is one or more target genes involved in stabilization of Th17 cells and/or modulating Th17-associated interleukin 23 (IL-23) signaling such as, for example, STAT2, STAT3, JUN, STAT5B, CHD7, SATB1, RUNX1, BATF, RORC, SP4 IRF4, one or more of the target genes listed in Table 5 of US Pat. App. Pub. 2019/0255107 as being associated with the late stage of Th17 differentiation, maintenance and/or function, e.g., AES, AHR, ARID3A, ARID5A, ARNTL, ASXL1, ATF3, ATF4, BATF, BATF3, BCL11B, BCL3, BCL6, C210RF66, CBFB, CBX4, CDC5L, CDYL, CEBPB, CHD7, CHMP1B, CIC, CITED2, CREB1, CREB3L2, CREM, CSDA, DDIT3, E2F1, E2F4, E2F8, EGR1, EGR2, ELK3, ELL2, ETS1, ETS2, EZH1, FLI1, FOSL2, FOXJ2, FOXO1, FUS, GATA3, GATAD2B, HCLS1, HIF1A, ID1, ID2, IFI35, IKZF4, IRF3, IRF4, IRF7, IRF8, IRF9, JARID2, JMJD1C, JUN, JUNB, KAT2B, KLF10, KLF6, KLF7, KLF9, LASS4, LEF1, LRRFIP1, MAFF, MAX, MEN1, MINA, MTA3, MXI1, MYC, MYST4, NCOA1, NCOA3, NFE2L2, NFIL3, NFKB1, NMI, NOTCH1, NR3C1, PHF13, PHF21A, PML, POU2AF1, POU2F2, PRDM1, RARA, RBPJ, REL, RELA, RNF11, RORA, RORC, RUNX1, RUNX2, SAP18, SAP30, SATB1, SERTAD1, SIRT2, SKI, SKIL, SMAD2, SMAD4, SMAD7, SMARCA4, SMOX, SP1, SP100, SP4, SS18, STAT1, STAT3, STAT4, STAT5A, STAT5B, STAT6, SUZ12, TBX21, TFEB, TGIF1, TLE1, TP53, TRIM24, TRPS1, TSC22D3, UBE2B, VAV1, VAX2, XBP1, ZEB1, ZEB2, ZFP161, ZFP36L1, ZFP36L2, ZNF238, ZNF281, ZNF703, ZNRF1, and/or ZNRF2, or any combination thereof.

In some embodiments, the target gene is one or more of the target genes listed in Table 6 of US Pat. App. Pub. 2019/0255107, as being associated with the early stage of Th17 differentiation, maintenance and/or function, e.g., FAS, CCR5, IL6ST, IL17RA, IL2RA, MYD88, CXCR5, PVR, IL15RA, IL12RB1, or any combination thereof.

In some embodiments, the target gene is one or more of the target genes listed in Table 6 of US Pat. App. Pub. 2019/0255107 as being associated with the intermediate stage of Th17 differentiation, maintenance and/or function, e.g., IL7R, ITGA3, IL1R1, CCR5, CCR6, ACVR2A, IL6ST, IL17RA, CCR8, DDR1, PROCR, IL2RA, IL12RB2, MYD88, PTPRJ, TNFRSF13B, CXCR3, IL1RN, CXCR5, CCR4, IL4R, IL2RB, TNFRSF12A, CXCR4, KLRD1, IRAK1BP1, PVR, IL12RB1, IL18R1, TRAF3, or any combination thereof.

In some embodiments, the target gene is one or more of the target genes listed in Table 6 of US Pat. App. Pub. 2019/0255107 as being associated with the late stage of Th17 differentiation, maintenance and/or function, e.g., IL7R, ITGA3, IL1R1, FAS, CCR5, CCR6, ACVR2A, IL6ST, IL17RA, DDR1, PROCR, IL2RA, IL12RB2, MYD88, BMPR1A, PTPRJ, TNFRSF13B, CXCR3, IL1RN, CXCR5, CCR4, IL4R, IL2RB, TNFRSF12A, CXCR4, KLRD1, IRAK1BP1, PVR, IL15RA, TLR1, ACVR1B, IL12RB1, IL18R1, TRAF3, IFNGR1, PLAUR, IL21R, IL23R, or any combination thereof.

In some embodiments, the target gene is one or more of the target genes listed in Table 7 of US Pat. App. Pub. 2019/0255107 as being associated with the early stage of Th17 differentiation, maintenance and/or function, e.g., EIF2AK2, DUSP22, HK2, RIPK1, RNASEL, TEC, MAP3K8, SGK1, PRKCQ, DUSP16, BMP2K, PIM2, or any combination thereof.

In some embodiments, the target gene is one or more of the target genes listed in Table 7 of US Pat. App. Pub. 2019/0255107 as being associated with the intermediate stage of Th17 differentiation, maintenance and/or function, e.g., PSTPIP1, PTPN1, ACP5, TXK, RIPK3, PTPRF, NEK4, PPME1, PHACTR2, HK2, GMFG, DAPP1, TEC, GMFB, PIM1, NEK6, ACVR2A, FES, CDK6, ZAK, DUSP14, SGK1, JAK3, ULK2, PTPRJ, SPHK1, TNK2, PCTK1, MAP4K3, TGFBR1, HK1, DDR1, BMP2K, DUSP10, ALPK2, or any combination thereof.

In some embodiments, the target gene is one or more of the target genes listed in v Table 7 of US Pat. App. Pub. 2019/0255107 as being associated with the late stage of Th17 differentiation, maintenance and/or function, e.g., PTPLA, PSTPIP1, TK1, PTEN, BPGM, DCK, PTPRS, PTPN18, MKNK2, PTPN1, PTPRE, SH2D1A, PLK2, DUSP6, CDC25B, SLK, MAP3K5, BMPR1A, ACP5, TXK, RIPK3, PPP3CA, PTPRF, PACSIN1, NEK4, PIP4K2A, PPME1, SRPK2, DUSP2, PHACTR2, DCLK1, PPP2R5A, RIPK1, GK, RNASEL, GMFG, STK4, HINT3, DAPP1, TEC, GMFB, PTPN6, RIPK2, PIM1, NEK6, ACVR2A, AURKB, FES, ACVR1B, CDK6, ZAK, VRK2, MAP3K8, DUSP14, SGK1, PRKCQ, JAK3, ULK2, HIPK2, PTPRJ, INPP1, TNK2, PCTK1, DUSP1, NUDT4, TGFBR1, PTP4A1, HK1, DUSP16, ANP32A, DDR1, ITK, WNK1, NAGK, STK38, BMP2K, BUB1, AAK1, SIK1, DUSP10, PRKCA, PIM2, STK17B, TK2, STK39, ALPK2, MST4, PHLPP1, or any combination thereof.

In some embodiments, the target gene is one or more of the target genes listed in Table 8 of US Pat. App. Pub. 2019/0255107 as being associated with the early stage of Th17 differentiation, maintenance and/or function, e.g., HK2, CDKN1A, DUT, DUSP1, NADK, LIMK2, DUSP11, TAOK3, PRPS1, PPP2R4, MKNK2, SGK1, BPGM, TEC, MAPK6, PTP4A2, PRPF4B, ACP1, CCRN4L, or any combination thereof.

In some embodiments, the target gene is one or more of the target genes listed in Table 8 of US Pat. App. Pub. 2019/0255107 as being associated with the intermediate stage of Th17 differentiation, maintenance and/or function, e.g., HK2, ZAP70, NEK6, DUSP14, SH2D1A, ITK, DUT, PPP1R11, DUSP1, PMVK, TK1, TAOK3, GMFG, PRPS1, SGK1, TXK, WNK1, DUSP19, TEC, RPS6KA1, PKM2, PRPF4B, ADRBK1, CKB, ULK2, PLK1, PPP2R5A, PLK2, or any combination thereof.

In some embodiments, the target gene is one or more of the target genes listed in T Table 8 of US Pat. App. Pub. 2019/0255107 as being associated with the late stage of Th17 differentiation, maintenance and/or function, e.g., ZAP70, PFKP, NEK6, DUSP14, SH2D1A, INPP5B, ITK, PFKL, PGK1, CDKN1A, DUT, PPP1R11, DUSP1, PMVK, PTPN22, PSPH, TK1, PGAM1, LIMK2, CLK1, DUSP11, TAOK3, RIOK2, GMFG, UCKL1, PRPS1, PPP2R4, MKNK2, DGKA, SGK1, TXK, WNK1, DUSP19, CHP, BPGM, PIP5K1A, TEC, MAP2K1, MAPK6, RPS6KA1, PTP4A2, PKM2, PRPF4B, ADRBK1, CKB, ACP1, ULK2, CCRN4L, PRKCH, PLK1, PPP2R5A, PLK2, or any combination thereof.

In some embodiments, the target gene is one or more of the target genes listed in Table 9 of US Pat. App. Pub. 2019/0255107 as being associated with the early stage of Th17 differentiation, maintenance and/or function, e.g., CD200, CD40LG, CD24, CCND2, ADAM17, BSG, ITGAL, FAS, GPR65, SIGMAR1, CAP1, PLAUR, SRPRB, TRPV2, IL2RA, KDELR2, TNFRSF9, or any combination thereof.

In some embodiments, the target gene is one or more of the target genes listed in Table 9 of US Pat. App. Pub. 2019/0255107 as being associated with the intermediate stage of Th17 differentiation, maintenance and/or function, e.g., CTLA4, CD200, CD24, CD5L, CD9, IL2RB, CD53, CD74, CAST, CCR6, IL2RG, ITGAV, FAS, IL4R, PROCR, GPR65, TNFRSF18, RORA, IL1RN, RORC, CYSLTR1, PNRC2, LOC390243, ADAM10, TNFSF9, CD96, CD82, SLAMF7, CD27, PGRMC1, TRPV2, ADRBK1, TRAF6, IL2RA, THY1, IL12RB2, TNFRSF9, or any combination thereof.

In some embodiments, the target gene is one or more of the target genes listed in Table 9 of US Pat. App. Pub. 2019/0255107 as being associated with the late stage of Th17 differentiation, maintenance and/or function, e.g., CTLA4, TNFRSF4, CD44, PDCD1, CD200, CD247, CD24, CD5L, CCND2, CD9, IL2RB, CD53, CD74, ADAM17, BSG, CAST, CCR6, IL2RG, CD81, CD6, CD48, ITGAV, TFRC, ICAM2, ATP1B3, FAS, IL4R, CCR7, CD52, PROCR, GPR65, TNFRSF18, FCRL1, RORA, IL1RN, RORC, P2RX4, SSR2, PTPN22, SIGMAR1, CYSLTR1, LOC390243, ADAM10, TNFSF9, CD96, CAP1, CD82, SLAMF7, PLAUR, CD27, SIVA1, PGRMC1, SRPRB, TRPV2, NR1H2, ADRBK1, GABARAPL1, TRAF6, IL2RA, THY1, KDELR2, IL12RB2, TNFRSF9, SCARB1, IFNGR1, or any combination thereof.

In some embodiments, the target gene is one or more target genes that is a promoter of Th17 cell differentiation. In some embodiments, the target gene is GPR65. In some embodiments, the target gene is also a promoter of pathogenic Th17 cell differentiation and is selected from the group consisting of CD5L, DEC1, PLZP and TCF4.

In some embodiments, the target gene is one or more target genes that is a promoter of pathogenic Th17 cell differentiation. In some embodiments, the target gene is selected from the group consisting of CD5L, DEC1, PLZP and TCF4.

The desired gene or combination of target genes is selected, and after determining whether the selected target gene(s) is overexpressed or under-expressed during Th17 differentiation and/or Th17 maintenance, a suitable antagonist or agonist is used depending on the desired differentiation, maintenance and/or function outcome. For example, for target genes that are identified as positive regulators of Th17 differentiation, use of an antagonist that interacts with those target genes will shift differentiation away from the Th17 phenotype, while use of an agonist that interacts with those target genes will shift differentiation toward the Th17 phenotype. For target genes that are identified as negative regulators of Th17 differentiation, use of an antagonist that interacts with those target genes will shift differentiation toward from the Th17 phenotype, while use of an agonist that interacts with those target genes will shift differentiation away the Th17 phenotype. For example, for target genes that are identified as positive regulators of Th17 maintenance, use of an antagonist that interacts with those target genes will reduce the number of cells with the Th17 phenotype, while use of an agonist that interacts with those target genes will increase the number of cells with the Th17 phenotype. For target genes that are identified as negative regulators of Th17 differentiation, use of an antagonist that interacts with those target genes will increase the number of cells with the Th17 phenotype, while use of an agonist that interacts with those target genes will reduce the number of cells with the Th17 phenotype. Suitable T cell modulating agents include an antibody, a soluble polypeptide, a polypeptide agent, a peptide agent, a nucleic acid agent, a nucleic acid ligand, or a small molecule agent.

In some embodiments, the positive regulator of Th17 differentiation is a target gene selected from MINA, TRPS1, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3, and combinations thereof. In some embodiments, the positive regulator of Th17 differentiation is a target gene selected from MINA, PML, POU2AF1, PROCR, SMARCA4, ZEB1, EGR2, CCR6, FAS and combinations thereof.

In some embodiments, the negative regulator of Th17 differentiation is a target gene selected from SP4, ETS2, IKZF4, TSC22D3, IRF1 and combinations thereof. In some embodiments, the negative regulator of Th17 differentiation is a target gene selected from SP4, IKZF4, TSC22D3 and combinations thereof.

In some embodiments, the T cell modulating agent is a soluble Fas polypeptide or a polypeptide derived from FAS. In some embodiments, the T cell modulating agent is an agent that enhances or otherwise increases the expression, activity, and/or function of FAS in Th17 cells. As shown herein, expression of FAS in T cell populations induced or otherwise influenced differentiation toward Th17 cells. In some embodiments, these T cell modulating agents are useful in the treatment of an immune response, for example, an autoimmune response or an inflammatory response. In some embodiments, these T cell modulating agents are useful in the treatment of an infectious disease or other pathogen-based disorders. In some embodiments, the T cell modulating agent is an antibody, a soluble polypeptide, a polypeptide agonist, a peptide agonist, a nucleic acid agonist, a nucleic acid ligand, or a small molecule agonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of FAS. Inhibition of FAS expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, these T cell modulating agents are useful in the treatment of autoimmune diseases such as psoriasis, inflammatory bowel disease (IBD), ankylosing spondylitis, multiple sclerosis, Sjögren's syndrome, uveitis, and rheumatoid arthritis, asthma, systemic lupus erythematosus, transplant rejection including allograft rejection, and combinations thereof. In addition, enhancement of Th17 cells is also useful for clearing fungal infections and extracellular pathogens. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells that express additional cytokines. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of CCR5. Inhibition of CCR5 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an inhibitor or neutralizing agent. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of CCR6. Inhibition of CCR6 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of EGR1. Inhibition of EGR1 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of EGR2. Inhibition of EGR2 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

For example, in some embodiments, the invention provides T cell modulating agents and methods of using these T cell modulating agents to regulate, influence or otherwise impact the phenotype of a Th17 cell or population of cells, for example, by influencing a naïve T cell or population of cells to differentiate to a pathogenic or non-pathogenic Th17 cell or population of cells, by causing a pathogenic Th17 cell or population of cells to switch to a non-pathogenic Th17 cell or population of T cells (e.g., populations of naïve T cells, partially differentiated T cells, differentiated T cells and combinations thereof), or by causing a non-pathogenic Th17 cell or population of T cells (e.g., populations of naïve T cells, partially differentiated T cells, differentiated T cells and combinations thereof) to switch to a pathogenic Th17 cell or population of cells.

In some embodiments, the invention comprises a method of drug discovery for the treatment of a disease or condition involving an immune response involving T cell balance in a population of cells or tissue of a target gene comprising the steps of providing a compound or plurality of compounds to be screened for their efficacy in the treatment of said disease or condition, contacting said compound or plurality of compounds with said population of cells or tissue, detecting a first level of expression, activity and/or function of a target gene, comparing the detected level to a control of level of a target gene, and evaluating the difference between the detected level and the control level to determine the immune response elicited by said compound or plurality of compounds. For example, the method contemplates comparing tissue samples which can be inter alia infected tissue, inflamed tissue, healthy tissue, or combinations of tissue samples thereof.

In one embodiment of the invention, the reductase null animals of the present invention may advantageously be used to modulate T cell balance in a tissue or cell specific manner. Such animals may be used for the applications hereinbefore described, where the role of T cell balance in product/drug metabolism, detoxification, normal homeostasis or in disease etiology is to be studied. It is envisaged that this embodiment will also allow other effects, such as drug transporter-mediated effects, to be studied in those tissues or cells in the absence of metabolism, e.g., carbon metabolism. Accordingly, the animals of the present invention, in a further aspect of the invention may be used to modulate the functions and antibodies in any of the above cell types to generate a disease model or a model for product/drug discovery or a model to verify or assess functions of T cell balance.

In another embodiment, the method contemplates use of animal tissues and/or a population of cells derived therefrom of the present invention as an in vitro assay for the study of any one or more of the following events/parameters: (i) role of transporters in product uptake and efflux; (ii) identification of product metabolites produced by T cells; (iii) evaluate whether candidate products are T cells; or (iv) assess drug/drug interactions due to T cell balance.

The terms “pathogenic” or “non-pathogenic” as used herein are not to be construed as implying that one Th17 cell phenotype is more desirable than the other. As described herein, there are instances in which inhibiting the induction of pathogenic Th17 cells or modulating the Th17 phenotype towards the non-pathogenic Th17 phenotype is desirable. Likewise, there are instances where inhibiting the induction of non-pathogenic Th17 cells or modulating the Th17 phenotype towards the pathogenic Th17 phenotype is desirable.

As used herein, terms such as “pathogenic Th17 cell” and/or “pathogenic Th17 phenotype” and all grammatical variations thereof refer to Th17 cells that, when induced in the presence of TGF-β3, express an elevated level of one or more genes selected from Cxcl3, IL22, IL3, Ccl4, Gzmb, Lrmp, Ccl5, Casp1, Csf2, Ccl3, Tbx21, Icos, IL17r, Stat4, Lgals3 and Lag, as compared to the level of expression in a TGF-β3-induced Th17 cells. As used herein, terms such as “non-pathogenic Th17 cell” and/or “non-pathogenic Th17 phenotype” and all grammatical variations thereof refer to Th17 cells that, when induced in the presence of TGF-β3, express a decreased level of one or more genes selected from IL6st, IL1rn, Ikzf3, Maf, Ahr, IL9 and IL10, as compared to the level of expression in a TGF-β3-induced Th17 cells.

In some embodiments, the T cell modulating agent is an agent that enhances or otherwise increases the expression, activity and/or function of Protein C Receptor (PROCR, also called EPCR or CD201) in Th17 cells. As shown herein, expression of PROCR in Th17 cells reduced the pathogenicity of the Th17 cells, for example, by switching Th17 cells from a pathogenic to non-pathogenic signature. Thus, PROCR and/or these agonists of PROCR are useful in the treatment of a variety of indications, particularly in the treatment of aberrant immune response, for example in autoimmune diseases and/or inflammatory disorders. In some embodiments, the T cell modulating agent is an antibody, a soluble polypeptide, a polypeptide agonist, a peptide agonist, a nucleic acid agonist, a nucleic acid ligand, or a small molecule agonist.

In some embodiments, the T cell modulating agent is an agent that inhibits the expression, activity and/or function of the Protein C Receptor (PROCR, also called EPCR or CD201). Inhibition of PROCR expression, activity and/or function in Th17 cells switches non-pathogenic Th17 cells to pathogenic Th17 cells. Thus, these PROCR antagonists are useful in the treatment of a variety of indications, for example, infectious disease and/or other pathogen-based disorders. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cell modulating agent is a soluble Protein C Receptor (PROCR, also called EPCR or CD201) polypeptide or a polypeptide derived from PROCR.

In some embodiments, the invention provides a method of inhibiting Th17 differentiation, maintenance and/or function in a cell population and/or increasing expression, activity and/or function of one or more non-Th17-associated cytokines, one or more non-Th17 associated receptor molecules, or non-Th17-associated transcription regulators selected from FOXP3, interferon gamma (IFN-γ), GATA3, STAT4 and TBX21, comprising contacting a T cell with an agent that inhibits expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent inhibits expression, activity and/or function of at least one of MINA, PML, POU2AF1, PROCR, SMARCA4, ZEB1, EGR2, CCR6, FAS or combinations thereof. In some embodiments, the agent is an antibody, a soluble polypeptide, a polypeptide antagonist, a peptide antagonist, a nucleic acid antagonist, a nucleic acid ligand, or a small molecule antagonist. In some embodiments, the antibody is a monoclonal antibody. In some embodiments, the antibody is a chimeric, humanized or fully human monoclonal antibody. In some embodiments, the T cell is a naïve T cell, and wherein the agent is administered in an amount that is sufficient to modulate the phenotype of the T cell to become and/or produce a desired non-Th17 T cell phenotype, for example, a regulatory T cell (Treg) phenotype or another CD4+ T cell phenotype. In some embodiments, the T cell is a partially differentiated T cell, and wherein the agent is administered in an amount that is sufficient to modulate the phenotype of the partially differentiated T cell to become and/or produce a desired non-Th17 T cell phenotype, for example, a regulatory T cell (Treg) phenotype or another CD4+ T cell phenotype. In some embodiments, the T cell is a Th17 T cell, and wherein the agent is administered in an amount that is sufficient to modulate the phenotype of the Th17 T cell to become and/or produce a CD4+ T cell phenotype other than a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of inhibiting Th17 differentiation in a cell population and/or increasing expression, activity and/or function of one or more non-Th17-associated cytokines, one or more non-Th17-associated receptor molecules, or non-Th17-associated transcription factor selected from FOXP3, interferon gamma (IFN-γ), GATA3, STAT4 and TBX21, comprising contacting a T cell with an agent that enhances expression, activity and/or function of SP4, ETS2, IKZF4, TSC22D3, IRF1 or combinations thereof. In some embodiments, the agent enhances expression, activity and/or function of at least one of SP4, IKZF4, TSC22D3 or combinations thereof. In some embodiments, the agent is an antibody, a soluble polypeptide, a polypeptide agonist, a peptide agonist, a nucleic acid agonist, a nucleic acid ligand, or a small molecule agonist. In some embodiments, the antibody is a monoclonal antibody. In some embodiments, the T cell is a naïve T cell, and wherein the agent is administered in an amount that is sufficient to modulate the phenotype of the T cell to become and/or produce a desired non-Th17 T cell phenotype, for example, a regulatory T cell (Treg) phenotype or another CD4+ T cell phenotype. In some embodiments, the T cell is a partially differentiated T cell, and wherein the agent is administered in an amount that is sufficient to modulate the phenotype of the partially differentiated T cell to become and/or produce a desired non-Th17 T cell phenotype, for example, a regulatory T cell (Treg) phenotype or another CD4+ T cell phenotype. In some embodiments, the T cell is a Th17 T cell, and wherein the agent is administered in an amount that is sufficient to modulate the phenotype of the Th17 T cell to become and/or produce a CD4+ T cell phenotype other than a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of enhancing Th17 differentiation in a cell population increasing expression, activity and/or function of one or more Th17-associated cytokines, one or more Th17-associated receptor molecules, or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines, one or more Th17-associated receptor molecules, or one or more non-Th17-associated transcription regulators selected from FOXP3, interferon gamma (IFN-γ), GATA3, STAT4 and TBX21, comprising contacting a T cell with an agent that inhibits expression, activity and/or function of SP4, ETS2, IKZF4, TSC22D3, IRF1 or combinations thereof. In some embodiments, the agent inhibits expression, activity and/or function of at least one of SP4, IKZF4, TSC22D3 or combinations thereof. In some embodiments, the agent is an antibody, a soluble polypeptide, a polypeptide antagonist, a peptide antagonist, a nucleic acid antagonist, a nucleic acid ligand, or a small molecule antagonist. In some embodiments, the antibody is a monoclonal antibody. In some embodiments, the antibody is a chimeric, humanized or fully human monoclonal antibody. In some embodiments, the T cell is a naïve T cell, and wherein the agent is administered in an amount that is sufficient to modulate the phenotype of the T cell to become and/or produce a desired Th17 T cell phenotype. In some embodiments, the T cell is a partially differentiated T cell, and wherein the agent is administered in an amount that is sufficient to modulate the phenotype of the partially differentiated T cell to become and/or produce a desired Th17 T cell phenotype. In some embodiments, the T cell is a CD4+ T cell other than a Th17 T cell, and wherein the agent is administered in an amount that is sufficient to modulate the phenotype of the non-Th17 T cell to become and/or produce a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of enhancing Th17 differentiation in a cell population, increasing expression, activity and/or function of one or more Th17-associated cytokines, one or more Th17-associated receptor molecules, and/or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines, one or more Th17-associated receptor molecules, or one or more non-Th17-associated transcription regulators selected from FOXP3, interferon gamma (IFN-γ), GATA3, STAT4 and TBX21, comprising contacting a T cell with an agent that enhances expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent enhances expression, activity and/or function of at least one of MINA, PML, POU2AF1, PROCR, SMARCA4, ZEB1, EGR2, CCR6, FAS or combinations thereof. In some embodiments, the agent is an antibody, a soluble polypeptide, a polypeptide agonist, a peptide agonist, a nucleic acid agonist, a nucleic acid ligand, or a small molecule agonist. In some embodiments, the antibody is a monoclonal antibody. In some embodiments, the antibody is a chimeric, humanized or fully human monoclonal antibody. In some embodiments, the agent is administered in an amount sufficient to inhibit Foxp3, IFN-γ, GATA3, STAT4 and/or TBX21 expression, activity and/or function. In some embodiments, the T cell is a naïve T cell, and wherein the agent is administered in an amount that is sufficient to modulate the phenotype of the T cell to become and/or produce a desired Th17 T cell phenotype. In some embodiments, the T cell is a partially differentiated T cell, and wherein the agent is administered in an amount that is sufficient to modulate the phenotype of the partially differentiated T cell to become and/or produce a desired Th17 T cell phenotype. In some embodiments, the T cell is a CD4+ T cell other than a Th17 T cell, and wherein the agent is administered in an amount that is sufficient to modulate the phenotype of the non-Th17 T cell to become and/or produce a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of identifying genes or genetic elements associated with Th17 differentiation comprising: a) contacting a T cell with an inhibitor of Th17 differentiation or an agent that enhances Th17 differentiation; and b) identifying a gene or genetic element whose expression is modulated by step (a). In some embodiments, the method also comprises c) perturbing expression of the gene or genetic element identified in step b) in a T cell that has been in contact with an inhibitor of Th17 differentiation or an agent that enhances Th17 differentiation; and d) identifying a gene whose expression is modulated by step c). In some embodiments, the inhibitor of Th17 differentiation is an agent that inhibits the expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent inhibits expression, activity and/or function of at least one of MINA, PML, POU2AF1, PROCR, SMARCA4, ZEB1, EGR2, CCR6, FAS or combinations thereof. In some embodiments, the inhibitor of Th17 differentiation is an agent that enhances expression, activity and/or function of SP4, ETS2, IKZF4, TSC22D3, IRF1 or combinations thereof. In some embodiments, the agent enhances expression, activity and/or function of at least one of SP4, IKZF4 or TSC22D3. In some embodiments, the agent that enhances Th17 differentiation is an agent that inhibits expression, activity and/or function of SP4, ETS2, IKZF4, TSC22D3, IRF1 or combinations thereof. In some embodiments, wherein the agent that enhances Th17 differentiation is an agent that enhances expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent is an antibody, a soluble polypeptide, a polypeptide antagonist, a peptide antagonist, a nucleic acid antagonist, a nucleic acid ligand, or a small molecule antagonist.

In some embodiments, the invention provides a method of modulating induction of Th17 differentiation comprising contacting a T cell with an agent that modulates expression, activity and/or function of one or more target genes or one or more products of one or more target genes selected from IRF1, IRF8, IRF9, STAT2, STAT3, IRF7, STAT1, ZFP281, IFI35, REL, TBX21, FLI1, BATF, IRF4, one or more of the target genes listed in Table 5 of US Pat. App. Pub. 2019/0255107 as being associated with the early stage of Th17 differentiation, maintenance and/or function, e.g., AES, AHR, ARID5A, BATF, BCL11B, BCL3, CBFB, CBX4, CHD7, CITED2, CREB1, E2F4, EGR1, EGR2, ELL2, ETS1, ETS2, ETV6, EZH1, FLI1, FOXO1, GATA3, GATAD2B, HIF1A, ID2, IFI35, IKZF4, IRF1, IRF2, IRF3, IRF4, IRF7, IRF9, JMJD1C, JUN, LEF1, LRRFIP1, MAX, NCOA3, NFE2L2, NFIL3, NFKB1, NMI, NOTCH1, NR3C1, PHF21A, PML, PRDM1, REL, RELA, RUNX1, SAP18, SATB1, SMAD2, SMARCA4, SP100, SP4, STAT1, STAT2, STAT3, STAT4, STAT5B, STAT6, TFEB, TP53, TRIM24, and/or ZFP161, or any combination thereof.

In some embodiments, the invention provides a method of modulating onset of Th17 phenotype and amplification of Th17 T cells comprising contacting a T cell with an agent that modulates expression, activity and/or function of one or more target genes or one or more products of one or more target genes selected from IRF8, STAT2, STAT3, IRF7, JUN, STAT5B, ZPF2981, CHD7, TBX21, FLI1, SATB1, RUNX1, BATF, RORC, SP4, one or more of the target genes listed in Table 5 of US Pat. App. Pub. 2019/0255107 as being associated with the intermediate stage of Th17 differentiation, maintenance and/or function, e.g., AES, AHR, ARID3A, ARID5A, ARNTL, ASXL1, BATF, BCL11B, BCL3, BCL6, CBFB, CBX4, CDC5L, CEBPB, CHD7, CREB1, CREB3L2, CREM, E2F4, E2F8, EGR1, EGR2, ELK3, ELL2, ETS1, ETS2, ETV6, EZH1, FLI1, FOSL2, FOXJ2, FOXO1, FUS, HIF1A, HMGB2, ID1, ID2, IFI35, IKZF4, IRF3, IRF4, IRF7, IRF8, IRF9, JUN, JUNB, KAT2B, KLF10, KLF6, KLF9, LEF1, LRRFIP1, MAFF, MAX, MAZ, MINA, MTA3, MYC, MYST4, NCOA1, NCOA3, NFE2L2, NFIL3, NFKB1, NMI, NOTCH1, NR3C1, PHF21A, PML, POU2AF1, POU2F2, PRDM1, RARA, RBPJ, RELA, RORA, RUNX1, SAP18, SATB1, SKI, SKIL, SMAD2, SMAD7, SMARCA4, SMOX, SP1, SP4, SS18, STAT1, STAT2, STAT3, STAT5A, STAT5B, STAT6, SUZ12, TBX21, TFEB, TLE1, TP53, TRIM24, TRIM28, TRPS1, VAV1, ZEB1, ZEB2, ZFP161, ZFP62, ZNF238, ZNF281, and/or ZNF703, or any combination thereof.

In some embodiments, the invention provides a method of modulating stabilization of Th17 cells and/or modulating Th17-associated interleukin 23 (IL-23) signaling comprising contacting a T cell with an agent that modulates expression, activity and/or function of one or more target genes or one or more products of one or more target genes selected from STAT2, STAT3, JUN, STAT5B, CHD7, SATB1, RUNX1, BATF, RORC, SP4 IRF4, one or more of the target genes listed in Table 5 of US Pat. App. Pub. 2019/0255107 as being associated with the late stage of Th17 differentiation, maintenance and/or function, e.g., AES, AHR, ARID3A, ARID5A, ARNTL, ASXL1, ATF3, ATF4, BATF, BATF3, BCL11B, BCL3, BCL6, C210RF66, CBFB, CBX4, CDC5L, CDYL, CEBPB, CHD7, CHMP1B, CIC, CITED2, CREB1, CREB3L2, CREM, CSDA, DDIT3, E2F1, E2F4, E2F8, EGR1, EGR2, ELK3, ELL2, ETS1, ETS2, EZH1, FLI1, FOSL2, FOXJ2, FOXO1, FUS, GATA3, GATAD2B, HCLS1, HIF1A, ID1, ID2, IFI35, IKZF4, IRF3, IRF4, IRF7, IRF8, IRF9, JARID2, JMJD1C, JUN, JUNB, KAT2B, KLF10, KLF6, KLF7, KLF9, LASS4, LEF1, LRRFIP1, MAFF, MAX, MEN1, MINA, MTA3, MXI1, MYC, MYST4, NCOA1, NCOA3, NFE2L2, NFIL3, NFKB1, NMI, NOTCH1, NR3C1, PHF13, PHF21A, PML, POU2AF1, POU2F2, PRDM1, RARA, RBPJ, REL, RELA, RNF11, RORA, RORC, RUNX1, RUNX2, SAP18, SAP30, SATB1, SERTAD1, SIRT2, SKI, SKIL, SMAD2, SMAD4, SMAD7, SMARCA4, SMOX, SP1, SP100, SP4, SS18, STAT1, STAT3, STAT4, STAT5A, STAT5B, STAT6, SUZ12, TBX21, TFEB, TGIF1, TLE1, TP53, TRIM24, TRPS1, TSC22D3, UBE2B, VAV1, VAX2, XBP1, ZEB1, ZEB2, ZFP161, ZFP36L1, ZFP36L2, ZNF238, ZNF281, ZNF703, ZNRF1, and/or ZNRF2, or any combination thereof.

In some embodiments, the invention provides a method of modulating one or more of the target genes listed in Table 6 of US Pat. App. Pub. 2019/0255107 as being associated with the early stage of Th17 differentiation, maintenance and/or function, e.g., FAS, CCR5, IL6ST, IL17RA, IL2RA, MYD88, CXCR5, PVR, IL15RA, IL12RB1, or any combination thereof.

In some embodiments, the invention provides a method of modulating one or more of the target genes listed in Table 6 of US Pat. App. Pub. 2019/0255107 as being associated with the intermediate stage of Th17 differentiation, maintenance and/or function, e.g., IL7R, ITGA3, IL1R1, CCR5, CCR6, ACVR2A, IL6ST, IL17RA, CCR8, DDR1, PROCR, IL2RA, IL12RB2, MYD88, PTPRJ, TNFRSF13B, CXCR3, IL1RN, CXCR5, CCR4, IL4R, IL2RB, TNFRSF12A, CXCR4, KLRD1, IRAK1BP1, PVR, IL12RB1, IL18R1, TRAF3, or any combination thereof.

In some embodiments, the invention provides a method of modulating one or more of the target genes listed in Table 6 of US Pat. App. Pub. 2019/0255107 as being associated with the late stage of Th17 differentiation, maintenance and/or function, e.g., IL7R, ITGA3, IL1R1, FAS, CCR5, CCR6, ACVR2A, IL6ST, IL17RA, DDR1, PROCR, IL2RA, IL12RB2, MYD88, BMPR1A, PTPRJ, TNFRSF13B, CXCR3, IL1RN, CXCR5, CCR4, IL4R, IL2RB, TNFRSF12A, CXCR4, KLRD1, IRAK1BP1, PVR, IL15RA, TLR1, ACVR1B, IL12RB1, IL18R1, TRAF3, IFNGR1, PLAUR, IL21R, IL23R, or any combination thereof.

In some embodiments, the invention provides a method of modulating one or more of the target genes listed in Table 7 of US Pat. App. Pub. 2019/0255107 as being associated with the early stage of Th17 differentiation, maintenance and/or function, e.g., EIF2AK2, DUSP22, HK2, RIPK1, RNASEL, TEC, MAP3K8, SGK1, PRKCQ, DUSP16, BMP2K, PIM2, or any combination thereof.

In some embodiments, the invention provides a method of modulating one or more of the target genes listed in Table 7 of US Pat. App. Pub. 2019/0255107 as being associated with the intermediate stage of Th17 differentiation, maintenance and/or function, e.g., PSTPIP1, PTPN1, ACP5, TXK, RIPK3, PTPRF, NEK4, PPME1, PHACTR2, HK2, GMFG, DAPP1, TEC, GMFB, PIM1, NEK6, ACVR2A, FES, CDK6, ZAK, DUSP14, SGK1, JAK3, ULK2, PTPRJ, SPHK1, TNK2, PCTK1, MAP4K3, TGFBR1, HK1, DDR1, BMP2K, DUSP10, ALPK2, or any combination thereof.

In some embodiments, the invention provides a method of modulating one or more of the target genes listed in Table 7 of US Pat. App. Pub. 2019/0255107 as being associated with the late stage of Th17 differentiation, maintenance and/or function, e.g., PTPLA, PSTPIP1, TK1, PTEN, BPGM, DCK, PTPRS, PTPN18, MKNK2, PTPN1, PTPRE, SH2D1A, PLK2, DUSP6, CDC25B, SLK, MAP3K5, BMPR1A, ACP5, TXK, RIPK3, PPP3CA, PTPRF, PACSIN1, NEK4, PIP4K2A, PPME1, SRPK2, DUSP2, PHACTR2, DCLK1, PPP2R5A, RIPK1, GK, RNASEL, GMFG, STK4, HINT3, DAPP1, TEC, GMFB, PTPN6, RIPK2, PIM1, NEK6, ACVR2A, AURKB, FES, ACVR1B, CDK6, ZAK, VRK2, MAP3K8, DUSP14, SGK1, PRKCQ, JAK3, ULK2, HIPK2, PTPRJ, INPP1, TNK2, PCTK1, DUSP1, NUDT4, TGFBR1, PTP4A1, HK1, DUSP16, ANP32A, DDR1, ITK, WNK1, NAGK, STK38, BMP2K, BUB1, AAK1, SIK1, DUSP10, PRKCA, PIM2, STK17B, TK2, STK39, ALPK2, MST4, PHLPP1, or any combination thereof.

In some embodiments, the invention provides a method of modulating is one or more of the target genes listed in Table 8 of US Pat. App. Pub. 2019/0255107 as being associated with the early stage of Th17 differentiation, maintenance and/or function, e.g., HK2, CDKN1A, DUT, DUSP1, NADK, LIMK2, DUSP11, TAOK3, PRPS1, PPP2R4, MKNK2, SGK1, BPGM, TEC, MAPK6, PTP4A2, PRPF4B, ACP1, CCRN4L, or any combination thereof.

In some embodiments, the invention provides a method of modulating one or more of the target genes listed in Table 8 of US Pat. App. Pub. 2019/0255107 as being associated with the intermediate stage of Th17 differentiation, maintenance and/or function, e.g., HK2, ZAP70, NEK6, DUSP14, SH2D1A, ITK, DUT, PPP1R11, DUSP1, PMVK, TK1, TAOK3, GMFG, PRPS1, SGK1, TXK, WNK1, DUSP19, TEC, RPS6KA1, PKM2, PRPF4B, ADRBK1, CKB, ULK2, PLK1, PPP2R5A, PLK2, or any combination thereof.

In some embodiments, the invention provides a method of modulating one or more of the target genes listed in Table 8 of US Pat. App. Pub. 2019/0255107 as being associated with the late stage of Th17 differentiation, maintenance and/or function, e.g., ZAP70, PFKP, NEK6, DUSP14, SH2D1A, INPP5B, ITK, PFKL, PGK1, CDKN1A, DUT, PPP1R11, DUSP1, PMVK, PTPN22, PSPH, TK1, PGAM1, LIMK2, CLK1, DUSP11, TAOK3, RIOK2, GMFG, UCKL1, PRPS1, PPP2R4, MKNK2, DGKA, SGK1, TXK, WNK1, DUSP19, CHP, BPGM, PIP5K1A, TEC, MAP2K1, MAPK6, RPS6KA1, PTP4A2, PKM2, PRPF4B, ADRBK1, CKB, ACP1, ULK2, CCRN4L, PRKCH, PLK1, PPP2R5A, PLK2, or any combination thereof.

In some embodiments, the invention provides a method of modulating one or more of the target genes listed in Table 9 of US Pat. App. Pub. 2019/0255107 as being associated with the early stage of Th17 differentiation, maintenance and/or function, e.g., CD200, CD40LG, CD24, CCND2, ADAM17, BSG, ITGAL, FAS, GPR65, SIGMAR1, CAP1, PLAUR, SRPRB, TRPV2, IL2RA, KDELR2, TNFRSF9, or any combination thereof.

In some embodiments, the invention provides a method of modulating one or more of the target genes listed in Table 9 of US Pat. App. Pub. 2019/0255107 as being associated with the intermediate stage of Th17 differentiation, maintenance and/or function, e.g., CTLA4, CD200, CD24, CD5L, CD9, IL2RB, CD53, CD74, CAST, CCR6, IL2RG, ITGAV, FAS, IL4R, PROCR, GPR65, TNFRSF18, RORA, IL1RN, RORC, CYSLTR1, PNRC2, LOC390243, ADAM10, TNFSF9, CD96, CD82, SLAMF7, CD27, PGRMC1, TRPV2, ADRBK1, TRAF6, IL2RA, THY1, IL12RB2, TNFRSF9, or any combination thereof.

In some embodiments, the invention provides a method of modulating one or more of the target genes listed in Table 9 of US Pat. App. Pub. 2019/0255107 as being associated with the late stage of Th17 differentiation, maintenance and/or function, e.g., CTLA4, TNFRSF4, CD44, PDCD1, CD200, CD247, CD24, CD5L, CCND2, CD9, IL2RB, CD53, CD74, ADAM17, BSG, CAST, CCR6, IL2RG, CD81, CD6, CD48, ITGAV, TFRC, ICAM2, ATP1B3, FAS, IL4R, CCR7, CD52, PROCR, GPR65, TNFRSF18, FCRL1, RORA, IL1RN, RORC, P2RX4, SSR2, PTPN22, SIGMAR1, CYSLTR1, LOC390243, ADAM10, TNFSF9, CD96, CAP1, CD82, SLAMF7, PLAUR, CD27, SIVA1, PGRMC1, SRPRB, TRPV2, NR1H2, ADRBK1, GABARAPL1, TRAF6, IL2RA, THY1, KDELR2, IL12RB2, TNFRSF9, SCARB1, IFNGR1, or any combination thereof.

In some embodiments, the invention provides a method of inhibiting tumor growth in a subject in need thereof by administering to the subject a therapeutically effective amount of an inhibitor of Protein C Receptor (PROCR). In some embodiments, the inhibitor of PROCR is an antibody, a soluble polypeptide, a polypeptide agent, a peptide agent, a nucleic acid agent, a nucleic acid ligand, or a small molecule agent. In some embodiments, the inhibitor of PROCR is one or more agents selected from the group consisting of lipopolysaccharide; cisplatin; fibrinogen; 1, 10-phenanthroline; 5-N-ethylcarboxamido adenosine; cystathionine; hirudin; phospholipid; Drotrecogin alfa; VEGF; Phosphatidylethanolamine; serine; gamma-carboxyglutamic acid; calcium; warfarin; endotoxin; curcumin; lipid; and nitric oxide.

In some embodiments, the invention provides a method of diagnosing an immune response in a subject, comprising detecting a level of expression, activity and/or function of one or more signature genes or one or more products of one or more signature genes selected from those listed in Table 1 of US Pat. App. Pub. 2019/0255107 or Table 1 herein and comparing the detected level to a control of level of signature gene or gene product expression, activity and/or function, wherein a difference between the detected level and the control level indicates that the presence of an immune response in the subject. In some embodiments, the immune response is an autoimmune response. In some embodiments, the immune response is an inflammatory response, including inflammatory response(s) associated with an autoimmune response and/or inflammatory response(s) associated with an infectious disease or other pathogen-based disorder.

In some embodiments, the invention provides a method of monitoring an immune response in a subject, comprising detecting a level of expression, activity and/or function of one or more signature genes or one or more products of one or more signature genes, e.g., one or more signature genes selected from those listed in Table 1 of US Pat. App. Pub. 2019/0255107 or Table 1 herein at a first time point, detecting a level of expression, activity and/or function of one or more signature genes or one or more products of one or more signature genes, e.g., one or more signature genes selected from those listed in Table 1 of US Pat. App. Pub. 2019/0255107 or Table 1 herein at a second time point, and comparing the first detected level of expression, activity and/or function with the second detected level of expression, activity and/or function, wherein a change between the first and second detected levels indicates a change in the immune response in the subject. In some embodiments, the immune response is an autoimmune response. In some embodiments, the immune response is an inflammatory response.

In some embodiments, the invention provides a method of monitoring an immune response in a subject, comprising isolating a population of T cells from the subject at a first time point, determining a first ratio of T cell subtypes within the T cell population at a first time point, isolating a population of T cells from the subject at a second time point, determining a second ratio of T cell subtypes within the T cell population at a second time point, and comparing the first and second ratio of T cell subtypes, wherein a change in the first and second detected ratios indicates a change in the immune response in the subject. In some embodiments, the immune response is an autoimmune response. In some embodiments, the immune response is an inflammatory response.

In some embodiments, the invention provides a method of activating therapeutic immunity by exploiting the blockade of immune checkpoints. The progression of a productive immune response requires that a number of immunological checkpoints be passed. Immunity response is regulated by the counterbalancing of stimulatory and inhibitory signal. The immunoglobulin superfamily occupies a central importance in this coordination of immune responses, and the CD28/cytotoxic T-lymphocyte antigen-4 (CTLA-4):B7.1/B7.2 receptor/ligand grouping represents the archetypal example of these immune regulators (see e.g., Korman A J, Peggs K S, Allison J P, “Checkpoint blockade in cancer immunotherapy.” Adv Immunol. 2006; 90:297-339). In part the role of these checkpoints is to guard against the possibility of unwanted and harmful self-directed activities. While this is a necessary function aiding in the prevention of autoimmunity, it may act as a barrier to successful immunotherapies aimed at targeting malignant self-cells that largely display the same array of surface molecules as the cells from which they derive. The expression of immune-checkpoint proteins can be dysregulated in a disease or disorder and can be an important immune resistance mechanism. Therapies aimed at overcoming these mechanisms of peripheral tolerance, in particular by blocking the inhibitory checkpoints, offer the potential to generate therapeutic activity, either as monotherapies or in synergism with other therapies.

Thus, the present invention relates to a method of engineering T-cells, especially for immunotherapy, comprising modulating T cell balance to inactivate or otherwise inhibit at least one gene or gene product involved in the immune check-point.

Suitable T cell modulating agent(s) for use in any of the compositions and methods provided herein include an antibody, a soluble polypeptide, a polypeptide agent, a peptide agent, a nucleic acid agent, a nucleic acid ligand, or a small molecule agent. By way of non-limiting example, suitable T cell modulating agents or agents for use in combination with one or more T cell modulating agents are shown in Table 10 of US Pat. App. Pub. 2019/0255107 of the specification.

One skilled in the art will appreciate that the T cell modulating agents have a variety of uses. For example, the T cell modulating agents are used as therapeutic agents as described herein. The T cell modulating agents can be used as reagents in screening assays, diagnostic kits or as diagnostic tools, or these T cell modulating agents can be used in competition assays to generate therapeutic reagents.

In one embodiment, the invention relates to a method of diagnosing, prognosing and/or staging an immune response involving T cell balance, which may comprise detecting a first level of expression, activity and/or function of one or more signature genes or one or more products of one or more signature genes selected from the genes of Table 1 of US Pat. App. Pub. 2019/0255107 or Table 1 herein and comparing the detected level to a control of level of signature gene or gene product expression, activity and/or function, wherein a difference in the detected level and the control level indicates the presence of an immune response in the subject.

In another embodiment, the invention relates to a method of monitoring an immune response in a subject comprising detecting a level of expression, activity and/or function of one or more signature genes or one or more products of one or more signature genes of Table 1 of US Pat. App. Pub. 2019/0255107 or Table 1 herein at a first time point, detecting a level of expression, activity and/or function of one or more signature genes or one or more products of one or more signature genes of Table 1 of US Pat. App. Pub. 2019/0255107 or Table 1 herein at a second time point, and comparing the first detected level of expression, activity and/or function with the second detected level of expression, activity and/or function, wherein a change in the first and second detected levels indicates a change in the immune response in the subject.

In yet another embodiment, the invention relates to a method of identifying a patient population at risk or suffering from an immune response which may comprise detecting a level of expression, activity and/or function of one or more signature genes or one or more products of one or more signature genes of Table 1 of US Pat. App. Pub. 2019/0255107 or Table 1 herein in the patient population and comparing the level of expression, activity and/or function of one or more signature genes or one or more products of one or more signature genes of Table 1 of US Pat. App. Pub. 2019/0255107 or Table 1 herein in a patient population not at risk or suffering from an immune response, wherein a difference in the level of expression, activity and/or function of one or more signature genes or one or more products of one or more signature genes of Table 1 of US Pat. App. Pub. 2019/0255107 or Table 1 herein in the patient populations identifies the patient population as at risk or suffering from an immune response.

In still another embodiment, the invention relates to a method for monitoring subjects undergoing a treatment or therapy for an aberrant immune response to determine whether the patient is responsive to the treatment or therapy which may comprise detecting a level of expression, activity and/or function of one or more signature genes or one or more products of one or more signature genes of Table 1 of US Pat. App. Pub. 2019/0255107 or Table 1 herein in the absence of the treatment or therapy and comparing the level of expression, activity and/or function of one or more signature genes or one or more products of one or more signature genes of Table 1 of US Pat. App. Pub. 2019/0255107 or Table 1 herein in the presence of the treatment or therapy, wherein a difference in the level of expression, activity and/or function of one or more signature genes or one or more products of one or more signature genes of Table 1 of US Pat. App. Pub. 2019/0255107 or Table 1 herein in the presence of the treatment or therapy indicates whether the patient is responsive to the treatment or therapy.

The invention may also involve a method of modulating T cell balance, the method which may comprise contacting a T cell or a population of T cells with a T cell modulating agent in an amount sufficient to modify differentiation, maintenance and/or function of the T cell or population of T cells by altering balance between Th17 cells, regulatory T cells (Tregs) and other T cell subsets as compared to differentiation, maintenance and/or function of the T cell or population of T cells in the absence of the T cell modulating agent.

The immune response may be an autoimmune response or an inflammatory response. The inflammatory response may be associated with an autoimmune response, an infectious disease and/or a pathogen-based disorder.

The signature genes may be Th17-associated genes.

The treatment or therapy may be an antagonist for GPR65 in an amount sufficient to induce differentiation toward regulatory T cells (Tregs), Th1 cells, or a combination of Tregs and Th1 cells. The treatment or therapy may be an agonist that enhances or increases the expression of GPR65 in an amount sufficient to induce T cell differentiation toward Th17 cells. The treatment or therapy may be specific for a target gene selected from the group consisting of DEC1, PZLP, TCF4 and CD5L. The treatment or therapy may be an antagonist of a target gene selected from the group consisting of DEC1, PZLP, TCF4 and CD5L in an amount sufficient to switch Th17 cells from a pathogenic to non-pathogenic signature. The treatment or therapy may be an agonist that enhances or increases the expression of a target gene selected from the group consisting of DEC1, PZLP, TCF4 and CD5L in an amount sufficient to switch Th17 cells from a non-pathogenic to a pathogenic signature.

The T cell modulating agent may be an antibody, a soluble polypeptide, a polypeptide agent, a peptide agent, a nucleic acid agent, a nucleic acid ligand, or a small molecule agent.

The T cells may be naïve T cells, partially differentiated T cells, differentiated T cells, a combination of naïve T cells and partially differentiated T cells, a combination of naïve T cells and differentiated T cells, a combination of partially differentiated T cells and differentiated T cells, or a combination of naïve T cells, partially differentiated T cells and differentiated T cells.

The invention also involves a method of enhancing Th17 differentiation in a cell population, increasing expression, activity and/or function of one or more Th17-associated cytokines or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines or non-Th17-associated transcription regulators selected from FOXP3, interferon gamma (IFN-γ), GATA3, STAT4 and TBX21, comprising contacting a T cell with an agent that enhances expression, activity and/or function of CD5L, DEC1, PLZP, TCF4 or combinations thereof. The agent may enhance expression, activity and/or function of at least one of CD5L, DEC1, PLZP, or TCF4. The agent may be an antibody, a soluble polypeptide, a polypeptide agonist, a peptide agonist, a nucleic acid agonist, a nucleic acid ligand, or a small molecule agonist. The antibody may be a monoclonal antibody or a chimeric, humanized or fully human monoclonal antibody.

The present invention also involves the use of an antagonist for GPR65 in an amount sufficient to induce differentiation toward regulatory T cells (Tregs), Th1 cells, or a combination of Tregs and Th1 cells, use of an agonist that enhances or increases the expression of GPR65 in an amount sufficient to induce T cell differentiation toward Th17 cells, use of an antagonist of a target gene selected from the group consisting of DEC1, PZLP, TCF4 and CD5L in an amount sufficient to switch Th17 cells from a pathogenic to non-pathogenic signature, use of an agonist that enhances or increases the expression of a target gene selected from the group consisting of DEC1, PZLP, TCF4 and CD5L in an amount sufficient to switch Th17 cells from a non-pathogenic to a pathogenic signature and use of T cell modulating agent for treating an aberrant immune response in a patient.

Methods of Diagnosing, Prognosing and/or Staging an Immune Response Involving T Cell Balance

The invention provides a method of diagnosing, prognosing and/or staging an immune response involving T cell balance, comprising detecting a first level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l or one or more products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l and comparing the detected level to a control of level of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l or gene product expression, activity and/or function, wherein a difference in the detected level and the control level indicates that the presence of an immune response in the subject.

The invention also provides a method of monitoring an immune response in a subject comprising detecting a level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l at a first time point, detecting a level of expression, activity and/or function of one or more signature genes or one or more products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l at a second time point, and comparing the first detected level of expression, activity and/or function with the second detected level of expression, activity and/or function, wherein a change in the first and second detected levels indicates a change in the immune response in the subject.

The invention also provides a method of identifying a patient population at risk or suffering from an immune response comprising detecting a level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l or one or more products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in the patient population and comparing the level of expression, activity and/or function of one or more signature genes or one or more products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in a patient population not at risk or suffering from an immune response, wherein a difference in the level of expression, activity and/or function of one or more of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l or one or more products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in the patient populations identifies the patient population as at risk or suffering from an immune response.

The invention also provides a method for monitoring subjects undergoing a treatment or therapy specific for a target gene selected from the group consisting of candidates Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l for an aberrant immune response to determine whether the patient is responsive to the treatment or therapy comprising detecting a level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in the absence of the treatment or therapy and comparing the level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in the presence of the treatment or therapy, wherein a difference in the level of expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l or products of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in the presence of the treatment or therapy indicates whether the patient is responsive to the treatment or therapy.

In these methods the immune response is an autoimmune response or an inflammatory response; or the inflammatory response is associated with an autoimmune response, an infectious disease and/or a pathogen-based disorder; or the signature genes are Th17-associated genes; or the treatment or therapy is an antagonist as to expression of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce differentiation toward regulatory T cells (Tregs), Th1 cells, or a combination of Tregs and Th1 cells; or the treatment or therapy is an agonist that enhances or increases the expression of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce T cell differentiation toward Th17 cells; or the treatment or therapy is an antagonist of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a pathogenic to non-pathogenic signature; or the treatment or therapy is an agonist that enhances or increases the expression of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a non-pathogenic to a pathogenic signature; or the T cell modulating agent is an antibody, a soluble polypeptide, a polypeptide agent, a peptide agent, a nucleic acid agent, a nucleic acid ligand, or a small molecule agent.

The invention also provides a method of modulating T cell balance, the method comprising contacting a T cell or a population of T cells with a T cell modulating agent in an amount sufficient to modify differentiation, maintenance and/or function of the T cell or population of T cells by altering balance between Th17 cells, regulatory T cells (Tregs) and other T cell subsets as compared to differentiation, maintenance and/or function of the T cell or population of T cells in the absence of the T cell modulating agent; wherein the T cell modulating agent is an antagonist for or of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce differentiation toward regulatory T cells (Tregs), Th1 cells, or a combination of Tregs and Th1 cells, or wherein the T cell modulating agent is an agonist that enhances or increases the expression of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce T cell differentiation toward Th17 cells, or wherein the T cell modulating agent is specific for a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l, or wherein the T cell modulating agent is an antagonist of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a pathogenic to non-pathogenic signature, or wherein the T cell modulating agent is an agonist that enhances or increases the expression of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a non-pathogenic to a pathogenic signature. In these methods the T cell modulating agent is an antibody, a soluble polypeptide, a polypeptide agent, a peptide agent, a nucleic acid agent, a nucleic acid ligand, or a small molecule agent; or the T cells are naïve T cells, partially differentiated T cells, differentiated T cells, a combination of naïve T cells and partially differentiated T cells, a combination of naïve T cells and differentiated T cells, a combination of partially differentiated T cells and differentiated T cells, or a combination of naïve T cells, partially differentiated T cells and differentiated T cells.

The invention also provides a method of enhancing Th17 differentiation in a cell population, increasing expression, activity and/or function of one or more Th17-associated cytokines or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines or non-Th17-associated transcription regulators selected from FOXP3, interferon gamma (IFN-γ), GATA3, STAT4 and TBX21, comprising contacting a T cell with an agent that enhances expression, activity and/or function of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l.

In methods herein the agent enhances expression, activity and/or function of at least Toso. The agent can be an antibody, a soluble polypeptide, a polypeptide agonist, a peptide agonist, a nucleic acid agonist, a nucleic acid ligand, or a small molecule agonist; advantageously an antibody, such as a monoclonal antibody; or an antibody that is a chimeric, humanized or fully human monoclonal antibody.

The invention comprehends use of an antagonist for or of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce differentiation toward regulatory T cells (Tregs), Th1 cells, or a combination of Tregs and Th1 cells for treating or Drug Discovery of or formulating or preparing a treatment for an aberrant immune response in a patient.

The invention comprehends use of an agonist that enhances or increases the expression of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to induce T cell differentiation toward Th17 cells for treating or Drug Discovery of or formulating or preparing a treatment for an aberrant immune response in a patient.

The invention comprehends use of an antagonist of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a pathogenic to non-pathogenic signature for treating or Drug Discovery of or formulating or preparing a treatment for an aberrant immune response in a patient.

The invention comprehends use of an agonist that enhances or increases the expression of a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l in an amount sufficient to switch Th17 cells from a non-pathogenic to a pathogenic signature for treating or Drug Discovery of or formulating or preparing a treatment for an aberrant immune response in a patient.

The invention comprehends a treatment method or Drug Discovery method or method of formulating or preparing a treatment comprising any one of the methods or uses herein discussed.

The invention comprehends a method of drug discovery for the treatment of a disease or condition involving an immune response involving T cell balance in a population of cells or tissue which express a target gene selected from the group consisting of Toso, advantageously Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l comprising the steps of (a) providing a compound or plurality of compounds to be screened for their efficacy in the treatment of said disease or condition; (b) contacting said compound or plurality of compounds with said population of cells or tissue; (c) detecting a first level of expression, activity and/or function of a target gene selected from the group consisting of Toso, Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination of Gpr65, Plzp or Cd5l in any combination thereof Gpr65, Plzp, Toso or Cd5l or one or more products of a target gene selected from the group consisting of Toso, Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination thereof Gpr65, Plzp or Cd5l in any combination of Gpr65, Plzp, Toso or Cd5l; (d) comparing the detected level to a control of level of a target gene selected from the group consisting of Toso, Ctla2b, Gatm, Bdh2, Bcat1, Zfp36, Acsl4, Acat3, Adi1, Dot1l, Mett10d, Sirt6, Slc25a13, Chd2, Ino80c, Med21, Pdss1, Galk1, Gnpda2 or Mtpap or any one of the foregoing or any combination thereof with one or more of Gpr65, Plzp or Cd5l or any combination of Gpr65, Plzp or Cd5l in any combination thereof Gpr65, Plzp, Toso or Cd5l or gene product expression, activity and/or function; (e) evaluating the difference between the detected level and the control level to determine the immune response elicited by said compound or plurality of compounds.

The invention provides compositions and methods for modulating T cell balance. As used herein, the term “modulating” includes up-regulation of, or otherwise increasing, the expression of one or more genes, down-regulation of, or otherwise decreasing, the expression of one or more genes, inhibiting or otherwise decreasing the expression, activity and/or function of one or more gene products, and/or enhancing or otherwise increasing the expression, activity and/or function of one or more gene products.

As used herein, the term “modulating T cell balance” includes the modulation of any of a variety of T cell-related functions and/or activities, including by way of non-limiting example, controlling or otherwise influencing the networks that regulate T cell differentiation; controlling or otherwise influencing the networks that regulate T cell maintenance, for example, over the lifespan of a T cell; controlling or otherwise influencing the networks that regulate T cell function; controlling or otherwise influencing the networks that regulate helper T cell (Th cell) differentiation; controlling or otherwise influencing the networks that regulate Th cell maintenance, for example, over the lifespan of a Th cell; controlling or otherwise influencing the networks that regulate Th cell function; controlling or otherwise influencing the networks that regulate Th17 cell differentiation; controlling or otherwise influencing the networks that regulate Th17 cell maintenance, for example, over the lifespan of a Th17 cell; controlling or otherwise influencing the networks that regulate Th17 cell function; controlling or otherwise influencing the networks that regulate regulatory T cell (Treg) differentiation; controlling or otherwise influencing the networks that regulate Treg cell maintenance, for example, over the lifespan of a Treg cell; controlling or otherwise influencing the networks that regulate Treg cell function; controlling or otherwise influencing the networks that regulate other CD4+ T cell differentiation; controlling or otherwise influencing the networks that regulate other CD4+ T cell maintenance; controlling or otherwise influencing the networks that regulate other CD4+ T cell function; manipulating or otherwise influencing the ratio of T cells such as, for example, manipulating or otherwise influencing the ratio of Th17 cells to other T cell types such as Tregs or other CD4+ T cells; manipulating or otherwise influencing the ratio of different types of Th17 cells such as, for example, pathogenic Th17 cells and non-pathogenic Th17 cells; manipulating or otherwise influencing at least one function or biological activity of a T cell; manipulating or otherwise influencing at least one function or biological activity of Th cell; manipulating or otherwise influencing at least one function or biological activity of a Treg cell; manipulating or otherwise influencing at least one function or biological activity of a Th17 cell; and/or manipulating or otherwise influencing at least one function or biological activity of another CD4⁺ T cell.

The invention provides T cell modulating agents that modulate T cell balance. For example, in some embodiments, the invention provides T cell modulating agents and methods of using these T cell modulating agents to regulate, influence or otherwise impact the level(s) of and/or balance between T cell types, e.g., between Th17 and other T cell types, for example, regulatory T cells (Tregs), and/or Th17 activity and inflammatory potential. As used herein, terms such as “Th17 cell” and/or “Th17 phenotype” and all grammatical variations thereof refer to a differentiated T helper cell that expresses one or more cytokines selected from the group consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

For example, in some embodiments, the invention provides T cell modulating agents and methods of using these T cell modulating agents to regulate, influence or otherwise impact the level of and/or balance between Th17 phenotypes, and/or Th17 activity and inflammatory potential. Suitable T cell modulating agents include an antibody, a soluble polypeptide, a polypeptide agent, a peptide agent, a nucleic acid agent, a nucleic acid ligand, or a small molecule agent.

For example, in some embodiments, the invention provides T cell modulating agents and methods of using these T cell modulating agents to regulate, influence or otherwise impact the level of and/or balance between Th17 cell types, e.g., between pathogenic and nonpathogenic Th17 cells. For example, in some embodiments, the invention provides T cell modulating agents and methods of using these T cell modulating agents to regulate, influence or otherwise impact the level of and/or balance between pathogenic and non-pathogenic Th17 activity.

For example, in some embodiments, the invention provides T cell modulating agents and methods of using these T cell modulating agents to influence or otherwise impact the differentiation of a population of T cells, for example toward Th17 cells, with or without a specific pathogenic distinction, or away from Th17 cells, with or without a specific pathogenic distinction.

For example, in some embodiments, the invention provides T cell modulating agents and methods of using these T cell modulating agents to influence or otherwise impact the differentiation of a population of T cells, for example toward a non-Th17 T cell subset or away from a non-Th17 cell subset. For example, in some embodiments, the invention provides T cell modulating agents and methods of using these T cell modulating agents to induce T-cell plasticity, i.e., converting Th17 cells into a different subtype, or into a new state.

For example, in some embodiments, the invention provides T cell modulating agents and methods of using these T cell modulating agents to induce T cell plasticity, e.g., converting Th17 cells into a different subtype, or into a new state.

For example, in some embodiments, the invention provides T cell modulating agents and methods of using these T cell modulating agents to achieve any combination of the above.

In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

The T cell modulating agents are used to modulate the expression of one or more target genes or one or more products of one or more target genes that have been identified as genes responsive to Th17-related perturbations. These target genes are identified, for example, by contacting a T cell, e.g., naïve T cells, partially differentiated T cells, differentiated T cells and/or combinations thereof, with a T cell modulating agent and monitoring the effect, if any, on the expression of one or more signature genes or one or more products of one or more signature genes. In some embodiments, the one or more signature genes are selected from those listed in Table 1 or Table 2 of WO/2014/134351, incorporated herein by reference; alone or with those of other herein disclosed methods.

In some embodiments, the target gene is one or more Th17-associated cytokine(s) or receptor molecule(s) selected from those listed in Table 3 of WO/2014/134351, incorporated herein by reference; alone or with those of other herein disclosed methods. In some embodiments, the target gene is one or more Th17-associated transcription regulator(s) selected from those shown in Table S3 (Gaublomme 2015) or listed in Table 4 of WO/2014/134351, incorporated herein by reference; alone or with those of other herein disclosed methods.

In some embodiments, the target gene is one or more Th17-associated transcription regulator(s) selected from those shown in Table S3 (Gaublomme 2015) or Table 5 of WO/2014/134351, incorporated herein by reference; alone or with those of other herein disclosed methods. In some embodiments, the target gene is one or more Th17-associated receptor molecule(s) selected from those listed in Table 6 of WO/2014/134351, incorporated herein by reference; alone or with those of other herein disclosed methods. In some embodiments, the target gene is one or more Th17-associated kinase(s) selected from those listed in Table 7 of WO/2014/134351, incorporated herein by reference; alone or with those of other herein disclosed methods. In some embodiments, the target gene is one or more Th17-associated signaling molecule(s) selected from those listed in Table 8 of WO/2014/134351, incorporated herein by reference; alone or with those of other herein disclosed methods. In some embodiments, the target gene is one or more Th17-associated receptor molecule(s) selected from those listed in Table 9 of WO/2014/134351, incorporated herein by reference; alone or with those of other herein disclosed methods. In some embodiments, the target gene is one or more target genes involved in induction of Th17 differentiation such as, for example one or more of the target genes listed in Table 1 herein or Table 5 of WO/2014/134351, incorporated herein by reference (alone or with those of other herein disclosed methods), as being associated with the early stage of Th17 differentiation, maintenance and/or function. In some embodiments, the target gene is one or more target genes involved in onset of Th17 phenotype and amplification of Th17 T cells such as, for example, one or more of the target genes listed in Table 1 herein or Table 5 of WO/2014/134351, incorporated herein by reference (alone or with those of other herein disclosed methods), as being associated with the intermediate stage of Th17 differentiation, maintenance and/or function. In some embodiments, the target gene is one or more target genes involved in stabilization of Th17 cells and/or modulating Th17-associated interleukin 23 (IL-23) signaling such as, for example, one or more of the target genes listed in Table 1 herein or Table 5 of WO/2014/134351, incorporated herein by reference (alone or with those of other herein disclosed methods), as being associated with the late stage of Th17 differentiation, maintenance and/or function. In some embodiments, the target gene is one or more of the target genes listed in Table 6 of WO/2014/134351, incorporated herein by reference (alone or with those of other herein disclosed methods), as being associated with the early stage of Th17 differentiation. In some embodiments, the target gene is one or more of the target genes listed in Table 6 of WO/2014/134351, incorporated herein by reference (alone or with those of other herein disclosed methods), as being associated with the intermediate stage of Th17 differentiation, maintenance and/or function. In some embodiments, the target gene is one or more of the target genes listed in Table 6 of WO/2014/134351, incorporated herein by reference (alone or with those of other herein disclosed methods), as being associated with the late stage of Th17 differentiation, maintenance and/or function. In some embodiments, the target gene is one or more of the target genes listed in Table 7 of US Pat. App. Pub. 2019/0255107 herein or Table 7 of WO/2014/134351, incorporated herein by reference (alone or with those of other herein disclosed methods), as being associated with the early stage of Th17 differentiation, maintenance and/or function. In some embodiments, the target gene is one or more of the target genes listed in Table 7 of WO/2014/134351, incorporated herein by reference (alone or with those of other herein disclosed methods), as being associated with the intermediate stage of Th17 differentiation, maintenance and/or function. In some embodiments, the target gene is one or more of the target genes listed in Table 7 of WO/2014/134351, incorporated herein by reference (alone or with those of other herein disclosed methods), as being associated with the late stage of Th17 differentiation, maintenance and/or function. In some embodiments, the target gene is one or more of the target genes listed in Table 8 of WO/2014/134351, incorporated herein by reference (alone or with those of other herein disclosed methods), as being associated with the early stage of Th17 differentiation, maintenance and/or function. In some embodiments, the target gene is one or more of the target genes listed in Table 8 of WO/2014/134351, incorporated herein by reference (alone or with those of other herein disclosed methods), as being associated with the intermediate stage of Th17 differentiation, maintenance and/or function. In some embodiments, the target gene is one or more of the target genes listed in Table 8 of WO/2014/134351, incorporated herein by reference (alone or with those of other herein disclosed methods), as being associated with the late stage of Th17 differentiation, maintenance and/or function. In some embodiments, the target gene is one or more of the target genes listed in Table 9 of WO/2014/134351, incorporated herein by reference (alone or with those of other herein disclosed methods), as being associated with the early stage of Th17 differentiation, maintenance and/or function. In some embodiments, the target gene is one or more of the target genes listed in Table S6 (Gaublomme 2015), Table 7 or in Table 9 of WO/2014/134351, incorporated herein by reference (alone or with those of other herein disclosed methods), as being associated with the intermediate stage of Th17 differentiation, maintenance and/or function. In some embodiments, the target gene is one or more of the target genes listed in Table S6 (Gaublomme 2015), Table 7 or Table 9 of WO/2014/134351, incorporated herein by reference (alone or with those of other herein disclosed methods), as being associated with the late stage of Th17 differentiation, maintenance and/or function.

In some embodiments, the target gene is one or more target genes that is a promoter of Th17 cell differentiation. In some embodiments, the target gene is GPR65. In some embodiments, the target gene is also a promoter of pathogenic Th17 cell differentiation and is selected from the group consisting of CD5L, DEC1, PLZP and TCF4.

In some embodiments, the target gene is one or more target genes that is a promoter of pathogenic Th17 cell differentiation. In some embodiments, the target gene is selected from the group consisting of CD5L, DEC1, PLZP and TCF4.

The desired gene or combination of target genes is selected, and after determining whether the selected target gene(s) is overexpressed or under-expressed during Th17 differentiation and/or Th17 maintenance, a suitable antagonist or agonist is used depending on the desired differentiation, maintenance and/or function outcome. For example, for target genes that are identified as positive regulators of Th17 differentiation, use of an antagonist that interacts with those target genes will shift differentiation away from the Th17 phenotype, while use of an agonist that interacts with those target genes will shift differentiation toward the Th17 phenotype. For target genes that are identified as negative regulators of Th17 differentiation, use of an antagonist that interacts with those target genes will shift differentiation toward from the Th17 phenotype, while use of an agonist that interacts with those target genes will shift differentiation away the Th17 phenotype. For example, for target genes that are identified as positive regulators of Th17 maintenance, use of an antagonist that interacts with those target genes will reduce the number of cells with the Th17 phenotype, while use of an agonist that interacts with those target genes will increase the number of cells with the Th17 phenotype. For target genes that are identified as negative regulators of Th17 differentiation, use of an antagonist that interacts with those target genes will increase the number of cells with the Th17 phenotype, while use of an agonist that interacts with those target genes will reduce the number of cells with the Th17 phenotype. Suitable T cell modulating agents include an antibody, a soluble polypeptide, a polypeptide agent, a peptide agent, a nucleic acid agent, a nucleic acid ligand, or a small molecule agent.

In some embodiments, the positive regulator of Th17 differentiation is a target gene selected from MINA, TRPS1, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3, and combinations thereof. In some embodiments, the positive regulator of Th17 differentiation is a target gene selected from MINA, PML, POU2AF1, PROCR, SMARCA4, ZEB1, EGR2, CCR6, FAS and combinations thereof.

In some embodiments, the negative regulator of Th17 differentiation is a target gene selected from SP4, ETS2, IKZF4, TSC22D3, IRF1 and combinations thereof. In some embodiments, the negative regulator of Th17 differentiation is a target gene selected from SP4, IKZF4, TSC22D3 and combinations thereof.

In some embodiments, the T cell modulating agent is a soluble Fas polypeptide or a polypeptide derived from FAS. In some embodiments, the T cell modulating agent is an agent that enhances or otherwise increases the expression, activity, and/or function of FAS in Th17 cells. As shown herein, expression of FAS in T cell populations induced or otherwise influenced differentiation toward Th17 cells. In some embodiments, these T cell modulating agents are useful in the treatment of an immune response, for example, an autoimmune response or an inflammatory response. In some embodiments, these T cell modulating agents are useful in the treatment of an infectious disease or other pathogen-based disorders. In some embodiments, the T cell modulating agent is an antibody, a soluble polypeptide, a polypeptide agonist, a peptide agonist, a nucleic acid agonist, a nucleic acid ligand, or a small molecule agonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells. In some embodiments, the T cell modulating agent is an agent that inhibits the expression, activity and/or function of FAS. Inhibition of FAS expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, these T cell modulating agents are useful in the treatment of autoimmune diseases such as psoriasis, inflammatory bowel disease (IBD), ankylosing spondylitis, multiple sclerosis, Sjögren's syndrome, uveitis, and rheumatoid arthritis, asthma, systemic lupus erythematosus, transplant rejection including allograft rejection, and combinations thereof. In addition, enhancement of Th17 cells is also useful for clearing fungal infections and extracellular pathogens. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells that express additional cytokines. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of CCR5. Inhibition of CCR5 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an inhibitor or neutralizing agent. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of CCR6. Inhibition of CCR6 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of EGR1. Inhibition of EGR1 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

In some embodiments, the T cell modulating agent is an agent that inhibits the expression, activity and/or function of EGR2. Inhibition of EGR2 expression, activity and/or function in T cell populations repressed or otherwise influenced differentiation away from Th17 cells and/or induced or otherwise influenced differentiation toward regulatory T cells (Tregs) and towards Th1 cells. In some embodiments, these T cell modulating agents are useful in the treatment of an immune response, for example, an autoimmune response or an inflammatory response. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cells are naïve T cells. In some embodiments, the T cells are differentiated T cells. In some embodiments, the T cells are partially differentiated T cells. In some embodiments, the T cells are a mixture of naïve T cells and differentiated T cells. In some embodiments, the T cells are mixture of naïve T cells and partially differentiated T cells. In some embodiments, the T cells are mixture of partially differentiated T cells and differentiated T cells. In some embodiments, the T cells are mixture of naïve T cells, partially differentiated T cells, and differentiated T cells.

For example, in some embodiments, the invention provides T cell modulating agents and methods of using these T cell modulating agents to regulate, influence or otherwise impact the phenotype of a Th17 cell or population of cells, for example, by influencing a naïve T cell or population of cells to differentiate to a pathogenic or non-pathogenic Th17 cell or population of cells, by causing a pathogenic Th17 cell or population of cells to switch to a non-pathogenic Th17 cell or population of T cells (e.g., populations of naïve T cells, partially differentiated T cells, differentiated T cells and combinations thereof), or by causing a non-pathogenic Th17 cell or population of T cells (e.g., populations of naïve T cells, partially differentiated T cells, differentiated T cells and combinations thereof) to switch to a pathogenic Th17 cell or population of cells.

In some embodiments, the invention comprises a method of drug discovery for the treatment of a disease or condition involving an immune response involving T cell balance in a population of cells or tissue of a target gene comprising the steps of providing a compound or plurality of compounds to be screened for their efficacy in the treatment of said disease or condition, contacting said compound or plurality of compounds with said population of cells or tissue, detecting a first level of expression, activity and/or function of a target gene, comparing the detected level to a control of level of a target gene, and evaluating the difference between the detected level and the control level to determine the immune response elicited by said compound or plurality of compounds. For example, the method contemplates comparing tissue samples which can be inter alia infected tissue, inflamed tissue, healthy tissue, or combinations of tissue samples thereof.

In one embodiment of the invention, the reductase null animals of the present invention may advantageously be used to modulate T cell balance in a tissue or cell specific manner. Such animals may be used for the applications hereinbefore described, where the role of T cell balance in product/drug metabolism, detoxification, normal homeostasis or in disease etiology is to be studied. It is envisaged that this embodiment will also allow other effects, such as drug transporter-mediated effects, to be studied in those tissues or cells in the absence of metabolism, e.g., carbon metabolism. Accordingly, the animals of the present invention, in a further aspect of the invention may be used to modulate the functions and antibodies in any of the above cell types to generate a disease model or a model for product/drug discovery or a model to verify or assess functions of T cell balance.

In another embodiment, the method contemplates use of animal tissues and/or a population of cells derived therefrom of the present invention as an in vitro assay for the study of any one or more of the following events/parameters: (i) role of transporters in product uptake and efflux; (ii) identification of product metabolites produced by T cells; (iii) evaluate whether candidate products are T cells; or (iv) assess drug/drug interactions due to T cell balance.

The terms “pathogenic” or “non-pathogenic” as used herein are not to be construed as implying that one Th17 cell phenotype is more desirable than the other. As described herein, there are instances in which inhibiting the induction of pathogenic Th17 cells or modulating the Th17 phenotype towards the non-pathogenic Th17 phenotype is desirable. Likewise, there are instances where inhibiting the induction of non-pathogenic Th17 cells or modulating the Th17 phenotype towards the pathogenic Th17 phenotype is desirable.

As used herein, terms such as “pathogenic Th17 cell” and/or “pathogenic Th17 phenotype” and all grammatical variations thereof refer to Th17 cells that, when induced in the presence of TGF-β3, express an elevated level of one or more genes selected from Cxcl3, IL22, IL3, Ccl4, Gzmb, Lrmp, Cc15, Casp1, Csf2, Ccl3, Tbx21, Icos, IL17r, Stat4, Lgals3 and Lag, as compared to the level of expression in a TGF-β3-induced Th17 cells. As used herein, terms such as “non-pathogenic Th17 cell” and/or “non-pathogenic Th17 phenotype” and all grammatical variations thereof refer to Th17 cells that, when induced in the presence of TGF-β3, express a decreased level of one or more genes selected from IL6st, IL1rn, Ikzf3, Maf, Ahr, IL9 and IL10, as compared to the level of expression in a TGF-β3-induced Th17 cells.

In some embodiments, the T cell modulating agent is an agent that enhances or otherwise increases the expression, activity and/or function of Protein C Receptor (PROCR, also called EPCR or CD201) in Th17 cells. As shown herein, expression of PROCR in Th17 cells reduced the pathogenicity of the Th17 cells, for example, by switching Th17 cells from a pathogenic to non-pathogenic signature. Thus, PROCR and/or these agonists of PROCR are useful in the treatment of a variety of indications, particularly in the treatment of aberrant immune response, for example in autoimmune diseases and/or inflammatory disorders. In some embodiments, the T cell modulating agent is an antibody, a soluble polypeptide, a polypeptide agonist, a peptide agonist, a nucleic acid agonist, a nucleic acid ligand, or a small molecule agonist.

In some embodiments, the T cell modulating agent is an agent that inhibits the expression, activity and/or function of the Protein C Receptor (PROCR, also called EPCR or CD201). Inhibition of PROCR expression, activity and/or function in Th17 cells switches non-pathogenic Th17 cells to pathogenic Th17 cells. Thus, these PROCR antagonists are useful in the treatment of a variety of indications, for example, infectious disease and/or other pathogen-based disorders. In some embodiments, the T cell modulating agent is an antibody, a soluble polypeptide, a polypeptide antagonist, a peptide antagonist, a nucleic acid antagonist, a nucleic acid ligand, or a small molecule antagonist. In some embodiments, the T cell modulating agent is a soluble Protein C Receptor (PROCR, also called EPCR or CD201) polypeptide or a polypeptide derived from PROCR. In some embodiments, the invention provides a method of inhibiting Th17 differentiation, maintenance and/or function in a cell population and/or increasing expression, activity and/or function of one or more non-Th17-associated cytokines, one or more non-Th17 associated receptor molecules, or non-Th17-associated transcription regulators selected from FOXP3, interferon gamma (IFN-γ), GATA3, STAT4 and TBX21, comprising contacting a T cell with an agent that inhibits expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent inhibits expression, activity and/or function of at least one of MINA, PML, POU2AF1, PROCR, SMARCA4, ZEB1, EGR2, CCR6, FAS or combinations thereof. In some embodiments, the agent is an antibody, a soluble polypeptide, a polypeptide antagonist, a peptide antagonist, a nucleic acid antagonist, a nucleic acid ligand, or a small molecule antagonist. In some embodiments, the antibody is a monoclonal antibody. In some embodiments, the antibody is a chimeric, humanized or fully human monoclonal antibody. In some embodiments, the T cell is a naïve T cell, and wherein the agent is administered in an amount that is sufficient to modulate the phenotype of the T cell to become and/or produce a desired non-Th17 T cell phenotype, for example, a regulatory T cell (Treg) phenotype or another CD4+ T cell phenotype. In some embodiments, the T cell is a partially differentiated T cell, and wherein the agent is administered in an amount that is sufficient to modulate the phenotype of the partially differentiated T cell to become and/or produce a desired non-Th17 T cell phenotype, for example, a regulatory T cell (Treg) phenotype or another CD4+ T cell phenotype. In some embodiments, the T cell is a Th17 T cell, and wherein the agent is administered in an amount that is sufficient to modulate the phenotype of the Th17 T cell to become and/or produce a CD4+ T cell phenotype other than a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of inhibiting Th17 differentiation in a cell population and/or increasing expression, activity and/or function of one or more non-Th17-associated cytokines, one or more non-Th17-associated receptor molecules, or non-Th17-associated transcription factor selected from FOXP3, interferon gamma (IFN-γ), GATA3, STAT4 and TBX21, comprising contacting a T cell with an agent that enhances expression, activity and/or function of SP4, ETS2, IKZF4, TSC22D3, IRF1 or combinations thereof. In some embodiments, the agent enhances expression, activity and/or function of at least one of SP4, IKZF4, TSC22D3 or combinations thereof. In some embodiments, the agent is an antibody, a soluble polypeptide, a polypeptide agonist, a peptide agonist, a nucleic acid agonist, a nucleic acid ligand, or a small molecule agonist. In some embodiments, the antibody is a monoclonal antibody. In some embodiments, the T cell is a naïve T cell, and wherein the agent is administered in an amount that is sufficient to modulate the phenotype of the T cell to become and/or produce a desired non-Th17 T cell phenotype, for example, a regulatory T cell (Treg) phenotype or another CD4+ T cell phenotype. In some embodiments, the T cell is a partially differentiated T cell, and wherein the agent is administered in an amount that is sufficient to modulate the phenotype of the partially differentiated T cell to become and/or produce a desired non-Th17 T cell phenotype, for example, a regulatory T cell (Treg) phenotype or another CD4+ T cell phenotype. In some embodiments, the T cell is a Th17 T cell, and wherein the agent is administered in an amount that is sufficient to modulate the phenotype of the Th17 T cell to become and/or produce a CD4+ T cell phenotype other than a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of enhancing Th17 differentiation in a cell population increasing expression, activity and/or function of one or more Th17-associated cytokines, one or more Th17-associated receptor molecules, or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines, one or more Th17-associated receptor molecules, or one or more non-Th17-associated transcription regulators selected from FOXP3, interferon gamma (IFN-γ), GATA3, STAT4 and TBX21, comprising contacting a T cell with an agent that inhibits expression, activity and/or function of SP4, ETS2, IKZF4, TSC22D3, IRF1 or combinations thereof. In some embodiments, the agent inhibits expression, activity and/or function of at least one of SP4, IKZF4, TSC22D3 or combinations thereof. In some embodiments, the agent is an antibody, a soluble polypeptide, a polypeptide antagonist, a peptide antagonist, a nucleic acid antagonist, a nucleic acid ligand, or a small molecule antagonist. In some embodiments, the antibody is a monoclonal antibody. In some embodiments, the antibody is a chimeric, humanized or fully human monoclonal antibody. In some embodiments, the T cell is a naïve T cell, and wherein the agent is administered in an amount that is sufficient to modulate the phenotype of the T cell to become and/or produce a desired Th17 T cell phenotype. In some embodiments, the T cell is a partially differentiated T cell, and wherein the agent is administered in an amount that is sufficient to modulate the phenotype of the partially differentiated T cell to become and/or produce a desired Th17 T cell phenotype. In some embodiments, the T cell is a CD4+ T cell other than a Th17 T cell, and wherein the agent is administered in an amount that is sufficient to modulate the phenotype of the non-Th17 T cell to become and/or produce a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of enhancing Th17 differentiation in a cell population, increasing expression, activity and/or function of one or more Th17-associated cytokines, one or more Th17-associated receptor molecules, and/or one or more Th17-associated transcription regulators selected from interleukin 17F (IL-17F), interleukin 17A (IL-17A), STAT3, interleukin 21 (IL-21) and RAR-related orphan receptor C (RORC), and/or decreasing expression, activity and/or function of one or more non-Th17-associated cytokines, one or more Th17-associated receptor molecules, or one or more non-Th17-associated transcription regulators selected from FOXP3, interferon gamma (IFN-γ), GATA3, STAT4 and TBX21, comprising contacting a T cell with an agent that enhances expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent enhances expression, activity and/or function of at least one of MINA, PML, POU2AF1, PROCR, SMARCA4, ZEB1, EGR2, CCR6, FAS or combinations thereof. In some embodiments, the agent is an antibody, a soluble polypeptide, a polypeptide agonist, a peptide agonist, a nucleic acid agonist, a nucleic acid ligand, or a small molecule agonist. In some embodiments, the antibody is a monoclonal antibody. In some embodiments, the antibody is a chimeric, humanized or fully human monoclonal antibody. In some embodiments, the agent is administered in an amount sufficient to inhibit Foxp3, IFN-γ, GATA3, STAT4 and/or TBX21 expression, activity and/or function. In some embodiments, the T cell is a naïve T cell, and wherein the agent is administered in an amount that is sufficient to modulate the phenotype of the T cell to become and/or produce a desired Th17 T cell phenotype. In some embodiments, the T cell is a partially differentiated T cell, and wherein the agent is administered in an amount that is sufficient to modulate the phenotype of the partially differentiated T cell to become and/or produce a desired Th17 T cell phenotype. In some embodiments, the T cell is a CD4+ T cell other than a Th17 T cell, and wherein the agent is administered in an amount that is sufficient to modulate the phenotype of the non-Th17 T cell to become and/or produce a Th17 T cell phenotype. In some embodiments, the T cell is a Th17 T cell, and wherein the agent is administered in an amount that is sufficient to modulate the phenotype of the Th17 T cell to become and/or produce a shift in the Th17 T cell phenotype, e.g., between pathogenic or non-pathogenic Th17 cell phenotype.

In some embodiments, the invention provides a method of identifying genes or genetic elements associated with Th17 differentiation comprising: a) contacting a T cell with an inhibitor of Th17 differentiation or an agent that enhances Th17 differentiation; and b) identifying a gene or genetic element whose expression is modulated by step (a). In some embodiments, the method also comprises c) perturbing expression of the gene or genetic element identified in step b) in a T cell that has been in contact with an inhibitor of Th17 differentiation or an agent that enhances Th17 differentiation; and d) identifying a gene whose expression is modulated by step c). In some embodiments, the inhibitor of Th17 differentiation is an agent that inhibits the expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent inhibits expression, activity and/or function of at least one of MINA, PML, POU2AF1, PROCR, SMARCA4, ZEB1, EGR2, CCR6, FAS or combinations thereof. In some embodiments, the inhibitor of Th17 differentiation is an agent that enhances expression, activity and/or function of SP4, ETS2, IKZF4, TSC22D3, IRF1 or combinations thereof. In some embodiments, the agent enhances expression, activity and/or function of at least one of SP4, IKZF4 or TSC22D3. In some embodiments, the agent that enhances Th17 differentiation is an agent that inhibits expression, activity and/or function of SP4, ETS2, IKZF4, TSC22D3, IRF1 or combinations thereof. In some embodiments, wherein the agent that enhances Th17 differentiation is an agent that enhances expression, activity and/or function of MINA, MYC, NKFB1, NOTCH, PML, POU2AF1, PROCR, RBPJ, SMARCA4, ZEB1, BATF, CCR5, CCR6, EGR1, EGR2, ETV6, FAS, IL12RB1, IL17RA, IL21R, IRF4, IRF8, ITGA3 or combinations thereof. In some embodiments, the agent is an antibody, a soluble polypeptide, a polypeptide antagonist, a peptide antagonist, a nucleic acid antagonist, a nucleic acid ligand, or a small molecule antagonist.

In some embodiments, the invention provides a method of modulating induction of Th17 differentiation comprising contacting a T cell with an agent that modulates expression, activity and/or function of one or more target genes or one or more products of one or more target genes selected from IRF1, IRF8, IRF9, STAT2, STAT3, IRF7, STAT1, ZFP281, IFI35, REL, TBX21, FLI1, BATF, IRF4, one or more of the target genes listed in Table 1 herein or Table 5 of WO/2014/134351, incorporated herein by reference (alone or with those of other herein disclosed methods), as being associated with the early stage of Th17 differentiation, maintenance and/or function, e.g., AES, AHR, ARID5A, BATF, BCL11B, BCL3, CBFB, CBX4, CHD7, CITED2, CREB1, E2F4, EGR1, EGR2, ELL2, ETS1, ETS2, ETV6, EZH1, FLI1, FOXO1, GATA3, GATAD2B, HIF1A, ID2, IFI35, IKZF4, IRF1, IRF2, IRF3, IRF4, IRF7, IRF9, JMJD1C, JUN, LEF1, LRRFIP1, MAX, NCOA3, NFE2L2, NFIL3, NFKB1, NMI, NOTCH1, NR3C1, PHF21A, PML, PRDM1, REL, RELA, RUNX1, SAP18, SATB1, SMAD2, SMARCA4, SP100, SP4, STAT1, STAT2, STAT3, STAT4, STAT5B, STAT6, TFEB, TP53, TRIM24, and/or ZFP161, or any combination thereof.

In some embodiments, the invention provides a method of modulating onset of Th17 phenotype and amplification of Th17 T cells comprising contacting a T cell with an agent that modulates expression, activity and/or function of one or more target genes or one or more products of one or more target genes selected from one or more of the target genes listed in Table 1 herein or Table 5 of WO/2014/134351, incorporated herein by reference (alone or with those of other herein disclosed methods), as being associated with the intermediate stage of Th17 differentiation, maintenance and/or function. In some embodiments, the invention provides a method of modulating stabilization of Th17 cells and/or modulating Th17-associated interleukin 23 (IL-23) signaling comprising contacting a T cell with an agent that modulates expression, activity and/or function of one or more target genes or one or more products of one or more target genes selected from one or more of the target genes listed in Table 1 herein or Table 5 of WO/2014/134351 (alone or with those of other herein disclosed methods), incorporated herein by reference, as being associated with the late stage of Th17 differentiation, maintenance and/or function. In some embodiments, the invention provides a method of modulating one or more of the target genes listed in Table S6 (Gaublomme 2015), Table 7 of US Pat. App. Pub. 2019/0255107 or in Table 6 of WO/2014/134351, incorporated herein by reference (alone or with those of other herein disclosed methods), as being associated with the early stage of Th17 differentiation, maintenance and/or function. In some embodiments, the invention provides a method of modulating one or more of the target genes listed in Table S6 (Gaublomme 2015), Table 7 of US Pat. App. Pub. 2019/0255107 or Table 6 of WO/2014/134351, incorporated herein by reference (alone or with those of other herein disclosed methods), as being associated with the intermediate stage of Th17 differentiation, maintenance and/or function. In some embodiments, the invention provides a method of modulating one or more of the target genes listed in Table S6 (Gaublomme 2015), Table 7 of US Pat. App. Pub. 2019/0255107 or Table 6 of WO/2014/134351 (alone or with those of other herein disclosed methods), incorporated herein by reference, as being associated with the late stage of Th17 differentiation, maintenance and/or function. In some embodiments, the invention provides a method of modulating one or more of the target genes listed in Table 7 of WO/2014/134351 (alone or with those of other herein disclosed methods), incorporated herein by reference, as being associated with the early stage of Th17 differentiation, maintenance and/or function. In some embodiments, the invention provides a method of modulating one or more of the target genes listed in Table 7 of WO/2014/134351, incorporated herein by reference (alone or with those of other herein disclosed methods), asc being associated with the intermediate stage of Th17 differentiation, maintenance and/or function. In some embodiments, the invention provides a method of modulating one or more of the target genes listed in Table 7 of WO/2014/134351, incorporated herein by reference (alone or with those of other herein disclosed methods), as being associated with the late stage of Th17 differentiation, maintenance and/or function. In some embodiments, the invention provides a method of modulating is one or more of the target genes listed in Table 8 of WO/2014/134351, incorporated herein by reference (alone or with those of other herein disclosed methods), as being associated with the early stage of Th17 differentiation, maintenance and/or function. In some embodiments, the invention provides a method of modulating one or more of the target genes listed in Table 8 of WO/2014/134351, incorporated herein by reference (alone or with those of other herein disclosed methods), as being associated with the intermediate stage of Th17 differentiation, maintenance and/or function. In some embodiments, the invention provides a method of modulating one or more of the target genes listed in Table 8 of WO/2014/134351, incorporated herein by reference (alone or with those of other herein disclosed methods), as being associated with the late stage of Th17 differentiation, maintenance and/or function. In some embodiments, the invention provides a method of modulating one or more of the target genes listed in Table 9 of WO/2014/134351, incorporated herein by reference (alone or with those of other herein disclosed methods), as being associated with the early stage of Th17 differentiation, maintenance and/or function. In some embodiments, the invention provides a method of modulating one or more of the target genes listed in Table 9 of WO/2014/134351, incorporated herein by reference (alone or with those of other herein disclosed methods), as being associated with the intermediate stage of Th17 differentiation, maintenance and/or function. In some embodiments, the invention provides a method of modulating one or more of the target genes listed in Table 9 of WO/2014/134351, incorporated herein by reference (alone or with those of other herein disclosed methods), as being associated with the late stage of Th17 differentiation, maintenance and/or function. In some embodiments, the invention provides a method of inhibiting tumor growth in a subject in need thereof by administering to the subject a therapeutically effective amount of an inhibitor of Protein C Receptor (PROCR). In some embodiments, the inhibitor of PROCR is an antibody, a soluble polypeptide, a polypeptide agent, a peptide agent, a nucleic acid agent, a nucleic acid ligand, or a small molecule agent. In some embodiments, the inhibitor of PROCR is one or more agents selected from the group consisting of lipopolysaccharide; cisplatin; fibrinogen; 1, 10-phenanthroline; 5-N-ethylcarboxamido adenosine; cystathionine; hirudin; phospholipid; Drotrecogin alfa; VEGF; Phosphatidylethanolamine; serine; gamma-carboxyglutamic acid; calcium; warfarin; endotoxin; curcumin; lipid; and nitric oxide.

In some embodiments, the invention provides a method of diagnosing an immune response in a subject, comprising detecting a level of expression, activity and/or function of one or more signature genes or one or more products of one or more signature genes selected from those listed in Table 1 or 2 of WO/2014/134351, incorporated herein by reference (alone or with those of other herein disclosed methods), and comparing the detected level to a control of level of signature gene or gene product expression, activity and/or function, wherein a difference between the detected level and the control level indicates that the presence of an immune response in the subject. In some embodiments, the immune response is an autoimmune response. In some embodiments, the immune response is an inflammatory response, including inflammatory response(s) associated with an autoimmune response and/or inflammatory response(s) associated with an infectious disease or other pathogen-based disorder.

In some embodiments, the invention provides a method of monitoring an immune response in a subject, comprising detecting a level of expression, activity and/or function of one or more signature genes or one or more products of one or more signature genes, e.g., one or more signature genes selected from those listed in Table 1 or 2 of WO/2014/134351, incorporated herein by reference (alone or with those of other herein disclosed methods), at a first time point, detecting a level of expression, activity and/or function of one or more signature genes or one or more products of one or more signature genes, e.g., one or more signature genes selected from those listed in Table 1 or 2 of WO/2014/134351 (alone or with those of other herein disclosed methods), incorporated herein by reference, at a second time point, and comparing the first detected level of expression, activity and/or function with the second detected level of expression, activity and/or function, wherein a change between the first and second detected levels indicates a change in the immune response in the subject. In some embodiments, the immune response is an autoimmune response. In some embodiments, the immune response is an inflammatory response.

In some embodiments, the invention provides a method of monitoring an immune response in a subject, comprising isolating a population of T cells from the subject at a first time point, determining a first ratio of T cell subtypes within the T cell population at a first time point, isolating a population of T cells from the subject at a second time point, determining a second ratio of T cell subtypes within the T cell population at a second time point, and comparing the first and second ratio of T cell subtypes, wherein a change in the first and second detected ratios indicates a change in the immune response in the subject. In some embodiments, the immune response is an autoimmune response. In some embodiments, the immune response is an inflammatory response.

In some embodiments, the invention provides a method of activating therapeutic immunity by exploiting the blockade of immune checkpoints. The progression of a productive immune response requires that a number of immunological checkpoints be passed. Immunity response is regulated by the counterbalancing of stimulatory and inhibitory signal. The immunoglobulin superfamily occupies a central importance in this coordination of immune responses, and the CD28/cytotoxic T-lymphocyte antigen-4 (CTLA-4):B7.1/B7.2 receptor/ligand grouping represents the archetypal example of these immune regulators (see e.g., Korman A J, Peggs K S, Allison J P, “Checkpoint blockade in cancer immunotherapy.” Adv Immunol. 2006; 90:297-339). In part the role of these checkpoints is to guard against the possibility of unwanted and harmful self-directed activities. While this is a necessary function aiding in the prevention of autoimmunity, it may act as a barrier to successful immunotherapies aimed at targeting malignant self-cells that largely display the same array of surface molecules as the cells from which they derive. The expression of immune-checkpoint proteins can be dysregulated in a disease or disorder and can be an important immune resistance mechanism. Therapies aimed at overcoming these mechanisms of peripheral tolerance, in particular by blocking the inhibitory checkpoints, offer the potential to generate therapeutic activity, either as monotherapies or in synergism with other therapies.

Thus, the present invention relates to a method of engineering T-cells, especially for immunotherapy, comprising modulating T cell balance to inactivate or otherwise inhibit at least one gene or gene product involved in the immune check-point.

Suitable T cell modulating agent(s) for use in any of the compositions and methods provided herein include an antibody, a soluble polypeptide, a polypeptide agent, a peptide agent, a nucleic acid agent, a nucleic acid ligand, or a small molecule agent. By way of non-limiting example, suitable T cell modulating agents or agents for use in combination with one or more T cell modulating agents are shown in Table 10 of WO/2014/134351, incorporated herein by reference (alone or with those of other herein disclosed methods), of the specification.

One skilled in the art will appreciate that the T cell modulating agents have a variety of uses. For example, the T cell modulating agents are used as therapeutic agents as described herein. The T cell modulating agents can be used as reagents in screening assays, diagnostic kits or as diagnostic tools, or these T cell modulating agents can be used in competition assays to generate therapeutic reagents.

In some embodiments, the invention provides a method of diagnosing, prognosing and/or staging an immune response involving Th17 T cell balance, comprising detecting a first level of expression of one or more of saturated fatty acids (SFA) and/or polyunsaturated fatty acids (PUFA) in Th17 cells, and comparing the detected level to a control level of saturated fatty acids (SFA) and/or polyunsaturated fatty acids (PUFA), wherein a change in the first level of expression and the control level detected indicates a change in the immune response in the subject. In one embodiment, a shift towards polyunsaturated fatty acids (PUFA) and away from saturated fatty acids (SFA) indicates a non-pathogenic Th17 response.

In some embodiments, the invention provides a method for monitoring subjects undergoing a treatment or therapy involving T cell balance comprising, detecting a first level of expression of one or more of saturated fatty acids (SFA) and/or polyunsaturated fatty acids (PUFA) in Th17 cells in the absence of the treatment or therapy and comparing the detected level to a level of saturated fatty acids (SFA) and/or polyunsaturated fatty acids (PUFA) in the presence of the treatment or therapy, wherein a difference in the level of expression in the presence of the treatment or therapy indicates whether the subject is responsive to the treatment or therapy.

In another embodiment, the invention provides a method for monitoring subjects undergoing a treatment or therapy involving T cell balance comprising detecting a first level of expression of one or more of saturated fatty acids (SFA) and polyunsaturated fatty acids (PUFA) in Th17 cells in the absence of the treatment or therapy and comparing the ratio of detected level to a ratio of detected level of saturated fatty acids (SFA) and polyunsaturated fatty acids (PUFA) in the presence of the treatment or therapy, wherein a shift in the ratio in the presence of the treatment or therapy indicates whether the subject is responsive to the treatment or therapy. Not being bound by a theory, a shift in the ratio towards polyunsaturated fatty acids (PUFA) and away from saturated fatty acids (SFA) indicates a non-pathogenic Th17 response.

In another embodiment, the therapy may be a lipid, preferably a mixture of lipids of the present invention. The lipids may be synthetic. Not being bound by a theory, a treatment comprising lipids may shift T cell balance.

In another embodiment, the treatment or therapy involving T cell balance is for a subject undergoing treatment or therapy for cancer. Not being bound by a theory, shifting Th17 balance towards a pathogenic phenotype would allow a stronger immune response against a tumor.

In some embodiments, the invention provides a method of drug discovery for the treatment of a disease or condition involving an immune response involving Th17 T cell balance in a population of cells or tissue comprising: (a) providing a compound or plurality of compounds to be screened for their efficacy in the treatment of said disease or condition; (b) contacting said compound or plurality of compounds with said population of cells or tissue; (c) detecting a first level of expression of one or more of saturated fatty acids (SFA) and/or polyunsaturated fatty acids (PUFA) in Th17 cells, optionally calculating a ratio; (d) comparing the detected level to a control level of saturated fatty acids (SFA) and/or polyunsaturated fatty acids (PUFA), optionally comparing the shift in ratio; and, (e) evaluating the difference between the detected level and the control level to determine the immune response elicited by said compound or plurality of compounds.

In some embodiments, a panel of lipids is detected. The panel may include saturated fatty acids (SFA) and/or polyunsaturated fatty acids (PUFA) whose expression is changed at least 1.5 fold when comparing wild type Th17 cells to CD5L^{-/-} Th17 cells after treatment with non-pathogenic inducing cytokines. The non-pathogenic inducing cytokines may be TGF-β1+IL-6. The panel may include lipids whose expression is changed upon differentiation into a pathogenic or non-pathogenic Th17 cell. In another embodiment single saturated fatty acids (SFA) and/or polyunsaturated fatty acids (PUFA) representative of lipids whose expression is changed in response to CD5L loss or differentiation are detected. In a preferred embodiment, the SFA is a cholesterol ester or palmitic acid and the PUFA is a PUFA-containing triacylglyceride or arachidonic acid. In one embodiment only a single SFA or PUFA is detected.

In some embodiments, the treatment or therapy is a formulation comprising at least one lipid. The at least one lipid may be a synthetic lipid. Not being bound by a theory an autoimmune disease may be treated with polyunsaturated fatty acids (PUFA) and a disease requiring an enhanced immune response may be treated with saturated fatty acids (SFA).

Pathways Associated with T Cell Activation and T Cell Dysfunction

The various aspects of the invention as disclosed in this specification are based, at least in part, on the novel discovery of useful markers, marker signatures and molecular targets associated with immune cell dysfunction and/or activation. More particularly, certain of the present markers, marker signatures and molecular targets correlate with the loss of effector function of the immune cells and are advantageously distinct, separate or uncoupled from, or independent of the immune cell activation status. Certain other of the present markers, marker signatures and molecular targets correlate with immune cell activation and are advantageously distinct, separate or uncoupled from, or independent of the immune cell dysfunction status.

Previously, obtaining molecular signatures for T cell dysfunction has been complicated by the fact that T cell dysfunction arises from chronic T cell activation, whereby molecular signatures of T cell dysfunction and activation are closely intertwined. Hence, co-inhibitory receptors that mark dysfunctional T cells are also up-regulated during T cell activation, where they function to contract the effector T cell population and restore immune homeostasis. Furthermore, dysfunctional CD8⁺ T cells and activated CD8⁺ T cells both upregulate genes that regulate activation of the cell cycle, T cell homing and migration and effector molecules such as granzymes, and both down-regulate memory cell gene signatures (Wherry et al. 2007, supra; Doering et al. 2012, supra). Indeed, T cell “dysfunction” may have likely evolved as a physiological process to carefully balance T cell activation and self-regulation in the face of chronic antigen persistence, thereby limiting immunopathology and minimizing collateral damage to the host.

An experimental and computational approach was used to systematically dissect the molecular pathways associated with activation and “dysfunction” within CD8⁺ tumor-infiltrating lymphocytes (TILs), allowing to uncouple molecular signatures for T cell dysfunction and activation. The present analysis identifies gene modules that are uniquely associated with the dysfunctional T cell state and activated T cell state, and key molecular nodes that control them. The present markers, marker signatures and molecular targets thus provide for new ways to evaluate and modulate immune responses, such as to specifically evaluate and target the dysfunctional T cell state while leaving T cell activation programs intact.

Accordingly, an aspect of the invention relates to a method of detecting dysfunctional immune cells comprising detection of a gene expression signature comprising one or more markers of dysfunction selected from the group consisting of FOXO1, GATA3, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, ILR5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14.

A related aspect relates to a method of detecting dysfunctional immune cells comprising detection of a gene expression signature comprising one or more markers of dysfunction selected from the group consisting of FOXO1, GATA3, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14. A further aspect of the invention relates to a method of detecting dysfunctional immune cells comprising detection of a gene expression signature comprising one or more markers of dysfunction selected from the group consisting of NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, POU2AF1, GATA3, B3GNT2, FAS, PIAS2, FOXO1, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, and BTLA. A related aspect provides a method of detecting dysfunctional immune cells comprising detection of a gene expression signature comprising one or more markers of dysfunction selected from the group consisting of the markers listed in Table 3 of US Pat. App. Pub. 2019/0100801, part “Dysfunction_module”, Table 5A or Table 5B of US Pat. App. Pub. 2019/0100801.

A further aspect of the invention relates to a method of detecting activated immune cells comprising detection of a gene expression signature comprising one or more markers of activation selected from the group consisting of TMCO1, PRMT5, EXOC4, TYR, HDHD2, RCN1, LMNB2, TCTEX1D2, VMA21, HCFC2, MRPS27, DUSP19, CD200R4, SRSF10, NAP1L4, ZADH2, ERGIC1, STARD3NL, RCC1, CD38, ZFP142, METTL10, MOGS, S100PBP, AREG, 1700052N19RIK, NDUFA13, RFT1, TAF12, ELP2, TONSL, FANCG, PIGF, GNG2, HIST1H1E, MINA, NDUFAB1, AP1M1, DYNLT1C, JAGN1, CERS4, METTL3, GCDH, RBX1, HAUS4, TFIP11, BC026590, PSMB9, PTPN23, PIAS3, TMEM129, DPYSL2, TMEM209, CALU, EXOSC1, PQLC3, ACO1, PDIA4, POLR3K, NTAN1, PSMB3, ARFIP1, PHF11B, MYEF2, TIMM50, ACAD8, RDM1, CCNH, TMEM41A, PLAA, MEAF6, EXOSC3, QRSL1, UPF1, ANXA6, FTSJD2, PRPSAP1, ARSB, GM11127, HNRNPA2B1, NUP35, RPRD1B, NCBP2, HIST1H3E, KIFC1, MLH1, CD200R1, CPSF6, CDT1, PPM1G, MRPS33, PRADC1, GBP3, RAD17, MTHFSD, FOXRED1, TAX1BP3, C1D, TPM3, D16ERTD472E, SARS2, 0610009O20RIK, ARPP19, ASRGL1, SDF2L1, TBCC, MYG1, SEPHS1, DYNC1LI1, ZBTB38, TARDBP, SLC9A8, TYK2, THUMPD3, MRPL16, ACOT8, LRRK1, HMGB1, HSPA1B, TCEA1, MAVS, POFUT2, VPS53, RIT1, SNAPC1, DNAAF2, COMMD10, PMPCB, EHBP1L1, ADAT3, DOHH, LSM4, PTCD1, GMPPB, LAMTOR1, DRG2, CDCA7L, SSBP1, ANAPC15, NAGLU, AKR1B3, PAOX, EIF4E2, GPAA1, RAD50, STX18, GRPEL1, VMP1, REXO2, HIST1H1C, ZFP429, GGH, TAF6, COMMD3, PARL, RBM18, 2700029M09RIK, EXOSC4, ABHD10, DNAJC14, DPCD, ATPBD4, SERPINA3F, CTCF, LMAN1, NEU3, EIF2D, HAUS5, USF1, AAR2, FARSB, COG4, COG2, FKBP2, SLC35A1, DPY30, ALDH3A2, 1110008P14RIK, KLRE1, ZDHHC6, RAD18, TSPAN4, METTL20, NUDT16L1, TMEM167, IPP, INIP, REEP4, ERP44, GIMAP7, CYB5B, ACAT2, ANAPC5, PEX19, PUF60, SLBP, MTG1, ACTR10, CCDC127 and KPNB1.

A further aspect of the invention relates to a method of detecting dysfunctional and/or activated immune cells comprising detection of a gene expression signature comprising one or more markers selected from the group consisting of SEC23A, ACTN4, MTMR1, TIGIT, TRIP13, NCOR2, CCDC50, LPCAT1, GMNN, CCR8, FLNA, CIAPIN1, TK1, E430025E21RIK, ENDOD1, RGS8, SLC35A3, ARL6IP1, CALM3, MCM3, MKI67, SLC25A13, SUOX, AP3S1, NAA38, NUCKS1, CDCA8, UHRF2, RAD54L, PSAT1, FEM1B, MCM5, CCNB2, CX3CR1, SH3BGRL, HIST1H1B, CASP3, DNMT3A, CCNA2, DUT, STMN1, MEMO1, WHSC1, BUB1B, FKBP1A, CCT7, ATP6V1A, POLA1, GTDC1, RPPH1, NR4A2, AP2M1, FUT7, CDCA3, STRN, CHAF1A, IL18RAP, ST14, ADAMTS14, ACTG1, KIF13B, PTPN5, RAB8B, SERPINE2, CSTF2, EIF4H, GM5069, TMEM48, CTLA4, GM9855, EZH2, MMS22L, RAD51, TPX2, METRN, TMEM126A, HIF1A, MSH6, NCAPD2, UHRF1, ALCAM, HMGN2, MAP4, POLD1, DGKZ, LCP1, AURKB, MRPS22, 2810417H13RIK, WDR76, GALNT3, IPO5, GM5177, NAB2, CISH, ARF5, CENPH, STAP1, KIF15, HIST1H2AG, CDC45, PTPN11, GINS1, TFDP1, MLF2, PGP, POLE, HIST1H2AO, IL10RA, LDHA, SERPINB6A, ASNSD1, LCLAT1, CALR, LGALS1, NDFIP2, GPD2, RRM1, TPI1, DUSP14, MAD2L1, MLEC, CRMP1, DTL, PDCD1, INTS7, WDR3, MED14, EEA1, UAP1, FAR1, GAPDH, YWHAH, MMD, CSF1, HN1L, MDFIC, DUSP4, IL2RA, ALDOA, HIST2H3B, ENO1, SIVA1, TNFRSF4, TNFRSF9, CSRP1, IGFBP7, MCM6, RDX, KIF2C, RBL2, BCL2A1B, HIST1H3C, ATP5B, CIT, B4GALT5, HELLS, TRPS1, FAM129A, TXN1, HSP90AB1, H2AFZ, METAP2, DESI1, FIGNL1, LIN54, CAPG, SYNE3, AI836003, LIG1, HCFC1, GARS, SMARCA5, PGK1, PPP2R4, BCL2A1D, PPP1CA, RBPJ, BHLHE40, SLC16A3, DNMT1, S100A4, PKM, PRELID1, KIF20A, ITGAV, TWSG1, TACC3, ATP5F1, RQCD1, ANKRD52, RGS16, ANXA2, TMPO, ATP10A, PRIM1, ZFP207, STX11, RPS2, and TOPBP1.

A further aspect of the invention relates to a method of detecting naïve-memory-like immune cells comprising detection of a gene expression signature comprising one or more markers selected from the group consisting of GPR183, THA1, TREML2, ZNRF3, CDK2AP2, CREB3, RPS16, BLOC1S2A, ATP1B3, BLNK, RPS29, SHARPIN, TSC22D1, KLRA1, HSD11B1, RPS15, AKAP8L, PHC1, RPL31, S1PR1, GM5547, SRSF5, ACSS2, ADK, AMICA1, ATP1B1, CNP, SNHG8, FCRLA, H2-T23, RAB33B, TLR12, RPF1, SP140, SH3GL1, CTSL, RPGRIP1, 5430417L22RIK, CXXC5, RABGGTA, KCNJ8, DYM, FRAT1, SPIB, ADRB2, COX6A2, TMEM219, GPR18, CCPG1, PLCB2, CALM2, KYNU, CRLF3, IDNK, TNFRSF26, DNAJB9, TXNIP, UPB1, GM11346, PHF1, RPL18A, DNTT, HAAO, PIM2, RABAC1, APOPT1, BIN2, OXR1, GPR171, RASGRP2, SLC9A9, 5830411N06RIK, PIAS1, PYDC3, ZCCHC18, TCSTV3, KLRA7, NPC2, CD180, SMIM14, P2RY14, PDLIM1, MYLIP, PDE2A, PPIF, KLRA17, FBXO32, DIRC2, ELOVL6, PJA1, SP110, KLRA6, USP7, HCST, KLRA23, GAB3, TOM1, ACP5, PBLD1, SMPD5, EVI2A, KLF13, MFSD11, IFNGR1, POU6F1, USEl, HDAC4, SMIM5, MAF1, 1810034E14RIK, TSC22D3, GAS5, RPL21, RELL1, SERTAD2, BC147527, KMO, SKAP1, TCF4, SP100, RNF167, TMEM59, IRGM1, CD69, DNAJC7, PIK3IP1, TAZ, HAVCR1, LY6D, RPL23, DAPP1, FLT3, ITM2B, NUCB2, RPS14, GIMAP9, HBP1, MAN2A2, RNF122, SOCS3, CD7, PNCK, 2610019F03RIK, SLC27A1, BPTF, H2-Q9, KLHL6, RPL17, SEMA4B, LDLRAD4, TCEA2, GM14207, CIRBP, FAM189B, ZFP707, ATP10D, RNASET2A, ATP2A1, BST2, EYA2, IRF7, ITPR2, STK17B, CYBASC3, TRIM11, KLK1B27, ZMYND8, LEF1, RNASE6, EIF4A2, HS3ST1, NIPBL, STX4A, UGCG, CAMKlD, PPFIA4, UVRAG, CDKN2D, ZBTB21, LEFTY1, APBB1IP, GIMAP3, H13, RGS10, RNF138, RPL12, SLC7A6OS, FADS2, SELPLG, CXCR4, GPR146, ZFP386, BCL11A, TRIM34A, RPS7, TLR9, PACSIN1, PAIP1, PGAM2 and JAKMIP1.

A yet further aspect of the invention relates to a kit of parts comprising means for detection of the above signature of dysfunction. Also provided is a kit of parts comprising means for detection of the signature of dysfunction, activation, activation and/or dysfunction, or memory as taught herein.

Another aspect of the invention provides a method for determining whether or not an immune cell has a dysfunctional immune phenotype and/or whether or not an immune cell would benefit from upregulation of an immune response, said method comprising: (a) determining in said immune cell the expression of POU2AF1, GATA3 and/or FOXO1, whereby expression of POU2AF1, GATA3 and/or FOXO1 indicates that the immune cell has a dysfunctional immune phenotype and/or would benefit from upregulation of an immune response; or (b) determining in said immune cell the expression of the signature of dysfunction as defined herein, whereby expression of the signature indicates that the immune cell has a dysfunctional immune phenotype and/or would benefit from upregulation of an immune response.

Also provided is a method for determining whether or not an immune cell has an activation, activation and/or dysfunction or memory immune phenotype and/or whether or not an immune cell would benefit from modulation (e.g., downregulation or upregulation) of an immune response, said method comprising determining in said immune cell the expression of the signature of activation, activation and/or dysfunction, or memory, as defined herein, whereby expression of the signature indicates that the immune cell has respectively an activation, activation and/or dysfunction or memory immune phenotype and/or would benefit from modulation of an immune response.

A further aspect of the invention provides a method for determining whether or not a patient would benefit from a therapy aimed at reducing dysfunction of immune cells or a therapy aimed at upregulating of an immune response, the method comprising: (a) determining, in immune cells from said patient the expression of POU2AF1, GATA3 and/or FOXO1, whereby expression of POU2AF1, GATA3 and/or FOXO1 indicates that the patient will benefit from the therapy; or (b) determining, in immune cells from said patient the expression of the signature of dysfunction as defined above, whereby expression of the signature indicates the patient will benefit from the therapy.

Also provided is a method for determining whether or not a patient would benefit from a therapy aimed at modulating (e.g., reducing or increasing) activation, activation and/or dysfunction or memory phenotype of immune cells, or a therapy aimed at modulating (e.g., reducing or increasing) of an immune response, said method comprising determining, in immune cells from said patient the expression of the signature of activation, activation and/or dysfunction, or memory, as defined herein, whereby expression of the signature indicates that the patient will benefit from the therapy aimed at modulating respectively the activation, activation and/or dysfunction or memory phenotype of immune cells, or will benefit from the therapy aimed at modulating the immune response.

Another aspect of the invention relates to a method for determining the efficacy of a treatment of a patient with a therapy, said method comprising: (a) determining in immune cells from said patient the expression of POU2AF1, GATA3 and/or FOXO1 before and after said treatment and determining the efficacy of said therapy based thereon, whereby unchanged or increased expression of POU2AF1, GATA3 and/or FOXO1 indicates that the treatment should be adjusted; or (b) determining in immune cells from said patient the expression of the signature of dysfunction as defined above before and after said treatment and determining the efficacy of said therapy based thereon, whereby unchanged or increased expression of the signature indicates that the treatment should be adjusted.

Also provided is a method for determining the efficacy of a treatment of a patient with a therapy, said method comprising determining in immune cells from said patient the expression of the signature of activation, activation and/or dysfunction, or memory, as defined herein, before and after said treatment and determining the efficacy of said therapy based thereon, whereby unchanged or increased expression of the signature indicates that the treatment should be adjusted.

Another aspect of the invention provides a method for determining the suitability of a compound as a checkpoint inhibitor, said method comprising: (a) contacting an immune cell expressing POU2AF1, GATA3 and/or FOXO1 with said compound and determining whether or not said compound can affect the expression of POU2AF1, GATA3 and/or FOXO1 by said cell, whereby decreased expression indicates that the compound is suitable as a checkpoint inhibitor; or (b) contacting an immune cell expressing the signature of dysfunction as defined above with said compound and determining whether or not said compound can affect the expression of the signature by said cell, whereby decreased expression indicates that the compound is suitable as a checkpoint inhibitor.

Also provided is a method for determining the suitability of a compound as a checkpoint inhibitor, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable as a checkpoint inhibitor (e.g., whereby increased expression of the signature of activation indicates that the compound is suitable as a checkpoint inhibitor).

A further aspect of the invention provides a method for determining the suitability of a compound for reducing a dysfunctional immune phenotype and/or upregulating of an immune response, said method comprising: (a) contacting an immune cell expressing POU2AF1, GATA3 and/or FOXO1 with said compound and determining whether or not said compound can affect the expression of POU2AF1, GATA3 and/or FOXO1 by said cell, whereby decreased expression indicates that the compound is suitable for reducing dysfunctional immune phenotype and/or upregulating of an immune response; or (b) contacting an immune cell expressing the signature of dysfunction as defined above with said compound and determining whether or not said compound can affect the expression of the signature by said cell, whereby decreased expression indicates that the compound is suitable for reducing dysfunctional immune phenotype and/or upregulating of an immune response. Also provided is a method for determining the suitability of a compound for modulating (e.g., reducing or increasing) activation, activation and/or dysfunction or memory phenotype of immune cells, and/or modulating (e.g., reducing or increasing) of an immune response, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable for modulating respectively the activation, activation and/or dysfunction or memory phenotype of immune cells, and/or modulating of the immune response.

A yet another aspect of the invention provides a method for stratification of immune cells into one or more cell populations comprising at least a first cell population having a comparatively more dysfunctional immune phenotype and a second population having a comparatively less dysfunctional immune phenotype, comprising: (a) determining in said immune cells the expression of POU2AF1, GATA3 and/or FOXO1, and allotting cells having no or comparatively lower expression of POU2AF1, GATA3 and/or FOXO1 into said second population, and cells having comparatively higher expression of POU2AF1, GATA3 and/or FOXO1 into said first population; or (b) determining in said immune cells the expression of the signature of dysfunction as defined above, and allotting cells having no or comparatively lower expression of said signature into said second population, and cells having comparatively higher expression of said signature into said first population.

Also provided is a method for stratification of immune cells into one or more cell populations comprising at least a first cell population having a comparatively more activation, activation and/or dysfunction or memory phenotype and a second population having a comparatively less activation, activation and/or dysfunction or memory phenotype, said method comprising determining in said immune cells the expression of the signature of activation, activation and/or dysfunction, or memory, as defined herein, and allotting cells having no or comparatively lower expression of said signature into said second population, and cells having comparatively higher expression of said signature into said first population.

A yet another aspect provides a method of isolating an immune cell as taught herein comprising binding of an affinity ligand to a signature gene expressed on the surface of the immune cell.

A further aspect provides a method of treating a subject in need thereof, comprising administering to said subject an agent capable of modulating the immune cell as taught herein.

A further aspect provides a method of treatment comprising administering one or more checkpoint inhibitors to a patient in need thereof, wherein immune cells obtained from the patient have a gene signature as taught herein, such as the gene signature of dysfunction as taught herein.

A further aspect of the invention provides an isolated immune cell modified to comprise an altered expression or activity of POU2AF1, GATA3 or FOXO1. Further aspects provide an isolated immune cell modified to comprise an altered expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory like_module”. A further aspect provides a cell population of said modified immune cells.

Another aspect relates to a method for generating said modified immune cell, the method comprising (i) providing an isolated immune cell, and (ii) modifying said isolated immune cell such as to comprise an altered expression of POU2AF1, GATA3 or FOXO1. Further aspects provide a method for generating said modified immune cell, the method comprising (i) providing an isolated immune cell, and (ii) modifying said isolated immune cell such as to comprise an altered expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory like module”.

A further aspect of the invention provides an isolated immune cell modified to comprise an agent capable of inducibly altering expression or activity of POU2AF1, GATA3 or FOXO1. Further aspects provide an isolated immune cell modified to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”. A further aspect provides a cell population of said modified immune cells.

Another aspect relates to a method for generating said modified immune cell, the method comprising (i) providing an isolated immune cell, and (ii) modifying said isolated immune cell such as to comprise an agent capable of inducibly altering expression of POU2AF1, GATA3 or FOXO1. Further aspects provide a method for generating said modified immune cell, the method comprising (i) providing an isolated immune cell, and (ii) modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”.

Another aspect of the invention provides a pharmaceutical composition comprising the isolated immune cell or the cell population as defined above.

A further aspect of the invention relates to the isolated immune cell or the cell population as defined above for use in therapy.

Another aspect of the invention provides the isolated immune cell or the cell population as defined above for use in immunotherapy or adoptive immunotherapy.

A further aspect of the invention relates to a method of treating a subject in need thereof, comprising administering to said subject the isolated immune cell or the cell population as defined above.

Another aspect of the invention provides a method of treating a subject in need thereof, comprising: (a) providing an isolated immune cell from the subject, or isolating an immune cell from a subject; (b) modifying said isolated immune cell such as to comprise an altered expression or activity of POU2AF1, GATA3 and/or FOXO1, or modifying said isolated immune cell such as to comprise an agent capable of inducibly altering expression or activity of POU2AF1, GATA3 and/or FOXO1; and (c) reintroducing the modified isolated immune cell to the subject. Further aspects provide a method of treating a subject in need thereof, comprising: (a) providing an isolated immune cell from the subject, or isolating an immune cell from a subject; (b) modifying said isolated immune cell such as to comprise an altered expression or activity of, or modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”; and (c) reintroducing the modified isolated immune cell to the subject.

The method of treatment may be for a condition, disease or disorder where an enhanced immune response is required, such as but not limited to a cancer, or a condition, disease or disorder where a decreased immune response is required, such as but not limited to an autoimmune disease. The immune cell may be modified such that expression of a gene signature is altered. The immune cell may be modified by treatment with an agent specific for downregulating expression or activity of at least one gene of one gene signature. The immune cell may be modified by treatment with an agent specific for upregulating expression or activity in at least one gene of an opposing gene signature. A gene in the dysfunctional gene signature and a gene in the activation signature may be such modified. Not being bound by a theory, cancer may be treated by obtaining a dysfunctional T cell and treating with an agent that activates the cell. Not being bound by a theory, introducing dysfunctional cells to a subject with an autoimmune disease may be performed. Dysfunctional cells secrete suppressive cytokines that may suppress immune cells causing the autoimmunity. A gene, gene signature or immune cell may be modified ex vivo. A gene, gene signature or immune cell may be modified ex vivo. A gene, gene signature or immune cell may be modified in vivo. Not being bound by a theory, modifying immune cells in vivo, such that dysfunctional immune cells are decreased can provide a therapeutic effect by enhancing an immune response in a subject. A gene, gene signature or immune cell may be modified by a small molecule, a DNA targeting agent, or a therapeutic antibody or antibody fragment thereof. As described herein, a DNA targeting agent may be a CRISPR system.

In another aspect, a method of treatment may comprise treating a subject with an agent specific for, e.g., capable of suppressing or activating, a cell type as defined by any one gene signature as taught herein, e.g., any one of the gene signatures, or portions thereof, as set forth in Table 3 of US Pat. App. Pub. 2019/0100801, Table 5A or Table 5B of US Pat. App. Pub. 2019/0100801. In certain embodiments, the agent is capable of suppressing an immune cell defined by any one of the gene signatures set forth in Table 3 of US Pat. App. Pub. 2019/0100801, Table 5A or Table 5B of US Pat. App. Pub. 2019/0100801. In certain other embodiments, the agent is capable of activating an immune cell defined by any one of the gene signatures set forth in Table 3 of US Pat. App. Pub. 2019/0100801, Table 5A or Table 5B of US Pat. App. Pub. 2019/0100801. In a preferred embodiment a dysfunctional T cell is targeted with an agent specific for a gene present only in the dysfunctional gene signature. In another embodiment an activated T cell is targeted with an agent specific for a gene present only in the activation gene signature. The gene may encode a surface protein. The agent may be a drug conjugated antibody. Not being bound by a theory, suppressing, such as by ablating dysfunctional T cells can increase cellular mediated toxicity of remaining T cells.

### T Cell Modification Comprising an Altered Expression or Activity of GATA3 or FOXO1

The various aspects of the invention as disclosed in this specification are based, at least in part, on the novel discovery of useful markers, marker signatures and molecular targets associated with immune cell dysfunction and/or activation. More particularly, certain of the present markers, marker signatures and molecular targets correlate with the loss of effector function of the immune cells and are advantageously distinct, separate or uncoupled from, or independent of the immune cell activation status. Certain other of the present markers, marker signatures and molecular targets correlate with immune cell activation and are advantageously distinct, separate or uncoupled from, or independent of the immune cell dysfunction status.

Previously, obtaining molecular signatures for T cell dysfunction has been complicated by the fact that T cell dysfunction arises from chronic T cell activation, whereby molecular signatures of T cell dysfunction and activation are closely intertwined. Hence, co-inhibitory receptors that mark dysfunctional T cells are also up-regulated during T cell activation, where they function to contract the effector T cell population and restore immune homeostasis. Furthermore, dysfunctional CD8⁺ T cells and activated CDS T cells both upregulate genes that regulate activation of the cell cycle, T cell homing and migration and effector molecules such as granzymes, and both down-regulate memory cell gene signatures (Wherry et al. 2007, supra; Doering et al. 2012, supra). Indeed, T cell “dysfunction” may have likely evolved as a physiological process to carefully balance T cell activation and self-regulation in the face of chronic antigen persistence, thereby limiting immunopathology and minimizing collateral damage to the host.

An integrated experimental and computational approach was used to systematically dissect the molecular pathways associated with activation and “dysfunction” within CDS tumor-infiltrating lymphocytes (TILs), allowing to uncouple molecular signatures for T cell dysfunction and activation. The present analysis identifies gene modules that are uniquely associated with the dysfunctional T cell state and activated T cell state, and key molecular nodes that control them. The present markers, marker signatures and molecular targets thus provide for new ways to evaluate and modulate immune responses, such as to specifically evaluate and target the dysfunctional T cell state while leaving T cell activation programs intact.

Accordingly, an aspect of the invention provides an isolated immune cell modified to comprise an altered expression or activity of GATA3 or FOXO1. Further aspects provide an isolated immune cell modified to comprise an altered expression or activity of: i) one or more genes or gene products selected from the group consisting of the genes or gene products listed in Table 3 of US Pat. App. Pub. 2019/0100801, part “Dysfunction module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation module”: iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naive/Memory like module”. A further aspect provides a cell population of said modified immune cells.

Another aspect relates to a method for generating said modified immune cell, the method comprising (i) providing an isolated immune cell, and (ii) modifying said isolated immune cell such as to comprise an altered expression or activity of GATA3 or FOXO1. Further aspects provide a method for generating said modified immune cell, the method comprising (i) providing an isolated immune cell, and (ii) modifying said isolated immune cell such as to comprise an altered expression or activity of: i) one or more genes or gene products selected from the group consisting of the genes or gene products listed in Table 3 of US Pat. App. Pub. 2019/0100801, part “Dysfunction module”, Table 5A or Table 5B of US Pat. App. Pub. 2019/0100801, ii) one or more genes or gene products selected from the group consisting of the genes or gene products listed in Table 3 of US Pat. App. Pub. 2019/0100801, part “Activation module”: iii) one or more genes or gene products selected from the group consisting of the genes or gene products listed in Table 3 of US Pat. App. Pub. 2019/0100801, part “Dysfunction/Activation Module”, and/or iv) one or more genes or gene products selected from the group consisting of the genes or gene products listed in Table 3 of US Pat. App. Pub. 2019/0100801, part “Naive/Memory_like_module”.

A further aspect of the invention provides an isolated immune cell modified to comprise an agent capable of inducibly altering expression or activity of GATA3 or FOXO 1. Further aspects provide an isolated immune cell modified to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Nai've/Memory_like_module”. A further aspect provides a cell population of said modified immune cells.

Another aspect relates to a method for generating said modified immune cell, the method comprising (i) providing an isolated immune cell, and (ii) modifying said isolated immune cell such as to comprise an agent capable of inducibly altering expression or activity of GAT A3 or FOXO1. Further aspects provide a method for generating said modified immune cell, the method comprising (i) providing an isolated immune cell, and (ii) modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naive/Memory_like_module”.

Another aspect of the invention provides a pharmaceutical composition comprising the isolated immune cell or the cell population as defined above.

A further aspect of the invention relates to the isolated immune cell or the cell population as defined above for use in therapy,

Another aspect of the invention provides the isolated immune cell or the cell population as defined above for use in immunotherapy or adoptive immunotherapy.

A further aspect of the invention relates to a method of treating a subject in need thereof comprising administering to said subject the isolated immune cell or the cell population as defined above.

Another aspect of the invention provides a method of treating a subject in need thereof, comprising: (a) providing an isolated immune cell from the subject, or isolating an immune cell from a subject; (b) modifying said isolated immune cell such as to comprise an altered expression or activity of GATA3 and/or FOXO1, or modifying said isolated immune cell such as to comprise an agent capable of inducibly altering expression or activity of GATA3 and/or FOXO1; and (c) reintroducing the modified isolated immune cell to the subject. Further aspects provide a method of treating a subject in need thereof, comprising: (a) providing an isolated immune cell from the subject, or isolating an immune cell from a subject; (b) modifying said isolated immune cell such as to comprise an altered expression or activity of, or modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction__module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naive/Memory_like module”; and (c) reintroducing the modified isolated immune cell to the subject.

The method of treatment may be for a condition, disease or disorder where an enhanced immune response is required, such as but not limited to a cancer, or a condition, disease or disorder where a decreased immune response is required, such as but not limited to an autoimmune disease. The immune cell may be modified, such that expression of a gene signature is altered. The immune cell may be modified by treatment with an agent specific for downregulating expression or activity of at least one gene of one gene signature. The immune cell may be modified by treatment with an agent specific for upregulating expression or activity in at least one gene of an opposing gene signature. A gene in the dysfunctional gene signature and a gene in the activation signature may be such modified. Not being bound by a theory, cancer may be treated by obtaining a dysfunctional T cell and treating with an agent that activates the cell. Not being bound by a theory, introducing dysfunctional cells to a subject with an autoimmune disease may be performed. Dysfunctional cells secrete suppressive cytokines that may suppress immune cells causing the autoimmunity. A gene, gene signature or immune cell may be modified ex vivo. A gene, gene signature or immune cell may be modified ex vivo. A gene, gene signature or immune cell may be modified in vivo. Not being bound by a theory, modifying immune cells in vivo, such that dysfunctional immune cells are decreased can provide a therapeutic effect by enhancing an immune response in a subject. A gene, gene signature or immune cell may be modified by a small molecule, a DNA targeting agent, or a therapeutic antibody or antibody fragment thereof. As described herein, a DNA targeting agent may be a CRISPR system.

In another aspect, a method of treatment may comprise treating a subject with an agent specific for, e.g., capable of suppressing or activating, a cell type as defined by any one gene signature as taught herein, e.g., any one of the gene signatures, or portions thereof, as set forth in Table 3 of US Pat. App. Pub. 2019/0100801, Table 5A or Table 5B of US Pat. App. Pub. 2019/0100801. In certain embodiments, the agent is capable of suppressing an immune cell defined by any one of the gene signatures set forth in Table 3 of US Pat. App. Pub. 2019/0100801, Table 5A or Table 5B of US Pat. App. Pub. 2019/0100801. In certain other embodiments, the agent is capable of activating an immune cell defined by any one of the gene signatures set forth in Table 3 of US Pat. App. Pub. 2019/0100801, Table 5A or Table 5B of US Pat. App. Pub. 2019/0100801. In a preferred embodiment a dysfunctional T cell is targeted with an agent specific for a gene present only in the dysfunctional gene signature. In another embodiment an activated T cell is targeted with an agent specific for a gene present only in the activation gene signature. The gene may encode a surface protein. The agent may be a drug conjugated antibody. Not being bound by a theory, suppressing, such as by ablating dysfunctional T cells can increase cellular mediated toxicity of remaining T cells.

A further aspect of the invention relates a method of detecting dysfunctional immune cells comprising detection of a gene expression signature comprising one or more markers of dysfunction selected from the group consisting of GATA3, FOXO1, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14.

A related aspect relates to a method of detecting dysfunctional immune cells comprising detection of a gene expression signature comprising one or more markers of dysfunction selected from the group consisting of GATA3, FOXO1, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14. A further aspect of the invention relates to a method of detecting dysfunctional immune cells comprising detection of a gene expression signature comprising one or more markers of dysfunction selected from the group consisting of NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, POU2AF1, GATA3, B3GNT2, FAS, PIAS2, FOXO1, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, and BTLA. A related aspect provides a method of detecting dysfunctional immune cells comprising detection of a gene expression signature comprising one or more markers of dysfunction selected from the group consisting of the markers listed in Table 3 of US Pat. App. Pub. 2019/0100801, part “Dysfunction module”, Table 5A or Table 5B of US Pat. App. Pub. 2019/0100801.

A further aspect of the invention relates to a method of detecting activated immune cells comprising detection of a gene expression signature comprising one or more markers of activation selected from the group consisting of TMCO1, PRMT5, EXOC4, TYR, HDHD2, RCN1, LMNB2, TCTEX1D2, VMA21, HCFC2, MRPS27, DUSP19, CD200R4, SRSF10, NAP1L4, ZADH2, ERGIC1, STARD3NL, RCC1, CD38, ZFP142, METTL10, MOGS, S100PBP, AREG, 1700052N19RIK, NDUFA13, RFT1, TAF12, ELP2, TONSL, FANCG, PIGF, GNG2, HIST1H1E, MINA, NDUFAB1, AP1M1, DYNLT1C, JAGN1, CERS4, METTL3, GCDH, RBX1, HAUS4, TFIP11, BC026590, PSMB9, PTPN23, PIAS3, TMEM129, DPYSL2, TMEM209, CALU, EXOSC1, PQLC3, ACO1, PDIA4, POLR3K, NTAN1, PSMB3, ARFIP1, PHF11B, MYEF2, TIMM50, ACAD8, RDM1, CCNH, TMEM41A, PLAA, MEAF6, EXOSC3, QRSL1, UPF1, ANXA6, FTSJD2, PRPSAP1, ARSB, GM11127, HNRNPA2B1, NUP35, RPRD1B, NCBP2, HIST1H3E, KIFC1, MLH1, CD200R1, CPSF6, CDT1, PPM1G, MRPS33, PRADC1, GBP3, RAD17, MTHFSD, FOXRED1, TAX1BP3, C1D, TPM3, D16ERTD472E, SARS2, 0610009O20RIK, ARPP19, ASRGL1, SDF2L1, TBCC, MYG1, SEPHS1, DYNC1LI1, ZBTB38, TARDBP, SLC9A8, TYK2, THUMPD3, MRPL16, ACOT8, LRRK1, HMGB1, HSPA1B, TCEA1, MAVS, POFUT2, VPS53, RIT1, SNAPC1, DNAAF2, COMMD10, PMPCB, EHBP1L1, ADAT3, DOHH, LSM4, PTCD1, GMPPB, LAMTOR1, DRG2, CDCA7L, SSBP1, ANAPC15, NAGLU, AKR1B3, PAOX, EIF4E2, GPAA1, RAD50, STX18, GRPEL1, VMP1, REXO2, HIST1H1C, ZFP429, GGH, TAF6, COMMD3, PARL, RBM18, 2700029M09RIK, EXOSC4, ABHD10, DNAJC14, DPCD, ATPBD4, SERPINA3F, CTCF, LMAN1, NEU3, EIF2D, HAUS5, USF1, AAR2, FARSB, COG4, COG2, FKBP2, SLC35A1, DPY30, ALDH3A2, 1110008P14RIK, KLRE1, ZDHHC6, RAD18, TSPAN4, METTL20, NUDT16L1, TMEM167, IPP, INIP, REEP4, ERP44, GIMAP7, CYB5B, ACAT2, ANAPC5, PEX19, PUF60, SLBP, MTG1, ACTR10, CCDC127 and KPNB1.

A further aspect of the invention relates to a method of detecting dysfunctional and/or activated immune cells comprising detection of a gene expression signature comprising one or more markers selected from the group consisting of SEC23A, ACTN4, MTMR1, TIGIT, TRIP13, NCOR2, CCDC50, LPCAT1, GMNN, CCR8, FLNA, CIAPIN1, TK1, E430025E21RIK, ENDOD1, RGS8, SLC35A3, ARL6IP1, CALM3, MCM3, MKI67, SLC25A13, SUOX, AP3S1, NAA38, NUCKS1, CDCA8, UHRF2, RAD54L, PSAT1, FEM1B, MCM5, CCNB2, CX3CR1, SH3BGRL, HIST1H1B, CASP3, DNMT3A, CCNA2, DUT, STMN1, MEMO1, WHSC1, BUB1B, FKBP1A, CCT7, ATP6V1A, POLA1, GTDC1, RPPH1, NR4A2, AP2M1, FUT7, CDCA3, STRN, CHAF1A, IL18RAP, ST14, ADAMTS14, ACTG1, KIF13B, PTPN5, RAB8B, SERPINE2, CSTF2, EIF4H, GM5069, TMEM48, CTLA4, GM9855, EZH2, MMS22L, RAD51, TPX2, METRN, TMEM126A, HIF1A, MSH6, NCAPD2, UHRF1, ALCAM, HMGN2, MAP4, POLD1, DGKZ, LCP1, AURKB, MRPS22, 2810417H13RIK, WDR76, GALNT3, IPO5, GM5177, NAB2, CISH, ARF5, CENPH, STAP1, KIF15, HIST1H2AG, CDC45, PTPN11, GINS1, TFDP1, MLF2, PGP, POLE, HIST1H2AO, IL10RA, LDHA, SERPINB6A, ASNSD1, LCLAT1, CALR, LGALS1, NDFIP2, GPD2, RRM1, TPI1, DUSP14, MAD2L1, MLEC, CRMP1, DTL, PDCD1, INTS7, WDR3, MED14, EEA1, UAP1, FAR1, GAPDH, YWHAH, MMD, CSF1, HN1L, MDFIC, DUSP4, IL2RA, ALDOA, HIST2H3B, ENO1, SIVA1, TNFRSF4, TNFRSF9, CSRP1, IGFBP7, MCM6, RDX, KIF2C, RBL2, BCL2A1B, HIST1H3C, ATP5B, CIT, B4GALT5, HELLS, TRPS1, FAM129A, TXN1, HSP90AB1, H2AFZ, METAP2, DESI1, FIGNL1, LIN54, CAPG, SYNE3, AI836003, LIG1, HCFC1, GARS, SMARCA5, PGK1, PPP2R4, BCL2A1D, PPP1CA, RBPJ, BHLHE40, SLC16A3, DNMT1, S100A4, PKM, PRELID1, KIF20A, ITGAV, TWSG1, TACC3, ATP5F1, RQCD1, ANKRD52, RGS16, ANXA2, TMPO, ATP10A, PRIM1, ZFP207, STX11, RPS2, and TOPBP1.

A further aspect of the invention relates to a method of detecting naive-memory-like immune cells comprising detection of a gene expression signature comprising one or more markers selected from the group consisting of GPR183, THA1, TREML2, ZNRF3, CDK2AP2, CREB3, RPS16, BLOC1S2A, ATP1B3, BLNK, RPS29, SHARPIN, TSC22D1, KLRA1, HSD11B1, RPS15, AKAP8L, PHC1, RPL31, S1PR1, GM5547, SRSF5, ACSS2, ADK, AMICA1, ATP1B1, CNP, SNHG8, FCRLA, H2-T23, RAB33B, TLR12, RPF1, SP140, SH3GL1, CTSL, RPGRIP1, 5430417L22RIK, CXXC5, RABGGTA, KCNJ8, DYM, FRAT1, SPIB, ADRB2, COX6A2, TMEM219, GPR18, CCPG1, PLCB2, CALM2, KYNU, CRLF3, IDNK, TNFRSF26, DNAJB9, TXNIP, UPB1, GM11346, PHF1, RPL18A, DNTT, HAAO, PIM2, RABAC1, APOPTI, BIN2, OXR1, GPR171, RASGRP2, SLC9A9, 5830411N06RIK, PIAS1, PYDC3, ZCCHC18, TCSTV3, KLRA7, NPC2, CD180, SMIM14, P2RY14, PDLIM1, MYLIP, PDE2A, PPIF, KLRA17, FBXO32, DIRC2, ELOVL6, PJA1, SP110, KLRA6, USP7, HCST, KLRA23, GAB3, TOM1, ACP5, PBLD1, SMPD5, EVI2A, KLF13, MFSD11, IFNGR1, POU6F1, USEl, HDAC4, SMIM5, MAF1, 1810034E14RIK, TSC22D3, GAS5, RPL21, RELL1, SERTAD2, BC147527, KMO, SKAP1, TCF4, SP100, RNF167, TMEM59, IRGM1, CD69, DNAJC7, PIK3IP1, TAZ, HAVCR1, LY6D, RPL23, DAPP1, FLT3, ITM2B, NUCB2, RPS14, GIMAP9, HBP1, MAN2A2, RNF122, SOCS3, CD7, PNCK, 2610019F03RIK, SLC27A1, BPTF, H2-Q9, KLHL6, RPL17, SEMA4B, LDLRAD4, TCEA2, GM14207, CIRBP, FAM189B, ZFP707, ATP10D, RNASET2A, ATP2A1, BST2, EYA2, IRF7, ITPR2, STK17B, CYBASC3, TRIM11, KLK1B27, ZMYND8, LEF1, RNASE6, EIF4A2, HS3ST1, NIPBL, STX4A, UGCG, CAMKlD, PPFIA4, UVRAG, CDKN2D, ZBTB21, LEFTY1, APBB1IP, GIMAP3, H13, RGS10, RNF138, RPL12, SLC7A6OS, FADS2, SELPLG, CXCR4, GPR146, ZFP386, BCL11A, TRIM34A, RPS7, TLR9, PACSIN1, PAIP1, PGAM2 and JAKMIP1.

A yet further aspect of the invention relates to a kit of parts comprising means for detection of the above signature of dysfunction. Also provided is a kit of parts comprising means for detection of the signature of dysfunction, activation, activation and/or dysfunction, or memory as taught herein.

Another aspect of the invention provides a method for determining whether or not an immune cell has a dysfunctional immune phenotype and/or whether or not an immune cell would benefit from upregulation of an immune response, said method comprising: (a) determining in said immune cell the expression of GATA3 and/or FOXO1, whereby expression of GAT A3 and/or FOXO 1 indicates that the immune cell has a dysfunctional immune phenotype and/or would benefit from upregulation of an immune response; or (b) determining in said immune cell the expression of the signature of dysfunction as defined herein, whereby expression of the signature indicates that the immune cell has a dysfunctional immune phenotype and/or would benefit from upregulation of an immune response.

Also provided is a method for determining whether or not an immune cell has an activation, activation and/or dysfunction or memory immune phenotype and/or whether or not an immune cell would benefit from modulation (e.g., downregulation or upregulation) of an immune response, said method comprising: determining in said immune cell the expression of the signature of activation, activation and/or dysfunction, or memory, as defined herein, whereby expression of the signature indicates that the immune cell has respectively an activation, activation and/or dysfunction or memory immune phenotype and/or would benefit from modulation of an immune response.

A further aspect of the invention provides a method for determining whether or not a patient would benefit from a therapy aimed at reducing dysfunction of immune cells or a therapy-aimed at upregulating of an immune response, the method comprising: (a) determining, in immune cells from said patient the expression of GATA3 and/or FOXO1, whereby expression of GATA3 and/or FOXO 1 indicates that the patient will benefit from the therapy, or (b) determining, in immune cells from said patient the expression of the signature of dysfunction as defined above, whereby expression of the signature indicates the patient will benefit from the therapy,

Also provided is a method for determining whether or not a patient would benefit from a therapy aimed at modulating (e.g., reducing or increasing) activation, activation and/or dysfunction or memory phenotype of immune cells, or a therapy aimed at modulating (e.g., reducing or increasing) of an immune response, said method comprising determining, in immune cells from said patient the expression of the signature of activation, activation and/or dysfunction, or memory, as defined herein, whereby expression of the signature indicates that the patient will benefit from the therapy aimed at modulating respectively the activation, activation and/or dysfunction or memory phenotype of immune cells, or will benefit from the therapy aimed at modulating the immune response.

Another aspect of the invention relates to a method for determining the efficacy of a treatment of a patient with a therapy, said method comprising: (a) determining in immune cells from said patient the expression of GATA3 and/or FOXO1 before and after said treatment and determining the efficacy of said therapy based thereon, whereby unchanged or increased expression of GATA3 and/or FOXO1 indicates that the treatment should be adjusted: or (b) determining in immune cells from said patient the expression of the signature of dysfunction as defined above before and after said treatment and determining the efficacy of said therapy based thereon, whereby unchanged or increased expression of the signature indicates that the treatment should be adjusted.

Also provided is a method for determining the efficacy of a treatment of a patient with a therapy, said method comprising determining in immune cells from said patient the expression of the signature of activation, activation and/or dysfunction, or memory, as defined herein, before and after said treatment and determining the efficacy of said therapy based thereon, whereby unchanged or increased expression of the signature indicates that the treatment should be adjusted.

Another aspect of the invention provides a method for determining the suitability of a compound as a checkpoint inhibitor, said method comprising: (a) contacting an immune cell expressing GATA3 and/or FOXO1 with said compound and determining whether or not said compound can affect the expression of GATA3 and/or FOXO1 by said cell, whereby decreased expression indicates that the compound is suitable as a checkpoint inhibitor, or (b) contacting an immune cell expressing the signature of dysfunction as defined above with said compound and determining whether or not said compound can affect the expression of the signature by said cell, whereby decreased expression indicates that the compound is suitable as a checkpoint inhibitor. [0042] Also provided is a method for determining the suitability of a compound as a checkpoint inhibitor, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable as a checkpoint inhibitor (e.g., whereby increased expression of the signature of activation indicates that the compound is suitable as a checkpoint inhibitor).

A further aspect of the invention provides a method for determining the suitability of a compound for reducing an dysfunctional immune phenotype and/or upregulating of an immune response, said method comprising: (a) contacting an immune cell expressing GATA3 and/or FOXO1 with said compound and determining whether or not said compound can affect the expression of GATA3 and/or FOXO1 by said cell, whereby decreased expression indicates that the compound is suitable for reducing dysfunctional immune phenotype and/or upregulating of an immune response; or (b) contacting an immune cell expressing the signature of dysfunction as defined above with said compound and determining whether or not said compound can affect the expression of the signature by said cell, whereby decreased expression indicates that the compound is suitable for reducing dysfunctional immune phenotype and/or upregulating of an immune response.

Also provided is a method for determining the suitability of a compound for modulating (e.g., reducing or increasing) activation, activation and/or dysfunction or memory phenotype of immune cells, and/or modulating (e.g., reducing or increasing) of an immune response, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable for modulating respectively the activation, activation and/or dysfunction or memory phenotype of immune cells, and/or modulating of the immune response.

A yet another aspect of the invention provides a method for stratification of immune cells into one or more cell populations comprising at least a first cell population having a comparatively more dysfunctional immune phenotype and a second population having a comparatively less dysfunctional immune phenotype, comprising: (a) determining in said immune cells the expression of GATA3 and/or FOXO1, and allotting cells having no or comparatively lower expression of GATA3 and/or FOXO1 into said second population, and cells having comparatively higher expression of GATA3 and/or FOXO1 into said first population; or (b) determining in said immune cells the expression of the signature of dysfunction as defined above, and allotting cells having no or comparatively lower expression of said signature into said second population, and cells having comparatively higher expression of said signature into said first population.

Also provided is a method for stratification of immune cells into one or more cell populations comprising at least a first cell population having a comparatively more activation, activation and/or dysfunction or memory phenotype and a second population having a comparatively less activation, activation and/or dysfunction or memory phenotype, said method comprising determining in said immune cells the expression of the signature of activation, activation and/or dysfunction, or memory, as defined herein, and allotting cells having no or comparatively lower expression of said signature into said second population, and cells having comparatively higher expression of said signature into said first population.

Also provided is a method for stratification of immune cells into one or more cell populations comprising at least a first cell population having a comparatively more activation, activation and/or dysfunction or memory phenotype and a second population having a comparatively less activation, activation and/or dysfunction or memory phenotype, said method comprising determining in said immune cells the expression of the signature of activation, activation and/or dysfunction, or memory, as defined herein, and allotting cells having no or comparatively lower expression of said signature into said second population, and cells having comparatively higher expression of said signature into said first population.

A yet another aspect provides a method of isolating an immune cell as taught herein comprising binding of an affinity ligand to a signature gene expressed on the surface of the immune cell.

A further aspect provides a method of treating a subject in need thereof, comprising administering to said subject an agent capable of modulating the immune cell as taught herein.

A further aspect provides a method of treatment comprising administering one or more checkpoint inhibitors to a patient in need thereof, wherein immune cells obtained from the patient have a gene signature as taught herein, such as the gene signature of dysfunction as taught herein.

Engineered T cells having altered expression or activity of POU2AF1

The various aspects of the invention as disclosed in this specification are based, at least in part, on the novel discovery of useful markers, marker signatures and molecular targets associated with immune cell dysfunction and/or activation. More particularly, certain of the present markers, marker signatures and molecular targets correlate with the loss of effector function of the immune cells and are advantageously distinct, separate or uncoupled from, or independent of the immune cell activation status. Certain other of the present markers, marker signatures and molecular targets correlate with immune cell activation and are advantageously distinct, separate or uncoupled from, or independent of the immune cell dysfunction status.

Previously, obtaining molecular signatures for T cell dysfunction has been complicated by the fact that T cell dysfunction arises from chronic T cell activation, whereby molecular signatures of T cell dysfunction and activation are closely intertwined. Hence, co-inhibitory receptors that mark dysfunctional T cells are also up-regulated during T cell activation, where they function to contract the effector T cell population and restore immune homeostasis. Furthermore, dysfunctional CD8⁺ T cells and activated CD8⁺ T cells both upregulate genes that regulate activation of the cell cycle, T cell homing and migration and effector molecules such as granzymes, and both down-regulate memory cell gene signatures (Wherry et al. 2007, supra; Doering et al. 2012, supra). Indeed, T cell “dysfunction” may have likely evolved as a physiological process to carefully balance T cell activation and self-regulation in the face of chronic antigen persistence, thereby limiting immunopathology and minimizing collateral damage to the host.

An integrated experimental and computational approach was used to systematically dissect the molecular pathways associated with activation and “dysfunction” within CD8⁺ tumor-infiltrating lymphocytes (TILs), allowing to uncouple molecular signatures for T cell dysfunction and activation. The present analysis identifies gene modules that are uniquely associated with the dysfunctional T cell state and activated T cell state, and key molecular nodes that control them. The present markers, marker signatures and molecular targets thus provide for new ways to evaluate and modulate immune responses, such as to specifically evaluate and target the dysfunctional T cell state while leaving T cell activation programs intact.

Accordingly, an aspect of the invention provides an isolated immune cell modified to comprise an altered expression or activity of POU2AF1. Further aspects provide an isolated immune cell modified to comprise an altered expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”. A further aspect provides a cell population of said modified immune cells.

Another aspect relates to a method for generating said modified immune cell, the method comprising (i) providing an isolated immune cell, and (ii) modifying said isolated immune cell such as to comprise an altered expression or activity of POU2AF1. Further aspects provide a method for generating said modified immune cell, the method comprising (i) providing an isolated immune cell, and (ii) modifying said isolated immune cell such as to comprise an altered expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”.

A further aspect of the invention provides an isolated immune cell modified to comprise an agent capable of inducibly altering expression or activity of POU2AF1. Further aspects provide an isolated immune cell modified to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”. A further aspect provides a cell population of said modified immune cells.

Another aspect relates to a method for generating said modified immune cell, the method comprising (i) providing an isolated immune cell, and (ii) modifying said isolated immune cell such as to comprise an agent capable of inducibly altering expression or activity of POU2AF1. Further aspects provide a method for generating said modified immune cell, the method comprising (i) providing an isolated immune cell, and (ii) modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”.

Another aspect of the invention provides a pharmaceutical composition comprising the isolated immune cell or the cell population as defined above.

A further aspect of the invention relates to the isolated immune cell or the cell population as defined above for use in therapy.

Another aspect of the invention provides the isolated immune cell or the cell population as defined above for use in immunotherapy or adoptive immunotherapy.

A further aspect of the invention relates to a method of treating a subject in need thereof, comprising administering to said subject the isolated immune cell or the cell population as defined above.

Another aspect of the invention provides a method of treating a subject in need thereof, comprising: (a) providing an isolated immune cell from the subject, or isolating an immune cell from a subject; (b) modifying said isolated immune cell such as to comprise an altered expression or activity of POU2AF1, or modifying said isolated immune cell such as to comprise an agent capable of inducibly altering expression or activity of POU2AF1; and (c) reintroducing the modified isolated immune cell to the subject. Further aspects provide a method of treating a subject in need thereof, comprising: (a) providing an isolated immune cell from the subject, or isolating an immune cell from a subject; (b) modifying said isolated immune cell such as to comprise an altered expression or activity of, or modifying said isolated immune cell such as to comprise an agent capable of inducibly altering expression or activity of: i) one or more genes or gene products selected from the group consisting of the genes or gene products listed in Table 3 of US Pat. App. Pub. 2019/0100801, part “Dysfunction_module”, Table 5A or Table 5B of US Pat. App. Pub. 2019/0100801; ii) one or more genes or gene products selected from the group consisting of the genes or gene products listed in Table 3 of US Pat. App. Pub. 2019/0100801, part “Activation_module”; iii) one or more genes or gene products selected from the group consisting of the genes or gene products listed in Table 3 of US Pat. App. Pub. 2019/0100801, part “Dysfunction/Activation Module”; and/or iv) one or more genes or gene products selected from the group consisting of the genes or gene products listed in Table 3 of US Pat. App. Pub. 2019/0100801, part “Naïve/Memory_like_module”; and (c) reintroducing the modified isolated immune cell to the subject.

The method of treatment may be for a condition, disease or disorder where an enhanced immune response is required, such as but not limited to a cancer, or a condition, disease or disorder where a decreased immune response is required, such as but not limited to an autoimmune disease. The immune cell may be modified, such that expression of a gene signature is altered. The immune cell may be modified by treatment with an agent specific for downregulating expression or activity of at least one gene of one gene signature. The immune cell may be modified by treatment with an agent specific for upregulating expression or activity in at least one gene of an opposing gene signature. A gene in the dysfunctional gene signature and a gene in the activation signature may be such modified. Not being bound by a theory, cancer may be treated by obtaining a dysfunctional T cell and treating with an agent that activates the cell. Not being bound by a theory, introducing dysfunctional cells to a subject with an autoimmune disease may be performed. Dysfunctional cells secrete suppressive cytokines that may suppress immune cells causing the autoimmunity. A gene, gene signature or immune cell may be modified ex vivo. A gene, gene signature or immune cell may be modified ex vivo. A gene, gene signature or immune cell may be modified in vivo. Not being bound by a theory, modifying immune cells in vivo, such that dysfunctional immune cells are decreased can provide a therapeutic effect by enhancing an immune response in a subject. A gene, gene signature or immune cell may be modified by a small molecule, a DNA targeting agent, or a therapeutic antibody or antibody fragment thereof. As described herein, a DNA targeting agent may be a CRISPR system.

In another aspect, a method of treatment may comprise treating a subject with an agent specific for, e.g., capable of suppressing or activating, a cell type as defined by any one gene signature as taught herein, e.g., any one of the gene signatures, or portions thereof, as set forth in Table 3 of US Pat. App. Pub. 2019/0100801, Table 5A or Table 5B of US Pat. App. Pub. 2019/0100801. In certain embodiments, the agent is capable of suppressing an immune cell defined by any one of the gene signatures set forth in Table 3 of US Pat. App. Pub. 2019/0100801, Table 5A or Table 5B of US Pat. App. Pub. 2019/0100801. In certain other embodiments, the agent is capable of activating an immune cell defined by any one of the gene signatures set forth in Table 3 of US Pat. App. Pub. 2019/0100801, Table 5A or Table 5B of US Pat. App. Pub. 2019/0100801. In a preferred embodiment, a dysfunctional T cell is targeted with an agent specific for a gene present only in the dysfunctional gene signature. In another embodiment an activated T cell is targeted with an agent specific for a gene present only in the activation gene signature. The gene may encode a surface protein. The agent may be a drug conjugated antibody. Not being bound by a theory, suppressing, such as by ablating dysfunctional T cells can increase cellular mediated toxicity of remaining T cells.

A further aspect of the invention relates to a method of detecting dysfunctional immune cells comprising detection of a gene expression signature comprising one or more markers of dysfunction selected from the group consisting of GATA3, FOXO1, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14.

A related aspect relates to a method of detecting dysfunctional immune cells comprising detection of a gene expression signature comprising one or more markers of dysfunction selected from the group consisting of GATA3, FOXO1, POU2AF1, BTLA, NRP1, NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, B3GNT2, FAS, PIAS2, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, MT1, MT2, PD1, CTLA4, TIGIT, TIM3, LAG3, KLRC1, CD160, CD274, IDO, CD200, CD244, KLRD1, LAIR1, CEACAM1, KLRA7, TNFRSF9, TNFRSF4, TNFSF4, TNFRSF18, TNFSF11, CD27, CD28, CD86, ICOS, and TNFSF14. A further aspect of the invention relates to a method of detecting dysfunctional immune cells comprising detection of a gene expression signature comprising one or more markers of dysfunction selected from the group consisting of NPEPPS, NOTCH2, CABLES1, CERK, MTMR3, RELB, KLF3, CAMK2D, CCNG2, SLC25A33, PIM3, RNF149, SWAP70, PINK1, RAB2A, FAM168B, MAP2K7, MIR466I, ASAP1, GRASP, POU2AF1, GATA3, B3GNT2, FAS, PIAS2, FOXO1, SEC24B, TUBB2B, PARP3, PIGH, BRAP, ATP6V0D1, IFT80, FRRS1, GPR132, SFPI1, SH2B3, WFDC17, CD74, TBC1D22B, PHC2, TRAT1, SLAMF6, YPEL3, RARA, GM9159, MAN1A, CRTC3, MKRN1, BCL6, CLN6, MYB, NDUFV1, SLC28A2, FBXL20, SCIN, LGMN, WTAP, BCL3, SLC2A6, IL2RG, SNTB1, KDM5B, UTP15, LATS2, RASSF2, IFI30, KDM4B, IER5, CD5, MNDAL, PCGF5, GPR35, SPRY1, TNIP1, CSNK1D, NSMCE1, NR4A1, OSBPL11, PNRC1, ITGAE, SNX18, TMEM55B, IKZF2, ISCU, FAM196B, TMEM243, ZFP62, RASGEF1B, DTWD1, GNA13, JAK2, EIF3F, CCR7, SGPP1, SLAMF7, QRICH1, EML4, CACNB3, ATG7, SUV420H1, HBS1L, RAB2B, H2-AB1, DGKD, SESN3, ELK4, PIM1, JOSD1, SPIN1, LILRB3, CHIC2, H2-DMB2, TPRGL, IL4I1, ACAP2, SUDS3, ABCA3, TNRC6A, RPS5, MPLKIP, NEK7, SOD1, CRY1, MIDN, RBMS1, PRAMEF8, ATP2A3, RPS6KB2, MRS2, PLEKHG2, TCF12, MED8, LIMD1, SMIM8, KDM3A, BACH2, ILVBL, 4930523C07RIK, CD28, SLC52A2, ACBD6, ANKIB1, BANK1, KLHDC2, AHR, MLXIP, TRAF4, MFSD6, GM4070, PFKFB3, ANTXR2, GRWD1, MAP1LC3A, HP, RAP2B, TRPC4AP, SMG1, DEDD, UNC13D, RAB6A, CCDC88B, TNFRSF13C, TRP53INP1, SFPQ, CD44, HDAC8, UBE2D3, EIF3I, P2RY6, TBC1D4, 0610012G03RIK, RASSF5, AHCYL2, NDUFS4, PTP4A3, RNF111, SMAP1, IFITM3, PPAPDC1B, PRMT2, RPLP0, FOXN3, IFITM6, IFT20, CTAGE5, ZFP622, PPP2CA, WDR82, POLB, BRD4, UBL3, SLC12A9, NCOA7, TRAPPC3, MEF2D, LACTB, MALT1, LYZ2, CD160, CD274, PTGER4, and BTLA. A related aspect provides a method of detecting dysfunctional immune cells comprising detection of a gene expression signature comprising one or more markers of dysfunction selected from the group consisting of the markers listed in Table 3 of US Pat. App. Pub. 2019/0100801, part “Dysfunction_module”, Table 5A or Table 5B of US Pat. App. Pub. 2019/0100801.

A further aspect of the invention relates to a method of detecting activated immune cells comprising detection of a gene expression signature comprising one or more markers of activation selected from the group consisting of TMCO1, PRMT5, EXOC4, TYR, HDHD2, RCN1, LMNB2, TCTEX1D2, VMA21, HCFC2, MRPS27, DUSP19, CD200R4, SRSF10, NAP1L4, ZADH2, ERGIC1, STARD3NL, RCC1, CD38, ZFP142, METTL10, MOGS, S100PBP, AREG, 1700052N19RIK, NDUFA13, RFT1, TAF12, ELP2, TONSL, FANCG, PIGF, GNG2, HIST1H1E, MINA, NDUFAB1, AP1M1, DYNLT1C, JAGN1, CERS4, METTL3, GCDH, RBX1, HAUS4, TFIP11, BC026590, PSMB9, PTPN23, PIAS3, TMEM129, DPYSL2, TMEM209, CALU, EXOSC1, PQLC3, ACO1, PDIA4, POLR3K, NTAN1, PSMB3, ARFIP1, PHF11B, MYEF2, TIMM50, ACAD8, RDM1, CCNH, TMEM41A, PLAA, MEAF6, EXOSC3, QRSL1, UPF1, ANXA6, FTSJD2, PRPSAP1, ARSB, GM11127, HNRNPA2B1, NUP35, RPRD1B, NCBP2, HIST1H3E, KIFC1, MLH1, CD200R1, CPSF6, CDT1, PPM1G, MRPS33, PRADC1, GBP3, RAD17, MTHFSD, FOXRED1, TAX1BP3, C1D, TPM3, D16ERTD472E, SARS2, 0610009O20RIK, ARPP19, ASRGL1, SDF2L1, TBCC, MYG1, SEPHS1, DYNC1LI1, ZBTB38, TARDBP, SLC9A8, TYK2, THUMPD3, MRPL16, ACOT8, LRRK1, HMGB1, HSPA1B, TCEA1, MAVS, POFUT2, VPS53, RIT1, SNAPC1, DNAAF2, COMMD10, PMPCB, EHBP1L1, ADAT3, DOHH, LSM4, PTCD1, GMPPB, LAMTOR1, DRG2, CDCA7L, SSBP1, ANAPC15, NAGLU, AKR1B3, PAOX, EIF4E2, GPAA1, RAD50, STX18, GRPEL1, VMP1, REXO2, HIST1H1C, ZFP429, GGH, TAF6, COMMD3, PARL, RBM18, 2700029M09RIK, EXOSC4, ABHD10, DNAJC14, DPCD, ATPBD4, SERPINA3F, CTCF, LMAN1, NEU3, EIF2D, HAUS5, USF1, AAR2, FARSB, COG4, COG2, FKBP2, SLC35A1, DPY30, ALDH3A2, 1110008P14RIK, KLRE1, ZDHHC6, RAD18, TSPAN4, METTL20, NUDT16L1, TMEM167, IPP, INIP, REEP4, ERP44, GIMAP7, CYB5B, ACAT2, ANAPC5, PEX19, PUF60, SLBP, MTG1, ACTR10, CCDC127 and KPNB1.

A further aspect of the invention relates to a method of detecting dysfunctional and/or activated immune cells comprising detection of a gene expression signature comprising one or more markers selected from the group consisting of SEC23A, ACTN4, MTMR1, TIGIT, TRIP13, NCOR2, CCDC50, LPCAT1, GMNN, CCR8, FLNA, CIAPIN1, TK1, E430025E21RIK, ENDOD1, RGS8, SLC35A3, ARL6IP1, CALM3, MCM3, MKI67, SLC25A13, SUOX, AP3S1, NAA38, NUCKS1, CDCA8, UHRF2, RAD54L, PSAT1, FEM1B, MCM5, CCNB2, CX3CR1, SH3BGRL, HIST1H1B, CASP3, DNMT3A, CCNA2, DUT, STMN1, MEMO1, WHSC1, BUB1B, FKBP1A, CCT7, ATP6V1A, POLA1, GTDC1, RPPH1, NR4A2, AP2M1, FUT7, CDCA3, STRN, CHAF1A, IL18RAP, ST14, ADAMTS14, ACTG1, KIF13B, PTPN5, RAB8B, SERPINE2, CSTF2, EIF4H, GM5069, TMEM48, CTLA4, GM9855, EZH2, MMS22L, RAD51, TPX2, METRN, TMEM126A, HIF1A, MSH6, NCAPD2, UHRF1, ALCAM, HMGN2, MAP4, POLD1, DGKZ, LCP1, AURKB, MRPS22, 2810417H13RIK, WDR76, GALNT3, IPO5, GM5177, NAB2, CISH, ARF5, CENPH, STAP1, KIF15, HIST1H2AG, CDC45, PTPN11, GINS1, TFDP1, MLF2, PGP, POLE, HIST1H2AO, IL10RA, LDHA, SERPINB6A, ASNSD1, LCLAT1, CALR, LGALS1, NDFIP2, GPD2, RRM1, TPI1, DUSP14, MAD2L1, MLEC, CRMP1, DTL, PDCD1, INTS7, WDR3, MED14, EEA1, UAP1, FAR1, GAPDH, YWHAH, MMD, CSF1, HN1L, MDFIC, DUSP4, IL2RA, ALDOA, HIST2H3B, ENO1, SIVA1, TNFRSF4, TNFRSF9, CSRP1, IGFBP7, MCM6, RDX, KIF2C, RBL2, BCL2A1B, HIST1H3C, ATP5B, CIT, B4GALT5, HELLS, TRPS1, FAM129A, TXN1, HSP90AB1, H2AFZ, METAP2, DESI1, FIGNL1, LIN54, CAPG, SYNE3, AI836003, LIG1, HCFC1, GARS, SMARCA5, PGK1, PPP2R4, BCL2A1D, PPP1CA, RBPJ, BHLHE40, SLC16A3, DNMT1, S100A4, PKM, PRELID1, KIF20A, ITGAV, TWSG1, TACC3, ATP5F1, RQCD1, ANKRD52, RGS16, ANXA2, TMPO, ATP10A, PRIM1, ZFP207, STX11, RPS2, and TOPBP1.

A further aspect of the invention relates to a method of detecting naïve-memory-like immune cells comprising detection of a gene expression signature comprising one or more markers selected from the group consisting of GPR183, THA1, TREML2, ZNRF3, CDK2AP2, CREB3, RPS16, BLOC1S2A, ATP1B3, BLNK, RPS29, SHARPIN, TSC22D1, KLRA1, HSD11B1, RPS15, AKAP8L, PHC1, RPL31, S1PR1, GM5547, SRSF5, ACSS2, ADK, AMICA1, ATP1B1, CNP, SNHG8, FCRLA, H2-T23, RAB33B, TLR12, RPF1, SP140, SH3GL1, CTSL, RPGRIP1, 5430417L22RIK, CXXC5, RABGGTA, KCNJ8, DYM, FRAT1, SPIB, ADRB2, COX6A2, TMEM219, GPR18, CCPG1, PLCB2, CALM2, KYNU, CRLF3, IDNK, TNFRSF26, DNAJB9, TXNIP, UPB1, GM11346, PHF1, RPL18A, DNTT, HAAO, PIM2, RABAC1, APOPTI, BIN2, OXR1, GPR171, RASGRP2, SLC9A9, 5830411N06RIK, PIAS1, PYDC3, ZCCHC18, TCSTV3, KLRA7, NPC2, CD180, SMIM14, P2RY14, PDLIM1, MYLIP, PDE2A, PPIF, KLRA17, FBXO32, DIRC2, ELOVL6, PJA1, SP110, KLRA6, USP7, HCST, KLRA23, GAB3, TOM1, ACP5, PBLD1, SMPD5, EVI2A, KLF13, MFSD11, IFNGR1, POU6F1, USEl, HDAC4, SMIM5, MAF1, 1810034E14RIK, TSC22D3, GAS5, RPL21, RELL1, SERTAD2, BC147527, KMO, SKAP1, TCF4, SP100, RNF167, TMEM59, IRGM1, CD69, DNAJC7, PIK3IP1, TAZ, HAVCR1, LY6D, RPL23, DAPP1, FLT3, ITM2B, NUCB2, RPS14, GIMAP9, HBP1, MAN2A2, RNF122, SOCS3, CD7, PNCK, 2610019F03RIK, SLC27A1, BPTF, H2-Q9, KLHL6, RPL17, SEMA4B, LDLRAD4, TCEA2, GM14207, CIRBP, FAM189B, ZFP707, ATP10D, RNASET2A, ATP2A1, BST2, EYA2, IRF7, ITPR2, STK17B, CYBASC3, TRIM11, KLK1B27, ZMYND8, LEF1, RNASE6, EIF4A2, HS3ST1, NIPBL, STX4A, UGCG, CAMKlD, PPFIA4, UVRAG, CDKN2D, ZBTB21, LEFTY1, APBB1IP, GIMAP3, H13, RGS10, RNF138, RPL12, SLC7A6OS, FADS2, SELPLG, CXCR4, GPR146, ZFP386, BCL11A, TRIM34A, RPS7, TLR9, PACSIN1, PAIP1, PGAM2 and JAKMIP1.

A yet further aspect of the invention relates to a kit of parts comprising means for detection of the above signature of dysfunction. Also provided is a kit of parts comprising means for detection of the signature of dysfunction, activation, activation and/or dysfunction, or memory as taught herein.

Another aspect of the invention provides a method for determining whether or not an immune cell has a dysfunctional immune phenotype and/or whether or not an immune cell would benefit from upregulation of an immune response, said method comprising: (a) determining in said immune cell the expression of POU2AF1, whereby expression of POU2AF1 indicates that the immune cell has a dysfunctional immune phenotype and/or would benefit from upregulation of an immune response; or (b) determining in said immune cell the expression of the signature of dysfunction as defined herein, whereby expression of the signature indicates that the immune cell has a dysfunctional immune phenotype and/or would benefit from upregulation of an immune response.

Also provided is a method for determining whether or not an immune cell has an activation, activation and/or dysfunction or memory immune phenotype and/or whether or not an immune cell would benefit from modulation (e.g., downregulation or upregulation) of an immune response, said method comprising: determining in said immune cell the expression of the signature of activation, activation and/or dysfunction, or memory, as defined herein, whereby expression of the signature indicates that the immune cell has respectively an activation, activation and/or dysfunction or memory immune phenotype and/or would benefit from modulation of an immune response.

A further aspect of the invention provides a method for determining whether or not a patient would benefit from a therapy aimed at reducing dysfunction of immune cells or a therapy aimed at upregulating of an immune response, the method comprising: (a) determining, in immune cells from said patient the expression of POU2AF1, whereby expression of POU2AF1 indicates that the patient will benefit from the therapy; or (b) determining, in immune cells from said patient the expression of the signature of dysfunction as defined above, whereby expression of the signature indicates the patient will benefit from the therapy.

Also provided is a method for determining whether or not a patient would benefit from a therapy aimed at modulating (e.g., reducing or increasing) activation, activation and/or dysfunction or memory phenotype of immune cells, or a therapy aimed at modulating (e.g. reducing or increasing) of an immune response, said method comprising determining, in immune cells from said patient the expression of the signature of activation, activation and/or dysfunction, or memory, as defined herein, whereby expression of the signature indicates that the patient will benefit from the therapy aimed at modulating respectively the activation, activation and/or dysfunction or memory phenotype of immune cells, or will benefit from the therapy aimed at modulating the immune response.

Another aspect of the invention relates to a method for determining the efficacy of a treatment of a patient with a therapy, said method comprising: (a) determining in immune cells from said patient the expression of POU2AF1 before and after said treatment and determining the efficacy of said therapy based thereon, whereby unchanged or increased expression of POU2AF1 indicates that the treatment should be adjusted; or (b) determining in immune cells from said patient the expression of the signature of dysfunction as defined above before and after said treatment and determining the efficacy of said therapy based thereon, whereby unchanged or increased expression of the signature indicates that the treatment should be adjusted.

Also provided is a method for determining the efficacy of a treatment of a patient with a therapy, said method comprising determining in immune cells from said patient the expression of the signature of activation, activation and/or dysfunction, or memory, as defined herein, before and after said treatment and determining the efficacy of said therapy based thereon, whereby unchanged or increased expression of the signature indicates that the treatment should be adjusted.

Another aspect of the invention provides a method for determining the suitability of a compound as a checkpoint inhibitor, said method comprising: (a) contacting an immune cell expressing POU2AF1 with said compound and determining whether or not said compound can affect the expression of POU2AF1 by said cell, whereby decreased expression indicates that the compound is suitable as a checkpoint inhibitor; or (b) contacting an immune cell expressing the signature of dysfunction as defined above with said compound and determining whether or not said compound can affect the expression of the signature by said cell, whereby decreased expression indicates that the compound is suitable as a checkpoint inhibitor.

Also provided is a method for determining the suitability of a compound as a checkpoint inhibitor, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable as a checkpoint inhibitor (e.g., whereby increased expression of the signature of activation indicates that the compound is suitable as a checkpoint inhibitor).

A further aspect of the invention provides a method for determining the suitability of a compound for reducing an dysfunctional immune phenotype and/or upregulating of an immune response, said method comprising: (a) contacting an immune cell expressing POU2AF1 with said compound and determining whether or not said compound can affect the expression of POU2AF1 by said cell, whereby decreased expression indicates that the compound is suitable for reducing dysfunctional immune phenotype and/or upregulating of an immune response; or (b) contacting an immune cell expressing the signature of dysfunction as defined above with said compound and determining whether or not said compound can affect the expression of the signature by said cell, whereby decreased expression indicates that the compound is suitable for reducing dysfunctional immune phenotype and/or upregulating of an immune response.

Also provided is a method for determining the suitability of a compound for modulating (e.g., reducing or increasing) activation, activation and/or dysfunction or memory phenotype of immune cells, and/or modulating (e.g., reducing or increasing) of an immune response, said method comprising contacting an immune cell expressing the signature of activation, activation and/or dysfunction, or memory, as defined herein, with said compound and determining whether or not said compound can affect the expression of the signature by said cell, whereby altered expression indicates that the compound is suitable for modulating respectively the activation, activation and/or dysfunction or memory phenotype of immune cells, and/or modulating of the immune response.

A yet another aspect of the invention provides a method for stratification of immune cells into one or more cell populations comprising at least a first cell population having a comparatively more dysfunctional immune phenotype and a second population having a comparatively less dysfunctional immune phenotype, comprising: (a) determining in said immune cells the expression of POU2AF1, and allotting cells having no or comparatively lower expression of POU2AF1 into said second population, and cells having comparatively higher expression of POU2AF1 into said first population; or (b) determining in said immune cells the expression of the signature of dysfunction as defined above, and allotting cells having no or comparatively lower expression of said signature into said second population, and cells having comparatively higher expression of said signature into said first population.

Also provided is a method for stratification of immune cells into one or more cell populations comprising at least a first cell population having a comparatively more activation, activation and/or dysfunction or memory phenotype and a second population having a comparatively less activation, activation and/or dysfunction or memory phenotype, said method comprising determining in said immune cells the expression of the signature of activation, activation and/or dysfunction, or memory, as defined herein, and allotting cells having no or comparatively lower expression of said signature into said second population, and cells having comparatively higher expression of said signature into said first population.

Also provided is a method for stratification of immune cells into one or more cell populations comprising at least a first cell population having a comparatively more activation, activation and/or dysfunction or memory phenotype and a second population having a comparatively less activation, activation and/or dysfunction or memory phenotype, said method comprising determining in said immune cells the expression of the signature of activation, activation and/or dysfunction, or memory, as defined herein, and allotting cells having no or comparatively lower expression of said signature into said second population, and cells having comparatively higher expression of said signature into said first population.

A yet another aspect provides a method of isolating an immune cell as taught herein comprising binding of an affinity ligand to a signature gene expressed on the surface of the immune cell.

A further aspect provides a method of treating a subject in need thereof, comprising administering to said subject an agent capable of modulating the immune cell as taught herein.

A further aspect provides a method of treatment comprising administering one or more checkpoint inhibitors to a patient in need thereof, wherein immune cells obtained from the patient have a gene signature as taught herein, such as the gene signature of dysfunction as taught herein.

### Adoptive Cell Therapies

The present invention also contemplates use of the CRISPR-Cas system described herein, e.g. C2cl effector protein systems, to modify cells for adoptive therapies.

As used herein, “ACT”, “adoptive cell therapy” and “adoptive cell transfer” may be used interchangeably. In certain embodiments, adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73). In certain embodiments, allogenic immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

In some embodiments, the invention described herein relates to a method for adoptive immunotherapy, in which T cells are edited ex vivo by CRISPR to modulate at least one gene and subsequently administered to a patient in need thereof. In some embodiments, the CRISPR editing comprising knocking-out or knocking-down the expression of at least one target gene in the edited T cells. In some embodiments, in addition to modulating the target gene, the T cells are also edited ex vivo by CRISPR to (1) knock-in an exogenous gene encoding a chimeric antigen receptor (CAR) or a T-cell receptor (TCR), (2) knock-out or knock-down expression of an immune checkpoint receptor, (3) knock-out or knock-down expression of an endogenous TCR, (4) knock-out or knock-down expression of a human leukocyte antigen class I (HLA-I) proteins, and/or (5) knock-out or knock-down expression of an endogenous gene encoding an antigen targeted by an exogenous CAR or TCR.

In some embodiments, the T cells are contacted ex vivo with an adeno-associated virus (AAV) vector encoding a CRISPR effector protein, and a guide molecule comprising a guide sequence hybridizable to a target sequence, a tracr mate sequence, and a tracr sequence hybridizable to the tracr mate sequence. In some embodiments, the T cells are contacted ex vivo (e.g., by electroporation) with a ribonucleoprotein (RNP) comprising a CRISPR effector protein complexed with a guide molecule, wherein the guide molecule comprising a guide sequence hybridizable to a target sequence, a tracr mate sequence, and a tracr sequence hybridizable to the tracr mate sequence. See Rupp et al., Scientific Reports 7:737 (2017); Liu et al., Cell Research 27:154-157 (2017). In some embodiments, the T cells are contacted ex vivo (e.g., by electroporation) with an mRNA encoding a CRISPR effector protein, and a guide molecule comprising a guide sequence hybridizable to a target sequence, a tracr mate sequence, and a tracr sequence hybridizable to the tracr mate sequence. See Eyquem et al., Nature 543:113-117 (2017). In some embodiments, the T cells are not contacted ex vivo with a lentivirus or retrovirus vector.

In some embodiments, the method comprises editing T cells ex vivo by CRISPR to knock-in an exogenous gene encoding a CAR, thereby allowing the edited T cells to recognize cancer cells based on the expression of specific proteins located on the cell surface. In some embodiments, T cells are edited ex vivo by CRISPR to knock-in an exogenous gene encoding a TCR, thereby allowing the edited T cells to recognize proteins derived from either the surface or inside of the cancer cells. In some embodiments, the method comprising providing an exogenous CAR-encoding or TCR-encoding sequence as a donor sequence, which can be integrated by homology-directed repair (HDR) into a genomic locus targeted by a CRISPR guide sequence. In some embodiments, targeting the exogenous CAR or TCR to an endogenous TCR a constant (TRAC) locus can reduce tonic CAR signaling and facilitate effective internalization and re-expression of the CAR following single or repeated exposure to antigen, thereby delaying effector T-cell differentiation and exhaustion. See Eyquem et al., Nature 543:113-117 (2017).

In some embodiments, the method comprises editing T cells ex vivo by CRISPR to block one or more immune checkpoint receptors to reduce immunosuppression by cancer cells. In some embodiments, T cells are edited ex vivo by CRISPR to knock-out or knock-down an endogenous gene involved in the programmed death-1 (PD-1) signaling pathway, such as PD-1 and PD-L1. In some embodiments, T cells are edited ex vivo by CRISPR to mutate the Pdcd1 locus or the CD274 locus. In some embodiments, T cells are edited ex vivo by CRISPR using one or more guide sequences targeting the first exon of PD-1. See Rupp et al., Scientific Reports 7:737 (2017); Liu et al., Cell Research 27:154-157 (2017).

In some embodiments, the method comprises editing T cells ex vivo by CRISPR to eliminate potential alloreactive TCRs to allow allogeneic adoptive transfer. In some embodiments, T cells are edited ex vivo by CRISPR to knock-out or knock-down an endogenous gene encoding a TCR (e.g., an αβ TCR) to avoid graft-versus-host-disease (GVHD). In some embodiments, T cells are edited ex vivo by CRISPR to mutate the TRAC locus. In some embodiments, T cells are edited ex vivo by CRISPR using one or more guide sequences targeting the first exon of TRAC. See Liu et al., Cell Research 27:154-157 (2017). In some embodiments, the method comprises use of CRISPR to knock-in an exogenous gene encoding a CAR or a TCR into the TRAC locus, while simultaneously knocking-out the endogenous TCR (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous TCR promoter.

In some embodiments, the method comprises editing T cells ex vivo by CRISPR to knock-out or knock-down an endogenous gene encoding an HLA-I protein to minimize immunogenicity of the edited T cells. In some embodiments, T cells are edited ex vivo by CRISPR to mutate the beta-2 microglobulin (B2M) locus. In some embodiments, T cells are edited ex vivo by CRISPR using one or more guide sequences targeting the first exon of B2M. See Liu et al., Cell Research 27:154-157 (2017). In some embodiments, the method comprises use of CRISPR to knock-in an exogenous gene encoding a CAR or a TCR into the B2M locus, while simultaneously knocking-out the endogenous B2M (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous B2M promoter.

In some embodiments, the method comprises editing T cells ex vivo by CRISPR to knock-out or knock-down an endogenous gene encoding an antigen targeted by an exogenous CAR or TCR. In some embodiments, the T cells are edited ex vivo by CRISPR to knock-out or knock-down the expression of a tumor antigen selected from human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI) (see WO2016/011210). In some embodiments, the T cells are edited ex vivo by CRISPR to knock-out or knock-down the expression of an antigen selected from B cell maturation antigen (BCMA), transmembrane activator and CAML Interactor (TACI), or B-cell activating factor receptor (BAFF-R), CD38, CD138, CS-1, CD33, CD26, CD30, CD53, CD92, CD100, CD148, CD150, CD200, CD261, CD262, or CD362 (see WO2017/011804).

Aspects of the invention accordingly involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; and, Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Autologous T cells engineered to express chimeric antigen receptors (CARs) against leukemia antigens such as CD19 on B cells have shown promising results for the treatment of relapsed or refractory B-cell malignancies. However, a subset of cancer patients especially heavily pretreated cancer patients could be unable to receive this highly active therapy because of failed expansion. Moreover, it is still a challenge to manufacture an effective therapeutic product for infant cancer patients due to their small blood volume. On the other hand, the inherent characters of autologous CAR-T cell therapy including personalized autologous T cell manufacturing and widely “distributed” approach result in the difficulty of industrialization of autologous CAR-T cell therapy. Universal CD19-specific CAR-T cell (UCART019), derived from one or more healthy unrelated donors but could avoid graft-versus-host-disease (GVHD) and minimize their immunogenicity, is undoubtedly an alternative option to address above-mentioned issues. Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects. Han et. al (clinicaltrials, A Study Evaluating UCART019 in Patients with Relapsed or Refactory CD19+ Lukemia and Lymphoma) have generated gene-disrupted allogeneic CD19-directed BBζ CAR-T cells (termed UCART019) by combining the lentiviral delivery of CAR and CRISPR RNA electroporation to disrupt endogenous TCR and B2M genes simultaneously and will test whether it can evade host-mediated immunity and deliver antileukemic effects without GVHD.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD 11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS) (SEQ ID NO:2)). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly, Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

The CRISPR systems disclosed herein may be used for targeting an antigen to be targeted in adoptive cell therapy. In certain embodiments, an antigen (such as a tumor antigen) to be targeted in adoptive cell therapy (such as TIL, CAR, or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; trophoblast glycoprotein (TPBG); αvβó integrin, B7-H3; B7-H6; CD20; CD44; chondroitin sulfate proteoglycan 4 (CSPG4), bDGalpNAc(1-4)[aNeu5Ac(2-8)aNeu5Ac(2-3)]bDGalp(1-4)bDGlcp(1-1)Cer (GD2), aNeu5Ac(2-8)aNeu5Ac(2-3)bDGalp(1-4)bDGlcp(1-1)Cer (GD3); human leukocyte antigen A1 MAGE family member A1 (HLA-A1⁺MAGEA1); human leukocyte antigen A2 MAGE family member A1 (HLA-A2⁺MAGEA1); human leukocyte antigen A3 MAGE family member A1 (HLA-A3⁺MAGEA1); MAGEA1; human leukocyte antigen A1 New York Esophageal Squamous Cell Carcinoma 1 (FILA-A1⁺NY-ESO-1); human leukocyte antigen A2 New York Esophageal Squamous Cell Carcinoma 1 (HLA-A2⁺NY-ESO-1), lambda light chain, kappa light chain, tumor endothelial marker 5 (TEM5), tumor endothelial marker 7 (TEM7), tumor endothelial marker 8 (TEM8), TEM5, TEM7, TEM8, IFN-inducible p78, melanotransferrin (p97), human kallikrein (huK2), Ax1, ROR2, FKBP11, KAMP3, ITGA8, FCRL5, LAGA-1, CD133, cD34, EBV nuclear antigen-1 (EBNA1), latent membrane protein 1 (LMP1) and LMP2A, CD75, gp100, MICA, MICB, MART1, carcinoembryonic antigen, CA-125, MAGEC2, CTAG2, CTAG1, pd-12, CLA, CD142, CD73, CD49c, CD66c, CD104, CD318, TSPAN8, CLEC14, human immunodeficiency virus 1 (HIV-1) reverse transcriptase (RT), Cd16, BLTA, IL-2, IL-7, IL-15, IL-21, IL-12, CCR4, CCR2b, Heparanase, CD137L, LEM, and Bcl-2, Msln, Cd8, IL-15, 4-1BBL, OX40L, 4-IBB, cd95, cd27, HVENM, CXCR4; and any combination thereof. In some example, the antigen to be targeted may be CXCR. In some examples, the antigen to be targeted may be PD-1.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

In some embodiments, the target antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatit s B (e.g., HBV core and surface antigens (HBVc, HBVs)), hepatitis C (HCV), Epstein-Ban* virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-i, HIN-1, HIN-IL CMN, EBN or HPN. In some embodiments, the target protein is a bacterial antigen or other pathogenic antigen, such as Mycobacterium tuberculosis (MT) antigens, trypanosome, e.g., Tiypansoma cruzi (T. cruzi), antigens such as surface antigen (TSA), or malaria antigens. Specific viral antigen or epitopes or other pathogenic antigens or peptide epitopes are known (see e.g., Addo et al. (2007) PLoS ONE, 2, e321; Anikeeva et al. (2009) Clin Immunol, 130, 98-109). In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (HBV), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen. In some embodiments, the viral antigen is an HPV antigen, which, in some cases, can lead to a greater risk of developing cervical and/or head and neck cancers. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigens (e.g., seroreactive regions of the E1, E2, E6 or E7 proteins of HPV-16, see e.g. U.S. Pat. No. 6,531,127) or an HPV-18 antigens (e.g., seroreactive regions of the LI and/or L2 proteins of HPV-18, such as described in U.S. Pat. No. 5,840,306).

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or a hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, such as EBNA-1, LMP-1 and LMP-2A. In some embodiments, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA or EBV-VCA. In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the viral antigen is a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers.

In one aspect, the present invention provides a treatment for tumors of the central nervous system, particularly induced by neurofibromatosis type 1 (NF1) neurogenetic conditions. Individuals with NF1 are born with a germline mutation in the NF1 gene, but may develop numerous distinct neurological problems, ranging from autism and attention deficit to brain and peripheral nerve sheath tumors. The present invention may be used to develop a patient-specific disease model and to study induced pluripotent stem cell (iPSC)-derived disease relevant cells in an isogenic background. Embryonic stem cell (ESC)-like cells, also known as induced pluripotent stem cell or iPSC, can be generated from skin or blood cells in adult patients. Recent research efforts have started to develop culture protocols that differentiate iPSCs into a variety of cell types in the central and peripheral nervous system (CNS and PNS), which are affected in NF1 patients. The CRISPR C2cl system of this invention may be used to genetically edit the specific disease genes either by repairing the existing mutant genes or creating new mutations. In order to position at the forefront of NF1 research, it will be important for the Gilbert Family Neurofibromatosis Institute (GFNI) at the Children's National Medical Center to explore these recent exciting research developments, to systematically develop patient-specific human NF1 disease models, and to provide a tool for drug screening and evaluation on the individual NF patients.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction). Dosing in CAR T cell therapies may for example involve administration of from 106 to 109 cells/kg, with or without a course of lymphodepletion, for example with cyclophosphamide.

A person with ordinary skills in the art may use a C2cl CRISPR system described herein in a similar system as described above. With respect to the C2cl protein, the C2cl CRISPR system may recognize a PAM sequence that is a T-rich sequence. In some embodiments, the PAM sequence is 5′ TTN 3′ or 5′ ATTN 3′, wherein N is any nucleotide. In some embodiments, the C2cl CRISPR system introduces one or more staggered double strand breaks (DSBs) with a 5′ overhang to the target gene. In particular embodiments, the 5′ overhang is 7 nt. In some embodiments, the C2cl CRISPR system introduces a template DNA sequence at the staggered DSB via HR or NHEJ. In some particular embodiments, the C2cl CRISPR system comprises a catalytically inactivated C2cl protein associated with a functional domain that modifies the target gene. In a particular embodiment, the C2cl CRISPR system introduces a single mutation. In another particular embodiment, the C2cl CRISPR system introduces a single nucleotide modification to the transcript of the target gene.

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 104-109 cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of the managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing with a CRISPR-Cas system as described herein may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). For example, immunoresponsive cells may be edited to delete expression of some or all of the class of HLA type II and/or type I molecules, or to knockout selected genes that may inhibit the desired immune response, such as the PD1 gene.

Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

### Identifying Immunomodulators

A further aspect of the invention relates to a method for identifying an immunomodulator capable of modulating one or more phenotypic aspects of an immune cell or immune cell population as disclosed herein, comprising: a) applying a candidate immunomodulator to the immune cell or immune cell population; b) detecting modulation of one or more phenotypic aspects of the immune cell or immune cell population by the candidate immunomodulator, thereby identifying the immunomodulator.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “immunomodulator” broadly encompasses any condition, substance or agent capable of modulating one or more phenotypic aspects of an immune cell or immune cell population as disclosed herein. Such conditions, substances or agents may be of physical, chemical, biochemical and/or biological nature. The term “candidate immunomodulator” refers to any condition, substance or agent that is being examined for the ability to modulate one or more phenotypic aspects of an immune cell or immune cell population as disclosed herein in a method comprising applying the candidate immunomodulator to the immune cell or immune cell population (e.g., exposing the immune cell or immune cell population to the candidate immunomodulator or contacting the immune cell or immune cell population with the candidate immunomodulator) and observing whether the desired modulation takes place.

Immunomodulators may include any potential class of biologically active conditions, substances or agents, such as for instance antibodies, proteins, peptides, nucleic acids, oligonucleotides, small molecules, or combinations thereof.

By means of example but without limitation, immunomodulators can include low molecular weight compounds, but may also be larger compounds, or any organic or inorganic molecule effective in the given situation, including modified and unmodified nucleic acids such as antisense nucleic acids, RNAi, such as siRNA or shRNA, CRISPR/Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments, the agent is a small molecule having a chemical moiety. Agents can be known to have a desired activity and/or property, or can be selected from a library of diverse compounds.

In certain embodiments, an immunomodulator may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-1β, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signalling molecules, such as tumour necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as immunomodulators include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Altering Expression Using Immunomodulators

In certain embodiments, an immunomodulator may alter expression and/or activity of one or more endogenous genes of the CAR T cells. The term “altered expression” denotes that the modification of the immune cell alters, i.e., changes or modulates, the expression of the recited gene(s) or polypeptides(s). The term “altered expression” encompasses any direction and any extent of said alteration. Hence, “altered expression” may reflect qualitative and/or quantitative change(s) of expression, and specifically encompasses both increase (e.g., activation or stimulation) or decrease (e.g., inhibition) of expression.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures of the present may be used to screen for drugs that induce or reduce the signature in immune cells as described herein. The signature may be used for GE-HTS. In certain embodiments, pharmacological screens may be used to identify drugs that selectively reduce or increase activity of suppressive immune cells. In certain embodiments, drugs that selectively activate or repress suppressive T cells are used for treatment of a cancer patient or a patient suffering from an autoimmune disease.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, cmap can be used to screen for small molecules capable of modulating a signature of the present invention in silico.

Any one or more of the several successive molecular mechanisms involved in the expression of a given gene or polypeptide may be targeted by the immune cell modification as intended herein. Without limitation, these may include targeting the gene sequence (e.g., targeting the polypeptide-encoding, non-coding and/or regulatory portions of the gene sequence), the transcription of the gene into RNA, the polyadenylation and where applicable splicing and/or other post-transcriptional modifications of the RNA into mRNA, the localization of the mRNA into cell cytoplasm, where applicable other post-transcriptional modifications of the mRNA, the translation of the mRNA into a polypeptide chain, where applicable post-translational modifications of the polypeptide, and/or folding of the polypeptide chain into the mature conformation of the polypeptide. For compartmentalized polypeptides, such as secreted polypeptides and transmembrane polypeptides, this may further include targeting trafficking of the polypeptides, i.e., the cellular mechanism by which polypeptides are transported to the appropriate sub-cellular compartment or organelle, membrane, e.g. the plasma membrane, or outside the cell.

Hence, “altered expression” may particularly denote altered production of the recited gene products by the modified immune cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

In certain embodiments, an immunomodulator may be or may result in a genetic modification (e.g., mutation, editing, transgenesis, or combinations thereof) of an immune cell, for example, a genetic perturbation, such as a knock-out (i.e., resulting in a complete absence of expression and/or activity) of one or more endogenous genes/gene products, or a knock-down (i.e., resulting in a partial absence of expression and/or activity) of one or more endogenous genes/gene products, or another type of genetic modification modulating the expression and/or activity of one or more endogenous genes/gene products, or for example, introduction of one or more transgenes, such as one or more transgenes encoding one or more gene products. Such transgene may be suitably operably linked to suitable regulatory sequences, e.g., may be comprised in an expression cassette or an expression vector comprising suitable regulatory sequences, or may be configured to become operably linked to suitable regulatory sequences once inserted into the genetic material (e.g., genome) of the immune cell.

Any types of mutations achieving the intended effects are contemplated herein. For example, suitable mutations may include deletions, insertions, and/or substitutions. The term “deletion” refers to a mutation wherein one or more nucleotides, typically consecutive nucleotides, of a nucleic acid are removed, i.e., deleted, from the nucleic acid. The term “insertion” refers to a mutation wherein one or more nucleotides, typically consecutive nucleotides, are added, i.e., inserted, into a nucleic acid. The term “substitution” refers to a mutation wherein one or more nucleotides of a nucleic acid are each independently replaced, i.e., substituted, by another nucleotide.

In certain embodiments, a mutation may introduce a premature in-frame stop codon into the open reading frame (ORF) encoding a gene product. Such premature stop codon may lead to production of a C-terminally truncated form of said polypeptide (this may preferably affect, such as diminish or abolish, some or all biological function(s) of the polypeptide) or, especially when the stop codon is introduced close to (e.g., about 20 or less, or about 10 or less amino acids downstream of) the translation initiation codon of the ORF, the stop codon may effectively abolish the production of the polypeptide. Various ways of introducing a premature in-frame stop codon are apparent to a skilled person. For example, but without limitation, a suitable insertion, deletion or substitution of one or more nucleotides in the ORF may introduce the premature in-frame stop codon.

In other embodiments, a mutation may introduce a frame shift (e.g., +1 or +2 frame shift) in the ORF encoding a gene product. Typically, such frame shift may lead to a previously out-of-frame stop codon downstream of the mutation becoming an in-frame stop codon. Hence, such frame shift may lead to production of a form of the polypeptide having an alternative C-terminal portion and/or a C-terminally truncated form of said polypeptide (this may preferably affect, such as diminish or abolish, some or all biological function(s) of the polypeptide) or, especially when the mutation is introduced close to (e.g., about 20 or less, or about 10 or less amino acids downstream of) the translation initiation codon of the ORF, the frame shift may effectively abolish the production of the polypeptide. Various ways of introducing a frame shift are apparent to a skilled person. For example, but without limitation, a suitable insertion or deletion of one or more (not multiple of 3) nucleotides in the ORF may lead to a frame shift.

In further embodiments, a mutation may delete at least a portion of the ORF encoding a gene product. Such deletion may lead to production of an N-terminally truncated form, a C-terminally truncated form and/or an internally deleted form of said polypeptide (this may preferably affect, such as diminish or abolish, some or all biological function(s) of the polypeptide). Preferably, the deletion may remove about 20% or more, or about 50% or more of the ORF's nucleotides. Especially when the deletion removes a sizeable portion of the ORF (e.g., about 50% or more, preferably about 60% or more, more preferably about 70% or more, even more preferably about 80% or more, still more preferably about 90% or more of the ORF's nucleotides) or when the deletion removes the entire ORF, the deletion may effectively abolish the production of the polypeptide. The skilled person can readily introduce such deletions.

In further embodiments, a mutation may delete at least a portion of a gene promoter, leading to impaired transcription of the gene product.

In certain other embodiments, a mutation may be a substitution of one or more nucleotides in the ORF encoding a gene product resulting in substitution of one or more amino acids of the polypeptide. Such mutation may typically preserve the production of the polypeptide, and may preferably affect, such as diminish or abolish, some or all biological function(s) of the polypeptide. The skilled person can readily introduce such substitutions.

In certain preferred embodiments, a mutation may abolish native splicing of a pre-mRNA encoding a gene product. In the absence of native splicing, the pre-mRNA may be degraded, or the pre-mRNA may be alternatively spliced, or the pre-mRNA may be spliced improperly employing latent splice site(s) if available. Hence, such mutation may typically effectively abolish the production of the polypeptide's mRNA and thus the production of the polypeptide. Various ways of interfering with proper splicing are available to a skilled person, such as for example but without limitation, mutations which alter the sequence of one or more sequence elements required for splicing to render them inoperable, or mutations which comprise or consist of a deletion of one or more sequence elements required for splicing. The terms “splicing”, “splicing of a gene”, “splicing of a pre-mRNA” and similar as used herein are synonymous and have their art-established meaning. By means of additional explanation, splicing denotes the process and means of removing intervening sequences (introns) from pre-mRNA in the process of producing mature mRNA. The reference to splicing particularly aims at native splicing such as occurs under normal physiological conditions. The terms “pre-mRNA” and “transcript” are used herein to denote RNA species that precede mature mRNA, such as in particular a primary RNA transcript and any partially processed forms thereof. Sequence elements required for splicing refer particularly to cis elements in the sequence of pre-mRNA which direct the cellular splicing machinery (spliceosome) towards correct and precise removal of introns from the pre-mRNA. Sequence elements involved in splicing are generally known per se and can be further determined by known techniques including inter alia mutation or deletion analysis. By means of further explanation, “splice donor site” or “5′ splice site” generally refer to a conserved sequence immediately adjacent to an exon-intron boundary at the 5′ end of an intron. Commonly, a splice donor site may contain a dinucleotide GU, and may involve a consensus sequence of about 8 bases at about positions +2 to −6. “Splice acceptor site” or “3′ splice site” generally refers to a conserved sequence immediately adjacent to an intron-exon boundary at the 3′ end of an intron. Commonly, a splice acceptor site may contain a dinucleotide AG, and may involve a consensus sequence of about 16 bases at about positions −14 to +2.

In certain embodiments, the one or more modulating agents may be a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

### Genetic Modifying Agents

Described throughout this specification are methods of modifying cells, such as T cells, such that the express or do not express specific genes or gene products. In certain embodiments, the one or more modulating agents may be a genetic modifying agent. The genetic modifying agent may comprise a CRISPR system, a zinc finger nuclease system, a TALEN, a meganuclease or RNAi system.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words, samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related, or are only partially structurally related. An “orthologue” of a protein as used herein is a protein of a different species which performs the same or a similar function as the protein it is an orthologue of: Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiments, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI.10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment, the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments, the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiments, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green flourescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends a guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (40HT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogren receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for about 5 minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, nonuniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Also described herein is the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz' (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142). Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023, which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprises one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional Application No. 62/351,662 filed on Jun. 17, 2016 and U.S. Provisional Application No. 62/376,377 filed on Aug. 17, 2016. Reference is also made to U.S. Provisional Application No. 62/351,803 filed on Jun. 17, 2016. Reference is also made to U.S. Provisional entitled “Novel CRISPR Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytindine or adenosine deaminases that mediate endogenous editing via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenonsine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

A further aspect relates to an isolated cell obtained or obtainable from the methods described herein comprising the composition described herein or progeny of said modified cell, preferably wherein said cell comprises a hypoxanthine or a guanine in replacement of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:

Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);

RNA-guided editing of bacterial genomes using CRISPR-Cas systems. Jiang W., Bikard D., Cox D., Zhang F, Marraffini L A. Nat Biotechnol March; 31(3):233-9 (2013);

One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);

Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. Aug. 22; 500(7463):472-6. doi: 10.1038/Nature12466. Epub 2013 Aug. 23 (2013);

Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);

DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);

Genome engineering using the CRISPR-Cas9 system. Ran, F A., Hsu, P D., Wright, J., Agarwala, V., Scott, D A., Zhang, F. Nature Protocols November; 8(11):2281-308 (2013-B);

Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);

Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell Feb. 27, 156(5):935-49 (2014);

Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. Apr. 20. doi: 10.1038/nbt.2889 (2014);

CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);

Development and Applications of CRISPR-Cas9 for Genome Engineering, Hsu P D, Lander E S, Zhang F., Cell. June 5; 157(6):1262-78 (2014).

Genetic screens in human cells using the CRISPR-Cas9 system, Wang T, Wei J J, Sabatini D M, Lander E S., Science. January 3; 343(6166): 80-84. doi:10.1126/science.1246981 (2014);

Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. Dec; 32(12):1262-7 (2014);

In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);

Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).

A split-Cas9 architecture for inducible genome editing and transcription modulation, Zetsche B, Volz S E, Zhang F., (published online 2 Feb. 2015) Nat Biotechnol. February; 33(2):139-42 (2015);

Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and

In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).

Shalem et al., “High-throughput functional genomics using CRISPR-Cas9,” Nature Reviews Genetics 16, 299-311 (May 2015).

Xu et al., “Sequence determinants of improved CRISPR sgRNA design,” Genome Research 25, 1147-1157 (August 2015).

Parnas et al., “A Genome-wide CRISPR Screen in Primary Immune Cells to Dissect Regulatory Networks,” Cell 162, 675-686 (Jul. 30, 2015).

Ramanan et al., CRISPR-Cas9 cleavage of viral DNA efficiently suppresses hepatitis B virus,” Scientific Reports 5:10833. doi: 10.1038/srep10833 (Jun. 2, 2015)

Nishimasu et al., Crystal Structure of Staphylococcus aureus Cas9,” Cell 162, 1113-1126 (Aug. 27, 2015)

BCL11A enhancer dissection by Cas9-mediated in situ saturating mutagenesis, Canver et al., Nature 527(7577):192-7 (Nov. 12, 2015) doi: 10.1038/nature15521. Epub 2015 September 16.

Cpf1 Is a Single RNA-Guided Endonuclease of a Class 2 CRISPR-Cas System, Zetsche et al., Cell 163, 759-71 (Sep. 25, 2015).

Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.

Rationally engineered Cas9 nucleases with improved specificity, Slaymaker et al., Science 2016 Jan. 1 351(6268): 84-88 doi: 10.1126/science.aad5227. Epub 2015 Dec. 1.

Gao et al., “Engineered Cpf1 Enzymes with Altered PAM Specificities,” bioRxiv 091611; doi: http://dx.doi.org/10.1101/091611 (Dec. 4, 2016).

Cox et al., “RNA editing with CRISPR-Cas13,” Science. 2017 Nov. 24; 358(6366):1019-1027. doi: 10.1126/science.aaq0180. Epub 2017 Oct. 25.

each of which is incorporated herein by reference, may be considered in the practice of the instant invention, and discussed briefly below:

Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.

Konermann et al. (2013) addressed the need in the art for versatile and robust technologies that enable optical and chemical modulation of DNA-binding domains based CRISPR Cas9 enzyme and also Transcriptional Activator Like Effectors.

Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.

Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.

Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Platt et al. established a Cre-dependent Cas9 knockin mouse. The authors demonstrated in vivo as well as ex vivo genome editing using adeno-associated virus (AAV)−, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al. (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an online tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al. relates to multiplex screening by demonstrating that a genome-wide in vivo CRISPR-Cas9 screen in mice reveals genes regulating lung metastasis.

Ran et al. (2015) relates to SaCas9 and its ability to edit genomes and demonstrates that one cannot extrapolate from biochemical assays.

Shalem et al. (2015) described ways in which catalytically inactive Cas9 (dCas9) fusions are used to synthetically repress (CRISPRi) or activate (CRISPRa) expression, showing advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.

Ramanan et al. (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.

Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Canver et al. (2015) demonstrated a CRISPR-Cas9-based functional investigation of non-coding genomic elements. The authors developed pooled CRISPR-Cas9 guide RNA libraries to perform in situ saturating mutagenesis of the human and mouse BCL11A enhancers which revealed critical features of the enhancers.

Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.

Shmakov et al. (2015) reported three distinct Class 2 CRISPR-Cas systems. Two system CRISPR enzymes (C2cl and C2c3) contain RuvC-like endonuclease domains distantly related to Cpf1. Unlike Cpf1, C2cl depends on both crRNA and tracrRNA for DNA cleavage. The third enzyme (C2c2) contains two predicted HEPN RNase domains and is tracrRNA independent.

Slaymaker et al. (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

Cox et al. (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein may be designed for use with Cas13, a type II nuclease that does not make use of tracrRNA. Orthologs of Cas13 have been identified in different bacterial species as described herein. Further, type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung, Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 A1 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 A1 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 A1 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 A1 (U.S. application Ser. No. 14/183,429); US 2015-0184139 A1 (U.S. application Ser. No. 14/324,960); U.S. application Ser. No. 14/054,414; European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 15, PROTECTED GUIDE RNAS (PGRNAS); U.S. application 62/091,455, filed, 12-Dec. 14, PROTECTED GUIDE RNAS (PGRNAS); U.S. application 62/096,708, 24-Dec. 14, PROTECTED GUIDE RNAS (PGRNAS); U.S. applications 62/091,462, 12-Dec. 14, 62/096,324, 23-Dec. 14, 62/180,681, 17 Jun. 2015, and 62/237,496, 5 Oct. 2015, DEAD GUIDES FOR CRISPR TRANSCRIPTION FACTORS; U.S. application 62/091,456, 12-Dec. 14 and 62/180,692, 17-Jun. 2015, ESCORTED AND FUNCTIONALIZED GUIDES FOR CRISPR-CAS SYSTEMS; U.S. application 62/091,461, 12-Dec. 14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec. 14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec. 14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec. 14, 62/181,641, 18 Jun. 2015, and 62/181,667, 18 Jun. 2015, RNA-TARGETING SYSTEM; U.S. application 62/096,656, 24-Dec. 14 and 62/181,151, 17 Jun. 2015, CRISPR HAVING OR ASSOCIATED WITH DESTABILIZATION DOMAINS; U.S. application 62/096,697, 24-Dec. 14, CRISPR HAVING OR ASSOCIATED WITH AAV; U.S. application 62/098,158, 30-Dec. 14, ENGINEERED CRISPR COMPLEX INSERTIONAL TARGETING SYSTEMS; U.S. application 62/151,052, 22-Apr. 15, CELLULAR TARGETING FOR EXTRACELLULAR EXOSOMAL REPORTING; U.S. application 62/054,490, 24-Sep. 14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep. 14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4-Dec. 14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep. 14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep. 14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep. 14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep. 14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec. 14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep. 14, FUNCTIONAL SCREENING WITH OPTIMIZED FUNCTIONAL CRISPR-CAS SYSTEMS; U.S. application 62/087,546, 4-Dec. 14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec. 14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19-Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5-Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15-Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016/161516. WO2016/161516 describes efficient transduction of polypeptide cargos using synthetic peptides comprising an endosome leakage domain (ELD) operably linked to a cell penetrating domain (CPD), to a histidine-rich domain and a CPD. Similarly, these polypeptides can be used for the delivery of CRISPR-effector based RNPs in eukaryotic cells.

### Tale Systems

As disclosed herein, editing can be made by way of the transcription activator-like effector nucleases (TALENs) system. Transcription activator-like effectors (TALEs) can be engineered to bind practically any desired DNA sequence. Exemplary methods of genome editing using the TALEN system can be found, for example, in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P. Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments, the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

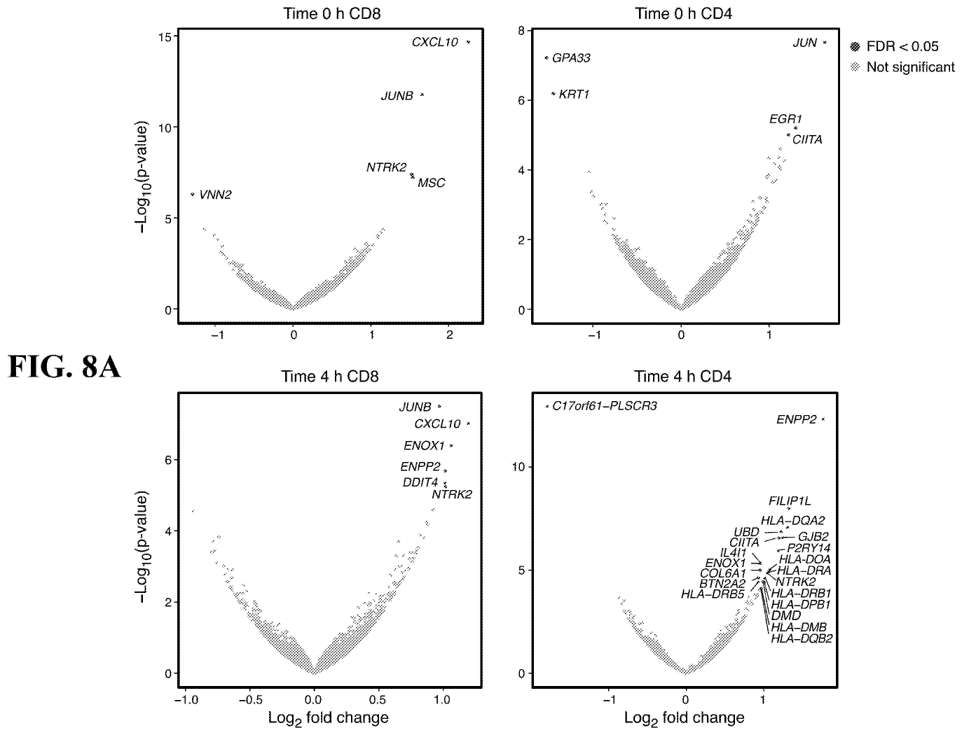

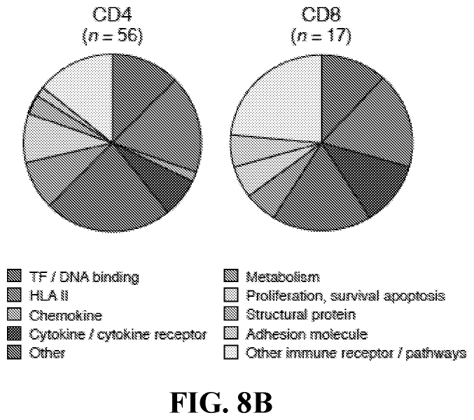

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID No. 3)
M D P I R S R T P S P A R E L L S G P Q P D G V
Q P T A D R G V S P P A G G P L D G L P A R R T
M S R T R L P S P P A P S P A F S A D S F S D L
L R Q F D P S L F N T S L F D S L P P F G A H H
T E A A T G E W D E V Q S G L R A A D A P P P T
M R V A V T A A R P P R A K P A P R R R A A Q P
S D A S P A A Q V D L R T L G Y S Q Q Q Q E K I
K P K V R S T V A Q H H E A L V G H G F T H A H
I V A L S Q H P A A L G T V A V K Y Q D M I A A
L P E A T H E A I V G V G K Q W S G A R A L E A
L L T V A G E L R G P P L Q L D T G Q L L K I A
K R G G V T A V E A V H A W R N A L T G A P L N

An exemplary amino acid sequence of a C-terminal capping region is:

(SEQ ID No. 4)
R P A L E S I V A Q L S R P D P A L A A L T N D
H L V A L A C L G G R P A L D A V K K G L P H A
P A L I K R T N R R I P E R T S H R V A D H A Q
V V R V L G F F Q C H S H P A Q A F D D A M T Q
F G M S R H G L L Q L F R R V G V T E L E A R S
G T L P P A S Q R W D R I L Q A S G M K R A K P
S P T S T Q T P D Q A S L H A F A D S L E R D L
D A P S P M H E G D Q T R A S

As used herein, the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer programs for carrying out alignments, like the GCG Wisconsin Bestfit package, may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID), SID4X domain or a Krüppel-associated box (KRAB), or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### ZN-Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Transcriptional Activation/Repression

In certain embodiments, an immunomodulator may comprise (i) a DNA-binding portion configured to specifically bind to the endogenous gene and (ii) an effector domain mediating a biological activity.

In certain embodiments, the DNA-binding portion may comprise a zinc finger protein or DNA-binding domain thereof, a transcription activator-like effector (TALE) protein or DNA-binding domain thereof, or an RNA-guided protein or DNA-binding domain thereof.

In certain embodiments, the DNA-binding portion may comprise (i) Cas9 or Cpf1 or any Cas protein described herein modified to eliminate its nuclease activity, or (ii) DNA-binding domain of Cas9 or Cpf1 or any Cas protein described herein.

In some embodiments, the effector domain may be a transcriptional inhibitor (i.e., a repressor domain), such as an mSin interaction domain (SID), SID4X domain or a Krüppel-associated box (KRAB), or fragments of the KRAB domain. In some embodiments, the effector domain may be an enhancer of transcription (i.e. an activation domain), such as the VP16, VP64 or p65 activation domain. In some embodiments, the nucleic acid binding portion may be linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal. In some embodiments, the effector domain may be a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Antibody Drug Conjugate

In certain embodiments, the agent capable of specifically binding to a gene product expressed on the cell surface of the immune cell is an antibody.

By means of an example, an agent, such as an antibody, capable of specifically binding to a gene product expressed on the cell surface of the immune cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo or ^{99m}Tc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

By means of an example, an agent, such as a bi-specific antibody, capable of specifically binding to a gene product expressed on the cell surface of suppressive immune cells and another cell may be used for targeting suppressive immune cells away from TILs and/or a tumor.

### Inhibitors

As used herein, the terms “inhibitor,” “antagonist,” and “silencing agent,” refer to a molecule or agent that significantly blocks, inhibits, reduces, or interferes with one or more target genes or combinations, their biological activity in vitro, in situ, and/or in vivo, including activity of downstream pathways mediated by gene signaling. In some embodiments, the inhibitor or antagonist will modulate markers of T-cell exhaustion, such as, for example, lack of/reduction in proliferation, lack of/reduction in cytokine production, lack of/reduction in cytotoxic activity, lack of/reduction in trafficking or migration, transcription factor induction, IL-10 induction, and/or elicitation of a cellular response to IL-27. Exemplary inhibitors contemplated for use in the various aspects and embodiments described herein include, but are not limited to, antibodies or antigen-binding fragments thereof that specifically bind to one or more target genes listed in Table 1 of US Pat. App. Pub. 2019/0255107, Table 10 of US Pat. App. Pub. 2019/0255107, Table 12 of US Pat. App. Pub. 2019/0255107, or Table 2 herein or gene products thereof, or one or more subunits of the target gene(s)/product(s); anti-sense molecules directed to a nucleic acid encoding the target protein or subunits thereof; short interfering RNA (“siRNA”) molecules directed to a nucleic acid encoding the target protein or subunits thereof, RNA or DNA aptamers that bind to the target gene or gene product or a subunit thereof, gene product structural analog; soluble variant proteins or fusion polypeptides thereof, DNA targeting agents, such as CRISPR systems, Zinc finger binding proteins, TALES or TALENS; and small molecule agents that target or bind to the target gene or subunit(s) thereof. In some embodiments of the compositions, methods, and uses described herein, the inhibitor inhibits some or all of IL-27 mediated signal transduction. Exemplary assays to measure inhibition or reduction of downstream IL-27 signaling pathway activities are known to those of ordinary skill in the art and/or are provided herein.

As used herein, an inhibitor or antagonist has the ability to reduce the activity and/or expression of the target gene in a cell (e.g., T cells, such as CD8+ or CD4+ T cells) by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the antagonist.

In some embodiments of the compositions, methods, and uses described herein, an inhibitor or antagonist is a monoclonal antibody.

In some embodiments of the compositions, methods, and uses described herein, an inhibitor or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen.

In some embodiments of the compositions, methods, and uses described herein, an inhibitor or antagonist is a chimeric antibody derivative of an antagonist antibody or antigen binding fragment thereof.

The inhibitor or antagonist antibodies and antigen-binding fragments thereof described herein can also be, in some embodiments, a humanized antibody derivative.

In some embodiments, the inhibitor or antagonist antibodies and antigen-binding fragments thereof described herein, i.e., antibodies that are useful for decreasing T cell exhaustion, include derivatives that are modified, i.e., by the covalent attachment of any type of molecule to the antibody, provided that the covalent attachment does not prevent the antibody from binding to the target antigen.

In some embodiments of the compositions, methods, and uses described herein, fully human antibodies are used, which are particularly desirable for the therapeutic treatment of human patients.

In some embodiments of the compositions, methods, and uses described herein, an inhibitor or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

In some embodiments of the compositions, methods, and uses described herein, an inhibitor or antagonist is an RNA or DNA aptamer that binds or physically interacts with a target gene/gene product, and blocks interactions between the gene product and a binding partner.

In some embodiments of the compositions, methods, and uses described herein, an inhibitor or antagonist comprises at least one structural analog of a target gene/gene product as listed in Table 1 of US Pat. App. Pub. 2019/0255107, Table 10 of US Pat. App. Pub. 2019/0255107, Table 12 of US Pat. App. Pub. 2019/0255107, or Table 2 herein or the combination of Prdm1 and c-Maf, or Prdm1 and c-Maf, individually. The term “structural analogs” as used herein, refers to compounds that have a similar three-dimensional structure as the target gene or portion thereof, under physiological conditions in vitro or in vivo, wherein the binding of the analog in the signaling pathway reduces a desired biological activity. Suitable structural analogs can be designed and synthesized through molecular modeling of protein binding. The structural analogs and receptor structural analogs can be monomers, dimers, or higher order multimers in any desired combination of the same or different structures to obtain improved affinities and biological effects.

In some embodiments of the compositions, methods, and uses described herein, an inhibitor or antagonist comprises at least one soluble peptide, or portion of the target gene product, or fusion polypeptide thereof. In some such embodiments, the soluble peptide is fused to an immunoglobulin constant domain, such as an Fc domain, or to another polypeptide that modifies its in vivo half-life, e.g., albumin.

In some embodiments of the compositions, methods, and uses described herein, an inhibitor or antagonist comprises at least one antisense molecule capable of blocking or decreasing the expression of a desired target gene by targeting nucleic acids encoding the gene or subunit thereof. Methods are known to those of ordinary skill in the art for the preparation of antisense oligonucleotide molecules that will specifically bind one or more target gene(s) without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, an inhibitor or antagonist comprises at least one siRNA molecule capable of blocking or decreasing the expression of a target gene product or a subunit thereof. Generally, one would prepare siRNA molecules that will specifically target one or more mRNAs without crossreacting with other polynucleotides. siRNA molecules for use in the compositions, methods, and uses described herein can be generated by methods known in the art, such as by typical solid phase oligonucleotide synthesis, and often will incorporate chemical modifications to increase half-life and/or efficacy of the siRNA agent, and/or to allow for a more robust delivery formulation. Alternatively, siRNA molecules are delivered using a vector encoding an expression cassette for intracellular transcription of siRNA.

Inhibitors or antagonists for use in the compositions, methods, and uses described herein can be identified or characterized using methods known in the art, such as protein-protein binding assays, biochemical screening assays, immunoassays, and cell-based assays, which are well known in the art.

### Activators

Also provided herein, in other aspects, are compositions comprising activators or agonists for use in the methods and compositions described herein.

As used herein, the terms “activator,” “agonist,” or “activating agent,” refer to a molecule or agent that mimics or up-regulates (e.g., increases, potentiates or supplements) the expression and/or biological activity of a target gene/gene product in vitro, in situ, and/or in vivo, including downstream pathways mediated by gene signaling. For example, in some embodiments, an activator or agonist as described herein can modulate markers of T-cell exhaustion, such as, for example, transcription factor induction (e.g., NFIL3 or T-bet induction), IL-10 induction, histone acetylation at the TIM-3 locus, TIM-3 mRNA or protein upregulation, and/or elicitation of a cellular response to IL-27. An “activator” of a given polypeptide can include the polypeptide itself, in that supplying the polypeptide itself will increase the level of the function provided by the polypeptide. An activator or agonist can be a protein or derivative thereof having at least one bioactivity of the wild-type target gene/gene product. An activator or agonist can also be a compound that up-regulates expression of the desired target gene product or its subunits. An activator or agonist can also be a compound which increases the interaction of the target gene with its receptor, for example. Exemplary activators or agonists contemplated for use in the various aspects and embodiments described herein include, but are not limited to, antibodies or antigen-binding fragments thereof that specifically bind to a target gene/gene product or subunits thereof; RNA or DNA aptamers that bind to the target gene/gene product; structural analogs or soluble mimics or fusion polypeptides thereof, DNA targeting agents, such as CRISPR systems, Zinc finger binding proteins, and TALES; and small molecule agents that target or bind to a target gene product binding partner and act as functional mimics.

As used herein, an agonist has the ability to increase or enhance the activity and/or expression of a target gene/gene product in a cell (e.g., T cells, such as CD8+ or CD4⁺ T cells) by at least 5%, at least 10%, at least 20%, at least 30%, at least 40%, at least 50%, at least 60%, at least 70%, at least 80%, at least 90%, at least 95%, at least 98%, at least 99%, at least 100%, at least 1.5-fold, at least 2-fold, at least 5-fold, at least 10-fold, at least 25-fold, at least 50-fold, at least 100-fold, at least 1000-fold, or more relative to the activity or expression level in the absence of the activator or agonist.

In some embodiments of the compositions, methods, and uses described herein, the activator or agonist increases or enhances signal transduction mediated by the target gene/gene product. In some embodiments of the compositions and methods described herein, the activator or agonist increases or enhances transcription factor induction or activation.

In some embodiments of the compositions, methods, and uses described herein, the binding sites of the activators or agonists, such as an antibody or antigen-binding fragment thereof, are directed against an interaction site between the target gene product and one or more of its binding partners. By binding to an interaction site, an activator or agonist described herein can mimic or recapitulate the binding of the target gene product to its partner and increase the activity or expression of the target gene product, and downstream signaling consequences.

In some embodiments of the compositions, methods, and uses described herein, an activator or agonist is a monoclonal antibody. In some embodiments of the compositions, methods, and uses described herein, an activator or agonist is an antibody fragment or antigen-binding fragment.

In some embodiments of the compositions, methods, and uses described herein, an activator or agonist is a chimeric antibody derivative of the agonist antibodies and antigen binding fragments thereof.

In some embodiments of the compositions, methods, and uses described herein, an activator or agonist is a humanized antibody derivative.

In some embodiments, the activator or agonist antibodies and antigen-binding fragments thereof described herein, i.e., antibodies that are useful for increasing T cell exhaustion, include derivatives that are modified, i.e., by the covalent attachment of any type of molecule to the antibody, provided that covalent attachment does not prevent the antibody from binding to the target antigen.

The activator or agonist antibodies and antigen-binding fragments thereof described herein can be generated by any suitable method known in the art.

In some embodiments, the activator or agonist antibodies and antigen-binding fragments thereof described herein are fully human antibodies or antigen-binding fragments thereof, which are particularly desirable for the therapeutic treatment of human patients. Human antibodies can be made by a variety of methods known in the art, and as described in more detail elsewhere herein.

In some embodiments of the compositions, methods, and uses described herein, an activator or agonist is a small molecule activator or agonist, including, but not limited to, small peptides or peptide-like molecules, soluble peptides, and synthetic non-peptidyl organic or inorganic compounds. A small molecule activator or agonist can have a molecular weight of any of about 100 to about 20,000 daltons (Da), about 500 to about 15,000 Da, or about 1000 to about 10,000 Da.

In some embodiments of the compositions, methods, and uses described herein, an activator or agonist is an RNA or DNA aptamer that binds or physically interacts with a target gene product and one or more of its binding partners, and enhances or promotes protein-protein interactions.

### Kits

In another aspect, the invention is directed to kit and kit of parts. The terms “kit of parts” and “kit” as used throughout this specification refer to a product containing components necessary for carrying out the specified methods (e.g., methods for detecting, quantifying or isolating immune cells as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridization probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In this description of the invention, reference is made to the accompanying drawings that form a part hereof, and in which are shown by way of illustration only of specific embodiments in which the invention may be practiced. It is to be understood that other embodiments may be utilized and structural or logical changes may be made without departing from the scope of the present invention. The description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

To facilitate an understanding of the present invention, a number of terms and phrases are defined herein:

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5% 4%, 3%, 2%, 1%, 0.5%, 0.1%, 0.05%, or 0.01% of the stated value. Unless otherwise clear from context, all numerical values provided herein are modified by the term about.

Unless specifically stated or obvious from context, as used herein, the term “or” is understood to be inclusive. Unless specifically stated or obvious from context, as used herein, the terms “a,” “an,” and “the” are understood to be singular or plural.

All gene name symbols refer to the gene as commonly known in the art. Gene symbols may be those referred to by the HUGO Gene Nomenclature Committee (HGNC). Any reference to the gene symbol is a reference made to the entire gene or variants of the gene. The HUGO Gene Nomenclature Committee is responsible for providing human gene naming guidelines and approving new, unique human gene names and symbols. All human gene names and symbols can be searched at www.genenames.org, the HGNC website, and the guidelines for their formation are available there (www.genenames.org/guidelines).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease (e.g., a neoplasia, tumor, etc.).

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a tumor specific neo-antigen polypeptide analog retains the biological activity of a corresponding naturally-occurring tumor specific neo-antigen polypeptide, while having certain biochemical modifications that enhance the analog's function relative to a naturally-occurring polypeptide. Such biochemical modifications could increase the analog's protease resistance, membrane permeability, or half-life, without altering, for example, ligand binding. An analog may include an unnatural amino acid.

“Combination therapy” is intended to embrace administration of therapeutic agents (e.g. neoantigenic peptides described herein) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of neoantigenic peptides administered at the same or different times, or they can be formulated as a single, co-formulated pharmaceutical composition comprising the peptides. As another example, a combination of the present invention (e.g., a pooled sample of tumor specific neoantigens) may be formulated as separate pharmaceutical compositions that can be administered at the same or different time. As used herein, the term “simultaneously” is meant to refer to administration of one or more agents at the same time. For example, in certain embodiments, the neoantigenic peptides are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

The term “neoantigen” or “neoantigenic” means a class of tumor antigens that arises from a tumor-specific mutation(s) which alters the amino acid sequence of genome encoded proteins.

By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “vaccine” is meant to refer in the present context to a pooled sample of tumor-specific neoantigenic peptides, for example at least two, at least three, at least four, at least five, or more neoantigenic peptides. A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination. A “vaccine composition” can include a pharmaceutically acceptable excipient, carrier or diluent.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

By an isolated “polypeptide” or “peptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The term “prime/boost” or “prime/boost dosing regimen” is meant to refer to the successive administrations of a vaccine or immunogenic or immunological compositions. The priming administration (priming) is the administration of a first vaccine or immunogenic or immunological composition type and may comprise one, two or more administrations. The boost administration is the second administration of a vaccine or immunogenic or immunological composition type and may comprise one, two or more administrations, and, for instance, may comprise or consist essentially of annual administrations. In certain embodiments, administration of the neoplasia vaccine or immunogenic composition is in a prime/boost dosing regimen. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins or RNA sequences. However, in certain embodiments, the constituent amino acids of a spacer may be selected to influence some property of the molecule such as the folding, net charge, or hydrophobicity of the molecule.

Suitable linkers for use in an embodiment of the present invention are well known to those of skill in the art and include, but are not limited to, straight or branched-chain carbon linkers, heterocyclic carbon linkers, or peptide linkers. The linker is used to separate two neoantigenic peptides by a distance sufficient to ensure that, in a preferred embodiment, each neoantigenic peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The therapy disclosed herein constitutes a new method for treating various types of cancer. The therapy described herein also provides a method of therapy for achieving clinical benefit without an unacceptable level of side effects.

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “MHC molecules”, “MHC proteins” or “HLA proteins” is thus meant proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC molecules of class II consist of an a-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the j-chain are in particular HLA-DR, HLA-DQ and HLA-DP monomers.

Subject specific HLA alleles or HLA genotype of a subject may be determined by any method known in the art. In preferred embodiments, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

In one aspect, the present disclosure provides methods for generating a prediction algorithm for identifying HLA-allele specific binding peptides, which methods comprise training a neural network with one or more peptide sequence databases (i.e, combinations of databases). In particular embodiments, the methods involve training a machine with one or more peptide sequence databases generated with a method according to the invention. More particularly, the methods comprise training a neural network running on a machine with several peptide sequence databases. In the methods provided herein, the sequences are compared so as to identify prediction algorithms for a peptide to be presented by said HLA-allele.

Generating a prediction algorithm by training a machine is a well-known technique. The most important in the training of the machine is the quality of the database used for the training. Typically, the machine combines one or more linear models, support vector machines, decision trees and/or a neural network.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory.

In a preferred embodiment, the methods involve generating models based on predictive variables. In particular embodiments, only peptide-intrinsic features are used as variables (such as sequence, amino acid properties, peptide characteristics). In alternative embodiments, the models also incorporate extrinsic features such as expression and cleavage information. In particular embodiments, the variables used to train the machine comprise one or more predictive variables selected from the group consisting of peptide sequence, amino acid physical properties, peptide physical properties, protein stability, protein translation rate, protein degradation rate, translational efficiencies from ribosomal profiling, protein cleavability, protein localization, motifs of host protein that facilitate TAP transport, whether host protein is subject to autophagy, motifs that favor ribosomal stalling (polyproline stretches) and protein features that favor NMD (long 3′ UTR, stop codon >50nt upstream of last exomexon junction). In particular embodiments, at least two of these features are used. In further embodiments, at least 3, 4, 5, 6, 7, 8, 9 or all ten of these features are used. In a preferred embodiment, the variables used to train the machine comprise the expression level of the source protein of a peptide within a cell. In a preferred embodiment, the variables used to train the machine comprise expression level of the source protein of a peptide within a cell, peptide sequence, amino acid physical properties, peptide physical properties, expression level of the source protein of a peptide within a cell, Protein stability, protein translation rate, protein degradation rate, translational efficiencies from ribosomal profiling, protein cleavability, protein localization, motifs of host protein that facilitate TAP transport, host protein is subject to autophagy, motifs that favor ribosomal stalling (polyproline stretches), protein features that favor NMD (long 3′ UTR, stop codon >50nt upstream of last exomexon junction and peptide cleavability.

In certain embodiments the present invention includes modified neoantigenic peptides. As used herein in reference to neoantigenic peptides, the terms “modified”, “modification” and the like refer to one or more changes that enhance a desired property of the neoantigenic peptide, where the change does not alter the primary amino acid sequence of the neoantigenic peptide. “Modification” includes a covalent chemical modification that does not alter the primary amino acid sequence of the neoantigenic peptide itself. Such desired properties include, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to a neoantigenic peptide that may be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R(0-CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, but certain embodiments have a molecular weight between 500 and 20,000 while other embodiments have a molecular weight between 4,000 and 10,000. The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxy succinylimide. Another activated polyethylene glycol which may be bound to a free amino group is 2,4-bis(0-methoxypolyethyleneglycol)-6-chloro-s-triazine which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties of proteins and can also be important in protein stability, secretion, and subcellular localization. Proper glycosylation can be essential for biological activity. In fact, some genes from eucaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide.

Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Dihydrofolate reductase (DHFR)—deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more polypeptides of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be affected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be affected by artificial means in other cells.

Furthermore, albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A polypeptide may also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Purification methods such as cation exchange chromatography may be used to separate conjugates by charge difference, which effectively separates conjugates into their various molecular weights. For example, the cation exchange column can be loaded and then washed with −20 mM sodium acetate, pH −4, and then eluted with a linear (0 M to 0.5 M) NaCl gradient buffered at a pH from about 3 to 5.5, e.g., at pH −4.5. The content of the fractions obtained by cation exchange chromatography may be identified by molecular weight using conventional methods, for example, mass spectroscopy, SDS-PAGE, or other known methods for separating molecular entities by molecular weight.

In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

In Vitro Peptide/Polypeptide Synthesis. Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding a neoantigenic peptide of the invention may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, Mass.).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides may be provided in the form of RNA or cDNA molecules encoding the desired neoantigenic peptides. One or more neoantigenic peptides of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neoantigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neoantigenic peptides fused in the same reading frame to create a single concatamerized neoantigenic peptide construct capable of producing multiple neoantigenic peptides.

In certain embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigenic peptide of the present invention, can be provided. By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five-point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5%>of the total number of nucleotides in the reference sequence are allowed.

The isolated tumor specific neoantigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M1 3 and filamentous single-stranded DNA phages.

Suitable host cells for expression of a polypeptide include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-ffPLC) steps employing hydrophobic RP-FTPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

In Vivo Peptide/Polypeptide Synthesis. The present invention also contemplates the use of nucleic acid molecules as vehicles for delivering neoantigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment, neoantigens may be administered to a patient in need thereof by use of a plasmid. These are plasmids which usually consist of a strong viral promoter to drive the in vivo transcription and translation of the gene (or complementary DNA) of interest (Mor, et al., (1995), The Journal of Immunology 155 (4): 2039-2046). Intron A may sometimes be included to improve mRNA stability and hence increase protein expression (Leitner et al. (1997), The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999), Adv. Parasitol. Advances in Parasitology 42: 343-410; Robinson et al., (2000). Adv. Virus Res. Advances in Virus Research 55: 1-74; Bohmet al., (1996). Journal of Immunological Methods 193 (1): 29-40). Multi cistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimising vector design for maximal protein expression is essential (Lewis et al., (1999). Advances in Virus Research (Academic Press) 54: 129-88). One way of enhancing protein expression is by optimising the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (EVI) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE DOI: 10.1371/journal.pone.Ol 18803 Apr. 13, 2015).

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more neoantigenic peptides/polypeptides as identified in according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more neoantigenic peptides of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent Application No. 20080254008, hereby incorporated by reference). The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., (1992) J. Virol. 66:2731-2739; Johann et al., (1992) J. Virol. 66: 1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment, lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via an lentivirus. Zou et al. administered about 10 μ [symbol] of a recombinant lentivirus having a titer of 1×10⁹ transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹TU/ml, more preferably at least 10⁹ TU/ml. Other methods of concentration such as ultrafiltration or binding to and elution from a matrix may be used.

In other embodiments the amount of lentivirus administered may be 1×10⁵ or about 1×10⁵ plaque forming units (PFU), 5×10⁵ or about 5×10⁵ PFU, 1×10⁶ or about 1. x10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU as total single dosage for an average human of 75 kg or adjusted for the weight and size and species of the subject. One of skill in the art can determine suitable dosage. Suitable dosages for a virus can be determined empirically. Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents of which is hereby incorporated by reference). Ad35 vectors are described in U.S. Pat. Nos. 6,974,695, 6,913,922, and 6,869,794. Adl 1 vectors are described in U.S. Pat. No. 6,913,922. C6 adenovirus vectors are described in U.S. Pat. Nos. 6,780,407; 6,537,594; 6,309,647; 6,265, 189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both E1 a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10 pu, about 4×10 pu, about 1×10 pu, about 2×10 pu, about 4×10 pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it does not integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations from about 1×10 to 1×10 genomes AAV, from about 1×10 to 1×10 genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment, effectively activating a cellular immune response for a neoplasia vaccine or immunogenic composition can be achieved by expressing the relevant neoantigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety). In another embodiment, a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 Jul.; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as: U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc. Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58: 1-13, Yilma T D. Vaccine 1989; 7: 484-485, Brochier B. Nature 1991; 354: 520-22, Wiktor, T J. Proc. Natl Acd. Sci. 1984; 81: 7194-8, Rupprecht, C E. Proc. Natl Acd. Sci. 1986; 83: 7947-50, Poulet, H Vaccine 2007; 25(Jul): 5606-12, Weyer J. Vaccine 2009; 27(November): 7198-201, Buller, R M Nature 1985; 317(6040): 813-5, Buller R M. J. Virol. 1988; 62(3):866-74, Flexner, C. Nature 1987; 330(6145): 259-62, Shida, H. J. Virol. 1988; 62(12): 4474-80, Kotwal, G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, M C. J. Virol. 2002; 76(16); 8313-34, Gallego-Gomez, J C. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, C E. J. Gen. Virol. 2007; 88: 2473-78, Mooij, P. Jour. Of Virol. 2008; 82: 2975-2988, Gomez, C E. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51: 125-30, Antonis A F. Vaccine 2007; 25: 4818-4827, B Weyer J. Vaccine 2007; 25: 4213-22, Ferrier-Rembert A. Vaccine 2008; 26(14): 1794-804, Corbett M. Proc. Natl. Acad. Sci. 2008; 105(6): 2046-51, Kaufman H L., J. Clin. Oncol. 2004; 22: 2122-32, Amato, R J. Clin. Cancer Res. 2008; 14(22): 7504-10, Dreicer R. Invest New Drugs 2009; 27(4): 379-86, Kantoff P W. J. Clin. Oncol. 2010, 28, 1099-1 105, Amato R J. J. Clin. Can. Res. 2010; 16(22): 5539-47, Kim, D W. Hum. Vaccine. 2010; 6: 784-791, Oudard, S. Cancer Immunol. Immunother. 2011; 60: 261-71, Wyatt, L S. Aids Res. Hum. Retroviruses. 2004; 20: 645-53, Gomez, C E. Virus Research 2004; 105: 11-22, Webster, D P. Proc. Natl. Acad. Sci. 2005; 102: 4836-4, Huang, X. Vaccine 2007; 25: 8874-84, Gomez, C E. Vaccine 2007a; 25: 2863-85, Esteban M. Hum. Vaccine 2009; 5: 867-871, Gomez, C E. Curr. Gene therapy 2008; 8(2): 97-120, Whelan, K T. Plos one 2009; 4(6): 5934, Scriba, T J. Eur. Jour. Immuno. 2010; 40(1): 279-90, Corbett, M. Proc. Natl. Acad. Sci. 2008; 105: 2046-2051, Midgley, C M. J. Gen. Virol. 2008; 89: 2992-97, Von Krempelhuber, A. Vaccine 2010; 28: 1209-16, Perreau, M. J. Of Virol. 2011; October: 9854-62, Pantaleo, G. Curr Opin HIV-AIDS. 2010; 5: 391-396, each of which is incorporated herein by reference.

In another embodiment, the vaccinia virus is used in the neoplasia vaccine or immunogenic composition to express a neoantigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment, ALVAC is used as a vector in a neoplasia vaccine or immunogenic composition. ALVAC is a canarypox virus that can be modified to express foreign transgenes and has been used as a method for vaccination against both prokaryotic and eukaryotic antigens (Horig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6: 2219-28; Musey L, Ding Y, Elizaga M, et al. HIV-1 vaccination administered intramuscularly can induce both systemic and mucosal T cell immunity in HIV-1-uninfected individuals. J Immunol 2003; 171: 1094-101; Paoletti E. Applications of pox virus vectors to vaccination: an update. Proc Natl Acad Sci USA 1996; 93: 11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999; 17:332-7).

In another embodiment, a Modified Vaccinia Ankara (MVA) virus may be used as a viral vector for a neoantigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In another embodiment, the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment, recombinant viral particles of the vaccine or immunogenic composition are administered to patients in need thereof. Dosages of expressed neoantigen can range from a few to a few hundred micrograms, e.g., 5 to 500 μg. The vaccine or immunogenic composition can be administered in any suitable amount to achieve expression at these dosage levels. The viral particles can be administered to a patient in need thereof or transfected into cells in an amount of about at least 10^{3 5} pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

Vaccine or Immunogenic Composition Adjuvant. Effective vaccine or immunogenic compositions advantageously include a strong adjuvant to initiate an immune response. As described herein, poly-ICLC, an agonist of TLR3 and the RNA helicase-domains of MDA5 and RIG3, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the neoantigen peptides may be combined with an adjuvant (e.g., poly-ICLC) or another antineoplastic agent. Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity, Seminars in Immunol 22: 144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans, J Exp Med 208:2357 (2011)). In a preferred embodiment Hiltonol®, a GMP preparation of poly-ICLC prepared by Oncovir, Inc, is utilized as the adjuvant. In other embodiments, other adjuvants described herein are envisioned. For instance, oil-in-water, water-in-oil or multiphasic W/O/W; see, e.g., U.S. Pat. No. 7,608,279 and Aucouturier et al, Vaccine 19 (2001), 2666-2672, and documents cited therein.

Examples of cancers and cancer conditions that can be treated with the therapy of this document include, but are not limited to, a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus, the population of subjects described herein may be suffering from one of the above cancer types. In other embodiments, the population of subjects may be all subjects suffering from solid tumors, or all subjects suffering from liquid tumors.

Of special interest is the treatment of Non-Hodgkin's Lymphoma (NHL), clear cell Renal Cell Carcinoma (ccRCC), metastatic melanoma, sarcoma, leukemia or a cancer of the bladder, colon, brain, breast, head and neck, endometrium, lung, ovary, pancreas or prostate. In certain embodiments, the melanoma is high risk melanoma.

Cancers that can be treated using the therapy described herein may include among others cases, which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

The therapy described herein is also applicable where the subject has no detectable neoplasia but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the claimed neoplastic vaccine or immunogenic composition compositions to delay disease relapse.

Pharmaceutical Compositions/Methods of Delivery. The present invention is also directed to pharmaceutical compositions comprising an effective amount of one or more neoantigenic peptides as described herein (including a pharmaceutically acceptable salt, thereof), optionally in combination with a pharmaceutically acceptable carrier, excipient or additive.

When administered as a combination, the therapeutic agents (i.e. the neoantigenic peptides) can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

Surgical resection uses surgery to remove abnormal tissue in cancer, such as mediastinal, neurogenic, or germ cell tumors, or thymoma. In certain embodiments, administration of the composition is initiated following tumor resection. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. Preferably, administration of the neoplasia vaccine or immunogenic composition is initiated 4, 5, 6, 7, 8, 9, 10, 11 or 12 weeks after tumor resection.

Prime/boost regimens refer to the successive administrations of a vaccine or immunogenic or immunological compositions. In certain embodiments, administration of the neoplasia vaccine or immunogenic composition is in a prime/boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to elicit a greater cytotoxic T-cell response (see Schneider et al., Induction of CD8+ T cells using heterologous prime-boost immunisation strategies, Immunological Reviews Volume 170, Issue 1, pages 29-38, August 1999). In another embodiment, DNA encoding neoantigens is used to prime followed by a protein boost. In another embodiment, protein is used to prime followed by boosting with a virus encoding the neoantigen. In another embodiment, a virus encoding the neoantigen is used to prime and another virus is used to boost. In another embodiment, protein is used to prime and DNA is used to boost. In a preferred embodiment a DNA vaccine or immunogenic composition is used to prime a T-cell response and a recombinant viral vaccine or immunogenic composition is used to boost the response. In another preferred embodiment, a viral vaccine or immunogenic composition is coadministered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun. 2007 December; 75(12):5819-26. Epub 2007 Oct. 1). The pharmaceutical compositions can be processed in accordance with conventional methods of pharmacy to produce medicinal agents for administration to patients in need thereof, including humans and other mammals.

Modifications of the neoantigenic peptides can affect the solubility, bioavailability and rate of metabolism of the peptides, thus providing control over the delivery of the active species. Solubility can be assessed by preparing the neoantigenic peptide and testing according to known methods well within the routine practitioner's skill in the art.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable carrier further comprises dimethylsulfoxide. In certain embodiments, the pharmaceutical composition further comprises an immunomodulator or adjuvant. In certain embodiments, the immunodulator or adjuvant is selected from the group consisting of poly-ICLC, STING agonist, 1018 ISS, aluminum salts, Amplivax, AS15, BCG, CP-870,893, CpG7909, CyaA, dSLEVI, GM-CSF, IC30, IC31, Imiquimod, ImuFact FMP321, IS Patch, ISS, ISCOMATRLX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8: 1396-1401). The vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety).

In certain embodiments, the pH modifier can stabilize the adjuvant or immunomodulator as described herein.

In certain embodiments, a pharmaceutical composition comprises: one to five peptides, dimethylsulfoxide (DMSO), dextrose, water, succinate, poly I: poly C, poly-L-lysine, carboxymethylcellulose, and chloride. In certain embodiments, each of the one to five peptides is present at a concentration of 300 μg/ml. In certain embodiments, the pharmaceutical composition comprises <3% DMSO by volume. In certain embodiments, the pharmaceutical composition comprises 3.6-3.7% dextrose in water. In certain embodiments, the pharmaceutical composition comprises 3.6-3.7 mM succinate (e.g., as sodium succinate) or a salt thereof. In certain embodiments, the pharmaceutical composition comprises 0.5 mg/ml poly I: poly C. In certain embodiments, the pharmaceutical composition comprises 0.375 mg/ml poly-L-Lysine. In certain embodiments, the pharmaceutical composition comprises 1.25 mg/ml sodium carboxymethylcellulose. In certain embodiments, the pharmaceutical composition comprises 0.225% sodium chloride.

Pharmaceutical compositions comprise the herein-described tumor specific neoantigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials which modify the physical form of the dosage unit, for example, coatings of sugar, shellac, or enteric agents. Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed. Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, subcutaneous, transdermal (which may include a penetration enhancement agent), buccal and suppository administration, among other routes of administration, including through an eye or ocular route.

The neoplasia vaccine or immunogenic composition, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the vaccine or immunogenic composition is administered intravenously or subcutaneously. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific neoantigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neoantigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal antiinflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as HERCEPTIN and RITUXAN.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P.G.M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The additional agents that may be included with the tumor specific neo-antigenic peptides of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Dosage. When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with neoplasia, e.g. cancer or tumors.

A preferred dose of an agent is the maximum that a patient can tolerate and not develop serious or unacceptable side effects. Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 mg/ml. The pharmaceutical compositions 5 typically should provide a dosage of from about 0.001 mg to about 2000 mg of compound per kilogram of body weight per day. For example, dosages for systemic administration to a human patient can range from 1-10 mg/kg, 20-80 mg/kg, 5-50 mg/kg, 75-150 mg/kg, 100-500 mg/kg, 250-750 mg/kg, 500-1000 mg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 10 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about IμM of an agent is administered to a subject. In related embodiments, about 50-100 nM, 50-250 nM, 100-500 nM, 250-500 nM, 250-750 nM, 500-750 nM, 500 nM to IμM. or 750 nM to IμM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Preferred unit dosage formulations are those containing a daily dose or unit, daily sub-dose, as herein discussed, or an appropriate fraction thereof, of the administered ingredient.

The dosage regimen for treating a disorder or a disease with the tumor specific neoantigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art.

The amount of compound included within therapeutically active formulations according to the present invention is an effective amount for treating the disease or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

For oral administration to humans, a dosage of between approximately 0.1 to 100 mg/kg/day, preferably between approximately 1 and 100 mg/kg/day, is generally sufficient.

Where drug delivery is systemic rather than topical, this dosage range generally produces effective blood level concentrations of active compound ranging from less than about 0.04 to about 400 micrograms/cc or more of blood in the patient. The compound is conveniently administered in any suitable unit dosage form, including but not limited to one containing 0.001 to 3000 mg, preferably 0.05 to 500 mg of active ingredient per unit dosage form. An oral dosage of 10-250 mg is usually convenient.

According to certain exemplary embodiments, the vaccine or immunogenic composition is administered at a dose of about 10 μg to 1 mg per neoantigenic peptide. According to certain exemplary embodiments, the vaccine or immunogenic composition is administered at an average weekly dose level of about 10 μg to 2000 μg per neoantigenic peptide.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The invention provides for pharmaceutical compositions containing at least one tumor specific neoantigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabi sulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container. Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hy droxy ethyl-L-glutamine) and poly(lactic acid). Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects. Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZO E.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone. In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents. Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

Vaccine or immunogenic compositions. The present invention is directed in some aspects to pharmaceutical compositions suitable for the prevention or treatment of cancer. In one embodiment, the composition comprises at least an immunogenic composition, e.g., a neoplasia vaccine or immunogenic composition capable of raising a specific T-cell response. The neoplasia vaccine or immunogenic composition comprises neoantigenic peptides and/or neoantigenic polypeptides corresponding to tumor specific neoantigens as described herein.

A suitable neoplasia vaccine or immunogenic composition can preferably contain a plurality of tumor specific neoantigenic peptides. In an embodiment, the vaccine or immunogenic composition can include between 1 and 100 sets of peptides, more preferably between 1 and 50 such peptides, even more preferably between 10 and 30 sets peptides, even more preferably between 15 and 25 peptides. According to another preferred embodiment, the vaccine or immunogenic composition can include at least one peptides, more preferably 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides.

The optimum amount of each peptide to be included in the vaccine or immunogenic composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 μg and 1.5 mg, preferably g to 500 μg, of peptide or DNA may be given and can depend from the respective peptide or DNA. Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine or immunogenic composition are known to those skilled in the art.

In one embodiment of the present invention the different tumor specific neoantigenic peptides and/or polypeptides are selected for use in the neoplasia vaccine or immunogenic composition so as to maximize the likelihood of generating an immune attack against the neoplasias/tumors in a high proportion of subjects in the population. Without being bound by theory, it is believed that the inclusion of a diversity of tumor specific neoantigenic peptides can generate a broad scale immune attack against a neoplasia/tumor. In one embodiment, the selected tumor specific neoantigenic peptides/polypeptides are encoded by missense mutations. In a second embodiment, the selected tumor specific neoantigenic peptides/polypeptides are encoded by a combination of missense mutations and neoORF mutations. In a third embodiment, the selected tumor specific neoantigenic peptides/polypeptides are encoded by neoORF mutations.

In one embodiment in which the selected tumor specific neoantigenic peptides/polypeptides are encoded by missense mutations, the peptides and/or polypeptides are chosen based on their capability to associate with the MHC molecules of a high proportion of subjects in the population. Peptides/polypeptides derived from neoOR mutations can also be selected on the basis of their capability to associate with the MHC molecules of the patient population.

The vaccine or immunogenic composition is capable of raising a specific cytotoxic T-cells response and/or a specific helper T-cell response.

The vaccine or immunogenic composition can further comprise an adjuvant and/or a carrier. Examples of useful adjuvants and carriers are given herein. The peptides and/or polypeptides in the composition can be associated with a carrier such as, e.g., a protein or an antigen-presenting cell such as e.g. a dendritic cell (DC) capable of presenting the peptide to a T-cell. Adjuvants are any substance whose admixture into the vaccine or immunogenic composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLEVI, GM-CSF, IC30, IC31, Imiquimod, ImuFact FMP321, IS Patch, ISS, ISCOMATRIX, Juvlmmune, LipoVac, MF59, monophosphoryl lipid A, Montanide FMS 1312, Montanide ISA 206, Montanide ISA 50V, Montanide ISA-51, OK-432, OM-174, OM-197-MP-EC, ONTAK, PEPTEL. vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasi s Tumor Immunol. 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co-stimulatory molecules in DCsl. Furthermore, poly-ICLC was recently directly compared to CpG in non-human primates (rhesus macaques) as adjuvant for a protein vaccine or immunogenic composition consisting of human papillomavirus (HPV)16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4)).

CpG immuno stimulatory oligonucleotides have also been reported to enhance the effects of adjuvants in a vaccine or immunogenic composition setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IF A) that normally promote a Th2 bias. CpG oligonucleotides show even greater adjuvant activity when formulated or coadministered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLEVI (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the Pl/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8+ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD8+ T-cell and antibody responses were markedly enhanced with the addition of poly-ICLC and Montanide compared to OLP alone or OLP and Montanide.

A vaccine or immunogenic composition according to the present invention may comprise more than one different adjuvant. Furthermore, the invention encompasses a therapeutic composition comprising any adjuvant substance including any of those herein discussed. It is also contemplated that the peptide or polypeptide, and the adjuvant can be administered separately in any appropriate sequence. A carrier may be present independently of an adjuvant. The carrier may be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diptheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine or immunogenic composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule may readily be loaded with the selected antigen in vitro.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient's tumor. CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type 1 (TH1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells non-specifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD8+ cells; 2) an intermediate level in which native, tumor-associated CD4+ epitopes may be used to augment CD8+ cells; and 3) a patient specific level in which neoantigen CD4+ epitopes may be used to augment CD8+ cells in a patient specific manner. Although current algorithms for predicting CD4 epitopes are limited in accuracy, it is a reasonable expectation that many long peptides containing predicted CD8 neoepitopes will also include CD4 epitopes. CD4 epitopes are longer than CD8 epitopes and typically are 10-12 amino acids in length although some can be longer (Kreiter et al, Mutant MHIC Class II epitopes drive therapeutic immune responses to cancer, Nature (2015). Thus the neoanti genie epitopes described herein, either in the form of long peptides (>25 amino acids) or nucleic acids encoding such long peptides, may also boost CD4 responses in a tumor and patient-specific manner (level (3) above).

CD8+ cell immunity may also be generated with neoantigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gplOO melanoma antigen-derived peptides with autologous peptide pulsed CD40L/IFN-g-activated mature DCs via an IL-12p70-producing patient DC vaccine (see e.g., Carreno et al (2013) L-12p70-producing patient DC vaccine elicits Tel-polarized immunity, Journal of Clinical Investigation, 123(8):3383-94 and Ali et al. (2009) In situ regulation of DC subsets and T cells mediates tumor regression in mice, Cancer Immunotherapy, 1(8): 1-10). It is contemplated within the scope of the invention that neoantigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-12), tumor necrosis factor (TNF), interferon gamma-induced protein 10 (IP-10), interleukin 1 (IL-1), and type I interferons (IFN), and minimal interleukin 10 (IL-10) production. DCs may be differentiated from frozen peripheral blood mononuclear cells (PBMCs) obtained by leukapheresis, while PBMCs may be isolated by Ficoll gradient centrifugation and frozen in aliquots.

Illustratively, the following 7 day activation protocol may be used. Day 1 PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. Aug. 1; (78). doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact with T cells to generate a potent cytotoxic T lymphocyte response to the cancer neoantigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1(8): 1-10; Ali et al. (2009) Infection-mimicking materials to program dendritic cells in situ. Nat Mater 8: 151-8), and such a vaccine is currently being tested in a Phase I clinical trial recently initiated at the Dana-Farber Cancer Institute. This approach has also been shown to lead to regression of glioblastoma, as well as the induction of a potent memory response to prevent relapse, using the C6 rat glioma model.24 in the current proposal. The ability of such an implantable, biomatrix vaccine delivery scaffold to amplify and sustain tumor specific dendritic cell activation may lead to more robust anti-tumor immunosensitization than can be achieved by traditional subcutaneous or intra-nodal vaccine administrations.

The present invention may include any method for loading a neoantigenic peptide onto a dendritic cell. One such method applicable to the present invention is a microfluidic intracellular delivery system. Such systems cause temporary membrane disruption by rapid mechanical deformation of human and mouse immune cells, thus allowing the intracellular delivery of biomolecules (Sharei et al., 2015, PLOS ONE).

Preferably, the antigen presenting cells are dendritic cells. Suitably, the dendritic cells are autologous dendritic cells that are pulsed with the neoantigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. In certain embodiments the dendritic cells are targeted using CD141, DEC205, or XCR1 markers. CD141+XCR1+ DCs were identified as a subset that may be better suited to the induction of anti-tumor responses (Bachem et al., J. Exp. Med. 207, 1273-1281 (2010); Crozat et al., J. Exp. Med. 207, 1283-1292 (2010); and Gallois & Bhardwaj, Nature Med. 16, 854-856 (2010)).

Thus, in one embodiment of the present invention the vaccine or immunogenic composition containing at least one antigen presenting cell is pulsed or loaded with one or more peptides of the present invention. Alternatively, peripheral blood mononuclear cells (PBMCs) isolated from a patient may be loaded with peptides ex vivo and injected back into the patient. As an alternative the antigen presenting cell comprises an expression construct encoding a peptide of the present invention. The polynucleotide may be any suitable polynucleotide and it is preferred that it is capable of transducing the dendritic cell, thus resulting in the presentation of a peptide and induction of immunity.

The inventive pharmaceutical composition may be compiled so that the selection, number and/or amount of peptides present in the composition covers a high proportion of subjects in the population. The selection may be dependent on the specific type of cancer, the status of the disease, earlier treatment regimens, and, of course, the HLA-haplotypes present in the patient population.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life-threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine or immunogenic compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant can, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more neoantigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter-see documents cited or incorporated herein for useful promoters), DNA for a eukaryotic leader peptide for secretion (e.g., tissue plasminogen activator), DNA for the neoantigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different neoantigen. Mention is also made of Wasmoen U.S. Pat. No. 5,849,303, and Dale U.S. Pat. No. 5,811,104, whose text may be useful. DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 may be relied upon for DNA plasmid teachings that can be employed in constructing and using DNA plasmids that contain and express in vivo.

The nucleic acids can also be delivered complexed to cationic compounds, such as cationic lipids. Lipid-mediated gene delivery methods are described, for instance, in W01996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al, 2011; see also U.S. Pat. No. 8,278,036; Halabi et al. J Clin Oncol (2003) 21: 1232-1237; Petsch et al, Nature Biotechnology 2012 Dec. 7; 30(12): 1210-6).

Viral vectors as described herein can also be used to deliver the neoantigenic peptides of the invention. Vectors can be administered so as to have in vivo expression and response akin to doses and/or responses elicited by antigen administration.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted herein, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. FM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that allows about 200, and preferably 200 or more, human Class I MHC molecules loaded with peptide to be expressed on the surface of each stimulator cell. Preferably, the stimulator cells are incubated with >2 g/ml peptide. For example, the stimulator cells are incubates with >3, 4, 5, 10, 15, or more g/ml peptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+(effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte: stimulator cell ratio is in the range of about 30:1 to 300:1. The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses.

Since mutant cell lines do not exist for every human MHC allele, it is advantageous to use a technique to remove endogenous MHC-associated peptides from the surface of APC, followed by loading the resulting empty MHC molecules with the immunogenic peptides of interest. The use of non-transformed (non-tumorigenic), noninfected cells, and preferably, autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its a1 and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destabilize p2microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions do not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody-tagged molecules may then be extracted from the stimulator-effector cell admixture via appropriate means, e.g., via well-known immunoprecipitation or immunoassay methods.

Effective, cytotoxic amounts of the activated CD8+ cells can vary between in vitro and in vivo uses, as well as with the amount and type of cells that are the ultimate target of these killer cells. The amount can also vary depending on the condition of the patient and should be determined via consideration of all appropriate factors by the practitioner. Preferably, however, about 1×10⁶ to about 1×10¹², more preferably about 1×10⁸ to about 1×10¹¹, and even more preferably, about 1×10⁹ to about 1×10¹⁰ activated CD8+ cells are utilized for adult humans, compared to about 5×10⁶-5×10⁷ cells used in mice.

Preferably, as discussed herein, the activated CD 8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments are discussed in the sections that follow.

Therapeutic Methods. The present invention provides methods of inducing a neoplasia/tumor specific immune response in a subject, vaccinating against a neoplasia/tumor, treating and or alleviating a symptom of cancer in a subject by administering the subject a plurality of neoantigenic peptides or composition of the invention. According to the invention, the herein-described neoplasia vaccine or immunogenic composition may be used for a patient that has been diagnosed as having cancer, or at risk of developing cancer.

The claimed combination of the invention is administered in an amount sufficient to induce a CTL response.

Additional Therapies. The tumor specific neoantigen peptides and pharmaceutical compositions described herein can also be administered in a combination therapy with another agent, for example a therapeutic agent. In certain embodiments, the additional agents can be, but are not limited to, chemotherapeutic agents, anti-angiogenesis agents and agents that reduce immune-suppression.

The neoplasia vaccine or immunogenic composition can be administered before, during, or after administration of the additional agent. In embodiments, the neoplasia vaccine or immunogenic composition is administered before the first administration of the additional agent. In other embodiments, the neoplasia vaccine or immunogenic composition is administered after the first administration of the additional therapeutic agent (e.g., 1, 2, 3, 4, 5, 6, 7, 8, 9, 10, 11, 12, 13, 14 days or more). In embodiments, the neoplasia vaccine or immunogenic composition is administered simultaneously with the first administration of the additional therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin Kl-3, DL-a-Difluorom ethyl-ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-di oxide, Aminopterin, Cytosine 3-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole I-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-a, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DTIC), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody or small molecule such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tositumomab, ado-trastuzumab emtansine, lapatinib (Tykerb®), pertuzumab (Perjeta™), ado-trastuzumab emtansine (Kadcyla™), regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatinib dimaleate (Gilotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), pembrolizumab (Keytruda®), carfilzomib (Kyprolis®), Ramucirumab (Cyramza™), Cabozantinib (Cometriq™), vandetanib (Caprelsa®), Optionally, the therapeutic agent is a neoantigen. The therapeutic agent may be a cytokine such as interferons (INFs), interleukins (ILs), or hematopoietic growth factors. The therapeutic agent may be INF-a, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vonnostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). Additionally, the therapeutic agent may be an epigenetic targeted drug such as FIDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi). For prostate cancer treatment, a preferred chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al. Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622. 2004). The observation that GITR stimulation has immunostimulatory effects in vitro and induced autoimmunity in vivo prompted the investigation of the antitumor potency of triggering this pathway. A review of Modulation Of Ctla 4 And Gitr For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG3, ICOS, PDL1 or KIR (Page et a, Annual Review of Medicine 65:27 (2014)). In further additional embodiments, the checkpoint inhibitor is targeted at a member of the TNFR superfamily such as CD40, OX40, CD 137, GITR, CD27 or TEVI-3. In some cases targeting a checkpoint inhibitor is accomplished with an inhibitory antibody or similar molecule. In other cases, it is accomplished with an agonist for the target; examples of this class include the stimulatory targets OX40 and GITR.

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 January; 8(1):59-73). Additionally, chemotherapeutic agents can be administered safely with immunotherapy without inhibiting vaccine specific T-cell responses (Perez et al., A new era in anticancer peptide vaccines. Cancer May 2010). In one embodiment the additional agent is administered to increase the efficacy of the therapy described herein. In one embodiment the additional agent is a chemotherapy treatment. In one embodiment low doses of chemotherapy potentiate delayed-type hypersensitivity (DTH) responses. In one embodiment the chemotherapy agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al., Multipeptide immune response to cancer vaccine IMA901 after single-dose cyclophosphamide associates with longer patient survival. Nature Medicine; 18:8 2012). In another embodiment, cyclophosphamide is administered according to a metronomic program, where a daily dose is administered for one month (Ghiringhelli et al., Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T-cell and NK-cell functions (Zitvogel et al., 2008, Nat. Rev. Immunol., 8(1):59-73). In another embodiment a low dose of a chemotherapeutic agent is administered with the therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination was combined with a low dose of estramustine (Itoh et al., Personalized peptide vaccines: A new therapeutic modality for cancer. Cancer Sci 2006; 97: 970-976). In another embodiment glucocorticoids are administered with or before the therapy described herein (Zitvogel et al., 2008, Nat. Rev. Immunol., 8(1):59-73). In another embodiment glucocorticoids are administered after the therapy described herein. In another embodiment Gemcitabine is administered before, simultaneously, or after the therapy described herein to enhance the frequency of tumor specific CTL precursors (Zitvogel et al., 2008, Nat. Rev. Immunol., 8(1):59-73). In another embodiment 5-fluorouracil is administered with the therapy described herein as synergistic effects were seen with a peptide-based vaccine (Zitvogel et al., 2008, Nat. Rev. Immunol., 8(1):59-73). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al., BRAF inhibition is associated with enhanced melanoma antigen expression and a more favorable tumor microenvironment in patients with metastatic melanoma. Clin Cancer Res. 2013; 19: 1225-1231). In another embodiment an inhibitor of tyrosine kinases is used as an additional agent. In one embodiment the tyrosine kinase inhibitor is used before vaccination with the therapy described herein. In one embodiment the tyrosine kinase inhibitor is used simultaneously with the therapy described herein. In another embodiment the tyrosine kinase inhibitor is used to create a more immune permissive environment. In another embodiment the tyrosine kinase inhibitor is sunitinib or imatinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the therapy described herein. Doses of targeted therapies has been described previously (Alvarez, Present and future evolution of advanced breast cancer therapy. Breast Cancer Research 2010, 12(Suppl 2):S1). In another embodiment temozolomide is administered with the therapy described herein. In one embodiment temozolomide is administered at 200 mg/day for 5 days every fourth week of a combination therapy with the therapy described herein. Results of a similar strategy have been shown to have low toxicity (Kyte et al., Tel om erase Peptide Vaccination Combined with Temozolomide: A Clinical Trial in Stage IV Melanoma Patients. Clin Cancer Res; 17(13) 2011). In another embodiment the therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

Patients in need thereof may receive a series of priming vaccinations with a mixture of tumor-specific peptides. Additionally, over a 4-week period the priming may be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients may be enrolled. Peripheral blood is extensively monitored for peptide-specific T-cell responses and patients are followed for up to two years to assess disease recurrence.

Administering a combination therapy consistent with standard of care. In another aspect, the therapy described herein provides selecting the appropriate point to administer a combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (www.cancer.gov/cancertopics). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The combination therapy described herein can be incorporated within the standard of care. The combination therapy described herein may also be administered where the standard of care has changed due to advances in medicine.

Incorporation of the combination therapy described herein may depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of T F, and taxanes (Zitvogel et al., 2008, Nat. Rev. Immunol., 8(1):59-73). The combination therapy may be administered before such steps or may be administered after.

In one embodiment the combination therapy may be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high-dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also may be used. In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient's body after the chemotherapy and/or radiation therapy (the tumor) as foreign and attack them. Immunotherapy with the combination therapy described herein can take advantage of this by vaccinating after a transplant. Additionally, the transferred cells may be presented with neoantigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells may break away from the primary tumor and spread to other parts of the body (metastasize). Therefore, doctors give adjuvant therapy to kill any cancer cells that may have spread, even if they cannot be detected by imaging or laboratory tests.

In one embodiment the combination therapy is administered consistent with the standard of care for Ductal carcinoma in situ (DCIS). The standard of care for this breast cancer type is: 1. Breast-conserving surgery and radiation therapy with or without tamoxifen; 2. Total mastectomy with or without tamoxifen; 3. Breast-conserving surgery without radiation therapy. The combination therapy may be administered before breast conserving surgery or total mastectomy to shrink the tumor before surgery. In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. The standard of care for this breast cancer type is: 1. Local-regional treatment: Breast-conserving therapy (lumpectomy, breast radiation, and surgical staging of the axilla), Modified radical mastectomy (removal of the entire breast with level I-II axillary dissection) with or without breast reconstruction, Sentinel node biopsy. 2. Adjuvant radiation therapy postmastectomy in axillary node-positive tumors: For one to three nodes: unclear role for regional radiation (infra/supraclavicular nodes, internal mammary nodes, axillary nodes, and chest wall). For more than four nodes or extranodal involvement: regional radiation is advised. 3. Adjuvant systemic therapy. In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is: 1. Multimodality therapy delivered with curative intent is the standard of care for patients with clinical stage IIIB disease. 2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy may consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy may consist of further chemotherapy. Hormone therapy should be administered to patients whose tumors are ER-positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zitvogel et al., 2008, Nat. Rev. Immunol., 8(1):59-73). Treatment after administering the combination therapy may negatively affect dividing effector T-cells. The combination therapy may also be administered after radiation.

In another embodiment the combination therapy described herein is used in the treatment in a cancer where the standard of care is primarily not surgery and is primarily based on systemic treatments, such as Chronic Lymphocytic Leukemia (CLL).

In another embodiment patients diagnosed with stage I, II, III, and IV Chronic Lymphocytic Leukemia are treated with the combination therapy as described herein. The standard of care for this cancer type is: 1. Observation in asymptomatic or minimally affected patients, 2. Rituximab, 3. Ofatumomab, 4. Oral alkylating agents with or without corticosteroids, 5. Fludarabine, 2-chlorodeoxyadenosine, or pentostatin, 6. Bendamustine, 7. Lenalidomide and 8. Combination chemotherapy. Combination chemotherapy regimens include the following: Fludarabine plus cyclophosphamide plus rituximab. o Fludarabine plus rituximab as seen in the CLB-9712 and CLB-9011 trials, o Fludarabine plus cyclophosphamide versus fludarabine plus cyclophosphamide plus rituximab, Pentostatin plus cyclophosphamide plus rituximab as seen in the MAYO-MC0183 trial, for example, Ofatumumab plus fludarabine plus cyclophosphamide, CVP: cyclophosphamide plus vincristine plus prednisone, CHOP: cyclophosphamide plus doxorubicin plus vincristine plus prednisone, Fludarabine plus cyclophosphamide versus fludarabine as seen in the E2997 trial [NCT00003764] and the LRF-CLL4 trial, for example, Fludarabine plus chlorambucil as seen in the CLB-9011 trial, for example. 9. Involved-field radiation therapy. 10. Alemtuzumab 11. Bone marrow and peripheral stem cell transplantations are under clinical evaluation. 12. Ibrutinib

In one embodiment the combination therapy is administered before, simultaneously with or after treatment with Rituximab or Ofatumomab. As these are monoclonal antibodies that target B-cells, treatment with the combination therapy may be synergistic. In another embodiment the combination therapy is administered after treatment with oral alkylating agents with or without corticosteroids, and Fludarabine, 2-chlorodeoxyadenosine, or pentostatin, as these treatments may negatively affect the immune system if administered before. In one embodiment bendamustine is administered with the combination therapy in low doses based on the results for prostate cancer described herein. In one embodiment the combination therapy is administered after treatment with bendamustine.

In another embodiment, therapies targeted to specific recurrent mutations in genes that include extracellular domains are used in the treatment of a patient in need thereof suffering from cancer. The genes may advantageously be well-expressed genes. Well expressed may be expressed in “transcripts per million” (TPM). A TPM greater than 100 is considered well expressed. Well expressed genes may be FGFR3, ERBB3, EGFR, MUC4, PDGFRA, MMP12, TMEM52, and PODXL. The therapies may be a ligand capable of binding to an extracellular neoantigen epitope. Such ligands are well known in the art and may include therapeutic antibodies or fragments thereof, antibody-drug conjugates, engineered T cells, or aptamers. Engineered T cells may be chimeric antigen receptors (CARs). Antibodies may be fully humanized, humanized, or chimeric. The antibody fragments may be a nanobody, Fab, Fab′, (Fab′)2, Fv, ScFv, diabody, triabody, tetrabody, Bis-scFv, minibody, Fab2, or Fab3 fragment. Antibodies may be developed against tumor-specific neoepitopes using known methods in the art.

Adoptive cell transfer (ACT). Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912, 170; 6,004,811; 6,284,240; 6,392,013; 6,410,014; 6,753,162; 8,211,422; and, PCT Publication WO9215322). Alternative CAR constructs may be characterized as belonging to successive generations. First-generation CARs typically consist of a single-chain variable fragment of an antibody specific for an antigen, for example comprising a V_{L} linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction).

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365: 1673-1683; Sadelain M, The New England Journal of Medicine 2011; 365: 1735-173; Ramos et al., Stem Cells 28(6): 1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf adoptive T-cell immunotherapies, Cancer Res 75 (18): 3853). Cells may be edited using any DNA targeting protein, including, but not limited to a CRISPR system, Zinc Finger binding protein, TALE or TALEN as known in the art. DNA targeting proteins may be delivered to an immune cell by any method known in the art. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, a and β, which assemble to form a heterodimer and associates with the CD3-transducing subunits to form the T cell receptor complex present on the cell surface. Each a and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRa or TCRP can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor a-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD 137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD 160, TIGIT, CD96, CRT AM, LAIR1, SIGLEC7, SIGLEC9, CD244 (2B4), T FRSF10B, TNFRSF10A, CASP8, C ASP 10, CASP3, CASP6, CASP7, FADD, FAS, TGFBRII, TGFRBRI, SMAD2, SMAD3, SMAD4, SMADIO, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD 137, GITR, CD27, SHP-1 or TEVI-3. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRa, PD1 and TCRp, CTLA-4 and TCRa, CTLA-4 and TCRp, LAG3 and TCRa, LAG3 and TCRp, Tim3 and TCRa, Tim3 and TCRp, BTLA and TCRa, BTLA and TCRp, BY55 and TCRa, BY55 and TCRp, TIGIT and TCRa, TIGIT and TCRp, B7H5 and TCRa, B7H5 and TCRp, LAIR1 and TCRa, LAIR1 and TCRp, SIGLEC10 and TCRa, SIGLEC10 and TCRp, 2B4 and TCRa, 2B4 and TCRp.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7, 175,843; 5,883,223; 6,905,874; 6,797,514; 6,867,041; and 7,572,631. T cells can be expanded in vitro or in vivo.

Vaccine or Immunogenic Composition Kits and Co-Packaging. In an aspect, the invention provides kits containing any one or more of the elements discussed herein to allow administration of the therapy. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously allow the provision of all elements of the systems of the invention. Kits can involve vector(s) and/or particle(s) and/or nanoparticle(s) containing or encoding RNA(s) for 1-50 or more neoantigen mutations to be administered to an animal, mammal, primate, rodent, etc., with such a kit including instructions for administering to such a eukaryote; and such a kit can optionally include any of the anti-cancer agents described herein. The kit may include any of the components above (e.g. vector(s) and/or particle(s) and/or nanoparticle(s) containing or encoding RNA(s) for 1-50 or more neoantigen mutations, neoantigen proteins or peptides) as well as instructions for use with any of the methods of the present invention. In one embodiment the kit contains at least one vial with an immunogenic composition or vaccine. In one embodiment the kit contains at least one vial with an immunogenic composition or vaccine and at least one vial with an anticancer agent. In one embodiment kits may comprise ready to use components that are mixed and ready to administer. In one aspect a kit contains a ready to use immunogenic or vaccine composition and a ready to use anti-cancer agent. The ready to use immunogenic or vaccine composition may comprise separate vials containing different pools of immunogenic compositions. The immunogenic compositions may comprise one vial containing a viral vector or DNA plasmid and the other vial may comprise immunogenic protein. The ready to use anticancer agent may comprise a cocktail of anticancer agents or a single anticancer agent. Separate vials may contain different anticancer agents. In another embodiment a kit may contain a ready to use anti-cancer agent and an immunogenic composition or vaccine in a ready to be reconstituted form. The immunogenic or vaccine composition may be freeze dried or lyophilized. The kit may comprise a separate vial with a reconstitution buffer that can be added to the lyophilized composition so that it is ready to administer. The buffer may advantageously comprise an adjuvant or emulsion according to the present invention. In another embodiment the kit may comprise a ready to reconstitute anticancer agent and a ready to reconstitute immunogenic composition or vaccine. In this aspect both may be lyophilized. In this aspect separate reconstitution buffers for each may be included in the kit. The buffer may advantageously comprise an adjuvant or emulsion according to the present invention. In another embodiment the kit may comprise single vials containing a dose of immunogenic composition and anti-cancer agent that are administered together. In another aspect multiple vials are included so that one vial is administered according to a treatment timeline. One vial may only contain the anti-cancer agent for one dose of treatment, another may contain both the anti-cancer agent and immunogenic composition for another dose of treatment, and one vial may only contain the immunogenic composition for yet another dose. In a further aspect the vials are labeled for their proper administration to a patient in need thereof. The immunogen or anti-cancer agents of any embodiment may be in a lyophilized form, a dried form or in aqueous solution as described herein. The immunogen may be a live attenuated virus, protein, or nucleic acid as described herein.

In one embodiment the anticancer agent is one that enhances the immune system to enhance the effectiveness of the immunogenic composition or vaccine. In a preferred embodiment the anti-cancer agent is a checkpoint inhibitor. In another embodiment the kit contains multiple vials of immunogenic compositions and anti-cancer agents to be administered at different time intervals along a treatment plan. In another embodiment the kit may comprise separate vials for an immunogenic composition for use in priming an immune response and another immunogenic composition to be used for boosting. In one aspect the priming immunogenic composition could be DNA or a viral vector and the boosting immunogenic composition may be protein. Either composition may be lyophilized or ready for administering. In another embodiment different cocktails of anti-cancer agents containing at least one anticancer agent are included in different vials for administration in a treatment plan.

Although the present invention and its advantages have been described in detail, it should be understood that various changes, substitutions and alterations can be made herein without departing from the spirit and scope of the invention as defined in the appended claims.

Further embodiments are illustrated in the following Examples which are given for illustrative purposes only and are not intended to limit the scope of the invention.

### EXAMPLES

### Example 1—a Transcriptional Atlas of Different CAR T Cells

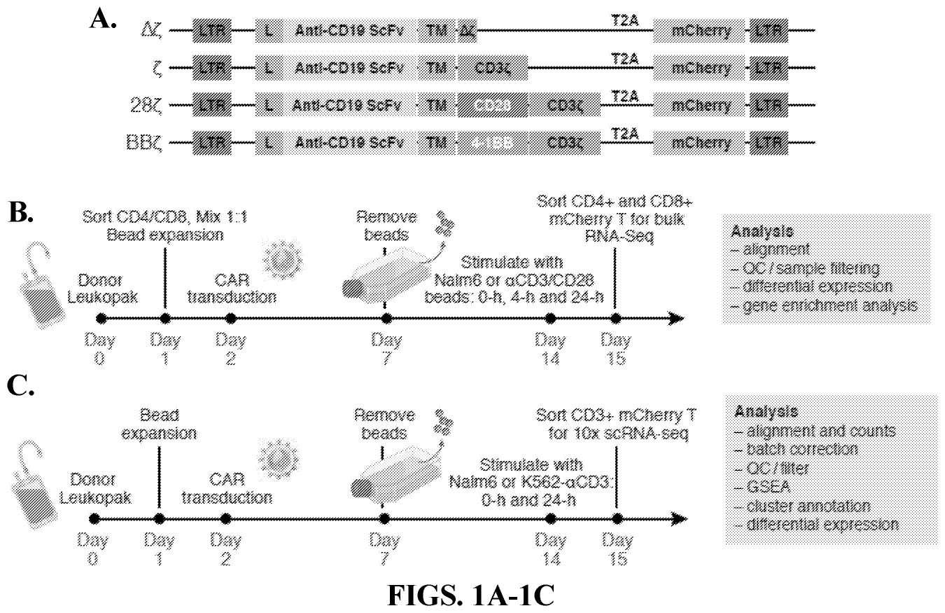

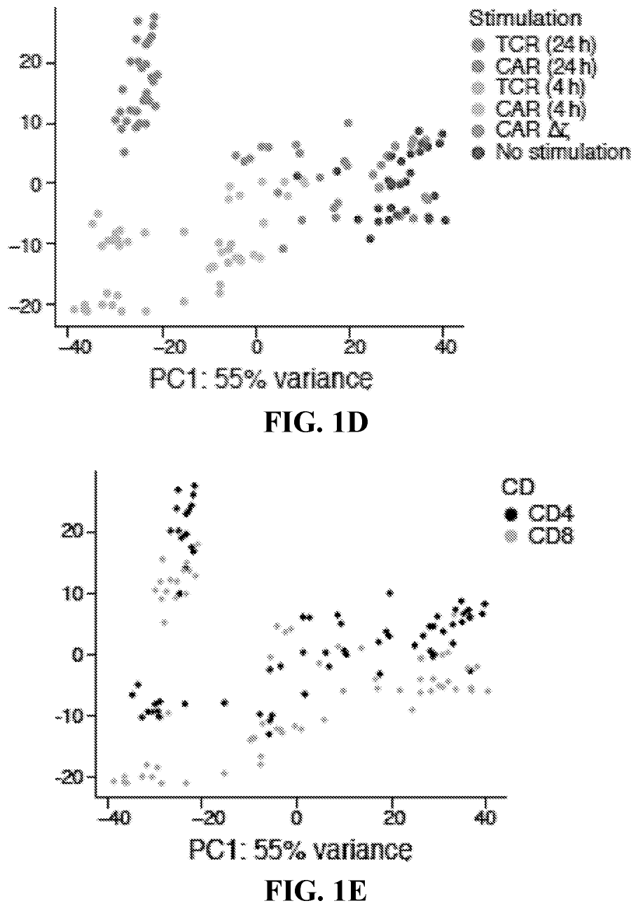

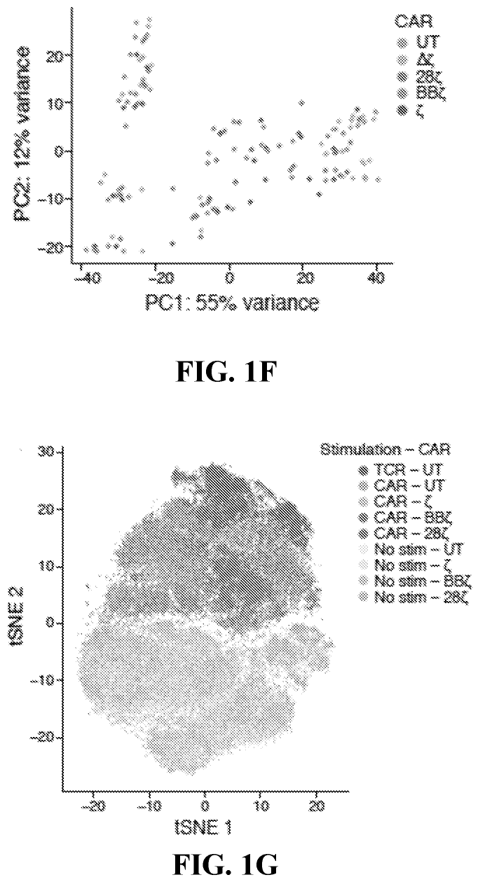

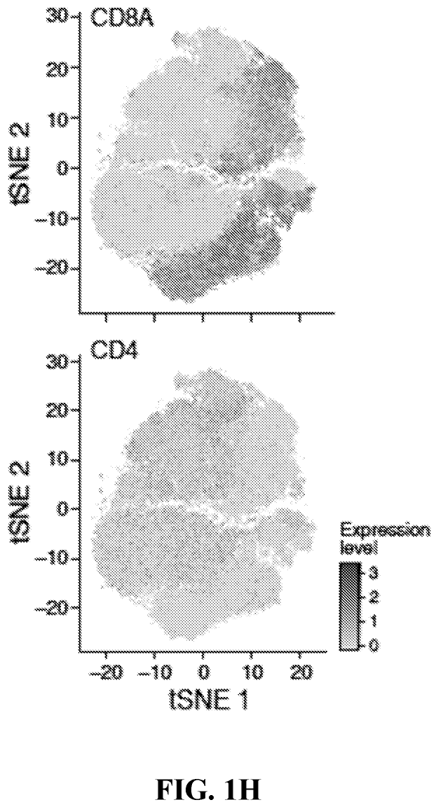

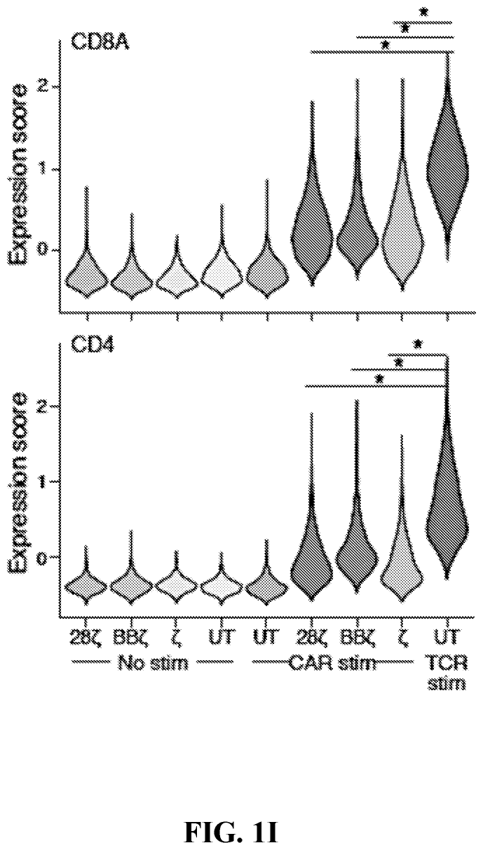

To identify the transcriptional differences downstream of various CARs expressed in T cells, four different CAR constructs were synthesized bearing distinct signaling domains (FIG. 1A), with established CAR constructs that closely resemble those in clinical use, with slight deviations: a first-generation CAR (ζ) that contained only a CD3ζ signaling domain, two second-generation CARs, one with a CD28 (28ζ) (with a different vector and promotor than axicabtagene ciloleucel and a transmembrane domain was derived from CD8 instead of CD28), and the other with a 4-1BB (BBζ) co-stimulatory domain (same as the tisagenlecleucel product), and a control CAR construct that contained a truncated, non-signaling CD3ζ chain (Δζ). All CARs had the same single chain variable fragment (scFv) against CD19 with identical CD8 hinge and transmembrane domains. An mCherry fluorescent reporter was included gene downstream of the CAR construct to facilitate evaluation of CAR transduction. Aside from the co-stimulation domains, all components of these CAR T cells were kept identical so we could interrogate changes in signaling domains. To extend the scope of our findings, we also synthesized CARs with a different antigen-binding domain, using an anti-EGFR scFv based on cetuximab, but keeping all other components the same (FIG. 1A). These EGFR CARs were used to validate our key findings from the CD19 CAR T cells' profiles.

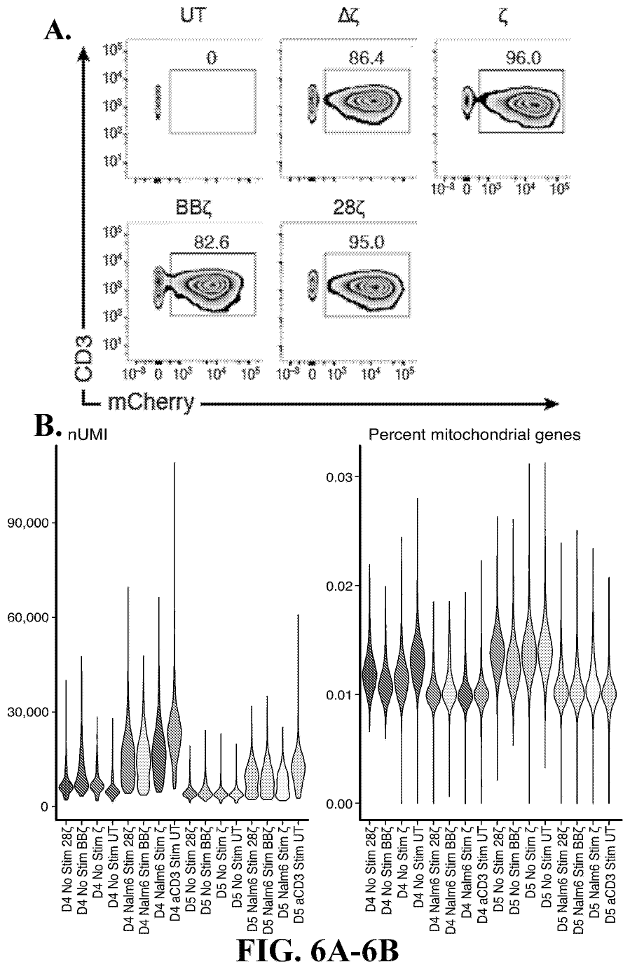

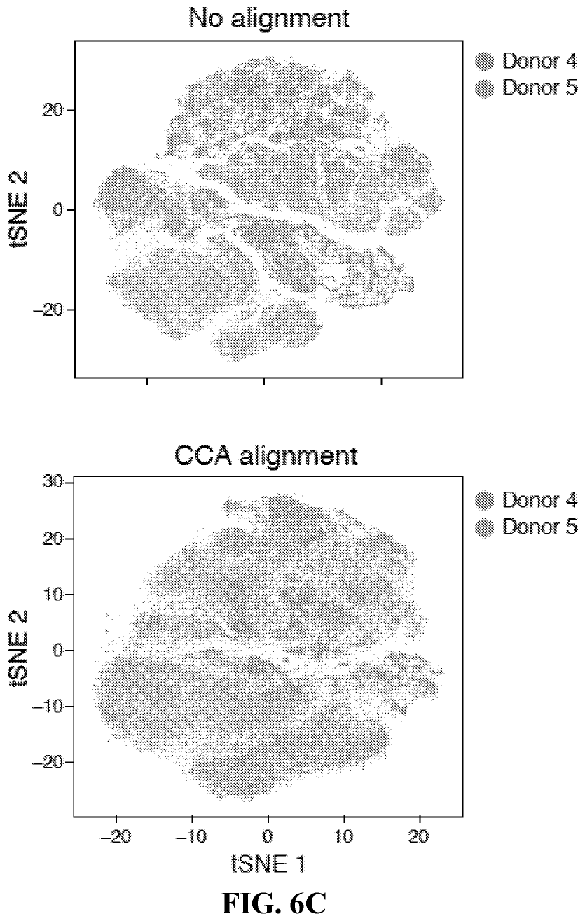

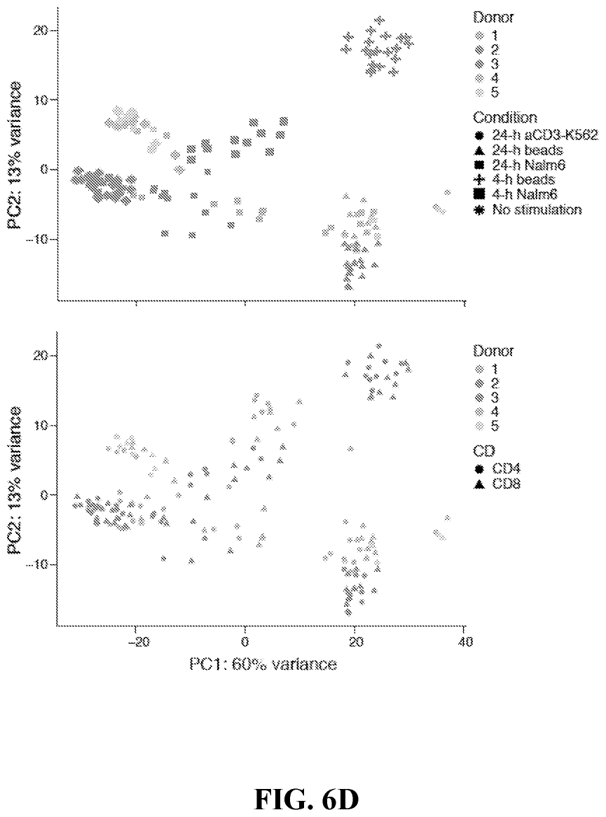

To generate CAR T cells, CD4⁺ and CD8⁺ primary T cells were isolated from the peripheral blood of healthy donors and mixed these two populations in equal proportions prior to activation through the TCR with anti-CD3/anti-CD28 beads (FIG. 1B). The following day, bulk T cells were transduced with lentivirus to express one of the four CD19-specific CAR constructs (ζ, 28ζ, BBζ and Δζ) or left them untransduced (UT). CAR T cells were expanded for one week and rested them for another week. (Cells were not profiled prior to the initial activation step by stimulation with anti-CD3/anti-CD28 beads, but instead used time in culture to rest T cells as they would be at the time of infusion into a patient.) All T cell cultures had comparable transduction and transgene expression levels as determined by the mCherry surrogate marker (FIG. 6A).

We generated a high quality, bulk RNA-Seq atlas of CD19-targeting CAR T cell profiles separately sorted on CAR⁺CD4⁺ and CAR⁺CD8⁺ T cell populations with each construct (FIG. 1B, Source Data, Methods-Example 11), as well as a scRNA-seq atlas of 83,123 profiles of UT T cells and ζ, 28ζ and BBζ CAR⁺ T cells (FIG. 1C, Source Data, Methods-Example 11). For bulk RNA-Seq (FIG. 1B), we stimulated the CAR T cells for either 4 or 24 hours through their CAR using irradiated Nalm6 leukemia cells (which express CD19 endogenously) or through their TCR using anti-CD3/anti-CD28 beads. For scRNA-Seq (FIG. 1C), we profiled CAR T cells at rest and at 24-hours after stimulation and used UT T cells as a no CAR control. As a positive control, we stimulated UT T cells through their TCR with K562 cells expressing a surface scFv against human CD3 (K562-αCD3). We analyzed 83,123 high quality single cell profiles (Methods-Example 11). Quality was consistent across samples from the same donor but differed by donor and stimulation type, as expected (FIG. 6B). We aligned the datasets and corrected for donor-specific batch effects with canonical correlation analysis²¹ (FIG. 6C, Methods-Examples 11).

### Example 2—Antigen Stimulation of CAR T Cells Through their CAR Yields a Weaker but Similar T Cell Activation Signal Compared to Stimulation Via their TCR

To investigate the main drivers of variation across CAR T cell populations, a performed principal component analysis (PCA) of the bulk profiles was performed, combined with linear modeling to account for donor variation. Samples grouped primarily according to the type of antigen stimulation: unstimulated, stimulated through the CAR, or stimulated through the TCR (FIG. 1D), secondarily by CD4⁺ vs. CD8⁺ cell types (FIG. 1E), and not by the CAR construct expressed (FIG. 1F). The only exception was for Δζ CARs stimulated with CD19-expressing targets, which grouped with unstimulated cells, indicating that lentiviral transduction with Δζ CARs indeed has little to no discernible effect on the T cell transcriptome, and are an appropriate negative control for CAR-mediated signaling (FIG. 1F).

In particular, there was a striking difference in the transcriptional response when human CAR T cells were activated through their TCR compared to their CAR (FIG. 1F). The TCR-activated CAR T cells grouped with TCR activated UT cells and further away from the unstimulated cells on PC1 than the CAR-activated CAR T cells. This is consistent with findings from mouse CAR T cells, where stimulation through the CAR vs. TCR induces distinct gene expression signatures²².

To determine whether these distinctions stem from differences in the percentage of cells being stimulated or the degree or kind of stimulation within each individual cell, the scRNA-Seq profiles were turned to. Overall, the bulk differences in T cell stimulation were also reflected at the single cell profiles, in a manner suggesting an overall shift across the entire population, rather than a change in the proportion of responding cells (FIG. 1G). Furthermore, TCR-stimulated UT cells grouped separately from the CAR-stimulated CAR T cells, whereas such a separation was not observed at rest (FIG. 1G). T cells also separated by CD4⁺ and CD8⁺ cell types, again consistent with the bulk profiles (FIG. 1H). Consistently, pseudobulk CD4⁺ and CD8⁺ profile generated by averaging the respective scRNA-Seq data (Methods-Example 11) included with the true bulk RNA-Seq samples in the PCA with correction for donor variation, separated on PC1 by stimulation (FIG. 6D). Thus, TCR stimulation is distinct from CAR stimulation, regardless of the way in which T cells were activated through the TCR (K562-αCD3 or αCD3/αCD28 bead).

Differential expression analysis of the scRNA-Seq between antigen-stimulated CAR T cells, TCR stimulated UT cells, and unstimulated T cells indicated that stimulation of CAR T cells through their CAR yields a weaker but similar T cell activation signal compared to stimulation of untransduced cells through TCR via αCD3. Comparing genes across cells with the three stimuli, TCR stimulated specific genes were known T cell activation genes (e.g., IFNG, IL3, and CCL4; Table 3 herein), those specific to resting CAR and UT T cells were known resting T cell genes (e.g., IL7R), but CAR-stimulated CAR T cell genes were less clearly associated with T cell activation, except for IL2RA. Nevertheless, 75% of the genes upregulated in antigen-activated CAR T cells compared to unstimulated T cells, are among genes induced in TCR stimulated cells (Table 4 herein), suggesting that antigen-CAR activated CAR T cells have a weaker but similar response to TCR stimulated cells. We used these shared genes to define a T cell activation signature (Table 5 herein). αCD3/TCR-stimulated cells expressed the activation signature at an increased level compared to Nalm6/CAR-stimulated cells (P value <10⁻¹⁶, Wilcoxon test), suggesting cell intrinsic differences in activation (FIG. 1I).

TABLE 3
Top 20 genes exclusively defining T cells grouped by
stimulation: anti-CD3 stim vs Nalm6 stim vs No
stim(except UTD CD19 stim) from the scRNA-seq data.

| Top 20 Genes defining populations | Column1 | Column2 |
|---|---|---|
| aCD3 Stim | Nalm6 Stim | No Stim |
| IFNG | IL2RA | CCL5 |
| CCL4 | TUBA1B | CD52 |
| CCL3 | ENO1 | IL7R |
| IL3 | HSPD1 | BTG1 |
| XCL1 | HSP90AA1 | GIMAP7 |
| CSF2 | HSP90AB1 | S100A4 |
| GZMB | BATF3 | FYB |
| FABP5 | NCL | EVL |
| XCL2 | AC133644.2 | SLFN5 |
| LTA | HNRNPAB | KLF2 |
| LAG3 | RANBP1 | RPS27 |
| MIR155HG | TPI1 | MALAT1 |
| PIM3 | NME1 | MYO1F |
| TNFRSF4 | TXN | CD37 |
| IL13 | CALR | LAPTM5 |
| TNFRSF9 | SRM | FAM65B |
| ZBED2 | RAN | ZFP36L2 |
| PGAM1 | CCND2 | RARRES3 |
| EIF5A | HSPE1 | EVI2B |
| IL5 | TNFSF10 | GNLY |

TABLE 4
Top 20 differentially expressed genes defining Nalm6
Stim CAR T cells vs unstimulated T cells (anti-CD3 stim
excluded from analysis) from the single cell RNA-seq profiles.

Nalm6 Stim
IFNG
IL3
CCL4
XCL1
CSF2
XCL2
CCL3
LTA
GZMB
LAG3
TNFRSF9
PIM3
RGCC
NKG7
FABP5
NDFIP1
MIR155HG
SRGN
PSMA2
BCL2L1

TABLE 5
Gene sets used to calculate gene set scores in single cells.

| T cell | | | Effector | |
|---|---|---|---|---|
| Activation | Tonic Down | Tonic Up | T cell | HLA II |
| IFNG | CD248 | ASB2 | GZMA | CIITA |
| CCL4 | FAM13A | BIRC3 | GZMB | CD74 |
| CCL3 | LTB | CCL3 | PRF1 | HLA-DRB1 |
| IL3 | OPN3 | CCL4 | IFNG | HLA-DRB5 |
| XCL1 | SOCS2 | GGT1 | EOMES | HLA-DMB |
| CSF2 | TNFRSF10A | CTLA4 | | HLA-DPB1 |
| GZMB | PLXNA4 | CSF2RB | | HLA-DQA2 |
| FABP5 | HPCAL1 | GZMB | | HLA-DOA |
| XCL2 | | ZP3 | | HLA-DRA |
| LTA | | SDC4 | | |
| LAG3 | | XCL1 | | |
| MIR155HG | | ZBED2 | | |
| TNFRSF4 | | IFNG | | |
| TNFRSF9 | | | | |
| PIM3 | | | | |

### Example 3—a CD3 Tonic Signaling Signature is Present in Resting CAR T Cells

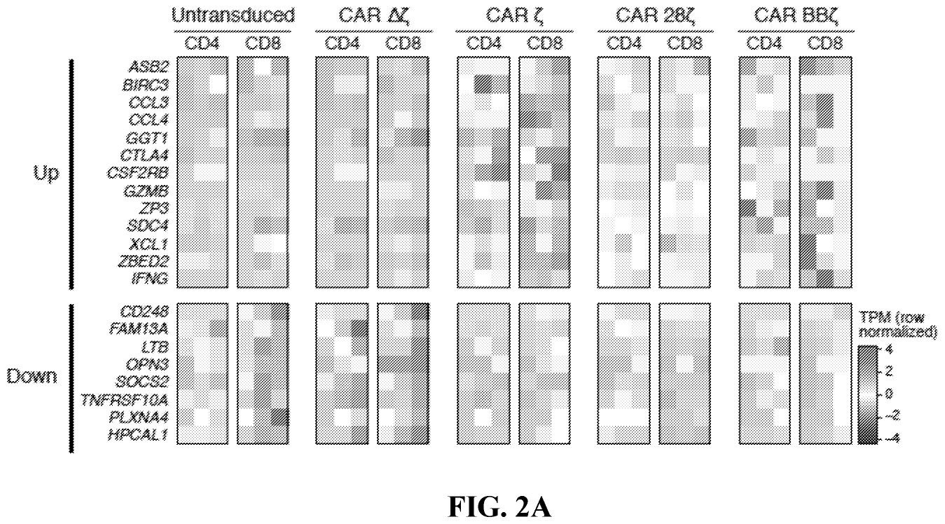

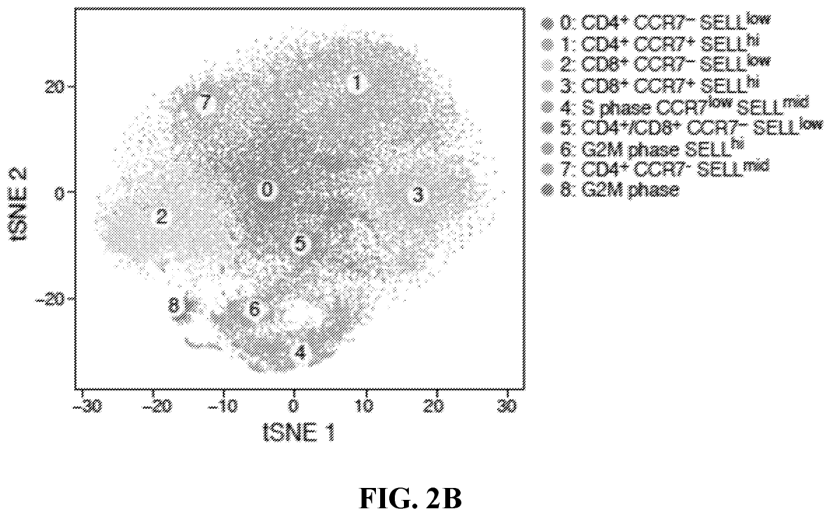

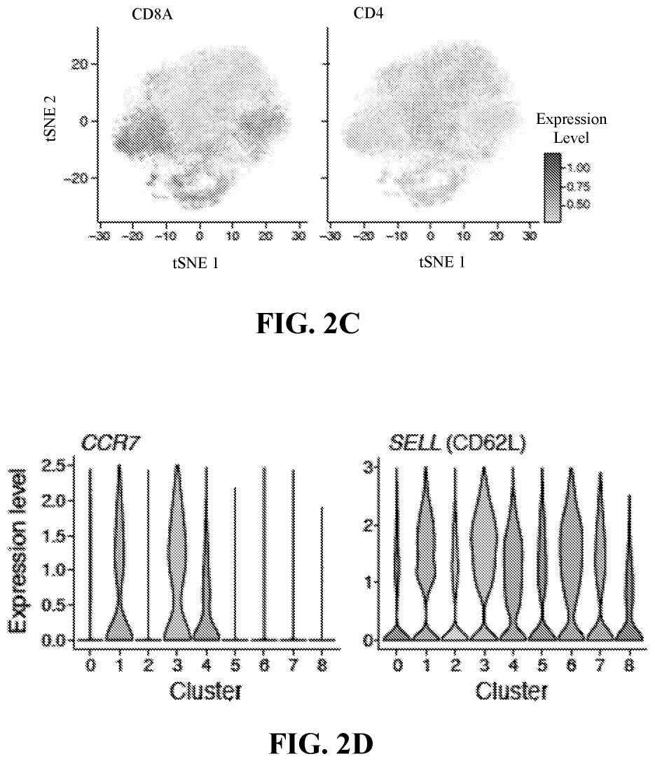

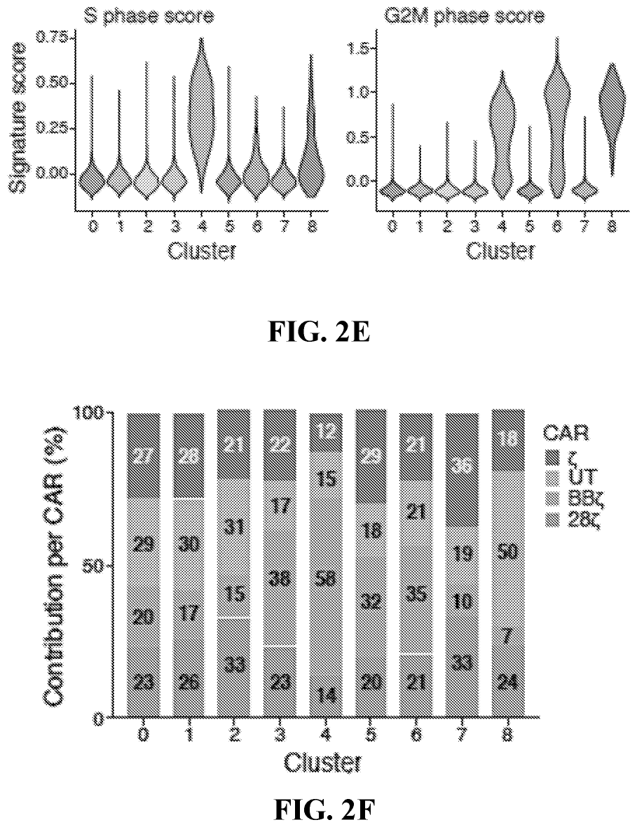

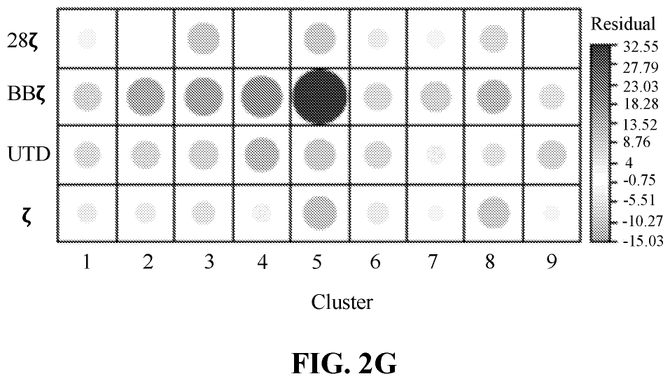

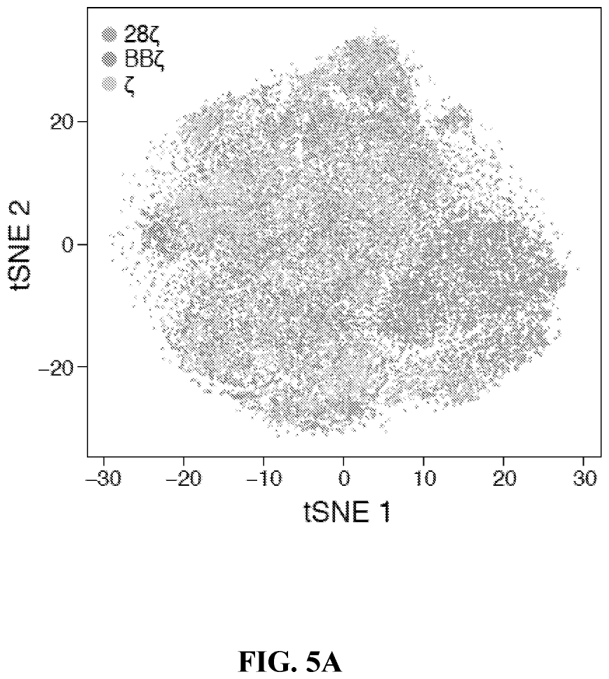

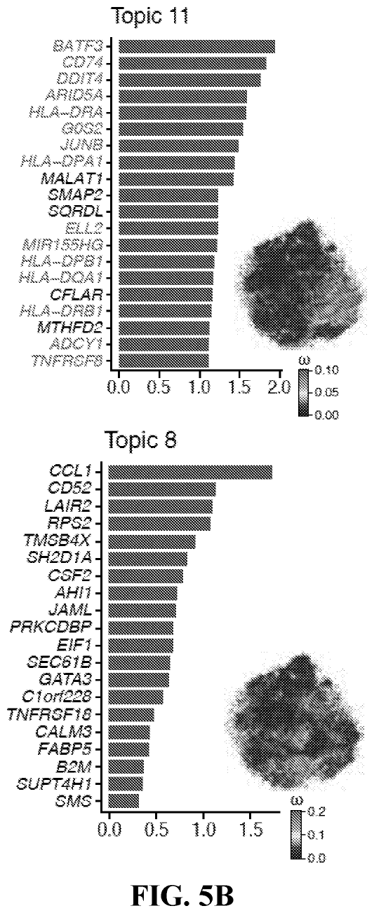

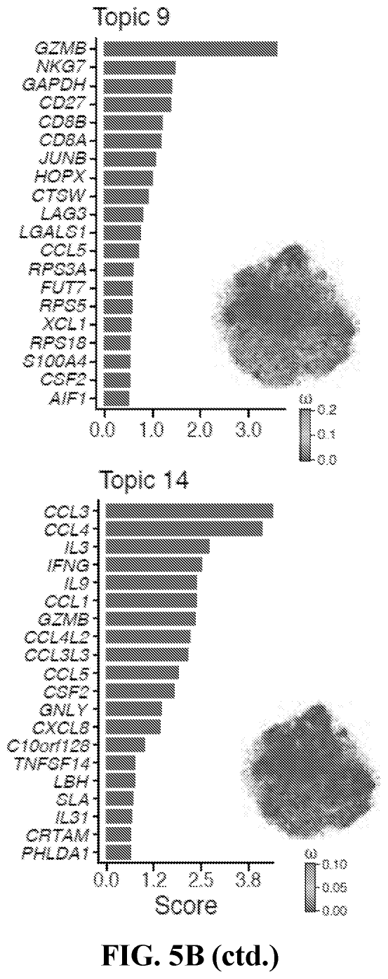

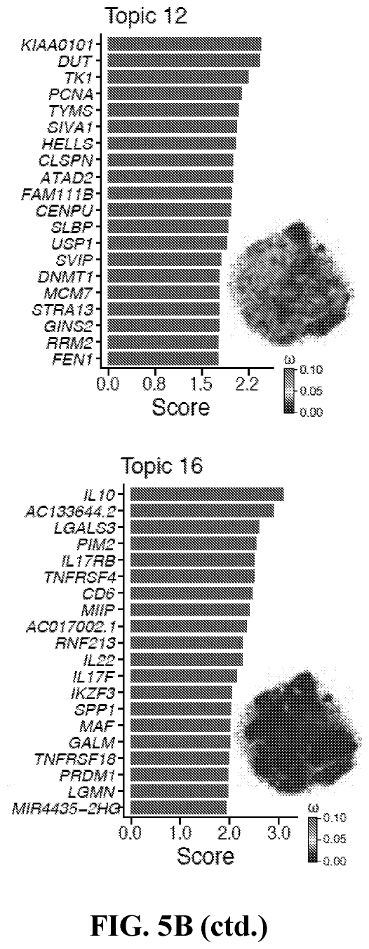

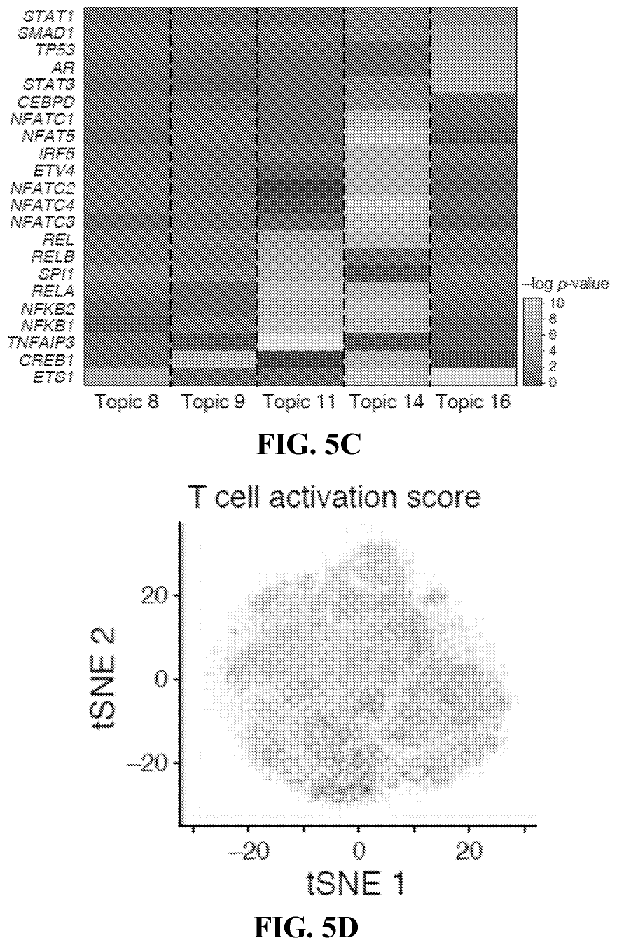

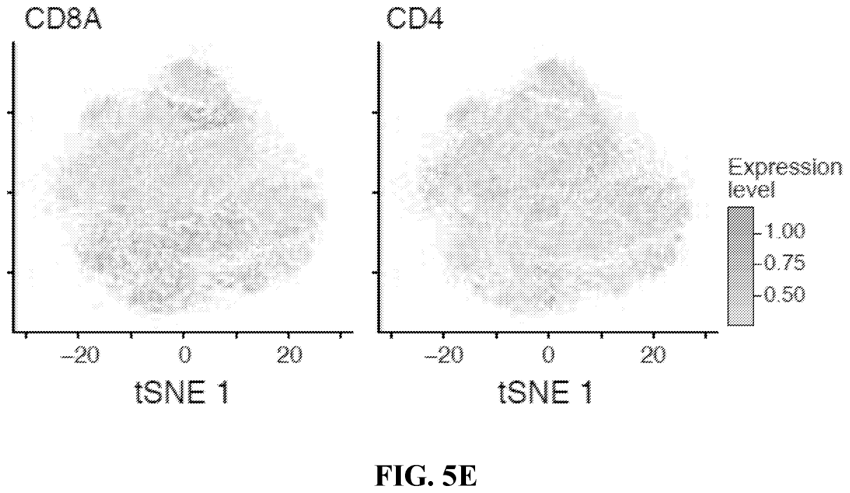

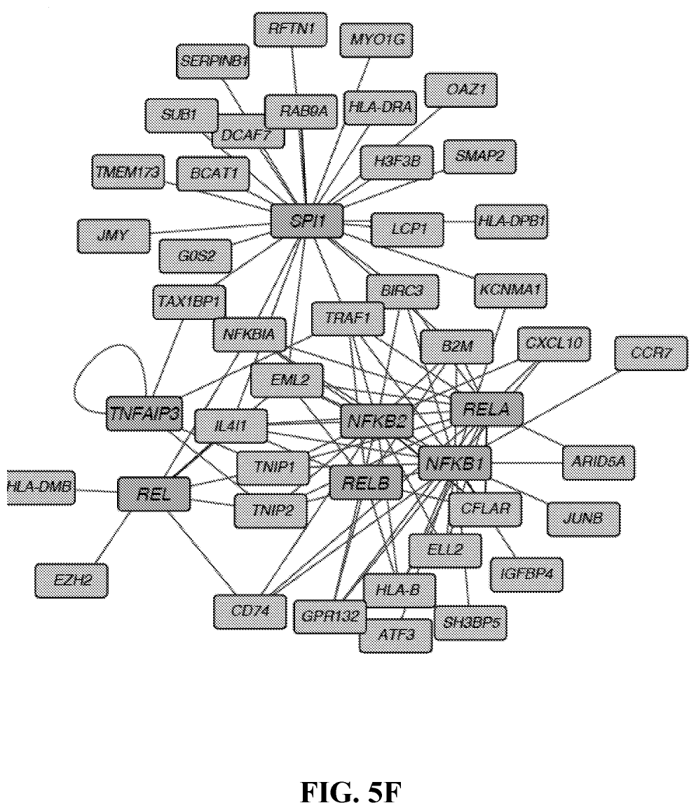

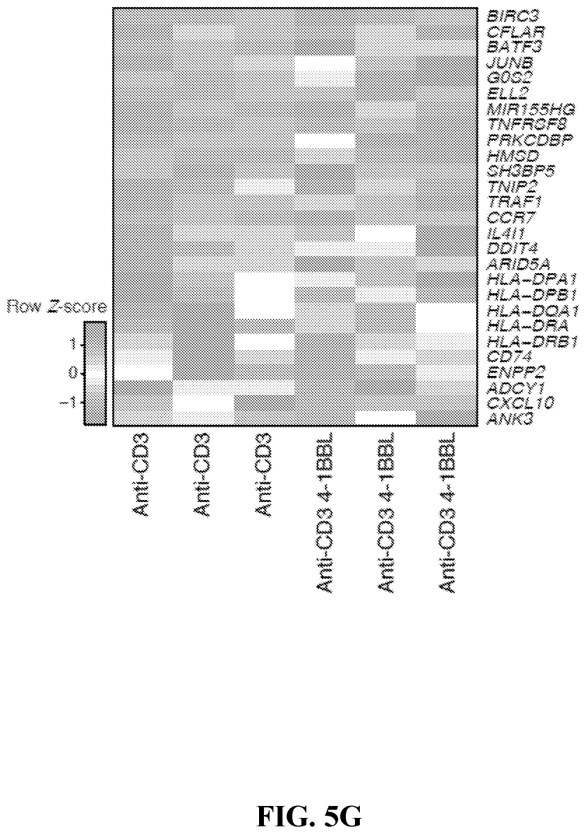

Because CARs in T cells are reported to have a “tonic signaling” effect^{23,24}, the difference among the resting CAR T cells (time 0) were examined next. Based on their bulk profiles, several genes were significantly induced in all functional CARs compared to Δζ (Wald test, Benjami Hochberg FDR <0.05), whereas no genes were significantly differentially expressed between Δζ CAR and UT T cells. Moreover, CARs bearing a CD3ζ chain had a specific, stimulation-independent, transcriptional signature at rest (FIG. 2A) enriched in genes involved in response to cytokine stimulus (FDR=1.51*10⁻⁶; Gene Set Enrichment Analysis (GSEA)), including CCL3 and CCL4, which are involved in monocyte recruitment, and GZMB (encoding granzyme B), a key cytotoxicity gene. This signature was validated in these CARs with αEGFR specificity but the same signaling domains by digital droplet PCR of select up- and downregulated signature genes (FIGS. 5A-5G). Thus, the expression of a CAR in T cells modulates their expression profiles and potentially their phenotype even at rest, prior to CAR-antigen engagement on a tumor cell.

Based on the scRNA-seq profiles, however, the tonic signaling program we identified was present in only a subset of all CAR T cells at rest (FIG. 2C). To characterize this subset, all resting T cell samples were clustered (including UTD) into eight clusters (FIG. 2D), and annotated them by their expression of known surface marker genes (FIG. 2E). Cells with an increased tonic signaling signature were a portion of the CCR7⁻ CD62L⁻ CD8 and CD4 T cells and had increased expression of T cell effector genes (FIG. 2E, Table 5). Thus, even following transduction with a particular CAR construct, there is residual heterogeneity among individual CAR T cells.

### Example 4—Evidence of Tonic Signaling Through the 4-1BB Co-Stimulation Domain

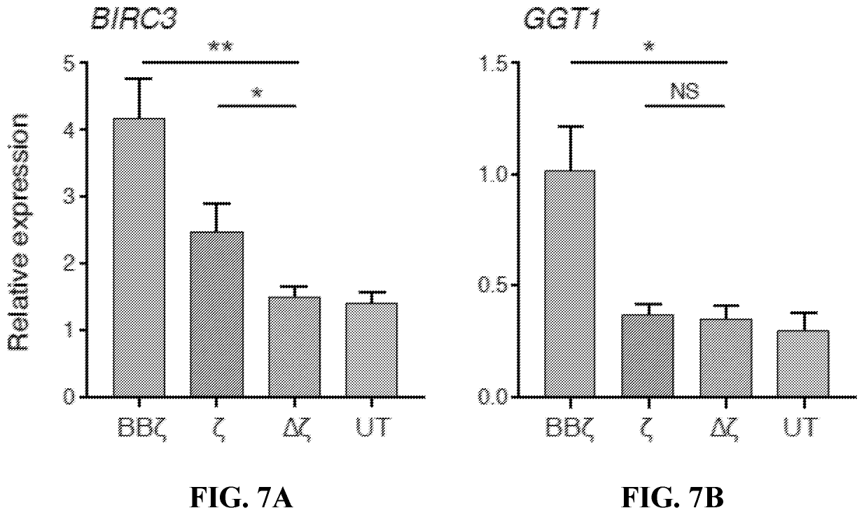

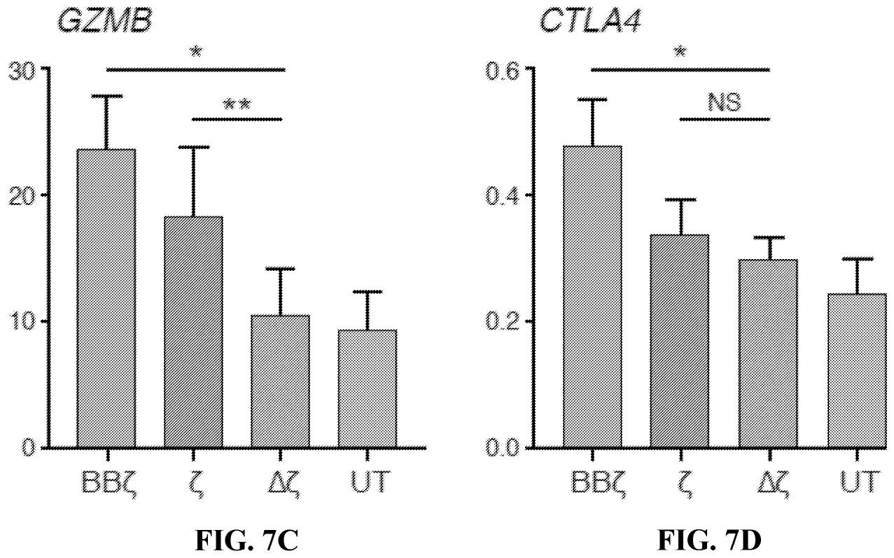

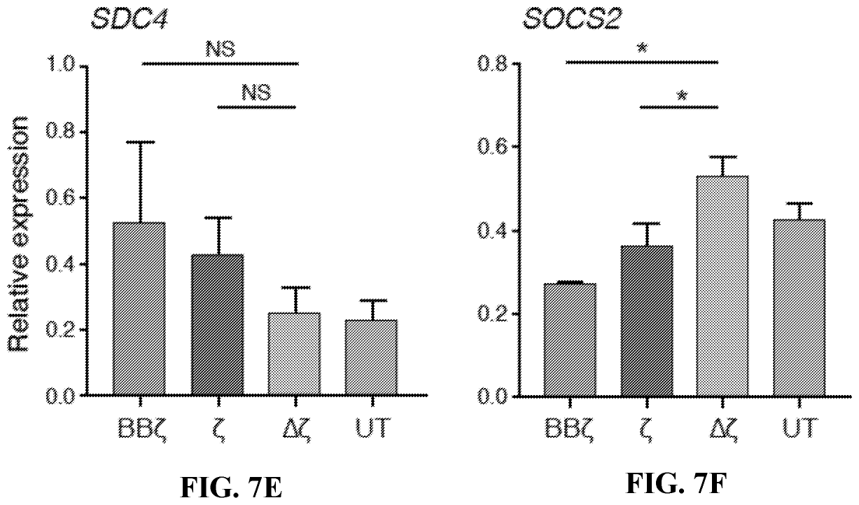

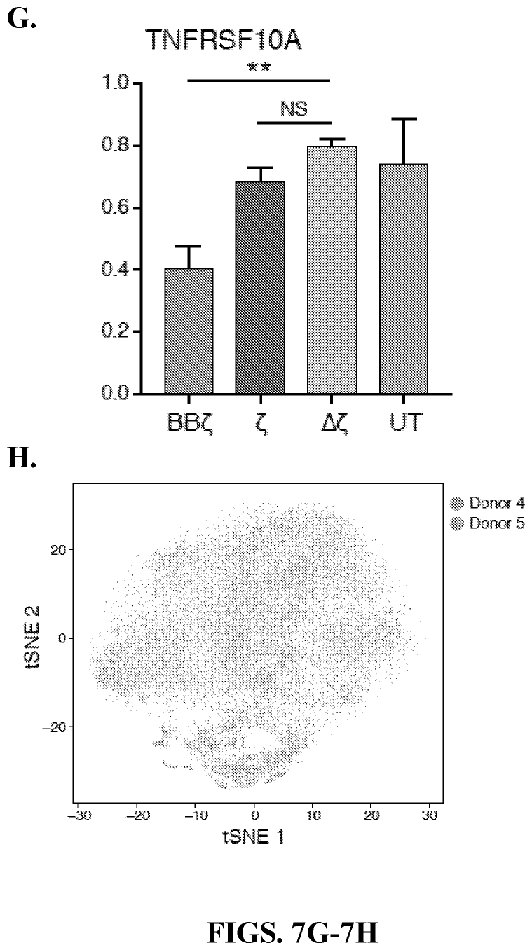

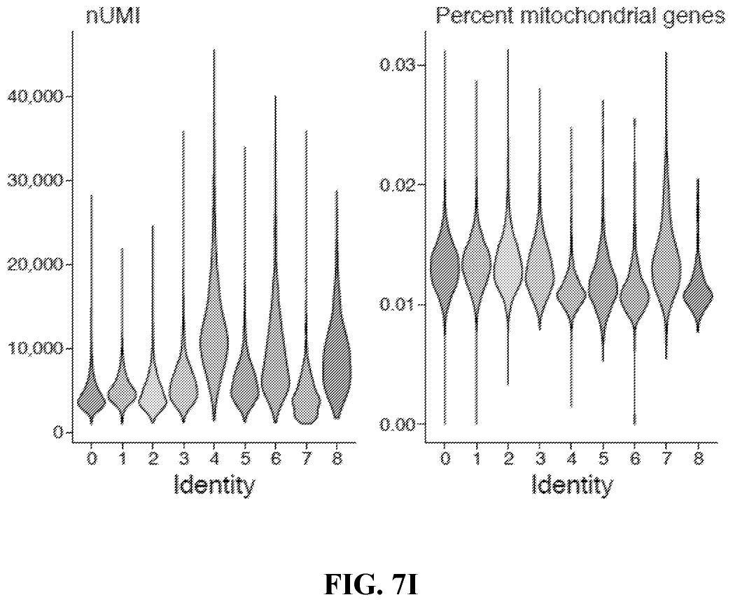

Although tonic signaling of CARs has been reported before, it has mainly been attributed to differences in the binding characteristics of the extracellular portions of the CARs rather than independently through the costimulatory domains^{23,24}. Additional potential contributing factors include the multiple sub populations of T cells present at homeostasis, including CD4⁺ and CD8⁺ T cells, and their respective naïve, effector and different memory subsets^{25,26}. To explore these factors, the single cell profiles were leveraged to estimate the heterogeneity of UT and CAR expressing T cells at rest (prior to any CD19 stimulation through their CAR). The cells from the resting T cell samples (including UT) were clustered into eight clusters (FIG. 2B), annotated them by expression of known marker genes (CD4, CD8A, CCR7 and SELL (CD62L)) (FIG. 2C), and scored them for cell cycle gene signatures (FIG. 2E, FIGS. 7H-7I). Effector memory-like (cluster 2) and central memory-like (cluster 3) were distinguished among CD8 cells and within CD4 cells central memory-like (cluster 1), effector memory-like (cluster 0), and an intermediate population (cluster 7; which expressed CD62L but not CCR7).

### Example 5—Evidence of Tonic Signaling Through the 4-1BB Co-Stimulation Domain

Only 4-1BB containing CAR T cells had a distribution across the clusters that was distinct from all other cells (p-value <2.2*10⁻¹⁶, xx χ² test), and were enriched in CD8 central memory-like (cluster 3) and depleted among CD8 effector memory-like (cluster 2) and CD4 central memory-like (cluster 1) cells (FIGS. 2F-2G and Table 6). Whereas previous studies showed that BBζ CAR T cells are enriched for central memory over effector memory CD8 T cells after CAR stimulation¹⁷, here we find that this enrichment is present prior to stimulation. Thus, the 4-1BB co-stimulation domain may also have tonic signaling effects, distinct from CD3ζ tonic signaling.

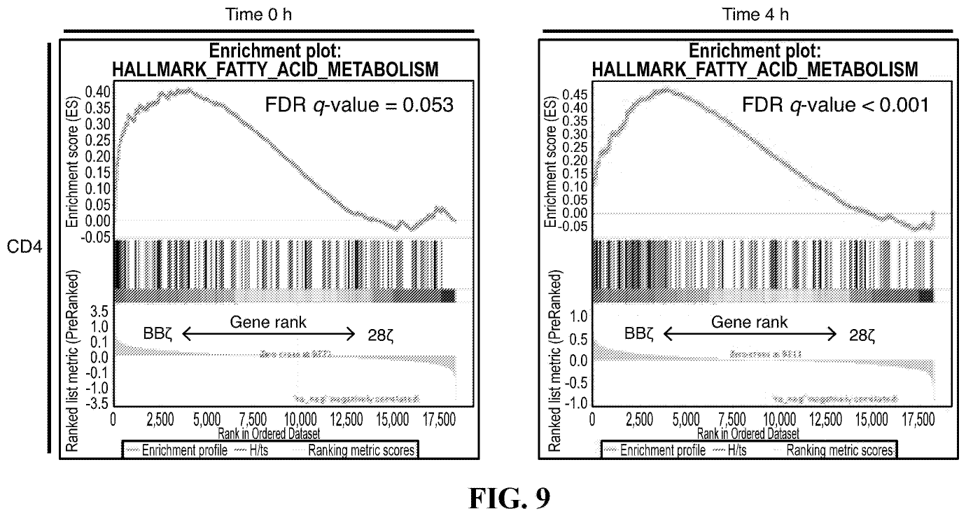

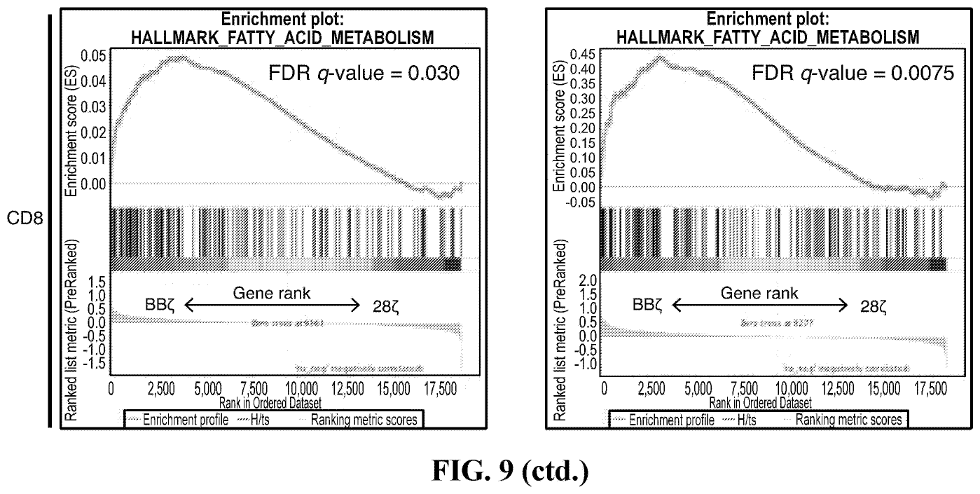

Differentially expressed genes between BBζ and 28ζ bulk RNA-seq profiles of resting CAR T cells (FIG. 8A) further support the hypothesis of tonic signaling from the 4-1BB co-stimulatory domain. Recently, in vitro studies have demonstrated that several days after CAR activation, 28ζ CAR T cells have enhanced glycolytic metabolism, whereas BBζ CAR T cells rely more on fatty acid metabolism¹⁷. Importantly, fatty acid oxidation genes were enriched in BBζ versus 28ζ CAR T cells (FIG. 9) both in CAR T cells at rest and after antigen stimulation. This further illustrates that signaling from the co-stimulatory domain, particularly 4-1BB, can modulate T cell programs even prior to antigen stimulation.

TABLE 6
The contribution of each CAR sample across the clusters
calculated for the single cell data from resting T cell across donor 4
and 5. Cells were clusters by a shared nearest neighbor using the
top 20 PCA dimensions and 0.6 resolution. Underlined text highlights
the clusters enriched for 4-1BB CAR T cells.

| Cluster 0.6res | 28Z | BBZ | UTD | Z |
|---|---|---|---|---|
| 0 | 21.24 | 18.58 | 26.28 | 25.06 |
| 1 | 22.19 | 13.86 | 25.19 | 23.69 |
| 2 | 19.92 | 8.53 | 18.76 | 12.84 |
| 3 | 13.5 | 21.46 | 9.4 | 12.43 |
| 4 | 4.24 | 17.4 | 4.57 | 3.72 |
| 5 | 5.87 | 9.4 | 5.13 | 8.53 |
| 6 | 4.93 | 8.28 | 4.91 | 5.08 |
| 7 | 7.34 | 2.27 | 4.13 | 8.04 |
| 8 | 0.77 | 0.21 | 1.64 | 0.61 |

### Example 6—Antigen Stimulation in BBζ CAR T Cells Results in Persistent Upregulation of an Activation Program Associated MHC Class II Genes, but not PD1

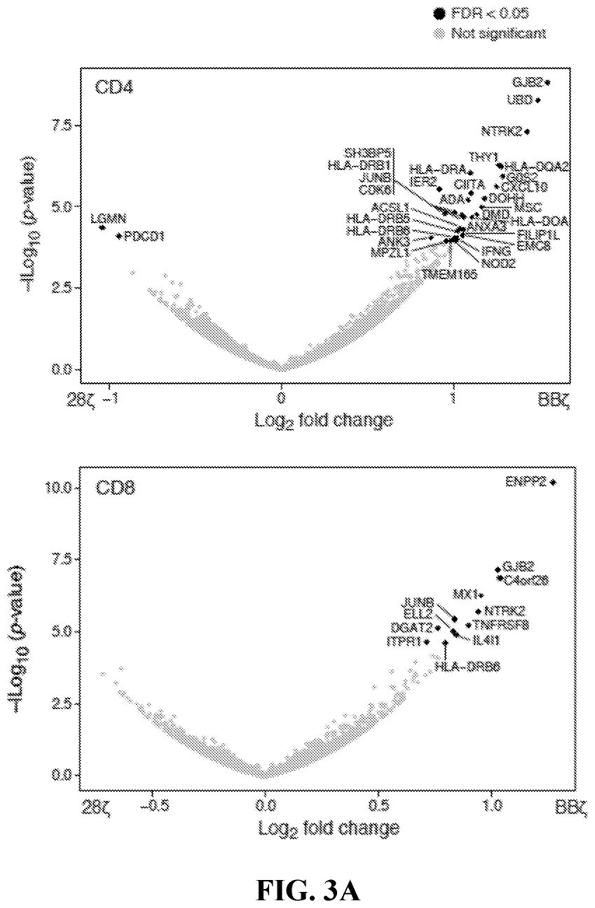

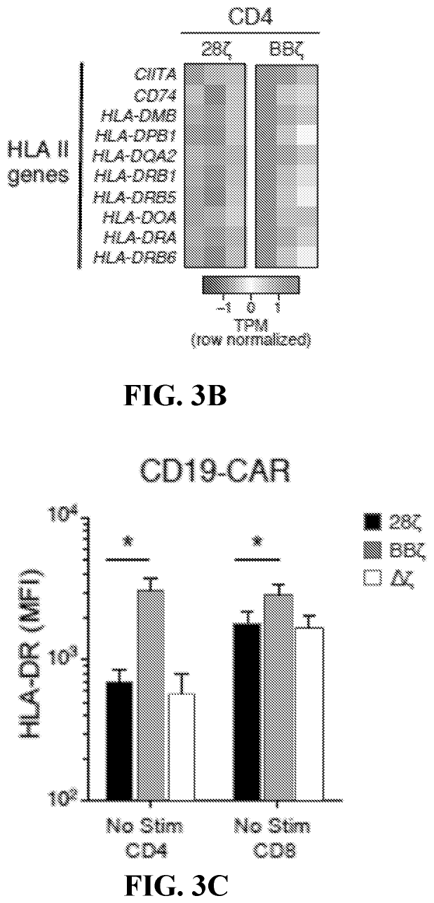

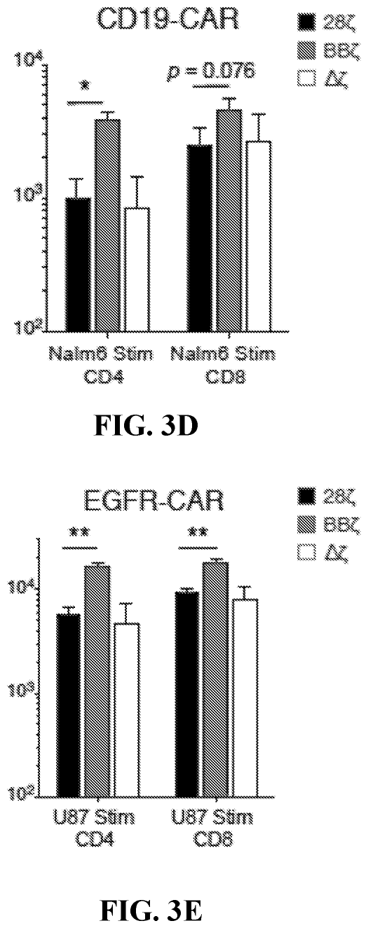

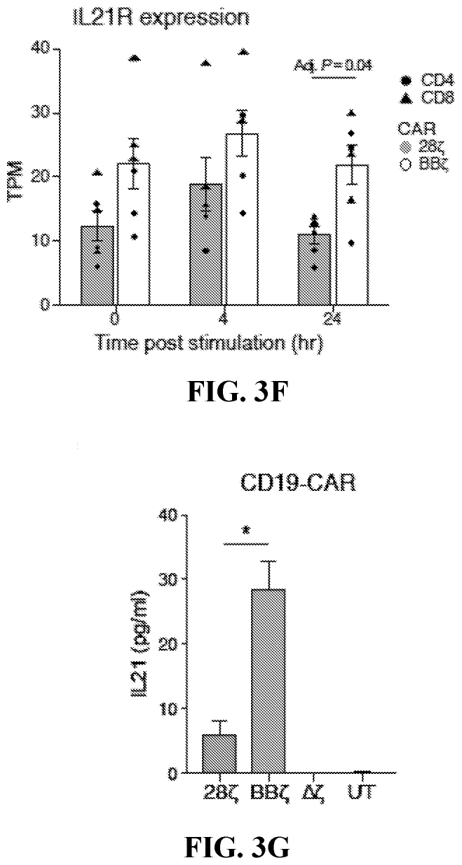

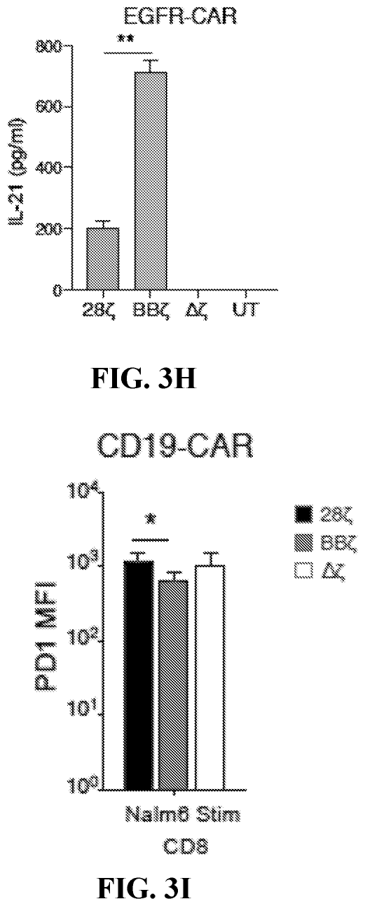

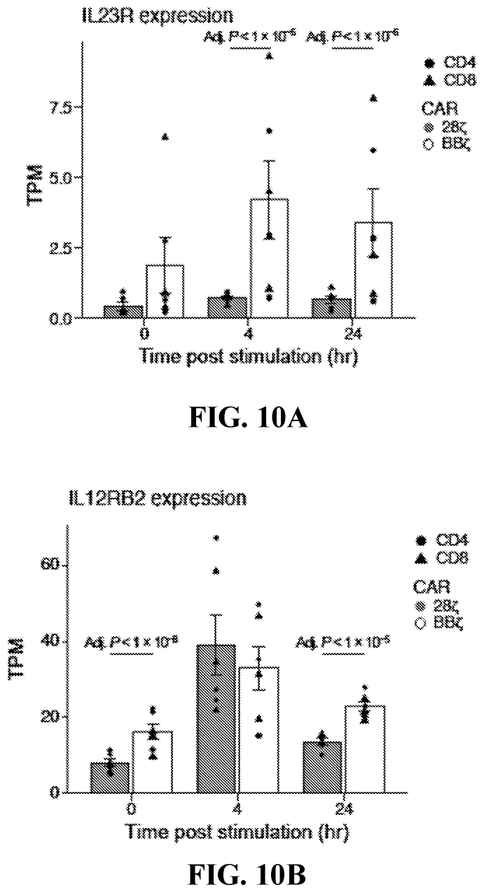

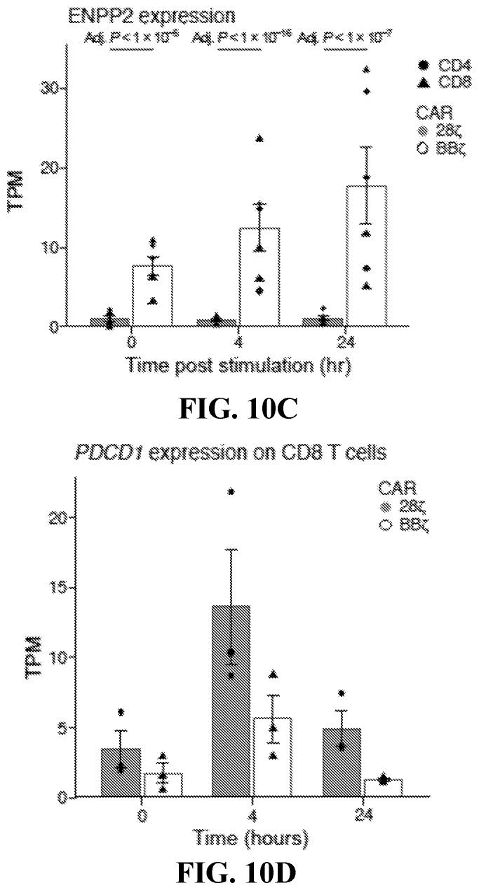

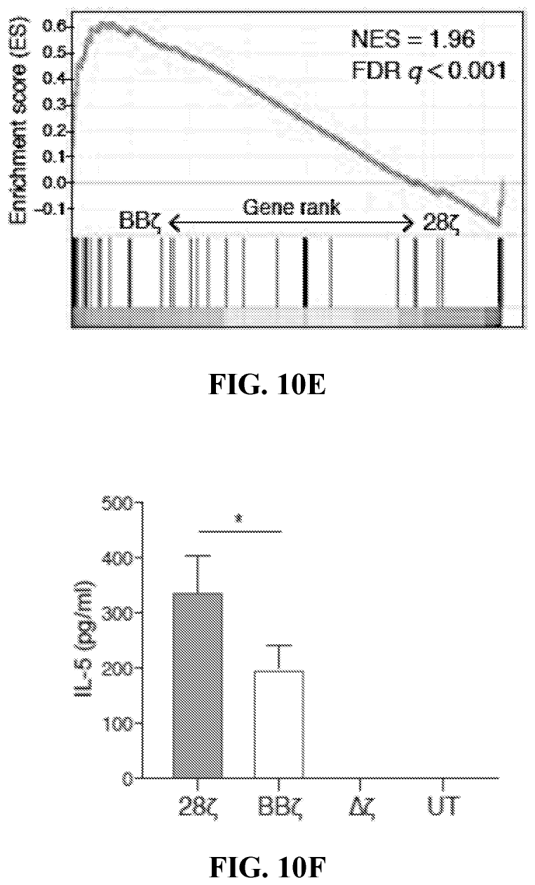

Turning to stimulated CAR T cells, genes up-regulated in BBζ vs. 28ζ CAR T cells based on the bulk profiles (FIG. 3A and FIG. 8A; Table 7), including multiple cytokine and immune signaling pathways (FIG. 8B), especially MHC II genes and their regulators (FIG. 3B). This was validated at the protein level by flow cytometry of HLA-DR expression in anti-CD19 CAR T cells at rest (FIG. 3C) and after activation (FIG. 3D) and in anti-EGFR CAR T cells after activation (FIG. 3E). HLA genes were up regulated in BBζ versus 28ζ CAR T cells at all time points, including at rest, again indicating that ligand-independent signaling of the 4-1BB co-stimulatory domain can modulate gene expression in CAR T cells. TNF and IFNγ signaling genes were also up regulated in BBζ CARs vs. 28ζ at 24 hours post stimulation (Table 8). Furthermore, the cytokines and cytokine receptors IL21, IL21R, IL12RB2, and IL23R were upregulated in BBζ CARs vs. 28ζ CARs, in both CD4⁺ and CD8⁺ T cells (FIGS. 3F and 1-A-10B, by bulk RNA-Seq), and stimulated BBζ CAR T cells produced more soluble IL-21 than 28ζ CARs in both anti-CD19 and anti-EGFR CAR T cells (FIGS. 3G-3H). This axis is particularly important for the formation of long-term memory T cell responses. Finally, ENPP2, encoding autotaxin, was the most significant differentially expressed gene between the two second-generation CAR T cells, induced in all BBζ samples both at rest and following stimulation (FIG. 10C). ENPP2 encodes a phosphodiesterase, which hydrolyzes lysophospholipids to produce lysophosphatidic acid and is involved in chemotaxis and proliferation²⁷.

Conversely, LGMN (encoding asparaginyl endopeptidase) and PDCD1, which encodes the PD1 protein, were the strongest induced genes in stimulated 28ζ vs. BBζ CD4⁺ CARs (FIG. 3A). Though there were no genes in the CD8 group upregulated in 28 ζ CARs that had an adjusted pval<0.1 at 24 hours, PDCD1 had one of the higher gene expression difference between 28ζ vs. BBζ CART cells (FIG. D). PD1 is both an inhibitory receptor and a known marker of T cell activation. This finding was validated using flow cytometry CAR T cells from additional donors and confirmed that surface expression of PD1 was higher in stimulated 28 ζ CARs compared to BBζ CARs (FIG. 3I).

TABLE 7
CAR T Gene Expression Data. Table of select enriched gene sets in MSigDB
with computed overlaps of DE genes upregulated in BBζ compared to
28ζ CARs in CD4+ and CD8+ samples combined. ‘k’ refers to the
number of genes in the intersection of the query set with a set from
MSigDB. ‘K’ refers to the number of genes in the set from MSigDB.
‘FDR q-value’ is false discovery rate analog of hypergeometric
using Benjamini and Hochberg to correct for multiple testing.
CD combined - no stimulation

| Gene | baseMean | log₂FoldChange | p value | p_{adj} |
|---|---|---|---|---|
| IL12RB2 | 277.90 | 0.88 | 2.23E−09 | 2.83E−05 |
| JUN | 71.93 | 1.41 | 4.92E−09 | 3.12E−05 |
| EGR1 | 135.61 | 1.25 | 2.89E−08 | 1.22E−04 |
| CORO7-PAM16 | 16.32 | 1.25 | 2.31E−07 | 7.30E−04 |
| ARID5A | 628.90 | 0.96 | 4.36E−07 | 1.10E−03 |
| WNT5B | 15.10 | 1.11 | 1.67E−06 | 3.52E−03 |
| CDKN1A | 437.24 | 0.81 | 2.24E−06 | 4.05E−03 |
| JAKMIP1 | 472.08 | 0.76 | 2.58E−06 | 4.08E−03 |
| ENPP2 | 71.10 | 1.12 | 3.20E−06 | 4.48E−03 |
| JUNB | 488.86 | 1.00 | 3.54E−06 | 4.48E−03 |
| CHRNA6 | 55.84 | 1.04 | 5.98E−06 | 6.88E−03 |
| C1orf56 | 243.41 | −0.84 | 7.38E−06 | 7.79E−03 |
| FAIM3 | 685.92 | −0.94 | 1.21E−05 | 1.13E−02 |
| FOS | 172.24 | 1.06 | 1.25E−05 | 1.13E−02 |
| MPZL1 | 119.80 | 0.94 | 1.34E−05 | 1.13E−02 |
| VNN2 | 112.96 | −0.97 | 1.80E−05 | 1.42E−02 |
| MPP7 | 47.07 | −0.99 | 1.99E−05 | 1.47E−02 |
| EVI2A | 832.55 | −0.55 | 2.09E−05 | 1.47E−02 |
| DMD | 32.85 | 1.02 | 2.27E−05 | 1.52E−02 |
| CRMP1 | 6.60 | 0.96 | 2.47E−05 | 1.52E−02 |
| IRF8 | 19.52 | 1.01 | 2.87E−05 | 1.77E−02 |
| C4orf26 | 139.77 | 0.88 | 2.94E−05 | 1.77E−02 |
| GCA | 153.30 | 0.77 | 3.34E−05 | 1.92E−02 |
| BATF3 | 87.94 | 1.00 | 3.60E−05 | 1.98E−02 |
| EGR2 | 38.66 | 0.99 | 4.03E−05 | 2.13E−02 |
| EGR3 | 9.42 | 0.95 | 6.07E−05 | 3.07E−02 |
| SH3YL1 | 103.50 | −0.85 | 6.51E−05 | 3.09E−02 |
| GIMAP2 | 848.34 | −0.49 | 6.59E−05 | 3.09E−02 |
| NLN | 65.85 | 0.72 | 6.84E−05 | 3.09E−02 |
| RPS29 | 6613.67 | −0.43 | 8.80E−05 | 3.80E−02 |
| STMN3 | 66.07 | −0.90 | 9.00E−05 | 3.80E−02 |
| LAIR1 | 436.11 | −0.64 | 9.35E−05 | 3.82E−02 |
| ENOX1 | 14.01 | 0.80 | 1.05E−04 | 3.98E−02 |
| ICAM1 | 275.35 | 0.61 | 1.06E−04 | 3.98E−02 |
| ANKRD33B | 43.61 | 0.87 | 1.10E−04 | 3.98E−02 |
| PARP3 | 147.64 | −0.72 | 1.14E−04 | 3.98E−02 |
| ITPRIPL1 | 123.14 | 0.72 | 1.18E−04 | 3.98E−02 |
| ING4 | 479.95 | −0.69 | 1.20E−04 | 3.98E−02 |
| ARHGAP10 | 101.10 | 0.81 | 1.21E−04 | 3.98E−02 |
| ZNF672 | 242.59 | 0.76 | 1.23E−04 | 3.98E−02 |
| PRDM1 | 607.02 | 0.69 | 1.35E−04 | 4.28E−02 |
| RPL39 | 7265.01 | −0.40 | 1.40E−04 | 4.32E−02 |
| GJB2 | 52.49 | 0.92 | 1.44E−04 | 4.33E−02 |
| FILIP1L | 92.12 | 0.92 | 1.57E−04 | 4.53E−02 |
| ATHL1 | 309.06 | −0.79 | 1.60E−04 | 4.53E−02 |
| FOXP1 | 697.19 | −0.73 | 1.61E−04 | 4.53E−02 |
| MAPKAPK5-AS1 | 481.43 | −0.49 | 1.74E−04 | 4.79E−02 |
| BBS2 | 151.93 | −0.75 | 1.79E−04 | 4.81E−02 |
| ALPK2 | 10.74 | 0.75 | 1.84E−04 | 4.84E−02 |
| AMICA1 | 1918.97 | −0.62 | 1.95E−04 | 4.99E−02 |
| CDCP1 | 85.56 | −0.84 | 1.99E−04 | 4.99E−02 |
| HBEGF | 17.84 | 0.90 | 2.01E−04 | 4.99E−02 |
| SULT1B1 | 30.29 | −0.90 | 2.08E−04 | 5.02E−02 |
| LIF | 34.30 | 0.88 | 2.10E−04 | 5.02E−02 |
| CDK6 | 769.45 | 0.72 | 2.14E−04 | 5.03E−02 |
| C16orf54 | 1320.99 | −0.51 | 2.28E−04 | 5.25E−02 |
| EVI2B | 1752.86 | −0.44 | 2.53E−04 | 5.62E−02 |
| MINA | 529.77 | 0.68 | 2.60E−04 | 5.62E−02 |
| SLC16A3 | 201.90 | 0.71 | 2.65E−04 | 5.62E−02 |
| LOC728875 | 80.09 | −0.74 | 2.68E−04 | 5.62E−02 |
| CIITA | 414.44 | 0.84 | 2.73E−04 | 5.62E−02 |
| PIK3IP1 | 1148.82 | −0.68 | 2.79E−04 | 5.62E−02 |
| GNA15 | 322.20 | 0.85 | 2.82E−04 | 5.62E−02 |
| CTTNBP2NL | 29.00 | 0.87 | 2.83E−04 | 5.62E−02 |
| HLA-DQA2 | 611.02 | 0.85 | 2.84E−04 | 5.62E−02 |
| ABLIM1 | 762.65 | −0.70 | 3.07E−04 | 5.91E−02 |
| RRN3P1 | 44.58 | −0.81 | 3.12E−04 | 5.91E−02 |
| LINC00599 | 23.86 | −0.86 | 3.16E−04 | 5.91E−02 |
| IL16 | 2572.00 | −0.47 | 3.17E−04 | 5.91E−02 |
| P2RY14 | 39.72 | 0.87 | 3.28E−04 | 6.01E−02 |
| PRKCQ-AS1 | 318.17 | −0.73 | 3.32E−04 | 6.01E−02 |
| ADCY1 | 50.15 | 0.86 | 3.54E−04 | 6.30E−02 |
| GPA33 | 74.95 | −0.83 | 3.60E−04 | 6.33E−02 |
| TNFSF10 | 1417.90 | 0.66 | 3.74E−04 | 6.48E−02 |
| FAM200B | 115.05 | −0.61 | 4.06E−04 | 6.93E−02 |
| TCEA3 | 21.95 | −0.83 | 4.11E−04 | 6.93E−02 |
| TTC39C | 805.09 | −0.66 | 4.48E−04 | 7.39E−02 |
| TNFRSF8 | 78.55 | 0.85 | 4.49E−04 | 7.39E−02 |
| MEGF6 | 58.84 | −0.80 | 4.57E−04 | 7.42E−02 |
| ANKRD37 | 28.11 | 0.84 | 5.50E−04 | 8.81E−02 |
| NTRK2 | 46.83 | 0.75 | 5.76E−04 | 9.11E−02 |
| RALB | 527.07 | 0.67 | 5.90E−04 | 9.19E−02 |
| SNHG6 | 583.78 | −0.51 | 5.95E−04 | 9.19E−02 |
| ANXA2R | 272.59 | −0.65 | 6.04E−04 | 9.21E−02 |
| PTBP1 | 866.25 | 0.57 | 6.28E−04 | 9.47E−02 |
| MIR155HG | 213.88 | 0.74 | 6.40E−04 | 9.54E−02 |
| SOCS3 | 76.68 | 0.80 | 6.76E−04 | 9.85E−02 |
| ZC4H2 | 155.84 | −0.65 | 6.78E−04 | 9.85E−02 |
| SERINC5 | 1598.98 | −0.64 | 6.95E−04 | 9.85E−02 |
| SLC7A5 | 216.87 | 0.80 | 6.96E−04 | 9.85E−02 |
| FASN | 135.35 | 0.67 | 7.01E−04 | 9.85E−02 |
| CYB5A | 241.31 | 0.64 | 7.12E−04 | 9.87E−02 |
| SDC4 | 208.05 | 0.56 | 7.18E−04 | 9.87E−02 |
| PLAGL2 | 162.77 | 0.56 | 7.29E−04 | 9.92E−02 |
| Gene | baseMean | log2FoldChange | p value | padj |

CD combined −4 hours

| | | | | |
|---|---|---|---|---|
| ENPP2 | 112.75 | 1.66 | 9.27E−16 | 1.36E−11 |
| ENOX1 | 38.64 | 1.51 | 9.76E−14 | 7.14E−10 |
| DDIT4 | 1679.48 | 1.06 | 2.31E−08 | 1.13E−04 |
| JUNB | 1418.50 | 0.78 | 8.58E−08 | 2.51E−04 |
| CIITA | 408.87 | 1.04 | 7.67E−08 | 2.51E−04 |
| DMD | 176.23 | 1.09 | 1.03E−07 | 2.51E−04 |
| GJB2 | 357.82 | 1.06 | 1.63E−07 | 3.41E−04 |
| ARHGAP10 | 110.52 | 1.00 | 3.80E−07 | 6.94E−04 |
| HLA-DQA2 | 590.33 | 1.03 | 4.88E−07 | 7.94E−04 |
| GNA15 | 859.52 | 0.74 | 8.49E−07 | 1.19E−03 |
| EGR1 | 180.54 | 0.92 | 9.38E−07 | 1.19E−03 |
| JUN | 102.01 | 0.93 | 9.78E−07 | 1.19E−03 |
| LOC100129034 | 47.25 | −0.87 | 2.39E−06 | 2.52E−03 |
| POU2F2 | 157.41 | −0.81 | 2.41E−06 | 2.52E−03 |
| VOPP1 | 625.77 | 0.64 | 2.69E−06 | 2.62E−03 |
| TPM4 | 4616.52 | 0.46 | 3.06E−06 | 2.80E−03 |
| E2F1 | 69.21 | 0.76 | 3.79E−06 | 3.26E−03 |
| PLAUR | 77.17 | 0.91 | 4.84E−06 | 3.94E−03 |
| IL23R | 42.63 | 0.93 | 5.99E−06 | 4.62E−03 |
| CA2 | 24.93 | 0.91 | 7.21E−06 | 5.27E−03 |
| BCL2A1 | 523.29 | −0.84 | 9.50E−06 | 6.62E−03 |
| HLA-DPB1 | 2124.15 | 0.84 | 1.22E−05 | 7.57E−03 |
| HLA-DRB5 | 1112.66 | 0.88 | 1.24E−05 | 7.57E−03 |
| FILIP1L | 154.94 | 0.89 | 1.18E−05 | 7.57E−03 |
| DNAJC6 | 32.64 | 0.88 | 1.31E−05 | 7.69E−03 |
| ATHL1 | 480.50 | −0.74 | 1.41E−05 | 7.95E−03 |
| UBAC1 | 143.89 | 0.64 | 1.82E−05 | 9.50E−03 |
| NR5A2 | 26.90 | 0.87 | 1.79E−05 | 9.50E−03 |
| NTRK2 | 323.52 | 0.88 | 1.95E−05 | 9.84E−03 |
| HLA-DRB6 | 383.18 | 0.86 | 2.20E−05 | 1.07E−02 |
| LZTFL1 | 26.59 | −0.87 | 2.40E−05 | 1.13E−02 |
| BTN2A2 | 160.92 | 0.73 | 2.48E−05 | 1.14E−02 |
| UBE2F | 1143.94 | −0.50 | 2.74E−05 | 1.21E−02 |
| ENPP1 | 57.66 | −0.65 | 2.83E−05 | 1.22E−02 |
| ANKRD33B | 91.63 | 0.81 | 3.08E−05 | 1.29E−02 |
| LRRC32 | 48.70 | −0.85 | 3.42E−05 | 1.32E−02 |
| HLA-DRA | 7237.39 | 0.83 | 3.43E−05 | 1.32E−02 |
| LHFP | 45.16 | 0.86 | 3.43E−05 | 1.32E−02 |
| HLA-DRB1 | 3891.74 | 0.81 | 4.16E−05 | 1.56E−02 |
| ZNF704 | 25.26 | −0.84 | 4.70E−05 | 1.63E−02 |
| TXLNG | 306.77 | −0.48 | 4.80E−05 | 1.63E−02 |
| ADA | 518.05 | 0.82 | 4.69E−05 | 1.63E−02 |
| GCSAM | 67.90 | 0.84 | 4.61E−05 | 1.63E−02 |
| C4orf26 | 149.58 | 0.77 | 4.91E−05 | 1.63E−02 |
| CTH | 32.00 | 0.83 | 5.16E−05 | 1.68E−02 |
| ADRBK1 | 355.88 | 0.51 | 6.60E−05 | 2.02E−02 |
| G0S2 | 83.09 | 0.80 | 6.61E−05 | 2.02E−02 |
| HLA-DPA1 | 5084.66 | 0.81 | 6.61E−05 | 2.02E−02 |
| CD74 | 26227.53 | 0.69 | 6.87E−05 | 2.05E−02 |
| IL18RAP | 81.80 | −0.82 | 7.16E−05 | 2.09E−02 |
| ULBP2 | 14.34 | 0.75 | 7.87E−05 | 2.21E−02 |
| F8 | 54.32 | 0.75 | 7.85E−05 | 2.21E−02 |
| HLA-DOA | 266.79 | 0.81 | 8.15E−05 | 2.25E−02 |
| ARNTL2 | 170.24 | 0.64 | 8.73E−05 | 2.30E−02 |
| RNF19B | 63.25 | 0.69 | 8.61E−05 | 2.30E−02 |
| IL4I1 | 218.73 | 0.76 | 8.79E−05 | 2.30E−02 |
| TMEM178B | 38.27 | 0.74 | 9.02E−05 | 2.32E−02 |
| ODC1 | 476.86 | −0.66 | 1.15E−04 | 2.91E−02 |
| NEK6 | 93.44 | −0.76 | 1.27E−04 | 3.13E−02 |
| TBL1X | 180.42 | −0.72 | 1.28E−04 | 3.13E−02 |
| LINC00176 | 22.63 | 0.77 | 1.48E−04 | 3.55E−02 |
| MED12L | 44.55 | 0.78 | 1.51E−04 | 3.55E−02 |
| DBNDD2 | 33.37 | 0.78 | 1.56E−04 | 3.62E−02 |
| HBEGF | 53.04 | 0.78 | 1.62E−04 | 3.70E−02 |
| HLA-DQB2 | 79.07 | 0.78 | 1.66E−04 | 3.74E−02 |
| TSHR | 16.64 | 0.75 | 1.70E−04 | 3.77E−02 |
| FSCN1 | 29.41 | 0.70 | 1.74E−04 | 3.80E−02 |
| BACH2 | 229.50 | −0.75 | 1.95E−04 | 4.11E−02 |
| MMD | 150.55 | 0.72 | 1.93E−04 | 4.11E−02 |
| CTTNBP2NL | 40.69 | 0.77 | 1.97E−04 | 4.11E−02 |
| RNF167 | 436.03 | 0.44 | 2.23E−04 | 4.60E−02 |
| GPR132 | 292.35 | 0.69 | 2.38E−04 | 4.84E−02 |
| AMICA1 | 1481.74 | −0.40 | 2.42E−04 | 4.84E−02 |
| ADAT2 | 165.94 | −0.59 | 2.48E−04 | 4.91E−02 |
| GNPDA1 | 761.07 | −0.70 | 2.61E−04 | 5.09E−02 |
| ZNF502 | 11.75 | −0.75 | 2.68E−04 | 5.10E−02 |
| CXCR6 | 905.34 | −0.64 | 2.66E−04 | 5.10E−02 |
| BCL2L11 | 491.95 | −0.74 | 2.87E−04 | 5.39E−02 |
| PP7080 | 156.01 | −0.53 | 3.12E−04 | 5.78E−02 |
| C10orf54 | 980.19 | −0.64 | 3.25E−04 | 5.94E−02 |
| OSM | 1149.27 | 0.70 | 3.45E−04 | 6.23E−02 |
| ANK3 | 328.00 | 0.68 | 3.60E−04 | 6.42E−02 |
| EPDR1 | 16.65 | 0.69 | 3.65E−04 | 6.44E−02 |
| MINA | 821.25 | 0.44 | 3.83E−04 | 6.60E−02 |
| PON2 | 98.42 | 0.70 | 3.83E−04 | 6.60E−02 |
| FOXP1 | 905.57 | −0.68 | 3.92E−04 | 6.65E−02 |
| ELL2 | 440.84 | 0.63 | 4.00E−04 | 6.65E−02 |
| P2RY14 | 60.83 | 0.73 | 3.99E−04 | 6.65E−02 |
| WWTR1 | 25.58 | 0.73 | 4.15E−04 | 6.82E−02 |
| ANXA3 | 31.11 | 0.60 | 4.25E−04 | 6.90E−02 |
| ENPP3 | 6.23 | −0.58 | 4.40E−04 | 7.01E−02 |
| DDX4 | 7.56 | 0.60 | 4.38E−04 | 7.01E−02 |
| USP18 | 108.69 | 0.67 | 4.76E−04 | 7.50E−02 |
| ZDHHC9 | 129.72 | −0.63 | 4.95E−04 | 7.71E−02 |
| BAG1 | 122.34 | −0.45 | 5.02E−04 | 7.74E−02 |
| KIF1A | 8.18 | 0.58 | 5.23E−04 | 7.97E−02 |
| TBKBP1 | 17.34 | −0.71 | 5.46E−04 | 8.09E−02 |
| KIAA1671 | 93.00 | 0.65 | 5.40E−04 | 8.09E−02 |
| ADCY1 | 48.55 | 0.71 | 5.47E−04 | 8.09E−02 |
| TMEM189 | 328.90 | 0.38 | 5.81E−04 | 8.51E−02 |
| BAD | 195.51 | 0.50 | 6.76E−04 | 9.80E−02 |
| MTSS1 | 160.55 | 0.70 | 6.94E−04 | 9.96E−02 |

CD combined −24 hours

| | | | | |
|---|---|---|---|---|
| GJB2 | 276.83 | 1.40 | 1.74E−12 | 2.21E−08 |
| NTRK2 | 90.55 | 1.37 | 7.89E−11 | 5.02E−07 |
| JUNB | 1048.37 | 1.03 | 3.34E−10 | 1.42E−06 |
| DGAT2 | 28.53 | 1.26 | 9.61E−10 | 3.05E−06 |
| AMICA1 | 1487.21 | −0.75 | 6.68E−09 | 1.44E−05 |
| MSC | 279.07 | 1.23 | 6.80E−09 | 1.44E−05 |
| SH3BP5 | 220.86 | 1.06 | 9.49E−09 | 1.72E−05 |
| ELL2 | 334.11 | 1.02 | 1.10E−08 | 1.75E−05 |
| DNAJC6 | 34.01 | 1.18 | 2.32E−08 | 3.28E−05 |
| IL12RB2 | 417.28 | 0.60 | 7.84E−08 | 9.97E−05 |
| OAS3 | 789.59 | 0.59 | 8.63E−08 | 9.98E−05 |
| G0S2 | 64.03 | 1.12 | 1.51E−07 | 1.60E−04 |
| HLA-DQA2 | 529.13 | 1.05 | 1.95E−07 | 1.91E−04 |
| DMD | 132.17 | 1.09 | 2.15E−07 | 1.95E−04 |
| HLA-DRB6 | 356.69 | 0.98 | 3.47E−07 | 2.94E−04 |
| FUOM | 112.00 | −0.70 | 3.97E−07 | 3.16E−04 |
| HLA-DRA | 6969.62 | 0.88 | 4.52E−07 | 3.19E−04 |
| IL4I1 | 105.56 | 1.07 | 4.42E−07 | 3.19E−04 |
| ENPP2 | 168.50 | 0.98 | 7.84E−07 | 5.24E−04 |
| P2RY14 | 47.06 | 0.95 | 1.05E−06 | 6.69E−04 |
| C4orf26 | 148.48 | 0.94 | 1.74E−06 | 1.04E−03 |
| ADCY1 | 88.52 | 1.02 | 1.80E−06 | 1.04E−03 |
| MPZL1 | 210.77 | 0.87 | 2.06E−06 | 1.14E−03 |
| PDE4DIP | 808.99 | 0.64 | 2.52E−06 | 1.34E−03 |
| LAIR1 | 305.55 | −0.64 | 2.86E−06 | 1.45E−03 |
| IL23R | 36.02 | 0.99 | 3.31E−06 | 1.62E−03 |
| NFE2L3 | 95.63 | 0.76 | 4.37E−06 | 2.06E−03 |
| ADA | 481.83 | 0.87 | 5.05E−06 | 2.29E−03 |
| ITPR1 | 721.74 | 0.78 | 5.25E−06 | 2.30E−03 |
| HLA-DRB5 | 1052.81 | 0.88 | 6.56E−06 | 2.76E−03 |
| TMEM165 | 258.26 | 0.90 | 6.79E−06 | 2.76E−03 |
| HLA-DPA1 | 5123.01 | 0.80 | 6.95E−06 | 2.76E−03 |
| PDE4A | 94.22 | −0.79 | 7.50E−06 | 2.89E−03 |
| HLA-DPB1 | 2070.98 | 0.80 | 9.49E−06 | 3.55E−03 |
| HLA-DRB1 | 3596.31 | 0.82 | 1.40E−05 | 4.93E−03 |
| ZFAND5 | 577.02 | 0.85 | 1.39E−05 | 4.93E−03 |
| MINA | 699.67 | 0.69 | 1.46E−05 | 5.01E−03 |
| RALB | 847.42 | 0.59 | 1.51E−05 | 5.06E−03 |
| PRKCDBP | 158.64 | 0.86 | 1.72E−05 | 5.61E−03 |
| TMEM178B | 48.02 | 0.91 | 1.90E−05 | 6.04E−03 |
| DGCR6L | 550.79 | −0.50 | 1.99E−05 | 6.08E−03 |
| ARHGEF10 | 26.81 | 0.91 | 2.01E−05 | 6.08E−03 |
| ANK3 | 227.89 | 0.84 | 2.31E−05 | 6.30E−03 |
| TNFRSF8 | 332.10 | 0.87 | 2.36E−05 | 6.30E−03 |
| EHD4 | 306.14 | 0.68 | 2.24E−05 | 6.30E−03 |
| ARID5A | 1030.46 | 0.78 | 2.30E−05 | 6.30E−03 |
| IL21 | 9.62 | 0.78 | 2.36E−05 | 6.30E−03 |
| SPECC1 | 70.92 | 0.71 | 2.38E−05 | 6.30E−03 |
| CIITA | 530.74 | 0.81 | 2.58E−05 | 6.56E−03 |
| CTTNBP2NL | 49.54 | 0.90 | 2.55E−05 | 6.56E−03 |
| GCSAM | 83.51 | 0.89 | 2.94E−05 | 7.33E−03 |
| SH2D1A | 1698.72 | −0.52 | 3.59E−05 | 8.79E−03 |
| JUN | 167.92 | 0.73 | 4.21E−05 | 1.01E−02 |
| BIRC3 | 2844.67 | 0.61 | 4.53E−05 | 1.07E−02 |
| EMC8 | 165.14 | 0.86 | 4.71E−05 | 1.09E−02 |
| ARHGAP10 | 124.94 | 0.82 | 6.51E−05 | 1.48E−02 |
| C15orf48 | 42.86 | 0.83 | 6.72E−05 | 1.50E−02 |
| FBXO4 | 179.45 | −0.56 | 7.41E−05 | 1.62E−02 |
| KLHDC2 | 337.67 | 0.66 | 8.26E−05 | 1.78E−02 |
| HAGHL | 23.92 | −0.81 | 8.53E−05 | 1.81E−02 |
| UPP1 | 342.62 | −0.68 | 9.27E−05 | 1.93E−02 |
| RNF19B | 51.76 | 0.80 | 9.69E−05 | 1.96E−02 |
| RNASE6 | 39.67 | 0.83 | 9.61E−05 | 1.96E−02 |
| TNIP2 | 209.83 | 0.73 | 1.09E−04 | 2.16E−02 |
| BIK | 15.66 | −0.82 | 1.13E−04 | 2.22E−02 |
| SCML4 | 93.00 | −0.70 | 1.20E−04 | 2.31E−02 |
| USP48 | 370.64 | 0.57 | 1.34E−04 | 2.54E−02 |
| P2RY11 | 22.58 | −0.81 | 1.42E−04 | 2.65E−02 |
| MATN4 | 10.13 | 0.67 | 1.51E−04 | 2.75E−02 |
| NCALD | 356.73 | 0.58 | 1.51E−04 | 2.75E−02 |
| NFKBIE | 63.75 | 0.66 | 1.55E−04 | 2.77E−02 |
| CCDC88A | 61.23 | 0.65 | 1.72E−04 | 3.04E−02 |
| LOC100132891 | 38.70 | 0.77 | 1.75E−04 | 3.06E−02 |
| LHFP | 27.32 | 0.80 | 1.84E−04 | 3.16E−02 |
| MINOS1 | 779.99 | −0.40 | 1.96E−04 | 3.28E−02 |
| COL6A5 | 11.28 | 0.66 | 1.93E−04 | 3.28E−02 |
| HLA-DQB2 | 73.00 | 0.78 | 1.98E−04 | 3.28E−02 |
| KCNA3 | 320.90 | 0.68 | 2.07E−04 | 3.38E−02 |
| SLBP | 999.60 | 0.71 | 2.13E−04 | 3.43E−02 |
| MTSS1 | 149.43 | 0.63 | 2.20E−04 | 3.49E−02 |
| PAX8 | 14.26 | −0.78 | 2.23E−04 | 3.50E−02 |
| FAS | 835.36 | 0.38 | 2.37E−04 | 3.68E−02 |
| DDHD2 | 210.16 | 0.55 | 2.41E−04 | 3.70E−02 |
| IL21R | 468.09 | 0.65 | 2.70E−04 | 4.09E−02 |
| PIK3C2B | 184.00 | 0.53 | 2.81E−04 | 4.20E−02 |
| C9orf16 | 472.77 | −0.47 | 2.86E−04 | 4.23E−02 |
| HIVEP1 | 216.76 | 0.60 | 2.94E−04 | 4.29E−02 |
| GPR132 | 317.39 | 0.54 | 3.24E−04 | 4.68E−02 |
| WNT5B | 16.54 | 0.66 | 3.29E−04 | 4.70E−02 |
| NDFIP2 | 742.96 | −0.46 | 3.42E−04 | 4.73E−02 |
| PLK3 | 100.05 | 0.68 | 3.42E−04 | 4.73E−02 |
| NOD2 | 82.53 | 0.76 | 3.40E−04 | 4.73E−02 |
| UBE2J1 | 306.15 | 0.55 | 3.63E−04 | 4.85E−02 |
| PNKD | 405.60 | −0.44 | 3.70E−04 | 4.85E−02 |
| NCOA5 | 178.15 | 0.65 | 3.68E−04 | 4.85E−02 |
| BATF3 | 210.02 | 0.76 | 3.66E−04 | 4.85E−02 |
| VCAM1 | 21.59 | 0.67 | 3.62E−04 | 4.85E−02 |
| EGR1 | 403.52 | 0.70 | 3.74E−04 | 4.85E−02 |
| IRF4 | 500.28 | 0.68 | 3.87E−04 | 4.98E−02 |
| EVC | 29.55 | 0.73 | 4.04E−04 | 5.14E−02 |
| RUNX2 | 409.34 | 0.60 | 4.09E−04 | 5.15E−02 |
| IL31RA | 7.71 | 0.69 | 4.16E−04 | 5.17E−02 |
| ZNRF1 | 64.38 | 0.68 | 4.19E−04 | 5.17E−02 |
| KDSR | 932.93 | 0.68 | 4.59E−04 | 5.61E−02 |
| IGFLR1 | 620.25 | −0.45 | 4.84E−04 | 5.73E−02 |
| SEPW1 | 1141.31 | −0.41 | 4.81E−04 | 5.73E−02 |
| IFIH1 | 182.04 | 0.63 | 4.91E−04 | 5.73E−02 |
| JMY | 84.20 | 0.73 | 4.87E−04 | 5.73E−02 |
| LOC100506668 | 44.96 | −0.66 | 4.78E−04 | 5.73E−02 |
| ETV6 | 228.14 | 0.51 | 5.09E−04 | 5.88E−02 |
| DENND4A | 237.26 | 0.56 | 5.32E−04 | 6.09E−02 |
| RGL4 | 144.59 | −0.54 | 5.47E−04 | 6.11E−02 |
| GLUL | 2097.26 | 0.68 | 5.48E−04 | 6.11E−02 |
| NOMO3 | 43.60 | 0.73 | 5.41E−04 | 6.11E−02 |
| CD74 | 27339.03 | 0.58 | 5.53E−04 | 6.11E−02 |
| ZDHHC3 | 298.89 | 0.44 | 5.62E−04 | 6.12E−02 |
| NOTCH2 | 853.97 | 0.53 | 5.63E−04 | 6.12E−02 |
| MAF1 | 368.42 | 0.71 | 6.22E−04 | 6.49E−02 |
| CXCL10 | 208.18 | 0.61 | 6.20E−04 | 6.49E−02 |
| MLLT3 | 398.50 | −0.36 | 6.19E−04 | 6.49E−02 |
| HMSD | 25.09 | 0.72 | 6.08E−04 | 6.49E−02 |
| ZNF704 | 8.42 | −0.72 | 6.22E−04 | 6.49E−02 |
| INSIG1 | 1252.70 | 0.67 | 6.36E−04 | 6.57E−02 |
| TACO1 | 143.99 | 0.57 | 6.61E−04 | 6.76E−02 |
| TRIM14 | 485.46 | 0.58 | 6.75E−04 | 6.76E−02 |
| TARSL2 | 56.38 | 0.69 | 6.70E−04 | 6.76E−02 |
| PON2 | 102.68 | 0.61 | 6.75E−04 | 6.76E−02 |
| RPL37A | 7903.48 | −0.32 | 7.08E−04 | 6.92E−02 |
| SLC25A10 | 110.25 | −0.56 | 7.04E−04 | 6.92E−02 |
| RGMB | 21.71 | 0.69 | 6.98E−04 | 6.92E−02 |
| TTC39C | 569.24 | −0.57 | 7.25E−04 | 7.04E−02 |
| AKIRIN1 | 412.99 | 0.66 | 7.37E−04 | 7.04E−02 |
| FAM173B | 205.91 | −0.52 | 7.32E−04 | 7.04E−02 |
| CLPTM1 | 190.39 | 0.55 | 7.58E−04 | 7.17E−02 |
| ANXA11 | 844.72 | 0.63 | 7.62E−04 | 7.17E−02 |
| FBXO32 | 92.00 | 0.63 | 7.79E−04 | 7.28E−02 |
| GET4 | 231.33 | 0.65 | 7.91E−04 | 7.34E−02 |
| RCN2 | 268.54 | 0.71 | 8.00E−04 | 7.37E−02 |
| ALDH4A1 | 60.23 | −0.60 | 8.37E−04 | 7.66E−02 |
| CD58 | 861.00 | 0.36 | 8.66E−04 | 7.70E−02 |
| LYSMD2 | 117.14 | 0.71 | 8.63E−04 | 7.70E−02 |
| NFKBIA | 2292.72 | 0.62 | 8.58E−04 | 7.70E−02 |
| MKNK1 | 417.36 | 0.46 | 8.59E−04 | 7.70E−02 |
| TMEM121 | 10.57 | 0.69 | 8.98E−04 | 7.88E−02 |
| PROSER1 | 368.14 | 0.46 | 8.96E−04 | 7.88E−02 |
| CIRBP | 919.69 | −0.48 | 9.26E−04 | 7.96E−02 |
| MTDH | 663.91 | 0.49 | 9.15E−04 | 7.96E−02 |
| PPP1CC | 799.37 | 0.48 | 9.24E−04 | 7.96E−02 |
| PIR | 64.21 | 0.65 | 9.64E−04 | 8.12E−02 |
| APOBR | 94.19 | −0.52 | 9.56E−04 | 8.12E−02 |
| B3GNT2 | 207.39 | 0.66 | 9.64E−04 | 8.12E−02 |
| DECR1 | 876.66 | −0.36 | 9.88E−04 | 8.27E−02 |
| MAP3K6 | 29.88 | 0.68 | 9.97E−04 | 8.29E−02 |
| TAF4B | 26.05 | 0.69 | 1.03E−03 | 8.47E−02 |
| PCED1B | 173.45 | −0.48 | 1.04E−03 | 8.55E−02 |
| OGFOD3 | 133.69 | 0.54 | 1.05E−03 | 8.55E−02 |
| C1orf228 | 60.18 | −0.68 | 1.19E−03 | 9.61E−02 |
| DNAJC5B | 7.46 | 0.55 | 1.20E−03 | 9.67E−02 |
| SLC25A22 | 119.57 | 0.58 | 1.21E−03 | 9.70E−02 |
| BCL2L11 | 141.49 | −0.67 | 1.25E−03 | 9.87E−02 |
| RPL21P28 | 6107.53 | −0.37 | 1.29E−03 | 9.87E−02 |
| TMOD1 | 25.25 | 0.63 | 1.29E−03 | 9.87E−02 |
| CDKN2A | 120.19 | −0.64 | 1.28E−03 | 9.87E−02 |
| LRP8 | 142.29 | 0.54 | 1.29E−03 | 9.87E−02 |
| MLLT4 | 66.25 | 0.64 | 1.27E−03 | 9.87E−02 |
| ADAP1 | 23.82 | −0.68 | 1.26E−03 | 9.87E−02 |
| JAK1 | 1257.98 | 0.52 | 1.31E−03 | 9.96E−02 |
| IF144L | 39.53 | 0.68 | 1.32E−03 | 9.96E−02 |
| MROH8 | 23.32 | −0.67 | 1.33E−03 | 9.99E−02 |

Gene Expression - CD4+
CD4+ no stimulation

| | | | | |
|---|---|---|---|---|
| JUN | 60.51 | 1.63 | 2.17E−08 | 2.99E−04 |
| GPA33 | 114.19 | −1.53 | 5.94E−08 | 4.08E−04 |
| KRT1 | 120.23 | −1.45 | 6.42E−07 | 2.95E−03 |
| EGR1 | 123.62 | 1.30 | 6.20E−06 | 2.13E−02 |
| CIITA | 333.26 | 1.22 | 9.77E−06 | 2.69E−02 |
| UBD | 34.30 | 1.12 | 2.49E−05 | 5.71E−02 |
| KLHL23 | 19.00 | 1.11 | 4.37E−05 | 7.78E−02 |
| SCD | 428.59 | 1.00 | 4.52E−05 | 7.78E−02 |
| HLA-DOA | 258.09 | 1.17 | 5.35E−05 | 8.19E−02 |
| ALPK2 | 16.74 | 1.06 | 6.57E−05 | 9.04E−02 |
| CXCL10 | 162.12 | 1.11 | 7.88E−05 | 9.85E−02 |

CD4+ 4 hours

| | | | | |
|---|---|---|---|---|
| C17orf61-PLSCR3 | 41.84 | −1.79 | 1.11E−13 | 1.35E−09 |
| ENPP2 | 97.77 | 1.77 | 4.63E−13 | 2.81E−09 |
| FILIP1L | 180.31 | 1.32 | 1.01E−08 | 4.10E−05 |
| HLA-DQA2 | 556.29 | 1.31 | 8.41E−08 | 2.55E−04 |
| UBD | 81.00 | 1.22 | 1.36E−07 | 3.30E−04 |
| CIITA | 367.08 | 1.20 | 2.75E−07 | 4.76E−04 |
| GJB2 | 308.61 | 1.25 | 2.62E−07 | 4.76E−04 |
| P2RY14 | 53.02 | 1.19 | 1.13E−06 | 1.71E−03 |
| IL4I1 | 236.25 | 0.95 | 4.75E−06 | 6.41E−03 |
| HLA-DOA | 210.18 | 1.08 | 9.61E−06 | 1.09E−02 |
| ENOX1 | 42.28 | 0.95 | 9.92E−06 | 1.09E−02 |
| HLA-DRA | 6849.48 | 1.06 | 1.21E−05 | 1.23E−02 |
| NTRK2 | 446.21 | 1.05 | 1.36E−05 | 1.27E−02 |
| HLA-DRB1 | 3546.08 | 1.01 | 2.44E−05 | 1.98E−02 |
| COL6A1 | 30.67 | 0.93 | 2.37E−05 | 1.98E−02 |
| DMD | 201.87 | 1.00 | 3.82E−05 | 2.58E−02 |
| BTN2A2 | 155.42 | 0.93 | 3.79E−05 | 2.58E−02 |
| HLA-DPB1 | 2102.74 | 0.99 | 3.61E−05 | 2.58E−02 |
| HLA-DMB | 313.07 | 0.96 | 7.63E−05 | 4.67E−02 |
| HLA-DRB5 | 1032.55 | 0.97 | 7.70E−05 | 4.67E−02 |
| HLA-DQB2 | 79.53 | 0.96 | 8.54E−05 | 4.94E−02 |
| JUN | 83.06 | 0.94 | 9.46E−05 | 5.22E−02 |
| GCSAM | 93.84 | 0.92 | 1.50E−04 | 7.59E−02 |
| HLA-DPA1 | 4787.23 | 0.93 | 1.47E−04 | 7.59E−02 |
| DDIT4 | 1468.62 | 0.88 | 1.58E−04 | 7.69E−02 |
| HLA-DRB6 | 350.45 | 0.91 | 1.88E−04 | 8.78E−02 |
| C7orf55-LUC7L2 | 28.17 | 0.81 | 1.97E−04 | 8.87E−02 |
| BCL2A1 | 446.67 | −0.87 | 2.15E−04 | 9.30E−02 |
| KRT7 | 48.31 | 0.90 | 2.36E−04 | 9.88E−02 |

CD4+ 24 hours

| | | | | |
|---|---|---|---|---|
| GJB2 | 244.40 | 1.55 | 1.49E−09 | 1.97E−05 |
| UBD | 37.13 | 1.49 | 5.44E−09 | 3.59E−05 |
| NTRK2 | 114.68 | 1.43 | 5.02E−08 | 2.21E−04 |
| THY1 | 29.23 | 1.26 | 5.32E−07 | 1.55E−03 |
| HLA-DQA2 | 518.42 | 1.28 | 5.87E−07 | 1.55E−03 |
| HLA-DRA | 6566.62 | 1.10 | 9.17E−07 | 2.02E−03 |
| G0S2 | 63.95 | 1.29 | 1.18E−06 | 2.23E−03 |
| CXCL10 | 235.90 | 1.25 | 2.43E−06 | 4.01E−03 |
| IER2 | 1658.94 | 0.92 | 2.84E−06 | 4.16E−03 |
| CIITA | 486.06 | 1.10 | 3.80E−06 | 5.01E−03 |
| DOHH | 120.11 | 1.18 | 5.65E−06 | 6.78E−03 |
| ADA | 519.36 | 1.08 | 6.44E−06 | 7.09E−03 |
| MSC | 269.09 | 1.16 | 1.02E−05 | 1.03E−02 |
| JUNB | 1127.04 | 1.01 | 1.48E−05 | 1.39E−02 |
| DMD | 131.95 | 1.14 | 1.78E−05 | 1.42E−02 |
| CDK6 | 1226.39 | 0.95 | 1.64E−05 | 1.42E−02 |
| HLA-DRB1 | 3456.58 | 1.05 | 1.83E−05 | 1.42E−02 |
| HLA-DOA | 266.72 | 1.11 | 2.16E−05 | 1.50E−02 |
| SH3BP5 | 243.81 | 1.06 | 2.11E−05 | 1.50E−02 |
| LGMN | 325.32 | −1.04 | 4.34E−05 | 2.86E−02 |
| ACSL1 | 94.77 | 1.04 | 4.77E−05 | 3.00E−02 |
| ANXA3 | 49.93 | 1.06 | 5.00E−05 | 3.00E−02 |
| HLA-DRB5 | 1038.39 | 1.02 | 5.67E−05 | 3.25E−02 |
| EMC8 | 172.54 | 1.05 | 7.30E−05 | 3.90E−02 |
| FILIP1L | 89.49 | 1.05 | 7.39E−05 | 3.90E−02 |
| PDCD1 | 177.81 | −0.95 | 7.77E−05 | 3.94E−02 |
| ANK3 | 269.28 | 0.87 | 9.09E−05 | 4.28E−02 |
| HLA-DRB6 | 344.95 | 1.01 | 8.83E−05 | 4.28E−02 |
| IFNG | 770.38 | 1.02 | 9.49E−05 | 4.32E−02 |
| MPZL1 | 230.37 | 0.96 | 1.12E−04 | 4.65E−02 |
| TMEM165 | 255.98 | 0.98 | 1.13E−04 | 4.65E−02 |
| NOD2 | 117.11 | 1.01 | 1.13E−04 | 4.65E−02 |
| DGAT2 | 26.29 | 0.93 | 1.30E−04 | 5.18E−02 |
| AKIRIN1 | 410.23 | 0.94 | 1.52E−04 | 5.47E−02 |
| ELL2 | 329.55 | 0.95 | 1.47E−04 | 5.47E−02 |
| MATN4 | 14.59 | 0.89 | 1.52E−04 | 5.47E−02 |
| SREBF2 | 321.25 | 0.86 | 1.57E−04 | 5.47E−02 |
| INSIG1 | 1187.28 | 0.88 | 1.57E−04 | 5.47E−02 |
| BATF3 | 159.07 | 0.98 | 1.67E−04 | 5.63E−02 |
| HLA-DPB1 | 2085.87 | 0.90 | 1.83E−04 | 5.84E−02 |
| MAF1 | 408.02 | 0.98 | 1.86E−04 | 5.84E−02 |
| HLA-DPA1 | 4953.28 | 0.89 | 1.81E−04 | 5.84E−02 |
| ADCY1 | 116.14 | 0.99 | 1.97E−04 | 6.06E−02 |
| NFKBIA | 2113.32 | 0.87 | 2.27E−04 | 6.82E−02 |
| JUN | 161.21 | 0.88 | 2.37E−04 | 6.89E−02 |
| P2RY14 | 42.63 | 0.97 | 2.40E−04 | 6.89E−02 |
| ANXA11 | 949.22 | 0.82 | 2.86E−04 | 7.99E−02 |
| COTL1 | 3174.02 | 0.90 | 2.91E−04 | 7.99E−02 |
| HMHA1 | 1559.34 | 0.74 | 3.03E−04 | 8.15E−02 |
| IL23R | 32.41 | 0.95 | 3.12E−04 | 8.25E−02 |
| GCSAM | 103.55 | 0.94 | 3.21E−04 | 8.30E−02 |
| ZFAND5 | 664.12 | 0.91 | 3.41E−04 | 8.66E−02 |
| IL21 | 14.28 | 0.83 | 3.87E−04 | 9.62E−02 |
| ACADVL | 1075.91 | 0.83 | 3.97E−04 | 9.69E−02 |
| IL21R | 430.22 | 0.78 | 4.18E−04 | 9.85E−02 |
| SLBP | 1001.06 | 0.84 | 4.13E−04 | 9.85E−02 |

Gene Expression - CD8+
CD8+ No Stimulation

| | | | | |
|---|---|---|---|---|
| CXCL10 | 153.97 | 2.25 | 2.17E−15 | 3.94E−11 |
| JUNB | 508.83 | 1.66 | 1.71E−12 | 1.55E−08 |
| NTRK2 | 28.44 | 1.52 | 4.05E−08 | 2.45E−04 |
| MSC | 285.77 | 1.54 | 5.87E−08 | 2.67E−04 |
| VNN2 | 133.36 | −1.29 | 5.03E−07 | 1.83E−03 |

CD8+ 4 hours

| | | | | |
|---|---|---|---|---|
| JUNB | 1517.36 | 0.97 | 3.16E−08 | 5.80E−04 |
| CXCL10 | 222.56 | 1.20 | 9.65E−08 | 8.85E−04 |
| ENOX1 | 35.18 | 1.06 | 4.02E−07 | 2.46E−03 |
| ENPP2 | 128.55 | 1.02 | 2.06E−06 | 9.43E−03 |
| DDIT4 | 1901.41 | 1.01 | 4.50E−06 | 1.65E−02 |
| NTRK2 | 202.63 | 1.02 | 5.71E−06 | 1.75E−02 |
| GCSAM | 42.18 | 0.92 | 2.56E−05 | 6.44E−02 |
| IL5 | 88.58 | −0.94 | 2.81E−05 | 6.44E−02 |

CD8 24 hours

| | | | | |
|---|---|---|---|---|
| ENPP2 | 165.05 | 1.27 | 6.54E−11 | 8.48E−07 |
| GJB2 | 308.95 | 1.03 | 7.10E−08 | 4.61E−04 |
| C4orf26 | 142.26 | 1.04 | 1.38E−07 | 5.95E−04 |
| MX1 | 271.76 | 0.95 | 5.44E−07 | 1.76E−03 |
| NTRK2 | 67.96 | 0.95 | 1.98E−06 | 5.14E−03 |
| JUNB | 980.64 | 0.84 | 3.62E−06 | 7.82E−03 |
| TNFRSF8 | 328.21 | 0.90 | 6.23E−06 | 1.16E−02 |
| DGAT2 | 30.68 | 0.76 | 7.26E−06 | 1.18E−02 |
| ELL2 | 340.54 | 0.83 | 9.24E−06 | 1.33E−02 |
| IL4I1 | 106.36 | 0.85 | 1.26E−05 | 1.63E−02 |
| ITPR1 | 809.06 | 0.71 | 2.25E−05 | 2.62E−02 |
| HLA-DRB6 | 370.24 | 0.80 | 2.43E−05 | 2.62E−02 |
| GCSAM | 64.54 | 0.74 | 6.71E−05 | 6.70E−02 |
| ADCY1 | 62.65 | 0.76 | 7.58E−05 | 7.02E−02 |
| HLA-DQA2 | 542.96 | 0.77 | 8.19E−05 | 7.08E−02 |
| HLA-DRA | 7408.60 | 0.67 | 1.12E−04 | 9.08E−02 |
| ANK3 | 189.91 | 0.76 | 1.29E−04 | 9.86E−02 |

TABLE 8
Selected Overlap Results Table of select enriched gene sets in MSigDB with computed
overlaps of DE genes upregulated in BBζ compared to 28ζ CARs in CD4+ and CD8+ samples combined.
‘k’ refers to the number of genes in the intersection of the query set with a set from MSigDB. ‘K’ refers
to the number of genes in the set from MSigDB. ‘FDR q-value’ is false discovery rate analog
of hypergeometric using Benjamini and Hochberg to correct for multiple testing.

| | # Genes | | | FDR |
|---|---|---|---|---|
| Gene Set Name | in Overlap | k/K | p-value | q-value |
| GO_RESPONSE_TO_CYTOKINE | 22 | 0.0308 | 1. 01E−21 | 1.69E−17 |
| GO_INTERFERON_GAMMA_MEDIATED_SIGNALING_PATHWAY | 11 | 0.1571 | 3.74E−19 | 1.25E−15 |
| GO_IMMUNE_SYSTEM_PROCESS | 27 | 0.0136 | 1.37E−17 | 3.83E−14 |
| GO_POSITIVE_REGULATION_OF_CELL_CELL_ADHESION | 13 | 0.0535 | 3.54E−16 | 5.32E−13 |
| GO_MHC_CLASS II_PROTEIN_COMPLEX | 7 | 0.4375 | 3.82E−16 | 5.32E−13 |
| GO_REGULATION_OF_CELL_ACTIVATION | 15 | 0.031 | 5.10E−15 | 5.34E−12 |
| GO_LUMENAL_SIDE_OF_MEMBRANE | 7 | 0.2121 | 1.39E−13 | 9.71E−11 |
| KEGG_ALLOGRAFT_REJECTION | 7 | 0.1842 | 4.08E−13 | 2.63E−10 |
| GO_CLATHRIN_COATED_VESICLE_MEMBRANE | 8 | 0.0988 | 1.54E−12 | 8.31E−10 |
| GO_TRANS_GOLGI_NETWORK_MEMBRANE | 8 | 0.0988 | 1.54E−12 | 8.31E−10 |
| REACTOME_PHOSPHORYLATION_OF_CD3_AND_TCR_ZETA_CHAINS | 5 | 0.3125 | 5.68E−11 | 1.53E−08 |
| GO_REGULATION_OF_CELL_PROLIFERATION | 18 | 0.012 | 6.38E−11 | 1.70E−08 |
| HALLMARK_TNFA_SIGNALING_VIA_NFKB | 9 | 0.045 | 7.39E−11 | 1.77E−08 |

### Example 7—Antigen Stimulation of CARs Bearing the 4-1BB Vs. CD28 Co-Stimulation Domains Respectively Induces a T_{H}1 vs. T_{H}2 Polarization Program in CD4 T Cells

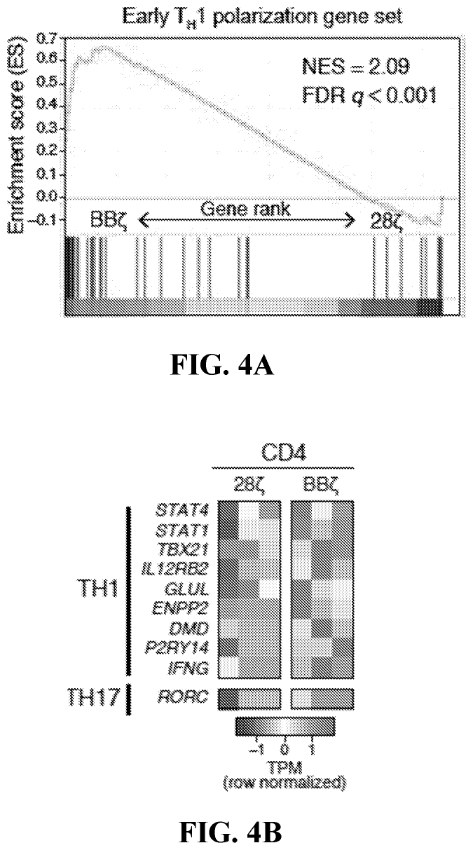

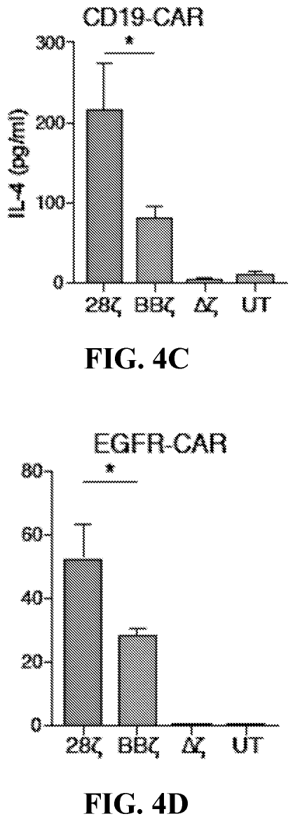

T_{H}1 polarizing genes were enhanced in CD4⁺ BBζ CAR T cells after antigen stimulation and at rest (FIGS. 4A and 10E), whereas CAR activated 28ζ cells were enriched for T_{H}2 early polarizing genes²⁸ (differentially expressed genes up in 28ζ with FDR<0.1, GSEA: p-val=9.12e-9, FDR=4.44e-5). In particular, IL12RB2, which encodes a subunit of the IL12 receptor and is known to be upregulated in T_{H}1 polarized cells^{5,29}, was up-regulated in both 28ζ and BBζ cells at the 4 hour time point after antigen stimulation, but only BBζ cells maintained higher expression at 24 hours (FIG. 10B). Other genes induced in CD4⁺ BBζ CAR T cells included the T_{H}1 transcription factor genes EGR1 and TBX21 and the T_{H}17 transcription factor gene RORC (FIG. 4B). Consistently CAR-activated 28ζ CAR T cells (both CD19-CAR and EGFR-CAR) secreted higher levels of the T_{H}2 cytokine IL4 and of IL-5 than BBζ CAR T cells (FIGS. 4C-4D, FIG. 10F). Thus, the type of co-stimulatory domain embedded in the CAR plays a strong role in the cytokine polarization profile of CAR T cells, particularly after antigen stimulation.

### Example 8—Stimulation Activates a Range of Cellular Programs in Different CAR T Cells

To examine the spectrum of activated CAR T cell populations that underlies these global distinctions, we turned to the scRNA-Seq profiles (FIG. 5A), observing a separation of BBζ CAR T cells, whereas profiles of 28ζ and ζ containing CAR T cells were similarly distributed (FIG. 5A). Gene programs underlying these cellular distributions were identified with a latent Dirichlet allocation (LDA), or “topic modeling”, approach. This unsupervised approach has recently been successfully applied for the analysis of profiles of single immune cells³⁰, capturing the continuous nature of their variation, and the fact that different T cell subsets can activate similar programs. For instance, a CD4 T cell can have an effector memory program, a Th1 program, and a Th17 program during a particular antigen response³¹. Next, a network analysis was performed (Methods-Example 11) to associate programs with putative transcription factor (TF) regulators.

The programs reflected main facets of T cell biology and varied between the different stimulated CAR T cells (FIG. 5B and FIG. 11). For example, A T_{H}2 program (topic 8) scored highly in a small subset of ζ and 28ζ CAR T cells, suggesting this subset accounts for their bulk Th2 response; the program was associated with ETS1, a known regulator of T_{H}2 cytokines³². A polyfunctional program of multiple cytokines and chemokines, including IFNγ, IL-3, and IL-9 (topic 14) was expressed in cells with high activation signatures (FIGS. 5C-5D, Table 5), and associated with NFκB and NFAT TFs. A cell proliferation program (topic 12), was mutually exclusive with the cytokine secreting cells and associated with many known regulators of proliferation (Table 10). A CD8 specific program (topic 9) was clearly expressed in the CD8A expressing cells (FIG. 5E) and a T_{H}17-like program including IL17RB, IL17F, and IL22 as well as IL10 and associated with STAT1, STAT3 and SMAD1 as it regulators.

TABLE 10
Transcriptional regulators of the top 100 genes in topic 12 discovered by netork
analysis (methods). Overlap - number of genes regulated in topic. Set size -
number of genes known to be regulated by the TF Jaccard - Jaccard similarity index

| Gene | pvalue | FDR | overlap | set size | jaccard |
|---|---|---|---|---|---|
| E2F1 | 2.3168E−25 | 5.6864E−21 | 54 | 2708 | 0.00209205 |
| E2F4 | 4.6904E−19 | 5.7561E−15 | 35 | 1345 | 0.00143044 |
| ORC2 | 1.494E−14 | 7.3337E−11 | 8 | 21 | 0.00034526 |
| MCM6 | 1.5964E−13 | 3.5621E−10 | 8 | 27 | 0.00034517 |
| NFYA | 4.6662E−13 | 9.544E−10 | 36 | 2242 | 0.00141933 |
| MCM7 | 2.6818E−12 | 4.7016E−09 | 8 | 37 | 0.00034502 |
| MCM2 | 3.3852E−12 | 5.54E−09 | 8 | 38 | 0.00034501 |
| E2F3 | 3.0641E−11 | 4.4239E−08 | 24 | 1119 | 0.00098957 |
| E2F2 | 1.042E−10 | 1.2787E−07 | 25 | 1296 | 0.00102337 |
| E2F6 | 9.9447E−10 | 1.017E−06 | 22 | 1107 | 0.00090748 |
| MCM3 | 1.3548E−09 | 1.3301E−06 | 6 | 27 | 0.00025885 |
| BRCA1 | 2.4612E−09 | 2.1574E−06 | 9 | 122 | 0.00038675 |
| E2F5 | 5.3715E−09 | 4.3946E−06 | 21 | 1102 | 0.00086637 |
| E2F7 | 2.6376E−08 | 1.904E−05 | 20 | 1094 | 0.00082535 |
| RFC1 | 3.971E−07 | 0.00022151 | 4 | 15 | 0.00017264 |
| ATM | 7.1634E−07 | 0.00037408 | 5 | 40 | 0.00021558 |
| MCM5 | 1.113E−06 | 0.00056913 | 4 | 19 | 0.00017261 |
| MCM4 | 1.7075E−06 | 0.00082175 | 4 | 21 | 0.0001726 |
| RB1 | 2.6788E−06 | 0.00126439 | 7 | 144 | 0.00030049 |
| MYC | 3.3122E−06 | 0.00153388 | 41 | 4921 | 0.0014623 |
| hsa-miR-146a | 3.9886E−06 | 0.00177992 | 8 | 217 | 0.00034236 |
| SP1 | 4.1831E−06 | 0.00179669 | 36 | 4046 | 0.00132509 |
| RAD51 | 6.6025E−06 | 0.00257223 | 4 | 29 | 0.00017254 |
| TP53 | 1.7354E−05 | 0.00553172 | 16 | 1116 | 0.00065958 |
| RBL1 | 2.4515E−05 | 0.00761635 | 4 | 40 | 0.00017246 |
| CHAF1A | 3.2225E−05 | 0.00964538 | 3 | 15 | 0.00012948 |
| SERTAD2 | 0.00010577 | 0.0251979 | 2 | 4 | 8.6356E−05 |
| POLR2A | 0.00010736 | 0.0251979 | 4 | 58 | 0.00017232 |
| BRCA2 | 0.00015796 | 0.03400937 | 3 | 25 | 0.00012942 |
| BARD1 | 0.00015796 | 0.03400937 | 3 | 25 | 0.00012942 |
| hsa-miR-126 | 0.00021112 | 0.04318166 | 4 | 69 | 0.00017224 |
| RBL2 | 0.00047411 | 0.07809693 | 3 | 36 | 0.00012936 |

### Example 9—Antigen Specific Activation of 4-1BB CAR T Cells Induces a Distinct Program Shared in CD4 and CD8 Cells

Topic 11 scored highly specifically in BB CAR T cells (FIG. 5B), with many of its top scoring genes (FIG. 5B, Table 9) differentially expressed between BBζ and 28 ζ activated CAR T cells. These include HLA-II genes, CCR7, ENPP2, and the transcription factor BATF3 (Table 9), and TRAF1, which propagates signaling downstream of TNF receptors such as 4-1BB and is thought to directly bind to 4-1BB intracellular domain during signaling, resulting in a positive feedback loop³³. Network analysis predicted NFκB TFs and TNFAIP3 (A20)^{34,35}, as the regulators of topic 11 (FIGS. 5C and 5F).

Finally, to determine if the genes induced specifically by 4-1BB containing CARs were also induced after endogenous 4-1BB signaling³⁶, we defined a 4-1BB gene signature from our data and scored it in UT cells activated with 4-1BB ligand (41BBL). Specifically, the signature was defined by the top genes in topic 11 that were also differentially expressed in at least one time point between BBζ and 28ζ CAR T cells (Methods-Example 11). We generated a K562 cell line expressing a membrane bound anti-CD3 with or without 4-1BBL expression. UT T cells were expanded as before (FIG. 1B) with anti-CD3/CD28 beads for 7 days, rested them for 7 days, activated them for 24 hours with irradiated K562-anti-CD3 or K562-anti-CD3+4-1BBL, sorted T cells, and measured the 4-1BB signature expression (with nCounter, Methods-Example 11).

Although many of the genes in the 4-1BB signature were induced by endogenous 4-1BB signaling, a subset (FIG. 5G) was unique to stimulated CAR-T cells, including all the MHC II genes and ENPP2. Without being bound by theory, it is thought that 4-1BB signaling from the artificial CAR molecule is strong enough to induce programs that would otherwise not be induced in T cells, such as the MHC class II molecules known to be upregulated after DC maturation using 4-1BB-4-1BBL stimulation^{37,38}.

TABLE 9
Top 50 features (genes) contributing to each topic fit by a Latent
Dirichlet Allocation (LDA) topic model for K = 16, tol = 0.1
on CAR stimulated samples that included all genes

| topic_1 | topic_2 | topic_3 | topic_4 |
|---|---|---|---|
| HSPE1 | TMSB4X | ACTB | HIST1H1D |
| RPS12 | B2M | TMSB4X | HMGB2 |
| RPS8 | RPS23 | IL32 | HIST1H4C |
| RPS23 | RPS7 | CFL1 | HIST1H1B |
| YBX1 | RPS27A | PFN1 | PHLDA3 |
| STMN1 | RPS6 | MT-CO2 | HIST1H1E |
| FTL | RPL21 | MYL6 | RPL13 |
| NCL | RPL14 | LGALS1 | ACTB |
| CDK4 | RPS18 | ACTG1 | UBE2C |
| TMSB10 | RPS14 | CORO1A | KIAA0101 |
| RPS11 | RPL10 | ARHGMB | HIST1H1C |
| RPLP2 | RPS4X | MALAT1 | MALAT1 |
| HSP90AA1 | RPL18 | ALOX5AP | RPS6 |
| C1QBP | RPL10A | MT-CO3 | RPL10 |
| CCDC85B | RPL6 | GAPDH | HMGN2 |
| EBNA1BP2 | RPL11 | TMSB10 | TUBB |
| NME1 | EEF1A1 | ARPC1B | RPS7 |
| RPS18 | TMSB10 | RGL4 | FDXR |
| B2M | RPL15 | CD52 | RPS4X |
| RPL19 | RPL27A | MT-CYB | RPL15 |
| TUFM | RPL27 | FTL | ENO1 |
| SNU13 | RPL26 | RPS2 | RPL5 |
| RANBP1 | ACTG1 | VIM | TMEM106C |
| RPS4X | RPL19 | MYL12A | BIRC5 |
| TUBA1B | NPM1 | COTL1 | CKS1B |
| ODC1 | RPL24 | B2M | CENPA |
| NPM3 | RPL31 | IL7R | GAPDH |
| SNRPD1 | RPL35A | RPS12 | RPL11 |
| NOP16 | RPS25 | LY6E | SAC3D1 |
| RPL41 | RPL5 | CD7 | CENPM |
| CYCS | RPL18A | CD74 | TMSB10 |
| HIST1H4C | RPL12 | KLF2 | HIST2H2AC |
| RPL18A | EEF1B2 | PSMB9 | RPL14 |
| ZWINT | GNB2L1 | CAPZB | MYBL2 |
| RPL35A | RPS3A | PGK1 | RPS3A |
| MRPL4 | FTL | TRAC | BAX |
| NOLC1 | RPL4 | ARPC3 | RPS27A |
| RPL10 | RPS15A | RPLP1 | RPS25 |
| ACTB | NACA | LCK | H2AFX |
| C19orf48 | RPL13A | GMFG | FTL |
| NPM1 | RPS16 | RPL9 | RPS15A |
| NDUFAB1 | RPS24 | HLA-B | EEF1B2 |
| TOMM22 | RPL34 | LSP1 | TPX2 |
| RCC1 | RPL7 | HMGN2 | MKI67 |
| MYBL2 | TIMP1 | RPS15 | CENPW |
| TOMM40 | RPS5 | DDIT4 | RPS24 |
| MT-CO2 | RPS8 | FLOT1 | STMN1 |
| SNRPE | IL32 | C12orf75 | RPL26 |
| PDCD5 | RPS13 | RPL21 | YBX1 |
| SRM | RPS11 | H2AFZ | RPL7 |
| topic_5 | topic_6 | topic_7 | topic_8 |
| MT-CO1 | MT-CO1 | FTL | CCL1 |
| MT-CO2 | MT-CO2 | EIF5A | CD52 |
| MT-CO3 | MT-CO3 | GNLY | LAIR2 |
| MT-ND4 | MT-ND2 | IL32 | RPS2 |
| RPS2 | HSP90AB1 | XIST | TMSB4X |
| MT-CYB | HSP90AA1 | MT-CO3 | SH2D1A |
| MT-ATP6 | MT-ND4 | MALAT1 | CSF2 |
| TMSB4X | MALAT1 | FTH1 | AHI1 |
| TMSB10 | LDHB | TXN | JAML |
| RPL36 | MT-ND3 | LGMN | PRKCDBP |
| MT-ND2 | MT-CYB | MT-ATP6 | EIF1 |
| RPL41 | UBC | RPS4X | SEC61B |
| B2M | MT-ATP6 | NDUFA12 | GATA3 |
| BATF3 | TUBB | TRBC1 | C1orf228 |
| MT-ND3 | B2M | RPS19 | TNFRSF18 |
| RPL29 | ENO1 | EEF1A1 | CALM3 |
| CCL5 | XRCC6 | S100A11 | FABP5 |
| RPS6 | PDIA6 | RPS29 | B2M |
| RPS3 | CCT8 | ARPC1B | SUPT4H1 |
| CCDC85B | TUBB4B | PIM2 | SMS |
| RPL12 | UBE2C | TRBC2 | COX7A2 |
| LGALS1 | HNRNPM | TMSB10 | IL2RA |
| HMGB1 | HLA-C | ITM2A | IL31 |
| HIST1H4C | COX6A1 | AC133644.2 | NEK6 |
| RPL11 | MT-ND1 | RPL13 | NKG7 |
| RPS28 | RPN2 | SUB1 | SH3BGRL3 |
| RPS15A | XRCC5 | HSP90AA1 | UNQ6494 |
| ALDOA | ATP5A1 | RPS7 | APRT |
| TUBA1B | CANX | PSMA7 | ZFAS1 |
| H2AFZ | HNRNPA2B1 | RNF213 | EGR2 |
| RPL21 | TUFM | RPS18 | LAG3 |
| RPL26 | HNRNPK | PFN1 | RBPJ |
| GAPDH | CCT3 | IL2RA | UCP2 |
| GUK1 | RRM1 | PTP4A2 | UBE2F |
| SRM | KPNA2 | SMAP2 | HMGB1 |
| NKG7 | MCM7 | NBEAL1 | MDFIC |
| TMA7 | PGK1 | B2M | CIRBP |
| RPS25 | SRGN | MT-ND3 | HOPX |
| RPL9 | RPS25 | UFD1L | TNFAIP8 |
| MALAT1 | RPL27A | RPS27 | DUSP4 |
| HMGN2 | HAT1 | CXCL10 | XCL1 |
| RPL8 | RPL15 | C4orf26 | DAD1 |
| PPP1R14B | PPIB | FAM107B | GATA3-AS1 |
| RPL18A | GDI2 | ACTB | ERN1 |
| NDUFB7 | NDUFA9 | RPLP0 | IL13 |
| MT-ND1 | CCT5 | GZMH | SF3B5 |
| PSMA7 | HLA-A | UCP2 | RPL28 |
| CTDNEP1 | STIP1 | HLA-DRA | LY6E |
| RPL37A | LDHA | MIR4435-2HG | C7orf73 |
| RPS27A | HSPA9 | RPL18A | IL4 |
| topic_9 | topic_10 | topic_11 | topic_12 |
| GZMB | GZMA | BATF3 | KIAA0101 |
| NKG7 | LTB | CD74 | DUT |
| GAPDH | B2M | DDIT4 | TK1 |
| CD27 | CD52 | ARID5A | PCNA |
| CD8B | S100A4 | HLA-DRA | TYMS |
| CD8A | IL32 | G0S2 | SIVA1 |
| JUNB | S100A6 | JUNB | HELLS |
| HOPX | MALAT1 | HLA-DPA1 | CLSPN |
| CTSW | CYBA | MALAT1 | ATAD2 |
| LAG3 | TPT1 | SMAP2 | FAM111B |
| LGALS1 | EMP3 | SQRDL | CENPU |
| CCL5 | LGALS3 | ELL2 | SLBP |
| RPS3A | FTL | MIR155HG | USP1 |
| FUT7 | LST1 | HLA-DPB1 | SVIP |
| RPS5 | SH3BGRL3 | HLA-DQA1 | DNMT1 |
| XCL1 | NCR3 | CFLAR | MCM7 |
| RPS18 | TMSB4X | HLA-DRB1 | STRA13 |
| S100A4 | HCST | MTHFD2 | GINS2 |
| CSF2 | CXCR6 | ADCY1 | RRM2 |
| AIF1 | ITM2B | TNFRSF8 | FEN1 |
| MLLT11 | ALOX5AP | RYBP | ZWINT |
| IL26 | ANXA1 | KCNMA1 | DNAJC9 |
| HLA-C | FXYD5 | BIRC3 | TCF19 |
| BCAT1 | SDF2L1 | ENPP2 | CENPM |
| YBX3 | CTSC | TRAF1 | UBE2T |
| COTL1 | RPS27 | CCR7 | MYBL2 |
| FABP5 | ARL4C | CXCL10 | TMEM106C |
| RP11-291B21.2 | CD3D | HLA-DMA | CARHSP1 |
| PKM | TRAC | HMSD | RBBP8 |
| VIM | STK17B | TNIP1 | E2F1 |
| RP5-1028K7.2 | PDLIM2 | SH3BP5 | CDT1 |
| HAVCR2 | SLFN5 | ZC3H12D | MCM3 |
| LDHA | GRAP2 | H3F3B | SNRNP25 |
| UCHL1 | MT-CO2 | SATB1 | RFC2 |
| TMSB10 | ISG20 | PRKCDBP | ESCO2 |
| TRDC | XBP1 | HLA-DQB1 | ASF1B |
| CLEC2B | EVL | PALLD | CDCA5 |
| RPL7 | CD48 | KCNN4 | RFC4 |
| KLRC1 | ATP6V1G1 | IL4I1 | CDC45 |
| RPS4Y1 | BIN2 | TAGLN2 | PKMYT1 |
| FLOT1 | GBP5 | EIF1 | FBXO5 |
| CAPG | TC2N | RPL22L1 | HIST1H1D |
| SYNGR1 | ISG15 | SYNGR2 | DHFR |
| HSPA5 | IL4R | MYO1G | RNASEH2A |
| RPS15A | SAMHD1 | RAB9A | CCNE2 |
| DUSP4 | RABAC1 | RFTN1 | ATAD5 |
| HLA-A | IFITM2 | TNIP2 | CDC6 |
| BCAS4 | CTSW | ANK3 | RAD51 |
| CCDC102A | RPL27A | PLGRKT | MCM5 |
| PRKCDBP | IRF1 | PPA1 | DONSON |
| topic_13 | topic_14 | topic_15 | topic_16 |
| HSPA8 | CCL3 | MT2A | IL10 |
| NCL | CCL4 | HMGA1 | AC133644.2 |
| DDX21 | IL3 | IL7R | LGALS3 |
| SLC3A2 | IFNG | GYPC | PIM2 |
| SLC1A5 | IL9 | CORO1B | IL17RB |
| EIF3A | CCL1 | MT1E | TNFRSF4 |
| DDX5 | GZMB | LEF1 | CD6 |
| NOP56 | CCL4L2 | CCDC109B | MIIP |
| RSL24D1 | CCL3L3 | JAML | AC017002.1 |
| NOP16 | CCL5 | GCHFR | RNF213 |
| SNHG7 | CSF2 | FKBP5 | IL22 |
| CDC20 | GNLY | CD27 | IL17F |
| MYC | CXCL8 | CD7 | IKZF3 |
| NOLC1 | C10orf128 | SPINT2 | SPP1 |
| WDR43 | TNFSF14 | CHD7 | MAF |
| TAF1D | LBH | OCIAD2 | GALM |
| SARS | SLA | IDH2 | TNFRSF18 |
| NARS | IL31 | TMEM243 | PRDM1 |
| RSL1D1 | CRTAM | TLK1 | LGMN |
| PRPF38B | PHLDA1 | C12orf57 | MIR4435-2HG |
| ESF1 | LIF | C1orf228 | CTSH |
| DDX24 | SIAH2 | TNFSF13B | FDXR |
| YARS | MIR155HG | MARCKSL1 | TRAT1 |
| RRS1 | RGCC | PLAC8 | MDM2 |
| PUM3 | IER3 | RILPL2 | PHLDA3 |
| ATF4 | AC069363.1 | IFITM2 | FOXP3 |
| HSPA5 | BTG3 | IFITM3 | CD59 |
| SLC38A1 | ZBED2 | CASP8AP2 | CTLA4 |
| FNBP4 | RPS27A | PTPN6 | CDKN2A |
| SHMT2 | XCL1 | CNPY3 | PYHIN1 |
| BOP1 | RPS28 | NUCB2 | CD28 |
| ZFAS1 | RPS2 | HILPDA | CD5 |
| EIF4A2 | PRKCDBP | IRF4 | NMB |
| RRP1 | MAF | RGCC | FUT7 |
| DDX10 | NKG7 | BRD3 | TIMP1 |
| DKC1 | AC133644.2 | TPRG1 | ITM2C |
| WDR3 | RPL28 | TBC1D4 | NPDC1 |
| IARS | NPM1 | HRH2 | LEPROT |
| SNW1 | HOPX | MGST3 | PHTF2 |
| CD3EAP | NFKBIA | PHACTR2 | CASP1 |
| NOC3L | RPL23A | CCDC28B | CDKN1A |
| PES1 | SLC1A5 | MKKS | F2R |
| DHX36 | ODC1 | TESPA1 | ITGB1 |
| U2SURP | GAPDH | GPANK1 | PMVK |
| TIMM44 | RPL12 | SESN3 | TSPAN17 |
| C6orf48 | IL13 | LIF | PDE4B |
| DNAJA2 | ZEB2 | TMIGD2 | IGLC7 |
| GTPBP4 | RGS16 | N4BP2 | CHDH |
| SNHG12 | TNFAIP8 | MCM6 | ARID5B |
| TOP1 | CYCS | GNPDA1 | TM7SF3 |

### Example 10—Discussion

Examples 1-9 generated a transcriptional atlas of first- and second-generation human CD19-specific CAR T cells before and after signaling through their CAR. Previous studies have not assessed the transcriptional effects of driving constitutive CAR expression in resting human T cells. In at least the working examples herein, a transcriptional signature in functional resting CAR-modified T cells is identified, indicating the presence of ligand-independent transcriptional activity from the CD3C chain in CAR T cells. These transcriptional differences are subtler than the ligand-independent effects that have been described in c-Met and GD2-directed CAR T cells^{23,24}, since the presence of those CARs in T cells induced ligand-independent cytokine secretion and proliferation, whereas the CD19 CAR T cells were used do not. EGFR CARs replicated the tonic signaling signature suggesting it can be used as a screening tool to determine the degree of tonic CD3ζ activity.

A transcriptional effect of 4-1BB in resting CAR T cells both at the population and the single cell level was identified. The level of 4-1BB tonic signaling in CAR T cells can affect the heterogeneity and functional state even before the cells are administered to a patient.

Profiling of stimulated CAR T cells showed that while the same genes are upregulated following TCR and CAR activation, TCR signaling induces far stronger induction, for the whole population and at the cell intrinsic level. This is consistent with the poorer organization of the supramolecular activation cluster (SMAC) and the fewer ITAMs of the CD3C chain in CARs vs. a full TCR complex³⁹.

Comparing stimulated BBζ vs. 28ζ CAR T cells, they induced T_{H}1 vs. T_{H}2 polarizing genes, respectively, the former reflecting a shift across the entire cell population, but the latter reflecting a minor subset at the single cell level. The difference in cytokine profiles based on the CAR's co-stimulatory domain may help refine the treatment of cytokine release syndrome based on the CAR product causing the syndrome. Furthermore, T_{H}1 CD4⁺ T cells are known to be important for CD8⁺ T cell activity^{40,41}.

A unique gene program induced after signaling through 4-1BB in CAR T cells was recovered, including MHC class II genes, the TFs BATF3 and JUNB, as well as BIRC3, ENPP2, CXCL10 and CCR7. In addition to the predicted regulators of the NFκB family members and A20, other TFs may regulate the program, including its own members BATF3 and JUNB. Tonic signaling from NFκB has been found to be critical for both T and B cell longevity^{42,43}. Tonic signaling from the 4-1BB co-stimulation domain in CAR T cells may similarly promotes the increased persistence seen in BBζ CAR T cells.

Other aspects of these CAR T cells that could affect their phenotypes in patients include differences in cytokines and cytokine receptors. For example, IL21 and IL21R were observed to be upregulated in BBζ CAR T cells. IL-21 secretion from CD4⁺ cells is known to support the formation of memory CD8⁺ T cells, which can be important in the production of a lasting anti-tumor CD8⁺ immune response and may further explain the increased persistence of BBζ CARs⁴⁴. IL12RB2, which encodes a subunit of the IL12 receptor, was significantly upregulated in BBζ CARs. IL-12-secreting CARs with CD28 co-stimulatory domains are being developed by various groups, but these data suggest that BBζ CARs may be more sensitive to the additional IL-12⁴¹.

Many, but not all, of the genes in the demonstrated CAR 4-1BB program were also unregulated following endogenous 4-1BB signaling in UT T cells; a subset was only induced when 4-1BB is activated in the context of a CAR co-stimulatory domain. This highlights the importance of studying these costimulatory programs in the context of a CAR construct, which may induce additional signaling pathways that provide additional functions to BBζ CAR T cells, such as antigen presentation through MHC class II.

Together, these data expand our understanding of how novel antigen receptors affects the gene expression profile, functional state, and ultimately, fate of engineered human T cells. They highlight unexpected differences between endogenous TCR and 4-1BB signaling compared to the effect of each module in a CAR. This can enhance CAR therapies by providing greater insight into the selection and engineering of costimulatory domains for specific cancers.

### Example 11—Methods for Examples 1-10

### Generation of CAR Constructs and CAR T Cells

Together, these data expand our understanding of how novel antigen receptors affects the gene expression CD19 and EGFR-specific CARs were synthesized and cloned into a third-generation lentiviral plasmid backbone under the regulation of a human EF-1α promoter (GenScript USA Inc). Replication-defective lentiviral vectors were produced by four plasmids co-transfected into HEK293T cells using TransIT-2020 transfection reagent (Mirus). Supernatants were collected 24h and 48h after transfection and filtered. Virus was concentrated by ultracentrifugation. Vector was harvested and stored at −80C. Healthy donor leukopaks were obtained from the Blood Transfusion Services at Massachusetts General Hospital under an IRB-approved protocol to obtain discarded tissues. CD4⁺ and CD8⁺ T cells were negatively selected using RosetteSep Kits with a Ficoll gradient (Stemcell Technologies). For the bulk RNAseq experiments, enriched T cells were further purified by CD3+CD4+ or CD3+CD8+ using fluorescence-activated cell sorting (FACS) with a BD FACSAria III (BD Biosciences). CD4⁺ and CD8⁺ T cells were mixed at a 1:1 ratio prior to expansion. For single cell experiments T cells were used at the donor specific CD4:CD8 ratio.

### Target Cell Lines

The human embryonic kidney cell line 293 (HEK392T) and Nalm6 cell lines were purchased from American Tissue Culture Collection (ATCC). Both cell lines were expanded in RPMI-1640 with 1× L-GlutaMAX and 25 mM HEPES (Gibco, Life Technologies) and supplemented with 10% heat-inactivated fetal bovine serum (FBS, Gibco Life Technologies). U87 cell line was from ATCC and expanded in EMEM with 10% FBS. U87 cells were passaged with 0.05% trypsin (Gibco, Life Technologies). Target cells were irradiated with 10 000 rads and frozen in FBS with 10% DMSO to be thawed prior to stimulation of CAR T cells. K562 cells were transduced to express anti-CD3 scFv and 4-1BBL as described previously i.

### T Cell In Vitro Expansion and Transduction for RNA-Seq

Together T cells were plated in a 24 well plate at 1 million cells/ml in RPMI-1640 with 1× L-GlutaMAX and 25 mM HEPES (Gibco, Life Technologies) supplemented with 10% FBS and IL-2 (20 IU/ml-Peprotech). Anti-CD3/CD28 beads (Dynabeads, Invitrogen) were added with a 3:1 bead-to-cell ratio. T cells were cultured for one day and then transduced with one of the four lentiviral constructs at a MOI of 5. Cells were counted and maintained at 5e⁵/ml with IL-2 replaced every 2 days during bead expansion and resting period. Beads were removed on day 7 using a magnet and cells were rested for a further 7 days. T cells were checked for mCherry expression and purity by flow cytometry analysis on day 13. On day 14, each CAR T cell population was divided into 5 wells at 5e⁵ CAR T cells/ml in 6 well plate to be either left unstimulated or stimulated with Nalm6 or anti-CD3/CD28 beads (1:1 T cells-to-Target/Bead ratio) for 4 or 24 hours prior to staining and sorting. Cells were stained with CD3-FITC (UCHT1-BioLegend), CD4-BV786 (SK3, BD Biosciences), CD8-APC-H7 (SK1, BD Pharmingen) and CD69-APC (FN50, BioLedgend). DAPI was added before FACS. 5 000 cells were sorted in technical duplicates on CD3+, CD4+ or CD8+ and mCherry⁺ with a MoFlo Astrios EQ cell sorter (Beckman Coulter) and resuspended in lysis buffer to be bulk sequenced at the Broad institute.

For single cell RNA sequencing samples were generated in the same way as described for bulk except that the Δζ construct was not included in the experiment and cells were either left unstimulated or stimulated for 24 hours with irradiated Nalm6 cells prior to sorting for scRNAseq. In addition to Nalm6 cell stimulation, the UTD sample was also stimulated for 24 hours with K563 cells expressing anti-CD3 scFv. Following stimulation T cells were sorted on a CD4⁺ or CD8⁺ gate and mCherry⁺. Single cell sequencing was repeated with cells from two normal human donors.

### Flow Cytometry

All antibodies were purchased from BioLegend unless otherwise stated. The following antibodies were used: HLA DR-PacBlue (clone L243), CD4-FITC (clone OKT4), CD8-APC-H7 (clone SK1, BD Pharmingen), PD1-BV711 (clone EH122H7). Cells were stained for 30 minutes in the dark at 4° C. and washed twice in PBS with 2% FBS. We added DAPI for cell viability directly before running flow. We collected events on a Fortessa x-20 (Becton Dickinson) and analyzed the data with FlowJo software (Tree Star).

### Bulk RNA-Seq

CAR T cells were collected using a FACS machine, resuspended at 200 cell/l in lysis buffer comprised of TCL buffer (QIAGEN 1031576) plus 1% 2-mercaptoethanol (Sigma 63689) and immediately frozen at −80° C.

For preparation of RNA-Seq libraries, cells were thawed (1,000 cells per sample) and purified with 2.2× RNAClean SPRI beads (Beckman Coulter Genomics) without a final elution². RNA capture beads were air-dried and processed immediately for cDNA synthesis. SMART-Seq2 protocol was carried out as previously described³ with minor modifications in the reverse transcription step⁴. Each PCR was performed in a 25 μl reaction with 15 cycles for cDNA amplification. We used 0.25 ng cDNA of each sample and one-fourth of the standard Illumina NexteraXT reaction volume in both the fragmentation and final PCR amplification steps. Up to 30 libraries were pooled per one Illumina NextSeq run (˜500 million reads), and sequenced 50×25 paired-end reads using a single kit on the NextSeq500 5 instrument.

### Bulk RNA-Seq Initial Read Alignment and QC

BAM files from bulk RNA-Seq were converted to merged, demultiplexed FASTQ files. Reads were mapped to the UCSC hg19 human transcriptome using Bowtie⁵, and transcript-per-million (TPM) values were calculated with RSEM v1.2.8 in paired-end mode⁶.

Samples passed QC if the number of aligned reads greater than 10⁷, the percent of reads mapped above 30% and the percent rRNA was in the range of 10-30%. We calculated the correlation between technical duplicates and further interrogated any duplicates with an r²<0.9. Duplicates were then averaged for downstream analysis.

### Raw Data “Scrubbing” for Removal of Donor Specific SNPs

Removal of donor specific SNPs was required by the IRB to de-identify the sequencing information from anonymous human donors, since we obtained discarded tissues under an IRB-approved protocol. Both single cell and bulk RNA-Seq reads were scrubbed to maintain gene expression levels whilst simultaneously removing any donor specific SNPs before uploading to the uploading to the SRA database, accession number PRJNA554339. To this end, fastq files were aligned with STAR aligner⁷ to output a BAM file of aligned reads and a splice junction file. Splice junctions were then added to the reference genome for each sample and then realigned with STAR resulting in a BAM file with splice aware alignment.

The file was used for variant calling using the Genome Analysis Tool Kit (GATK)⁸ such that each variant could be identified. Reads including variants were replaced with a corresponding reference read containing no identifiable SNPs. Post processing of the alignment was done by the SplitNCigarReads tool in GATK to split and trim intronic reads. Freebayes⁹ was run using the GRCh38 as the reference genome to identify the sites of variants. These sites were then identified in the STAR aligned BAM file and a vcf file created from variants being called from the BAM file. A custom script took the vcf file and identified each point of variation in the BAM file. If the position had an alternative allele it was flipped to the reference nucleotide. If there is no variant in the position or the reference allele is present the read was written as is. This output a new BAM file which was converted to a newly scrubbed fastq file.

### Bulk RNA-Seq PCA and Differential Gene Expression

Differentially expressed genes were identified using the DEseq2 R package¹⁰, after correcting for the effect of different patient donors. P-values were corrected for multiple hypotheses testing using the Benjamini & Hochberg (1995) method. Genes with an FDR (q-value) less than 0.05 were considered significant. PCA plots were constructed by DESeq using linear model batch corrected data from LIMMA R package¹¹. The contribution of each covariate on the principle components was calculated using the SWAMP R package¹². Row normalized (Complete linkage, average linkage, ward method), heat maps were constructed using the gene expression (TPM) data generated by RSEM. Genes were classified using their Gene ontology annotation^{13,14} and the gene cards database (www.genecards.org).

### Gene Set Enrichment Analysis

Gene set enrichment analysis (GSEA) was performed at each time point against a gene list of early polarizing T_{H}1 genes¹⁵ with the Deseq2 generated DE gene lists ranked by log₂(fold change). Analysis was performed using the desktop GSEA (v3.0). To identify MSigDB¹⁶ gene sets enriched in significantly differentially expressed genes (FDR<0.05) between any two CARs we ran the GSEA software¹⁷.

### Single Cell RNA-Seq

CAR T cell were sorted into PBS with 0.04% BSA and live cells were processed directly for droplet-based 3′ end parallel scRNA-seq. We used 10× genomics Chromium Single Cell 3′ Library & Gel bead Kit V2 according to manufacturer's protocol. An input of 10,000 cells was added to each 10× channel with a median recovery of 4,051 cells. Libraries were sequenced on an Illumaina HiSeq (132 bp reads) at the Broad (Gienomics Platform.

### ScRNA-Seq Initial Data Processing and QC

Gene counts were obtained by aligning reads to the human genome GRCh38 using CellRanger software (v2.2) (10× Genomics, Single cell 3′ v2)¹⁸. The scCloud pipeline¹⁹ was used for an overview analysis and to create a Seurat_h5ad file that could be loaded into R as a Seurat object for further QC and analysis. Seurat pipeline version 2.3.4²⁰ was used to remove poor quality cells (200<nUMI<6000 and ribosomal RNA<10% of the reads). Reads were normalized using Seurat's normalization function based on Log normalization with a scale factor of 10,000. For each analysis (all samples, resting samples or CAR stimulated samples) samples were selected and batch corrected for donor variation. Seurat's FindVariableGenes( ) function was used to determine the variable genes. Data was scaled using the ScaleData( ) function in Seurat using the identified variable genes.

### Batch Correction, Dimensionality Reduction, Clustering, and Visualization

The union of the top 1,000 most variable genes from the samples in each donor was used to run Canonical Correlation Analysis (CCA) using the RunCCA( ) function in Seurat. The donors' CCA subspaces were aligned using cca.aligned dimensional reduction function in Seurat which was then be used to run tSNE and for clustering purposes with the following parameter settings: reduction.use=“cca.aligned”, dims.use=1:20, do.fast=T. Clustering was performed using Seurat function FindClusters( ) with parameters: reduction.type=“cca.aligned”, resolution=0.6, dims.use=1:20.

### Topic Modeling

A Latent Dirichlet Allocation (LDA) topic model was fit on the sparse matrix from the CAR stimulated samples that included all genes as described²¹. Topics for set K number of topics ranging from 4 to 20 with the tolerance parameter (Tol) set to 0.1 was calculated. K was chosen to be 16 by calculating the Akaike and Bayesian information criteria for each K tested and K was set as the value where the AIC curve changed from a steep gradient to a more gradual one.

### Network Analysis

Transcription factor(TF) network analysis was performed using Regnet database²² and an enrichment of TFs regulating the top 100 genes was looked for in each topic.

### Cytokine Detection of Stimulated T Cells

For cytokine release assays, T cells were stimulated in a 96 well plates with 100 000 T effector cells/well combined with irradiated target cells at a CAR T cell-to-target ratio of 1:1 for Nalm6 targets and 2:1 for U87 targets. Supernatants were harvested after 24 hours and frozen at −80C. Supernatants were analyzed for cytokine levels using FLEXMAP 3D® platform from Lumina Instrumentation (Thermo Fisher Scientific) according to manufacturer's instructions with a panel of the following cytokines: IL-1β, IL-2, IL-4, IL-5, IL-6, IL12p70, IL-13, IL-18, IFN-γ, GM-CSF, TNF-α, IL-10 and IL-21. Plates were read using xPONENT Software 4.1. All samples were in measured in technical triplicates and with N=3 normal donors. Triplicates measured were averaged before graphing with Prism (Graphpad software).

### Digital Droplet PCR

EGFR CAR T cells were transduced, expanded and rested for 7 days. 5e⁵ cells were collected by FACs and resuspended in 350 μl RLT buffer with 1% 2-mercaptoethanol. RNA was extracted and purified using RNAeasy kit (Qiagen) and cDNA was generated from 270ng of RNA/20 μl reaction using iScript Reverse Transcription supermix (Bio-Rad). Digital Droplet PCR was performed using ddPCR supermix with no dUTPs (Bio-Rad) with a QX200 Droplet Digital PCR (ddPCR™) System (Bio-Rad) platform for quantification. Droplet generation, PCR and detection of positive droplets were performed according to manufacturer's instructions (Instruction Manual, QX200™ Droplet Generator-Bio-Rad).

The cycling protocol was according the manufacturer's instructions with a 57° C. melting temperature. Human TBP was used at as the reference gene in each reaction, (HEX fluorophore: TBP PrimePCR™ ddPCR™ Expression Probe Assay: Unique Assay ID: dHsaCPE5058363 (Bio-Rad)). The following FAM fluorophore primer probes were used (IDT PrimeTime Std® qPCR Assay). Primers and probes are shown in Table 11.

TABLE 11
Primers and Probes

| Gene | Primer/Probe |
|---|---|
| CTLA-4 | PrimeTime Primer 1: |
| | CGG ACC TCA GTG GCT TTG |
| | (SEQ ID NO: 5) |
| | Prime Time Primer 2: |
| | TTC ATC CCT GTC TTC TGC |
| | AA (SEQ ID NO: 6) |
| | Prime Time Probe: |
| | /56-FAM/CG CCA GCT T/ |
| | Zen/T GTG TGT GAG TAT |
| | GC/3IABkFQ |
| | (SEQ ID NO: 7) |
| GZMB | PrimeTime Primer 1: |
| | CAG AGA CTT CTG ATC |
| | CCA GAT |
| | (SEQ ID NO: 8) |
| | Prime Time Primer 2: |
| | TCC TGA GAA GAT GCA |
| | ACC AAT |
| | (SEQ ID NO: 9) |
| | Prime Time Probe: |
| | /56-FAM/CC CGC CCC T/ |
| | Zen/A CAT GGC TTA TCT/ |
| | 3IABkFQ |
| | (SEQ ID NO: 10) |
| SOCS2 | PrimeTime Primer 1: |
| | GAT ATT GTT AGT AGG |
| | TAG TCT GAA TGC |
| | (SEQ ID NO: 11) |
| | PrimeTime Primer 2: |
| | GGA GCT CGG TCA GAC |
| | AG (SEQ ID NO: 12) |
| | PrimeTime Probe: |
| | /56-FAM/AA AGA GGC |
| | A/Zen/C CAG AAG GAA |
| | CTT TCT TGA /3IABkFQ/ |
| | (SEQ ID NO: 13) |
| SDC4 | PrimeTime Primer 1: |
| | GGT ACA TGA GCA GTA |
| | GGA TCA G |
| | (SEQ ID NO: 14) |
| | PrimeTime Primer 2: |
| | GCA GCA ACA TCT TTG |
| | AGA GAA C |
| | (SEQ ID NO:15) |
| | PrimeTime Probe: |
| | /56-FAM/CC ACG ATG |
| | C/Zen/C ACC CAC AAT |
| | CAG A/3IABkFQ/ |
| | (SEQ ID NO: 16) |
| GGT1 | PrimeTime Primer 1: |
| | TTC AGG TCC TCA GCT |
| | GTC A |
| | (SEQ ID NO: 17) |
| | PrimeTime Primer 2: |
| | TGG CTG ACA CCT ACG |
| | AGA C |
| | (SEQ ID NO: 18) |
| | PrimeTime Probe: |
| | /56-FAM/CC GCC TGG |
| | A/Zen/T GTC CTT CAC |
| | AAT CT/3IABkFQ/ |
| | (SEQ ID NO: 19) |
| TNFRSF10A | PrimeTime Primer 1: |
| | GTC CAT TGC CTG ATT |
| | CTT TGT G |
| | (SEQ ID NO: 20) |
| | PrimeTime Primer 2: |
| | GTC AGT GCA AAC CAG |
| | GAA CT |
| | (SEQ ID NO: 21) |
| | PrimeTime Probe: |
| | /56-FAM/AT TCT GCT |
| | G/Zen/A GAT GTG CCG |
| | GAA GT/3IABkFQ/ |
| | (SEQ ID NO: 22) |
| BIRC3 | PrimeTime Primer 1: |
| | GTA GAT GAG GGT AAC |
| | TGG CTT G |
| | (SEQ ID NO: 23) |
| | PrimeTime Primer 2: |
| | GGT GTT GGG AAT CTG |
| | GAG ATG |
| | (SEQ ID NO: 24) |
| | PrimeTime Probe: |
| | /56-FAM/CC TTG GAA |
| | A/Zen/C CAC TTG |
| | GCA TGT TGA/3IABkFQ/ |
| | (SEQ ID NO: 25) |

### Statistical Analysis

Data are presented as means±SEM as stated in the figure legends. Unless specifically indicated, comparison between different groups was conducted with two-tailed, paired Student's t-tests. Unless otherwise stated, P values below 0.05 were considered significant. If adjustments for multiple comparisons were needed, they were performed using Holm-Bonferroni method with adjusted-p (adj-p)<0.05 was considered significant. Statistical analysis was performed with Prism (Graphpad software) and Holm-Bonferroni adj-p and Chi Square distribution were calculated with R software package. Wilcoxon rank sum-test was used to calculate p-values for single cell gene expression data with R software package.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A method of identifying a candidate CAR T cell comprising:
measuring expression of a gene signature of a CAR T cell and identifying the CAR T cell as the candidate CAR T cell if the CAR T cell a gene signature selected from:
   g) a CD3ζ CAR T gene signature,
   h) a costimulatory molecule gene signature,
   i) a T_{H}1 response gene signature,
   j) a T_{H}2 response gene signature,
   k) a T cell activation gene signature,
   l) any combination thereof.
      2. The method of aspect 1, wherein the CD3ζ CAR T gene signature comprises one or more signature genes selected from the group consisting of:
      ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof.
      3. The method of any of the preceeding aspects, wherein one or more signature genes in the CD3ζ CAR T gene signature are up-regulated, down-regulated, or both.
      4. The method of any of the preceeding aspects, wherein the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof.
      5. The method of any of the preceeding aspects, wherein the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof.
      6. The method of any of the preceeding aspects, wherein the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof.
      7. The method of any of the preceeding aspects, wherein the CD3ζ CAR T gene signature comprises ZP3 or GGT1.
      8. The method of any of the preceeding aspects, wherein the CD3ζ CAR T gene signature comprises CCL3, CCL4, GZMB, XCL1, ZBED2, IFNG, or any combination thereof.
      9. The method of any of the preceeding aspects, wherein the costimulatory molecule gene signature comprises one or more signature genes of Table 7, Table 8, or any combination thereof.
      10. The method of any of the preceeding aspects, wherein one or more signature genes in the costimulatory molecule gene signature are up-regulated, down-regulated, or both.
      11. The method of any of the preceeding aspects, wherein the costimulatory molecule gene signature comprises a gene signature selected from the group consisting of:
      (a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6,ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
      (b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
      (c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, and any combination thereof;
      (d) JUN, GPA33, KRT1, EGR1, CIITA, UBD, KLHL23, SCD, HLA-DOA, ALPK, CXCL10, and any combination thereof;
      (e) JUN, EGR1, CIITA, GPA33, KRT1, and any combination thereof;
      (f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
      (g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
      (h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
      (i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
      (j) CXCL10, JUNB, NTRK2, MSC, VNN2 and any combination thereof;
      (k) JUNB, CXCL10, ENOX1, ENPP2, DDIT4, NTRK2, GCSAM, IL5, and any combination thereof;
      (l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
      (m) ENPP2, GJB2, C4orf26, MX1, NTRK2, JUNB, TNFRSF8, DGAT2, ELL2, IL4I1, ITPR1, HLA-DRB6, and any combination thereof; and
      (n) CIITA, CD74, HLA-DMB, HLA-DPB1, HLA-DQA2, HLA-DRB1, HLA-DRB5, HLA-DOA, HLA-DRA, HLA-DRB6, and any combination thereof.
      12. The method of any of the preceeding aspects, wherein the CAR T cell is CD4+.
      13. The method of any of the preceeding aspects, wherein the gene signature is any one of gene signatures (a)-(i).
      14. The method of any of the preceeding aspects, wherein the CAR T cell is CD8+.
      15. The method of any of the preceeding aspects, wherein the gene signature is any one of gene signatures (a), (b), (c), (j), (k), (l), or (m).
      16. The method of any of the preceeding aspects, wherein the CAR T cell is unstimulated.
      17. The method of any of the preceeding aspects, wherein the gene signature is any one of gene signatures (a), (d), (e), or (j).
      18. The method of any of the preceeding aspects, wherein the CAR T cell is stimulated.
      19. The method of any of the preceeding aspects, wherein the gene signature is any one of gene signatures (b), (c), (f), (g), (h), (i), (k), (l), (m).
      20. The method of any of the preceeding aspects, wherein the CAR T cell expresses a CD28ζ co-stimulatory molecule.
      21. The method any of the preceeding aspects, wherein one or more genes in any one of gene signatures (a)-(i) is up-regulated, down-regulated, or both as compared to a CAR T cell expressing a BBζ co-stimulatory molecule.
      22. The method of any of the preceeding aspects, wherein LGMN, PDCD1, GPA33, KRT1, VNN2, C17orf-PLSCR3, and any combination thereof is up-regulated in the CART cell as compared to a CAR T expressing a BBζ co-stimulatory molecule.
      23. The method of any of the preceeding aspects, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is down-regulated in the CART cell as compared to a CAR T expressing a BBζ co-stimulatory molecule.
      24. The method of any of the preceeding aspects, wherein the CAR T cell expresses a BBζ co-stimulatory molecule.
      25. The method of any of the preceeding aspects, wherein the one or more genes in any one of gene signatures (a)-(i) is up-regulated, down-regulated, or both as compared to a CAR T cell expressing a CD28ζ co-stimulatory molecule.
      26. The method of any of the preceeding aspects, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is up-regulated in the CAR T cell.
      27. The method of any of the preceeding aspects, wherein LGMN, PDCD1, GPA33, KRT1, VNN2, C17orf-PLSCR3, and any combination thereof is down-regulated in the CART cell.
      28. The method of any of the preceeding aspects, wherein the T_{H}1 response gene signature comprises one or more signature genes selected from the group consisting of:

ERG1, TBX21, RORC, IL12RB2, GLIL1, EPPN2, DMD, IFNG, and any combination thereof.

29. The method of any of the preceeding aspects, wherein the CAR T cell expresses a BBζ co-stimulatory molecule.
30. The method of any of the preceeding aspects, wherein the CAR T cell is CD4+.
31. The method of any of the preceeding aspects, wherein the T_{H}2 response gene signature comprises one or more signature genes selected from the group consisting of: IL4, IL5, IL2, and any combination thereof.
32. The method of any of the preceeding aspects, wherein the CAR T cell expresses a CD28ζ co-stimulatory molecule.
33. The method of any of the preceeding aspects wherein the CAR T cell is CD4+.
34. The method of any of the preceeding aspects, wherein the T cell activation gene signature comprises one or more genes selected from Table 3, Table 4, or a combination thereof.
35. The method of any of the preceeding aspects, wherein the T cell activation gene signature comprises one or more genes selected from the group consisting of:

IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof.

36. The method of any of the preceeding aspects, wherein the stimulated CAR T cell was generated by stimulating the CAR T cell through a T cell receptor of the CAR T cell.
37. The method of any of the preceeding aspects, wherein IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are upregulated as compared to a CAR T cell stimulated through a CAR of the CAR T cell.
38. The method of any of the preceeding aspects, wherein the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1 TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

39. The method of any of the preceeding aspects, wherein the CAR T is a stimulated CAR T cell, wherein the stimulated CAR T cell was generated by stimulating a chimeric antigen receptor of CAR T cell.
40. The method of any of the preceeding aspects, wherein measuring expression of a gene signature comprises bulk RNA sequencing, single cell RNA sequencing (scRNA-seq), or both.
41. The method of any of the preceeding aspects, further comprising isolating an identified candidate CAR T cell or a population thereof to obtain an isolated candidate CAR T cell or population thereof and optionally expanding the isolated candidate CAR T cell or population thereof to obtain an expanded candidate CAR T cell or population thereof.
43. The method of any of the preceeding aspects, further comprising administering the isolated candidate CAR T cell or population thereof or the expanded candidate CAR T cell or population thereof to a subject in need thereof.
44. The method of any of the preceeding aspects, wherein the subject in need thereof has a cancer.
45. A method of modulating a CAR T cell, comprising:
administering a modulating agent to a CAR T cell, wherein the modulating agent is capable of modifying the expression of one or more genes in the CAR T cell such that the CAR T cell comprises a gene signature selected from:
   a) a CD3ζ CAR T gene signature,
   b) a costimulatory molecule gene signature,
   c) a T_{H}1 response gene signature,
   d) a T_{H}2 response gene signature,
   e) a T cell activation gene signature, or
   f) any combination thereof.
      46. The method of aspect 45, wherein the CD3ζ CAR T gene signature comprises one or more signature genes selected from the group consisting of:
      ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, and any combination thereof.
      47. The method of any of aspects 45-46, wherein one or more signature genes in the CD3ζ CAR T gene signature are up-regulated, down-regulated, or both.
      48. The method of any of aspects 45-47, wherein the CD3ζ CAR T gene signature comprises one or more upregulated signature genes selected from the group consisting of: ASB2, BIRC3, CCL3, CCL4, GGT1, CTLA4, CSF2RB, GZMB, ZP3, SDC4, XCL1, ZBED2, IFNG, and any combination thereof.
      49. The method of any of aspects 45-48, wherein the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof.
      50. The method of any of aspects 45-49, wherein the CD3ζ CAR T gene signature comprises one or more downregulated signature genes selected from the group consisting of: CD248, FAM13A, LTB, OPN3, SOCS2, TNFRSF10A, PLXNA4, HPCAL1, and any combination thereof.
      51. The method of any of aspects 45-50, wherein the CD3ζ CAR T gene signature comprises ZP3 or GGT1.
      52. The method of any of aspects 45-51, wherein the CD3ζ CAR T gene signature comprises CCL3, CCL4, GZMB, XCL1, ZBED2, IFNG, or any combination thereof.
      53. The method of any of aspects 45-52, wherein the costimulatory molecule gene signature comprises one or more signature genes of Table 7, Table 8, or any combination thereof.
      54. The method of any of aspects 45-53, wherein one or more signature genes in the costimulatory molecule gene signature are up-regulated, down-regulated, or both.
      55. The method of any of aspects 45-54, wherein the costimulatory molecule gene signature comprises a gene signature selected from the group consisting of:
      (a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6,ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
      (b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
      (c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, and any combination thereof;
      (d) JUN, GPA33, KRT1, EGR1, CIITA, UBD, KLHL23, SCD, HLA-DOA, ALPK, CXCL10, and any combination thereof;
      (e) JUN, EGR1, CIITA, GPA33, KRT1, and any combination thereof;
      (f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
      (g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
      (h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
      (i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof.
      (j) CXCL10, JUNB, NTRK2, MSC, VNN2 and any combination thereof;
      (k) JUNB, CXCL10, ENOX1, ENPP2, DDIT4, NTRK2, GCSAM, IL5, and any combination thereof;
      (l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
      (m) ENPP2, GJB2, C4orf26, MX1, NTRK2, JUNB, TNFRSF8, DGAT2, ELL2, IL4I1, ITPR1, HLA-DRB6, and any combination thereof, and
      (n) CIITA, CD74, HLA-DMB, HLA-DPB1, HLA-DQA2, HLA-DRB1, HLA-DRB5, HLA-DOA, HLA-DRA, HLA-DRB6, and any combination thereof.
      56. The method of any of aspects 45-55, wherein the gene signature is any one of gene signatures (a)-(i).
      57. The method of any of aspects 45-56, wherein the gene signature is any one of gene signatures (a), (b), (c), (j), (k), (l), or (m).
      58. The method of any of aspects 45-57, wherein the gene signature is any one of gene signatures (a), (d), (e), or (j).
      59. The method of any of aspects 45-58, wherein the gene signature is any one of gene signatures (b), (c), (f), (g), (h), (i), (k), (l), or (m).
      60. The method of any of aspects 45-59, wherein one or more genes in any one of gene signatures (a)-(i) is overexperssed, underexpressed, or both as compared to an unmodified CAR T cell.
      61. The method of any of aspects 45-60, wherein LGMN, PDCD1, GPA33, KRT1, VNN2, C17orf-PLSCR3, and any combination thereof is overexpressed in the CART cell.
      62. The method of any of aspects 45-61, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is underexpressed in the CART cell.
      63. The method of any of aspects 45-62, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is overexpressed in the CAR T cell.
      64. The method of any of aspects 45-63, wherein LGMN, PDCD1, GPA33, KRT1, VNN2, C17orf-PLSCR3, and any combination thereof is underexpressed in the CART cell.
      65. The method of any of aspects 45-64, wherein the T_{H}1 response gene signature comprises one or more signature genes selected from the group consisting of:

ERG1, TBX21, RORC, IL12RB2, GLIL1, EPPN2, DMD, IFNG, and any combination thereof.

66. The method of any of aspects 45-65, wherein the T_{H}2 response gene signature comprises one or more signature genes selected from the group consisting of: IL4, IL5, IL2, and any combination thereof.
67. The method of any of aspects 45-66, wherein the T cell activation gene signature comprises one or more genes selected from Table 3, Table 4, or a combination thereof.
68. The method of any of aspects 45-67, wherein the T cell activation gene signature comprises one or more genes selected from the group consisting of:

IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 and an any combination thereof.

69. The method of any of aspects 45-68, wherein IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are overexpressed or underexpressed in the CAR T cell.
70. The method of any of aspects 45-69, wherein the T cell activation gene signature comprises one or more genes from a gene signature selected from the group consisting of:

(a) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1 TNFSF10, and combinations thereof;

(b) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, and any combination thereof, and

(c) both (a) and (b).

71. The method of any of aspects 45-70, wherein the modifying agent is a therapeutic antibody, antibody fragment, antibody-like protein scaffold, aptamer, polypeptide, protein, genetic modifying agent, small molecule, small molecule degrader, or combination thereof.
72. The method of any of aspects 45-71, wherein the genetic modifying agent is a CRISPR-Cas system, a TALEN, a Zn-finger nuclease, or a meganuclease.
73. An isolated or engineered CAR T cell obtained according the method of any of aspects 1-72.
74. A method of treating a disease in a subject in need thereof comprising:

administering an identified candidate cell obtained by the method as in any one of aspects 1-73 or an isolated or engineered CAR T cell as in aspect 73, or a cell population thereof to the subject.

75. The method of aspect 74, where the disease is a cancer.
76. The method of any of aspects 74-75, further comprising administering an additional agent, therapy, antineoplastic or antitumor agent or radiation and/or surgical therapy or an antigen or a neoantigen.
77. The method of any of aspects 74-76, wherein the additional agent, therapy, antineoplastic or antitumor agent or radiation and/or surgical therapy or an antigen or neoantigen is administered sequentially or concurrently.
78. The method of any of aspects 74-77, wherein the sequential administration comprises a time period of a day, two days, three days, four days, five days, six days, a week, two weeks, three weeks, four weeks, five weeks, six weeks, seven weeks, eight weeks, nine weeks, ten weeks, eleven weeks, twelve weeks, or more.
79. A method of screening for one or more agents capable of modifying a gene expression signature of a CAR T cell as in any one of aspects 45-72, comprising:

contacting an unmodified CAR T cell population with a test modulating agent or a library of modulating agents;

identifying candidate CAR T cells present in the CART T cell population by the method of any one of aspects 1-44; and

selecting modulating agents that result in increasing the number of candidate CAR T cells present in the CAR T cell population.

80. The method of any one of aspects 1-79, wherein the CAR T cell or population thereof is obtained from or derived from a subject to be treated.

1. A method of identifying a candidate CAR T cell comprising: measuring expression of a gene signature of a CAR T cell and identifying the CAR T cell as the candidate CAR T cell if the CAR T cell comprises a gene signature selected from:
m) a CD3ζ CAR T gene signature,
n) a costimulatory molecule gene signature,
o) a T_{H}1 response gene signature,
p) a T_{H}2 response gene signature,
q) a T cell activation gene signature, or
r) any combination thereof.

2. The method of claim 1, wherein the CD3ζ CAR T gene signature comprises
(a) one or more signature genes selected from the group consisting of: ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, or any combination thereof;
(b) one or more signature genes selected from the group consisting of: ASB2, BIRC3, CCL3, CCL4, GGT1, CTLA4, CSF2RB, GZMB, ZP3, SDC4, XCL1, ZBED2, IFNG, or any combination thereof;
(c) one or more signature genes selected from the group consisting of: CD248, FAM13A, LTB, OPN3, SOCS2, TNFRSF10A, PLXNA4, HPCAL1, or any combination thereof;
(d) ZP3 or GGT1; and
(e) CCL3, CCL4, GZMB, XCL1, ZBED2, IFNG, or any combination thereof.

3. The method of claim 2, wherein one or more signature genes
(a) in the CD3ζ CAR T gene signature are up-regulated, down-regulated, or both;
(b) in the costimulatory molecule gene signature are up-regulated, down-regulated, or both; or
(c) both (a) and (b).

4.-8. (canceled)

9. The method of claim 1, wherein the costimulatory molecule gene signature comprises
(a) one or more signature genes of Table 7, Table 8, or any combination thereof;
(b) one or more signature genes selected from the group consisting of: IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6,ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, and any combination thereof;
(c) one or more signature genes selected from the group consisting of: ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, and any combination thereof;
(d) one or more signature genes selected from the group consisting of: GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, and any combination thereof;
(e) one or more signature genes selected from the group consisting of: JUN, GPA33, KRT1, EGR1, CIITA, UBD, KLHL23, SCD, HLA-DOA, ALPK, CXCL10, and any combination thereof;
(f) one or more signature genes selected from the group consisting of: JUN, EGR1, CIITA, GPA33, KRT1, and any combination thereof;
(g) one or more signature genes selected from the group consisting of: C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, and any combination thereof;
(h) one or more signature genes selected from the group consisting of: ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, and any combination thereof;
(i) one or more signature genes selected from the group consisting of: GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, and any combination thereof;
(i) one or more signature genes selected from the group consisting of: GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, and any combination thereof;
(k) one or more signature genes selected from the group consisting of: CXCL10, JUNB, NTRK2, MSC, VNN2 and any combination thereof;
(l) one or more signature genes selected from the group consisting of: JUNB, CXCL10, ENOX1, ENPP2, DDIT4, NTRK2, GCSAM, IL5, and any combination thereof;
(m) one or more signature genes selected from the group consisting of: ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, and any combination thereof;
(n) one or more signature genes selected from the group consisting of: ENPP2, GJB2, C4orf26, MX1, NTRK2, JUNB, TNFRSF8, DGAT2, ELL2, IL4I1, ITPR1, HLA-DRB6, and any combination thereof; and
(o) one or more signature genes selected from the group consisting of: CIITA, CD74, HLA-DMB, HLA-DPB1, HLA-DQA2, HLA-DRB1, HLA-DRB5, HLA-DOA, HLA-DRA, HLA-DRB6, and any combination thereof.

10.-11. (canceled)

12. The method of claim 9, wherein the CAR T cell is CD4+ or is CD8+.

13. The method of claim 12, wherein the CAR T cell is CD4+ and the costimulatory molecule gene signature comprises (b), (c), (d), (e), (f), (g), (h), (i), or (j).

14. (canceled)

15. The method of claim 12, wherein the CAR T cell is CD8+ and the costimulatory molecule gene signature comprises (b), (c), (d), (k), (l), (m), or (n).

16. The method of claim 9, wherein the CAR T cell is unstimulated and optionally wherein the costimulatory molecule gene signature comprises (b), (e), (f), or (k).

17. (canceled)

18. The method of claim 9, wherein the CAR T cell is stimulated and optionally wherein the costimulatory molecule gene signature is any one of (c), (d), (g), (h), (i), (j), (l), (n) or (o).

19. (canceled)

20. The method of claim 9, wherein the CAR T cell expresses a CD28ζ co-stimulatory molecule, expresses a BBζ co-stimulatory molecule, or both.

21. The method of claim 20, wherein the CAR T cell expresses a CD28ζ co-stimulatory molecule and wherein one or more genes in any one of gene signatures (a)-(j) is up-regulated, down-regulated, or both as compared to a CAR T cell expressing a BBζ co-stimulatory molecule.

22. The method of claim 21, wherein LGMN, PDCD1, GPA33, KRT1, VNN2, C17orf-PLSCR3, and any combination thereof is up-regulated in the CART cell as compared to a CAR T expressing a BBζ co-stimulatory molecule or wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is down-regulated in the CART cell as compared to a CAR T expressing a BBζ co-stimulatory molecule.

23.-24. (canceled)

25. The method of claim 20, wherein the CAR T cell expresses a BBζ co-stimulatory molecule and wherein one or more genes in any one of gene signatures (a)-(i) is up-regulated, down-regulated, or both as compared to a CAR T cell expressing a CD28ζ co-stimulatory molecule.

26. The method of claim 25, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, and any combination thereof is up-regulated in the CAR T cell or wherein LGMN, PDCD1, GPA33, KRT1, VNN2, C17orf-PLSCR3, and any combination thereof is down-regulated in the CART cell.

27. (canceled)

28. The method of claim 1, wherein the T_{H}1 response gene signature comprises one or more signature genes selected from the group consisting of: ERG1, TBX21, RORC, IL12RB2, GLIL1, EPPN2, DMD, IFNG, and any combination thereof.

29. The method of claim 28, wherein the CAR T cell expresses a BBζ co-stimulatory molecule.

30. The method of claim 29, wherein the CAR T cell is CD4+.

31. The method of claim 1, wherein the T_{H}2 response gene signature comprises one or more signature genes selected from the group consisting of: IL4, IL5, IL2, and any combination thereof.

32. The method of claim 31, wherein the CAR T cell expresses a CD28ζ co-stimulatory molecule and is optionally CD4+.

33. (canceled)

34. The method of claim 1, wherein the T cell activation gene signature comprises one or more genes selected from
(a) Table 3, Table 4, or a combination thereof;
(b) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1, TNFSF10, or any combination thereof;
(c) IFNG, IL3, CCL4, XCL1, CSF2, XCL2, CCL3, LTA, GZMB, LAG3, TNFRSF9, PIM3, RGCC, NKG7, FABP5, NDFIP1, MIR155HG, SRGN, PSMA2, BCL2L1, or any combination thereof;
(d) both (b) and (c); or
(e) IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 or any combination thereof.

35.-39. (canceled)

40. The method of claim 1, wherein measuring expression of a gene signature comprises bulk RNA sequencing, single cell RNA sequencing (scRNA-seq), or both.

41. The method of claim 1, further comprising isolating an identified candidate CAR T cell or a population thereof to obtain an isolated candidate CAR T cell or population thereof optionally expanding the isolated candidate CAR T cell or population thereof to obtain an expanded candidate CAR T cell or population thereof, and optionally administering the isolated candidate CAR T cell or population thereof or the expanded candidate CAR T cell or population thereof to a subject in need thereof, wherein the subject in need thereof optionally has cancer.

42.-44. (canceled)

45. A method of modulating a CAR T cell, comprising: administering a modulating agent to a CAR T cell, wherein the modulating agent is capable of modifying the expression of one or more genes in the CAR T cell such that the CAR T cell comprises a gene signature selected from:
a) a CD3ζ CAR T gene signature,
b) a costimulatory molecule gene signature,
c) a T_{H}1 response gene signature,
d) a T_{H}2 response gene signature,
e) a T cell activation gene signature, or
f) any combination thereof.

46. The method of claim 45, wherein the CD3ζ CAR T gene signature comprises: one or more signature genes selected from:
(a) ASB2, BIRC3, CCL3, CCL4, GGT1, CTLA4, CSF2RB, GZMB, ZP3, SDC4, XCL1, ZBED2, IFNG, CD248, FAM13A, LTB, OPN3, SOCS2, TNFRSF10A, PLXNA4, HPCAL1, or any combination thereof;
(b) ASB2, BIRC3, CCL3, CCL4, GGT1, CTLA4, CSF2RB, GZMB, ZP3, SDC4, XCL1, ZBED2, IFNG, or any combination thereof;
(c) CD248, FAM13A, LTB, OPN3, SOCS2, TNFRSF10A, PLXNA4, HPCAL1, and or combination thereof;
(d) ZP3 or GGT1;
(e) CCL3, CCL4, GZMB, XCL1, ZBED2, IFNG, or any combination thereof; or
(f) one or more genes of Table 7, Table 8, or any combination thereof.

47. The method of claim 46, wherein one or more signature genes in the CD3ζ CAR T gene signature are up-regulated, down-regulated, or both.

48.-54. (canceled)

55. The method of claim 45, wherein the costimulatory molecule gene signature comprises a gene signature selected from:
(a) IL12RB2, JUN, EGR1, CORO7-PAM16, ARID5A, WNT5B, CDKN1A, JAKMIP1, ENPP2, JUNB, CHRNA6, C1orf56, FAIM3, FOS, MPZL1, VNN2, MPP7, EVI2A, DMD, CRMP1, IRF8, C4orf26, GCA, BATF3, EGR2, EGR3, SH3YL1, GIMAP2, NLN, RPS29, STMN3, LAIR1, ENOX1, ICAM1, ANKRD33B, PARP3, ITPRIPL1, ING4, ARHGAP10, ZNF672, PRDM1, RPL39, GJB2, FILIP1L, ATHL1, FOXP1, MAPKAPK5-AS1, BBS2, ALPK2, AMICA1, CDCP1, HBEGF, SULT1B1, LIF, CDK6, C16orf54, EVI2B, MINA, SLC16A3, LOC728875, CIITA, PIK3IP1, GNA15, CTTNBP2NL, HLA-DQA2, ABLIM1, RRN3P1, LINC00599, IL16, P2RY14, PRKCQ-AS1, ADCY1, GPA33, TNFSF10, FAM200B, TCEA3, TTC39C, TNFRSF8, MEGF6,ANKRD37, NTRK2, RALB, SNHG6, ANXA2R, PTBP1, MIR155HG, SOCS3, ZC4H2, SERINC5, SLC7A5, FASN, CYB5A, SDC, PLAGL2, or any combination thereof;
(b) ENPP2, ENOX1, DDIT4, JUNB, CIITA, DMD, GJB2, ARHGAP10, HLA-DQA2, GNA15, EGR1, JUN, LOC100129034, POU2F2, VOPP1, TPM4, E2F1, PLAUR, IL23R, CA2, BCL2A1, HLA-DPB1, HLA-DRB5, FILIP1L, DNAJC6, ATHL1, UBAC1, NR5A2, NTRK2, HLA-DRB6, LZTFL1, BTN2A2, UBE2F, ENPP1, ANKRD33B, LRRC32, HLA-DRA, LHFP, HLA-DRB1, ZNF704, TXLNG, ADA, GCSAM, C4orf26, CTH, ADRBK1, G0S2, HLA-DPA1, CD74, IL18RAP, ULBP2, F8, HLA-DOA, ARNTL2, RNF19B, IL4I1, TMEM178B, ODC1, NEK6, TBL1X, LINC00176, MED12L, DBNDD2, HBEGF, HLA-DQB2, TSHR, FSCN1, BACH2, MMD, CTTNBP2NL, RNF167, GPR132, AMICA1, ADAT2, GNPDA1, ZNF502, CXCR6, BCL2L11, PP7080, C10orf54, OSM, ANK3, EPDR1, MINA, PON2, FOXP1, ELL2, P2RY14, WWTR1, ANXA3, ENPP3, DDX4, USP18, ZDHHC9, BAG1, KIF1A, TBKBP1, KIAA1671, ADCY1, TMEM189, BA, MTSS1, or any combination thereof;
(c) GJB2, NTRK2, JUNB, DGAT2, AMICA1, MSC, SH3BP5, ELL2, DNAJC6, IL12RB2, OAS3, G0S2, HLA-DQA2, DMD, HLA-DRB6, FUOM, HLA-DRA, IL4I1, ENPP2, P2RY14, C4orf26, ADCY1, MPZL1, PDE4DIP, LAIR1, IL23R, NFE2L3, ADA, ITPR1, HLA-DRB5, TMEM165, HLA-DPA1, PDE4A, HLA-DPB1, HLA-DRB1, ZFAND5, MINA, RALB, PRKCDBP, TMEM178B, DGCR6L, ARHGEF10, ANK3, TNFRSF8, EHD4, ARID5A, IL21, SPECC1, CIITA, CTTNBP2NL, GCSAM, SH2D1A, JUN, BIRC3, EMC8, ARHGAP10, C15orf48, FBXO4, KLHDC2, HAGHL, UPP1, RNF19B, RNASE6, TNIP2, BIK, SCML4, USP48, P2RY11, MATN4, NCALD, NFKBIE, CCDC88A, LOC100132891, LHFP, MINOS1, COL6A5, HLA-DQB2, KCNA3, SLBP, MTSS1, PAX8, FAS, DDHD2, IL21R, PIK3C2B, C9orf16, HIVEP1, GPR132, WNT5B, NDFIP2, PLK3, NOD2, UBE2J1, PNKD, NCOA5, BATF3, VCAM1, EGR1, IRF4, EVC, RUNX2, IL31RA, ZNRF1, KDSR, IGFLR1, SEPW1, IFIH1, JMY, LOC100506668, ETV6, DENND4A, RGL4, GLUL, NOMO3, CD74, ZDHHC3, NOTCH2, MAF1, CXCL10, MLLT3, HMSD, ZNF704, INSIG1, TACO1, TRIM14, TARSL2, PON2, RPL37A, SLC25A10, RGMB, TTC39C, AKIRIN1, FAM173B, CLPTM1, ANXA11, FBXO32, GET4, RCN2, ALDH4A1, CD58, LYSMD2, NFKBIA, MKNK1, TMEM121, PROSER1, CIRBP, MTDH, PPP1CC, PIR, APOBR, B3GNT2, DECR1, MAP3K6, TAF4B, PCED1B, OGFOD3, C1orf228, DNAJC5B, SLC25A22, BCL2L11, RPL21P28, TMOD1, CDKN2A, LRP8, MLLT4, ADAP1, JAK1, IFI44, MROH8, or any combination thereof;
(d) JUN, GPA33, KRT1, EGR1, CIITA, UBD, KLHL23, SCD, HLA-DOA, ALPK, CXCL10, or any combination thereof,
(e) JUN, EGR1, CIITA, GPA33, KRT1, or any combination thereof;
(f) C17orf61-PLSCR3, ENPP2, FILIP1L, HLA-DQA2, UBD, CIITA, GJB2, P2RY14, IL4I1, HLA-DOA, ENOX1, HLA-DRA, NTRK2, HLA-DRB1, COL6A1, DMD, BTN2A2, HLA-DPB1, HLA-DMB, HLA-DRB5, HLA-DQB2, JUN, GCSAM, HLA-DPA1, DDIT4, HLA-DRB6, C7orf55-LUC7L2, BCL2A, KRT7, or any combination thereof;
(g) ENPP2, FIKIP1L, HLA-DQA2, UBD, CIITA, IL4I1, ENOX1, COL6A1, BTN2A2, HLA-DRB5, GJB2, P2RY14, HLA-DOA, HLA-DRA, NTRK2, HLA-DPB1, HLAP-DRB1, DMD, HLA-DMB, HLA-DQB2, C17orf61-PLSCR3, or any combination thereof;
(h) GJB2, UBD, NTRK, THY, HLA-DQA, HLA-DRA, G0S2, CXCL10, IER2, CIITA, DOHH, ADA, MSC, JUNB, DMD, CDK6, HLA-DRB1, HLA-DOA, SH3BP5, LGMN, ACSL1, ANXA3, HLA-DRB5, EMC8, FILIP1L, PDCD1, ANK3, HLA-DRB6, IFNG, MPZL1, TMEM165, NOD2, DGAT2, AKIRIN1, ELL2, MATN4, SREBF2, INSIG1, BATF3, HLA-DPB1, MAF1, HLA-DPA1, ADCY1, NFKBIA, JUN, P2RY14, ANXA11, COTL1, HMHA1, IL23R, GCSAM, ZFAND5, IL21, ACADVL, IL21R, SLBP, or any combination thereof;
(i) GJB2, UBD, NTRK2, THY1, HLA-DQA, G0S2, CXCL10, DOHH, MSC, DMD, HLA-DOA, ANXA3, FILIP1L, IFNG, NOD2, TMEM165, SH3BP5, HLA-DRB1, JUNB, CDK6, ACSL, HLA-DRB5, HLA-DRB6, ANK3, MPZ1, LGMN, PDCD1, or any combination thereof;
(j) CXCL10, JUNB, NTRK2, MSC, VNN2, or any combination thereof,
(k) JUNB, CXCL10, ENOX1, ENPP2, DDIT4, NTRK2, GCSAM, IL5, or any combination thereof;
(l) ENPP2, GJB2, C4orf26, MX1, NTRK2, JUNB, TNFRSF8, DGAT2, ELL2, IL4I1, ITPR1, HLA-DRB6, GCSAM, ADCY1, HLA-DQA2, HLA-DRA, ANK3, or any combination thereof;
(m) ENPP2, GJB2, C4orf26, MX1, NTRK2, JUNB, TNFRSF8, DGAT2, ELL2, IL4I1, ITPR1, HLA-DRB6, or any combination thereof, or
(n) CIITA, CD74, HLA-DMB, HLA-DPB1, HLA-DQA2, HLA-DRB1, HLA-DRB5, HLA-DOA, HLA-DRA, HLA-DRB6, or any combination thereof.

56. The method of claim 55, wherein the gene signature is any one of gene signatures (a)-(i), any one of gene signatures (a), (b), (c), (j), (k), (l), or (m), any one of gene signatures (a), (d), (e), or (j), or any one of gene signatures (b), (c), (f), (g), (h), (i), (k), (l), or (m).

57.-59. (canceled)

60. The method of claim 55, wherein one or more genes in any one of gene signatures is overexperssed, underexpressed, or both as compared to an unmodified CAR T cell.

61. The method of claim 60, wherein LGMN, PDCD1, GPA33, KRT1, VNN2, C17orf-PLSCR3, or any combination thereof is overexpressed in the CART cell, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, or any combination thereof is underexpressed in the CART cell, wherein IL21, IL21R, IL12RB2, IL23R, ENPP2, CIITA, CD74, HLA-DMB, HLA-DPB1, HLA-DQA2, HLA-DRB1, HLA-DRB5, HLA-DOA, HLA-DRA, HLA-DRB6, or any combination thereof is overexpressed in the CAR T cell, or wherein LGMN, PDCD1, GPA33, KRT1, VNN2, C17orf-PLSCR3, or any combination thereof is underexpressed in the CART cell.

62.-64. (canceled)

65. The method of claim 45, wherein the T_{H}1 response gene signature comprises one or more signature genes selected from the group consisting of:
ERG1, TBX21, RORC, IL12RB2, GLIL1, EPPN2, DMD, IFNG, and any combination thereof.

66. The method of claim 45, wherein the T_{H}2 response gene signature comprises one or more signature genes selected from the group consisting of: IL4, IL5, IL2, and any combination thereof.

67. The method of claim 45, wherein the T cell activation gene signature comprises
(a) one or more genes selected from Table 3, Table 4, or a combination thereof;
(b) IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5 or any combination thereof;
(c) IL2RA, TUBA1B, ENO1, HSPD1, HSP90AA1, HSP90AB1, BATF3, NCL, AC133644.2, HNRNPAB, RANBP1, TPI1, NME1, TXN, CALR, SRM, RAN, CCND2, HSPE1, TNFSF10, or any combination thereof; or
(d) both (b) and (c).

68. (canceled)

69. The method of claim 68, wherein IFNG, CCL4, CCL3, IL3, XCL1, CSF2, GZMB, FABP5, XCL2, LTA, LAG3, MIR155HG, TNFRSF4, TNFRSF9, PIM3, IL13, ZBED2, PGAM1, EIF5A, IL5, are overexpressed or underexpressed in the CAR T cell.

70. (canceled)

71. The method of claim 45, wherein the modifying agent is a therapeutic antibody, antibody fragment, antibody-like protein scaffold, aptamer, polypeptide, protein, genetic modifying agent, small molecule, small molecule degrader, or combination thereof.

72. The method of claim 71, wherein the genetic modifying agent is a CRISPR-Cas system, a TALEN, a Zn-finger nuclease, or a meganuclease.

73. An isolated or engineered CAR T cell obtained according to the method of claim 45.

74. A method of treating a disease in a subject in need thereof comprising:
administering an isolated or engineered CAR T cell or a cell population thereof to the subject, wherein the isolated or engineered CAR T cell comprises a gene signature selected from: a CD3ζ CAR T gene signature, a costimulatory molecule gene signature, a T_{H}1 response gene signature, a T_{H}2 response gene signature, a T cell activation gene signature, or any combination thereof.

75. The method of claim 74, where the disease is a cancer.

76. The method of claim 74, further comprising administering an additional agent, therapy, antineoplastic or antitumor agent or radiation and/or surgical therapy or an antigen or a neoantigen.

77.-78. (canceled)

79. A method of screening for one or more agents capable of modifying a gene expression signature of a CAR T cell, comprising:
contacting an unmodified CAR T cell population with a test modulating agent or a library of modulating agents;
identifying candidate CAR T cells present in the CART T cell population by the method of claim 1; and
selecting modulating agents that result in increasing the number of candidate CAR T cells present in the CAR T cell population.

80. (canceled)
